(19) United States
(12) Patent Application Publication
(10) Pub. No.: US 2026/0218149 A1
(43) Pub. Date: Jul. 30, 2026

(54) CLASS II, TYPE V CRISPR SYSTEMS

(71) Applicant: Metagenomi Therapeutics, Inc., Emeryville, CA (US)

(72) Inventors: Brian C. Thomas, Berkeley, CA (US); Lisa Alexander, Albany, CA (US); Alan Brooks, Clayton, CA (US); Christopher Brown, Albany, CA (US); Cristina Noel Butterfield, Oakland, CA (US); Cindy Castelle, San Francisco, CA (US); Audra Devoto, King City, CA (US); Daniela S.A. Goltsman, Oakland, CA (US); Paula B. Matheus Carnevali, Pinole, CA (US); Isabel Nocedal, Oakland, CA (US); Morayma Temoche-Diaz, Emeryville, CA (US)

(21) Appl. No.: 19/099,615

(22) PCT Filed: Jul. 28, 2023
(86) PCT No.: PCT/US2023/071286

§ 371 Date: Jan. 29, 2025

Related U.S. Application Data
(60) Provisional application No. 63/369,920, filed on Jul. 29, 2022.
(60) Provisional application No. 63/386,298, filed on Dec. 6, 2022.
(60) Provisional application No. 63/484,168, filed on Feb. 9, 2023.

(51) Int. Cl. C12N 9/22; C07K 14/705; C07K 14/76; C12N 5/0783; C12N 5/0789; C12N 9/04; C12N 15/113; C12N 15/88

(52) U.S. Cl. CPC C12N 9/226; C07K 14/70596; C07K 14/76; C12N 5/0636; C12N 5/0647; C12N 9/0006; C12N 15/1138; C12N 15/88; C12Y 101/03015; C12N 2310/20; C12N 2510/00

(57) ABSTRACT

Described herein are methods, compositions, and systems derived from uncultivated microorganisms useful for gene editing.

### CROSS-REFERENCE

This application is related to PCT Application Nos. PCT/US21/21259 and PCT/US22/31849; and U.S. Provisional Application No. 63/369,920, filed on Jul. 29, 2022; each of which is incorporated by reference herein in its entirety.

### BACKGROUND

Cas enzymes along with their associated Clustered Regularly Interspaced Short Palindromic Repeats (CRISPR) guide ribonucleic acids (RNAs) appear to be a pervasive (~45% of bacteria, ~84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SEQUENCE LISTING

The instant application contains a Sequence Listing which has been submitted electronically in XML format and is hereby incorporated by reference in its entirety. Said XML copy, created on Dec. 1, 2022, is named 55921-755.102_SL.xml and is 14,705,179 bytes in size.

### SUMMARY

In some aspects, the present disclosure provides for an engineered nuclease system comprising: (a) an endonuclease comprising a sequence having at least about 80%, at least about 81%, at least about 82%, at least about 83%, at least about 84%, at least about 85% at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 6274-6281 or 6340-6551, or a variant thereof, wherein said endonuclease is a class 2, type V endonuclease, or a nucleotide sequence encoding said endonuclease; and (b) an engineered guide RNA, wherein said engineered guide RNA is configured to form a complex with said endonuclease and said engineered guide RNA comprises a spacer sequence configured to hybridize to a target nucleic acid sequence. In some embodiments, said endonuclease comprises a sequence having at least about 80%, at least about 81%, at least about 82%, at least about 83%, at least about 84%, at least about 85% at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 6274-6281. In some embodiments, said endonuclease is configured to bind to a protospacer adjacent motif (PAM) sequence having at least about 80%, at least about 81%, at least about 82%, at least about 83%, at least about 84%, at least about 85% at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 6332-6339. In some aspects, the present disclosure provides for an engineered nuclease system comprising: (a) an endonuclease comprising a PI (PAM interacting) domain having at least about 80%, at least about 81%, at least about 82%, at least about 83%, at least about 84%, at least about 85% at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a PI domain of any one of SEQ ID NOs: 2811, 2819, 2878, 2916, 2963, 3009, 6274, 6275, 6276, 6279, 6280, or 6281, or a variant thereof, or a nucleotide sequence encoding said endonuclease, wherein said endonuclease is a class 2, type V endonuclease and said endonuclease is configured to be selective for a 5′ PAM of any one of SEQ ID NOs: 6326-6339; and (b) an engineered guide RNA, wherein said engineered guide RNA is configured to form a complex with said endonuclease and said engineered guide RNA comprises a spacer sequence configured to hybridize to a target nucleic acid sequence. In some embodiments, said endonuclease comprises a sequence having at least about 80%, at least about 81%, at least about 82%, at least about 83%, at least about 84%, at least about 85% at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 2811, 2819, 2878, 2916, 2963, 3009, 6274, 6275, 6276, 6279, 6280, or 6281, or a variant thereof. In some embodiments, said guide RNA comprises a sequence with at least about 80%, at least about 81%, at least about 82%, at least about 83%, at least about 84%, at least about 85% at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to non-degenerate nucleotides of any one of SEQ ID NOs: 6284-6325. In some embodiments, said guide RNA spacer sequence comprises a sequence complementary to a eukaryotic, fungal, plant, mammalian, or human genomic polynucleotide sequence. In some embodiments, said endonuclease comprises at least one of a S168R, E172R, N577R, or Y170R mutation when a sequence of said endonuclease is optimally aligned to SEQ ID NO: 215. In some embodiments, the engineered nuclease system further comprises a single- or double-stranded DNA repair template comprising from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence. In some embodiments, said first or second homology arm comprises a sequence of at least 40, 80, 120, 150, 200, 300, 500, or 1,000 nucleotides. In some embodiments, said first and second homology arms are homologous to a genomic sequence of a prokaryote, bacteria, fungus, or eukaryote.

In some aspects, the present disclosure provides for an engineered nuclease system comprising: (a) an endonuclease comprising a RuvC domain, wherein the endonuclease is derived from an uncultivated microorganism, and wherein the endonuclease is a Cas12a endonuclease; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the Cas12a endonuclease comprises the sequence GWxxxK. In some embodiments, the engineered guide RNA comprises UCUAC[N₃₋₅]GUAGAU (N₄). In some embodiments, the engineered guide RNA comprises CCUGC[N₄]GCAGG (N₃₋₄).

In some aspects, the present disclosure provides for an engineered nuclease system comprising: (a) an endonuclease having at least 75% sequence identity to any one of SEQ ID NOs: 1-3470 or a variant thereof; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the endonuclease comprises a RuvCI, II, or III domain. In some embodiments, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a RuvCI, II, or III domain of any one of SEQ ID NOs: 1-3470 or a variant thereof. In some embodiments, the RuvCI domain comprises a D catalytic residue. In some embodiments the RuvCII domain comprises an E catalytic residue. In some embodiments the RuvCIII domain comprises a D catalytic residue. In some embodiments, said RuvC domain does not have nuclease activity. In some embodiments, said endonuclease further comprises a WED II domain having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a WED II domain of any one of SEQ ID NOs: 1-3470 or a variant thereof.

In some aspects, the present disclosure provides for an engineered nuclease system comprising: (a) an endonuclease configured to bind to a protospacer adjacent motif (PAM) sequence comprising any one of SEQ ID NOs: 3862-3913, wherein the endonuclease is a class 2, type V Cas endonuclease; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the endonuclease further comprises a zinc finger-like domain. In some embodiments, the guide RNA comprises a sequence with at least 80% sequence identity to the non-degenerate nucleotides of any one of SEQ ID NOs: 3471, 3551-3559, 3608-3609, 3612, 3636-3637, 3640-3641, 3644-3645, 3648-3649, 3652-3653, 3656-3657, 3660-3661, 3664-3667, 3671-3672, -3678, 3695-3696, 3729-3730, 3734-3735, and 3851-3857.

In some aspects, the present disclosure provides for an engineered nuclease system comprising: (a) an engineered guide RNA comprising a sequence with at least 80% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, or 3851-3857, and (b) a class 2, type V Cas endonuclease configured to bind to the engineered guide RNA. In some embodiments, the endonuclease is configured to bind to a protospacer adjacent motif (PAM) sequence comprising any one of SEQ ID NOs: 3863-3913. In some embodiments, the guide RNA comprises a sequence complementary to a eukaryotic, fungal, plant, mammalian, or human genomic polynucleotide sequence. In some embodiments, the guide RNA is 30-250 nucleotides in length. In some embodiments, the endonuclease comprises one or more nuclear localization sequences (NLSs) proximal to an N- or C-terminus of the endonuclease. In some embodiments, the NLS comprises a sequence at least 80% identical to a sequence from the group consisting of SEQ ID NO: 3938-3953. In some embodiments, the endonuclease comprises at least one of the following mutations: S168R, E172R, N577R, or Y170R when a sequence of the endonuclease is optimally aligned to SEQ ID NO: 215. In some embodiments, the endonuclease comprises the mutations S168R and E172R when a sequence of the endonuclease is optimally aligned to SEQ ID NO: 215. In some embodiments, the endonuclease comprises the mutations N577R or Y170R when a sequence of the endonuclease is optimally aligned to SEQ ID NO: 215. In some embodiments, the endonuclease comprises the mutation S168R when a sequence of the endonuclease is optimally aligned to SEQ ID NO: 215. In some embodiments, the endonuclease does not comprise a mutation of E172, N577, or Y170. In some embodiments, the engineered nuclease system further comprises a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides. In some embodiments, the first and second homology arms are homologous to a genomic sequence of a prokaryote, bacteria, fungus, or eukaryote. In some embodiments, the single- or double-stranded DNA repair template comprises a transgene donor. In some embodiments, the engineered nuclease system further comprises a DNA repair template comprising a double-stranded DNA segment flanked by one or two single-stranded DNA segments. In some embodiments, single-stranded DNA segments are conjugated to the 5′ ends of the double-stranded DNA segment. In some embodiments, the single stranded DNA segments are conjugated to the 3′ ends of the double-stranded DNA segment. In some embodiments, the single-stranded DNA segments have a length from 4 to 10 nucleotide bases. In some embodiments, the single-stranded DNA segments have a nucleotide sequence complementary to a sequence within the spacer sequence. In some embodiments, the double-stranded DNA sequence comprises a barcode, an open reading frame, an enhancer, a promoter, a protein-coding sequence, a miRNA coding sequence, an RNA coding sequence, or a transgene. In some embodiments, the double-stranded DNA sequence is flanked by a nuclease cut site. In some embodiments, the nuclease cut site comprises a spacer and a PAM sequence. In some embodiments, the system further comprises a source of Mg²⁺. In some embodiments, the guide RNA comprises a hairpin comprising at least 8, at least 10, or at least 12 base-paired ribonucleotides. In some embodiments, the hairpin comprises 10 base-paired ribonucleotides. In some embodiments: (a) the endonuclease comprises a sequence at least 75%, 80%, or 90% identical to any one of SEQ ID NOs: 141, 215, 229, 261, or 1711-1721 or a variant thereof, and (b) the guide RNA structure comprises a sequence at least 80%, or 90% identical to the non-degenerate nucleotides of any one of SEQ ID NOs: 3608-3609, 3853, or 3851-3857. In some embodiments, the endonuclease is configured to bind to a PAM comprising any one of SEQ ID NOs: 3863-3913. In some embodiments, the endonuclease is configured to bind to a PAM comprising SEQ ID NO: 3871. In some embodiments, the sequence identity is determined by a BLASTP, CLUSTALW, MUSCLE, MAFFT algorithm, or a CLUSTALW algorithm with the Smith-Waterman homology search algorithm parameters. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for an engineered guide RNA comprising: (a) a DNA-targeting segment comprising a nucleotide sequence that is complementary to a target sequence in a target DNA molecule; and (b) a protein-binding segment comprising two complementary stretches of nucleotides that hybridize to form a double-stranded RNA (dsRNA) duplex, wherein the two complementary stretches of nucleotides are covalently linked to one another with intervening nucleotides, and wherein the engineered guide ribonucleic acid polynucleotide is capable of forming a complex with an endonuclease having at least 75% sequence identity to any one of SEQ ID NOs: 1-3470, and targeting the complex to the target sequence of the target DNA molecule. In some embodiments, the DNA-targeting segment is positioned 3′ of both of the two complementary stretches of nucleotides. In some embodiments, the protein binding segment comprises a sequence having at least 70%, at least 80%, or at least 90% identity to the non-degenerate nucleotides of SEQ ID NO: 3608-3609. In some embodiments, the double-stranded RNA (dsRNA) duplex comprises at least 5, at least 8, at least 10, or at least 12 ribonucleotides.

In some aspects, the present disclosure provides for a deoxyribonucleic acid polynucleotide encoding the engineered guide ribonucleic acid polynucleotide described herein.

In some aspects, the present disclosure provides for a nucleic acid comprising an engineered nucleic acid sequence optimized for expression in an organism, wherein the nucleic acid encodes a class 2, type V Cas endonuclease, and wherein the endonuclease is derived from an uncultivated microorganism, wherein the organism is not the uncultivated organism. In some embodiments, the endonuclease comprises a variant having at least 70% or at least 80% sequence identity to any one of SEQ ID NOs: 1-3470. In some embodiments, the endonuclease comprises a sequence encoding one or more nuclear localization sequences (NLSs) proximal to an N- or C-terminus of the endonuclease. In some embodiments, the NLS comprises a sequence selected from SEQ ID NOs: 3938-3953. In some embodiments, the NLS comprises SEQ ID NO: 3939. In some embodiments, the NLS is proximal to the N-terminus of the endonuclease. In some embodiments, the NLS comprises SEQ ID NO: 3938. In some embodiments, the NLS is proximal to the C-terminus of the endonuclease. In some embodiments, the organism is prokaryotic, bacterial, eukaryotic, fungal, plant, mammalian, rodent, or human.

In some aspects, the present disclosure provides for an engineered vector comprising a nucleic acid sequence encoding a class 2, type V Cas endonuclease or a Cas12a endonuclease, wherein the endonuclease is derived from an uncultivated microorganism.

In some aspects, the present disclosure provides for an engineered vector comprising a nucleic acid described herein.

In some aspects, the present disclosure provides for an engineered vector comprising a deoxyribonucleic acid polynucleotide described herein. In some embodiments, the vector is a plasmid, a minicircle, a CELiD, an adeno-associated virus (AAV) derived virion, a lentivirus, or an adenovirus.

In some aspects, the present disclosure provides for a cell comprising a vector described herein.

In some aspects, the present disclosure provides for a method of manufacturing an endonuclease, comprising cultivating any of the host cells described herein.

In some aspects, the present disclosure provides for a method for binding, cleaving, marking, or modifying a double-stranded deoxyribonucleic acid polynucleotide, comprising: (a) contacting the double-stranded deoxyribonuclcic acid polynucleotide with a class 2, type V Cas endonuclcasc in complex with an engineered guide RNA configured to bind to the endonuclease and the double-stranded deoxyribonucleic acid polynucleotide; (b) wherein the double-stranded deoxyribonucleic acid polynucleotide comprises a protospacer adjacent motif (PAM); and (c) wherein the PAM comprises a sequence comprising any one of SEQ ID NOs: 3863-3913. In some embodiments, the double-stranded deoxyribonucleic acid polynucleotide comprises a first strand comprising a sequence complementary to a sequence of the engineered guide RNA and a second strand comprising the PAM. In some embodiments, the PAM is directly adjacent to the 5′ end of the sequence complementary to the sequence of the engineered guide RNA. In some embodiments, the PAM comprises SEQ ID NO: 3871. In some embodiments, the class 2, type V Cas endonuclease is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide. In some embodiments, the method comprising delivering to the target nucleic acid locus the engineered nuclease system described herein, wherein the endonuclease is configured to form a complex with the engineered guide ribonucleic acid structure, and wherein the complex is configured such that upon binding of the complex to the target nucleic acid locus, the complex modifies the target nucleic acid locus. In some embodiments, modifying the target nucleic acid locus comprises binding, nicking, cleaving, or marking the target nucleic acid locus. In some embodiments, the target nucleic acid locus comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC). In some embodiments, delivering the engineered nuclease system to the target nucleic acid locus comprises delivering the nucleic acid described herein or the vector described herein. In some embodiments, delivering the engineered nuclease system to the target nucleic acid locus comprises delivering a nucleic acid comprising an open reading frame encoding the endonuclease. In some embodiments, the nucleic acid comprises a promoter to which the open reading frame encoding the endonuclease is operably linked. In some embodiments, delivering the engineered nuclease system to the target nucleic acid locus comprises delivering a capped mRNA containing the open reading frame encoding the endonuclease. In some embodiments, delivering the engineered nuclease system to the target nucleic acid locus comprises delivering a translated polypeptide. In some embodiments, delivering the engineered nuclease system to the target nucleic acid locus comprises delivering a deoxyribonucleic acid (DNA) encoding the engineered guide RNA operably linked to a ribonucleic acid (RNA) pol III promoter. In some embodiments, the endonuclease induces a single-stranded break or a double-stranded break at or proximal to the target locus. In some embodiments, the endonuclease induces a staggered single stranded break within or 3′ to the target locus.

In some aspects, the present disclosure provides for a method of editing a TRAC locus in a cell, comprising contacting to the cell (a) an RNA-guided endonuclease; and (b) an engineered guide RNA, wherein the engineered guide RNA is configured to form a complex with the endonuclease and the engineered guide RNA comprises a spacer sequence configured to hybridize to a region of the TRAC locus, wherein the engineered guide RNA comprises a targeting sequence having at least 85% identity at least 18 consecutive nucleotides of any one of SEQ ID NOs: 4316-4369. In some embodiments, the RNA-guided nuclease is a Cas endonuclease. In some embodiments, the Cas endonuclease is a class 2, type V Cas endonuclease. In some embodiments, the class 2, type V Cas endonuclease comprises a RuvC domain comprising a RuvCI subdomain, a RuvCII subdomain, and a RuvCIII subdomain. In some embodiments, the class 2, type V Cas endonuclease comprises an endonuclease having at least 75% sequence identity to any one of SEQ ID NOs: 1-3470 or a variant thereof. In some embodiments, the engineered guide RNA further comprises a sequence with at least 80% sequence identity to at least 19 of the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, and 3851-3857. In some embodiments, the endonuclease comprises a sequence at least 75%, 80%, or 90% identical to any one of SEQ ID NOs: 141, 215, 229, 261, or 1711-1721 or a variant thereof. In some embodiments, the guide RNA structure comprises a sequence at least 80%, or at least 90% identical to at least 19 of the non-degenerate nucleotides of any one of SEQ ID NOs: 3608-3609, 3853, or 3851-3857. In some embodiments, the method further comprises contacting to the cell or introducing to the cell a donor nucleic acid comprising a cargo sequence flanked on a 3′ or 5′ end by sequence having at least 80% identity to any one of SEQ ID NOs: 4424 or 4425. In some embodiments, the cell is a peripheral blood mononuclear cell (PBMC). In some embodiments, the cell is a T-cell or a precursor thereof or a hematopoietic stem cell (HSC). In some embodiments, the cargo sequence comprises a sequence encoding a T-cell receptor polypeptide, a CAR-T polypeptide, or a fragment or derivative thereof. In some embodiments, the engineered guide RNA comprises a sequence having at least 80% identity to any one of SEQ ID NOs: 4370-4423. In some embodiments, the engineered guide RNA comprises the nucleotide sequence of any one of SEQ ID NOs: 4370-4423 comprising the corresponding chemical modifications listed in SEQ ID NOs: 4370-4423. In some embodiments, the engineered guide RNA comprises a targeting sequence having at least 80% sequence identity to any one of SEQ ID NOs: 4334, 4350, or 4324. In some embodiments, the engineered guide RNA comprises a sequence having at least 80% sequence identity to any one of SEQ ID NOs: 4388, 4404, or 4378. In some embodiments, the engineered guide RNA comprises the nucleotide sequence of any one of SEQ ID NOs: 4378, 4388, or 4404.

In some aspects, the present disclosure provides for an engineered nuclease system comprising: (a) an RNA-guided endonuclease; and (b) an engineered guide RNA, wherein the engineered guide RNA is configured to form a complex with the endonuclease and the engineered guide RNA comprises a spacer sequence configured to hybridize to a target nucleic acid sequence, wherein the engineered guide RNA comprises at least one of the following modifications: (i) a 2′-O methyl or a 2′-fluoro base modification of at least one nucleotide within the first 4 bases of the 5′ end of the engineered guide RNA or the last 4 bases of a 3′ end of the engineered guide RNA; (ii) a thiophosphate (PS) linkage between at least 2 of the first five bases of a 5′ end of the engineered guide RNA, or a thiophosphate linkage between at least two of the last five bases of a 3′ end of the engineered guide RNA; (iii) a thiophosphate linkage within a 3′ stem or a 5′ stem of the engineered guide RNA; (iv) a 2′-O methyl or 2′base modification within a 3′ stem or a 5′ stem of the engineered guide RNA; (v) a 2′-fluoro base modification of at least 7 bases of a spacer region of the engineered guide RNA; and (vi) a thiophosphate linkage within a loop region of the engineered guide RNA. In some embodiments, the engineered guide RNA comprises a 2′-O methyl or a 2′-fluoro base modification of at least one nucleotide within the first 5 bases of a 5′ end of the engineered guide RNA or the last 5 bases of a 3′ end of the engineered guide RNA. In some embodiments, the engineered guide RNA comprises a 2′-O methyl or a 2′-fluoro base modification at a 5′ end of the engineered guide RNA or a 3′ end of the engineered guide RNA. In some embodiments, the engineered guide RNA comprises a thiophosphate (PS) linkage between at least 2 of the first five bases of a 5′ end of the engineered guide RNA, or a thiophosphate linkage between at least two of the last five bases of a 3′ end of the engineered guide RNA. In some embodiments, the engineered guide RNA comprises a thiophosphate linkage within a 3′ stem or a 5′ stem of the engineered guide RNA. In some embodiments, the engineered guide RNA comprises a 2′-O methyl base modification within a 3′ stem or a 5′ stem of the engineered guide RNA. In some embodiments, the engineered guide RNA comprises a 2′-fluoro base modification of at least 7 bases of a spacer region of the engineered guide RNA. In some embodiments, the engineered guide RNA comprises a thiophosphate linkage within a loop region of the engineered guide RNA. In some embodiments, the engineered guide RNA comprises at least three 2′-O methyl or 2′-fluoro bases at the 5′ end of the engineered guide RNA, two thiophosphate linkages between the first 3 bases of the 5′ end of the engineered guide RNA, at least 4 2′-O methyl or 2′-fluoro bases at the 4′ end of the engineered guide RNA, and three thiophosphate linkages between the last three bases of the 3′ end of the engineered guide RNA. In some embodiments, the engineered guide RNA comprises at least two 2′-O-methyl bases and at least two thiophosphate linkages at a 5′ end of the engineered guide RNA and at least one 2′-O-methyl bases and at least one thiophosphate linkage at a 3′ end of the engineered guide RNA. In some embodiments, the engineered guide RNA comprises at least one 2′-O-methyl base in both the 3′ stem or the 5′ stem region of the engineered guide RNA. In some embodiments, the engineered guide RNA comprises at least one to at least fourteen 2′-fluoro bases in the spacer region excluding a seed region of the engineered guide RNA. In some embodiments, the engineered guide RNA comprises at least one 2′-O-methyl base in the 5′ stem region of the engineered guide RNA and at least one to at least fourteen 2′-fluoro bases in the spacer region excluding a seed region of the guide RNA. In some embodiments, the guide RNA comprises a spacer sequence targeting a VEGF-A gene. In some embodiments, the guide RNA comprises a spacer sequence having at least 80% identity to SEQ ID NO: 3985. In some embodiments, the guide RNA comprises the nucleotides of any one of SEQ ID NOs: 3985-3991 comprising the chemical modifications listed in SEQ ID NOs: 3985-3991. In some embodiments, the RNA-guided nuclease is a Cas endonuclease. In some embodiments, the Cas endonuclease is a class 2, type V Cas endonuclease In some embodiments, the class 2, type V Cas endonuclease comprises a RuvC domain comprising a RuvCI subdomain, a RuvCII subdomain, and a RuvCIII subdomain. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1721 or a variant thereof. In some embodiments, the engineered guide RNA comprises a sequence with at least 80% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, and 3851-3857. In some embodiments, the engineered guide RNA comprises a sequence with at least 80% sequence identity to the non-degenerate nucleotides of any one of SEQ ID NOs: 3608-3609, 3853, or 3851-3857.

In some aspects, the present disclosure provides for a host cell comprising an open reading frame encoding a heterologous endonuclease having at least 75% sequence identity to any one of SEQ ID NOs: 1-3470 or a variant thereof. In some embodiments, the endonuclease has at least 75% sequence identity to any one of SEQ ID NOs: 141, 215, 229, 261, or 1711-1721, or a variant thereof. In some embodiments, the host cell is an E. coli cell or a mammalian cell. In some embodiments, the host cell is an E. coli cell, wherein the E. coli cell is a ADE3 lysogen or the E. coli cell is a BL21 (DE3) strain. In some embodiments, the E. coli cell has an ompT lon genotype. In some embodiments, the open reading frame is operably linked to a T7 promoter sequence, a T7-lac promoter sequence, a lac promoter sequence, a tac promoter sequence, a tre promoter sequence, a ParaBAD promoter sequence, a PrhaBAD promoter sequence, a T5 promoter sequence, a cspA promoter sequence, an araP_{BAD} promoter, a strong leftward promoter from phage lambda (pL promoter), or any combination thereof. In some embodiments, the open reading frame comprises a sequence encoding an affinity tag linked in-frame to a sequence encoding the endonuclease. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the endonuclease via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell.

In some aspects, the present disclosure provides for a culture comprising any of the host cells described herein in compatible liquid medium.

In some aspects, the present disclosure provides for a method of producing an endonuclease, comprising cultivating any of the host cells described herein in compatible growth medium. In some embodiments, the method further comprises inducing expression of the endonuclease. In some embodiments, the inducing expression of the nuclease is by addition of an additional chemical agent or an increased amount of a nutrient, or by temperature increase or decrease. In some embodiments, an additional chemical agent or an increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises isolating the endonuclease. In some embodiments, the isolating comprises subjecting the protein extract to IMAC, ion-exchange chromatography, anion exchange chromatography, or cation exchange chromatography. In some embodiments, the open reading frame comprises a sequence encoding an affinity tag linked in-frame to a sequence encoding the endonuclease. In some embodiments, the affinity tag is linked in-frame to the sequence encoding the endonuclease via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the affinity tag by contacting a protease corresponding to the protease cleavage site to the endonuclease. In some embodiments, the affinity tag is an IMAC affinity tag. In some embodiments, the method further comprises performing subtractive IMAC affinity chromatography to remove the affinity tag from a composition comprising the endonuclease.

In some aspects, the present disclosure provides for a system comprising (a) a class 2, Type V-A Cas endonuclease configured to bind a 3- or 4-nucleotide PAM sequence, wherein the endonuclease has increased cleavage activity relative to sMbCas12a; and (b) an engineered guide RNA, wherein the engineered guide RNA is configured to form a complex with the class 2, Type V-A Cas endonuclease and the engineered guide RNA comprises a spacer sequence configured to hybridize to a target nucleic acid comprising a target nucleic acid sequence. In some embodiments, the cleavage activity is measured in vitro by introducing the endonucleases alongside compatible guide RNAs to cells comprising the target nucleic acid and detecting cleavage of the target nucleic acid sequence in the cells. In some embodiments, the class 2, Type V-A Cas endonuclease comprises a sequence having at least 75% identity to any one of 215-225 or a variant thereof. In some embodiments, the engineered guide RNA comprises a sequence having at least 80% identity to the non-degenerate nucleotides of SEQ ID NO: 3609. In some embodiments, the target nucleic acid further comprises a YYN PAM sequence proximal to the target nucleic acid sequence. In some embodiments, the class 2, Type V-A Cas endonuclease has at least about 5%, 10%, 20%, 30%, 40%, 50%, 60%, 70%, 80%, 90%, 100%, or 200%, or more increased activity relative to sMbCas12a.

In some aspects, the present disclosure provides for a system comprising: (a) a class 2, Type V-A′ Cas endonuclease; and (b) an engineered guide RNA, wherein the engineered guide RNA comprises a sequence having at least 80% identity to about 19 to about 25 or about 19 to about 31 consecutive nucleotides of a natural effector repeat sequence of a class 2, Type V Cas endonuclease. In some embodiments, the natural effector repeat sequence is any one of SEQ ID NOs: 3560-3572. In some embodiments, the class 2, Type V-A′ Cas endonuclease has at least 75% identity to SEQ ID NO: 126.

In some aspects, the present disclosure provides for a system comprising: (a) a class 2, Type V-L endonuclease, and (b) an engineered guide RNA, wherein the engineered guide RNA comprises a sequence having at least 80% identity to about 19 to about 25 or about 19 to about 31 consecutive nucleotides of a natural effector repeat sequence of a class 2, Type V Cas endonuclease. In some embodiments, the class 2, Type V-L endonuclease has at least 75% sequence identity to any one of SEQ ID NOs: 793-1163.

In some aspects, the present disclosure provides for a method of disrupting the VEGF-A locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the VEGF-A locus, wherein the engineered guide RNA comprises a targeting sequence having at least 80% identity to SEQ ID NO: 3985; or wherein the engineered guide RNA comprises the nucleotide sequence of any one of SEQ ID NOs: 3985-3991 comprising the chemical modifications listed in SEQ ID NOs: 3985-3991. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1721 or a variant thereof. In some embodiments, the engineered guide RNA comprises a sequence with at least 80% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, and 3851-3857. In some embodiments, the engineered guide RNA comprises a sequence with at least 80% sequence identity to the non-degenerate nucleotides of any one of SEQ ID NOs: 3608-3609, 3853, or 3851-3857.

In some aspects, the present disclosure provides for a method of disrupting a locus in a cell, comprising contacting to the cell a composition comprising: (a) a class 2, type V Cas endonuclease having at least 75% identity to any one of SEQ ID NOs: 215-225 or a variant thereof; and (b) an engineered guide RNA, wherein the engineered guide RNA is configured to form a complex with the endonuclease and the engineered guide RNA comprises a spacer sequence configured to hybridize to a region of the locus, wherein the class 2, type V Cas endonuclease has at least equivalent cleavage activity to spCas9 in the cell. In some embodiments, the cleavage activity is measured in vitro by introducing the endonucleases alongside compatible guide RNAs to cells comprising the target nucleic acid and detecting cleavage of the target nucleic acid sequence in the cells. In some embodiments, the composition comprises 20 pmoles or less of the class 2, type V Cas endonuclease. In some embodiments, the composition comprises 1 μmol or less of the class 2, type V Cas endonuclease.

In some aspects, the present disclosure provides for a method of disrupting a CD38 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the CD38 locus, wherein the engineered guide RNA is configured to hybridize to a sequence having at least 20-22 consecutive nucleotides having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4466-4503 and 5686; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4428-4465 and 5685. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4466, 4467, 4468, 4479, 4484, 4490, 4492, 4493, 4495, 4498. In some embodiments, the engineered guide RNA comprises a nucleotide sequence having at least 80% identity to any one of SEQ ID NOs: 4428, 4429, 4430, 4436, 4441, 4446, 4452, 4454, 4455, 4460, or 4461. In some embodiments, the cell is a eukaryotic cell, T-cell, hematopoietic stem cell, or precursor thereof.

In some aspects, the present disclosure provides for a method of disrupting a TIGIT locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the TIGIT locus, wherein the engineered guide RNA is configured to hybridize to a sequence having at least 20-22 consecutive nucleotides having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4521-4537; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4504-4520. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4521, 4527, 4528, 4535, or 4536. In some embodiments, the engineered guide RNA comprises a nucleotide sequence having at least 80% identity to any one of SEQ ID NOs: 4504, 4510, 4511, 4518, or 4519. In some embodiments, the cell is a eukaryotic cell, T-cell, hematopoietic stem cell, or precursor thereof.

In some aspects, the present disclosure provides for a method of disrupting an AAVS1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the AAVS1 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4569-4599; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4538-4568. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4574, 4577, 4578, 4579, 4582, 4584, 4585, 4586, 4587, 4589, 4590, 4591, 4592, 4593, 4595, 4596, or 4598. In some embodiments, the engineered guide RNA comprises a nucleotide sequence having at least 80% identity to any one of SEQ ID NOs: 4543, 4546, 4547, 4548, 4551, 4553, 4554, 4555, 4556, 4558, 4559, 4560, 4561, 4562, 4565, or 4567. In some embodiments, the cell is a eukaryotic cell, T-cell, hematopoietic stem cell, hepatocyte, or precursor thereof.

In some aspects, the present disclosure provides for a method of disrupting a B2M locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the B2M locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4676-4751; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4600-4675. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857 and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4676, 4678-4687, 4690, 4692, 4698-4707, 4720-4723, 4725-4726, 4732-4733, 4736-4737, 4741, or 4750-4751. In some embodiments, the engineered guide RNA comprises a nucleotide sequence having at least 80% identity to any one of SEQ ID NOs: 4600, 4602-4611, 4614, 4616, 4622-4631, 4644-4647, 4649-4650, 4656-4657, 4660-4661, 4665, or 4674-4675. In some embodiments, the cell is a eukaryotic cell, T-cell, hematopoietic stem cell, or precursor thereof.

In some aspects, the present disclosure provides for a method of disrupting a CD2 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the CD2 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4837-4921; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4752-4836. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4837, 4844, 4845, 4848, 4857-4858, 4883, 4887, 4892-4893, 4904-4909, 4914, 4916, or 4918. In some embodiments, the engineered guide RNA has at least 80% sequence identity to any one of SEQ ID NOs: 4752-4836 that target any one of SEQ ID NOs: 4837, 4844, 4845, 4848, 4857-4858, 4883, 4887, 4892-4893, 4904-4909, 4914, 4916, or 4918. In some embodiments, the cell is a eukaryotic cell, T-cell, hematopoietic stem cell, or precursor thereof.

In some aspects, the present disclosure provides for a method of disrupting a CD5 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the CD5 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4946-4969; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4922-4945. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 4946-4947, 4949, 4951, 4957-4960, 4963, 4967, or 4969. In some embodiments, the engineered guide RNA has at least 80% sequence identity to any one of SEQ ID NOs: 4922-4945 that target any one of SEQ ID NOs: 4946-4947, 4949, 4951, 4957-4960, 4963, 4967, or 4969. In some embodiments, the cell is a eukaryotic cell, T-cell, hematopoietic stem cell, or precursor thereof.

In some aspects, the present disclosure provides for a method of disrupting a mouse TRAC locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the mouse TRAC locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5126-5195, 5682, or 5684; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5056-5125, 5681, or 5683. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5126-5130, 5133-5143, 5147-5150, 5172-5173, 5184-5189, or 5192-5194. In some embodiments, the engineered guide RNA has at least 80% sequence identity to any one of SEQ ID NOs: 5056-5125 that target any one of SEQ ID NOs: 5126-5130, 5133-5143, 5147-5150, 5172-5173, 5184-5189, or 5192-5194. In some embodiments, the cell is a eukaryotic cell, T-cell, hematopoietic stem cell, or precursor thereof.

In some aspects, the present disclosure provides for a method of disrupting a mouse TRBC1 or TRBC2 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the mouse TRBC1 or TRBC2 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5211-5225 or 5247-5267; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5196-5210 or 5226-5246. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5211, 5213-5215, 5217, 5221, 5223, 5247, 5249-5250, 5252-5253, 5258-5259, or 5264. In some embodiments, the engineered guide RNA has at least 80% sequence identity to any one of SEQ ID NOs: 5196-5210 or 5226-5246 that target any one of SEQ ID NOs: 5211, 5213-5215, 5217, 5221, 5223, 5247, 5249-5250, 5252-5253, 5258-5259, or 5264. In some embodiments, the cell is a eukaryotic cell, T-cell, hematopoietic stem cell, or precursor thereof.

In some aspects, the present disclosure provides for a method of disrupting a human TRBC1 or TRBC2 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the human TRBC1 or TRBC2 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at last about 99% sequence identity to any one of SEQ ID NOs: 5661-5679; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5642-5660. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5661-5663, 5672-5675, or 5678. In some embodiments, the engineered guide RNA has at least 80% sequence identity to any one of SEQ ID NOs: 5642-5660 that target any one of SEQ ID NOs: 5661-5663, 5672-5675, or 5678. In some embodiments, the cell is a eukaryotic cell, T-cell, hematopoietic stem cell, or precursor thereof.

In some aspects, the present disclosure provides for a method of disrupting an HPRT locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the HPRT locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5562-5641; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5482-5561. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5562-5564 or 5568. In some embodiments, the engineered guide RNA has at least 80% sequence identity to any one of SEQ ID NOs: 5482-5561 that target any one of SEQ ID NOs: 5562-5564 or 5568. In some embodiments, the cell is a eukaryotic cell, hepatocyte, T-cell, hematopoietic stem cell, or precursor thereof.

In some aspects, the present disclosure provides for a method of disrupting an APO-A1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the APO-A1 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5861-5874; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5847-5860. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5861-5866 or 5868-5869. In some embodiments, the engineered guide RNA has at least 80% sequence identity to any one of SEQ ID NOs: 5847-5860 that target any one of SEQ ID NOs: 5861-5866 or 5868-5869. In some embodiments, the cell is a eukaryotic cell, hepatocyte, T-cell, hematopoietic stem cell, or precursor thereof.

In some aspects, the present disclosure provides for a method of disrupting an ANGPTL3 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the ANGPTL3 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5953-6030; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5875-5952. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the engineered guide RNA is configured to hybridize to a sequence having at least 20-22 consecutive nucleotides having at least 80% identity to any one of SEQ ID NOs: 5955-5963, 5968-5975, 5979-5987, 5989-5993, 5997, 5999, 6003-6010, 6014-6016, 6024-6025, or 6027-6030. In some embodiments, the engineered guide RNA has at least 80% sequence identity to any one of SEQ ID NOs: 5875-5952 that target any one of SEQ ID NOs: 5955-5963, 5968-5975, 5979-5987, 5989-5993, 5997, 5999, 6003-6010, 6014-6016, 6024-6025, or 6027-6030. In some embodiments, the cell is a eukaryotic cell, hepatocyte, T-cell, hematopoietic stem cell, or precursor thereof.

In some aspects, the present disclosure provides for a method of disrupting a human Rosa26 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the human Rosa26 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5013-5055; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4970-5012. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

In some aspects, the present disclosure provides for a method of disrupting a FAS locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the FAS locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5367-5465; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5268-5366. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

In some aspects, the present disclosure provides for a method of disrupting a PD-1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the PD-1 locus, wherein the engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5474-5481; or wherein the engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5466-5473. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

In some aspects, the present disclosure provides for an engineered nuclease system comprising: (a) an endonuclease having at least 75% sequence identity to any one of SEQ ID NOs: 215 or a variant thereof, and (b) an engineered guide RNA, wherein the engineered guide RNA is configured to form a complex with the endonuclease and the engineered guide RNA comprises a spacer sequence configured to hybridize to a target nucleic acid sequence, wherein the system has reduced immunogenicity when administered to a human subject compared to an equivalent system comprising a Cas9 enzyme. In some embodiments, the Cas9 enzyme is an SpCas9 enzyme. In some embodiments, the guide RNA comprises a sequence with at least 80% sequence identity to the non-degenerate nucleotides of SEQ ID NO: 3609. In some embodiments, the immunogenicity is antibody immunogenicity.

In some aspects, the present disclosure provides for a method of disrupting a mouse HAO-1 locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the mouse HAO-1 locus, wherein the engineered guide RNA comprises the nucleotides of guide RNAs mH29-1_37, mH29-15_37, mH29-29_37 (SEQ ID NOs: 5779-5781) comprising the nucleotide modifications described in SEQ ID NOs: 5779-5781; or wherein the engineered guide RNA comprises any one of SEQ ID NOs: 4184-4225. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof. In some embodiments, the engineered guide RNA comprises the nucleotides of guide RNAs mH29-15_37 or mH29-29_37 (SEQ ID NOs: 5780-5781) comprising the nucleotide modifications described in SEQ ID NOs: 5780-5781. In some embodiments, the method further comprises disrupting expression of glycolate oxidase from the HAO-1 locus.

In some aspects, the present disclosure provides for a method of disrupting a human TRAC locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the human TRAC locus, wherein the engineered guide RNA comprises the nucleotides of MG29-1-TRAC-sgRNA-35 (SEQ ID NOs: 5681 or 5683) comprising the nucleotide modifications described in SEQ ID NOs: 5681 or 5683. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof.

In some aspects, the present disclosure provides for a method of disrupting an albumin locus in a cell, comprising introducing to the cell: (a) a class 2, type V Cas endonuclease; and (b) an engineered guide RNA, wherein the engineered guide RNA is configured to form a complex with the endonuclease and the engineered guide RNA comprises a spacer sequence configured to hybridize to a region of the albumin locus, wherein the engineered guide RNA comprises the nucleotides of mAlb298-37, mAlb2912-37, mAlb2918-37, or mAlb298-34 (SEQ ID NOs: 5756-5759) comprising the nucleotide modifications described in SEQ ID NOs: 5756-5759; or wherein the engineered guide RNA comprises the nucleotides of mAlb29-8-44, mAlb29-8-50, mAlb29-8-50b, mAlb29-8-51b, mAlb29-8-52b, mAlb29-8-53b, or mAlb29-8-54b comprising the nucleotide modifications described in Table 5. In some embodiments, the class 2, type V Cas endonuclease comprises an endonuclease having at least 75% sequence identity to any one of SEQ ID NOs: 1-3470 or a variant thereof. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036. In some embodiments, the guide RNA comprises a sequence with at least 80% sequence identity to the non-degenerate nucleotides of SEQ ID NO: 3609. In some embodiments, the cell is a eukaryotic cell, hepatocyte, T-cell, hematopoietic stem cell, or precursor thereof. In some embodiments, the engineered guide RNA comprises the nucleotides of mAlb298-37, mAlb2912-37, mAlb2918-37, or mAlb298-34 (SEQ ID NOs: 5756-5759) comprising the nucleotide modifications described in SEQ ID NOs: 5756-5759.

In some aspects, the present disclosure provides for an engineered guide RNA comprising: (a) a DNA-targeting segment comprising a nucleotide sequence that is complementary to a target sequence in a target DNA molecule; and (b) a protein-binding segment configured to bind to a class 2, type V Cas endonuclease, and wherein the guide RNA comprises a nucleotide modification pattern depicted in any one of SEQ ID NOs: 5695-5701. In some embodiments, the guide RNA comprises mAlb29-8-44, mAlb29-8-50, mAlb29-8-37, or mAlb29-12-44. In some embodiments, the guide RNA comprises hH29-4_50, hH29-21_50, hH29-23_50, hH29-41_50, hH29-4_50b, hH29-21_50b, hH29-23_50b, or hH29-41_50b, mH29-1-50, mH29-15-50, mH29-29-50, mH29-1-50b, mH29-15-50b, or mH29-29-50b. In some embodiments, the DNA-targeting segment is configured to hybridize to an HAO-1 gene or an albumin gene. In some embodiments, the class 2, type V Cas endonuclease comprises an endonuclease having at least 75% sequence identity to any one of SEQ ID NOs: 141, 215, 229, 261, or 1711-1722 or a variant thereof. In some embodiments, the class 2, type V Cas endonuclease comprises an endonuclease having at least 75% sequence identity to SEQ ID NO: 215.

In some aspects, the present disclosure provides for an engineered nuclease system comprising: (a) an endonuclease having at least 75% sequence identity to any one of SEQ ID NOS: 1-3470 or a variant thereof, or a nucleotide sequence encoding the endonuclease; and (b) a polynucleotide sequence encoding a CRISPR array, wherein the CRISPR array is configured to be processed by the endonuclease to an engineered guide RNA, wherein the engineered guide RNA is configured to form a complex with the endonuclease and the engineered guide RNA comprises a spacer sequence configured to hybridize to a target nucleic acid sequence, wherein the spacer sequence is configured to hybridize to an albumin gene. In some embodiments, the polynucleotide sequence comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 5712. In some embodiments, the endonuclease comprises an endonuclease having at least 75% sequence identity to any one of SEQ ID NOs: 141, 215, 229, 261, or 1711-1722 or a variant thereof. In some embodiments, the endonuclease comprises an endonuclease having at least 75% sequence identity to SEQ ID NO: 215.

In some aspects, the present disclosure provides for an engineered nuclease system comprising: (a) an endonuclease having at least 75% sequence identity to SEQ ID NOs: 470 or a variant thereof; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the engineered guide RNA comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 6031. In some embodiments, the endonuclease is configured to be selective for a 5′ PAM sequence comprising SEQ ID NO: 6032.

In some aspects, the present disclosure provides for an engineered nuclease system comprising: (a) an endonuclease having at least at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 2824, 2841, or 2896, or a variant thereof; and (b) an engineered guide RNA, wherein the engineered guide RNA is configured to form a complex with the endonuclease and the engineered guide RNA comprises a spacer sequence configured to hybridize to a target nucleic acid sequence, wherein the engineered guide RNA comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 6033, 6034, or 6035. In some embodiments, the endonuclease has at least 80% sequence identity to SEQ ID NO: 2824 and the engineered guide RNA has at least 80% sequence identity to SEQ ID NO: 6033. In some embodiments, the endonuclease has at least 80% sequence identity to SEQ ID NO: 2841 and the engineered guide RNA has at least 80% sequence identity to SEQ ID NO: 6034. In some embodiments, the endonuclease has at least 80% sequence identity to SEQ ID NO: 2896 and the engineered guide RNA has at least 80% sequence identity to SEQ ID NO: 6035. In some embodiments, the endonuclease is configured to be selective for a 5′ PAM sequence comprising any one of SEQ ID NOs: 6037-6039.

In some aspects, the present disclosure provides for a lipid nanoparticle comprising: (a) any of the endonucleases described herein; (b) any of the engineered guide RNAs described herein: (c) a cationic lipid; (d) a sterol; (e) a neutral lipid; and (f) a PEG-modified lipid. In some embodiments, the cationic lipid comprises C12-200, the sterol comprises cholesterol, the neutral lipid comprises DOPE, or the PEG-modified lipid comprises DMG-PEG2000.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the gene-editing outcomes at the DNA level for human GPR146 in Hep3B cells.

FIG. 2 depicts the gene-editing outcomes at the DNA level for mouse GPR146 in Hepa1-6 cells.

FIG. 3 depicts the gene-editing outcomes at the DNA level for human ANGPTL3 in Hep3B cells.

FIG. 4 depicts the gene-editing outcomes at the DNA level for human GPR146 in primary human hepatocytes.

FIG. 5 depicts the gene-editing outcomes at the DNA level for mouse GPR146 in primary mouse hepatocytes.

FIGS. 6A-6C depict predicted folding for single guide RNA (sgRNA) sequences without spacers (Turner, 2004 model). TracrRNA and repeat sequences are looped with a GAAA tetraloop. The repeat anti-repeat fold is on the 3′ end of each structure (right end circle). For FIG. 6A, tracrRNA sequences for these two candidates were obtained from in silico analyses of intergenic regions suggesting they potentially encoded tracrRNAs. For FIGS. 6B and 6C, tracrRNAs were predicted using covariance models built from previously active sgRNAs. The sgRNA number (sg #) is shown below the nuclease number.

FIGS. 7A and 7B depict in vitro cleavage assay amplification products. FIG. 7A depicts 2% agarose gels with low molecular weight DNA ladders (NEB) in the leftmost lanes. FIG. 7B depicts a digital gel from an Agilent Technologies 4200 TapeStation and D1000 ScreenTape System. Resulting amplicon products are 188 bp with a U67 spacer carrying guide or 205 bp with a U40 spacer carrying guide. The specific sgRNA (sg #) and spacer are shown on top of the lanes used for each nuclease.

FIGS. 8A and 8B depict Seq Logos of protospacer adjacent motif (PAM) sequences obtained from NGS sequencing of the amplified cut site on the template strand (FIG. 8A) and the non-template strand (FIG. 8B). The specific sgRNA (sg #) and spacer are shown next to the nuclease number.

FIGS. 9A and 9B depict histograms of the number of DNA reads mapping to each MG91 nuclease's amplified cut site on the template strand (FIG. 9A) and the non-template strand (FIG. 9B). The specific sgRNA (sg #) and spacer are shown next to the nuclease number.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG11

SEQ ID NOs: 1-37 show the full-length peptide sequences of MG11 nucleases.

SEQ ID NO: 3471 shows a crRNA 5′ direct repeats designed to function with an MG11 nuclease.

SEQ ID NOs: 3472-3538 show effector repeat motifs of MG11 nucleases.

SEQ ID NOs: 38-118 show the full-length peptide sequences of MG13 nucleases.

SEQ ID NO: 3540-3550 show effector repeat motifs of MG13 nucleases.

### MG19

SEQ ID NOs: 119-124 show the full-length peptide sequences of MG19 nucleases.

SEQ ID NOs: 3551-3558 show the nucleotide sequences of sgRNAs engineered to function with a MG19 nuclease.

SEQ ID NOs: 3863-3866 show PAM sequences compatible with MG19 nucleases.

### MG20

SEQ ID NO: 125 shows the full-length peptide sequence of a MG20 nuclease.

SEQ ID NO: 3559 shows the nucleotide sequence of a sgRNA engineered to function with a MG20 nuclease.

SEQ ID NO: 3867 shows a PAM sequence compatible with an MG20 nuclease.

### MG26

SEQ ID NOs: 126-140 show the full-length peptide sequences of MG26 nucleases.

SEQ ID NOs: 3560-3572 show effector repeat motifs of MG26 nucleases.

### MG28

SEQ ID NOs: 141-214 show the full-length peptide sequences of MG28 nucleases.

SEQ ID NOs: 3573-3607 show effector repeat motifs of MG28 nucleases.

SEQ ID NOs: 3608-3609 show crRNA 5′ direct repeats designed to function with an MG28 nuclease.

SEQ ID NOs: 3868-3869 shows a PAM sequence compatible with an MG28 nuclease.

### MG29

SEQ ID NOs: 215-225 and 6340-6551 show the full-length peptide sequences of MG29 nucleases.

SEQ ID NO: 5680 shows the nucleotide sequence of an MG29-1 nuclease containing 5′ UTR, NLS, CDS, NLS, 3′ UTR, and poly A tail.

SEQ ID NOs: 3610-3611 show effector repeat motifs of MG29 nucleases.

SEQ ID NO: 3612 shows the nucleotide sequence of a sgRNA engineered to function with a MG29 nuclease.

SEQ ID NOs: 3870-3872 show PAM sequences compatible with an MG29 nuclease.

SEQ ID NO: 5687 shows an MG29-1 coding sequence used for the generation of mRNA.

SEQ ID NOs: 5830 and 5846 show DNA sequences encoding MG29-1 mRNAs.

### MG30

SEQ ID NOs: 226-228 show the full-length peptide sequences of MG30 nucleases.

SEQ ID NOs: 3613-3615 show effector repeat motifs of MG30 nucleases.

SEQ ID NO: 3873 shows a PAM sequence compatible with an MG30 nuclease.

### MG31

SEQ ID NOs: 229-260 show the full-length peptide sequences of MG31 nucleases.

SEQ ID NOs: 3616-3632 show effector repeat motifs of MG31 nucleases.

SEQ ID NOs: 3874-3876 show PAM sequences compatible with a MG31 nuclease.

### MG32

SEQ ID NO: 261 shows the full-length peptide sequence of a MG32 nuclease.

SEQ ID NO: 3633-3634 show effector repeat motifs of MG32 nucleases.

SEQ ID NO: 3876 shows a PAM sequence compatible with a MG32 nuclease.

### MG37

SEQ ID NOs: 262-426 show the full-length peptide sequences of MG37 nucleases.

SEQ ID NO: 3635 shows an effector repeat motif of MG37 nucleases.

SEQ ID NOs: 3636-3637, 3640-3641, 3644-3645, 3648-3649, 3652-3653, 3656-3657, and 3660-3661 show the nucleotide sequence of sgRNA engineered to function with an MG37 nuclease.

SEQ ID NOs: 3638, 3642, 3646, 3650, 3654, 3658, and 3662 show the nucleotide sequences of MG37 tracrRNAs derived from the same loci as MG37 nucleases above.

SEQ ID NO: 3639, 3643, 3647, 3651, 3655, and 3659 show 5′ direct repeat sequences derived from native MG37 loci that serve as crRNAs when placed 5′ to a 3′ targeting or spacer sequence.

### MG53

SEQ ID NOs: 427-428 show the full-length peptide sequences of MG53 nucleases.

SEQ ID NO: 3663 shows a 5′ direct repeat sequence derived from native MG53 loci that serve as a crRNA when placed 5′ to a 3′ targeting or spacer sequence.

SEQ ID NOs: 3664-3667 show the nucleotide sequence of sgRNAs engineered to function with an MG53 nuclease.

SEQ ID NOs: 3668-3669 show the nucleotide sequences of MG53 tracrRNAs derived from the same loci as MG53 nucleases above.

### MG54

SEQ ID NOs: 429-430 show the full-length peptide sequences of MG54 nucleases.

SEQ ID NO: 3670 shows a 5′ direct repeat sequence derived from native MG54 loci that serve as a crRNA when placed 5′ to a 3′ targeting or spacer sequence.

SEQ ID NOs: 3671-3672 show the nucleotide sequence of sgRNA engineered to function with an MG54 nuclease.

SEQ ID NOs: 3673-3676 show the nucleotide sequences of MG54 tracrRNAs derived from the same loci as MG54 nucleases above.

### MG55

SEQ ID NOs: 431-688 show the full-length peptide sequences of MG55 nucleases.

SEQ ID NO: 6031 shows the nucleotide sequence of an sgRNA engineered to function with an MG55 nuclease.

SEQ ID NO: 6032 shows a PAM sequence compatible with an MG55 nuclease.

### MG56

SEQ ID NOs: 689-690 show the full-length peptide sequences of MG56 nucleases.

SEQ ID NO: 3678 shows a crRNA 5′ direct repeats designed to function with an MG56 nuclease.

SEQ ID NOs: 3679-3680 show effector repeat motifs of MG56 nucleases.

### MG57

SEQ ID NOs: 691-721 show the full-length peptide sequences of MG57 nucleases.

SEQ ID NOs: 3681-3694 show effector repeat motifs of MG57 nucleases.

SEQ ID NOs: 3695-3696 show the nucleotide sequences of sgRNAs engineered to function with an MG57 nuclease.

SEQ ID NOs: 3879-3880 shows PAM sequences compatible with MG57 nucleases.

### MG58

SEQ ID NOs: 722-779 show the full-length peptide sequences of MG58 nucleases.

SEQ ID NOs: 3697-3711 show effector repeat motifs of MG58 nucleases.

### MG59

SEQ ID NOs: 780-792 show the full-length peptide sequences of MG59 nucleases.

SEQ ID NOs: 3712-3728 show effector repeat motifs of MG59 nucleases.

SEQ ID NOs: 3729-3730 show the nucleotide sequences of sgRNAs engineered to function with an MG59 nuclease.

SEQ ID NOs: 3881-3882 shows PAM sequences compatible with MG59 nucleases.

### MG60

SEQ ID NOs: 793-1163 show the full-length peptide sequences of MG60 nucleases.

SEQ ID NOs: 3731-3733 show effector repeat motifs of MG60 nucleases.

### MG61

SEQ ID NOs: 1164-1469 show the full-length peptide sequences of MG61 nucleases.

SEQ ID NOs: 3734-3735 show crRNA 5′ direct repeats designed to function with MG61 nucleases.

SEQ ID NOs: 3736-3847 show effector repeat motifs of MG61 nucleases.

### MG62

SEQ ID NOs: 1470-1472 show the full-length peptide sequences of MG62 nucleases.

SEQ ID NOs: 3848-3850 show effector repeat motifs of MG62 nucleases.

### MG70

SEQ ID NOs: 1473-1514 show the full-length peptide sequences of MG70 nucleases.

### MG75

SEQ ID NOs: 1515-1710 show the full-length peptide sequences of MG75 nucleases.

### MG77

SEQ ID NOs: 1711-1712 show the full-length peptide sequences of MG77 nucleases.

SEQ ID NOs: 3851-3852 show the nucleotide sequences of sgRNAs engineered to function with an MG77 nuclease.

SEQ ID NOs: 3883-3884 show PAM sequences compatible with MG77 nucleases.

### MG78

SEQ ID NOs: 1713-1717 show the full-length peptide sequences of MG78 nucleases.

SEQ ID NO: 3853 shows the nucleotide sequence of a sgRNA engineered to function with an MG78 nuclease.

SEQ ID NO: 3885 shows a PAM sequence compatible with a MG78 nuclease.

### MG79

SEQ ID NOs: 1718-1722 show the full-length peptide sequences of MG79 nucleases.

SEQ ID NOs: 3854-3857 shows the nucleotide sequences of sgRNAs engineered to function with an MG79 nuclease.

SEQ ID NOs: 3886-3889 show the PAM sequences compatible with MG79 nucleases.

### MG80

SEQ ID NO: 1723 shows the full-length peptide sequence of a MG80 nuclease.

### MG81

SEQ ID NOs: 1724-2654 show the full-length peptide sequences of MG81 nucleases.

### MG82

SEQ ID NOs: 2655-2657 show the full-length peptide sequences of MG82 nucleases.

### MG83

SEQ ID NOs: 2658-2659 show the full-length peptide sequences of MG83 nucleases.

### MG84

SEQ ID NOs: 2660-2677 show the full-length peptide sequences of MG84 nucleases.

### MG85

SEQ ID NOs: 2678-2680 show the full-length peptide sequences of MG85 nucleases.

### MG90

SEQ ID NOs: 2681-2809 show the full-length peptide sequences of MG90 nucleases.

### MG91

SEQ ID NOs: 2810-3470 and 6274-6281 show the full-length peptide sequences of MG91 nucleases.

SEQ ID NOs: 6033-6036 and 6284-6325 show nucleotide sequences of sgRNAs engineered to function with MG91 nucleases.

SEQ ID NOs: 6037-6039 and 6326-6339 show PAM sequences compatible with MG91 nucleases.

SEQ ID NOs: 6040-6049 and 6282 show MG91 intergenic regions potentially encoding tracrRNAs.

SEQ ID NOs: 6050-6059 and 6283 show MG91 CRISPR repeats.

### Spacer Segments

SEQ ID NOs: 3858-3861 show the nucleotide sequences of spacer segments.

### NLS

SEQ ID NOs: 3938-3953 show the sequences of example nuclear localization sequences (NLSs) that can be appended to nucleases according to the disclosure.

### CD38 Targeting

SEQ ID NOs: 4428-4465 and 5685 show the nucleotide sequences of sgRNAs engineered to function with an MG29-1 nuclease in order to target CD38.

SEQ ID NOs: 4466-4503 and 5686 show the DNA sequences of CD38 target sites.

### TIGIT Targeting

SEQ ID NOs: 4504-4520 show the nucleotide sequences of sgRNAs engineered to function with an MG29-1 nuclease in order to target TIGIT.

SEQ ID NOs: 4521-4537 show the DNA sequences of TIGIT target sites.

### AAVS1 Targeting

SEQ ID NOs: 4538-4568 show the nucleotide sequences of sgRNAs engineered to function with an MG29-1 nuclease in order to target AAVS1.

SEQ ID NOs: 4569-4599 show the DNA sequences of AAVS1 target sites.

### B2M Targeting

SEQ ID NOs: 4600-4675 show the nucleotide sequences of sgRNAs engineered to function with an MG29-1 nuclease in order to target B2M.

SEQ ID NOs: 4676-4751 show the DNA sequences of B2M target sites.

### CD2 Targeting

SEQ ID NOs: 4752-4836 show the nucleotide sequences of sgRNAs engineered to function with an MG29-1 nuclease in order to target CD2.

SEQ ID NOs: 4837-4921 show the DNA sequences of CD2 target sites.

### CD5 Targeting

SEQ ID NOs: 4922-4945 show the nucleotide sequences of sgRNAs engineered to function with an MG29-1 nuclease in order to target CD5.

SEQ ID NOs: 4946-4969 show the DNA sequences of CD5 target sites.

hRosa26 Targeting

SEQ ID NOs: 4970-5012 show the nucleotide sequences of sgRNAs engineered to function with an MG29-1 nuclease in order to target hRosa26.

SEQ ID NOs: 5013-5055 show the DNA sequences of hRosa26 target sites.

### TRAC Targeting

SEQ ID NOs: 5056-5125, 5681, and 5683 show the nucleotide sequences of sgRNAs engineered to function with an MG29-1 nuclease in order to target TRAC.

SEQ ID NOs: 5126-5195, 5682, and 5684 show the DNA sequences of TRAC target sites.

### TRBC1 Targeting

SEQ ID NOs: 5196-5210 show the nucleotide sequences of sgRNAs engineered to function with an MG29-1 nuclease in order to target TRBC1.

SEQ ID NOs: 5211-5225 show the DNA sequences of TRBC1 target sites.

### TRBC2 Targeting

SEQ ID NOs: 5226-5246 show the nucleotide sequences of sgRNAs engineered to function with an MG29-1 nuclease in order to target TRBC2.

SEQ ID NOs: 5247-5267 show the DNA sequences of TRBC2 target sites.

### TRBC1/2 Targeting

SEQ ID NOs: 5642-5660 show the nucleotide sequences of sgRNAs engineered to function with an MG29-1 nuclease in order to target TRBC.

SEQ ID NOs: 5661-5679 show the DNA sequences of TRBC target sites.

### FAS Targeting

SEQ ID NOs: 5268-5366 show the nucleotide sequences of sgRNAs engineered to function with an MG29-1 nuclease in order to target FAS.

SEQ ID NOs: 5367-5465 show the DNA sequences of FAS target sites.

### PD-1 Targeting

SEQ ID NOs: 5466-5473 show the nucleotide sequences of sgRNAs engineered to function with an MG29-1 nuclease in order to target PD-1.

SEQ ID NOs: 5474-5481 show the DNA sequences of PD-1 target sites.

### HPRT Targeting

SEQ ID NOs: 5482-5561 show the nucleotide sequences of sgRNAs engineered to function with an MG29-1 nuclease in order to target HPRT.

SEQ ID NOs: 5562-5641 show the DNA sequences of HPRT target sites.

### HAO-1 Targeting

SEQ ID NOs: 5788-5829 and 5831-5834 show the nucleotide sequences of sgRNAs engineered to function with an MG29-1 nuclease in order to target human HAO-1.

SEQ ID NOs: 5836-5845 show the nucleotide sequences of sgRNAs engineered to function with an MG29-1 nuclease in order to target mouse HAO-1.

### APO-A1 Targeting

SEQ ID NOs: 5847-5860 show the nucleotide sequences of sgRNAs engineered to function with an MG29-1 nuclease in order to target mouse APO-A1.

SEQ ID NOs: 5861-5874 show the DNA sequences of APO-A1 target sites.

### Mouse ANGPTL3 Targeting

SEQ ID NOs: 5875-5952 show the nucleotide sequences of sgRNAs engineered to function with an MG29-1 nuclease in order to target mouse ANGPTL3.

SEQ ID NOs: 5953-6030 show the DNA sequences of mouse ANGPTL3 target sites.

### MG29-1 Human GPR146 Targeting

SEQ ID NOs: 6060-6068 show the nucleotide sequences of sgRNAs engineered to function with an MG29-1 nuclease in order to target human GPR146.

SEQ ID NOs: 6069-6077 show the DNA sequences of human GPR146 target sites.

### MG29-1 Mouse GPR146 Targeting

SEQ ID NOs: 6078-6079 show the nucleotide sequences of sgRNAs engineered to function with an MG29-1 nuclease in order to target mouse GPR146.

SEQ ID NOs: 6080-6081 show the DNA sequences of mouse GPR146 target sites.

### Human ANGPTL3 Targeting

SEQ ID NOs: 6082-6177 show the nucleotide sequences of sgRNAs engineered to function with an MG29-1 nuclease in order to target human ANGPTL3.

SEQ ID NOs: 6178-6273 show the DNA sequences of human ANGPTL3 target sites.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Il.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. Eukaryotic basal promoters can contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” can generally be used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “Cas12a” generally refers to a family of Cas endonucleases that are class 2, Type V-A Cas endonucleases and that (a) use a relatively small guide RNA (about 42-44 nucleotides) that is processed by the nuclease itself following transcription from the CRISPR array, and (b) cleave DNA to leave staggered cut sites. Further features of this family of enzymes can be found, e.g. in Zetsche B, Heidenreich M, Mohanraju P, et al. Nat Biotechnol 2017; 35:31-34, and Zetsche B, Gootenberg J S, Abudayyeh O O, et al. Cell 2015; 163:759-771, which are incorporated by reference herein.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% sequence identity to any one of the endonuclease protein sequences described herein (e.g. MG11, MG13, MG26, MG28, MG29, MG30, MG31, MG32, MG37, MG53, MG54, MG55, MG56, MG57, MG58, MG59, MG60, MG61, MG62, MG70, MG82, MG83, MG84 or MG85 family endonucleases described herein, or any other family nuclease described herein). In some embodiments, such conservatively substituted variants are functional variants. Such functional variants can encompass sequences with substitutions such that the activity of one or more critical active site residues or guide RNA binding residues of the endonuclease are not disrupted. In some embodiments, a functional variant of any of the proteins described herein lacks substitution of at least one conserved or functional residue. In some embodiments, a functional variant of any of the proteins described herein lacks substitution of all conserved or functional residues.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered

Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches containing large numbers of microbial species may offer the potential to drastically increase the number of new CRISPR/Cas systems characterized and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity.

Class I CRISPR-Cas systems have large, multi-subunit effector complexes, and comprise Types I, III, and IV. Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are identified as DNA nucleases. Type 2 effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again identified as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

CRISPR-Cas systems have emerged in recent years as the gene editing technology of choice due to their targetability and ease of use. The most commonly used systems are the Class 2 Type II SpCas9 and the Class 2 Type V-A Cas12a. The Type V-A systems in particular are becoming more widely used since their reported specificity in cells is higher than other nucleases, with fewer or no off-target effects. The V-A systems are also advantageous in that the guide RNA is small (42-44 nucleotides compared with approximately 100 nt for SpCas9) and is processed by the nuclease itself following transcription from the CRISPR array, simplifying multiplexed applications with multiple gene edits. Furthermore, the V-A systems have staggered cut sites, which may facilitate directed repair pathways, such as microhomology-dependent targeted integration (MITI).

The most commonly used Type V-A enzymes require a 5′ protospacer adjacent motif (PAM) next to the chosen target site: 5′-TTTV-3′ for Lachnospiraceae bacterium ND2006 LbCas12a and Acidaminococcus sp. AsCas12a; and 5′-TTV-3′ for Francisella novicida FnCas12a. Recent exploration of orthologs has revealed proteins with less restrictive PAM sequences that are also active in mammalian cell culture, for example YTV, YYN or TTN. However, these enzymes do not fully encompass V-A biodiversity and targetability, and may not represent all possible activities and PAM sequence requirements. Here, thousands of genomic fragments were mined from numerous metagenomes for Type V-A nucleases. The diversity of identified V-A enzymes may have been expanded and novel systems may have been developed into highly targetable, compact, and precise gene editing agents.

### MG Enzymes

Type V-A CRISPR systems are quickly being adopted for use in a variety of genome editing applications. These programmable nucleases are part of adaptive microbial immune systems, the natural diversity of which has been largely unexplored. Novel families of Type V-A CRISPR enzymes were identified through a large-scale analysis of metagenomes collected from a variety of complex environments, and developed representatives of these systems into gene-editing platforms. The nucleases are phylogenetically diverse and recognize a single guide RNA with specific motifs. The majority of these systems come from uncultivated organisms, some of which encode a divergent Type V effector within the same CRISPR operon. Biochemical analysis uncovered unexpected PAM diversity, indicating that these systems will facilitate a variety of genome engineering applications. The simplicity of guide sequences and activity in human cell lines suggest utility in gene and cell therapies.

In some aspects, the present disclosure provides for novel Type V-L candidates. Type V-L may be a novel subtype and some sub-families may have been identified. These nucleases are about 1000-1100 amino acids in length. Type V-L may be found in the same CRISPR locus as Type V-A effectors. RuvC catalytic residues may have been identified for Type V-L candidates and these Type V-L candidates may not require tracrRNA. One example of a Type V-L are the MG60 nucleases described herein.

In some aspects, the present disclosure provides for smaller Type V effectors. Such effectors may be small putative effectors. These effectors may simplify delivery and may extend therapeutic applications.

In some aspects, the present disclosure provides for novel type V effector. Such an effector may be MG70 as described herein. MG70 may be an ultra-small enzyme of about 373 amino acids in length. MG 70 may have a single transposase domain at the N-terminus and may have a predicted tracrRNA.

In some aspects, the present disclosure provides for a smaller Type V effector. Such an effector may be MG81 described herein. MG81 may be about 500-700 amino acids in length and may contain RuvC, and HTH DNA binding domains.

In one aspect, the present disclosure provides for an engineered nuclease system discovered through metagenomic sequencing. In some cases, the metagenomic sequencing is conducted on samples. In some cases, the samples may be collected from a variety of environments. Such environments may be a human microbiome, an animal microbiome, environments with high temperatures, environments with low temperatures. Such environments may include sediment.

In one aspect, the present disclosure provides for an engineered nuclease system comprising (a) an endonuclease. In some cases, the endonuclease is a Cas endonuclease. In some cases, the endonuclease is a class 2, type V Cas endonuclease. In some cases, the endonuclease is a class 2, type V-A Cas endonuclease. In some cases, the endonuclease is derived from an uncultivated microorganism. The endonuclease may comprise a RuvC domain. In some cases, the engineered nuclease system comprises (b) an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In one aspect, the present disclosure provides for an engineered nuclease system comprising (a) an endonuclease. In some cases, the endonuclease has at least about 70% sequence identity to any one of SEQ ID NOs: 1-3470. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-3470.

In some cases, the endonuclease comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-3470. In some cases, the endonuclease may be substantially identical to any one of SEQ ID NOs: 1-3470.

In some cases, the engineered nuclease system comprises an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In one aspect, the present disclosure provides an engineered nuclease system comprising (a) an endonuclease. In some cases, the endonuclease is configured to bind to a protospacer adjacent motif (PAM) sequence. In some cases, the PAM sequence is substantially identical to any one of SEQ ID NOs: 3863-3913. In some cases, the PAM sequence any one of SEQ ID NOs: 3863-3913. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class 2, type V-A Cas endonuclease. In some cases, the engineered nuclease system comprises (b) an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In some cases, the endonuclease is not a Cpf1 or Cms1 endonuclease. In some cases, the endonuclease further comprises a zinc finger-like domain.

In some cases, the guide RNA comprises a sequence with at least 80% sequence identity to the first 19 nucleotides or the non-degenerate nucleotides of SEQ ID NO: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, or 3851-3857. In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, or 3851-3857. In some cases, the guide RNA comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, or 3851-3857. In some cases, the guide RNA comprises a sequence which is substantially identical to the first 19 nucleotides or the non-degenerate nucleotides of SEQ ID NO: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, or 3851-3857.

In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, or 3851-3857. In some cases, the endonuclease is configured to bind to the engineered guide RNA. In some cases, the Cas endonuclease is configured to bind to the engineered guide RNA. In some cases, the class 2 Cas endonuclease is configured to bind to the engineered guide RNA. In some cases, the class 2, type V Cas endonuclease is configured to bind to the engineered guide RNA. In some cases, the class 2, type V-A Cas endonuclease is configured to bind to the engineered guide RNA.

In some cases, the endonuclease is configured to bind to a protospacer adjacent motif (PAM) sequence comprising any one of SEQ ID NOs: 3863-3913.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some cases, the guide RNA is 30-250 nucleotides in length. In some cases, the guide RNA is 42-44 nucleotides in length. In some cases, the guide RNA is 42 nucleotides in length. In some cases, the guide RNA is 43 nucleotides in length. In some cases, the guide RNA is 44 nucleotides in length. In some cases, the guide RNA is 85-245 nucleotides in length. In some cases, the guide RNA is more than 90 nucleotides in length. In some cases, the guide RNA is less than 245 nucleotides in length.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOs: 3938-3953, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3938-3953. In some cases, the NLS may comprise a sequence substantially identical to any one of SEQ ID NOs: 3938-3953.

TABLE 1
Example NLS Sequences that may be used with
Cas Effectors according to the disclosure.

| | NLS amino acid | SEQ ID |
|---|---|---|
| Source | sequence | NO: |
| SV40 | PKKKRKV | 3938 |
| nucleoplasmin | KRPAATKKAGQAKKKK | 3939 |
| bipartite NLS | | |
| c-myc NLS | PAAKRVKLD | 3940 |
| c-myc NLS | RQRRNELKRSP | 3941 |
| hRNPA1 M9 NLS | NQSSNFGPMKGGNFGGRSSGP | 3942 |
| | YGGGGQYFAKPRNQGGY | |
| Importin-alpha IBB | RMRIZFKNKGKDTAELRRRRV | 3943 |
| domain | EVSVELRKAKKDEQILKRRNV | |
| Myoma T protein | VSRKRPRP | 3944 |
| Myoma T protein | PPKKARED | 3945 |
| p53 | PQPKKKPL | 3946 |
| mouse c-abl IV | SALIKKKKKMAP | 3947 |
| influenza virus NS1 | DRLRR | 3948 |
| influenza virus NS1 | PKQKKRK | 3949 |
| Hepatitis virus delta | RKLKKKIKKL | 3950 |
| antigen | | |
| mouse Mx1 protein | REKKKFLKRR | 3951 |
| human poly (ADP- | KRKGDEVDGVDEVAKKKSKK | 3952 |
| ribose) polymerase | | |
| steroid hormonc | RKCLQAGMNLEARKTKK | 3953 |
| receptors (human) | | |
| glucocorticoid | | |

In some cases, the engineered nuclease system further comprises a single- or double stranded DNA repair template. In some cases, the engineered nuclease system further comprises a single-stranded DNA repair template. In some cases, the engineered nuclease system further comprises a double-stranded DNA repair template. In some cases, the single- or double-stranded DNA repair template may comprise from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.

In some cases, the first homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides. In some cases, the second homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides.

In some cases, the first and second homology arms are homologous to a genomic sequence of a prokaryote. In some cases, the first and second homology arms are homologous to a genomic sequence of a bacteria. In some cases, the first and second homology arms are homologous to a genomic sequence of a fungus. In some cases, the first and second homology arms are homologous to a genomic sequence of a eukaryote.

In some cases, the engineered nuclease system further comprises a DNA repair template. The DNA repair template may comprise a double-stranded DNA segment. The double-stranded DNA segment may be flanked by one single-stranded DNA segment. The double-stranded DNA segment may be flanked by two single-stranded DNA segments. In some cases, the single-stranded DNA segments are conjugated to the 5′ ends of the double-stranded DNA segment. In some cases, the single stranded DNA segments are conjugated to the 3′ ends of the double-stranded DNA segment.

In some cases, the single-stranded DNA segments have a length from 1 to 15 nucleotide bases. In some cases, the single-stranded DNA segments have a length from 4 to 10 nucleotide bases. In some cases, the single-stranded DNA segments have a length of 4 nucleotide bases. In some cases, the single-stranded DNA segments have a length of 5 nucleotide bases. In some cases, the single-stranded DNA segments have a length of 6 nucleotide bases. In some cases, the single-stranded DNA segments have a length of 7 nucleotide bases. In some cases, the single-stranded DNA segments have a length of 8 nucleotide bases. In some cases, the single-stranded DNA segments have a length of 9 nucleotide bases. In some cases, the single-stranded DNA segments have a length of 10 nucleotide bases.

In some cases, the single-stranded DNA segments have a nucleotide sequence complementary to a sequence within the spacer sequence. In some cases, the double-stranded DNA sequence comprises a barcode, an open reading frame, an enhancer, a promoter, a protein-coding sequence, a miRNA coding sequence, an RNA coding sequence, or a transgene.

In some cases, the engineered nuclease system further comprises a source of Mg²⁺.

In some cases, the guide RNA comprises a hairpin comprising at least 8 base-paired ribonucleotides. In some cases, the guide RNA comprises a hairpin comprising at least 9 base-paired ribonucleotides. In some cases, the guide RNA comprises a hairpin comprising at least 10 base-paired ribonucleotides. In some cases, the guide RNA comprises a hairpin comprising at least 11 base-paired ribonucleotides. In some cases, the guide RNA comprises a hairpin comprising at least 12 base-paired ribonucleotides.

In some cases, the endonuclease comprises a sequence at least 70% identical to a variant of any one of SEQ ID NOs: 141, 215, 229, 261, or 1711-1721 or a variant thereof 141, 215, 229, 261, or 1711-1721 or a variant thereof. In some cases, the endonuclease comprises a sequence at least 75% identical to a variant of any one of SEQ ID NOs: 141, 215, 229, 261, or 1711-1721 or a variant thereof. In some cases, the endonuclease comprises a sequence at least 80% identical to a variant of any one of SEQ ID NOs: 141, 215, 229, 261, or 1711-1721 or a variant thereof. In some cases, the endonuclease comprises a sequence at least 85% identical to a variant of any one of SEQ ID NOS: 141, 215, 229, 261, or 1711-1721 or a variant thereof. In some cases, the endonuclease comprises a sequence at least 90% identical to a variant of any one of SEQ ID NOs: 141, 215, 229, 261, or 1711-1721 or a variant thereof. In some cases, the endonuclease comprises a sequence at least 95% identical to a variant of any one of SEQ ID NOs: 141, 215, 229, 261, or 1711-1721 or a variant thereof

In some cases, the guide RNA structure comprises a sequence of at least 70% identical to the first 19 nucleotides or the non-degenerate nucleotides of SEQ ID NO: 3608. In some cases, the guide RNA structure comprises a sequence of at least 75% identical to the first 19 nucleotides or the non-degenerate nucleotides of SEQ ID NO: 3608. In some cases, the guide RNA structure comprises a sequence of at least 80% identical to the first 19 nucleotides or the non-degenerate nucleotides of SEQ ID NO: 3608. In some cases, the guide RNA structure comprises a sequence of at least 85% identical to the first 19 nucleotides or the non-degenerate nucleotides of SEQ ID NO: 3608. In some cases, the guide RNA structure comprises a sequence of at least 90% identical to the first 19 nucleotides or the non-degenerate nucleotides of SEQ ID NO: 3608. In some cases, the guide RNA structure comprises a sequence of at least 95% identical to the first 19 nucleotides or the non-degenerate nucleotides of SEQ ID NO: 3608. In some cases, the endonuclease is configured to bind to a PAM comprising any one of SEQ ID NOs: 3863-3913.

In some cases, sequence may be determined by a BLASTP, CLUSTALW, MUSCLE, or MAFFT algorithm, or a CLUSTALW algorithm with the Smith-Waterman homology search algorithm parameters. The sequence identity may be determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In one aspect, the present disclosure provides an engineered guide RNA comprising (a) a DNA-targeting segment. In some cases, the DNA-targeting segment comprises a nucleotide sequence that is complementary to a target sequence. In some cases, the target sequence is in a target DNA molecule. In some cases, the engineered guide RNA comprises (b) a protein-binding segment. In some cases, the protein-binding segment comprises two complementary stretches of nucleotides. In some cases, the two complementary stretches of nucleotides hybridize to form a double-stranded RNA (dsRNA) duplex. In some cases, the two complementary stretches of nucleotides are covalently linked to one another with intervening nucleotides. In some cases, the engineered guide ribonucleic acid polynucleotide is capable of forming a complex with an endonuclease. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-3470. In some cases, the complex targets the target sequence of the target DNA molecule.

In some cases, the DNA-targeting segment is positioned 3′ of both of the two complementary stretches of nucleotides. In some cases, the protein binding segment comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 3608.

In some cases, the double-stranded RNA (dsRNA) duplex comprises at least 8 ribonucleotides. In some cases, the double-stranded RNA (dsRNA) duplex comprises at least 9 ribonucleotides. In some cases, the double-stranded RNA (dsRNA) duplex comprises at least 10 ribonucleotides. In some cases, the double-stranded RNA (dsRNA) duplex comprises at least 11 ribonucleotides. In some cases, the double-stranded RNA (dsRNA) duplex comprises at least 12 ribonucleotides.

In some cases, the deoxyribonucleic acid polynucleotide encodes the engineered guide ribonucleic acid polynucleotide.

In one aspect, the present disclosure provides a nucleic acid comprising an engineered nucleic acid sequence. In some cases, the engineered nucleic acid sequence is optimized for expression in an organism. In some cases, the nucleic acid encodes an endonuclease. In some cases, the endonuclease is a Cas endonuclease. In some cases, the endonuclease is a class 2 endonuclease. In some cases, the endonuclease is a class2, type V Cas endonuclease. In some cases, the endonuclease is a class2, type V-A Cas endonuclease. In some cases, the endonuclease is derived from an uncultivated microorganism. In some cases, the organism is not the uncultivated organism.

In some cases, the endonuclease comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-3470.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOs: 3938-3953, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 3938-3953.

In some cases, the organism is prokaryotic. In some cases, the organism is bacterial. In some cases, the organism is eukaryotic. In some cases, the organism is fungal. In some cases, the organism is a plant. In some cases, the organism is mammalian. In some cases, the organism is a rodent. In some cases, the organism is human.

In one aspect, the present disclosure provides an engineered vector. In some cases, the engineered vector comprises a nucleic acid sequence encoding an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V-A Cas endonuclease. In some cases, the endonuclease is derived from an uncultivated microorganism.

In some cases, the engineered vector comprises a nucleic acid described herein. In some cases, the nucleic acid described herein is a deoxyribonucleic acid polynucleotide described herein. In some cases, the vector is a plasmid, a minicircle, a CELiD, an adeno-associated virus (AAV) derived virion, or a lentivirus.

In one aspect, the present disclosure provides a cell comprising a vector described herein.

In one aspect, the present disclosure provides a method of manufacturing an endonuclease. In some cases, the method comprises cultivating the cell.

In one aspect, the present disclosure provides a method for binding, cleaving, marking, or modifying a double-stranded deoxyribonucleic acid polynucleotide. The method may comprise contacting the double-stranded deoxyribonucleic acid polynucleotide with an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V-A Cas endonuclease. In some cases, the endonuclease is in complex with an engineered guide RNA. In some cases, the engineered guide RNA is configured to bind to the endonuclease. In some cases, the engineered guide RNA is configured to bind to the double-stranded deoxyribonucleic acid polynucleotide. In some cases, the engineered guide RNA is configured to bind to the endonuclease and to the double-stranded deoxyribonucleic acid polynucleotide. In some cases, the double-stranded deoxyribonucleic acid polynucleotide comprises a protospacer adjacent motif (PAM). In some cases, the PAM comprises a sequence comprising any one of SEQ ID NOs: 3863-3913.

In some cases, the double-stranded deoxyribonucleic acid polynucleotide comprises a first strand comprising a sequence complementary to a sequence of the engineered guide RNA and a second strand comprising the PAM. In some cases, the PAM is directly adjacent to the 5′ end of the sequence complementary to the sequence of the engineered guide RNA. In some cases, the endonuclease is not a Cpf1 endonuclease or a Cms1 endonuclease. In some cases, the endonuclease is derived from an uncultivated microorganism. In some cases, the double-stranded deoxyribonucleic acid polynucleotide is a eukaryotic, plant, fungal, mammalian, rodent, or human double-stranded deoxyribonucleic acid polynucleotide. In some cases, the PAM comprises any one of SEQ ID NOs: 3863-3913.

In one aspect, the present disclosure provides a method of modifying a target nucleic acid locus. The method may comprise delivering to the target nucleic acid locus the engineered nuclease system described herein. In some cases, the endonuclease is configured to form a complex with the engineered guide ribonucleic acid structure. In some cases, the complex is configured such that upon binding of the complex to the target nucleic acid locus, the complex modifies the target nucleic acid locus.

In some cases, modifying the target nucleic acid locus comprises binding, nicking, cleaving, or marking said target nucleic acid locus. In some cases, the target nucleic acid locus comprises deoxyribonucleic acid (DNA) or ribonucleic acid (RNA). In some cases, the target nucleic acid comprises genomic DNA, viral DNA, viral RNA, or bacterial DNA. In some cases, the target nucleic acid locus is in vitro. In some cases, the target nucleic acid locus is within a cell. In some cases, the cell is a prokaryotic cell, a bacterial cell, a eukaryotic cell, a fungal cell, a plant cell, an animal cell, a mammalian cell, a rodent cell, a primate cell, or a human cell.

In some cases, delivery of the engineered nuclease system to the target nucleic acid locus comprises delivering the nucleic acid described herein or the vector described herein. In some cases, delivery of engineered nuclease system to the target nucleic acid locus comprises delivering a nucleic acid comprising an open reading frame encoding the endonuclease. In some cases, the nucleic acid comprises a promoter. In some cases, the open reading frame encoding the endonuclease is operably linked to the promoter.

In some cases, delivery of the engineered nuclease system to the target nucleic acid locus comprises delivering a capped mRNA containing the open reading frame encoding the endonuclease. In some cases, delivery of the engineered nuclease system to the target nucleic acid locus comprises delivering a translated polypeptide. In some cases, delivery of the engineered nuclease system to the target nucleic acid locus comprises delivering a deoxyribonucleic acid (DNA) encoding the engineered guide RNA operably linked to a ribonucleic acid (RNA) pol III promoter.

In some cases, the endonuclease induces a single-stranded break or a double-stranded break at or proximal to the target locus. In some cases, the endonuclease induces a staggered single stranded break within or 3′ to said target locus.

In some cases, effector repeat motifs are used to inform guide design of MG nucleases. For example, the processed gRNA in Type V-A systems comprises the last 20-22 nucleotides of a CRISPR repeat. This sequence may be synthesized into a crRNA (along with a spacer) and tested in vitro, along with the synthesized nucleases, for cleavage on a library of possible targets. Using this method, the PAM may be determined. In some cases, Type V-A enzymes may use a “universal” gRNA. In some cases, Type V enzymes may utilize a unique gRNA.

### Lipid Nanoparticles

Lipid nanoparticles as described herein can be 4-component lipid nanoparticles. Such nanoparticles can be configured for delivery of RNA or other nucleic acids (e.g. synthetic RNA, mRNA, or in vitro-synthesized mRNA) and can be generally formulated as described in WO2012135805A2, which is incorporated by reference herein for all purposes. Such nanoparticles can generally comprise: (a) a cationic lipid (e.g. 98N12-5 (TETA5-LAP), DLin DMA, DLin-K-DMA (2,2-Dilinoleyl-4-dimethylaminomethyl-[1,3]-dioxolane), DLin-KC2-DMA, DLin-MC3-DMA, or C12-200), (b) a neutral lipid (e.g. DSPC or DOPE), (c) a sterol (e.g. cholesterol or a cholesterol analog), and (d) a PEG-modified lipid (e.g. PEG-DMG).

The cationic lipid referred to herein as “C12-200” is disclosed by Love et al., Proc Natl Acad Sci USA. 2010 107:1864-1869 and Liu and Huang, Molecular Therapy. 2010 669-670; both of which are herein incorporated by reference in their entirety. Cationic lipid formulations can include particles comprising either 3 or 4 or more components in addition to polynucleotide, primary construct, or RNA (e.g. mRNA). As an example, formulations with certain cationic lipids include, but are not limited to, 98N12-5, and may contain 42% lipidoid, 48% cholesterol, and 10% PEG (C14 or greater alkyl chain length). As another example, formulations with certain lipidoids include, but are not limited to, C12-200 and may contain 50% cationic lipid, 10% disteroylphosphatidyl choline, 38.5% cholesterol, and 1.5% PEG-DMG.

In some embodiments, lipid nanoparticles are formulated as described in U.S. Pat. No. 10,709,779B2, which is incorporated in its entirety by reference herein. In some embodiments, the cationic lipid nanoparticle comprises a cationic lipid, a PEG-modified lipid, a sterol, and a non-cationic lipid. In some embodiments, the cationic lipid is selected from the group consisting of 98N12-5 (TETA5-LAP), DLin DMA, DLin-K-DMA (2,2-Dilinoleyl-4-dimethylaminomethyl-[1,3]-dioxolane), DLin-KC2-DMA, DLin-MC3-DMA, and C12-200. In some embodiments, the cationic lipid nanoparticle has a molar ratio of about 20-60% cationic lipid, about 5-25% non-cationic lipid, about 25-55% sterol, and about 0.5-15% PEG-modified lipid. In some embodiments, the cationic lipid nanoparticle comprises a molar ratio of about 50% cationic lipid, about 1.5% PEG-modified lipid, about 38.5% cholesterol, and about 10% non-cationic lipid. In some embodiments, the cationic lipid nanoparticle comprises a molar ratio of about 55% cationic lipid, about 2.5% PEG-modified lipid, about 32.5% cholesterol, and about 10% non-cationic lipid. In some embodiments, the cationic lipid is an ionizable cationic lipid, the non-cationic lipid is a neutral lipid, and the sterol is a cholesterol. In some embodiments, the cationic lipid nanoparticle has a molar ratio of 50:38.5:10:1.5 of cationic lipid:cholesterol: PEG2000-DMG:DSPC or DMG:DOPE. In some embodiments, lipid nanoparticles as described herein can comprise cholesterol, 1,2-dioleoyl-sn-glycero-3-phosphoethanolamine (DOPE), 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200), and DMG-PEG-2000 at molar ratios of 47.5:16:35:1.5.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### EXAMPLES

### Example 1—Gene Editing Outcomes at the DNA Level for Human GPR146 in Hep3B Cells

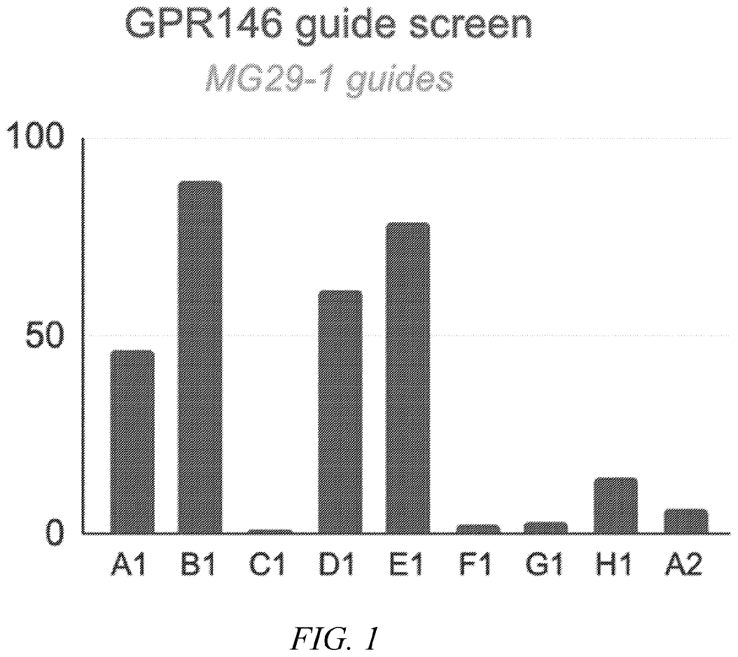

Nucleofection of MG29-1 RNPs (126 μmol protein/160 μmol guide) was performed into Hep3B cells (100,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 1).

### Example 2—Gene Editing Outcomes at the DNA Level for Mouse GPR146 in Hepa1-6 Cells

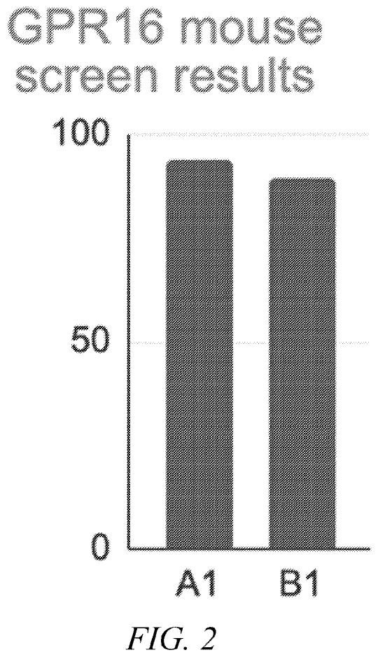

Nucleofection of MG29-1 RNPs (126 μmol protein/160 μmol guide) was performed into Hepa1-6 cells (100,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing. (FIG. 2).

### Example 3—Gene Editing Outcomes at the DNA Level for Human ANGPTL3 in Hep3B Cells

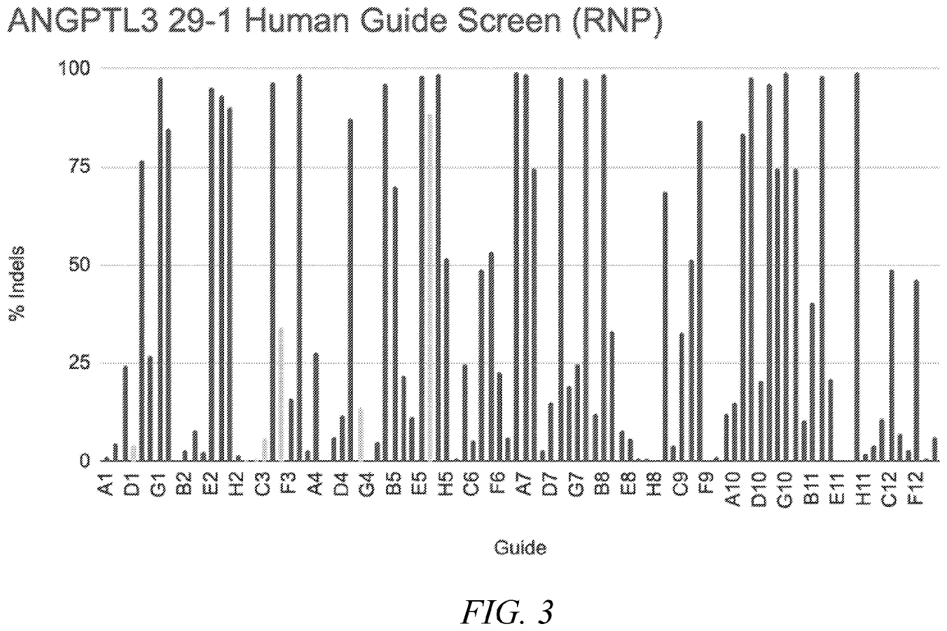

Nucleofection of MG29-1 RNPs (126 μmol protein/160 μmol guide) was performed into Hep3B cells (100,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 3).

### Example 4—Gene Editing Outcomes at the DNA Level for Human GPR145 in Primary Human Hepatocytes

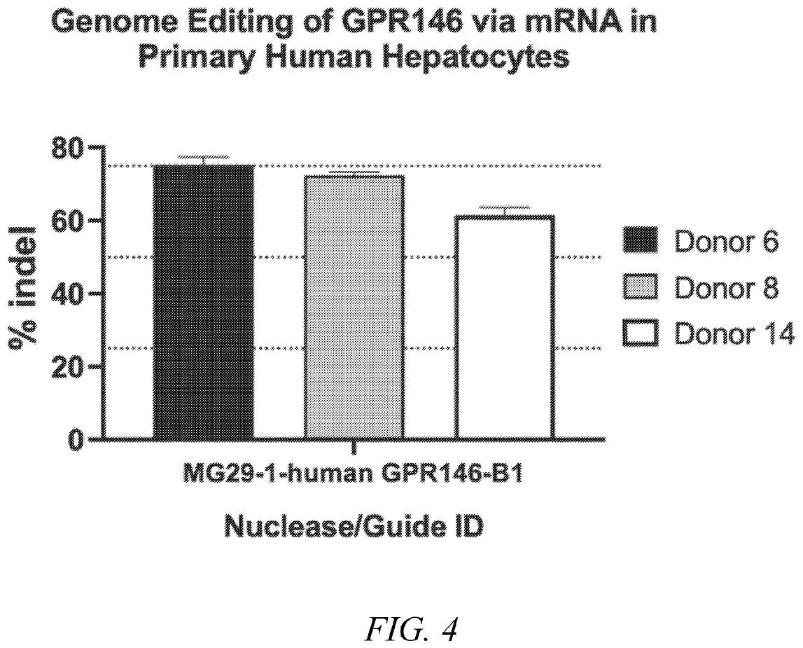

Lipofection with MessengerMax of MG29-1 mRNA and guide (1.25 μg mRNA, 1:20 nuclease: guide molar ratio) was performed in primary human hepatocytes (8×10⁵-1.0×10⁶ viable cells/guide depending on donor). Cells were harvested and genomic DNA prepared three days post-transfection. PCR primers appropriate for use in NGS-based DNA sequencing were used to amplify the individual target sequences for each guide RNA. The amplicons were sequenced on an Illumina MiSeq machine and analyzed with a proprietary Python script to measure gene editing (FIG. 4).

### Example 5—Gene Editing Outcomes at the DNA Level for Mouse GPR145 in Primary Mouse Hepatocytes

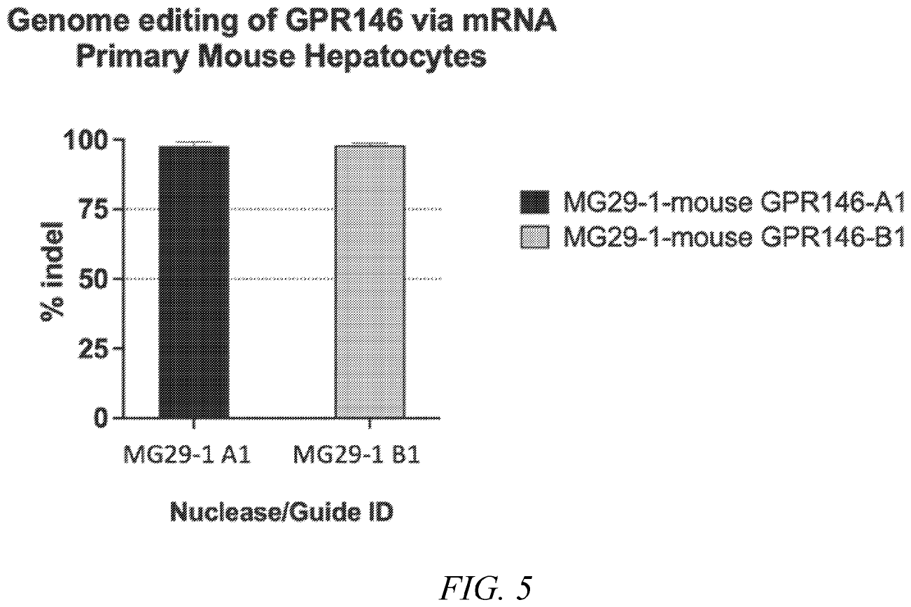

Lipofection with MessengerMax of MG29-1 mRNA and guide (0.42 μg mRNA, 1:20 nuclease: guide molar ratio) was performed in primary mouse hepatocytes (1×10⁵ viable cells/guide). Cells were harvested and genomic DNA prepared three days post-transfection. PCR primers appropriate for use in NGS-based DNA sequencing were used to amplify the individual target sequences for each guide RNA. The amplicons were sequenced on an Illumina MiSeq machine and analyzed with a proprietary Python script to measure gene editing (FIG. 5).

### Example 6—in Silico Identification of Novel Type V Nucleases in the MG29 and MG91 Families

Homology searches were performed to discover proteins predicted to be related to nuclease sequences in the MG29 family of large type V nucleases and the MG91 family of compact type V nucleases. Searches were performed using HMMER software (http://hmmer.org/). Large type V sequence hits were retained if the hmmsearch e-value was ≤10⁻⁵ and the amino acid sequence length was greater than or equal to 700 amino acids. Compact type V nuclease sequence hits were retained if: (1) the hmmsearch e-value was ≤10⁻⁵, (ii) the genes encoding the nuclease were within 1 Kb from a CRISPR array, and (iii) the amino acid sequence length ranged between 350 and 700 aa. MMSegs2 (https://github.com/soedinglab/MMseqs2) was used to separately cluster sequences at 100% amino acid identity, with coverage mode 1 and 80% coverage of the target sequence (parameters --cov-mode 1-c 0.8 --min-seq-id 1.0). Sequence representatives for each family were chosen to build a multiple sequence alignment using MAFFT (https://mafft.cbrc.jp/alignment/software/) with the Needleman-Wunsch algorithm for global alignment, and FastTree (https://doi.org/10.1371/journal.pone.0009490) was used to build a phylogenetic tree. Novel sequences clustering with previously discovered MG29 sequences were identified as additional members of the MG29 family. Additionally, careful examination of individual clades on the compact type V tree phylogenetic tree led to the identification of novel nuclease sequences in the MG91 family (SEQ ID NOs: 6274-6281).

### Example 7—MG91 Family sgRNA Prediction and Nuclease Activity Assays

De Novo Prediction of tracrRNA Sequences Encoded in Intergenic Regions

Compact type V MG91 nuclease proteins from a distinct clade were targeted for in silico characterization of genomic regions encoding CRISPR Cas systems. To identify intergenic regions potentially encoding tracrRNAs, individual protein clades (with confirmed catalytic residues) were chosen for visual inspection of contigs encoding the compact type V nuclease genes and a CRISPR array. Genomic regions devoid of coding sequence predictions between two genes, or between genes and CRISPR arrays, were manually annotated as intergenic regions. Intergenic regions upstream and downstream from a nuclease gene as well as a CRISPR array (e.g., at the same location relative to a nuclease and the corresponding CRISPR array) were consistently assigned labels across contigs encoding homologous nucleases. Nucleotide sequences of matching intergenic regions were aligned and inspected for conserved motifs across sequences. Similarly, nucleotide sequences from non-matching intergenic regions within clades were aligned and inspected. By comparison, intergenic regions with the highest degree of conservation among them were identified as potentially encoding tracrRNAs (e.g., SEQ ID NO: 6282).

Mapping Active tracrRNA Sequences to Contigs for Identification of tracrRNA Boundaries

To refine the boundaries of the non-coding intergenic region containing a potential tracrRNA for the MG91-2 nuclease, the sequence of an active tracrRNA from previous assays was mapped to the contig using Geneious 2022.2.2 (https://www.geneious.com). The aligned region was extracted and inferred to correspond to the actual sequence of the tracrRNA.

Covariance Model Prediction of Novel tracrRNA Sequences

Previously discovered active tracrRNA sequences in the MG91 family were used to generate covariance models to predict additional tracrRNAs. Covariance models were built from a multiple sequence alignment (MSA) of the active and predicted tracrRNA sequences. The secondary structure of the MSA was obtained with RNAalifold (Vienna Package), and the covariance models were built with Infernal packages (http://eddylab.org/infernal/). Contigs containing candidate nucleases (e.g., MG91-10, MG91-69, MG91-107, MG91-155, MG91-201, MG91-666, MG91-668, MG91-671, MG91-672 and MG91-673) were searched using the covariance models with the Infernal command ‘cmsearch’. TracrRNA candidates were tested in vitro, and in an iterative process, sequences from active candidates were used to improve the covariance models and search for additional tracrRNAs in the intergenic regions associated with other nuclease candidates.

Secondary Structure Prediction and sgRNA Design

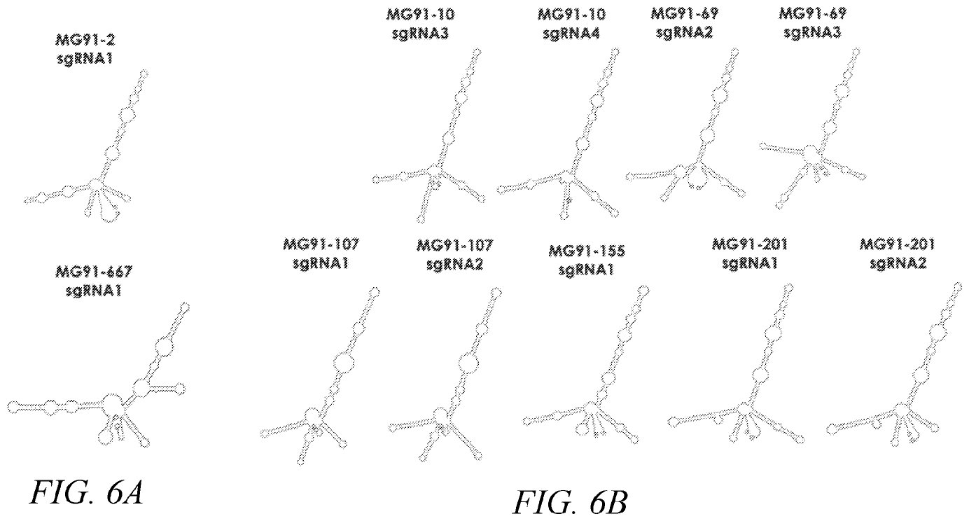

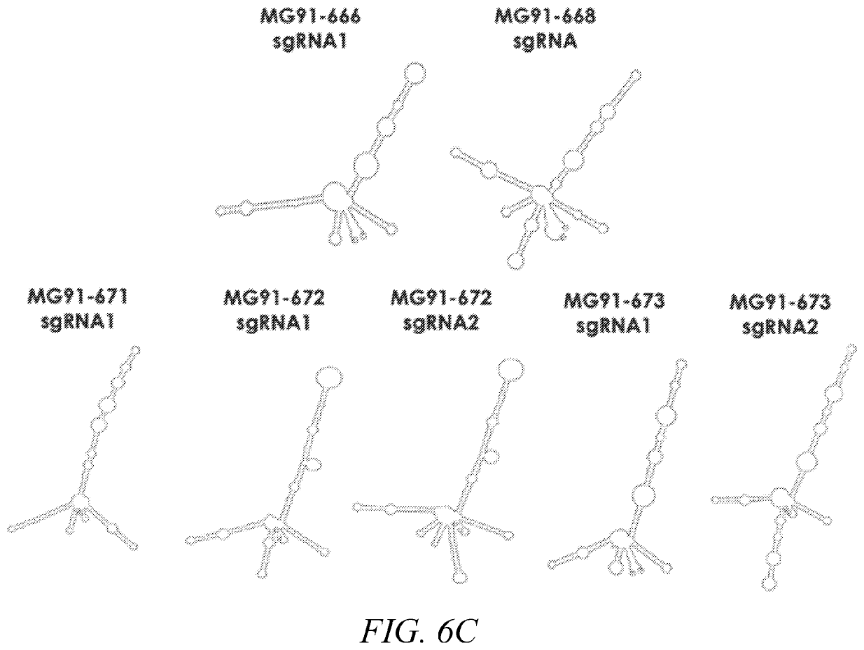

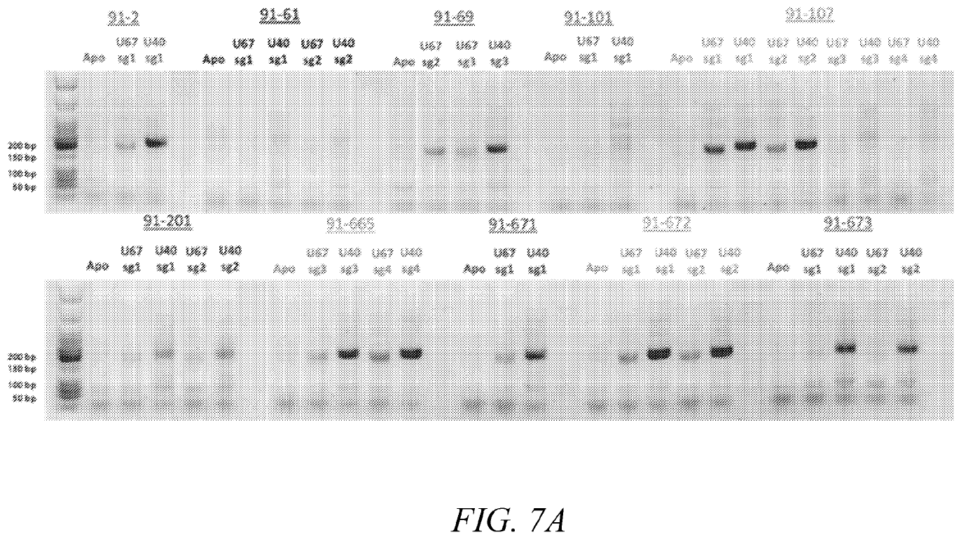

Intergenic regions (and extractions) potentially encoding tracrRNAs for MG91-2, MG91-667 (SEQ ID NO: 6275), and MG91-670 (SEQ ID NO: 6278) nucleases were folded with the corresponding repeat sequences using different energy models (Turner 2004 or Andronescu 2007) and parameters (for example, 20° C., 37° C., dangling ends) (FIG. 6A). Similarly, covariance model-predicted tracrRNAs for nucleases MG91-10, MG91-69, MG91-107, MG91-155, MG91-201, MG91-666 (SEQ ID NO: 6274), MG91-668 (SEQ ID NO: 6276), MG91-671 (SEQ ID NO: 6279), MG91-672 (SEQ ID NO: 6280), and MG91-673 (SEQ ID NO: 6281), and their associated CRISPR repeat sequence, were folded for sgRNA secondary structure prediction (FIGS. 6B and 6C). The stability of potential secondary RNA structures was visually inspected based on base pairs probabilities.

All folds with high base pair probabilities were modified to generate sgRNAs as follows: the 3′ end of the predicted tracrRNA sequence as well as the 5′ end of the repeat sequence were trimmed, and then connected with a GAAA tetraloop. At a later step, strings of four consecutive Us were replaced by single or paired mutations to prevent early termination of transcription and immunogenicity in mammalian cells, while preserving the secondary structure of the sgRNAs.

### In Vitro Cleavage Activity, PAM Sequence and Cut Site Determination

5 nM of nuclease amplified DNA templates and 25 nM sgRNA amplified DNA templates (including one of the spacer sequences listed in Table 2) were expressed at 37° C. for 3 hours with PURExpress® In Vitro Protein Synthesis Kit (New England Biolabs Inc.).

TABLE 2
Spacer sequences for tested guides

| Code | Sequence |
|---|---|
| U67 spacer | GTCGAGGCTTGCGACGTGGT |
| U40 spacer | TGGAGATATCTTGAACCTTG |

Plasmid library DNA cleavage reactions were carried out by mixing 5 nM of the target library representing all possible 8N PAMs, a 5-fold dilution of PURExpress expressions, 10 nM Tris-HCl, 10 nM MgCl₂ and, 100 mM NaCl at 37° C. for 2 hours. Reactions were stopped and cleaned with HighPrep™ PCR clean up beads (MAGBIO Genomics, Inc.) and eluted in Tris EDTA pH 8.0 buffer.

To obtain the PAM sequences and the target strand cleavage site, 3 nM of the cleavage product ends were blunted with 3.33 μM dNTPs, 1×T4 DNA ligase buffer, and 0.167 U/μL of Klenow Fragment (New England Biolabs Inc.) at 25° C. for 15 minutes. 1.5 nM of the cleavage products were ligated with 150 nM adapters, 1×T4 DNA ligase buffer (New England Biolabs Inc.), and 20 U/μL T4 DNA ligase (New England Biolabs Inc.) at room temperature for 20 minutes. The ligated products were amplified by PCR with NGS primers and sequenced by NGS.

To obtain the non-target strand cleavage site, 3 nM of the cleavage product ends were blunted with 0.167 U/μL of Mung Bean Nuclease and 1× Mung Bean Nuclease Buffer (New England Biolabs Inc.) at 30° C. for 30 minutes. The ligated products were amplified by PCR with NGS primers and sequenced by NGS.

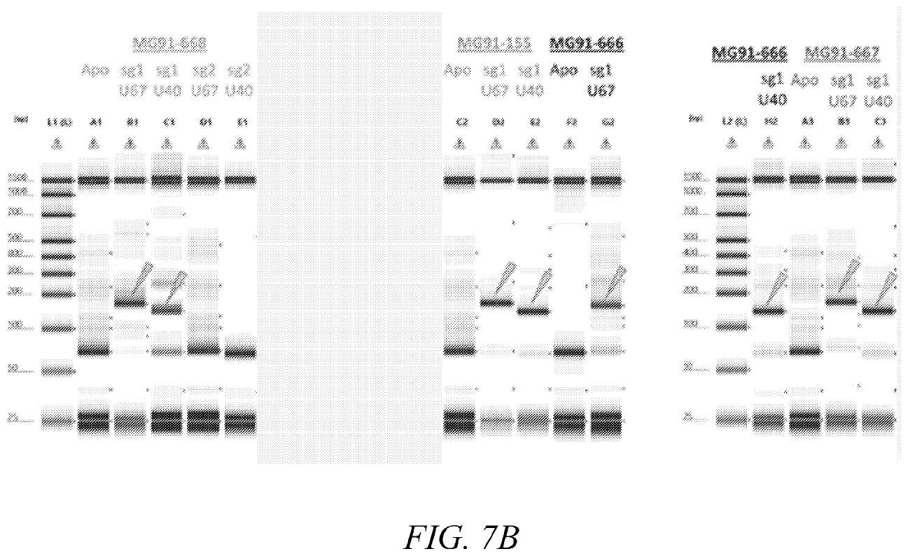

Active proteins that successfully cleaved the PAM library yielded a band around 188 or 205 bp in an agarose gel or an Agilent technologies 4200 TapeStation and D1000 ScreenTape System (FIG. 7).

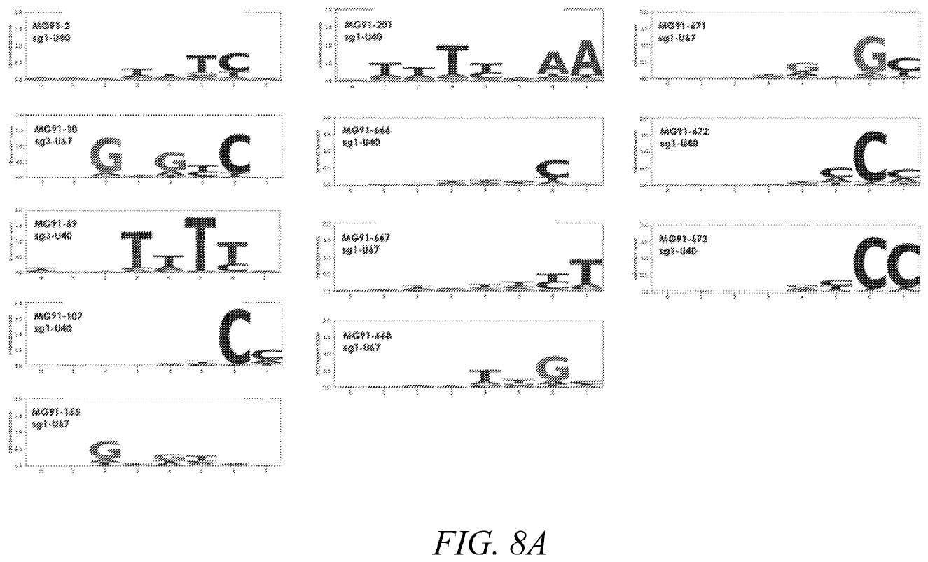

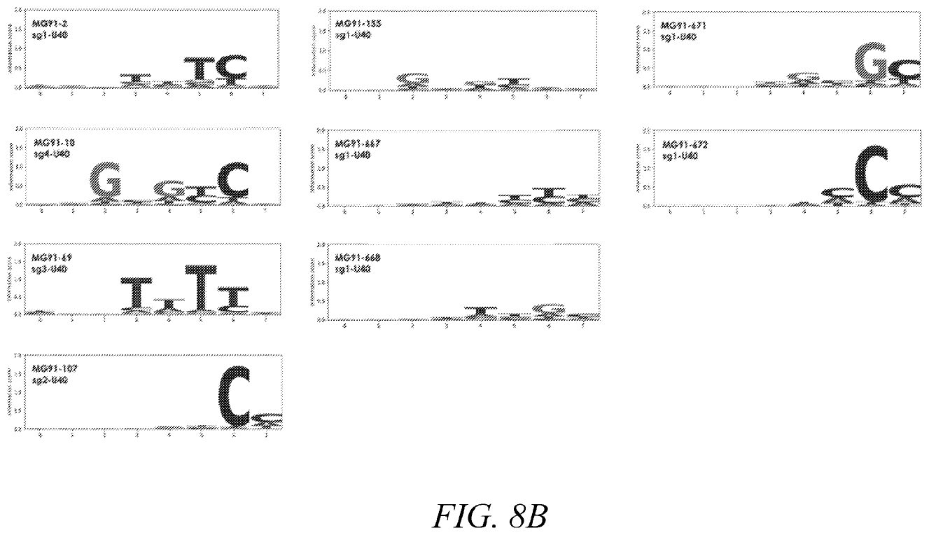

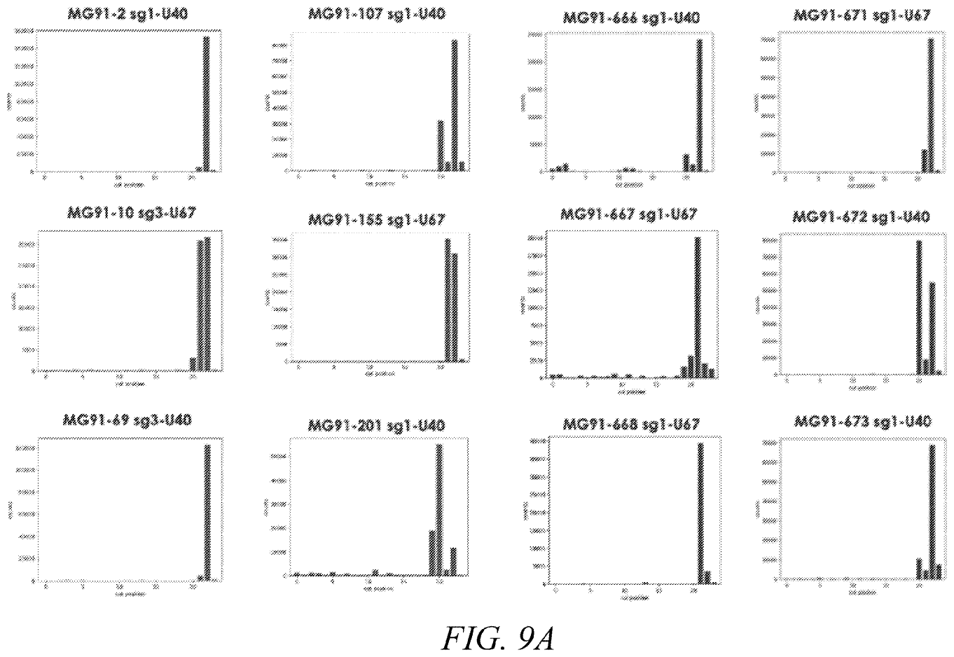

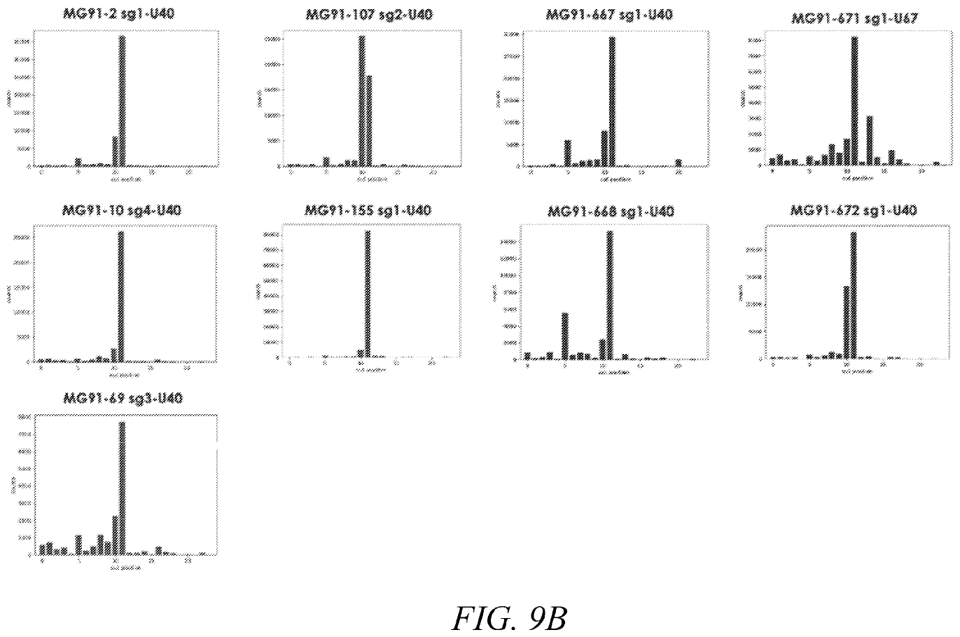

PAM sequence logos were made using Seqlogo maker, for both the target strand (FIG. 8A) and in most cases, the non-target strand (FIG. 8B) as well. Histograms of the cut sites obtained for the target strand (FIG. 9A) and the non-target strand (FIG. 9B) were made from the counts of reads at each nucleotide position. The preferred cut positions are shown in FIGS. 9A and 9B and Table 3.

TABLE 3
MG91 nucleases preferred cut site

| | | Target strand | Non-target strand |
|---|---|---|---|
| Nuclease | sgRNA | cutsite | cutsite |
| MG91-2 | MG91-2 sgRNA1 | 22 | 11 |
| MG91-10 | MG91-10 sgRNA2 | 22 | 11 |
| | MG91-10 sgRNA3 | | |
| MG91-69 | MG91-69 sgRNA3 | 22 | 11 |
| MG91-107 | MG91-107 sgRNA1 | 22 | 10 & 11 |
| | MG91-107 sgRNA2 | | |
| MG91-155 | MG91-155 sgRNA1 | 21-22 | 11 |
| MG91-201 | MG91-201 sgRNA1 | 20 | TBD |
| MG91-666 | MG91-666_sgRNA1 | 22 | TBD |
| MG91-667 | MG91-667_sgRNA1 | 21 | 11 |
| MG91-668 | MG91-668_sgRNA1 | 21 | 11 |
| MG91-671 | MG91-671_sgRNA1 | 22 | 11 |
| MG91-672 | MG91-672_sgRNA1 | 20 & 22 | 10 & 11 |
| MG91-673 | MG91-673_sgRNA1 | 22 | TBD |

### Example 8—sgRNA Structural Engineering (Prophetic)

Some of the designed guides for effector candidates are long, and this length may pose an obstacle to large-scale production. Furthermore, guide RNAs can be engineered to be smaller, more active, or both. Informed by predicted sgRNA structures, various truncations of guide RNAs are designed and synthesized. Guide quality is evaluated by in vitro RNP activity (cleavage of a linear DNA substrate), RNP stability, and in vivo editing in mammalian cells. Following the initial round of screening, combinatorial truncations are designed in sequential rounds to further improve guide design.

### Example 9—Protein Expression and Purification (Prophetic)

Isolating pure and functional proteins is essential for extensive in vitro analysis of biochemical properties and mechanistic studies. The expression and purification of MG91 candidates is optimized to obtain proteins of sufficient quantity and quality for such characterizations. All constructs are expressed in E. coli (NEBExpress Iq Competent E. coli, NEB C30371). Constructs are expressed in either the pMGB expression vector (MBP-fused), or the pMGBΔ expression vector (no fusion protein).

### Protein Expression Optimization

Protein expression protocols for all vectors are similar. Effector constructs are cloned into various expression vectors, with priority for expression without a fusion protein (pMGBΔ). In cases where expression or yield is insufficient, effectors are expressed with either an N-terminal MBP fusion or N-terminal SUMO fusion (Table 4).

TABLE 4
Sequence element glossary

| Element name | Element amino acid sequence |
|---|---|
| 6xHis | HHHHHH |
| (GS)ₙ | GS |
| (GGS)ₙ | GGS |
| (GGGGS)ₙ | GGGGS |
| PSP | LEVQFQGP |
| TEV | ENLYFQG |
| Nucleoplasmin | KRPAATKKAGQAKKKK |
| bipartite NLS | |
| SV40 NLS | PKKKRKV |
| SUMO sequence | TCGGACTCAGAAGTCAATCAAGAAGCTAAGCCA |
| | GAGGTCAAGCCAGAAGTCAAGCCTGAGACTCAC |
| | ATCAATTTAAAGGTGTCCGATGGATCTTCAGAGA |
| | TCTTCTTCAAGATCAAAAAGACCACTCCTTTAAG |
| | AAGGCTGATGGAAGCGTTCGCTAAAAGACAGGG |
| | TAAGGAAATGGACTCCTTAAGATTCTTGTACGAC |
| | GGTATTAGAATTCAAGCTGATCAGACCCCTGAAG |
| | ATTTGGACATGGAGGATAACGATATTATTGAGGC |
| | TCACAGAGAACAGATTGGTGGA |

One possible workflow is to remove the fusion protein with a targeted protease. Regardless of expression vector, cultures are grown at 37° C. in 2×YT media (1.6% tryptone, 1% yeast extract, 0.5% NaCl) or TB media (Tcknova T0690) with 100 μg/L Carbonicillin. At OD600≈0.8-1.2, cultures are induced with 0.5 mM IPTG (GoldBio 12481) and incubated at 18° C. overnight or 24° C. for 4-6 hrs, depending on construct. Cultures are then harvested by centrifugation at 6,000×g for 10 min, and pellets are resuspended in Nickel_A Buffer (50 mM Tris pH 7.5, 750 mM NaCl, 10 mM MgCl₂, 20 mM imidazole, 0.5 mM EDTA, 5% glycerol, 0.5 mM TCEP) with protease inhibitors (Pierce Protease Inhibitor Tablets, EDTA-free, ThermoFisher A32965) and stored at −80° C.

Protein Purification without a Fusion Protein

Proteins expressed in this vector have the following sequence architecture: 6×His-(GS) 2-PSP-nucleoplasmin bipartite NLS-(GGS) 1-(GS) 1-MG91-X-(GGS) 3-SV40 NLS (Table 4). Proteins expressed in this vector are denoted MG91-XA. Cell pellets are thawed and the volume supplemented to 120 mL with Cf=0.5% n-Octyl-β-D-glucoside detergent (P212121, CI-00234). Samples are sonicated in an ice-water bath at 75% amplitude for a total processing time of 3 min using a 15 s on/45 s off cycle. Lysates are clarified by centrifugation at 30,000×g for 25 min, and supernatants batch bound to 5 mL Ni-NTA resin (HisPur Ni-NTA Resin, ThermoFisher 88223) for ≥20 min. Samples are loaded onto a gravity column and washed with 30 CV Nickel_A Buffer, then eluted in 4 CV Nickel_B Buffer (Nickel_A Buffer+250 mM imidazole) before concentrating in a 50 kDa MWCO concentrator (Amicon Ultra-15, MilliporeSigma UFC9050). Samples are taken throughout the purification process and run on an SDS-PAGE protein gel (BioRad #4568126), which is imaged on a ChemiDoc in the stain-free channel following 5 min UV activation. Effectors are then loaded onto an S200i 10/300 GL column (Cytiva 28-9909-44) and run into SEC buffer (20 mM Tris·HCl pH 7.5, 250 mM NaCl, 10 mM MgCl₂, 0.5 mM TCEP, 0.5 mM EDTA, 10% glycerol). Peak fractions are pooled and concentrated in a 50 kDa MWCO concentrator.

Protein Purification with a Fusion Protein

Proteins expressed in this vector have one of the following sequence architectures: 6×His-(GS) 1-MBP-(GS) 1-TEV-nucleoplasmin bipartite NLS-(GGGGS) 3-(GS) 1-MG91-X-(GGS) 3-SV40 NLS, or 6×His-(GS) 1-SUMO-nucleoplasmin bipartite NLS-GGSGS-MG91-X-(GGS) 3-SV40 NLS (Table 4). Constructs are purified identically to non-fused proteins through lysis, clarification, affinity purification, elution in Nickel_B, and concentration in a 50 kDa MWCO concentrator. If the fusion protein is to be removed from the MBP-fused constructs, TEV protease (GenScript Z03030) is added to each sample (Cf=1 UI/μL) and incubated at 4° C. overnight, gently rotating end-over-end. Samples are then centrifuged (21,000×g, 4° C., 10 min) to pellet aggregates, and the supernatant is then batch-bound to 3 mL amylose resin (NEB E8021L) for 30 min at 4° C., then loaded onto a gravity column. The flow-through is collected and concentrated in a 50 kDa MWCO concentrator. Again, samples are centrifuged (21,000×g, 4° C., 10 min) to pellet aggregates before loading on an S200i 10/300 GL column and run into SEC buffer (20 mM Tris HCl pH 7.5, 250 mM NaCl, 10 mM MgCl₂, 0.5 mM TCEP, 0.5 mM EDTA, 10% glycerol). Peak fractions are pooled and concentrated in a 50 kDa MWCO concentrator. Samples are taken throughout the purification process and run on an SDS-PAGE protein gel (BioRad #4568126), which is imaged on a ChemiDoc in the stain-free channel following 5 min UV activation.

### Example 10—In Vitro Cleavage Efficiency with Purified Protein (Prophetic)

The active fraction of protein aliquots is determined in a linear DNA substrate cleavage assay. Effector proteins are preincubated with a 2-fold molar excess of sgRNA for 20 min at room temperature to form the ribonucleoprotein complex (RNP). Reactions are set up using 25 nM DNA substrate and a titration of RNP from 0.25× to 10× molar excess over substrate. The reaction buffer contains 10 mM Tris pH 7.5, 10 mM MgCl₂, and 100 mM NaCl. The DNA substrate is 522 bp long. Successful cleavage results in fragments of 172 and 350 bp. The reaction is incubated at 37° C. for 60 min, then incubated at 75° C. for 10 min. RNase (NEB T3018) is added to each reaction (Cf=0.33 μg/L), and samples are incubated at 37° C. for 10 min. Proteinase K (NEB P8107) is added to each reaction (Cf=60 units/mL), and samples are incubated at 55° C. for 15 min. The entirety of each reaction is then run on a 1.5% agarose gel with GelGreen dye (Biotium, #41005) and imaged on a ChemiDoc in the GelGreen channel. Percent cleaved substrate is calculated for each lane through densitometry analysis using BioRad's Image Lab software (Version 6.1.0 build 7). Active fraction is determined by the slope of the linear range of cleavage. Using this assay, effector activity is measured using sgRNAs with various spacer lengths (16 nt-26 nt) to determine the optimal spacer length for each effector.

### Example 11—Fluorescence-Based Measurement of Nuclease Activity (Prophetic)

### Novel Cell Line Engineering

Current assays used to measure in vivo (i.e., in mammalian cell lines) nuclease activity require extensive data analysis and turnaround times of up to a week. To expedite evaluation of in vivo nuclease activity, an immortalized mammalian cell line is engineered to provide immediate data on editing of genomic DNA. K562 mammalian cells, grown in IMDM (Gibco #12440053) and 10% FBS (Corning™ Regular Fetal Bovine Serum, MT35011CV), are used for this assay. K562 mammalian cells are transfected with 12 μmol Cas9 protein (IDT #1081058), 60 μmol sgRNA (Mali et al. Science 2013, 339 (6121), 823-826.), and 1200 ng plasmid (pUC backbone) containing an expression sequence for an mMBP-(GGS) 3-eGFP protein, as well as a gene for resistance to hygromycin to use as a selection marker. Genomic integration of this construct results in constitutive expression under the synthetic MND promoter. Cells are left to grow in the presence of hygromycin for 6 days, passaging every 3 days. Monogenic cell lines are isolated from single cells by sorting individual GFP-expressing cells into a 96-well plate using a Sony MA900 Cell Sorter.

### Fluorescence-Based In-Vivo Nuclease Activity Screen

Appropriate sgRNAs are designed to direct nuclease cleavage along the mMBP and eGFP genes, such that indel formation produces a frameshift mutation resulting in loss of fluorescence. RNP complexes are formed by combining 100 μmol protein and 120-200 μmol sgRNA and incubating at room temperature for ≥20 min in a final volume of 5 μL. K562 cells are washed in 1×PBS and resuspended in Nucleofector Solution (SF Cell Line 96-well Nucleofector™ Solution) with approximately 200,000 cells per well. Cells and RNP are combined in a Lonza 96-well nucleofection plate (SF Cell Line 96-well Nucleofector™ Kit, V4SC-2096) in a final volume of 25 μL, nucleofected (K562 cells, FF-120), and recovered in IMDM+10% FBS media+hygromycin. Cells are left to recover for 2-3 days at 37° C. To analyze, cells are washed twice with 1×PBS, then stained with 1×PBS+LIVE/DEAD Fixable Near-IR Dead Cell Stain Kit dye (ThermoFisher L10119) for 20 min at room temperature. Cells are washed once more with 1×PBS before being resuspended in 1×PBS and loaded into an Attune NxT, Acoustic Focusing Flow Cytometer (model AFC2) for fluorescence analysis. Positive unedited controls (nucleofected without RNP) and negative controls (non-fluorescent K562 cells) are used to establish positive and negative fluorescence gates, and cell populations are analyzed for loss-of-fluorescence in the GFP channel to evaluate in vivo nuclease activity.

### Example 12—Gene Editing in Human Cells (Prophetic)

K562 cells purchased from ATCC are cultured according to ATCC protocols. sgRNAs targeting the TRAC or AAVS1 loci are designed based on a set of MG91-recognized PAMs and plasmid-encoded guides. For gene editing experiments, 500 ng of in vitro-synthesized nuclease mRNA and a titration of the indicated sgRNA encoded in a plasmid with a U6 promoter or engineered chemically-synthesized sgRNA are co-nucleofected in 1.5×10⁵ cells using the Lonza 4D Nucleofector (program FF-120). Cells are harvested 72 hours post-electroporation for genomic DNA extraction using QuickExtract (Lucigen #09050) and processed for amplicon next-generation sequencing on an Illumina Miseq. The resulting data are analyzed with an indel calculator script.

TABLE 5
Listing of PAMs referred to herein not included in the sequence listing

| SEQ ID NO: | Description | Type | Organism | Sequence or Comment |
|---|---|---|---|---|
| 3863 | MG19-2 PAM | nucleotide | artificial sequence | TA (Sanger only) |
| 3864 | MG19-3 PAM | nucleotide | artificial sequence | TA (Sanger only) |
| 3865 | MG19-4 PAM | nucleotide | artificial sequence | TR |
| 3866 | MG19-5 PAM | nucleotide | artificial sequence | TTR (Sanger only) |
| 3867 | MG20-1 PAM | nucleotide | artificial sequence | TTA |
| 3868 | MG28-1 PAM (5′) from NGS | nucleotide | artificial sequence | TTTN |
| 3870 | MG29-1 PAM (5′) | nucleotide | artificial sequence | KTTG |
| 3871 | MG29-1 PAM (5′) from NGS | nucleotide | artificial sequence | YYN |
| 3872 | MG29-5 PAM | nucleotide | artificial sequence | YYYN |
| 3873 | MG30-1 PAM (5′) | nucleotide | artificial sequence | TTTn |
| 3874 | MG31-1 PAM | nucleotide | artificial sequence | TTTR |
| 3875 | MG31-1 PAM (5′) from NGS | nucleotide | artificial sequence | YTTN |
| 3876 | MG31-1, MG32-1 PAM (5′) | nucleotide | artificial sequence | YTTm |
| 3877 | MG32-1 PAM (5′) from NGS | nucleotide | artificial sequence | TTTN |
| 3879 | MG57-1 PAM | nucleotide | artificial sequence | YN |
| 3880 | MG57-2 PAM | nucleotide | artificial sequence | YYNW |
| 3881 | MG59-1 PAM | nucleotide | artificial sequence | YYN |
| 3882 | MG59-2 PAM | nucleotide | artificial sequence | YTTV |
| 3883 | MG77-1 PAM | nucleotide | artificial sequence | TTn |
| 3884 | MG77-2 PAM | nucleotide | artificial sequence | TTn |
| 3885 | MG78-1 PAM | nucleotide | artificial sequence | YYN |
| 3886 | MG79-1 PAM | nucleotide | artificial sequence | TTR |
| 3887 | MG79-2 PAM | nucleotide | artificial sequence | TTR (Sanger only) |
| 3888 | MG79-3 PAM | nucleotide | artificial sequence | TTTn (Sanger only) |
| 3889 | MG79-4 PAM | nucleotide | artificial sequence | TTn (Sanger only) |
| 4012 | mAlb29-1-1 PAM | nucleotide | artificial sequence | TTTA |
| 4013 | mAlb29-1-2 PAM | nucleotide | artificial sequence | GTTC |
| 4014 | mAlb29-1-3 PAM | nucleotide | artificial sequence | GTTG |
| 4015 | mAlb29-1-4 PAM | nucleotide | artificial sequence | TTTA |
| 4016 | mAlb29-1-5 PAM | nucleotide | artificial sequence | TTTT |
| 4017 | mAlb29-1-7 PAM | nucleotide | artificial sequence | TTTT |
| 4018 | mAlb29-1-8 PAM | nucleotide | artificial sequence | TTTC |
| 4019 | mAlb29-1-9 PAM | nucleotide | artificial sequence | GTTG |
| 4020 | mAlb29-1-10 PAM | nucleotide | artificial sequence | TTTT |
| 4021 | mAlb29-1-11 PAM | nucleotide | artificial sequence | TTTG |
| 4022 | mAlb29-1-12 PAM | nucleotide | artificial sequence | TTTG |
| 4023 | mAlb29-1-13 PAM | nucleotide | artificial sequence | TTTT |
| 4024 | mAlb29-1-14 PAM | nucleotide | artificial sequence | TTTT |
| 4025 | mAlb29-1-15 PAM | nucleotide | artificial sequence | TTTT |
| 4026 | mAlb29-1-16 PAM | nucleotide | artificial sequence | TTTC |
| 4027 | mAlb29-1-17 PAM | nucleotide | artificial sequence | TTTG |
| 4028 | mAlb29-1-18 PAM | nucleotide | artificial sequence | TTTA |
| 4029 | mAlb29-1-19 PAM | nucleotide | artificial sequence | GTTC |
| 4030 | mAlb29-1-20 PAM | nucleotide | artificial sequence | TTTA |
| 4056 | hAlb g63 PAM | nucleotide | artificial sequence | TTTA |
| 4057 | hAlb g59 PAM | nucleotide | artificial sequence | TTTG |
| 4058 | hAlb g58 PAM | nucleotide | artificial sequence | TTTT |
| 4059 | hAlb_g56 PAM | nucleotide | artificial sequence | TTTA |
| 4060 | hAlb g72 PAM | nucleotide | artificial sequence | TTTC |
| 4061 | hAlb_g70 PAM | nucleotide | artificial sequence | TTTT |
| 4062 | hAlb_g74 PAM | nucleotide | artificial sequence | TTTA |
| 4063 | hAlb g83 PAM | nucleotide | artificial sequence | TTTA |
| 4064 | hAlb_g85 PAM | nucleotide | artificial sequence | TTTC |
| 4065 | hAlb g89 PAM | nucleotide | artificial sequence | TTTT |
| 4066 | hAlb g88 PAM | nucleotide | artificial sequence | TTTT |
| 4067 | hAlb g77 PAM | nucleotide | artificial sequence | TTTA |
| 4068 | hAlb g69 PAM | nucleotide | artificial sequence | TTTT |
| 4069 | hAlb g66 PAM | nucleotide | artificial sequence | TTTG |
| 4070 | hAlb g75 PAM | nucleotide | artificial sequence | TTTT |
| 4071 | hAlb g79 PAM | nucleotide | artificial sequence | TTTC |
| 4072 | hAlb g82 PAM | nucleotide | artificial sequence | TTTA |
| 4073 | hAlb g80 PAM | nucleotide | artificial sequence | TTTG |
| 4074 | hAlb g84 PAM | nucleotide | artificial sequence | TTTG |
| 4075 | hAlb g81 PAM | nucleotide | artificial sequence | TTTT |
| 4076 | hAlb_g90 PAM | nucleotide | artificial sequence | TTTA |
| 4077 | hAlb g87 PAM | nucleotide | artificial sequence | TTTG |
| 4078 | hAlb g86 PAM | nucleotide | artificial sequence | TTTT |
| 4126 | mH29-1 PAM | nucleotide | artificial sequence | TTTG |
| 4127 | mH29-2 PAM | nucleotide | artificial sequence | TTTT |
| 4128 | mH29-3 PAM | nucleotide | artificial sequence | TTTT |
| 4129 | mH29-4 PAM | nucleotide | artificial sequence | GTTG |
| 4130 | mH29-5 PAM | nucleotide | artificial sequence | GTTT |
| 4131 | mH29-6 PAM | nucleotide | artificial sequence | GTTG |
| 4132 | mH29-7 PAM | nucleotide | artificial sequence | GTTG |
| 4133 | mH29-8 PAM | nucleotide | artificial sequence | GTTC |
| 4134 | mH29-9 PAM | nucleotide | artificial sequence | GTTC |
| 4135 | mH29-10 PAM | nucleotide | artificial sequence | GTTG |
| 4136 | mH29-11 PAM | nucleotide | artificial sequence | GTTC |
| 4137 | mH29-12 PAM | nucleotide | artificial sequence | GTTG |
| 4138 | mH29-13 PAM | nucleotide | artificial sequence | GTTG |
| 4139 | mH29-14 PAM | nucleotide | artificial sequence | TTTT |
| 4140 | mH29-15 PAM | nucleotide | artificial sequence | TTTG |
| 4141 | mH29-16 PAM | nucleotide | artificial sequence | TTTC |
| 4142 | mH29-17PAM | nucleotide | artificial sequence | TTTG |
| 4143 | mH29-18 PAM | nucleotide | artificial sequence | TTTA |
| 4144 | mH29-19 PAM | nucleotide | artificial sequence | GTTG |
| 4145 | mH29-20 PAM | nucleotide | artificial sequence | GTTC |
| 4146 | mH29-21 PAM | nucleotide | artificial sequence | GTTA |
| 4147 | mH29-22 PAM | nucleotide | artificial sequence | GTTG |
| 4148 | mH29-23 PAM | nucleotide | artificial sequence | GTTG |
| 4149 | mH29-24 PAM | nucleotide | artificial sequence | TTTA |
| 4150 | mH29-25 PAM | nucleotide | artificial sequence | TTTA |
| 4151 | IH29-26 PAM | nucleotide | artificial sequence | TTTC |
| 4152 | mH29-27 PAM | nucleotide | artificial sequence | TTTG |
| 4153 | mH29-28 PAM | nucleotide | artificial sequence | TTTG |
| 4154 | mH29-29 PAM | nucleotide | artificial sequence | TTTC |
| 4155 | mH29-30 PAM | nucleotide | artificial sequence | TTTT |
| 4156 | mH29-31 PAM | nucleotide | artificial sequence | GTTA |
| 4157 | mH29-32 PAM | nucleotide | artificial sequence | GTTT |
| 4158 | mH29-33 PAM | nucleotide | artificial sequence | GTTT |
| 4159 | mH29-34 PAM | nucleotide | artificial sequence | GTTG |
| 4160 | mH29-35 PAM | nucleotide | artificial sequence | GTTG |
| 4161 | mH29-36 PAM | nucleotide | artificial sequence | TTTA |
| 4162 | mH29-37 PAM | nucleotide | artificial sequence | TTTC |
| 4163 | mH29-38 PAM | nucleotide | artificial sequence | TTTC |
| 4164 | mH29-39 PAM | nucleotide | artificial sequence | GTTA |
| 4165 | mH29-40 PAM | nucleotide | artificial sequence | TTTT |
| 4166 | mH29-41 PAM | nucleotide | artificial sequence | TTTG |
| 4167 | mH29-42 PAM | nucleotide | artificial sequence | TTTC |
| 4168 | mH29-43 PAM | nucleotide | artificial sequence | TTTG |
| 4169 | mH29-44 PAM | nucleotide | artificial sequence | TTTT |
| 4170 | mH29-45 PAM | nucleotide | artificial sequence | TTTG |
| 4226 | hH29-1 PAM | nucleotide | artificial sequence | TTTA |
| 4227 | hH29-2 PAM | nucleotide | artificial sequence | TTTG |
| 4228 | hH29-3 PAM | nucleotide | artificial sequence | TTTG |
| 4229 | hH29-4 PAM | nucleotide | artificial sequence | TTTG |
| 4230 | hH29-5 PAM | nucleotide | artificial sequence | TTTC |
| 4231 | hH29-6 PAM | nucleotide | artificial sequence | TTTC |
| 4232 | hH29-7 PAM | nucleotide | artificial sequence | TTTT |
| 4233 | hH29-8 PAM | nucleotide | artificial sequence | TTTG |
| 4234 | hH29-9 PAM | nucleotide | artificial sequence | TTTA |
| 4235 | hH29-10 PAM | nucleotide | artificial sequence | TTTT |
| 4236 | hH29-11 PAM | nucleotide | artificial sequence | TTTT |
| 4237 | hH29-12 PAM | nucleotide | artificial sequence | TTTA |
| 4238 | hH29-13 PAM | nucleotide | artificial sequence | TTTT |
| 4239 | hH29-14 PAM | nucleotide | artificial sequence | TTTA |
| 4240 | hH29-15 PAM | nucleotide | artificial sequence | TTTT |
| 4241 | hH29-16 PAM | nucleotide | artificial sequence | TTTT |
| 4242 | hH29-17 PAM | nucleotide | artificial sequence | TTTT |
| 4243 | hH29-18 PAM | nucleotide | artificial sequence | TTTA |
| 4244 | hH29-19 PAM | nucleotide | artificial sequence | TTTC |
| 4245 | hH29-20 PAM | nucleotide | artificial sequence | TTTT |
| 4246 | hH29-21 PAM | nucleotide | artificial sequence | TTTA |
| 4247 | hH29-22 PAM | nucleotide | artificial sequence | TTTC |
| 4248 | hH29-23 PAM | nucleotide | artificial sequence | TTTC |
| 4249 | hH29-24 PAM | nucleotide | artificial sequence | TTTG |
| 4250 | hH29-25 PAM | nucleotide | artificial sequence | TTTG |
| 4251 | hH29-26 PAM | nucleotide | artificial sequence | TTTT |
| 4252 | hH29-27 PAM | nucleotide | artificial sequence | TTTA |
| 4253 | hH29-28 PAM | nucleotide | artificial sequence | TTTC |
| 4254 | hH29-29 PAM | nucleotide | artificial sequence | TTTT |
| 4255 | hH29-30 PAM | nucleotide | artificial sequence | TTTA |
| 4256 | hH29-31 PAM | nucleotide | artificial sequence | TTTC |
| 4257 | hH29-32 PAM | nucleotide | artificial sequence | TTTT |
| 4258 | hH29-33 PAM | nucleotide | artificial sequence | TTTT |
| 4259 | hH29-34 PAM | nucleotide | artificial sequence | TTTG |
| 4260 | hH29-35 PAM | nucleotide | artificial sequence | TTTA |
| 4261 | hH29-36 PAM | nucleotide | artificial sequence | TTTT |
| 4262 | hH29-37 PAM | nucleotide | artificial sequence | TTTC |
| 4263 | hH29-38 PAM | nucleotide | artificial sequence | TTTA |
| 4264 | hH29-39 PAM | nucleotide | artificial sequence | TTTT |
| 4265 | hH29-40 PAM | nucleotide | artificial sequence | TTTG |
| 4266 | hH29-41 PAM | nucleotide | artificial sequence | TTTG |
| 4267 | hH29-42 PAM | nucleotide | artificial sequence | TTTG |
| 6037 | MG91-15 PAM (5′) | nucleotide | Unknown | TtTYn |
| 6038 | MG91-32 PAM (5′) | nucleotide | Unknown | GnYYn |
| 6039 | MG91-87 PAM (5′) | nucleotide | Unknown | wCCC |

TABLE 6
Listing of additional protein and nucleic acid sequences referred to herein not included in the sequence listing

| | SEQ | | | | |
|---|---|---|---|---|---|
| Cat. | ID: | Description | Type | Organism | Sequence |
| MG29-1 | 6060 | MG29-1-human | Nucleotide | N.A. | /AltR1/rUrArArUrUrUrCrUrArCrUrGrUrUrGrUrArGrArUrUrCrArArCrArUrGrGrCrArG |
| sgRNA | | GPR146-A1 | | | rUrGrGrCrArGrGrCrCrU/AltR2/ |
| targeting | | | | | |
| human | | | | | |
| GPR146 | | | | | |
| MG29-1 | 6061 | MG29-1-human | Nucleotide | N.A. | /AltR1/rUrArArUrUrUrCrUrArCrUrGrUrUrGrUrArGrArUrGrGrCrUrCrUrGrGrArCrGrC |
| sgRNA | | GPR146-B1 | | | rCrArCrArCrUrArUrCrU/AltR2/ |
| targeting | | | | | |
| human | | | | | |
| GPR146 | | | | | |
| MG29-1 | 6062 | MG29-1-human | Nucleotide | N.A. | /AltR1/rUrArArUrUrUrCrUrArCrUrGrUrUrGrUrArGrArUrUrGrArArGrGrArUrUrUrCrU |
| sgRNA | | GPR146-C1 | | | rCrCrArArArCrUrCrCrU/AltR2/ |
| targeting | | | | | |
| human | | | | | |
| GPR146 | | | | | |
| MG29-1 | 6063 | MG29-1-human | Nucleotide | N.A. | /AltR1/rUrArArUrUrUrCrUrArCrUrGrUrUrGrUrArGrArUrUrCrCrArArArCrUrCrCrUrG |
| sgRNA | | GPR146-D1 | | | rGrCrCrUrUrCrUrCrCrA/AltR2/ |
| targeting | | | | | |
| human | | | | | |
| GPR146 | | | | | |
| MG29-1 | 6064 | MG29-1-human | Nucleotide | N.A. | /AltR1/rUrArArUrUrUrCrUrArCrUrGrUrUrGrUrArGrArUrUrGrArCrArCrCrArCrUrUrC |
| sgRNA | | GPR146-E1 | | | rUrCrUrArCrCrGrCrUrA/AltR2/ |
| targeting | | | | | |
| human | | | | | |
| GPR146 | | | | | |
| MG29-1 | 6065 | MG29-1-human | Nucleotide | N.A. | /AltR1/rUrArArUrUrUrCrUrArCrUrGrUrUrGrUrArGrArUrUrCrArUrCrArGrCrCrGrUrU |
| sgRNA | | GPR146-F1 | | | rGrGrArGrCrUrUrGrCrU/AltR2/ |
| targeting | | | | | |
| human | | | | | |
| GPR146 | | | | | |
| MG29-1 | 6066 | MG29-1-human | Nucleotide | N.A. | /AltR1/rUrArArUrUrUrCrUrArCrUrGrUrUrGrUrArGrArUrCrArUrCrArGrCrCrGrUrUrG |
| sgRNA | | GPR146-G1 | | | rGrArGrCrUrUrGrCrUrG/AltR2/ |
| targeting | | | | | |
| human | | | | | |
| GPR146 | | | | | |
| MG29-1 | 6067 | MG29-1-human | Nucleotide | N.A. | /AltR1/rUrArArUrUrUrCrUrArCrUrGrUrUrGrUrArGrArUrArUrCrArGrCrCrGrUrUrGrG |
| sgRNA | | GPR146-H1 | | | rArGrCrUrUrGrCrUrGrG/AltR2/ |
| targeting | | | | | |
| human | | | | | |
| GPR146 | | | | | |
| MG29-1 | 6068 | MG29-1-human | Nucleotide | N.A. | /AltR1/rUrArArUrUrUrCrUrArCrUrGrUrUrGrUrArGrArUrGrArGrArArArUrCrCrUrUrC |
| sgRNA | | GPR146-A2 | | | rArCrArArArGrUrGrCrA/AltR2/ |
| targeting | | | | | |
| human | | | | | |
| GPR146 | | | | | |
| DNA | 6069 | MG29-1-human | Nucleotide | N.A. | TCAACATGGCAGTGGCAGGCCT |
| sequence | | GPR146-A1 | | | |
| of human | | | | | |
| GPR146 | | | | | |
| target site | | | | | |
| DNA | 6070 | MG29-1-human | Nucleotide | N.A. | GGCTCTGGACGCCACACTATCT |
| sequence | | GPR146-B1 | | | |
| of human | | | | | |
| GPR146 | | | | | |
| target site | | | | | |
| DNA | 6071 | MG29-1-human | Nucleotide | N.A. | TGAAGGATTTCTCCAAACTCCT |
| sequence | | GPR146-C1 | | | |
| of human | | | | | |
| GPR146 | | | | | |
| target site | | | | | |
| DNA | 6072 | MG29-1-human | Nucleotide | N.A. | TCCAAACTCCTGGCCTTCTCCA |
| sequence | | GPR146-D1 | | | |
| of human | | | | | |
| GPR146 | | | | | |
| target site | | | | | |
| DNA | 6073 | MG29-1-human | Nucleotide | N.A. | TGACACCACTTCTCTACCGCTA |
| sequence | | GPR146-E1 | | | |
| of human | | | | | |
| GPR146 | | | | | |
| target site | | | | | |
| DNA | 6074 | MG29-1-human | Nucleotide | N.A. | TCATCAGCCGTTGGAGCTTGCT |
| sequence | | GPR146-F1 | | | |
| of human | | | | | |
| GPR146 | | | | | |
| target site | | | | | |
| DNA | 6075 | MG29-1-human | Nucleotide | N.A | CATCAGCCGTTGGAGCTTGCTG |
| sequence | | GPR146-G1 | | | |
| of human | | | | | |
| GPR146 | | | | | |
| target site | | | | | |
| DNA | 6076 | MG29-1-human | Nucleotide | N.A. | ATCAGCCGTTGGAGCTTGCTGG |
| sequence | | GPR146-H1 | | | |
| of human | | | | | |
| GPR146 | | | | | |
| target site | | | | | |
| DNA | 6077 | MG29-1-human | Nucleotide | N.A. | GAGAAATCCTTCACAAAGTGCA |
| sequence | | GPR146-A2 | | | |
| of human | | | | | |
| GPR146 | | | | | |
| target site | | | | | |
| MG29-1 | 6078 | MG29-1-mouse | Nucleotide | N.A. | /AltR1/rUrArArUrUrUrCrUrArCrUrGrUrUrGrUrArGrArUrGrCrCrUrCrUrGrGrArCrArC |
| sgRNA | | GPR146-A1 | | | rCrUrUrArCrUrArCrUrU/AltR2/ |
| targeting | | | | | |
| mouse | | | | | |
| GPR146 | | | | | |
| MG29-1 | 6079 | MG29-1-mouse | Nucleotide | N.A. | /AltR1/rUrArArUrUrUrCrUrArCrUrGrUrUrGrUrArGrArUrUrUrGrArUrGrUrArArCrGrG |
| sgRNA | | GPR146-B1 | | | rUrArGrArGrCrArGrUrG/AltR2/ |
| targeting | | | | | |
| mouse | | | | | |
| GPR146 | | | | | |
| DNA | 6080 | MG29-1-mouse | Nucleotide | N.A. | GCCTCTGGACACCTTACTACTT |
| sequence | | GPR146-A1 | | | |
| of mouse | | | | | |
| GPR146 | | | | | |
| target site | | | | | |
| DNA | 6081 | MG29-1-mouse | Nucleotide | N.A. | TTGATGTAACGGTAGAGCAGTG |
| sequence | | GPR146-B1 | | | |
| of mouse | | | | | |
| GPR146 | | | | | |
| target site | | | | | |
| MG29-1 | 6082 | MG29-1-human | Nucleotide | N.A. | /AltR1/rUrArArUrUrUrCrUrArCrUrGrUrUrGrUrArGrArUrUrArUrUrGrUrUrCrCrUrCrU |
| sgRNA | | ANGPTL3-A1 | | | rArGrUrUrArUrUrUrCrC/AltR2/ |
| targeting | | | | | |
| human | | | | | |
| ANGPTL | | | | | |
| 3 | | | | | |
| MG29-1 | 6083 | MG29-1-human | Nucleotide | N.A. | /AltR1/rUrArArUrUrUrCrUrArCrUrGrUrUrGrUrArGrArUrArUrUrGrUrUrCrCrUrCrUrA |
| sgRNA | | ANGPTL3-B1 | | | rGrUrUrArUrUrUrCrCrU/AltR2/ |
| targeting | | | | | |
| human | | | | | |
| ANGPTL | | | | | |
| 3 | | | | | |
| MG29-1 | 6084 | MG29-1-human | Nucleotide | N.A. | /AltR1/rUrArArUrUrUrCrUrArCrUrGrUrUrGrUrArGrArUrUrUrGrUrUrCrCrUrCrUrArG |
| sgRNA | | ANGPTL3-C1 | | | rUrUrArUrUrUrCrCrUrC/AltR2/ |
| targeting | | | | | |
| human | | | | | |
| ANGPTL | | | | | |
| 3 | | | | | |
| MG29-1 | 6085 | MG29-1-human | Nucleotide | N.A. | /AltR1/rUrArArUrUrUrCrUrArCrUrGrUrUrGrUrArGrArUrCrUrCrCrArGrArArUrUrGrA |
| sgRNA | | ANGPTL3-D1 | | | rUrCrArArGrArCrArArU/AltR2/ |
| targeting | | | | | |
| human | | | | | |
| ANGPTL | | | | | |
| 3 | | | | | |
| MG29-1 | 6086 | MG29-1-human | Nucleotide | N.A. | /AltR1/rUrArArUrUrUrCrUrArCrUrGrUrUrGrUrArGrArUrArUrUrCrUrCrUrArUrCrUrC |
| sgRNA | | ANGPTL3-E1 | | | rCrArGrArGrCrCrArArA/AltR2/ |
| targeting | | | | | |
| human | | | | | |
| ANGPTL | | | | | |
| 3 | | | | | |
| MG29-1 | 6087 | MG29-1-human | Nucleotide | N.A. | /AltR1/rUrArArUrUrUrCrUrArCrUrGrUrUrGrUrArGrArUrArGrCrCrArArUrGrGrCrCrU |
| sgRNA | | ANGPTL3-F1 | | | rCrCrUrUrCrArGrUrUrG/AltR2/ |
| targeting | | | | | |
| human | | | | | |
| ANGPTL | | | | | |
| 3 | | | | | |
| MG29-1 | 6088 | MG29-1-human | Nucleotide | N.A. | /AltR1/rUrArArUrUrUrCrUrArCrUrGrUrUrGrUrArGrArUrGrCrCrArArUrGrGrCrCrUrC |
| sgRNA | | ANGPTL3-G1 | | | rCrUrUrCrArGrUrUrGrG/AltR2/ |
| targeting | | | | | |
| human | | | | | |
| ANGPTL | | | | | |
| 3 | | | | | |
| MG29-1 | 6089 | MG29-1-human | Nucleotide | N.A. | /AltR1/rUrArArUrUrUrCrUrArCrUrGrUrUrGrUrArGrArUrUrCrCrArUrArArGrArCrGrA |
| sgRNA | | ANGPTL3-H1 | | | rArGrGrGrCrCrArArArU/AltR2/ |
| targeting | | | | | |
| human | | | | | |
| ANGPTL | | | | | |
| 3 | | | | | |
| MG29-1 | 6090 | MG29-1-human | Nucleotide | N.A. | /AltR1/rUrArArUrUrUrCrUrArCrUrGrUrUrGrUrArGrArUrArArArArArCrUrCrArArCrA |
| sgRNA | | ANGPTL3-A2 | | | rUrArUrUrUrGrArUrCrA/AltR2/ |
| targeting | | | | | |
| human | | | | | |
| ANGPTL | | | | | |
| 3 | | | | | |
| MG29-1 | 6091 | MG29-1-human | Nucleotide | N.A. | /AltR1/rUrArArUrUrUrCrUrArCrUrGrUrUrGrUrArGrArUrArUrCrArGrUrCrUrUrUrUrU |
| sgRNA | | ANGPTL3-B2 | | | rArUrGrArUrCrUrArUrC/AltR2/ |
| targeting | | | | | |
| human | | | | | |
| ANGPTL | | | | | |
| 3 | | | | | |
| MG29-1 | 6092 | MG29-1-human | Nucleotide | N.A. | /AltR1/rUrArArUrUrUrCrUrArCrUrGrUrUrGrUrArGrArUrUrArUrGrArUrCrUrArUrCrG |
| sgRNA | | ANGPTL3-C2 | | | rCrUrGrCrArArArCrCrA/AltR2/ |
| targeting | | | | | |
| human | | | | | |
| ANGPTL | | | | | |
| 3 | | | | | |
| MG29-1 | 6093 | MG29-1-human | Nucleotide | N.A. | /AltR1/rUrArArUrUrUrCrUrArCrUrGrUrUrGrUrArGrArUrArUrGrArUrCrUrArUrCrGrC |
| sgRNA | | ANGPTL3-D2 | | | rUrGrCrArArArCrCrArG/AltR2/ |
| targeting | | | | | |
| human | | | | | |
| ANGPTL | | | | | |
| 3 | | | | | |
| MG29-1 | 6094 | MG29-1-human | Nucleotide | N.A. | /AltR1/rUrArArUrUrUrCrUrArCrUrGrUrUrGrUrArGrArUrUrGrArUrCrUrArUrCrGrCrU |
| sgRNA | | ANGPTL3-E2 | | | rGrCrArArArCrCrArGrU/AltR2/ |
| targeting | | | | | |
| human | | | | | |
| ANGPTL | | | | | |
| 3 | | | | | |
| MG29-1 | 6095 | MG29-1-human | Nucleotide | N.A. | /AltR1/rUrArArUrUrUrCrUrArCrUrGrUrUrGrUrArGrArUrGrArArGrArGrCrArArCrUrA |
| sgRNA | | ANGPTL3-F2 | | | rArCrUrArArCrUrUrArA/AltR2/ |
| targeting | | | | | |
| human | | | | | |
| ANGPTL | | | | | |
| 3 | | | | | |
| MG29-1 | 6096 | MG29-1-human | Nucleotide | N.A. | /AltR1/rUrArArUrUrUrCrUrArCrUrGrUrUrGrUrArGrArUrArGrUrGrArArGrUrUrArCrU |
| sgRNA | | ANGPTL3-G2 | | | rUrCrUrGrGrGrUrGrUrU/AltR2/ |
| targeting | | | | | |
| human | | | | | |
| ANGPTL | | | | | |
| 3 | | | | | |
| MG29-1 | 6097 | MG29-1-human | Nucleotide | N.A. | /AltR1/rUrArArUrUrUrCrUrArCrUrGrUrUrGrUrArGrArUrGrArArUrUrArArGrUrUrArG |
| sgRNA | | ANGPTL3-H2 | | | rUrUrArGrUrUrGrCrUrC/AltR2/ |
| targeting | | | | | |
| human | | | | | |
| ANGPTL | | | | | |
| 3 | | | | | |
| MG29-1 | 6098 | MG29-1-human | Nucleotide | N.A. | /AltR1/rUrArArUrUrUrCrUrArCrUrGrUrUrGrUrArGrArUrUrUrCrUrUrCrUrArGrGrArG |
| sgRNA | | ANGPTL3-A3 | | | rGrCrUrUrUrCrArArGrU/AltR2/ |
| targeting | | | | | |
| human | | | | | |
| ANGPTL | | | | | |
| 3 | | | | | |
| MG29-1 | 6099 | MG29-1-human | Nucleotide | N.A. | /AltR1/rUrArArUrUrUrCrUrArCrUrGrUrUrGrUrArGrArUrUrCrUrUrCrUrArGrGrArGrG |
| syRNA | | ANGPTL3-B3 | | | rCrUrUrUrCrArArGrUrU/AltR2/ |
| targeting | | | | | |
| human | | | | | |
| ANGPTL | | | | | |
| 3 | | | | | |
| MG29-1 | 6100 | MG29-1-human | Nucleotide | N.A. | /AltR1/rUrArArUrUrUrCrUrArCrUrGrUrUrGrUrArGrArUrCrUrUrCrUrArGrGrArGrGrC |
| sgRNA | | ANGPTL3-C3 | | | rUrUrUrCrArArGrUrUrU/AltR2/ |
| targeting | | | | | |
| human | | | | | |
| ANGPTL | | | | | |
| 3 | | | | | |
| MG29-1 | 6101 | MG29-1-human | Nucleotide | N.A. | /AltR1/rUrArArUrUrUrCrUrArCrUrGrUrUrGrUrArGrArUrUrUrCrUrArGrGrArGrGrCrU |
| sgRNA | | ANGPTL3-D3 | | | rUrUrCrArArGrUrUrUrU/AltR2/ |
| targeting | | | | | |
| human | | | | | |
| ANGPTL | | | | | |
| 3 | | | | | |
| MG29-1 | 6102 | MG29-1-human | Nucleotide | N.A. | /AltR1/rUrArArUrUrUrCrUrArCrUrGrUrUrGrUrArGrArUrArArGrUrUrUrUrGrArGrUrU |
| sgRNA | | ANGPTL3-E3 | | | rGrArGrUrUrCrArArGrU/AltR2/ |
| targeting | | | | | |
| human | | | | | |
| ANGPTL | | | | | |
| 3 | | | | | |
| MG29-1 | 6103 | MG29-1-human | Nucleotide | N.A. | /AltR1/rUrArArUrUrUrCrUrArCrUrGrUrUrGrUrArGrArUrGrArGrUrUrGrArGrUrUrCrA |
| sgRNA | | ANGPTL3-F3 | | | rArGrUrGrArCrArUrArU/AltR2/ |
| targeting | | | | | |
| human | | | | | |
| ANGPTL | | | | | |
| 3 | | | | | |
| MG29-1 | 6104 | MG29-1-human | Nucleotide | N.A. | /AltR1/rUrArArUrUrUrCrUrArCrUrGrUrUrGrUrArGrArUrArGrUrUrGrArGrUrUrCrArA |
| sgRNA | | ANGPTL3-G3 | | | rGrUrGrArCrArUrArUrU/AltR2/ |
| targeting | | | | | |
| human | | | | | |
| ANGPTL | | | | | |
| 3 | | | | | |
| MG29-1 | 6105 | MG29-1-human | Nucleotide | N.A. | /AltR1/rUrArArUrUrUrCrUrArCrUrGrUrUrGrUrArGrArUrCrCrUrCrUrUrCrArUrUrUrU |
| sgRNA | | ANGPTL3-H3 | | | rUrGrArCrUrUrGrUrArG/AltR2/ |
| targeting | | | | | |
| human | | | | | |
| ANGPTL | | | | | |
| 3 | | | | | |
| MG29-1 | 6106 | MG29-1-human | Nucleotide | N.A. | /AltR1/rUrArArUrUrUrCrUrArCrUrGrUrUrGrUrArGrArUrUrArUrGrUrArGrUrUrCrUrU |
| sgRNA | | ANGPTL3-A4 | | | rCrUrCrArGrUrUrCrCrU/AltR2/ |
| targeting | | | | | |
| human | | | | | |
| ANGPTL | | | | | |
| 3 | | | | | |
| MG29-1 | 6107 | MG29-1-human | Nucleotide | N.A. | /AltR1/rUrArArUrUrUrCrUrArCrUrGrUrUrGrUrArGrArUrCrUrUrCrUrUrCrUrUrUrGrA |
| sgRNA | | ANGPTL3-B4 | | | rUrUrUrCrArCrUrGrGrU/AltR2/ |
| targeting | | | | | |
| human | | | | | |
| ANGPTL | | | | | |
| 3 | | | | | |
| MG29-1 | 6108 | MG29-1-human | Nucleotide | N.A. | /AltR1/rUrArArUrUrUrCrUrArCrUrGrUrUrGrUrArGrArUrUrUrCrUrUrCrUrUrUrGrArU |
| sgRNA | | ANGPTL3-C4 | | | rUrUrCrArCrUrGrGrUrU/AltR2/ |
| targeting | | | | | |
| human | | | | | |
| ANGPTL | | | | | |
| 3 | | | | | |
| MG29-1 | 6109 | MG29-1-human | Nucleotide | N.A. | /AltR1/rUrArArUrUrUrCrUrArCrUrGrUrUrGrUrArGrArUrArUrUrUrCrArCrUrGrGrUrU |
| sgRNA | | ANGPTL3-D4 | | | rUrGrCrArGrCrGrArUrA/AltR2/ |
| targeting | | | | | |
| human | | | | | |
| ANGPTL | | | | | |
| 3 | | | | | |
| MG29-1 | 6110 | MG29-1-human | Nucleotide | N.A. | /AltR1/rUrArArUrUrUrCrUrArCrUrGrUrUrGrUrArGrArUrArCrUrGrGrUrUrUrGrCrArG |
| sgRNA | | ANGPTL3-E4 | | | rCrGrArUrArGrArUrCrA/AltR2/ |
| targeting | | | | | |
| human | | | | | |
| ANGPTL | | | | | |
| 3 | | | | | |
| MG29-1 | 6111 | MG29-1-human | Nucleotide | N.A. | /AltR1/rUrArArUrUrUrCrUrArCrUrGrUrUrGrUrArGrArUrCrArGrCrGrArUrArGrArUrC |
| syRNA | | ANGPTL3-F4 | | | rArUrArArArArArGrArC/AltR2/ |
| targeting | | | | | |
| human | | | | | |
| ANGPTL | | | | | |
| 3 | | | | | |
| MG29-1 | 6112 | MG29-1-human | Nucleotide | N.A. | /AltR1/rUrArArUrUrUrCrUrArCrUrGrUrUrGrUrArGrArUrGrArArArUrArUrGrUrCrArU |
| sgRNA | | ANGPTL3-G4 | | | UrUrArArrUrUrGrGrCrC/AltR2/ |
| targeting | | | | | |
| human | | | | | |
| ANGPTL | | | | | |
| 3 | | | | | |
| MG29-1 | 6113 | MG29-1-human | Nucleotide | N.A. | /AltR1/rUrArArUrUrUrCrUrArCrUrGrUrUrGrUrArGrArUrArArArUrArUrGrUrCrArUrU |
| sgRNA | | ANGPTL3-H4 | | | rArArUrUrUrGrGrCrCrC/AltR2/ |
| targeting | | | | | |
| human | | | | | |
| ANGPTL | | | | | |
| 3 | | | | | |
| MG29-1 | 6114 | MG29-1-human | Nucleotide | N.A. | /AltR1/rUrArArUrUrUrCrUrArCrUrGrUrUrGrUrArGrArUrGrCrCrCrUrUrCrGrUrCrUrU |
| sgRNA | | ANGPTL3-A5 | | | rArUrGrGrArCrArArArG/AltR2/ |
| targeting | | | | | |
| human | | | | | |
| ANGPTL | | | | | |
| 3 | | | | | |
| MG29-1 | 6115 | MG29-1-human | Nucleotide | N.A. | /AltR1/rUrArArUrUrUrCrUrArCrUrGrUrUrGrUrArGrArUrArGrArCrCrArUrGrUrCrCrC |
| sgRNA | | ANGPTL3-B5 | | | rArArCrUrGrArArGrGrA/AltR2/ |
| targeting | | | | | |
| human | | | | | |
| ANGPTL | | | | | |
| 3 | | | | | |
| MG29-1 | 6116 | MG29-1-human | Nucleotide | N.A. | /AltR1/rUrArArUrUrUrCrUrArCrUrGrUrUrGrUrArGrArUrUrArCrArUrCrGrUrCrUrArA |
| sgRNA | | ANGPTL3-C5 | | | rCrArUrArGrCrArArArU/AltR2/ |
| targeting | | | | | |
| human | | | | | |
| ANGPTL | | | | | |
| 3 | | | | | |
| MG29-1 | 6117 | MG29-1-human | Nucleotide | N.A. | /AltR1/rUrArArUrUrUrCrUrArCrUrGrUrUrGrUrArGrArUrArCrArUrCrGrUrCrUrArArC |
| sgRNA | | ANGPTL3-D5 | | | rArUrArGrCrArArArUrC/AltR2/ |
| targeting | | | | | |
| human | | | | | |
| ANGPTL | | | | | |
| 3 | | | | | |
| MG29-1 | 6118 | MG29-1-human | Nucleotide | N.A. | /AltR1/rUrArArUrUrUrCrUrArCrUrGrUrUrGrUrArGrArUrCrArUrCrGrUrCrUrArArCrA |
| sgRNA | | ANGPTL3-E5 | | | rUrArGrCrArArArUrCrU/AltR2/ |
| targeting | | | | | |
| human | | | | | |
| ANGPTL | | | | | |
| 3 | | | | | |
| MG29-1 | 6119 | MG29-1-human | Nucleotide | N.A. | /AltR1/rUrArArUrUrUrCrUrArCrUrGrUrUrGrUrArGrArUrGrGrCrUrCrUrGrGrArGrArU |
| sgRNA | | ANGPTL3-F5 | | | rArGrArGrArArUrCrArA/AltR2/ |
| targeting | | | | | |
| human | | | | | |
| ANGPTL | | | | | |
| 3 | | | | | |
| MG29-1 | 6120 | MG29-1-human | Nucleotide | N.A. | /AltR1/rUrArArUrUrUrCrUrArCrUrGrUrUrGrUrArGrArUrGrCrUrCrUrGrGrArGrArUrA |
| sgRNA | | ANGPTL3-G5 | | | rGrArGrArArUrCrArArA/AltR2/ |
| targeting | | | | | |
| human | | | | | |
| ANGPTL | | | | | |
| 3 | | | | | |
| MG29-1 | 6121 | MG29-1-human | Nucleotide | N.A. | /AltR1/rUrArArUrUrUrCrUrArCrUrGrUrUrGrUrArGrArUrUrUrUrUrArUrUrUrGrArCrU |
| sgRNA | | ANGPTL3-H5 | | | rArUrGrCrUrGrUrUrGrG/AltR2/ |
| targeting | | | | | |
| human | | | | | |
| ANGPTL | | | | | |
| 3 | | | | | |
| MG29-1 | 6122 | MG29-1-human | Nucleotide | N.A. | /AltR1/rUrArArUrUrUrCrUrArCrUrGrUrUrGrUrArGrArUrArUrUrUrGrArCrUrArUrGrC |
| sgRNA | | ANGPTL3-A6 | | | rUrGrUrUrGrGrUrUrUrA/AltR2/ |
| targeting | | | | | |
| human | | | | | |
| ANGPTL | | | | | |
| 3 | | | | | |
| MG29-1 | 6123 | MG29-1-human | Nucleotide | N.A. | /AltR1/rUrArArUrUrUrCrUrArCrUrGrUrUrGrUrArGrArUrUrUrUrGrArCrUrArUrGrCrU |
| sgRNA | | ANGPTL3-B6 | | | rGrUrUrGrGrUrUrUrArA/AltR2/ |
| targeting | | | | | |
| human | | | | | |
| ANGPTL | | | | | |
| 3 | | | | | |
| MG29-1 | 6124 | MG29-1-human | Nucleotide | N.A. | /AltR1/rUrArArUrUrUrCrUrArCrUrGrUrUrGrUrArGrArUrArCrUrArUrGrCrUrGrUrUrG |
| sgRNA | | ANGPTL3-C6 | | | rGrUrUrUrArArUrUrGrU/AltR2/ |
| targeting | | | | | |
| human | | | | | |
| ANGPTL | | | | | |
| 3 | | | | | |
| MG29-1 | 6125 | MG29-1-human | Nucleotide | N.A. | /AltR1/rUrArArUrUrUrCrUrArCrUrGrUrUrGrUrArGrArUrArUrUrGrUrUrUrArUrArUrU |
| sgRNA | | ANGPTL3-D6 | | | rGrGrUrCrUrUrCrCrArC/AltR2/ |
| targeting | | | | | |
| human | | | | | |
| ANGPTL | | | | | |
| 3 | | | | | |
| MG29-1 | 6126 | MG29-1-human | Nucleotide | N.A. | /AltR1/rUrArArUrUrUrCrUrArCrUrGrUrUrGrUrArGrArUrUrArUrUrGrGrUrCrUrUrCrC |
| sgRNA | | ANGPTL3-E6 | | | rArCrGrGrUrCrUrGrGrA/AltR2/ |
| targeting | | | | | |
| human | | | | | |
| ANGPTL | | | | | |
| 3 | | | | | |
| MG29-1 | 6127 | MG29-1-human | Nucleotide | N.A. | /AltR1/rUrArArUrUrUrCrUrArCrUrGrUrUrGrUrArGrArUrUrArUrUrCrUrUrUrUrArUrC |
| sgRNA | | ANGPTL3-F6 | | | rArGrCrUrCrArGrArArG/AltR2/ |
| targeting | | | | | |
| human | | | | | |
| ANGPTL | | | | | |
| 3 | | | | | |
| MG29-1 | 6128 | MG29-1-human | Nucleotide | N.A. | /AltR1/rUrArArUrUrUrCrUrArCrUrGrUrUrGrUrArGrArUrArUrCrArGrCrUrCrArGrArA |
| sgRNA | | ANGPTL3-G6 | | | CrGrGrArrUrArGrUrArU/AltR2/ |
| targeting | | | | | |
| human | | | | | |
| ANGPTL | | | | | |
| 3 | | | | | |
| MG29-1 | 6129 | MG29-1-human | Nucleotide | N.A. | /AltR1/rUrArArUrUrUrCrUrArCrUrGrUrUrGrUrArGrArUrUrCrArGrCrUrCrArGrArArG |
| sgRNA | | ANGPTL3-H6 | | | rGrArCrUrArGrUrArUrU/AltR2/ |
| targeting | | | | | |
| human | | | | | |
| ANGPTL | | | | | |
| 3 | | | | | |
| MG29-1 | 6130 | MG29-1-human | Nucleotide | N.A. | /AltR1/rUrArArUrUrUrCrUrArCrUrGrUrUrGrUrArGrArUrUrCrUrArUrCrUrUrCrCrArA |
| sgRNA | | ANGPTL3-A7 | | | rGrCrCrArArGrArGrCrA/AltR2/ |
| targeting | | | | | |
| human | | | | | |
| ANGPTL | | | | | |
| 3 | | | | | |
| MG29-1 | 6131 | MG29-1-human | Nucleotide | N.A. | /AltR1/rUrArArUrUrUrCrUrArCrUrGrUrUrGrUrArGrArUrUrGrUrGrGrGrUrUrCrUrUrG |
| sgRNA | | ANGPTL3-B7 | | | rArArUrArCrUrArGrUrC/AltR2/ |
| targeting | | | | | |
| human | | | | | |
| ANGPTL | | | | | |
| 3 | | | | | |
| MG29-1 | 6132 | MG29-1-human | Nucleotide | N.A. | /AltR1/rUrArArUrUrUrCrUrArCrUrGrUrUrGrUrArGrArUrArUrUrGrArUrUrCrUrArGrG |
| sgRNA | | ANGPTL3-C7 | | | rCrArUrUrCrCrUrGrCrU/AltR2/ |
| targeting | | | | | |
| human | | | | | |
| ANGPTL | | | | | |
| 3 | | | | | |
| MG29-1 | 6133 | MG29-1-human | Nucleotide | N.A. | /AltR1/rUrArArUrUrUrCrUrArCrUrGrUrUrGrUrArGrArUrUrUrGrArUrUrCrUrArGrGrC |
| sgRNA | | ANGPTL3-D7 | | | rArUrUrCrCrUrGrCrUrG/AltR2/ |
| targeting | | | | | |
| human | | | | | |
| ANGPTL | | | | | |
| 3 | | | | | |
| MG29-1 | 6134 | MG29-1-human | Nucleotide | N.A. | /AltR1/rUrArArUrUrUrCrUrArCrUrGrUrUrGrUrArGrArUrUrArArCrArGrArGrGrUrGrA |
| sgRNA | | ANGPTL3-E7 | | | rArCrArUrArCrArArGrU/AltR2/ |
| targeting | | | | | |
| human | | | | | |
| ANGPTL | | | | | |
| 3 | | | | | |
| MG29-1 | 6135 | MG29-1-human | Nucleotide | N.A. | /AltR1/rUrArArUrUrUrCrUrArCrUrGrUrUrGrUrArGrArUrUrCrArUrGrUrCrUrArCrUrG |
| sgRNA | | ANGPTL3-F7 | | | rUrGrArUrGrUrUrArUrA/AltR2/ |
| targeting | | | | | |
| human | | | | | |
| ANGPTL | | | | | |
| 3 | | | | | |
| MG29-1 | 6136 | MG29-1-human | Nucleotide | N.A. | /AltR1/rUrArArUrUrUrCrUrArCrUrGrUrUrGrUrArGrArUrCrArUrGrUrCrUrArCrUrGrU |
| sgRNA | | ANGPTL3-G7 | | | rGrArUrGrUrUrArUrArU/AltR2/ |
| targeting | | | | | |
| human | | | | | |
| ANGPTL | | | | | |
| 3 | | | | | |
| MG29-1 | 6137 | MG29-1-human | Nucleotide | N.A. | /AltR1/rUrArArUrUrUrCrUrArCrUrGrUrUrGrUrArGrArUrArUrGrUrCrUrArCrUrGrUrG |
| sgRNA | | ANGPTL3-H7 | | | rArUrGrUrUrArUrArUrC/AltR2/ |
| targeting | | | | | |
| human | | | | | |
| ANGPTL | | | | | |
| 3 | | | | | |
| MG29-1 | 6138 | MG29-1-human | Nucleotide | N.A. | /AltR1/rUrArArUrUrUrCrUrArCrUrGrUrUrGrUrArGrArUrArCrCrUrGrArUrArUrArArC |
| sgRNA | | ANGPTL3-A8 | | | rArUrCrArCrArGrUrArG/AltR2/ |
| targeting | | | | | |
| human | | | | | |
| ANGPTL | | | | | |
| 3 | | | | | |
| MG29-1 | 6139 | MG29-1-human | Nucleotide | N.A. | /AltR1/rUrArArUrUrUrCrUrArCrUrGrUrUrGrUrArGrArUrCrCrUrGrArUrArUrArArCrA |
| sgRNA | | ANGPTL3-B8 | | | rUrCrArCrArGrUrArGrA/AltR2/ |
| targeting | | | | | |
| human | | | | | |
| ANGPTL | | | | | |
| 3 | | | | | |
| MG29-1 | 6140 | MG29-1-human | Nucleotide | N.A. | /AltR1/rUrArArUrUrUrCrUrArCrUrGrUrUrGrUrArGrArUrArUrUrCrArUrUrArUrArUrU |
| sgRNA | | ANGPTL3-C8 | | | rCrArGrGrUrArGrUrCrC/AltR2/ |
| targeting | | | | | |
| human | | | | | |
| ANGPTL | | | | | |
| 3 | | | | | |
| MG29-1 | 6141 | MG29-1-human | Nucleotide | N.A. | /AltR1/rUrArArUrUrUrCrUrArCrUrGrUrUrGrUrArGrArUrUrArGrUrUrCrUrCrCrCrArC |
| sgRNA | | ANGPTL3-D8 | | | rGrUrUrUrCrArUrUrGrA/AltR2/ |
| targeting | | | | | |
| human | | | | | |
| ANGPTL | | | | | |
| 3 | | | | | |
| MG29-1 | 6142 | MG29-1-human | Nucleotide | N.A. | /AltR1/rUrArArUrUrUrCrUrArCrUrGrUrUrGrUrArGrArUrArUrUrGrArArGrUrUrUrUrG |
| sgRNA | | ANGPTL3-E8 | | | rUrGrArUrCrCrArUrCrU/AltR2/ |
| targeting | | | | | |
| human | | | | | |
| ANGPTL | | | | | |
| 3 | | | | | |
| MG29-1 | 6143 | MG29-1-human | Nucleotide | N.A. | /AltR1/rUrArArUrUrUrCrUrArCrUrGrUrUrGrUrArGrArUrGrUrGrArUrCrCrArUrCrUrA |
| sgRNA | | ANGPTL3-F8 | | | rUrUrCrGrArUrGrUrUrG/AltR2/ |
| targeting | | | | | |
| human | | | | | |
| ANGPTL | | | | | |
| 3 | | | | | |
| MG29-1 | 6144 | MG29-1-human | Nucleotide | N.A. | /AltR1/rUrArArUrUrUrCrUrArCrUrGrUrUrGrUrArGrArUrUrGrArUrCrCrArUrCrUrArU |
| sgRNA | | ANGPTL3-G8 | | | rUrCrGrArUrGrUrUrGrA/AltR2/ |
| targeting | | | | | |
| human | | | | | |
| ANGPTL | | | | | |
| 3 | | | | | |
| MG29-1 | 6145 | MG29-1-human | Nucleotide | N.A. | /AltR1/rUrArArUrUrUrCrUrArCrUrGrUrUrGrUrArGrArUrCrUrUrUrUrCrArGrGrArGrA |
| sgRNA | | ANGPTL3-H8 | | | rArUrUrUrUrGrGrUrUrG/AltR2/ |
| targeting | | | | | |
| human | | | | | |
| ANGPTL | | | | | |
| 3 | | | | | |
| MG29-1 | 6146 | MG29-1-human | Nucleotide | N.A. | /AltR1/rUrArArUrUrUrCrUrArCrUrGrUrUrGrUrArGrArUrUrUrUrUrCrArGrGrArGrArA |
| sgRNA | | ANGPTL3-A9 | | | rUrUrUrUrGrGrUrUrGrG/AltR2/ |
| targeting | | | | | |
| human | | | | | |
| ANGPTL | | | | | |
| 3 | | | | | |
| MG29-1 | 6147 | MG29-1-human | Nucleotide | N.A. | /AltR1/rUrArArUrUrUrCrUrArCrUrGrUrUrGrUrArGrArUrCrArGrGrArGrArArUrUrUrU |
| sgRNA | | ANGPTL3-B9 | | | rGrGrUrUrGrGrGrCrCrU/AltR2/ |
| targeting | | | | | |
| human | | | | | |
| ANGPTL | | | | | |
| 3 | | | | | |
| MG29-1 | 6148 | MG29-1-human | Nucleotide | N.A. | /AltR1/rUrArArUrUrUrCrUrArCrUrGrUrUrGrUrArGrArUrArGrGrArGrArArUrUrUrUrG |
| sgRNA | | ANGPTL3-C9 | | | rGrUrUrGrGrGrCrCrUrA/AltR2/ |
| targeting | | | | | |
| human | | | | | |
| ANGPTL | | | | | |
| 3 | | | | | |
| MG29-1 | 6149 | MG29-1-human | Nucleotide | N.A. | /AltR1/rUrArArUrUrUrCrUrArCrUrGrUrUrGrUrArGrArUrGrGrUrUrGrGrGrCrCrUrArG |
| sgRNA | | ANGPTL3-D9 | | | rArGrArArGrArUrArUrA/AltR2/ |
| targeting | | | | | |
| human | | | | | |
| ANGPTL | | | | | |
| 3 | | | | | |
| MG29-1 | 6150 | MG29-1-human | Nucleotide | N.A. | /AltR1/rUrArArUrUrUrCrUrArCrUrGrUrUrGrUrArGrArUrGrUrUrGrGrGrCrCrUrArGrA |
| sgRNA | | ANGPTL3-E9 | | | rGrArArGrArUrArUrArC/AltR2/ |
| targeting | | | | | |
| human | | | | | |
| ANGPTL | | | | | |
| 3 | | | | | |
| MG29-1 | 6151 | MG29-1-human | Nucleotide | N.A. | /AltR1/rUrArArUrUrUrCrUrArCrUrGrUrUrGrUrArGrArUrArCrGrArArUrUrGrArGrUrU |
| sgRNA | | ANGPTL3-F9 | | | rGrGrArArGrArCrUrGrG/AltR2/ |
| targeting | | | | | |
| human | | | | | |
| ANGPTL | | | | | |
| 3 | | | | | |
| MG29-1 | 6152 | MG29-1-human | Nucleotide | N.A. | /AltR1/rUrArArUrUrUrCrUrArCrUrGrUrUrGrUrArGrArUrCrGrArArUrUrGrArGrUrUrG |
| sgRNA | | ANGPTL3-G9 | | | rGrArArGrArCrUrGrGrA/AltR2/ |
| targeting | | | | | |
| human | | | | | |
| ANGPTL | | | | | |
| 3 | | | | | |
| MG29-1 | 6153 | MG29-1-human | Nucleotide | N.A. | /AltR1/rUrArArUrUrUrCrUrArCrUrGrUrUrGrUrArGrArUrUrArCrUrUrGrGrGrArArArU |
| sgRNA | | ANGPTL3-H9 | | | rCrArCrGrArArArCrCrA/AltR2/ |
| targeting | | | | | |
| human | | | | | |
| ANGPTL | | | | | |
| 3 | | | | | |
| MG29-1 | 6154 | MG29-1-human | Nucleotide | N.A. | /AltR1/rUrArArUrUrUrCrUrArCrUrGrUrUrGrUrArGrArUrArCrUrUrGrGrGrArArArUrC |
| sgRNA | | ANGPTL3-A10 | | | rArCrGrArArArCrCrArA/AltR2/ |
| targeting | | | | | |
| human | | | | | |
| ANGPTL | | | | | |
| 3 | | | | | |
| MG29-1 | 6155 | MG29-1-human | Nucleotide | N.A. | /AltR1/rUrArArUrUrUrCrUrArCrUrGrUrUrGrUrArGrArUrCrUrUrGrGrGrArArArUrCrA |
| sgRNA | | ANGPTL3-B10 | | | rCrGrArArArCrCrArArC/AltR2/ |
| targeting | | | | | |
| human | | | | | |
| ANGPTL | | | | | |
| 3 | | | | | |
| MG29-1 | 6156 | MG29-1-human | Nucleotide | N.A. | /AltR1/rUrArArUrUrUrCrUrArCrUrGrUrUrGrUrArGrArUrGrUrGrUrUrUrUrCrUrArCrU |
| sgRNA | | ANGPTL3-C10 | | | rUrGrGrGrArUrCrArCrA/AltR2/ |
| targeting | | | | | |
| human | | | | | |
| ANGPTL | | | | | |
| 3 | | | | | |
| MG29-1 | 6157 | MG29-1-human | Nucleotide | N.A. | /AltR1/rUrArArUrUrUrCrUrArCrUrGrUrUrGrUrArGrArUrCrUrArCrUrUrGrGrGrArUrC |
| sgRNA | | ANGPTL3-D10 | | | rArCrArArArGrCrArArA/AltR2/ |
| targeting | | | | | |
| human | | | | | |
| ANGPTL | | | | | |
| 3 | | | | | |
| MG29-1 | 6158 | MG29-1-human | Nucleotide | N.A. | /AltR1/rUrArArUrUrUrCrUrArCrUrGrUrUrGrUrArGrArUrUrArCrUrUrGrGrGrArUrCrA |
| sgRNA | | ANGPTL3-E10 | | | rCrArArArGrCrArArArA/AltR2/ |
| targeting | | | | | |
| human | | | | | |
| ANGPTL | | | | | |
| 3 | | | | | |
| MG29-1 | 6159 | MG29-1-human | Nucleotide | N.A. | /AltR1/rUrArArUrUrUrCrUrArCrUrGrUrUrGrUrArGrArUrGrCrUrUrUrGrUrGrArUrCrC |
| sgRNA | | ANGPTL3-F10 | | | rCrArArGrUrArGrArArA/AltR2/ |
| targeting | | | | | |
| human | | | | | |
| ANGPTL | | | | | |
| 3 | | | | | |
| MG29-1 | 6160 | MG29-1-human | Nucleotide | N.A. | /AltR1/rUrArArUrUrUrCrUrArCrUrGrUrUrGrUrArGrArUrCrUrUrUrGrUrGrArUrCrCrC |
| sgRNA | | ANGPTL3-G10 | | | rArArGrUrArGrArArArA/AltR2/ |
| targeting | | | | | |
| human | | | | | |
| ANGPTL | | | | | |
| 3 | | | | | |
| MG29-1 | 6161 | MG29-1-human | Nucleotide | N.A. | /AltR1/rUrArArUrUrUrCrUrArCrUrGrUrUrGrUrArGrArUrUrGrArUrCrCrCrArArGrUrA |
| sgRNA | | ANGPTL3-H10 | | | rGrArArArArCrArCrCrA/AltR2/ |
| targeting | | | | | |
| human | | | | | |
| ANGPTL | | | | | |
| 3 | | | | | |
| MG29-1 | 6162 | MG29-1-human | Nucleotide | N.A. | /AltR1/rUrArArUrUrUrCrUrArCrUrGrUrUrGrUrArGrArUrUrUrUrUrCrCrGrGrGrArUrU |
| sgRNA | | ANGPTL3-A11 | | | rGrCrArUrUrGrGrGrGrA/AltR2/ |
| targeting | | | | | |
| human | | | | | |
| ANGPTL | | | | | |
| 3 | | | | | |
| MG29-1 | 6163 | MG29-1-human | Nucleotide | N.A. | /AltR1/rUrArArUrUrUrCrUrArCrUrGrUrUrGrUrArGrArUrCrCrGrGrGrArUrUrGrCrArU |
| sgRNA | | ANGPTL3-B11 | | | rUrGrGrGrGrArCrArUrU/AltR2/ |
| targeting | | | | | |
| human | | | | | |
| ANGPTL | | | | | |
| 3 | | | | | |
| MG29-1 | 6164 | MG29-1-human | Nucleotide | N.A. | /AltR1/rUrArArUrUrUrCrUrArCrUrGrUrUrGrUrArGrArUrCrGrGrGrArUrUrGrCrArUrU |
| sgRNA | | ANGPTL3-C11 | | | rGrGrGrGrArCrArUrUrG/AltR2/ |
| targeting | | | | | |
| human | | | | | |
| ANGPTL | | | | | |
| 3 | | | | | |
| MG29-1 | 6165 | MG29-1-human | Nucleotide | N.A. | /AltR1/rUrArArUrUrUrCrUrArCrUrGrUrUrGrUrArGrArUrGrUrGrArUrUrUrCrCrCrArA |
| sgRNA | | ANGPTL3-D11 | | | rGrUrArArArArArGrArA/AltR2/ |
| targeting | | | | | |
| human | | | | | |
| ANGPTL | | | | | |
| 3 | | | | | |
| MG29-1 | 6166 | MG29-1-human | Nucleotide | N.A. | /AltR1/rUrArArUrUrUrCrUrArCrUrGrUrUrGrUrArGrArUrUrUrGrUrCrUrUrUrCrCrArG |
| sgRNA | | ANGPTL3-E11 | | | rUrCrUrUrCrCrArArCrU/AltR2/ |
| targeting | | | | | |
| human | | | | | |
| ANGPTL | | | | | |
| 3 | | | | | |
| MG29-1 | 6167 | MG29-1-human | Nucleotide | N.A. | /AltR1/rUrArArUrUrUrCrUrArCrUrGrUrUrGrUrArGrArUrCrArGrUrCrUrUrCrCrArArC |
| sgRNA | | ANGPTL3-F11 | | | rUrCrArArUrUrCrGrUrA/AltR2/ |
| targeting | | | | | |
| human | | | | | |
| ANGPTL | | | | | |
| 3 | | | | | |
| MG29-1 | 6168 | MG29-1-human | Nucleotide | N.A. | /AltR1/rUrArArUrUrUrCrUrArCrUrGrUrUrGrUrArGrArUrGrGrArGrGrCrUrGrGrUrGrG |
| sgRNA | | ANGPTL3-G11 | | | rUrGrGrCrArUrGrArUrG/AltR2/ |
| targeting | | | | | |
| human | | | | | |
| ANGPTL | | | | | |
| 3 | | | | | |
| MG29-1 | 6169 | MG29-1-human | Nucleotide | N.A. | /AltR1/rUrArArUrUrUrCrUrArCrUrGrUrUrGrUrArGrArUrArArArUrUrUrGrCrCrUrCrA |
| sgRNA | | ANGPTL3-H11 | | | rGrUrUrCrArUrUrCrArA/AltR2/ |
| targeting | | | | | |
| human | | | | | |
| ANGPTL | | | | | |
| 3 | | | | | |
| MG29-1 | 6170 | MG29-1-human | Nucleotide | N.A. | /AltR1/rUrArArUrUrUrCrUrArCrUrGrUrUrGrUrArGrArUrArArUrUrUrGrCrCrUrCrArG |
| sgRNA | | ANGPTL3-A12 | | | rUrUrCrArUrUrCrArArA/AltR2/ |
| targeting | | | | | |
| human | | | | | |
| ANGPTL | | | | | |
| 3 | | | | | |
| MG29-1 | 6171 | MG29-1-human | Nucleotide | N.A. | /AltR1/rUrArArUrUrUrCrUrArCrUrGrUrUrGrUrArGrArUrCrCrUrCrArGrUrUrCrArUrU |
| sgRNA | | ANGPTL3-B12 | | | rCrArArArGrCrUrUrUrC/AltR2/ |
| targeting | | | | | |
| human | | | | | |
| ANGPTL | | | | | |
| 3 | | | | | |
| MG29-1 | 6172 | MG29-1-human | Nucleotide | N.A. | /AltR1/rUrArArUrUrUrCrUrArCrUrGrUrUrGrUrArGrArUrUrGrArArUrCrUrGrUrUrGrG |
| sgRNA | | ANGPTL3-C12 | | | rArUrGrGrArUrCrArArC/AltR2/ |
| targeting | | | | | |
| human | | | | | |
| ANGPTL | | | | | |
| 3 | | | | | |
| MG29-1 | 6173 | MG29-1-human | Nucleotide | N.A. | /AltR1/rUrArArUrUrUrCrUrArCrUrGrUrUrGrUrArGrArUrUrArGrArGrUrArUrArArCrC |
| sgRNA | | ANGPTL3-D12 | | | rUrUrCrCrArUrUrUrUrG/AltR2/ |
| targeting | | | | | |
| human | | | | | |
| ANGPTL | | | | | |
| 3 | | | | | |
| MG29-1 | 6174 | MG29-1-human | Nucleotide | N.A. | /AltR1/rUrArArUrUrUrCrUrArCrUrGrUrUrGrUrArGrArUrGrArGrArCrUrUrCrCrArArG |
| sgRNA | | ANGPTL3-E12 | | | rArUrArArUrCrCrUrCrU/AltR2/ |
| targeting | | | | | |
| human | | | | | |
| ANGPTL | | | | | |
| 3 | | | | | |
| MG29-1 | 6175 | MG29-1-human | Nucleotide | N.A. | /AltR1/rUrArArUrUrUrCrUrArCrUrGrUrUrGrUrArGrArUrArGrArCrUrUrCrCrArArGrA |
| sgRNA | | ANGPTL3-F12 | | | rUrArArUrCrCrUrCrUrU/AltR2/ |
| targeting | | | | | |
| human | | | | | |
| ANGPTL | | | | | |
| 3 | | | | | |
| MG29-1 | 6176 | MG29-1-human | Nucleotide | N.A. | /AltR1/rUrArArUrUrUrCrUrArCrUrGrUrUrGrUrArGrArUrGrCrUrCrUrUrGrGrUrUrUrG |
| sgRNA | | ANGPTL3-G12 | | | rUrUrArUrArUrUrUrArC/AltR2/ |
| targeting | | | | | |
| human | | | | | |
| ANGPTL | | | | | |
| 3 | | | | | |
| MG29-1 | 6177 | MG29-1-human | Nucleotide | N.A. | /AltR1/rUrArArUrUrUrCrUrArCrUrGrUrUrGrUrArGrArUrCrUrCrUrUrGrGrUrUrUrGrU |
| syRNA | | ANGPTL3-H12 | | | rUrArUrArUrUrUrArCrC/AltR2/ |
| targeting | | | | | |
| human | | | | | |
| ANGPTL | | | | | |
| 3 | | | | | |
| DNA | 6178 | MG29-1-human | Nucleotide | N.A. | TATTGTTCCTCTAGTTATTTCC |
| sequence | | ANGPTL3-A1 | | | |
| of human | | | | | |
| ANGPTL | | | | | |
| 3 target | | | | | |
| site | | | | | |
| DNA | 6179 | MG29-1-human | Nucleotide | N.A. | ATTGTTCCTCTAGTTATTTCCT |
| sequence | | ANGPTL3-B1 | | | |
| of human | | | | | |
| ANGPTL | | | | | |
| 3 target | | | | | |
| site | | | | | |
| DNA | 6180 | MG29-1-human | Nucleotide | N.A. | TTGTTCCTCTAGTTATTTCCTC |
| sequence | | ANGPTL3-C1 | | | |
| of human | | | | | |
| ANGPTL | | | | | |
| 3 target | | | | | |
| site | | | | | |
| DNA | 6181 | MG29-1-human | Nucleotide | N.A. | CTCCAGAATTGATCAAGACAAT |
| sequence | | ANGPTL3-D1 | | | |
| of human | | | | | |
| ANGPTL | | | | | |
| 3 target | | | | | |
| site | | | | | |
| DNA | 6182 | MG29-1-human | Nucleotide | N.A. | ATTCTCTATCTCCAGAGCCAAA |
| sequence | | ANGPTL3-E1 | | | |
| of human | | | | | |
| ANGPTL | | | | | |
| 3 target | | | | | |
| site | | | | | |
| DNA | 6183 | MG29-1-human | Nucleotide | N.A. | AGCCAATGGCCTCCTTCAGTTG |
| sequence | | ANGPTL3-F1 | | | |
| of human | | | | | |
| ANGPTL | | | | | |
| 3 target | | | | | |
| site | | | | | |
| DNA | 6184 | MG29-1-human | Nucleotide | N.A. | GCCAATGGCCTCCTTCAGTTGG |
| sequence | | ANGPTL3-G1 | | | |
| of human | | | | | |
| ANGPTL | | | | | |
| 3 target | | | | | |
| site | | | | | |
| DNA | 6185 | MG29-1-human | Nucleotide | N.A. | TCCATAAGACGAAGGGCCAAAT |
| sequence | | ANGPTL3-H1 | | | |
| of human | | | | | |
| ANGPTL | | | | | |
| 3 target | | | | | |
| site | | | | | |
| DNA | 6186 | MG29-1-human | Nucleotide | N.A. | AAAAACTCAACATATTTGATCA |
| sequence | | ANGPTL3-A2 | | | |
| of human | | | | | |
| ANGPTL | | | | | |
| 3 target | | | | | |
| site | | | | | |
| DNA | 6187 | MG29-1-human | Nucleotide | N.A. | ATCAGTCTTTTTATGATCTATC |
| sequence | | ANGPTL3-B2 | | | |
| of human | | | | | |
| ANGPTL | | | | | |
| 3 target | | | | | |
| site | | | | | |
| DNA | 6188 | MG29-1-human | Nucleotide | N.A. | TATGATCTATCGCTGCAAACCA |
| sequence | | ANGPTL3-C2 | | | |
| of human | | | | | |
| ANGPTL | | | | | |
| 3 target | | | | | |
| site | | | | | |
| DNA | 6189 | MG29-1-human | Nucleotide | N.A. | ATGATCTATCGCTGCAAACCAG |
| sequence | | ANGPTL3-D2 | | | |
| of human | | | | | |
| ANGPTL | | | | | |
| 3 target | | | | | |
| site | | | | | |
| DNA | 6190 | MG29-1-human | Nucleotide | N.A. | TGATCTATCGCTGCAAACCAGT |
| sequence | | ANGPTL3-E2 | | | |
| of human | | | | | |
| ANGPTL | | | | | |
| 3 target | | | | | |
| site | | | | | |
| DNA | 6191 | MG29-1-human | Nucleotide | N.A. | GAAGAGCAACTAACTAACTTAA |
| sequence | | ANGPTL3-F2 | | | |
| of human | | | | | |
| ANGPTL | | | | | |
| 3 target | | | | | |
| site | | | | | |
| DNA | 6192 | MG29-1-human | Nucleotide | N.A. | AGTGAAGTTACTTCTGGGTGTT |
| sequence | | ANGPTL3-G2 | | | |
| of human | | | | | |
| ANGPTL | | | | | |
| 3 target | | | | | |
| site | | | | | |
| DNA | 6193 | MG29-1-human | Nucleotide | N.A. | GAATTAAGTTAGTTAGTTGCTC |
| sequence | | ANGPTL3-H2 | | | |
| of human | | | | | |
| ANGPTL | | | | | |
| 3 target | | | | | |
| site | | | | | |
| DNA | 6194 | MG29-1-human | Nucleotide | N.A. | TTCTTCTAGGAGGCTTTCAAGT |
| sequence | | ANGPTL3-A3 | | | |
| of human | | | | | |
| ANGPTL | | | | | |
| 3 target | | | | | |
| site | | | | | |
| DNA | 6195 | MG29-1-human | Nucleotide | N.A. | TCTTCTAGGAGGCTTTCAAGTT |
| sequence | | ANGPTL3-B3 | | | |
| of human | | | | | |
| ANGPTL | | | | | |
| 3 target | | | | | |
| site | | | | | |
| DNA | 6196 | MG29-1-human | Nucleotide | N.A. | CTTCTAGGAGGCTTTCAAGTTT |
| sequence | | ANGPTL3-C3 | | | |
| of human | | | | | |
| ANGPTL | | | | | |
| 3 target | | | | | |
| site | | | | | |
| DNA | 6197 | MG29-1-human | Nucleotide | N.A. | TTCTAGGAGGCTTTCAAGTTTT |
| sequence | | ANGPTL3-D3 | | | |
| of human | | | | | |
| ANGPTL | | | | | |
| 3 target | | | | | |
| site | | | | | |
| DNA | 6198 | MG29-1-human | Nucleotide | N.A. | AAGTTTTGAGTTGAGTTCAAGT |
| sequence | | ANGPTL3-E3 | | | |
| of human | | | | | |
| ANGPTL | | | | | |
| 3 target | | | | | |
| site | | | | | |
| DNA | 6199 | MG29-1-human | Nucleotide | N.A. | GAGTTGAGTTCAAGTGACATAT |
| sequence | | ANGPTL3-F3 | | | |
| of human | | | | | |
| ANGPTL | | | | | |
| 3 target | | | | | |
| site | | | | | |
| DNA | 6200 | MG29-1-human | Nucleotide | N.A. | AGTTGAGTTCAAGTGACATATT |
| sequence | | ANGPTL3-G3 | | | |
| of human | | | | | |
| ANGPTL | | | | | |
| 3 target | | | | | |
| site | | | | | |
| DNA | 6201 | MG29-1-human | Nucleotide | N.A. | CCTCTTCATTTTTGACTTGTAG |
| sequence | | ANGPTL3-H3 | | | |
| of human | | | | | |
| ANGPTL | | | | | |
| 3 target | | | | | |
| site | | | | | |
| DNA | 6202 | MG29-1-human | Nucleotide | N.A. | TATGTAGTTCTTCTCAGTTCCT |
| sequence | | ANGPTL3-A4 | | | |
| of human | | | | | |
| ANGPTL | | | | | |
| 3 target | | | | | |
| site | | | | | |
| DNA | 6203 | MG29-1-human | Nucleotide | N.A. | CTTCTTCTTTGATTTCACTGGT |
| sequence | | ANGPTL3-B4 | | | |
| of human | | | | | |
| ANGPTL | | | | | |
| 3 target | | | | | |
| site | | | | | |
| DNA | 6204 | MG29-1-human | Nucleotide | N.A. | TTCTTCTTTGATTTCACTGGTT |
| sequence | | ANGPTL3-C4 | | | |
| of human | | | | | |
| ANGPTL | | | | | |
| 3 target | | | | | |
| site | | | | | |
| DNA | 6205 | MG29-1-human | Nucleotide | N.A. | ATTTCACTGGTTTGCAGCGATA |
| sequence | | ANGPTL3-D4 | | | |
| of human | | | | | |
| ANGPTL | | | | | |
| 3 target | | | | | |
| site | | | | | |
| DNA | 6206 | MG29-1-human | Nucleotide | N.A. | ACTGGTTTGCAGCGATAGATCA |
| sequence | | ANGPTL3-E4 | | | |
| of human | | | | | |
| ANGPTL | | | | | |
| 3 target | | | | | |
| site | | | | | |
| DNA | 6207 | MG29-1-human | Nucleotide | N.A. | CAGCGATAGATCATAAAAAGAC |
| sequence | | ANGPTL3-F4 | | | |
| of human | | | | | |
| ANGPTL | | | | | |
| 3 target | | | | | |
| site | | | | | |
| DNA | 6208 | MG29-1-human | Nucleotide | N.A. | GAAATATGTCATTAATTTGGCC |
| sequence | | ANGPTL3-G4 | | | |
| of human | | | | | |
| ANGPTL | | | | | |
| 3 target | | | | | |
| site | | | | | |
| DNA | 6209 | MG29-1-human | Nucleotide | N.A. | AAATATGTCATTAATTTGGCCC |
| sequence | | ANGPTL3-H4 | | | |
| of human | | | | | |
| ANGPTL | | | | | |
| 3 target | | | | | |
| site | | | | | |
| DNA | 6210 | MG29-1-human | Nucleotide | N.A. | GCCCTTCGTCTTATGGACAAAG |
| sequence | | ANGPTL3-A5 | | | |
| of human | | | | | |
| ANGPTL | | | | | |
| 3 target | | | | | |
| site | | | | | |
| DNA | 6211 | MG29-1-human | Nucleotide | N.A. | AGACCATGTCCCAACTGAAGGA |
| sequence | | ANGPTL3-B5 | | | |
| of human | | | | | |
| ANGPTL | | | | | |
| 3 target | | | | | |
| site | | | | | |
| DNA | 6212 | MG29-1-human | Nucleotide | N.A. | TACATCGTCTAACATAGCAAAT |
| sequence | | ANGPTL3-C5 | | | |
| of human | | | | | |
| ANGPTL | | | | | |
| 3 target | | | | | |
| site | | | | | |
| DNA | 6213 | MG29-1-human | Nucleotide | N.A. | ACATCGTCTAACATAGCAAATC |
| sequence | | ANGPTL3-D5 | | | |
| of human | | | | | |
| ANGPTL | | | | | |
| 3 target | | | | | |
| site | | | | | |
| DNA | 6214 | MG29-1-human | Nucleotide | N.A. | CATCGTCTAACATAGCAAATCT |
| sequence | | ANGPTL3-E5 | | | |
| of human | | | | | |
| ANGPTL | | | | | |
| 3 target | | | | | |
| site | | | | | |
| DNA | 6215 | MG29-1-human | Nucleotide | N.A. | GGCTCTGGAGATAGAGAATCAA |
| sequence | | ANGPTL3-F5 | | | |
| of human | | | | | |
| ANGPTL | | | | | |
| 3 target | | | | | |
| site | | | | | |
| DNA | 6216 | MG29-1-human | Nucleotide | N.A. | GCTCTGGAGATAGAGAATCAAA |
| sequence | | ANGPTL3-G5 | | | |
| of human | | | | | |
| ANGPTL | | | | | |
| 3 target | | | | | |
| site | | | | | |
| DNA | 6217 | MG29-1-human | Nucleotide | N.A. | TTTTATTTGACTATGCTGTTGG |
| sequence | | ANGPTL3-H5 | | | |
| of human | | | | | |
| ANGPTL | | | | | |
| 3 target | | | | | |
| site | | | | | |
| DNA | 6218 | MG29-1-human | Nucleotide | N.A. | ATTTGACTATGCTGTTGGTTTA |
| sequence | | ANGPTL3-A6 | | | |
| of human | | | | | |
| ANGPTL | | | | | |
| 3 target | | | | | |
| site | | | | | |
| DNA | 6219 | MG29-1-human | Nucleotide | N.A. | TTTGACTATGCTGTTGGTTTAA |
| sequence | | ANGPTL3-B6 | | | |
| of human | | | | | |
| ANGPTL | | | | | |
| 3 target | | | | | |
| site | | | | | |
| DNA | 6220 | MG29-1-human | Nucleotide | N.A. | ACTATGCTGTTGGTTTAATTGT |
| sequence | | ANGPTL3-C6 | | | |
| of human | | | | | |
| ANGPTL | | | | | |
| 3 target | | | | | |
| site | | | | | |
| DNA | 6221 | MG29-1-human | Nucleotide | N.A. | ATTGTTTATATTGGTCTTCCAC |
| sequence | | ANGPTL3-D6 | | | |
| of human | | | | | |
| ANGPTL | | | | | |
| 3 target | | | | | |
| site | | | | | |
| DNA | 6222 | MG29-1-human | Nucleotide | N.A. | TATTGGTCTTCCACGGTCTGGA |
| sequence | | ANGPTL3-E6 | | | |
| of human | | | | | |
| ANGPTL | | | | | |
| 3 target | | | | | |
| site | | | | | |
| DNA | 6223 | MG29-1-human | Nucleotide | N.A. | TATTCTTTTATCAGCTCAGAAG |
| sequence | | ANGPTL3-F6 | | | |
| of human | | | | | |
| ANGPTL | | | | | |
| 3 target | | | | | |
| site | | | | | |
| DNA | 6224 | MG29-1-human | Nucleotide | N.A. | ATCAGCTCAGAAGGACTAGTAT |
| sequence | | ANGPTL3-G6 | | | |
| of human | | | | | |
| ANGPTL | | | | | |
| 3 target | | | | | |
| site | | | | | |
| DNA | 6225 | MG29-1-human | Nucleotide | N.A. | TCAGCTCAGAAGGACTAGTATT |
| sequence | | ANGPTL3-H6 | | | |
| of human | | | | | |
| ANGPTL | | | | | |
| 3 target | | | | | |
| site | | | | | |
| DNA | 6226 | MG29-1-human | Nucleotide | N.A. | TCTATCTTCCAAGCCAAGAGCA |
| sequence | | ANGPTL3-A7 | | | |
| of human | | | | | |
| ANGPTL | | | | | |
| 3 target | | | | | |
| site | | | | | |
| DNA | 6227 | MG29-1-human | Nucleotide | N.A. | TGTGGGTTCTTGAATACTAGTC |
| sequence | | ANGPTL3-B7 | | | |
| of human | | | | | |
| ANGPTL | | | | | |
| 3 target | | | | | |
| site | | | | | |
| DNA | 6228 | MG29-1-human | Nucleotide | N.A. | ATTGATTCTAGGCATTCCTGCT |
| sequence | | ANGPTL3-C7 | | | |
| of human | | | | | |
| ANGPTL | | | | | |
| 3 target | | | | | |
| site | | | | | |
| DNA | 6229 | MG29-1-human | Nucleotide | N.A. | TTGATTCTAGGCATTCCTGCTG |
| sequence | | ANGPTL3-D7 | | | |
| of human | | | | | |
| ANGPTL | | | | | |
| 3 target | | | | | |
| site | | | | | |
| DNA | 6230 | MG29-1-human | Nucleotide | N.A. | TAACAGAGGTGAACATACAAGT |
| sequence | | ANGPTL3-E7 | | | |
| of human | | | | | |
| ANGPTL | | | | | |
| 3 target | | | | | |
| site | | | | | |
| DNA | 6231 | MG29-1-human | Nucleotide | N.A. | TCATGTCTACTGTGATGTTATA |
| sequence | | ANGPTL3-F7 | | | |
| of human | | | | | |
| ANGPTL | | | | | |
| 3 target | | | | | |
| site | | | | | |
| DNA | 6232 | MG29-1-human | Nucleotide | N.A. | CATGTCTACTGTGATGTTATAT |
| sequence | | ANGPTL3-G7 | | | |
| of human | | | | | |
| ANGPTL | | | | | |
| 3 target | | | | | |
| site | | | | | |
| DNA | 6233 | MG29-1-human | Nucleotide | N.A. | ATGTCTACTGTGATGTTATATC |
| sequence | | ANGPTL3-H7 | | | |
| of human | | | | | |
| ANGPTL | | | | | |
| 3 target | | | | | |
| site | | | | | |
| DNA | 6234 | MG29-1-human | Nucleotide | N.A. | ACCTGATATAACATCACAGTAG |
| sequence | | ANGPTL3-A8 | | | |
| of human | | | | | |
| ANGPTL | | | | | |
| 3 target | | | | | |
| site | | | | | |
| DNA | 6235 | MG29-1-human | Nucleotide | N.A. | CCTGATATAACATCACAGTAGA |
| sequence | | ANGPTL3-B8 | | | |
| of human | | | | | |
| ANGPTL | | | | | |
| 3 target | | | | | |
| site | | | | | |
| DNA | 6236 | MG29-1-human | Nucleotide | N.A. | ATTCATTATATTCAGGTAGTCC |
| sequence | | ANGPTL3-C8 | | | |
| of human | | | | | |
| ANGPTL | | | | | |
| 3 target | | | | | |
| site | | | | | |
| DNA | 6237 | MG29-1-human | Nucleotide | N.A. | TAGTTCTCCCACGTTTCATTGA |
| sequence | | ANGPTL3-D8 | | | |
| of human | | | | | |
| ANGPTL | | | | | |
| 3 target | | | | | |
| site | | | | | |
| DNA | 6238 | MG29-1-human | Nucleotide | N.A. | ATTGAAGTTTTGTGATCCATCT |
| sequence | | ANGPTL3-E8 | | | |
| of human | | | | | |
| ANGPTL | | | | | |
| 3 target | | | | | |
| site | | | | | |
| DNA | 6239 | MG29-1-human | Nucleotide | N.A. | GTGATCCATCTATTCGATGTTG |
| sequence | | ANGPTL3-F8 | | | |
| of human | | | | | |
| ANGPTL | | | | | |
| 3 target | | | | | |
| site | | | | | |
| DNA | 6240 | MG29-1-human | Nucleotide | N.A. | TGATCCATCTATTCGATGTTGA |
| sequence | | ANGPTL3-G8 | | | |
| of human | | | | | |
| ANGPTL | | | | | |
| 3 target | | | | | |
| site | | | | | |
| DNA | 6241 | MG29-1-human | Nucleotide | N.A. | CTTTTCAGGAGAATTTTGGTTG |
| sequence | | ANGPTL3-H8 | | | |
| of human | | | | | |
| ANGPTL | | | | | |
| 3 target | | | | | |
| site | | | | | |
| DNA | 6242 | MG29-1-human | Nucleotide | N.A. | TTTTCAGGAGAATTTTGGTTGG |
| sequence | | ANGPTL3-A9 | | | |
| of human | | | | | |
| ANGPTL | | | | | |
| 3 target | | | | | |
| site | | | | | |
| DNA | 6243 | MG29-1-human | Nucleotide | N.A. | CAGGAGAATTTTGGTTGGGCCT |
| sequence | | ANGPTL3-B9 | | | |
| of human | | | | | |
| ANGPTL | | | | | |
| 3 target | | | | | |
| site | | | | | |
| DNA | 6244 | MG29-1-human | Nucleotide | N.A. | AGGAGAATTTTGGTTGGGCCTA |
| sequence | | ANGPTL3-C9 | | | |
| of human | | | | | |
| ANGPTL | | | | | |
| 3 target | | | | | |
| site | | | | | |
| DNA | 6245 | MG29-1-human | Nucleotide | N.A. | GGTTGGGCCTAGAGAAGATATA |
| sequence | | ANGPTL3-D9 | | | |
| of human | | | | | |
| ANGPTL | | | | | |
| 3 target | | | | | |
| site | | | | | |
| DNA | 6246 | MG29-1-human | Nucleotide | N.A. | GTTGGGCCTAGAGAAGATATAC |
| sequence | | ANGPTL3-E9 | | | |
| of human | | | | | |
| ANGPTL | | | | | |
| 3 target | | | | | |
| site | | | | | |
| DNA | 6247 | MG29-1-human | Nucleotide | N.A. | ACGAATTGAGTTGGAAGACTGG |
| sequence | | ANGPTL3-F9 | | | |
| of human | | | | | |
| ANGPTL | | | | | |
| 3 target | | | | | |
| site | | | | | |
| DNA | 6248 | MG29-1-human | Nucleotide | N.A. | CGAATTGAGTTGGAAGACTGGA |
| sequence | | ANGPTL3-G9 | | | |
| of human | | | | | |
| ANGPTL | | | | | |
| 3 target | | | | | |
| site | | | | | |
| DNA | 6249 | MG29-1-human | Nucleotide | N.A. | TACTTGGGAAATCACGAAACCA |
| sequence | | ANGPTL3-H9 | | | |
| of human | | | | | |
| ANGPTL | | | | | |
| 3 target | | | | | |
| site | | | | | |
| DNA | 6250 | MG29-1-human | Nucleotide | N.A. | ACTTGGGAAATCACGAAACCAA |
| sequence | | ANGPTL3-A10 | | | |
| of human | | | | | |
| ANGPTL | | | | | |
| 3 target | | | | | |
| site | | | | | |
| DNA | 6251 | MG29-1-human | Nucleotide | N.A. | CTTGGGAAATCACGAAACCAAC |
| sequence | | ANGPTL3-B10 | | | |
| of human | | | | | |
| ANGPTL | | | | | |
| 3 target | | | | | |
| site | | | | | |
| DNA | 6252 | MG29-1-human | Nucleotide | N.A. | GTGTTTTCTACTTGGGATCACA |
| sequence | | ANGPTL3-C10 | | | |
| of human | | | | | |
| ANGPTL | | | | | |
| 3 target | | | | | |
| site | | | | | |
| DNA | 6253 | MG29-1-human | Nucleotide | N.A. | CTACTTGGGATCACAAAGCAAA |
| sequence | | ANGPTL3-D10 | | | |
| of human | | | | | |
| ANGPTL | | | | | |
| 3 target | | | | | |
| site | | | | | |
| DNA | 6254 | MG29-1-human | Nucleotide | N.A. | TACTTGGGATCACAAAGCAAAA |
| sequence | | ANGPTL3-E10 | | | |
| of human | | | | | |
| ANGPTL | | | | | |
| 3 target | | | | | |
| site | | | | | |
| DNA | 6255 | MG29-1-human | Nucleotide | N.A. | GCTTTGTGATCCCAAGTAGAAA |
| sequence | | ANGPTL3-F10 | | | |
| of human | | | | | |
| ANGPTL | | | | | |
| 3 target | | | | | |
| site | | | | | |
| DNA | 6256 | MG29-1-human | Nucleotide | N.A. | CTTTGTGATCCCAAGTAGAAAA |
| sequence | | ANGPTL3-G10 | | | |
| of human | | | | | |
| ANGPTL | | | | | |
| 3 target | | | | | |
| site | | | | | |
| DNA | 6257 | MG29-1-human | Nucleotide | N.A. | TGATCCCAAGTAGAAAACACCA |
| sequence | | ANGPTL3-H10 | | | |
| of human | | | | | |
| ANGPTL | | | | | |
| 3 target | | | | | |
| site | | | | | |
| DNA | 6258 | MG29-1-human | Nucleotide | N.A. | TTTTCCGGGATTGCATTGGGGA |
| sequence | | ANGPTL3-A11 | | | |
| of human | | | | | |
| ANGPTL | | | | | |
| 3 target | | | | | |
| site | | | | | |
| DNA | 6259 | MG29-1-human | Nucleotide | N.A. | CCGGGATTGCATTGGGGACATT |
| sequence | | ANGPTL3-B11 | | | |
| of human | | | | | |
| ANGPTL | | | | | |
| 3 target | | | | | |
| site | | | | | |
| DNA | 6260 | MG29-1-human | Nucleotide | N.A. | CGGGATTGCATTGGGGACATTG |
| sequence | | ANGPTL3-C11 | | | |
| of human | | | | | |
| ANGPTL | | | | | |
| 3 target | | | | | |
| site | | | | | |
| DNA | 6261 | MG29-1-human | Nucleotide | N.A. | GTGATTTCCCAAGTAAAAAGAA |
| sequence | | ANGPTL3-D11 | | | |
| of human | | | | | |
| ANGPTL | | | | | |
| 3 target | | | | | |
| site | | | | | |
| DNA | 6262 | MG29-1-human | Nucleotide | N.A. | TTGTCTTTCCAGTCTTCCAACT |
| sequence | | ANGPTL3-E11 | | | |
| of human | | | | | |
| ANGPTL | | | | | |
| 3 target | | | | | |
| site | | | | | |
| DNA | 6263 | MG29-1-human | Nucleotide | N.A. | CAGTCTTCCAACTCAATTCGTA |
| sequence | | ANGPTL3-F11 | | | |
| of human | | | | | |
| ANGPTL | | | | | |
| 3 target | | | | | |
| site | | | | | |
| DNA | 6264 | MG29-1-human | Nucleotide | N.A. | GGAGGCTGGTGGTGGCATGATG |
| sequence | | ANGPTL3-G11 | | | |
| of human | | | | | |
| ANGPTL | | | | | |
| 3 target | | | | | |
| site | | | | | |
| DNA | 6265 | MG29-1-human | Nucleotide | N.A. | AAATTTGCCTCAGTTCATTCAA |
| sequence | | ANGPTL3-H11 | | | |
| of human | | | | | |
| ANGPTL | | | | | |
| 3 target | | | | | |
| site | | | | | |
| DNA | 6266 | MG29-1-human | Nucleotide | N.A. | AATTTGCCTCAGTTCATTCAAA |
| sequence | | ANGPTL3-A12 | | | |
| of human | | | | | |
| ANGPTL | | | | | |
| 3 target | | | | | |
| site | | | | | |
| DNA | 6267 | MG29-1-human | Nucleotide | N.A. | CCTCAGTTCATTCAAAGCTTTC |
| sequence | | ANGPTL3-B12 | | | |
| of human | | | | | |
| ANGPTL | | | | | |
| 3 target | | | | | |
| site | | | | | |
| DNA | 6268 | MG29-1-human | Nucleotide | N.A. | TGAATCTGTTGGATGGATCAAC |
| sequence | | ANGPTL3-C12 | | | |
| of human | | | | | |
| ANGPTL | | | | | |
| 3 target | | | | | |
| site | | | | | |
| DNA | 6269 | MG29-1-human | Nucleotide | N.A. | TAGAGTATAACCTTCCATTTTG |
| sequence | | ANGPTL3-D12 | | | |
| of human | | | | | |
| ANGPTL | | | | | |
| 3 target | | | | | |
| site | | | | | |
| DNA | 6270 | MG29-1-human | Nucleotide | N.A. | GAGACTTCCAAGATAATCCTCT |
| sequence | | ANGPTL3-E12 | | | |
| of human | | | | | |
| ANGPTL | | | | | |
| 3 target | | | | | |
| site | | | | | |
| DNA | 6271 | MG29-1-human | Nucleotide | N.A. | AGACTTCCAAGATAATCCTCTT |
| sequence | | ANGPTL3-F12 | | | |
| of human | | | | | |
| ANGPTL | | | | | |
| 3 target | | | | | |
| site | | | | | |
| DNA | 6272 | MG29-1-human | Nucleotide | N.A. | GCTCTTGGTTTGTTATATTTAC |
| sequence | | ANGPTL3-G12 | | | |
| of human | | | | | |
| ANGPTL | | | | | |
| 3 target | | | | | |
| site | | | | | |
| DNA | 6273 | MG29-1-human | Nucleotide | N.A. | CTCTTGGTTTGTTATATTTACC |
| sequence | | ANGPTL3-H12 | | | |
| of human | | | | | |
| ANGPTL | | | | | |
| 3 target | | | | | |
| site | | | | | |
| MG91 | 6274 | MG91-666 | Protein | Unknown | MANNKNDNEGRFVYTFNCLKTNTAQEHILNEMFKQATSLYNDIQRQMLNTYKFIISHNAYKNAE |
| active | | effector | | | TRKEKNDFIKNFKIDVKSARRGIISKSFNGDKGYIASLSVRYGVNYPYISGTIAEDFGKNAWTAWE |
| effector | | | | | KKLWGNGKRITFHSNENPIKSISTRYKTSNKKLSGMDFDEDVKNVIITVGKHKLTIPIISRGTEYD |
| | | | | | AYAIDLIKNHMFSNGTIVRKKIRGKIKYELQITLKGVPYNKMRKLGKGNVGVDIGMSMVATYG |
| | | | | | NKLSLDALSAENKKEYQKELEVLERKMDRSRRATNPDNYDDKGRIVGKEKRTPWVYSKNYNVL |
| | | | | | KNKHSEIFRRYTNKRKILQNDLSNKLLEMGDTFYIENCNIAGMAKRAKETTVNPKTGRPRSKKR |
| | | | | | LGKSIQSNAPSEFLETLKRKVTTLGGTVYNVNPQIAATQFDFTDGTFKKHSLSERNVTLSNGNKH |
| | | | | | TRDGIAAFNLKHSKVGATEKLVDSYNITEMMNDYDKFIISERQEMNEHKNGLKISKNSMGIFGC* |
| MG91 | 6275 | MG91-667 | Protein | Unknown | MYCIKFPLKTKSSDEARIDKFFIHCCMVYNRVNGILSKEWRRITEECKDDNGKVDYKMRRKIAK |
| active | | effector | | | EISYNEGGISSDATKEGGKTYSMFSRFGITNIIACICKEDIGNGHSYANLGENNISISSKYQSQIALR |
| effector | | | | | LTGAWEKVMKENAVSHKKKCSEWGSMKSNINEKTKHILIDIEKKTLSVKYGKNNYMVIPFVAN |
| | | | | | PSKKEYEAVALSSTIKELGITSEVVRGRKRYYLTATVDGTPYNKGRKLGEGAIGIDPGVSSVTYY |
| | | | | | GKTVGQYKMDTDIKKLEDEKAKLLRYLDRSRRATNKDNYTADGQIKRGIKLNWVKSNRYIVAQ |
| | | | | | NRLREIERSISDKRKREQIDFVNEMLSEGNELHIEKNDVSSWSRRKSGITKGKNGRIKSNKRFGKS |
| | | | | | LHYAAPAQFVTIAKNKFTALGGTVVEVPSSVAAATATDHTSNFERTKRELKERSVKLSDGTVHD |
| | | | | | RDAHAAFNLKHCREDGKYDEEGMLSDYGNFCREEQKAWDQLKYQK* |
| MG91 | 6276 | MG91-668 | Protein | Unknown | MAKKENTNTNPSFVIEFRMHPEPWQADILSKQMEYLRHLYNRANSILLREYKKMIATPEFKEAV |
| active | | effector | | | ESKKKKNIAEVVKNYKFVVDMGVFVQEVTFSEFGFKGLVRRFGKLLIDDNSIYSDKGINTTMLGI |
| effector | | | | | VSNRLWSAYDKLLFDKKCTMTHFKSIGMFNSLPFGFSSGNLIGIQNIDFKNNVIVIRRKKQTGMSI |
| | | | | | RFEGIKTHYDNLALNGGDVTIKQLTIVRRTIRGKERYFVQFTINGTAPSTGRCIGTGTLGLDMGP |
| | | | | | HSLKYVADDAIDYINLSDSSNEDFAEYKRLQRKLDRSRRATNLHMYDEQGCSIKGAKQTVKSNR |
| | | | | | YKKTEAKLNDMKRQAAARRKIAQNIAANQVLVHGNHIVVEDNPFKGWQKRRSGKAFNKKGRQ |
| | | | | | LSKKRFGRSILKGAPSQFVTILENKANASGGWMYKASCKNAASKFDIFTKEVDNDIKLSDRRVT |
| | | | | | MSDGIEHDRDYNAAFKLKHLKPFSKEQKDYDYVAMYRDYPKFCEMEKTRESSLKQ* |
| MG91 | 6277 | MG91-669 | Protein | Unknown | MQFTVVIPFKPSNELKNDIDKGLKLLTTRYNTEAKKLKKRYDFWKNTKAYKIAEKEGIDSVSKF |
| effector | | effector | | | WEERSFNGEYGIDKLFKINNFKTHTKPKKNIYKNTIVNASMLQILRANLWRSVDKLIKGKGKNIR |
| | | | | | YVTMQTIPFKSLRGIKWNQKDSVFSFTHKRGHVITQHIDVKTPYELHAFNNCDMRMVTIKKEIIR |
| | | | | | GKEKYFFHICFEGTPYNKGRALGTASVGIDPSLENMYACFSNGDMEKLSLIQRIDEEFDIYHKVH |
| | | | | | VNKIDLLRQKLDRQRRANNPQFFNENGTINREALKQANYTWNDSNGYKKTRNALKDIQRKIAL |
| | | | | | RRKQCHFALANYILSKANKIIVEHNKFAAFAKKSQKDTYKPNGQQYSKRRFGRSINHGAPSYFIT |
| | | | | | ILKNKALCWGEKASFKLLSEFNGCTKFDHTNQEFNNEIKRNDKVVTLSNGDKVDRDLHAAMNIL |
| | | | | | FCENGIEQNARRRVEKSADHFNIAIMEEFYNQHKDKMIAI* |
| MG91 | 6278 | MG91-670 | Protein | Unknown | MPFKPSNELQHDIEQGFKLLTIRYNTELKKLKKRYDYLIKTQEYEIAKREKHEKTFWEDNNFTK |
| effector | | effector | | | YDFYEFFFRINSFKRAQHPQQSTYDNTIVSSTMLRALGDHLLSSFDKLLYQNGKDVHPKKISSMTF |
| | | | | | RGLVQIRYNSQTQSFEFTHRGRRNIISQKVDIKTDYEIYAFNNPIKEVTIKKQIIRGKEKYFFHICF |
| | | | | | RGTPYNKGRELGTNSVGIDPSMLKMFACYSNGEMIEHSLLDKIDEKSGEICDSKYNSDKYEKLE |
| | | | | | KKIGVLQTQINRRRRIENPKFFKENGEINEEALNQARLEHPNSDPWKYSKATIKMRNKVAQIKR |
| | | | | | NLALHRKECHFRLANYILSKANTIKVENNSYKSFQARAKETTYNANGRPRSKKRFGKSIQKGAPS |
| | | | | | AFITILKNKAASWGDRVSFSEVGASEACTQFDHTNDEFTKHELKERVVELSNGDRVHRDFHAAM |
| | | | | | NILFFKNTVTKSKKGTKKDSDHFDIEGLREFYSQHRENMIT* |
| MG91 | 6279 | MG91-671 | Protein | Unknown | MSDSSTEKSVFVVTFPLKTEKWQEDRINKMMRLLTVFYNEKQKVLLERWMHIRNSAEYKEHKE |
| active | | effector | | | EKSKNSLTAYMRNFGFSEFGFKEFFKDKNQTDSPYITHGLNSAILRNLAKSAWSAWSKKLEEKK |
| effector | | | | | KNIFIHTDKDVSIIKSDFKSSNNKISGFDVNYEDYSITMSASKPHVHTMFTIPFVVDRNSEYELFAL |
| | | | | | GEIQNDPTKLRNLAIVRKEVRGKYKYYVQFSIAGKPYNKGRQLGSGVVGIDPGPSKIAVVSDTAV |
| | | | | | RIIPLAKSIERDERETRRLQRKLDRSRRAMNPDNYNEDGTLSKGKHEWVKSHHYEETRSILADR |
| | | | | | QRKLAAKRKIAHNELANELLQMGNEFHVENNSFRSMQVRAKETTKNANGKNRTKKRFGKSLA |
| | | | | | NRAPSEFLIILENKVKQYENGVYVDIPDSIACTQYDFTSGDFSAHELRERTITTSDGIRHDRDALAA |
| | | | | | FNMKFVRTEQVVEKKKVKKAVDNFDNESMAEFYPLFCQMENKE* |
| MG91 | 6280 | MG91-672 | Protein | Unknown | MPNNIDKSVFVVTFPLKTEVWQEDRINKMMRLLTQLYNDKQDLLLRRYIHLSHSAEFKEARKT |
| active | | effector | | | GIKTFSKFMTEQGFSKFGIEKVFSDSSKSDSLQNQQLLCHGLNSDIIQELSHRSWSAWEKKLFGH |
| effector | | | | | GKFIKTNNEVDTLKSRAHKGNVTGFRCSMVDFTLTMTATKPNKHIVFTIPFVVDRNSEYELFALN |
| | | | | | QEIRNIAIVRKKIRGRYKYFVQFSFAGVPHNKGRKLGKGVVGIDPGPSKIAVVSDTEVKIMPLAES |
| | | | | | IREDEREKARIQRKLDRSRRAMNPDNYNEDGTISKGRHEWNKSNHYIALQSKLADNQRKLATKR |
| | | | | | KIAHNELANELLAMGNEFRVENNSFRSMQARTKDTTKNARGKNRSKKRFGKSLYNCAPSEFLTI |
| | | | | | LQNKVNQYEDGKYVDIPASTACTQYDFTNGQFTKHELRERTITTSDGKQHDRDALAAFNMKFV |
| | | | | | REEKVVGKKKIEKSDKNFDNESMATFYPRFCQMENKH* |
| MG91 | 6281 | MG91-673 | Protein | Unknown | MAHKENQKAFVVTFPLRTEKWQEDRIDKMMRMLTTFYNDRQRKLVRRYIYLSHSKAYKEAKG |
| active | | effector | | | KGVVAFKNYMKENGFSQYGLDAFFKADTKGALYQCGLNSMFLQYLSQCAWSAWDKKLFGKG |
| effector | | | | | DFVKTDKVVNIFCSRNKKGRFCGFDYDLSTFTIRIKSTCTKEIICSIPFVVNKNSEYELYALSQKIC |
| | | | | | RIGILRKLIRSKYKYYVQFTFDGVPYNKGRNIGTGIVGIDPGPSKIAIVGDNKVGIAKLAHGIEEDE |
| | | | | | RKTARLKRKLDRSRRATNPHMYKEDGTIIKGQRQTCFSKAYNETRKQLADAQRKLAAKRKIAH |
| | | | | | NELANTLLEYGDTFKVEANSYKSMQARAKATSMTKSGRIRSKKRYGKSIKNRAPSEFLVILKNK |
| | | | | | LLYYSKGKYYDVPSSYACTQFDFTNESFTEHKISERRIVTSDGIQHNRDTLAAFNIKNAIVNDTST |
| | | | | | KKKKVTKSKEFFDIVKMKASYNEFCVMEHYLLSE* |
| MG91 | 6282 | MG91-670 | Nucleotide | Unknown | GAAATAAAAAAATAGCCAATAGCTTGATATTATTATATCATGGGTTTGTACTACCAGGTAAG |
| intergenic | | intergenic | | | AAGCTATTTGGTACAATCTCAATGGCAACTATGCCATCTTACTAACGTCAAGGCGGTTTAAA |
| region | | region 2 | | | CCTAACTTTCAAGCGCCGTACAAAAAAAATATGGTAAAACATTTTATTTTTGTGTCTTGAATT |
| potentially | | | | | CGATTTTTTCTCGCTGATTGCGACCCTAAGGTGCTGAAATAGAAGAAGATACACGCTATA |
| encoding | | | | | |
| tracrRNA | | | | | |
| MG91 | 6283 | MG91-670 repeat | Nucleotide | Unknown | CTTGTACTTACCCTATATTTTAGGG |
| CRISPR | | | | | |
| repeat | | | | | |
| MG91 | 6284 | MG91-666 sgRNA1 | Nucleotide | Artificial | ACAGAAAAGGAUUUGUUAUCCUACAUCAUCGUUAAGAUGUUUAAUUAACUUUUAGUUCUUU |
| active | | | | sequence | GAAUAUUUGAUGAAAUAAUGUGAGUGGAACAUAAAUAAUAAAGUGUUAGGUAACAGAGAC |
| effector | | | | | ACCUUAAAAUUAUGGAAAAUUUCACUAUCCUUUCGAAAGAAGGGGUAAAACAGG |
| sgRNA | | | | | |
| MG91 | 6285 | MG91-666 sgRNA1 | Nucleotide | Artificial | ACAGAAAAGGAUUUGUUAUCCUACAUCAUCGUUAAGAUGUUUAAUUAACAUAUUGUUCUUU |
| active | | Mutant 1 | | sequence | GAAUAUUUGAUGAAAUAAUGUGAGUGGAACAUAAAUAAUAAAGUGUUAGGUAACAGAGAC |
| effector | | | | | ACCUUAAAAUUAUGGAAAAUUUCACUAUCCUUUCGAAAGAAGGGGUAAAACAGG |
| sgRNA | | | | | |
| MG91 | 6286 | MG91-666 sgRNA1 | Nucleotide | Artificial | ACAGAAAAGGAUUUGUUAUCCUACAUCAUCGUUAAGAUGUUUAAUUAACUAAUAGUUCUUU |
| active | | Mutant 2 | | sequence | GAAUAUUUGAUGAAAUAAUGUGAGUGGAACAUAAAUAAUAAAGUGUUAGGUAACAGAGAC |
| effector | | | | | ACCUUAAAAUUAUGGAAAAUUUCACUAUCCUUUCGAAAGAAGGGGUAAAACAGG |
| sgRNA | | | | | |
| MG91 | 6287 | MG91-667 sgRNA1 | Nucleotide | Artificial | AGGGUAAUGUGAAACCCAAGCGGAAGUUAUACGAAAAACAUAUUGUUCAAAAUAUGUCAGU |
| active | | | | sequence | AGUUUUCCGCAAAUCGUGCGUAAAAUAGAAUCUGGAUAGAUAAUGGUUUACCAUUGUUGAA |
| effector | | | | | AGCGGUUUCUUGCUGAAUUUAUUUCGGACACACGCAGCCUUUCGGAAAUGAAAGGUGAAC |
| sgRNA | | | | | ACAGGU |
| MG91 | 6288 | MG91-667 sgRNA1 | Nucleotide | Artificial | AGGGUAAUGUGAAACCCAAGCGGAAGUUAUACGAAAAACAUAUUGUUCAAAAUAUGUCAGU |
| active | | Mutant 1 | | sequence | AGUCUUCCGCAAAUCGUGCGUAAAAUAGAAUCUGGAUAGAUAAUGGUUUACCAUUGUUGAA |
| effector | | | | | AGCGGUUUCUUGCUGAAUUUAUUUCGGACACACGCAGCCUUUCGGAAAUGAAAGGUGAAC |
| sgRNA | | | | | ACAGGU |
| MG91 | 6289 | MG91-667 sgRNA1 | Nucleotide | Artificial | AGGGUAAUGUGAAACCCAAGCGGAAGUUAUACGAAAAACAUAUUGUUCAAAAUAUGUCAGU |
| active | | Mutant 2 | | sequence | AGACUUCCGCAAAUCGUGCGUAAAAUAGAAUCUGGAUAGAUAAUGGUUUACCAUUGUUGAA |
| effector | | | | | AGCGGUUUCUUGCUGAAUUUAUUUCGGACACACGCAGCCUUUCGGAAAUGAAAGGUGAAC |
| sgRNA | | | | | ACAGGU |
| MG91 | 6290 | MG91-668 sgRNA1 | Nucleotide | Artificial | CAGUGAUACAAAUGAAAUCCCGGUUUUGUUACGCCGGGUAUCAUCUGGACAAAUGUCCAG |
| active | | | | sequence | UUCAGCUGACGUUAAUUGCCGGGUUACUGGCAUAGUCAGCGAAAGUGCGAAACGGAAAAU |
| effector | | | | | AGAACCGUUAAACGACUUCUGCUUGCAGAGGUUCAAGUAGUCUAUGAGUACGUUAGCGUU |
| sgRNA | | | | | ACUUAUAUAGAAAUAUGUUGGUGAUUAACAAG |
| MG91 | 6291 | MG91-668 sgRNA1 | Nucleotide | Artificial | CAGUGAUACAAAUGAAAUCCCGGCGUUGUUACGCCGGGUAUCAUCUGGACAAAUGUCCAG |
| active | | Mutant 1 | | sequence | UUCAGCUGACGUUAAUUGCCGGGUUACUGGCAUAGUCAGCGAAAGUGCGAAACGGAAAAU |
| effector | | | | | AGAACCGUUAAACGACUUCUGCUUGCAGAGGUUCAAGUAGUCUAUGAGUACGUUAGCGUU |
| sgRNA | | | | | ACUUAUAUAGAAAUAUGUUGGUGAUUAACAAG |
| MG91 | 6292 | MG91-668 sgRNA1 | Nucleotide | Artificial | CAGUGAUACAAAUGAAAUCCCGGCUAUGUUACGCCGGGUAUCAUCUGGACAAAUGUCCAG |
| active | | Mutant 2 | | sequence | UUCAGCUGACGUUAAUUGCCGGGUUACUGGCAUAGUCAGCGAAAGUGCGAAACGGAAAAU |
| effector | | | | | AGAACCGUUAAACGACUUCUGCUUGCAGAGGUUCAAGUAGUCUAUGAGUACGUUAGCGUU |
| sgRNA | | | | | ACUUAUAUAGAAAUAUGUUGGUGAUUAACAAG |
| MG91 | 6293 | MG91-671 sgRNA1 | Nucleotide | Artificial | GGGAUUUGUAAUCCCGUUGUGAGCGAUAAUUGACACAAGGUGUCAUUGCUCAUCAAAAGA |
| active | | | | sequence | ACUAAUGGAACAAAAAGUGAACAAUAGGUCGUUCUCUGUACAAAAUCACAAAGAUGGAUGU |
| effector | | | | | CUCUGAAGGUACUAUCGAAAACUUUUAAGUACAUGGUUCAUGCCCUAUUUAUUGAUGAAAU |
| sgRNA | | | | | CAAUUUGUAGGGUAAAUACAAG |
| MG91 | 6294 | MG91-671 sgRNA1 | Nucleotide | Artificial | GGGAUUUGUAAUCCCGUUGUGAGCGAUAAUUGACACAAGGUGUCAUUGCUCAUCAAAAGA |
| active | | Mutant 1 | | sequence | ACUAAUGGAACUAUUAGUGAACAAUAGGUCGUUCUCUGUACAAAAUCACAAAGAUGGAUGU |
| effector | | | | | CUCUGAAGGUACUAUCGAAAACUAAUAAGUACAUGGUUCAUGCCCUAUUUAUUGAUGAAAU |
| sgRNA | | | | | CAAUUUGUAGGGUAAAUACAAG |
| MG91 | 6295 | MG91-671 sgRNA1 | Nucleotide | Artificial | GGGAUCUGUAAUCCCGUUGUGAGCGAUAAUUGACACAAGGUGUCAUUGCUCAUCAAAAGA |
| active | | Mutant 2 | | sequence | ACUAAUGGAACUAAUAGUGAACAAUAGGUCGUUCUCUGUACAAAAUCACAAAGAUGGAUGU |
| effector | | | | | CUCUGAAGGUACUAUCGAAAACUAUUAAGUACAUGGUUCAUGCCCUAUUUAUUGAUGAAAU |
| sgRNA | | | | | CAAUUUGUAGGGUAAAUACAAG |
| MG91 | 6296 | MG91-672 sgRNA1 | Nucleotide | Artificial | UGGAGAAUAAACAUUAAAGGAUUUGUAAUCCCGUUGUGAGCGCUAAUUGACACAGUGGUG |
| active | | | | sequence | UCAUUGCUCAUCGAAAGAGUGAUUGGAACAAAAGCGAACACCGUCUCGCUCUUGGCUUUUC |
| effector | | | | | UUUUUAAUUUUUGAGCUUUGAGAGUAAUGGUCGCAUGACACUUUUUAGUACAACAUUCUUA |
| sgRNA | | | | | CCUUACAUAGAAAUAUGUGGGGUAAAUACAAG |
| MG91 | 6297 | MG91-672 sgRNA1 | Nucleotide | Artificial | UGGAGAAUAAACAUUAAAGGAUUUGUAAUCCCGUUGUGAGCGCUAAUUGACACAGUGGUG |
| active | | Mutant 1 | | sequence | UCAUUGCUCAUCGAAAGAGUGAUUGGAACAAUAGCGAACACCGUCUCGCUCUUGGCUCAUC |
| effector | | | | | UAUAUAAUUGAUGAGCUUUGAGAGUAAUGGUCGCAUGACACAUAUUAGUACAACAUUCUUA |
| sgRNA | | | | | CCUUACAUAGAAAUAUGUGGGGUAAAUACAAG |
| MG91 | 6298 | MG91-672 sgRNA1 | Nucleotide | Artificial | UGGAGAAUAAACAUUAAAGGAUUUGUAAUCCCGUUGUGAGCGCUAAUUGACACAGUGGUG |
| active | | Mutant 2 | | sequence | UCAUUGCUCAUCGAAAGAGUGAUUGGAACAAUAGCGAACACCGUCUCGCUCUUGGCUUAUC |
| effector | | | | | UAUAUAUAUAAUGAGCUUUGAGAGUAAUGGUCGCAUGACACAUAUUAGUACAACAUUCUUA |
| sgRNA | | | | | CCUUACAUAGAAAUAUGUGGGGUAAAUACAAG |
| MG91 | 6299 | MG91-672 sgRNA2 | Nucleotide | Artificial | AUCUAUGGCAACGUUUUAUCCACGUUUUUGCCAAAUGGAGAAUAAACAUUAAAGGAUUUGU |
| active | | | | sequence | AAUCCCGUUGUGAGCGCUAAUUGACACAGUGGUGUCAUUGCUCAUCGAAAGAGUGAUUGG |
| effector | | | | | AACAAAAGCGAACACCGUCUCGCUCUUGGCUUUUCUUUUUAAUUUUUGAGCUUUGAGAGUA |
| sgRNA | | | | | AUGGUCGCAUGACACUUUUUAGUACAACAUUCUUACCUUACAUAGAAAUAUGUGGGGUAAA |
| | | | | | UACAAG |
| MG91 | 6300 | MG91-673 sgRNA1 | Nucleotide | Artificial | GAAAUAAUGGUAUUGUAAACCAACAUUAGCGCUAAUUGACAUAUAGUGUCAUUGCUAAAAU |
| active | | | | sequence | AAAGAUGUAUUGAAAGAAAGCAGAAUGAGAUCAUGUAGAAAACAUUCGUAAGAAUCUUUUC |
| effector | | | | | GAACGAUAUCAGAAACUCUUUAAAGUAAGUACAUCCUAUCCUUUGAAAGAAGGGUAGUUAC |
| sgRNA | | | | | AAG |
| MG91 | 6301 | MG91-673 sgRNA1 | Nucleotide | Artificial | GAAAUAAUGGUAUUGUAAACCAACAUUAGCGCUAAUUGACAUAUAGUGUCAUUGCUAAAAU |
| active | | Mutant 1 | | sequence | AAAGAUGUAUUGAAAGAAAGCAGAAUGAGAUCAUGUAGAUAUCAUUCGUAAGAAUCAUAUC |
| effector | | | | | GAACGAUAUCAGAAACUCUUUAAAGUAAGUACAUCCUAUCCUUUGAAAGAAGGGUAGUUAC |
| sgRNA | | | | | AAG |
| MG91 | 6302 | MG91-673 sgRNA1 | Nucleotide | Artificial | GAAAUAAUGGUAUUGUAAACCAACAUUAGCGCUAAUUGACAUAUAGUGUCAUUGCUAAAAU |
| active | | Mutant 2 | | sequence | AAAGAUGUAUUGAAAGAAAGCAGAAUGAGAUCAUGUAGAUAACAUUCGUAAGAAUCUUAUC |
| effector | | | | | GAACGAUAUCAGAAACUCUUUAAAGUAAGUACAUCCUAUCCUUUGAAAGAAGGGUAGUUAC |
| sgRNA | | | | | AAG |
| MG91 | 6303 | MG91-673 sgRNA2 | Nucleotide | Artificial | UGGAGCAUUAUCUCCUUUCAGAAUAAAGAAAUAAUGGUAUUGUAAACCAACAUUAGCGCUA |
| active | | | | sequence | AUUGACAUAUAGUGUCAUUGCUAAAAUAAAGAUGUAUUGAAAGAAAGCAGAAUGAGAUCAU |
| effector | | | | | GUAGAAAACAUUCGUAAGAAUCUUUUCGAACGAUAUCAGAAACUCUUUAAAGUAAGUACAU |
| sgRNA | | | | | CCUAUCCUUUGAAAGAAGGGUAGUUACAAG |
| MG91 | 6304 | MG91-155 sgRNA1 | Nucleotide | Artificial | AAAAGAAAGGCGUUGUUAGGCCUUGCGGACGUCAAGCUAAUGCCAGUGGGCGGCUGGUCC |
| active | | | | sequence | GACAAACGAUUUGCAGAAUGGUGGAAUGCAAAAAUAACAGUCAGUUCCUUCGAGAACGGG |
| effector | | | | | AGUAUAGAGCAAGUCCCCGAGUAUGCUUAUCAUAGUCUUUUUAUUGAAAAAUGUAGACUAU |
| sgRNA | | | | | UAACAGG |
| MG91 | 6305 | MG91-155 sgRNA1 | Nucleotide | Artificial | AAAAGAAAGGCGUUGUUAGGCCUUGCGGACGUCAAGCUAAUGCCAGUGGGCGGCUGGUCC |
| active | | Mutant 1 | | sequence | GACAAACGAUUUGCAGAAUGGUGGAAUGCAAAAAUAACAGUCAGUUCCUUCGAGAACGGG |
| effector | | | | | AGUAUAGAGCAAGUCCCCGAGUAUGCUUAUCAUAGUCUAUUCAUUGAAAAAUGUAGACUAU |
| sgRNA | | | | | UAACAGG |
| MG91 | 6306 | MG91-155 sgRNA1 | Nucleotide | Artificial | AAAAGAAAGGCGUUGUUAGGCCUUGCGGACGUCAAGCUAAUGCCAGUGGGCGGCUGGUCC |
| active | | Mutant 2 | | sequence | GACAAACGAUUUGCAGAAUGGUGGAAUGCAAAAAUAACAGUCAGUUCCUUCGAGAACGGG |
| effector | | | | | AGUAUAGAGCAAGUCCCCGAGUAUGCUUAUCAUAGUCUAUACAUUGAAAAAUGUAGACUAU |
| sgRNA | | | | | UAACAGG |
| MG91 | 6307 | MG91-201 sgRNA1 | Nucleotide | Artificial | CAUACAGAGGUUUUGUUAAGCCUCACAAUCUUAAUAAUUAAGUGUUCUUUGAAAUAUUUAG |
| active | | | | sequence | UUGAUUGUAAAUCUAUUUUGGGAAAUAAAAAAACAAAAAUUACAGUUAUUAGUUAACUAAG |
| effector | | | | | AAGAGUAUAGAGUUAGUUUUAAAGUACCAAAAUAUACCCUAAAUUAUUGGAAAUAAUAAAG |
| sgRNA | | | | | UUAGGGUACUAACAAGAA |
| MG91 | 6308 | MG91-201 sgRNA2 | Nucleotide | Artificial | CAUACAGAGGUUUUGUUAAGCCUCACAAUCUUAAUAAUUAAGUGUUCUUUGAAAUAUUUAG |
| active | | | | sequence | UUGAUUGUAAAUCUAUUUUGGGAAAUAAAAAAACAAAAAUUACAGUUAUUAGUUAACUAAG |
| effector | | | | | AAGAGUAUAGAGUUAGUUUUAAAGUACCAAAAUAUACCCUAAGAAAUUAGGGUACUAACAA |
| sgRNA | | | | | GAA |
| MG91 | 6309 | MG91-201 sgRNA2 | Nucleotide | Artificial | CAUACAGAGGCAUUGUUAUGCCUCACAAUCUUAAUAAUUAAGUGUUCUUUGAAAUAUUUAG |
| active | | Mutant 1 | | sequence | UUGAUUGUAAAUCUAUAUAGGGAAAUAAAAUAUCAAAAAUUACAGUUAUUAGUUAACUAAG |
| effector | | | | | AAGAGUAUAGAGUUAGAUAUAAAGUACCUAUAUAUACCCUAAGAAAUUAGGGUACUAACAA |
| sgRNA | | | | | GAA |
| MG91 | 6310 | MG91-201 sgRNA2 | Nucleotide | Artificial | CAUACAGAGGCAUCGUUAUGCCUCACAAUCUUAAUAAUUAAGUGUUCUUUGAAAUAUUUAG |
| active | | Mutant 2 | | sequence | UUGAUUGUAAAUCUAUAUUGGGAAAUAAAAUAUCAAAAAUUACAGUUAUUAGUUAACUAAG |
| effector | | | | | AAGAGUAUAGAGUUAGAUAUAAAGUACCAAUAUAUACCCUAAGAAAUUAGGGUACUAACAA |
| sgRNA | | | | | GAA |
| MG91 | 6311 | MG91-107 sgRNA1 | Nucleotide | Artificial | AGAAAUAUGGAUUUGUAAUCCAAUCUUAGCGUUAAUGGCAAACAAUUGCCAUUGCUAAAAC |
| active | | | | sequence | AAAGAUGUGAUGAAAUAAAGCAGAAUGUUAUCAUGUAAAAAUGGCUAUCUUGCCUUUUUGA |
| effector | | | | | ACGAUAGCAAAUACUAUUUAAAGUAAGAACAUCCUAUCCUAGAAAUAGGGUAGUUACAAG |
| sgRNA | | | | | |
| MG91 | 6312 | MG91-107 sgRNA1 | Nucleotide | Artificial | AGAAAUAUGGAUUUGUAAUCCAAUCUUAGCGUUAAUGGCAAACAAUUGCCAUUGCUAAAAC |
| active | | Mutant 1 | | sequence | AAAGAUGUGAUGAAAUAAAGCAGAAUGUUAUCAUGUAUAUAUGGCUAUCUUGCCUAUAUGA |
| effector | | | | | ACGAUAGCAAAUACUAUUUAAAGUAAGAACAUCCUAUCCUAGAAAUAGGGUAGUUACAAG |
| sgRNA | | | | | |
| MG91 | 6313 | MG91-107 sgRNA1 | Nucleotide | Artificial | AGAAAUAUGGAUUUGUAAUCCAAUCUUAGCGUUAAUGGCAAACAAUUGCCAUUGCUAAAAC |
| active | | Mutant 2 | | sequence | AAAGAUGUGAUGAAAUAAAGCAGAAUGUUAUCAUGUAUUAAUGGCUAUCUUGCCUUAAUGA |
| effector | | | | | ACGAUAGCAAAUACUAUUUAAAGUAAGAACAUCCUAUCCUAGAAAUAGGGUAGUUACAAG |
| sgRNA | | | | | |
| MG91 | 6314 | MG91-107 sgRNA2 | Nucleotide | Artificial | AGAAAUAUGGAUUUGUAAUCCAAUCUUAGCGUUAAUGGCAAACAAUUGCCAUUGCUAAAAC |
| active | | | | sequence | AAAGAUGUGAUGAAAUAAAGCAGAAUGUUAUCAUGUAAAAAUGGCUAUCUUGCCUUUUUGA |
| effector | | | | | ACGAUAGCAAAUACUAUUUAAAGUAAGAACAUCCUAUCCUAACUUUAUUGAAAAAUAUAGG |
| sgRNA | | | | | GUAGUUACAAG |
| MG91 | 6315 | MG91-69 sgRNA2 | Nucleotide | Artificial | UAAAAAAAGAUAUUAGGUUUUGUUAAGCCUAACAAUCGUUAAGUGUUCUUUGGAAUAUUGA |
| active | | | | sequence | UUGUAAAUCUAUUUUGGGAAAUAAAAAAGCAAAAAUUACAGUUAUCAGUUUACUGAGAAGA |
| effector | | | | | GUAUAGAGUUAGUUUUAAAGUACCAAAAUAUACCCUAAAUUAUUGAAAAAUAAAGUUAGGG |
| sgRNA | | | | | UACUAACAAG |
| MG91 | 6316 | MG91-69 sgRNA3 | Nucleotide | Artificial | CACGCAGUUGUACUGUAGGAUAUUAAUAAAAAAAGAUAUUAGGUUUUGUUAAGCCUAACAA |
| active | | | | sequence | UCGUUAAGUGUUCUUUGGAAUAUUGAUUGUAAAUCUAUUUUGGGAAAUAAAAAAGCAAAAA |
| effector | | | | | UUACAGUUAUCAGUUUACUGAGAAGAGUAUAGAGUUAGUUUUAAAGUACCAAAAUAUACCC |
| sgRNA | | | | | UAAAUUAUUGAAAAAUAAAGUUAGGGUACUAACAAG |
| MG91 | 6317 | MG91-69 sgRNA3 | Nucleotide | Artificial | CACGCAGUUGUACUGUAGGAUAUUAAUAAAAAAAGAUAUUAGGCAUUGUUAUGCCUAACAA |
| active | | Mutant 1 | | sequence | UCGUUAAGUGUUCUUUGGAAUAUUGAUUGUAAAUCUAUAUAGGGAAAUAAAAUAGCAAAAA |
| effector | | | | | UUACAGUUAUCAGUUUACUGAGAAGAGUAUAGAGUUAGCUAUAAAGUACCUAUAUAUACCC |
| sgRNA | | | | | UAAAUUAUUGAAAAAUAAAGUUAGGGUACUAACAAG |
| MG91 | 6318 | MG91-69 sgRNA3 | Nucleotide | Artificial | CACGCAGUUGUACUGUAGGAUAUUAAUAAAAAAAGAUAUUAGGCAUAGUAAUGCCUAACAA |
| active | | Mutant 2 | | sequence | UCGUUAAGUGUUCUUUGGAAUAUUGAUUGUAAAUCUAUAUAGGGAAAUAAAAUAGCAAAAA |
| effector | | | | | UUACAGUUAUCAGUUUACUGAGAAGAGUAUAGAGUUAGCUAUAAAGUACCUAUAUAUACCC |
| sgRNA | | | | | UAAAUUAUUGAAAAAUAAAGUUAGGGUACUAACAAG |
| MG91 | 6319 | MG91-2 sgRNA1 | Nucleotide | Artificial | AUAAAAAUAACAUACAGAGGUUUUGUUAAGCCUCACAAUCUUAAUAAAUAAGUGUUCUUUG |
| active | | | | sequence | AAAAUAUUUAGUUGAUUGUAAAUCUAUUUUGGGAAAUAAAAAAACAAAAAUUACAGUUAUU |
| effector | | | | | AGUUAACUAAGAAGAGUAUAGAGUUAGUUUUAAAGUACCAAAAUAUACCCUAAGAAAUUAG |
| sgRNA | | | | | GGUACUAACAAG |
| MG91 | 6320 | MG91-2 sgRNA1 | Nucleotide | Artificial | AUAAAAAUAACAUACAGAGGCUUUGUUAAGCCUCACAAUCUUAAUAAAUAAGUGUUCUUUG |
| active | | Mutant 1 | | sequence | AAAAUAUUUAGUUGAUUGUAAAUCUAUAUAGGGAAAUAAAAAAUCAAAAAUUACAGUUAUU |
| effector | | | | | AGUUAACUAAGAAGAGUAUAGAGUUAGAUUUAAAGUACCUAUAUAUACCCUAAGAAAUUAG |
| sgRNA | | | | | GGUACUAACAAG |
| MG91 | 6321 | MG91-2 sgRNA1 | Nucleotide | Artificial | AUAAAAAUAACAUACAGAGGCAUUGUUAUGCCUCACAAUCUUAAUAAAUAAGUGUUCUUUG |
| active | | Mutant 2 | | sequence | AAAAUAUUUAGUUGAUUGUAAAUCUAUAUAGGGAAAUAAAAAUUCAAAAAUUACAGUUAUU |
| effector | | | | | AGUUAACUAAGAAGAGUAUAGAGUUAGAAUUAAAGUACCUAUAUAUACCCUAAGAAAUUAG |
| sgRNA | | | | | GGUACUAACAAG |
| MG91 | 6322 | MG91-10 sgRNA3 | Nucleotide | Artificial | UAUAUUUGCGACAUAAAGGUGUUGUCAGGCCUUGCGUACGUUAAGCUAAUGCCAGUGGGC |
| active | | | | sequence | GGCUGGUGCGACAAACGAUUAUGCAGAAUGGUAGAAUGCAAAAAUAACAGUCAGUUCCUU |
| effector | | | | | CGGGAAUGGGAGUAUAGAGCAGGUCUCCGAGUAUGCAAAUCAUGGCCUUUUUAUUUCGAA |
| sgRNA | | | | | AGAAAUGUAGGCUAUUGACAGG |
| MG91 | 6323 | MG91-10 sgRNA4 | Nucleotide | Artificial | GACAUAAAGGUGUUGUCAGGCCUUGCGUACGUUAAGCUAAUGCCAGUGGGCGGCUGGUGC |
| active | | | | sequence | GACAAACGAUUAUGCAGAAUGGUAGAAUGCAAAAAUAACAGUCAGUUCCUUCGGGAAUGG |
| effector | | | | | GAGUAUAGAGCAGGUCUCCGAGUAUGCAAAUCAUGGCCUUUUUAUUUCGAAAGAAAUGUA |
| sgRNA | | | | | GGCUAUUGACAGG |
| MG91 | 6324 | MG91-10 sgRNA3 | Nucleotide | Artificial | GACAUAAAGGUGUUGUCAGGCCUUGCGUACGUUAAGCUAAUGCCAGUGGGCGGCUGGUGC |
| active | | Mutant 1 | | sequence | GACAAACGAUUAUGCAGAAUGGUAGAAUGCAAAAAUAACAGUCAGUUCCUUCGGGAAUGG |
| effector | | | | | GAGUAUAGAGCAGGUCUCCGAGUAUGCAAAUCAUGGCCUAUUCAUUUCGAAAGAAAUGUA |
| sgRNA | | | | | GGCUAUUGACAGG |
| MG91 | 6325 | MG91-10 sgRNA3 | Nucleotide | Artificial | GACAUAAAGGUGUUGUCAGGCCUUGCGUACGUUAAGCUAAUGCCAGUGGGCGGCUGGUGC |
| active | | Mutant 2 | | sequence | GACAAACGAUUAUGCAGAAUGGUAGAAUGCAAAAAUAACAGUCAGUUCCUUCGGGAAUGG |
| effector | | | | | GAGUAUAGAGCAGGUCUCCGAGUAUGCAAAUCAUGGCCUUAACAUUUCGAAAGAAAUGUA |
| sgRNA | | | | | GGCUAUUGACAGG |
| MG91 | 6326 | MG91-2 PAM (5′) | Nucleotide | Artificial | tnTYn |
| active | | | | sequence | |
| effector | | | | | |
| PAM (5′) | | | | | |
| MG91 | 6327 | MG91-10 PAM (5′) | Nucleotide | Artificial | GnGYCn |
| active | | sgRNA3 | | sequence | |
| effector | | | | | |
| PAM (5′) | | | | | |
| MG91 | 6328 | MG91-69 PAM (5′) | Nucleotide | Artificial | TTTY |
| active | | sgRNA3 | | sequence | |
| effector | | | | | |
| PAM (5′) | | | | | |
| MG91 | 6329 | MG91-107 PAM (5′) | Nucleotide | Artificial | Cc |
| active | | sgRNA1 | | sequence | |
| effector | | | | | |
| PAM (5′) | | | | | |
| MG91 | 6330 | MG91-155 PAM (5′) | Nucleotide | Artificial | Gnkynn |
| active | | sgRNA1 | | sequence | |
| effector | | | | | |
| PAM (5′) | | | | | |
| MG91 | 6331 | MG91-201 PAM (5′) | Nucleotide | Artificial | ttTYnAA |
| active | | sgRNA2 | | sequence | |
| effector | | | | | |
| PAM (5′) | | | | | |
| MG91 | 6332 | MG91-666 PAM (5′) | Nucleotide | Artificial | nnnCn |
| active | | sgRNA1 | | sequence | |
| effector | | | | | |
| PAM (5′) | | | | | |
| MG91 | 6333 | MG91-667 PAM (5′) | Nucleotide | Artificial | yYt |
| active | | sgRNA1 | | sequence | |
| effector | | | | | |
| PAM (5′) | | | | | |
| MG91 | 6334 | MG91-667 PAM (5′) | Nucleotide | Artificial | yYy |
| active | | sgRNA1 | | sequence | |
| effector | | | | | |
| PAM (5′) | | | | | |
| MG91 | 6335 | MG91-668 PAM (5′) | Nucleotide | Artificial | TtGc |
| active | | sgRNA1 | | sequence | |
| effector | | | | | |
| PAM (5′) | | | | | |
| MG91 | 6336 | MG91-668 PAM (5′) | Nucleotide | Artificial | tngn |
| active | | sgRNA1 | | sequence | |
| effector | | | | | |
| PAM (5′) | | | | | |
| MG91 | 6337 | MG91-671 PAM (5′) | Nucleotide | Artificial | gnGY |
| active | | sgRNA1 | | sequence | |
| effector | | | | | |
| PAM (5′) | | | | | |
| MG91 | 6338 | MG91-672 PAM (5′) | Nucleotide | Artificial | mCm |
| active | | sgRNA1 | | sequence | |
| effector | | | | | |
| PAM (5′) | | | | | |
| MG91 | 6339 | MG91-673 PAM (5′) | Nucleotide | Artificial | ryCC |
| active | | sgRNA1 | | sequence | |
| effector | | | | | |
| PAM (5′) | | | | | |
| MG29 | 6340 | MG29-15 effector | Protein | Unknown | GRTAEEGKKKIQGLNEYINLYNQKQEKNKRLPKLKLLYKQILSDRISTSFMAESFSEDQEVIDAIE |
| effector | | | | | EYYKFHLLAFQAEDKDDTENILEKVKELLSNIKEYDLSKIYLRNDTKITAISQKIFGNYGVFNTAL |
| | | | | | EYYYATAVKPDFQKEYEKANQKKRDTLDKAQTQFVKQPYVSIELLQTAIDAYIATIDKGEEIYKR |
| | | | | | YSPTCIADYFKNNFKAEKKEKNDKEYGFIDNIKAKYSCIQGILGTPYPKDKKLIQQKNDIPNIKAF |
| | | | | | LDSLMELLHFTQPLSIVDEREVTKILTSDRLKIDKEWAEEANKSFFEKDKKFYEQFDLYFKELQK |
| | | | | | LIPLYNKVRNYATKKPYSTEKFKLNFENKGQFLGGWVDSHTENSDNATQAGGYLFRKKNQIGE |
| | | | | | YDYFLGVSSDSKLFRSHLRNEIVDEDKSEFERLDYYQLKSASVYGNSYIGNQSYDKDKENLFDSII |
| | | | | | EFANRNNPAAKEDFNKYISSQKGDNKPTPNGLLKILQEKHSKALEELMIDGDFIRINTIVTDNLKN |
| | | | | | TILSLNRIPKSQEYKNTIFTLFTEPIQVIEELSKEKSFSYFPVSAKELEDSLNRELKPLLLFKISNQDL |
| | | | | | SYADSFSQGKRKSRGRENMHTLYFRQLMSGSQNILDIGTGEVFFRKSSIENPTIHKANEAVINKN |
| | | | | | PLAKKKDSAFEYDIIKDRRFTVDKFQFHLSIIMNYQKPQKASDFNFEVLEFLQNNPNVNIIGLDRG |
| | | | | | ERHLIYLTLINQKGEILLQESLNNISSENYPITTPYHDLLATKEKERDEARKSWGTIENIKELKEG |
| | | | | | YISQVVHKIAKLMVEHNAIVVMEDLNFGFKRGRFKVEKQVYQKLEKMLIDKLNYLVFKDKDPN |
| | | | | | EIGGLYKGLQLANKFESFQKMGKQSGFLFYVPAWNTSKIDPTTGFVNLFDTRYENLDKAKAFFS |
| | | | | | KFEDIRYNRTEKYFEFVVENYADFNAKAEGTKQDWTICSFGERIKNFRNPDALNQWDNKILNLT |
| | | | | | EEFKTLFTHNKIDITTELKEQIAQQNEAKFFKSLLELFKLTIQMRNSITNSEVDYLISPVKNKQGQ |
| | | | | | FFDSRKADDTQPKDADANGAYNVAQKGLMWLEQINKFDGKDWKKLDLDKTNKGWLQFIQNK |
| | | | | | TQ |
| MG29 | 6341 | MG29-16 effector | Protein | Unknown | MFLGFINKYQLSKTLRFELKPIGKTLEFIEQKGLIVEDEARAEDYKRVKELIDKYHKEFIHQALS |
| effector | | | | | GIRLDGLDRYEELFFIQNRDEKTQKEFEKLQDDLRKQIVAGFKAHEAFKNIDKKELITVELPKFL |
| | | | | | KDEKDKKDREIVKKFDSFTTYFTGFHENRKNIYSDKAQHSSIGYRVIHENLSIFLSNKRAFESIQQ |
| | | | | | NFPEIAQTAQNSLLEHLEGGVVEDMFGLDYFSRTLTQTYIDIYNTMLGGKVLKDGTKIQGLNEHI |
| | | | | | NLYRQKYNIEKRKLPNLKALHKQILSDRESMSWLHESFANRDELNSTVEKFYKESIISFKQYNDA |
| | | | | | VDITEELINILSDESDYDLGKVFVKNDISLTAISQEIFKDYRVIKDALWQKHLADNPKATKSKDITV |
| | | | | | DEEKYFSRKNSYFSISQIEKALNKAELSDEAKKEKENYKGLFDFFKTKVAESSKAVKENFTDWQN |
| | | | | | NKEDKKLTKSLLDSMLNLQRAIKPLSVKAEDADDRSFYALFSTYFESLSGVIRIYDKVRNFESKKP |
| | | | | | YSMEKFKLNFENKGNFLGGWVDSSTEKSDNGTQSGGYLFRKRNGIGEFDYYLGISSDPKLFRSH |
| | | | | | LQDEIEKEDISDFERLDYYQLKSATVFGNSYVGDSYSKDRDLLYQKILEFVENTEPLKADIDKYVS |
| | | | | | SQKGTNQPTPSGIISIVKEKYPELLQKLKDDQGFSEINKTVTDRLKKTILSLHRIPRSQEYKNHNFS |
| | | | | | LFTEAIEVIEELSSEKSFAYFKVSKSELENALNRDSKPLFLFKITNKDLSFADSFIAGKRKSRGTDN |
| | | | | | LHTLYFKALMSGSQNVFDIGTGEVFYRKEDYKGKKIVHKANEPIENKNRLNDKKHSLFEYDIVK |
| | | | | | NRRYLVDKFQFHLSIVQNYIKPKKYPDFNTEVNQAIKGASDIKVIGVDRGERHLLYLSLIDSSGRI |
| | | | | | VEQYSLNQIINSHNGKKHIVDYHQKLADKEKERAEARENWGVVENIKELKEGYMSHVIHRIATL |
| | | | | | MVKHNAIVALEDLNFGFKRGRFKVEKQVYQKFEKMLIDKLNYLVDKQKSPNELGGLLKAFQLT |
| | | | | | NKFVSFEKLGKQSGFLFYVPAWNTSKIDPVTGFVNLLDTRYQSIEKSKEFFSKFDAIRYNDQKGY |
| | | | | | FEFEFDYKNFTTKADGTRTKWTLCTYGTRIKTFRNRDKNHQWDNVEVDLTAEFKSLFGLHSGD |
| | | | | | LKELIISQDRKEFFETLLYLLRLTLQMRNSVTNSEIDYLISPVADKNGNFYDSRVASDDLPRDADA |
| | | | | | NGAYNIARKGLMIIEKIAKSKSGEKLNLTISNKEWLAYAQR |
| MG29 | 6342 | MG29-17 effector | Protein | Unknown | MFKNFTNQYQLSKTLRFELKPIGKTLNNINSKMLLEQDEKRASEYKVVKKIIDEYHKDFMNEAM |
| effector | | | | | DGFLFDNDDINSYERLFYIKDKSDEDKKAIESVQQKMRKKIAEKFQKHPLYKNLFAKELIKQDLL |
| | | | | | SWLDKKDNKFVTKIREITSDNTLMIDNMKTIVSNFSNFTTYFSGFHENRKNIYKADEKHTAVAYR |
| | | | | | VVHENLPIYLTNKRAFEKLNEKYPKLVSDAKNTVEHHLLGAVVEDMFTNDYFNHTASQTHIDLY |
| | | | | | NTMIGGTVLDDGTKVQGLNEKINLYRQQNGLTKKDIPNLKPLYKQILSDRELTSWLPESFESDNE |
| | | | | | LLNAINDFYNNEIVNFNCCDGVINILDKLKEHFADTLLFDKERIYIRNDLSMTNISKDIFGDWSVIK |
| | | | | | GAIEEKYKLDNPREVKAKDGLEKIEKKIEKTKYFSIAHIEEALRSYLRDKDELRAKMSSDVIFDYF |
| | | | | | NNWDKTTVEHTVSNNIFDRIIDSFIAIKPLLSSEYSLDKKLSNDQDSKSKIKNFMDAITNLQRFVKP |
| | | | | | LHAKIDSELDIAFYANFDTYFEQLSLGVKLYDMVRNYLTKKPFSTDKFKLNFESGYLLNGWSQD |
| | | | | | YDTRAGLLFEKDGNYYLGVNVKKLTEDEKQYLLDNPNENLSKRVILDFQKPDNKNIPRLFIRSK |
| | | | | | GDNFAPAVTIYNLPIQSVIEIYDNGKFKTEYRKKNPKDYLDSLHKLIDYFKQGFLQHESYKHYKF |
| | | | | | GWKNTKDYLDIAQFYKDVETSCYEVKYEEVNWDRLMNYVESGKLYLFQIYNKDFSPYSKGTPN |
| | | | | | MHTLYWKMLFDKDNLSNVVYKLNGQAEIFFRKKSITNNIVTHDANEPINNKNSDNPKKSSTFDY |
| | | | | | DIIKDKRYTVDKFGFHVPITMNFKANGNDNINQQVNQAIKESNDIKVIGIDRGERHLLYLSLIDSS |
| | | | | | GKIIEQYSLNEIVNEHNCTTYKTNYHKLLDAKEKERGDARVNWGVVENIKELKEGYMSQVIHK |
| | | | | | VATLMVKHNAIVVLEDLNFGFKQGRFKVEKQVYQKFEKALIDKLNYFVDKKKDVNKLGGVLN |
| | | | | | ALQLTSKFISFEKIGKQSGFLYYVPAWNTSKIDPITGFVNLFDTRYTSVEKAKEFFGKFESIKYNIS |
| | | | | | KNYFEFKLINYTAFNPKADGTRQNWTVCSYGERILIFRNKDKNSQWDNKEVKPTDGFKKLFDD |
| | | | | | NEIVYMNGQNIKDFILVKDEKAFFEVLLGCFKLTLQMRNSVTNSDIDYLISPVCDKNGIFYDSRAV |
| | | | | | EDSLPKDADANGAYNIARKGLWVIEQIKKVDDLRKIKLAISNKEWLSYVQTNDNYK |
| MG29 | 6343 | MG29-18 effector | Protein | Unknown | MAENKAIFDGFANKYSLQKTLRFGLIPDSESKKWIEKNLVIEKDEKLAEEYKKAKKIIDKVHKAF |
| effector | | | | | IESALEKLKLNEKALTAFEKETAKTKKERDKKTIEKIQASLRNEVADSFNKDKEEFKAMFSEKMI |
| | | | | | KENAPRFCNTAEEKATMKLFDNFTTYFKEFHKNRKNIYSNEAKATSIAFRIVHENLVTFVDNLRI |
| | | | | | FAKIREGGLDLDKAEYELKSILGKEKIEKLFSIEYFNKVLSQSGIDFYNRIIGGEFEEGSRKKIRGL |
| | | | | | NELINLHNQKERGKLPRFKQLKKQILSDRKKSLDFGFLDDSELLQAIEEFYLKELSDENGKRTPE |
| | | | | | MLRALFERAEEFDIEKIHLRNDSTLRELSNKLFGDWSAIENALSEHYEKENPSKDTKKYEKDKEK |
| | | | | | WLKQDQFPIATIETALSLYEHEKVDKGKCKGLFFRQFSSFKKEEDDKENLLERLEISYNRAKPIL |
| | | | | | ENKSPGNRLASDEQAKGKLKALLDALIDILHFVKSLRLKDAAISDKDYSFYGEFDPLFERLDGIV |
| | | | | | GIYNKTRNYLTKKPYSTEKIKLNFENSVLLGGWDRNIEDTKGGVIFRKDGQFYLGIINKNNKTIL |
| | | | | | KNPPKAKDGEVAFEKMFYKLIPNPARDLHHTILSKKRSAKYKPTKTLLEKYEQKKHIKGDNFDK |
| | | | | | KFCHELIDFFKESIKKNPDWETFNFKFSETSSYEDISGFYREVGRQGYKVVFERIPASYIETLVKE |
| | | | | | GKLYLFKIWSKDFSKDSKGTPNMHTLYWRALFDEKNLKEPIYKLNGEAEMFFRKRSVEPKITHP |
| | | | | | AGKPIPNKNPDNLKKESVFKYDLIKDRRYSLDKFQFHVPITTNFGSEGQEFIDYDVRDAIKKSPVR |
| | | | | | IIGIDRGERNLLYLSMIDENGKILLQESLNKITSLYGGGKKTTDYNSLLARSEAGRDEARRDWKK |
| | | | | | IENIKEIKEGYLSQAVHKIATLMVENEAIVVMEDLNSGFKRGRTKVEKQVYQKFERMLIEKLNY |
| | | | | | LVFKKRSPEEAGGLRNALQLTSKFKSFEKLGKQSGFLFYIPAAMTSKIDPVTGFANELNPKYESV |
| | | | | | GKSKEFFSKFERISYNSKKNWFEFSFDYSNFKTKDGLEGKWVVCSTPHERFYRNGGAKGGAKG |
| | | | | | ETLPMNANEELKKLFGEFGIEYSTGDCLKKEIVSKDSKEFFKKLTRVLASILSLRQNNGKTGQDE |
| | | | | | QDYILSPVEPFFCSLDGKDGLPKDADANGAYNIARKGLLAVRQIRSAEDPKKARLAIRNKEWLEF |
| | | | | | AQAMK |
| MG29 | 6344 | MG29-20 effector | Protein | Unknown | MQQMNTENIWGGLTNQYSLSKTLRFELKPVGFDGEELVIDESLNSIEKIIEEDKQRNEDFKIVKKI |
| effector | | | | | ADEYFKEFIERSLKFTKISKTKLEEFENIYLEFIRDKQNKNKRDNFDKLNKELRKNLKELVKLKF |
| | | | | | KDEFSTFFKKEFYEKVLPLWLDKRNRIEDKKLVEKFKGFTTYFTGFNSNRENVFSENNIPTSIFYR |
| | | | | | IVDDNLPKYLANYDKFKKILEVSNDKFGELQVELKDELEGLTLLEFLSIQNYNLFLNQSGIEKYN |
| | | | | | KRIIGKINSRINELIQRDKDLTREQIKILKKSKLQVLFNQILSDKESLFTFDKENSDYEVLTKIDEFY |
| | | | | | NNLNSKNQFDEFSNLFENLDSENFDLSLIKIKNGKFISDISQNIFGDYNVIKEDLKREYIKSRNYDIE |
| | | | | | KLNKKQQEEIEKYLKTDYFCFKEIQNSISRIKETRDDSDEHITLFDFFKDFKFSVEHKSIDLLKNIED |
| | | | | | SYKSFNSIKFEEFKNESQKKLTQDKYNEVVNVIKEFLDALQNYYHFVKLLKFENEYRDEHFYQIY |
| | | | | | DELLNLISQITPLYNKVRNYISQKPFSTQKFKLNFQSSSFLNGWDSNFETKSAVILKKKINNKTNY |
| | | | | | YIAISPKKIENNENDFSVNKGNFEILNYDFQKPDNKNIPRLFIRSKGDNFSPNVEKYNLPVQEIIEIY |
| | | | | | DKGYFKTEFRKTNFSKFKESLVKLIDYFKLGFQRHESYKHFKFNWKESREYEDISQFYFDVESSC |
| | | | | | YQLNFKKINEEYIKQLVEENKLYLFQIYNKDFSQNKIKKENYNSKKNLHTMYFEELFSEENLEDV |
| | | | | | VFKLNGQAEIFYREKSTEYKPTHPKNLPTKNKDPINGKEESLFSYDIGKNKRYTQDKVLFHVPITL |
| | | | | | NFKSNSRVRINNEVNKVIKQNSKNINILSLDRGERHLLYYTLLDTKGNIKEKGSFNLINDSFNRKV |
| | | | | | NYHQKLSKLEKERDEKRKSWQNISTIKELKEGFLSQIIHKISKIAVENNAIIVLEDLNYGFKRGRF |
| | | | | | KVEKQVYEKFEKMLISKLNFLVFKDKKNDEIGGNLKAYQLTPEVNVLKDIGKQTGILFYIDPYLT |
| | | | | | SKICPKTAFVNRLYPKYENESQAKDFINKFDSIRYLKDEDLFEFSFKYSSFGVKDLVKDDWKIYSN |
| | | | | | GIKLVQSRDKNQNNNWTTKSVNVNEELKKLFNDFNIKIEESQNLIDDIVKQNKFFLENIIKNLKLI |
| | | | | | LQLRNSYTDNELKINKITEKEGDYILSCVKNKKGYFFDSRNANKEEVDNADCNGAYHIGLKGLM |
| | | | | | VLNKIKEFEDIEKIKFNDLKIERNEFLNEMIRRNWS |
| MG29 | 6345 | MG29-21 effector | Protein | Unknown | MSTLATQFTGKFPLSKTIRFELIPRFGTKELLNGLFDSDYKRAELAPTVKEILNMYYQDFINICLE |
| effector | | | | | DANLQNVKIKGISALDYAFDAYKNNDSKKVTTANTALMTYISKCFTDKNKFGLDEYLNLLKLEK |
| | | | | | KKSPTILVKWINEKVNKGIFSKEKAAEYKNAISFFDKFITYFSGFKKTKENMFKPEDKASSIAHRT |
| | | | | | ISENLYRFFDNILLENKISEKYPDLAKELLPFKDAFTIQYFATRINQNAIDEYNHNIIGSSSENIDHN |
| | | | | | GVNSILNAYRQKHHLRTKDIPVMSRLYKQILSDSEEKFFFLQVTSKEQALALINDTTLSLKTSCK |
| | | | | | KLQDLFSTYVIEDNSSHIYLKTSQLHTISMSLYNRWDLFDVAIKDKASTLSTKDSNQLLAKYKDVI |
| | | | | | SIQELNNIFFNYYQSLDKDKQKEIGPIRNLTEYFQRAPKINISIDELKEKSITEFKTQLDTLLGPTHF |
| | | | | | YKAFHLYNGRKAISVPDRDISFYNEFEAAYRQLACASTTYDAIRNFATKKQYSLDKIPVFFGKSSL |
| | | | | | LISWENGYNAKSCLLFQQGNNYYLGILNKKLDETDIKKLHTDAIADPATRFIINSQKVDNKNVPR |
| | | | | | LFIHSKGDNLAPSVKKYNLPIQNILNLYTQGYYKTDYAKINPKKFKASLIKLIDYFKLGFSQHEDF |
| | | | | | KNFTFQWKESEEYNNINEFYHDVAVSCYAISKEHVNFSALKSLVKENKLYLFQIYNKDFSTHSHG |
| | | | | | TPNLHTLYWKALFDDRNNLNKVFKLNGGATIYLRKGSIAKKITHPKNQPIESKNPLHKKQSVFG |
| | | | | | YDLIKDKRFTEDKLFLHCPITINFKYPANIYLNNDVNNYLENHPEVNIIGIDRGERHLLYYTIIDQK |
| | | | | | GNILEQDTFNQIKYAYLDKKSNETVPVVVDYHTLLDNKQIQRTDARKAWETIENIKELKAGFLS |
| | | | | | QVIHQLAELVIKYNAIVVLENLNTRFKQTRVKVEKQVYQKFEKALIEKLNYLVFKDHQYDDLGS |
| | | | | | YAKGYQLTNPSDINQSGISQNGILFYIVPSYTSHICPKTGFVNLLTGKLHYKNIEASQEILKNFDGI |
| | | | | | KFNLANDYFEFKLDYRKFNIEMSQPCWTICTYGDERYAYTRTENNKTQVAKINVTQELKELFTK |
| | | | | | YEIDYTKGNNLLAKILELNDKSFFSSLLFLLNLTMQIRYTKPGTQDDCDYILSPIQYAKKSFFDSRF |
| | | | | | AQNNEPKNADANGAYNIALKGLKLICSIKDGALPKQEKGTERKEWFEFVQKKLYLDKD |
| MG29 | 6346 | MG29-23 effector | Protein | Unknown | MKDFTNCYQLSKTLRFELKPIGKTFDFIQEKGLLKQDEQRAESYKKVKKLIDEYHKAFIEKCLE |
| effector | | | | | TVFIPNKDIIEFESLFFKQEKDDKDKKELENLQKNLRTIIADSFRKSDNFKRLFGKELIKEDLLEFF |
| | | | | | KNEEELTLVGEFKDFTTYFIGFNENRKNMYDSDEKSTAIAYRLIHENLPKFLANKRTFDKIKTNY |
| | | | | | PKIIEDAKTIIEPELFGIPLEDMFSYKYVNQTFKQSDISLYNLMLGGKSDGNEKKQGLNELINLYR |
| | | | | | QTNELSKKDIPNLNVLYKQILSDRETFSFVSEKFENQNELLQSIQSFYTEQLLEWNNNDTTENVFL |
| | | | | | KLIQIIKEHENYDKSKMFLKNDIFITHISKQLFNDWSVIPTALKEQFYNTNPKLKQTETNDKKFEK |
| | | | | | IKFYSFFEIESALKDYCQDKDDFKGLYKDDILFSYFNNFKLKDKDNTLIENINQKYEEVETLLKTD |
| | | | | | YPENKSLISDDESIKKIKIFLDTLMDFLHFIKPLTAKGFIGEKEDAFYADFNVYFEQFENVTKIYDK |
| | | | | | VRNYLTQKPYSIEKYKLNFENSTLLDGWDQNKETANTSILFKKNGLYYLGVIDKKHNKVFENLIP |
| | | | | | ENTDNYFEKIVYKLLPGASKMLPKVFFSSKNINYYCPDENILKIRNHGTHTKNGEPQKGYNKLDF |
| | | | | | NIIDCRNMVDFYKKSIEIHKDWKNFGFQFSPTDNYNSIDEFYREVENQGYTIAYQKISKKYIDELV |
| | | | | | NQGKLYLFQIYNKDFSPYSKGKPNLHTLYWKELFSDENLKDVVYKLNGQAEIFFRQKSLQYTDE |
| | | | | | TLKKGHHYDKLKDKFDYPIISKKRFAFDKFQFHVPITLNFKAKGRDNINQNVLEYLKKTPKNDIH |
| | | | | | IIGIDRGERHLLYLSLIDINGNIKKQYTLNDIVNQYQGKTFATNYHNLLSEKEKSRADGRKEWKTI |
| | | | | | ETIKELKEGYISQVVHQVAKMIVEHNAILIMEDLNFGFKKGRFKVEKQVYQKFEKMLIEKLNFY |
| | | | | | VDKNKKKTELGGTLKALQLTSKFTSFREMGKQSGFIFYVPAWNTSKIDPVTGFVNYFYSKYENI |
| | | | | | KKAQEFFIRFSNISWNNDKNFFEFVVNNYTAFNPKAEGTRQDWVICTQGIRLENFRNQEKNNEW |
| | | | | | DTKEIDLNNDFKALFNKFKIDFSHDLQAQIVNQTEKTFFENLYHLFRLTLQMRNSRTGTDEDYLI |
| | | | | | SPIANDKGIFYDSRNYEKQENPVLPKDADANGAYNIARKGIILLDKIKKADLSKKVDLSQNNRDW |
| | | | | | LNFAQKIK |
| MG29 | 6347 | MG29-24 effector | Protein | Unknown | FKQTACIKQFLDSSRNLWAFVKDWNMEIKEIPEDAFTDWYDCIQNFVDNFPVINLYNKTRNHLT |
| effector | | | | | QKAYSKDKVKINFEKSTLLNGWDRNKESANFSIILERDGLFYLAVMTPGNNDIFGYDEIPGEIGK |
| | | | | | KKEKKENLRKLALAGKGENCYRKMNYKQIANVGKDIFTLCWDNKENIAIRKTKGREKIWGNQI |
| | | | | | TRIKETKSYSDNTEDRLVYFSYLIRCAKSYWKHFNLQLKRPEEYKTMQELLTCIGNQGYKISFDN |
| | | | | | IKESYIEENVDKGNLYLFQIYNKDFSRNKKAGGKDNLHTSYWKLLFDEDNLKDNVLKLNGQAEI |
| | | | | | FFRQASVKWSEEKMKKGHHYEKLKDKFDYPIIKDRRFTQDKFFFHCPITLNNKAPSNPARFNST |
| | | | | | VRNFLKKNPEVNVIGIDRGEKHLLYYSVVDRQGNIIEQNSFNTISTGFKPAGQSQEQKIDYRRLLD |
| | | | | | EKEKNRDKARKSWSAIENIKELKAGYLSQVVHKLAQLIINYNAIVVLEDLNYGFKRGRFKVEKQ |
| | | | | | VYQKFERALIDKLNYLVFKDRENRLQPGHYLNAYQLTNKFESFKKLGSQSGILFYTAASYTSTTD |
| | | | | | PVSGFMKNVYYTYSSIDKAVEFWKSFDSIIYNAEQNRIEFTYTLEKIMSKKLEKEKDEKSVEKTS |
| | | | | | WTVVSSVQRSKYIKKDRKTEILDVNTELKKLLDSNRIEYKDGADLRNRLAERNERGDASFHKSM |
| | | | | | VYYFNSILNIRASNPQAETGTGENDFIMSPVEPFFDSRKKYPGLPLDGDANGAYNIARKGIFMLNT |
| | | | | | LNNSENPEKENLNVSKKDWQNFAQADETVKRQKAKMK |
| MG29 | 6348 | MG29-25 effector | Protein | Unknown | MLKEFVGKYSLSKTLRFELKPVGKTIEHIEQKGLISTDEARAEDYKKAKELIDEYHKEFIHQALS |
| effector | | | | | SVRLIGLDSYEVLFLKQNRDEKDQKEFEKIQDDLRKQIVAGFKNHPHFKNIDKKELIKDDLPRFL |
| | | | | | QNQEDRDLIERFSSFTTYFTGFHENRKNIYSSEAKHSSIGYRVIHENLPIFLINKKAFLAINSNYPQI |
| | | | | | AQEAQSSLLEHLNGGIVEDMFANDYFSFTLIQTYIDIYNTMLGGKTLADGTKIQGLNELINLYRQ |
| | | | | | KHNIDKRELPNLKPLYKQILSDRDGMSWIPEAFECREDLNLAIQTFYNKNIVAFECCDGVVDITE |
| | | | | | KFLEVLTQTSSYDREKIFIKNDLSLTAISHVLCEDYRVIKDALWQKHLQENPKAIKSKDIAGDEER |
| | | | | | FFGRKNSYFSISEIAKALDLIEKPSDLFGYFKTEVEKQSKQVKSSFKEWELDPNNKKLTKEFLDST |
| | | | | | LDLQRTLKPLYVRSDIDKDIAFYALFDGYFDSLSAIVKLYDKVRNFESKKPYSTEKFKLNFENKG |
| | | | | | AFLGGWVDSYTDKSDNGTQSGGYLFRKKNAIGEYDYYLGISKDTKLFRSHLQNDIDENDISEYER |
| | | | | | LDYYQLKTASVFGNSYVDGSYSEDKIEIKNSIYNFVKSTDLGKELEDYISSKEAKEATPNKMINYI |
| | | | | | KEKNPTLYEELLEDEEFSKINKTVTKKLKETILSLHRVPKSQEYKEASFNLFTEPIEAIEKLSEEKT |
| | | | | | FVYFSISSREFENALANKDKPLLLFKITNKDLSYAETFLNGKRKSRGLDNLHTLYFKALMSGEQA |
| | | | | | VFDIGTGEVFYRKKSIEYSEEKMQKGHHYDKLKEKFSYPII |
| MG29 | 6349 | MG29-26 effector | Protein | Unknown | MKLNKFTHQYPISKTLRFELKPVGETADYIEDFKSQYLKDIVIQDQQRAEDYESIKVIIDEYHRHY |
| effector | | | | | IEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSIIEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNDLLKSL |
| | | | | | GKFIKEMVKEDGLFKKLEDSIKLITDADLHRTFIKNGVEITKISQSIFGNYSILKSAIYHHAESVLY |
| | | | | | PDPISGKISEALKEKRIKYVNKQVVFSIAELETILSNYASQMTDDNPDKEIIAQCENSEHPIRTYFL |
| | | | | | NAIENVKNDKDIEFGKAIENVLPLISLENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEEGKLFLFQIY |
| | | | | | NKDFSPYSKGKPNLHTLYWKALFDTENLKDVVAKLNGQAEIFYRKHSIKKDERTIHRANKSLQN |
| | | | | | KNENNPKKTSLFEYDIIKDRRYTVDKFQFHVPITLNFKMEKMTQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDSRNAPKDQPMDADGNGAYHIALKGLWNLEQIRNWDGESRLNLAMKNVDWFSFAY |
| | | | | | QKPFKK |
| MG29 | 6350 | MG29-27 effector | Protein | Unknown | MINSSKSIWDGFVNKYSLSKTLRFELQPISRTLDFIKEKGLIEQDKEREKEFNLVKKIIDSYYIEFIE |
| effector | | | | | NVLSKITIDSDLLKEYSAVYKNLKNDKYSSDLKKEFKLIQDKVRSEVYKQIYNFPNFKLLFGKELI |
| | | | | | KVILPKWLEYKNRLEDKELILKFDKWTTYFVGFFENRKNVFSKDPIPTSVIYRIVHDNLPKFLDNI |
| | | | | | EKFNKIKLLDNFDYFSIEKELSVELNNKNLDYYFNLSNFNLFLNQRGIELENTIIGGKSTENIKIKG |
| | | | | | LNELINLYSQKEKDLIKSKNIRKLKMSPLFKQILSEKQSFSDKFDLIKDNSSLITQINTFYTDEFNT |
| | | | | | NLPKILELISKLDQYDLDQIYINKNSITNISSNIFKDWSIISSGLKEYFIKNYNLSNKKIESRLNQKYF |
| | | | | | SISEIQEGVKLLNLDRINYNDFSDHFISDYFKNLINEKIIDEITNHKLDFDKINYNNLNSFSDNEKQL |
| | | | | | IKILLDSILGFYNSIKPLYVNIKSSQEEKTQEAYELDSDFYNDFQIIIDSFKKIIPIYNKTRNYLTKKP |
| | | | | | YTTKKFKLNFDNSTLLDGWDINKEKDNYSLLFKKDNQYYLGICSKGNSTDISKYIQKKVFNSGDY |
| | | | | | FEKIDYKLLPGPYKMLPKVFFSKTNIEYFSPSEEIISIRNYASYSKNGTPQKDFDKEEFNITDCHKLI |
| | | | | | NFYKFSLNKHHEWKNFNFNFKPTDQYKDINEFYQDVEDQGYNLSFKNIDSKYILDLVDSGKLFLF |
| | | | | | KIYNKDFSKFSKRTPNLHTIYWNELFSEENLSKLIYKLNGKAEIFFREKSNIKNNTIHGKNQLIQN |
| | | | | | KNPINNKTESIFEYDIIKDKRYTQDKFLFHCPITINFKSRGNGKDIHKQINNYIKDFEGNINILSIDR |
| | | | | | GERHLLYYTLLNSDGKIISQNSFNNISDGFNRSFDYQDKLDQREKERDQSRKSWTAIENIKYLKE |
| | | | | | GYLSRVIHEIAKIAIENNAHIVLEDLNFGFKRGRFKIEKQIYQKFEKMLIDKFNFLIFKKRSKESIGG |
| | | | | | ALNGYQLTNKFESFSKLGKQSGILFYVPASYTSKIDPTTGFFDLIRPKYESVDKSIQLIKKFEYIKY |
| | | | | | NSDMDMFEFNYNYFNFNNELKLDRKNWCIYSNGSRLYNFRNKDKNNEWDTKEINLTKELKDLF |
| | | | | | ESYSIDYNSTQNLIDRIILIDHKDFFEKLIYILKLMLQLRNSIPNSKEDYILSCVKNKDGLFYDTRKN |
| | | | | | MTSKSLPVNADSNGAYNIGIKGIMIIDKIKNNLEIKITKEEYVNFIINKNDYGK |
| MG29 | 6351 | MG29-28 effector | Protein | Unknown | MKNLSEFTNLYSLSKTLRFELRPVGETAERIEDFKNKALCDVVRRDEKRALEYVKMKKILDDYY |
| effector | | | | | RDFISYVLDQKIFTERDIKDAFEVYKKTRQPTQDRDKQKKEFQTVQKKLRDKTAKAFNERLKEK |
| | | | | | GLDEYSSLINTKGKDDKLKKPLLWHWLKKKYDGKLLSKEEFEDAEKTLKSFDKFTTYFKGLKQ |
| | | | | | NRDNMFSKKDQRTAISYRLINENMIKHFDNCMRLENIKKNHKSLYNEIKESANSLKPDSFMIFLN |
| | | | | | QTGIDNYNRIIGGDSIDQNKTGVNQKINLYRQKHNIKGKDLPLMAKLYKQILSKTEDKFVIDKFE |
| | | | | | SHKDMLDTIDEYMCNILDSKNIRAISSFIENHITPENMEYIFIKNDTTLTDISQFMFKDWGFIKRAM |
| | | | | | TKYSENEISGKKEREKWLKSDIFSLKDIQTSIDKYLVDLEEKDFTQTDIGLFFKSFINADGNIFDKI |
| | | | | | NESRKEAEPVIRSGEFNVNNERPDNKTDTDKIKNLLDSIMKLIHFLKPFHLVKKGKPIETDNADSD |
| | | | | | FYEPFNNSYNDLCLLIPVYNKTRNLLTQKPYSTDKIKINFDKGTLLDGWDVNKETDNLSAILLRE |
| | | | | | GKYYLAVMDKSSNMILTKENTSGFDLKNEDCYLKMNYKLLPNPSKMLPKVFFAEKNIEYFAPG |
| | | | | | DDIIRIRDKGLYKKEADDIESVHIWIDFCKESIKRHEEWNNYFNFNFRPTKKYSDVSGFYNEVAEQ |
| | | | | | GYALTYTPVSAKYIDDKVSKGELYLFEIYNKDLSIKKKNINGTPNLHTLYWKAIFNEDNLKDVVV |
| | | | | | KLNGEAEIFFRHASIDANSRVVHKAGTALYSKNPLNKKNSTFEYDIIKDRRFSKDKFFFHCPITLN |
| | | | | | FKAQGEKRFNERVNRFLENNDDIKFIGIDRGERHLLYYSVIDGRGRIIEQDTFNVLKNSYESNGSI |
| | | | | | VEKKTDYRDLLDRKEKERDEARKKWSAIENIKELKSGYLSHIIHELAKLMIKHNAVIVLEDLNFG |
| | | | | | FKKGRFKIEKQVYQKFEKALIEKLNYLVFKNEKPGNAGYVLKAYQLTDEFESFDKLGKQSGFLF |
| | | | | | YVPAGYTSKIDPATGFVNLENTYYENIDKSKEFFGKFDSIRYNKDRDYFEFAFDYKKFTDRSGGK |
| | | | | | TKWTVCSFGNERYYYDARSRSYVCHDITRNLKLLFGHLKYENGENIIEKILEQTEAGFFKSLYFN |
| | | | | | LKVLFSLRYTGKDDKGNEFDYILSPVGNFFDSRKADENMPLDADANGAYHIALKGMMTVKGIR |
| | | | | | DGKLPKTEKGMMNKEWFAFVQERNMKN |
| MG29 | 6352 | MG29-29 effector | Protein | Unknown | MSAQSALSTLINKYSLSKTLRFELIPIGKTKESIDRKGLLSQDVKRAQSYKEVKKIIDEYHKEFIEK |
| effector | | | | | SLINAKLKGLEEFSKLYYKLQKEDKDKKNIKKMQDNLREQISDLFKNNKKDKWNILFKEDLIKK |
| | | | | | ELPLFAKDDKQKNLINEFNKFTTYFTGFHKNRKNMYAEEEKSTSIPYRIIHQNLPKFLDNIRIFEKI |
| | | | | | KKNKINTDVIEKELSLFLNGIKINDIFSINFFNDVLNQKGITFYNTILGGVSEKDRTKIKGINEYVNT |
| | | | | | EYNQKQLDKKSKIPKLKQLYKQILSDTETASFVLEQFENDNQLLEKIEQFYNTELINYETEGKTQ |
| | | | | | SVFLQFEQLFKNMQNYDASKIYISNLSIANISKIIFGDWSIICNALAEWYDKHNTKGKKINEYKKE |
| | | | | | NFLKQDFSIQQIEDAVLEYKNDTLNKEINFLLNYFASFLNEKSKKNIIQRIETEYSKVKDLLNTDYP |
| | | | | | EKKKLASDKDNVSKIKAFLDSLMDFLHFVKPFNIKKDTGLEKEENFYSIYVPLFEQIDKIIPLYNK |
| | | | | | VRNYLTKKPYSTEKIKLNFENSTLLDGWDLNKESDNTSVVLRKDDLYYLGIMDKKHNRIFKELP |
| | | | | | SQNGNESSYEKMIYKLLPGPNKMLPKVFFSKKGKKQFKPSKKLLKKYEDGTHLKGDNFNINDC |
| | | | | | HNLIDFFKESIAEHEDWKQFDFKFSSTSSYKDLSNFYKEVEKQGYKITFQNISENYINQLIDEGKL |
| | | | | | YLFQIYNKDFSKYNKGTPNLHTLYWKMLFDNDNLKNIVYKLNGKAEVFYRKSSLILGDNIVHKA |
| | | | | | GEAIINKNPDNEKKHSTFDYDLIKDKRFTLDKFQFHVPITLNFKSEGRQNLNEDVRKFLKNNPDI |
| | | | | | NIIGIDRGERHLLYLTLINQKGKILFQKSLNEITNEYNNKNGKSQIKSTNYHSLLDKKEKKRDEAR |
| | | | | | KNWGIIENIKELKEGYMSQIVHYISKLMIEKNAILSLEDLNFGFKRGRQKVEKQVYQKFEKMMI |
| | | | | | DKLNYLVFKDKKANETGGLLNALQLTNKFESFAKLYNQSGFIFYVPAWNTSKIDPITGFVNLLKP |
| | | | | | YYENLNKSQEFFKKFNNIKYNPKQEYFEFNFDYKNFTNKAEGSKNVWEICTTNNERFMWDKTL |
| | | | | | NSGKGAQKAVDVTQELKKLFDSSKINYLNGNDIKEDIINQNSADFFRKLMKLLSVVLSLRHNNGL |
| | | | | | KGKDEKDFILSPVEPFFNSLNAKMEEPKDADANGAYNIALKGLLILKQINESEDLRKIKENLSNKE |
| | | | | | WLKFAQSKSF |
| MG29 | 6353 | MG29-30 effector | Protein | Unknown | MQECRNNCRRLMALFDFEDIKGEKLMALIADEFLGQYSLSKTLKFELVPQGKTKDLIKNINDSIL |
| effector | | | | | AIDAKRAAEYKNVKKILDDYYRFFIEQVLEKNIFEKSEVEEAYIAFQQRAKDNKAFEKTQDNMR |
| | | | | | KKIAKALKDGRSGSQLDAYEKLFKTDDKSELYKWLNYGKDRKELTEELYESYKKSLQQFDKFT |
| | | | | | TYFTGYKDNRENLFSAEEKSSAISYRIVNENMVRFFENCQRFDDIKKKHAGLYEQLEVNQAIFQY |
| | | | | | NKFSELLGQSKIDEYNQMIGFSIENSDTKGINSLINEYRQKNHIRNKELPMMVQLYKQLLSDREK |
| | | | | | SFVIDEITSDEEMEEKAIECCREAREIEKKLALLVKEYVNEDNTVRIYLRGSKLTDLSQNIYGQWD |
| | | | | | IINKALLMRLESLTTKKQREEFDKRTKKVININELQGILQEYLAGLDSEEYKKIQDKVTISELIVE |
| | | | | | NIPAVEYSPVLNGLRFGSKEEKINKIKGVVDQIISMLHYYKIFYLYEGNKQLEVAEKDAFFYSEFD |
| | | | | | ALYNALSLATKVYDHVRNYVTKKPYSENKIKVNFNAPTLLNGWDINKEESNLSVLLEKNGLYYL |
| | | | | | AIMDTNHRKCFDLKDIAVAKAAFSDVNGAYFNKIEYKQVTGANKMLPKVFFAESNIDYYAPSSEI |
| | | | | | RTIREKGLYKKDANNIEARWQWIEFCKQSITKHPEWNNYFKFNFKPTKNYMDVNGFYRDFDNQ |
| | | | | | AYSIKKVRISEKYISDLVAAGQLYLFQIYNKDFSQYSKGKQNLHTMYWRMLFDSQNLKNIELNA |
| | | | | | NAKIFKLNGEAEIFFRRQSLEKKITHAKDMSIANKNPHNPKKQSTFEYDLIKDKRYTENKLFFHC |
| | | | | | PITINFRASSLPAQFNKKVNKFVANNPDINIIGIDRGERHLLYFTIINQNGEILKQGSLNHIKDNYIS |
| | | | | | NGKEVPIDTDYHELLDRKEKERDAARKNWTAIENIKELKAGYLSQVVHQLAELMIKYNAIVVLE |
| | | | | | NLNAGFKNGRVKVEKQVYQNFEKALINKLNYLVFKDCSLNKPGGVLNGYQLTAPFDSFRSLGSQ |
| | | | | | SGFLYYVYPSYTSHICPKTGFVDLLHPKHQSVAEAQKFFEKFEFVRFNQDKQYFEFGLDYNRFG |
| | | | | | KQMNKNRWVVCTYGEERYGFDGKEMTAKKYNITEEIRALLDKAKIVYDNGCDIKNAICTQDDK |
| | | | | | SFFKSLLYYLCLTMQMRNTNGGINDDNDYILSPVRDKNGNFFDSREATDTEPKNADANGAYHIA |
| | | | | | LKGLKLISSIDEEGKLVLKKTETQDWFNFAQEKPYLK |
| MG29 | 6354 | MG29-31 effector | Protein | Unknown | DSISDKDDAFYSQFAPLYEQLNKLIPLYNMVRNYLTQKLYCTDKIKLNFENSTLLDGWDVNKEP |
| effector | | | | | DNTSVILRKDGLYYLVIMDKAGKKVFMDVPKIAYSGTFYEKMNYKLLPLVNQQLPRVFFAKSRI |
| | | | | | EFFKPSEAIQENYKKETHKKGDTFNIKDCHALIDFFKASLAKHEDWKHFNFKFSPTKSYQDLSGF |
| | | | | | YREVEHQGYKMSFENIPTDYIDKMIEEGKIYLFQIYNKDFSAFSKGLPNMHTLYWKALFDENNL |
| | | | | | ADVVYKLNGQAEVFFRKSSIEEKNKVIHKAHELLKSKNPNTPNNNNTFDYDLIKDRRYTVDKFQ |
| | | | | | FHVPININFKASGSEIINAQTNDFLKNNRDVKIIGLDRGERNLIYLTLIDQKGNIIIQESLNTISNKER |
| | | | | | KIETPYHTLLNIKEKERDAARKSWNTIENIKELKEGYISQVVHKIAEMMVKHHAIVVMEDLNFG |
| | | | | | FKRGRFKVEKQVYQKLEKMLIDKLNYLVFKDAQPTQPGGLLNALQLTNKFESFKKMGKQSGF |
| | | | | | LYYVPAWNTSKIDPATGFVDFLKPKYENVEKAKAFFSKFDSIKYNTVKDYFEFAFDYKNFTTKAE |
| | | | | | GSKTDWIVCTHGDLRFRYNAQTKESEAVNVSQEIKKALKKHEITFEQGKDFKNLLIAKEGKEIFS |
| | | | | | ALLHLLALTLSLRQTKSGSEIDFILSPVTNRKGVFFDTRNADEKMPIDADANGAYHIALKGLWCL |
| | | | | | KQISQSDDMKKVKLAISNKEWLEFVQNKRF |
| MG29 | 6355 | MG29-32 effector | Protein | Unknown | MFTNLYPTSKTLRFSLIPHGDTLNNIEKAGILTEDEKLAEDFKKVKKIADDWLKNFINESLAGVSL |
| effector | | | | | SLENLLIYEEKYNLFPRNEKDEEEFNDIKTKLRKEVVSYLAKNPKFKLLGSADLIRKELPEFAKTE |
| | | | | | EEKNLINKFKTFTTYFVNYYKTRENIYSAEEKHASHAYRVINENLPLFITNKKNFDIIKNSYPELIE |
| | | | | | DIKKSVEPLLNGEKVEDMFSTEWFSKTLTQSGIDLYNKMIGGESLEDGKKIQGFNEKVNLFRQA |
| | | | | | NKLDGKSVPMLKQLKKQILGDKNVPAWITEGFKNKDSMNNAIVEFMDNIKPVLSTAADAFVTEE |
| | | | | | SHDYNKIFIKSRFLTDLSHELFKDWNFLKNILLEKYTAKNPKSKNPEKEFAKISYFSIAEIQAILPN |
| | | | | | LSKDFIFKFLYNKTINIVAEIRQSYELWNTNQKNVPVLKSLMDNIIQLHRTFKPFDIDEADKDPVF |
| | | | | | YELFDRIFEGIDGAVKLYNEVRNFITKKPYSLEKIKLNFGNSTLLAGWDVNKESDNSSILLRKGND |
| | | | | | YYLAIMNKSHNKVFKNAPLVKNNEESYKKMEYKLLPKSYMMLPKVFFSEGNKHKYEPSDEIMR |
| | | | | | IYENGTFKAGDNFNADDLHALIDFYKDSIEKNPEWACYKFNFRPTEEYQKINEFYDDVDSQGYVI |
| | | | | | TFRDIKASYIDELVKDGKLYLFKIYNKDFSVYSKGTSNLHTLYFKMLFDERNLKDTVYKLNGGA |
| | | | | | EMFYRKKSLNYSEEIMKNGHHVEELKGKFDYAIIKDRRFAFDKFQFNVPITLNPN |
| MG29 | 6356 | MG29-33 effector | Protein | Unknown | MSKIYQQFTRLYKIQKTLRFGLKPVGETANAIDDFKSQYLQDVVQEDGQRAEDYKVVKDLIDDY |
| effector | | | | | HRVYIEEKLSQPVDRATGEMWVTPEHLEAAYYDYQNLKNNDPKDNKIKKAWAETQKSLRKQL |
| | | | | | VKSFSDNSDLFRKKLITRDLPAFLKGQGKWDENEKAVKSFNKFTTYFKGFHENRKNMYSDEDQS |
| | | | | | TAIAYRVMNENLPKFFNNYLSYQKIKDKLKFSVEKELFTKMGISGIGDIFQPRYFIKLFTQSGLDN |
| | | | | | HQELLGGKTNEDGRKIQGLNEQINLYNQQQSDRQNKLPRFTSLYKQILSDREAHSFIPEVFKDDQ |
| | | | | | ELLKTLQGYIEKATKKEGLLDNLEKSIALLSTADNEKVYVKTVGLTDISSALFGSYDIIGAALSHH |
| | | | | | AENTAHQNHTKKPASKTLIKKRESFCKQDVFSTAKLDEMITAYIAQLEKTDPLHQQLKKLKTPK |
| | | | | | RPIQIYFLEAFQQAKKEYGFDACIKNITPLLSLESLSKKRQAPTSEGEQGDKGYQQLHSIQKMLD |
| | | | | | AFMAISYKLKPLHLVKGRKAIDMPDMDMRFYTKFSESYEDYSDTIINLYNKARNHLTKKPFSKD |
| | | | | | KIKLNFGNPTLLDGWDANKETDNSSLIFEKDGFYYLGIMHPKHKDLLNYITGIDDIGNDKKTKK |
| | | | | | KELLKKNIEANKNEQHYRKIVYKLLPGANKMLPKVFFSGKRQDYFSPSSEILRIRNSASHSKNGN |
| | | | | | PQEGHAKAEFNIDDCRKIIDFFKVSISKHPEWRAFDFQFSPTQNYQDLSDFYREVEQQAYRVDFD |
| | | | | | LIKQSYIDECIIKGKLFLFQIYNKDFSPYSKGKPNLHTLYWKGLFDPENLKDVVLKLNGEAEVFY |
| | | | | | RPSSINVADRTIHRANEAIDNKNKEFHRKSTSTFAYDIIKDRRYTQDKFQFHVPITLNFKDQGKPH |
| | | | | | FNDKVNLKLRNTKDTHVIGIDRGERHLLYYTVVNSKGEIIEQDTLNNISTDQGYAIDYQNKLHKR |
| | | | | | EKERDAARKSWSNIENIKELKAGYLSHVVHKLAELIIKHNAIVCLEDLNFGFKRGRFSVEKQVYQ |
| | | | | | KFEKALIDKLNYLVFKNSTLQKPGHYLNAYQLTAPFESFEKLGKQSGILFYVQAAYTSKIDPSTG |
| | | | | | FIDFLKPKYKSLSASKEFFETMSSVTFNKAKDYFEFSFDYKKFNPSQKFGSYTTAWKACSFGKIR |
| | | | | | YHNKRNNKGKWETCSINVTEELKKLFDNADIQYQTGQELKESLSLVKDTKFYKTLFWLLRLLLS |
| | | | | | LRHSKTGTDDDFILSPIADKNGDFFDSREAKDGKPKDADANGAYNIALKGLWNLQQIKQWDGKS |
| | | | | | SLNLAMKNEDWFSFIHDWHNQ |
| MG29 | 6357 | MG29-34 effector | Protein | Unknown | SSFIPDQFENDNDLLKSLGKFIKEMVKEDGLFKKLEDSIKLITDADLERTFIKNGVEITKISQSIFG |
| effector | | | | | NYSILKSAIYHHAESVLYPDPISGKISEALKEKRIKYVNKQVVFSIAELETILSNYASQMTDDNPDK |
| | | | | | EIIAQCENSEHPIRTYFLNAIENVKNDKDIEFGKAIENVLPLISLENLNKGKDGQAQTHKIQKMLD |
| | | | | | AFLAVTHAVKPLHLVKGRKPIDIPDMDMGFYAEFSKAFENFEQLVITLYNKTRNHLTKKTFSTD |
| | | | | | KIKINFENPTLLDGWDANKEKDNSGVLFEKDGNYYLGIMHPKHKNIFNYIKGINDIESEKRSLSK |
| | | | | | DELFNKIVDGESEHYQKIVYKLLPGVNKMLPKVFFSGRRIDFFAPSTEVLKIRNSASHSKNGSQQ |
| | | | | | KGFEKEDFNLKDCHTIIDFFKKSIEKHPEWKEFEFEFSPTSSYEDLSGFYREVEHQGYKMDFHPI |
| | | | | | KKSYIDQCIEEGKLFLFQIYNKDFSPYSKGKPNLHTLYWKALFDTENLKDVVAKLNGQAEIFYR |
| | | | | | KHSIKKDERTIHRANKSLQNKNENNPKKTSLFEYDIIKDRRYTVDKFQFHVPITLNFKMEKMTQF |
| | | | | | AHNEKVNQMIVKSENTHVIGIDRGERHLLYYSVINPKGHIVEQGTLNTISTDKGYEVDYQQKLDS |
| | | | | | KEKARDTARKSWTTVENIKELKAGYLSHVIHKIAFLIEKYNAIVCLEDLNFGFKRGRFKVEKQV |
| | | | | | YQKFEKALIDKLNYLVFKDAKPNEPGHVLNAFQLTAPFDSFKKLGKQTGLLYYVQASYTSKIDP |
| | | | | | VSGFINFLYPKYESLLKSKIFFESMDGIRYNADKDYFEFSFDYRKMTPNRNLEGYQTKWTACTFG |
| | | | | | EKRFKNIRNAHGNWESVEVNVTEALKKILKNEDVDFKSGHDLRFEISKVKSTKFYKKLFKLLQIT |
| | | | | | LSLRHSKTGTDEDFILSPIVDENGKFFDSRNATKDQPMDADGNGAYHIALKGLWNLEQIRNWDG |
| | | | | | ESRLNLAMKNVDWFSFAYQKPFKK |
| MG29 | 6358 | MG29-35 effector | Protein | Unknown | MGMIGDQFIGQYFLQKTLRFELRPIGETQKLLRDFKEGVQGNLLEYDAERARAYPTVKKILDDY |
| effector | | | | | YRYFIDQVLSGFAFDSQTINEVFEMYKKAKKDAEAAKEYAVHTKKLREQLSAAFKALITYYMLD |
| | | | | | KYEHLFNRNRESRLFEWLDIRFENDHLTENEYDEIKDVLDKFDKFTTYFTGYKENRANLFVADE |
| | | | | | KATAIAYRVVNENMPRFFENCIRMENIKKRHSDLYKLLVSFEGYFVPQAYANIICQPAITDYNKII |
| | | | | | GRPTQNPDEKGVNSIINEYRQKNQIKNRELPMMAQLYKQLLSDRVTVFSDPVINNDEEMQSIIAE |
| | | | | | TIEIARGLFSEVINLTAIHALADNSENIYINSSALANLSHRFCDDWNLIYRACEAKMIKLSGKQKK |
| | | | | | GLENKLKMAIPMSELQNIIEEYIATLDEELKLSYNNIPVLCDYFQNPPLDDFESATLKFEQIVKTT |
| | | | | | MSRTDLIQAIKEVLDKAMEVVRFFKPLYLFKGRSPLEVPDRNEDFYNEFERLYAELNLISKIYDR |
| | | | | | VRNYATKKQFSQDKIKLNFNNPTLLDGWDLNKEQDNLCVILIRDGNYYLALMNRDYRRLFDLK |
| | | | | | NDEVRNKALGKAGDHCYSKLEYKQVTGASKMLPKVLFAATNSDLFKPSQEILDIRKAGSHKKE |
| | | | | | AGNIEALHKWIDFCKQSIATHPEWNDHFDFKERSTSEYSELTEFYNDFDRQAYKIKFVDIKVEYID |
| | | | | | QLVKEGKLYLFQIYNKDFSPYSKGRPNLHTTYWRMLFGNENLANITMDPDRPIFKLNGEAEIFFR |
| | | | | | KASLEKQITHAKGQPITNKSKKDNGKKSESIFEYDLIKDKRYTEDKLFFHCPITINFRAPGTTVGS |
| | | | | | FNRKVNCFVERNPEVNIIGIDRGERHLLYYTIIDQKGNILEQGSLNQLHNSYTSAGRVVEHNINYR |
| | | | | | DLLHEKEKGREEARKNWETIENIKDLKAGYLSQIVNLMSNLMIKYNAVLVLEDLNAGFKRSRIK |
| | | | | | VEKQVYQKFEKAMIDKLNYLVFKELPPGSSGHYLNGYQLTAPFTSFRDLGRQSGFLYYVYPSYT |
| | | | | | SHICPKTGFVNLLNTRYESIEKAISFFEKFNRIKYNPGSDYFEFDFDYASFGKDVARSQWCVCTAG |
| | | | | | EKRYYYANHDKTSRECNATQQIKELLDKYNIEYIRGKDLLPEIIKMNDKGFLNGLMFLLGVVLQ |
| | | | | | MRYTVSGTSNDDDFILSPVMDEQGQFFDSRSAATSEPQNADANGAYHIALKGLKMISSISDGKLK |
| | | | | | TVNKNERQDWFAYVQNKMYR |
| MG29 | 6359 | MG29-36 effector | Protein | Unknown | MNSIFEQFTQQYPLSKTLRFELKPVGNTAKLISEFNESLPESSVAKDEEKSNAYPLVKKILDDYYR |
| effector | | | | | DFINEVLSKSDLDALKIKQAFNCYKECQSKNAAADAESKKAEYKAQKSILRKDTASFFSSESLKLS |
| | | | | | AMFKGTKKSCAIYEYGELFKEASPLFIWLQNRLEKQVISQEEFDRQAGLISKFNGFTTYFTKYKA |
| | | | | | NRENLFANEEKASSIAFRVVDENMEKFFDNCIAYKKITQKYPSEELAAELIKCEQFFTPENYGICL |
| | | | | | TQSGIDVYNQIIGKKSDDTYGKGINQQINEFRQKNALRRNDAPLMTVLFKQLMSESERVEVIETI |
| | | | | | DSDEELFSVVKDAYNTCIALVEGLSILCDSSLTDENLSDIFIRPDGLSNLSQKVFGKWDIIDSALNM |
| | | | | | KKESIGQKKFDAKYSKVISLLDLQTMCDAYISVIDDSEIRRNCTFSDYFKTFESSLIKSAYLEAEEV |
| | | | | | LNSIGLDKDKSMPTNDTDLGGKGFRQLQKIKQLLDSINEAVHFYKPFLLEKEGKAIEAEENNKEF |
| | | | | | YNEFLLNYRDLSVFPKIYDKVRNYATKKPYSKDKFLLNFDKPTLLDGWDVNKEESNLALLFIKD |
| | | | | | GKYYLGIMKNPRLFSNLPVKGIANENEPAYEKVIYKQVSGACKMFPKVFFADLNRELYKPGSEI |
| | | | | | QRIREEKSHLKGGSEDSKNKWIQFCIDCIDKQPEWKTYFKFNFKEPQEYPDVNSFYKDADAQMY |
| | | | | | SISFTKISCEYVNDAIKKGELFLFELYNKDFSEYSKGRPNLHTLYWKMLFDENNLHNIMNNPDKG |
| | | | | | IFKLNGEAEMFYRKASLPDKATHPANKPITNKNPLNQKMQSTFEYDIKKDRRYMSDKFMLHCPL |
| | | | | | TINFRKEKVGQGQFNNKVNTTIEQNLDDVKIIGIDRGERHLLYYTMINSKGEIIKQGSLNSVTADC |
| | | | | | GVTTDYRSKLDDKEKSRTAGRENWGQIETIKELKQGYLSQVVHMISQLMIENNAVIVLEDLNSG |
| | | | | | FKNGRKKVEKQVYQNFEKALIDKLN |
| MG29 | 6360 | MG29-37 effector | Protein | Unknown | MKNNFNEFVGMYSLSKTLRFELIPQGKTLENIQKKGIIATDTARNESYKEMKKTIDEYHKDFINQ |
| effector | | | | | ALSEAKLNKLDEYYQLYNLNAEKKKDENFKKKFDDVKKELRKEIANSFKSGAVKGIFERIDKKE |
| | | | | | LIKEDLENWINENNNNKYFDKDFKTFTTYFKGYHENRKNMYSDEEKSTAIAYRLINENLPKFIDN |
| | | | | | LNIFEKVKNSQVAENFEAIYKDLEAILNVNSIQDIFTLNYFNEVLTQPQIEAYNAVIGGKSENELKI |
| | | | | | KGLNEYINLFNQKQTEKSDKIPKLKPLFKQILSEKLHISFLPEAFESTDEMLTAIEKYYKNNLILC |
| | | | | | DINNNGKQLNILCEIQKILADLKEYDLAKIYLRNDTKITNISQKIFGNYNIIGEALSNYYDKIIKPDF |
| | | | | | ENLYQKADEKKREKLDKKREKLDKEKEKFAKKDYIAIEELQKSIDLYIADFDNSDENKSVKERY |
| | | | | | SKTCIADYFKNHFFAETKEEKGKSFDFISNISAKYNCVKGILNISFNNSDLTMEQKYNIKLFLDSIM |
| | | | | | EMLHFVKPIYLKSDEISQKDENFYSTFDPLFEQLSLVTKLYDKVRNFVTKKPYSIEKVKLNFDCST |
| | | | | | LMDGWDENKESSNSSILFMKGGSYYIGIMDKRNTHIFEDIEETFEENNYAKIVYKLLPGPNKMLP |
| | | | | | KVFFSKSRIDEFAPSNEILEIYEKESFKKGQEFNINDCRKLIGFFKDSIQKHSDWKKFNFVFSDTSQ |
| | | | | | YNDISEFYKEISQQGYKITIKYISNKYIEQMINDGKLYLFQIYNKDFSSYSKGKPNMHTLYWKALF |
| | | | | | DEENLKNVVYKLNGQAEIFYRKASIEEKNKTTHKANKPINAKNPKTPNKTNIFEYDIVKDKRYTI |
| | | | | | EKFQFHCPITINFKANENPKVNSKVFEYLKNNPDVNIIGIDRGERHLLYISVIDQKGNVLKDKNGK |
| | | | | | SIQYTLNEIVGQYKNSKGETVDFKTPYHTLLDIKENEKAKARENWSTIENIKELKEGYISQVVHLI |
| | | | | | SKLMLEYNAIVVMEDLNFGFKRGRFKVEKQVYQKFEKMLINKLNYLVFKDKAPNEIGGLYKAM |
| | | | | | QLTNQFKSFKEMGKQNGFIFYVPAWNTSKIDPTTGFVDFLKPHYKSIEDSREFINKFDSIRYNKEK |
| | | | | | DYFEFAFDYDNFTTKAEGTRTQWTVCTYDIERYAWNKSLNQNKGDYEKINVTQKIKELFTENNI |
| | | | | | EFSSGNDLKRLITNINNSKFYSKLLKYLSVTLSMRYSSSKDGKDFILSPVINSNNEFYYSENASKEL |
| | | | | | PQDADANGAYHIALKGLWVLNEINNTDDFKKLRIAISNKEWLNFAQDIAKRK |
| MG29 | 6361 | MG29-38 effector | Protein | Unknown | LGQSKIDEYNRMIGFSIENSDTKGINSLINEYRQKNHIKNRELPMMVQLYKQLLSDREKSFVIDEI |
| effector | | | | | TSDEEMDEKATECCLEVREIEKKIALLVKEYVTGDNTGRIYLRGSKLTDLSQNIFGQWDIINKAL |
| | | | | | QMKLETLATKKHKEEFDKRSKKAININELNDILQEYFIGLDSGEYKIMQEKPALSELIIENIPVVE |
| | | | | | YSPVLNGLGFGTKEERINKIKGVFDQIISMLHYYKIFYLYEGNKQLEVAEKDAFFYSEFDGLYND |
| | | | | | LSLATKVYDHVRNYVTKKPNSEIKIKVNFNAPTLLNGWDINKEESNLNVLLEKNGLHYLAIMDT |
| | | | | | NHRRCFDLKDIEVAKVAFCDLDKPYFNKIEYKQVTGANKMLPKVFFAESNIDYYAPSSEIRTIRE |
| | | | | | KGLYKKDANNIEAMWQWIDFCKQSTEKHPEWNKYFKVNFKPTKNYMDVNGFYRDFDDQAYSI |
| | | | | | KKVRISEKYINDLVAEGQLYLFQIYNKDFSQYSKGKQNLHTMYWRMLFDSQNLKNIELNANAKI |
| | | | | | FKLNGEAEIFFRRQSLEKNITHAKDMPIENKNPHNPKKQSTFEYDLIKDKRYTENKLFFHCPITIN |
| | | | | | FRAASLPVQFNKKVNKFVANNPDINIIGIDRGERHLLYFTIINQKGEILKQGSLNHIKDNYISNGKE |
| | | | | | VPVDTDYHELLDRKEKERDAARRNWTTIENIKELKSGYLSQVVHQLAELMIEYNAIVVLENLNA |
| | | | | | GFKNGRVKVEKQVYQNFEKALINKLNYLVFKDCSLNQPGGVLKGYQLTAPFDSFRSLGSQSGFL |
| | | | | | YYVYPSYTSHICPKTGFVDLLHPKYQSVAEAQRFFERFEFIRFNQDEGYFEFGLDYDRFGKKMN |
| | | | | | KSKWIVCTYGEERYGFDGKDMTAKKYNVTDEISALLDKVKIVYGGGRDIKNDISTQDDKAFFKS |
| | | | | | LLYFLCLTMQMRNTNGGTNDDNDYILSPVQDKKGNFFDSREANDTEPKNADANGAYHIALKGL |
| | | | | | KLISSIDEEGKLVLKKTETQDWFNFAQEKSYLK |
| MG29 | 6362 | MG29-39 effector | Protein | Unknown | YDEIYQDAILPLKDILLHLAHYDLNRVYLKNDTGITNISQKIFGDWGVITKAIVKSFTKQYKGKA |
| effector | | | | | KPGTEKWDSELNKYQKGFESFSINFINESLLLLESPEYHFSIEKYFIIGGKCKSGANMFQAVEDKY |
| | | | | | SIARQLLNNVYPEDKNLSQQQGDVDKIKQLLDSIKDLQWFLKPLLGSGKEPEKDERFYGEFSTL |
| | | | | | MDLLDQVTPLYDKVRNYMTAKPYSTEKIKLNFDNSLFLSGWARDYDTKAGLLFFKDGKYYLGI |
| | | | | | NNKKLTVDEKNELNSSNSALSGKRIILDFQKPDNKNIPRLFIRSKGDSFAPAVAKYNLPINEVIDIY |
| | | | | | DSGKFKTEYRKTNEADYKKSLSRLIDYFKEGFSKHESYNHYSFCWKETSQYKDISEFYKDVEVSC |
| | | | | | YQVLEETINWESLMEFVETGKIYLFQIYNKDFSSYSSGTPNLHTLYWKILFDKENTQDVVYKLNG |
| | | | | | QAEMFFRKASIQHKNRILHKANRPVDNKNELNPKRQNVFQYDLIKDKRYTVDKFQFHVPITMNF |
| | | | | | KATGINNINPLVNEFIRDNNDVHIIGINRAENHLLYIVVIDSKGHIVKQFSLNEIINEYNGNKYHTN |
| | | | | | YSQLLNKREAERNEARLNWSTIEGIKTLKEGYLSQVIHQICQLIVQYNGIVVLEDLNMEFKQGRQ |
| | | | | | KVEKSVYQQFEKKLIDKLNYLVDKKKSLSEVGGTLKALQLTNKFESFQKMGRQSGFLFYVPAW |
| | | | | | FTSNIDPATGFVNMIDTRYQNIEKSKELFSRFADIRYNAEKEYFEFEIKDYTQFNPKSEGTRQNWII |
| | | | | | CTFGTRIEKFRNPDKNNQWDSREIDVTECFKALFEQYNVNFNENLKEQIVNINDKAFFEQMLDFL |
| | | | | | YLTLQMRNSEIGNAGSDYIISPVCDPNGRFFDSRTAGHQYPENASANGAYNIARKGLYYVNQIKQ |
| | | | | | ADDIRALRLGLTNNEWLKFVQD |
| MG29 | 6363 | MG29-40 effector | Protein | Unknown | YANAKTENQIKHLNDTKNKWNSDFISLGLLQKTLAKYIETLDADSEIRKIYTPTIITDYFKRHIIKK |
| effector | | | | | EVEEAKNNETIKKTTDVELFYSITGQYLGVKGLLNIEKTDNKTLAQEKEKVHQLKSFLDSILELN |
| | | | | | HFVKPLFLTDDSISDKDDAFYSQLAPLYEQLNKLIPLYNMVRNYLTQKLYCTDKIKLNFENSTLL |
| | | | | | DGWDVNKEPDNTSVILRKDGLYYLVIMDKAGKKVFMDVPKIAYSGTFYEKMNYKLLPLVNQQL |
| | | | | | PRVFFAKSRIEFFKPSEAIQENYKKETHKKGDTFNIKDCHALIDFFKASLAKHEDWKHENFKFSP |
| | | | | | TKSYQDLSGFYREVEHQGYKMSFENIPTDYIDKMIEEGKIYLFQIYNKDFSAFSKGLPNMHTLYW |
| | | | | | KALFDENNLADVVYKLNGQAEVFFRKSSIEEKNKVIHKAHELLKSKNPNTPNNNNTFDYDLIKDR |
| | | | | | RYTVDKFQFHVPININFKASGSEIINAQTNDFLKNNRDVKIIGLDRGERNLIYLTLIDQKGNIIIQES |
| | | | | | LNTISNKERKIETPYHTLLNIKEKERDAARKSWNTIENIKELKEGYISQVVHKIAEMMVKHHAIV |
| | | | | | VMEDLNFGFKRGRFKVEKQVYQKLEKMLIDKLNYLVFKDAQPTQPGGLLNALQLTNKFESFKK |
| | | | | | MGKQSGFLYYVPAWNTSKIDPATGFVDFLKPKYENVEKAKAFFSKFDSIKYNTVKDYFEFAFDY |
| | | | | | KNFTTKAEGSKTDWIVCTHGDLRFRYNAQTKESEAVNVSQEIKKALKKHEITFEQGKDFKNLLI |
| | | | | | AKGGKEIFSALLHLLALTLSLRQTKSGSEIDFILSPVTNRKGVFFDTRNADEKMPIDADANGAYHI |
| | | | | | ALKGLWCLQQISQSDDMKKVKLAISNKEWLEFVQNLCWH |
| MG29 | 6364 | MG29-41 effector | Protein | Unknown | MKSTLDQFSHLYPMSKTLRFELIPQGATTANIESRGFLKKDEERAESYKKMKETIDRFHQDFIEK |
| effector | | | | | AMAHVRLSNLEDFENLYNAPNEEKKEDKYKKQLEKVQERLRKEIAKGFKSGEVKAIFLKIDKK |
| | | | | | DLVTKLLEKWIEENNLEDVHFDPEFKKFTTYFSGFHQNRKNMYTDKAQSTAIAYRLVHENLPKF |
| | | | | | IDNINIFKKVSEIPELKQNLEKLYKEIEEYLGIVSIEEAFELGYFNEVLSQKGIDVYNLILGGRSEKE |
| | | | | | NKKKIQGLNEHINLYNQKQDKKNKIPKLKVLYKQILSDRTSTSFLPDAFEDDDNSTASQKVLAAI |
| | | | | | HQFYHTQLLDYQPSDKAETINVLKSFQGLLADINNFDLDKVYLRNDKSISTIAQKMAGNYGVLR |
| | | | | | DALNYYYENKIDPEFQIKYDKATTDKKRENLDKEKSKFTRQSYISISTLQTALDTYVESFDETHDV |
| | | | | | KQVYSPTCIADYFKDHFKAEPKEGSDKEFDFVSNIEAKLSTIKGLLNTPYPENERLQQDKKKIDAI |
| | | | | | KLFLDSIMEYLHFIKPLALPEDFTLEKDEHFYTLFEEWYEQMQLLIPLYNKVRNYATQKPYSTEK |
| | | | | | FKLNFENSSFLSGWAPDYNTKGGLIIKKQDNFYLCIVEKKLKKEDVEFLKTSPEDHLAHRVIYDF |
| | | | | | QKPDNKNVPRIFIRSKGSSFPPAVHTYDLPVRSIIHIYDEGLYKTDFKKENP |
| MG29 | 6365 | MG29-42 effector | Protein | Unknown | MSNYYDSFMGIYKVQKTVRNELIPVGKTKDFIDRFVSEQNEILQADKERADAYPFVKEILDDYYR |
| effector | | | | | EFFNEVLSDFNFPEDDLQKAFILYKASIKDRSQQKDLSKFELDERNSVASALEKSKSKYALDKNK |
| | | | | | TLFNEKNGLLNSWLDRKYESGSLSKDEYEKAKKNISRFSNFSVYFTGYQQNRENMFSNEEKSTSV |
| | | | | | AFRIVNENMIRFFNNCLNFDAVCHTYPELQSKLQKYADFFVPSAFNKVISQNGEKQGIGYYNKKI |
| | | | | | LGQTEQGKESNGVNQIINLYRQKNGLKSKEVPVMAKLYRQILSVDENEKEELIEINSDQELFTVV |
| | | | | | SESAKKAASLSETLKTLLQEKMAAENFSNMFIRTDSLANLSNQLFGNWYFIKSALINAGIKKITEE |
| | | | | | YISLAALQDKLNAYIQTLDEKPDISITESLTGYFLQNMDERIKKAFESAQPSLSLQKLDADRSLPST |
| | | | | | QKPDGGKGFRQVAPIKELMDAIQEAIHLYNLFLMEFDGKEELPEEIDKDFYARFSSVFPDLKHLT |
| | | | | | KSYDMVRNYVTRKPYSTDKYKINFDRPTLLAGWDVNKEKDNLCVIFREKDQYYLGIMAAASNK |
| | | | | | LLDEESKYICPPSEEHYEKMVYKQVSGSSKMFPKVFFAKANDNLFSPSDEILEIREKGLYRKSAD |
| | | | | | DLNSLHKWIDFCKNCIAKHPEWSHYFQFHFKETNQYSNINEFYKDADDQMYNLSFINVKKSYID |
| | | | | | NAVEKGQLYLFQIYNKDRSAHSKGRENLHTIYWDNLFSEENMDRIAHSNEPVFKLNGEAEIFFRK |
| | | | | | ASLESEKPTHPANKSIQNKQQPGKALFPYDIIKDKRYTQNKLFFHCPLTINYRADDTKSKAFNTA |
| | | | | | MNKAVLADSSVKIIGIDRGERHLLYYSIIDQQGRILEQNSLNLVGNGEGETVDYHKILSDKEIQRQ |
| | | | | | NARQSWGEIEQIKDIKTGYLSQIVHRLSDLMIKNNAVIVLENLNGGFKNSRIKIEKQVYQRFEQA |
| | | | | | LIDKMNYLVFKDRSADDPGGSLHGYQLAAPFESFEKLHDQSGILYYVVPSYTSKIDPVTGFVSFL |
| | | | | | NLHYENREKSCLFIKKLTGFAYHSEADEFEIGLDYRKFGKFPGKQEWTICASNQSRYIYNHHENK |
| | | | | | YECILASDEFKKLFDSYGIEYRSGCDLRQAAASQQSADFFRQLLRLIQITWQLRYTASGASGDED |
| | | | | | DYILSPVRDEKGNFFDSRKLSDEDGGVLEPKNADANGAYHIALKGLLLLKRIQPDGTLKRVPDE |
| | | | | | KADWIDFAQNKKQLLSE |
| MG29 | 6366 | MG29-43 effector | Protein | Unknown | MEQLTNFTNLYSLSKTLRFELIPQGKTLEHIQEKGLLSQDEKRAESYKKVKKIIDEYHKEFIEKA |
| effector | | | | | LHRITLSKLYDFDFQYRLPKEQRNEDAFSKIKESLRKEIVAAFGKDETKEQFANLFKKELIKEDL |
| | | | | | LYWVGAEDKELVKEFERFTTYFTGFIIENRKNMYSADDASTAIAYRIVIIENLPKFIDNISIYENIKS |
| | | | | | NNKDLDFSPILNEMEDIIQGKTLDEIFTLDFFNNILSQNGIDFINHIIGGRSGEAGEKKSKGLNEHIN |
| | | | | | LYNQQQKDKKKRAPKFKKLYKQILSDRGSISWLPEAFEKDEEVLDAINNFYREGLENSVIDEKN |
| | | | | | VNILNEIELSFKSLLNYDDFSKIYIRNDTAITDISQTLFSDYSILGRALNYYYETFVNPKWITDYSKA |
| | | | | | TETKREKLEKERDKFTKSTYISIDILQKSLAEYIKTLDTDSEIKQKYTPTLIANYFTHHFYAKDEN |
| | | | | | GNETEKTLTYQIVSEYNGLKGFLNTEHSEDYKLIQDKERVHQLKTFLDSIMNLLHFAKPLYLDK |
| | | | | | NASEEKNELFYTEFTPIYDELAKIVPLYNMVRNYLTKKPYSTEKFKLNFENSTLLDGWDVNKEK |
| | | | | | DNTGVILLKDDNYYLAIMNKQNNTVFEEIPKAINPQNTFKKMNYKLLPGPNKMLPKVFFSKSRT |
| | | | | | KEFGVSEKMLENYENGTHKKGDNFNLSDCHQLIDFFKASIQKHEDWKQFDFNFSE |
| MG29 | 6367 | MG29-44 effector | Protein | Unknown | MPKKSLDQFTFQYSVNKTLRFALTDPQGDMEKFLANMREGELKRILVEDKQRAEDYKQVKKII |
| effector | | | | | DAYHREFIEEVLGQKGVLTEEDMNEYVTIYEEFKGLSRDSKNREKVIKKKRDIEKRLREAIVKKF |
| | | | | | KKNAKYKKLFNAKLITELLPQWLEERRNQKEIYDEAKYSEEKHLVEKFNRFATYFTDFHQNRA |
| | | | | | NMYVEKDQNTAIPYRIVNVNLPKFLDNYLNYEKLIQNHSGIDFSSIEKDLKGELRDLKLSEFMKP |
| | | | | | SNFLACLNQSGIDSYNTIVGGKTLEAGKKIQGINEILNQYRMKLDKSEAKKIPLMTSLYKQILSDR |
| | | | | | ESHSFLPEQFTSDQEMLKAIREFYESISETKEGEKKSLLNNIKEFLDSFPSENTDRIYIKATEITRIS |
| | | | | | HTLFAGDWALIHRALEYSKLDSQLKKEHIISIEEIETALQKYKEDIDEEDEAIKKKLGNPHPVIDF |
| | | | | | FKSAEKVEKVEETKNESNSNPTPYKKFNIFATINQHYQAAKEILKLEELHKDRLSPQKEGDKGG |
| | | | | | KGFQQVTKIKNLLDAIKDLLDLISPLYLEYKRQKIDVSDKDDRFYVELDILYDELFSIVPLYNKVR |
| | | | | | NHVTKKNRNEERFKINFDKTTLLDGWDVNKETANLGVILRKDNNYYLAIVHKKHSSVFNYVKK |
| | | | | | RGDSNNKLKIKDGLRRDIIAQNGEGCYEKMLYKLIKEPARDLPHAVFPEKKKNNFNPPSEEIQRI |
| | | | | | YNKYKKEKQFVNRAQMHQLIDFYKESIKRNIDWNGFNFEFSFTKEYNDIQEFYNEVKRQSYKID |
| | | | | | FDKIKSSYIEDKIKKGELFLFKIYNKDFSPHSKGSPNLHTSFWRLLFDEKNLKDTVAKLDGQAEIF |
| | | | | | FRPASIKKSERKIHKKDVPIENKNLNNAKKESKFKYDLIKDRRYTQDKFLFHVPITLNFSTQNKTA |
| | | | | | KQFNTEVNHFLQHNTNVNIIGIDRGERNLLYYTVIDQEGKILEQESLNIIANRIPNQNNIIETDYHSI |
| | | | | | LDKKEHERDRARKDWGTIENIKELKAGYLSQVVHKLTNLIIKYNAIVMLEDLNIGFKRGRFKVE |
| | | | | | KQVYQKFGELYT |
| MG29 | 6368 | MG29-45 effector | Protein | Unknown | MTMKKFVGLYPVTKTLKFELISQGKTSTHIQRKGLLSQDEQLAEQYKKVKEIIDEYHKDFIEKA |
| effector | | | | | LSGIRLTKLDDFYSQYILSKEQRDDNFFDKIKEELRKEIVAAFSKGELKIQFANMFKKELIKEDLL |
| | | | | | NWIGDEKRNSVKEFENFTTYFTGFHENRKNMYSAEEKSTAIAYRIIHENLPKFIDNIRIYDTIKFK |
| | | | | | HKNLDFSPILNELKDIIQGKSLDEIFTLDYFNNLISQNGIDFLNSIIGGRSGKSSEKKIKGLNEYINQ |
| | | | | | YNQKQNDKKDRIPKFKQLYKQILSDRSSISWMPQAFEKDTEVFDAINDFYHVELGNAEIDGRSVN |
| | | | | | ILNAVKTIVKSLSDYEELDKIYLRNDLSITTISQTIFSDYGVLGRALNHYYETFVSPQWLVDYAKA |
| | | | | | KETKRKKLEAEKEKFIKSTYISIAVLQTALAEYVKTLDDDSSIKQKYSATLIADYFTKYFYAKDEN |
| | | | | | GNEAKNTLTDQITIEYGDFKNVLDNKRSVDYKLIQDKKHVHQIKIFLDSIMNLLHFVKPLYVDKS |
| | | | | | ASEEKNELFYGEFTPVYEELAKIVPLYNKTRNYLTQKPYSIEKFKLNFENSTLLDGWDANKERDN |
| | | | | | TAVILIKDDRYYLGIMDKRHNTIFEKIPETNNRNAVFKKINYKLLPGPNKMLPKVFFSEKRMPEF |
| | | | | | GVPEEIYEKYNAGTHKKGDNFNLSDCHQLIDFFKSSIQKHEDWKRFDFKFSPTKSYKDVSGFYRE |
| | | | | | VEQQGYKITFSDVSEEYINQLVEEGKLYLFQIYNKDFSPNKKDQGKPNLHTLYWKALFAPENLA |
| | | | | | DVVYKLNGQAEMFFRKKSIDAKKTIIHKANEIIENKNPSASKKTSKFKYDIIKDRRYTVDKFQFH |
| | | | | | VPITMNFKVSGSDYINPKVNAFLRNNPNVKIIGLDRGERHLIYLTVIDLQGRIIRQESLNTIKNKQY |
| | | | | | NMETLYHELLDKREKERDAARKSWNTIETIKELKEGYISQVVHKITTMMIEHNAIVVLEDLNFG |
| | | | | | FKRGRFKVEKQVYQKLEKMLIDKLNYLVMKDNKDNEAGGLYKALQLTNKFSSFKDMGKQTGF |
| | | | | | LFYVPAWNTSKIDPVTGFVNLFNTRYENVEKAKDFFSKFDSIIFNSKEKYFEFEAKDYSKFSDKAE |
| | | | | | GTRLDWTICTHGERIETFRNSEKNNNWSHRKINITNELLKLFGTENGDFKNLIQEKTDRAFFERL |
| | | | | | LYLFKMTVQMRNSDNVEDYMISPVADKSGKFYDSRDYAKINEPSLPENADANGAYNIARKGLWI |
| | | | | | LQQINETKTEDDLKKLKLNISNKEWLQFAQRQGLVNK |
| MG29 | 6369 | MG29-46 effector | Protein | Unknown | MEEIFTEITNKNAFSLQKTLRFELKPMIFNEEKNQLQPISESDSYLKNFNSGYLEKLKQIIKHDEE |
| effector | | | | | RAEDYQEIKVYIDELHKQFIDRVLPEIKSLEIDFKKAFEMYELTKKRYAKPSSDKEEEEQSKEKK |
| | | | | | NNLKAWQDFQKEARKKISNFLKKQPEYENLFEKELFSDLIPKSNYSKQLGEKSPNDLAKSFSGFT |
| | | | | | TYFQGFHENRKNIYADEGSTSLAHRIINENLPKFFTNILQYVILNKDHNLLVGQFKENYSDEELTE |
| | | | | | LFNPNSFVSFLNQSGVDRYNEIIEAKKGIETAKSKDGLKQLANRYKQAKQIKNLPNFTPLYDQIL |
| | | | | | GKRGLDQNDESILRAGVTDDKNLLNSLKDFNKNIQPSVFELINICSELNKANAEEIFIMGSSLESLS |
| | | | | | SSVFGDYSVLSRVMKHHYIESRISASRTTEKKLEKDSEAYLKQETYSLQEIQSAIDYYIEKGNELES |
| | | | | | KSLFDYFTACKYNSNHTLSEEIKIAWDNLQPILELEQIDKDRAIPKTQEEQGGKGFQQVEKIKLF |
| | | | | | LDSYMQLLHFAKPLHLVKKRNPVTVSKKDEAFYAIFDKNYTNLEAELIPVYNQTRNYLTKKPYS |
| | | | | | LEKFKINFEKGTLLNGWDLNKEKDNLGVLFLKNNNYYLGIMSNNKIFDFQKQNIKKEALSLSGQ |
| | | | | | QDGYHKVIYKYLAGPNKMLPKVFFAKSNLEKFSASEEILRIRNTSSFTKNGEPQPSYKKAEFNLN |
| | | | | | DCHAMIDFYKQSLASHEDWSQFGFQFLETSQYSDISQFYEDVAKGGYKISFVNISDNYINEKVKA |
| | | | | | ADLFLFQIYNKDFSEQKKRKDGKPNLHTMYWNAAFRPWLDRSESNVKLNGEAEIFFREHSIERK |
| | | | | | ITHRSGEPIDRKNPKNPGESLFSYDLIKDKRFTSDKFFFHVPITINYKNKKERNNKQFNDVVNSVI |
| | | | | | KNNRDVNIIGIDRGERNLLYYTVIDQDGRILEQNSFNEISSQCSTSEKSSTFDYHKKLDEKEDERK |
| | | | | | QARKSWGTIENIKELKSGYLSHVIHKLAKLILKYNAVVCLEDLNAGFKRGRMKIEKQVYQKFEL |
| | | | | | ALIHKLNCLVLKDREEGEFGSYTNPYQLSGKITSYQDIFSQTGIVFYVNPAYTSKICPKTGFVNFL |
| | | | | | DLRYENLEKAKSLIENFNSIRFNNHENYFEFDLDYKKIPQTQNKECGEKTQWTVCTYGNERFIYN |
| | | | | | PKTRGYDTYNVTEKLAHLFKKHNISYEDGLDLREKILASTQDAVSFFKELLFLLRLTMSLRHVNE |
| | | | | | NHDCILSPIKHPELGFFDSRDVKDSTEAEEPRDADANGAYHIAMKGLQIFAEKISSENPKLSIKKE |
| | | | | | DWFRFIQNHHETKWKEKSLSPI |
| MG29 | 6370 | MG29-47 effector | Protein | Unknown | MFTNLYSTSKTLRFSLIPQGDTLNNIEKAGILAEDERLAEDFKKVKKIANDWLKNFINESLAGVSL |
| effector | | | | | SLENLLIYEEKYNLFPRNEKDEEEFDGIKTKLRKEVVSYLAKNPKFKLLGSADFIRKELPEFAKTE |
| | | | | | EEKNLINKFKTFTTYFVNYYKTRENIYSAEEKHASHAYRVINENLPLFITNKKNFDIIKNSYPELIE |
| | | | | | DIKKSVEPLLNGEKVEDMFSTEWFSKTLTQSGIDLYNKMIGGESLEDGKKIQGFNEKVNLFRQA |
| | | | | | NKLDGKSVPMLKQLKKQILGDKNVPAWITEGFKNKDSMSNAIVEFMDNIKPVLFTAADLFVAEE |
| | | | | | SHDYNKIFIKSRFLADLSHELFKDWNFLKNILLEKYTAKNPKSKNQEKEFAKISYFSVAEIQAALP |
| | | | | | NLSKDFIFEFFYNKTIKIVAEIRQSYELWNTNQENVIILKSLMDNILQLHRTFKLFDIDEADKDPVF |
| | | | | | YELFDRIFEGIDGAVKLYNKVRNFITKKPYSLEKIKLNFGNSTLLAGWDVNKESDNSSVLLRKGN |
| | | | | | DYYLAIMDKSHNKVFKNAPLVKNNEESYKKMEYKLLPKSYMMLPKVFFSGGNKHKYEPSDEIM |
| | | | | | RIYENGTFRTGDNFNINDLRKLIDFYKDSIKKNPEWSCYNFNFKPTEEYQKINEFYEDVDSQGYVI |
| | | | | | TFRDITASYIDELVKDGKVYLFKIYNKDFSVYSKGTPNLHTLYFKMLFDERNLKDTVYKLNGGA |
| | | | | | EMFYRKKFLNYSEEIMKNGHHAEELKGKFDYALIKDRRFAFDKFQFNVPITLNPNTSGHGNINDI |
| | | | | | CRDFIKSNDINVVGVHRAENHLVYITVLGSAGNIIEQHSLNEIEGYNGKNINYMEKLEKRGEERD |
| | | | | | EARVNWGVIGNIKELKEGYLSNVISKIAALMVRYNAVCAMEDLSFSFIRERSAIEKQIYQKFEKM |
| | | | | | LIDKLNFYVDKKKEPEELGGLLKPLQLANKFVSFEKMGKESGMIFYVSPYKVTDIDPVTGFVNLF |
| | | | | | DTRYFNIEKALQFFAKFKDIRYNKNTDLFEFKFDYVDFTDKDRIQSARTEWTVYTYGERIEREND |
| | | | | | NNQPKFRKIDLTKEFKNLFSEYSVDYKGDLKESILSLNEKDFFVRLLSLFRLTVQMRNGDFIISPV |
| MG29 | 6371 | MG29-48 effector | Protein | Unknown | MEIKEKTLDNFTNQYQLSKTLRFELKPVGQTAEWIKKHNIIAVDGDTLTGVDADRAKNYKYAK |
| effector | | | | | LLLDELHRLFIEDALKLAPEAESTEKLKDKIIELYSASEIKDANLPGELFKQILDDKADEWIKLYQ |
| | | | | | KEMPQYWREDISVLKDKSSRETDKKEIRNLDRIAAKLNKLCETGQSFKKTGIEILYGANEDPLKL |
| | | | | | LEWAVRCGKIRPSFKDLKQSKSDSAMPQEHIVSYIRNFDNFCTYFTGFNENRANVYDVTGAKSTS |
| | | | | | LIHRIFMQNMQFHFNNIRKWEIVRKSLEGYTNGFVEKDYNWKLKLDECEKSLSFSSDEIFTPQAF |
| | | | | | INFINQSGIDRYNEIIGGLAQEGGKTKTQGINESINLVRQHAGAKRNEFPPLQLFYKQILSKSDRTF |
| | | | | | ISAFETDEEMFDRIKDFRQKCFIEMETGKLPIIQEFIKDIDKLISESLDEKSNVFISKDKLTRISQEL |
| | | | | | TGSYNTINLRLLSELGEKVFNRNVCFSVQQIDDALNAMVDGEKFSSRNQNIKVEYQSTSGNILYD |
| | | | | | FFSKRLNTSLSSIEASWKNLNENGVFSGKELDKTRENEKEKGFEQIAAIKGFLDNSIDFLGFVKD |
| | | | | | WTLQEKKTSGNINNVWYETLQLFCDHFPIIKLYNMVRNHVTQKAHSDEKLKINFDNSTLLDGW |
| | | | | | DRNKESSNYGILLEKEGLYFLGIMTPESNSIFDYEISDSDSQTKKQGKQELANAIKASDGENLYKK |
| | | | | | VVYKQIADVSKDIFTLCWNEKENKAIRKTKGRESVWGENITRIKESKSYQNNEADRKCYFEYLIK |
| | | | | | CANSYWKHFNVKLKPADEYEDFSSLINDIDAQGYKISFDNIKKSYIDEKVFKGEFYLFQIYSKDFS |
| | | | | | QKKKSGGKDNLHTSYWKLLFDAENLKDTVLKLNGQAEVFFRKASVSLTEEKKTKGHHYERLK |
| | | | | | NKFKYPIIKDRRFAEDKFFFHCPISLNFKAEKSIPQDKYKPSFNSKFNLQIKAFLQNNSSVNIIGIDR |
| | | | | | GEKHLLYYSVTDRNGNVIEQGSLNSIAGFKGNEINYHEKLDKKEGNRDKARKSWSLIQNIKELK |
| | | | | | AGYLSQVVHKLSQLIIQHNAIVVLEDLNYRFKRGRFKVEKQVYQKFEKALIDKLNYLVFKDKKH |
| | | | | | RLEPGHCLNAYQLSGSYHLDSLKFQKQSGILFYTAASYTSTTDPVTGFMKNVYVSYESVEKSLKF |
| | | | | | WESFDSITYNPAKDRFEFKYTLGKIASKSMDKEKDEEKITKKQWTVCSCVVRSSYNQKNKTHEL |
| | | | | | HDVNQELKDLFDGKLKKLVNGKLDYLNGRDLRDSICQIQEKGFLEELVRLFNAIMAMRVIDSGK |
| | | | | | ESGTDENDFILSPVEPFFDSRKGYVGLPENGDANGAYNIARKGIC |
| MG29 | 6372 | MG29-49 effector | Protein | Unknown | MNLNQFTHLYPISKTLRFELKPVGETSDYIENFKSQYLKDIVKNDIQRASDYDIIKEIIDDYHRYYI |
| effector | | | | | EEKLSNPINTKTGEFYVNEEEIENAFSYFQKFKQNPKDDKLKKEWNDTQILLRKQLVKVFSDRK |
| | | | | | KQLFGKELITKHLPEWLQENGTWEDNKRIVENFNKFTTYFTGFHENRENMYSAEEQSTAISYRL |
| | | | | | INENLPKFFINCIQYSKIKNQFTDIEFKIDSNLLQKMGVSTIFDIFQPRYFIKLFTQTGIDNFLELLG |
| | | | | | GYTKDDGEKVKGLNEFINLYRQKNSIKSRLLPNFTALFKQILSDRETNSFIPLEFNNDNELLESLH |
| | | | | | QFIAEMGKNNGVLFKFENAVQLLQDADLSKTYIKGGINLTDISQKVFGNYGIIKIAILHYAETITYP |
| | | | | | TPKNGKISDTLMNKRKNIVNQEIFSISELESMVLQYANHLEDGHQDKEMILQFEQPILNHFLSIIK |
| | | | | | KIKQKKGEEIENIIQNVLPLLELENLSKGKAGQEQTQKIQTMLDAFLSLGHAIKPLHLVKGRKPI |
| | | | | | DIPDIDMGFYAEFSEIYEIYENLVVGLYNKTRNHLTKKPFSTDKIKINFENPTLLDGWDANKEKD |
| | | | | | NSGVLIEKNGNYYLGIMHPKYKNLFNYNKGINDLESVKRSQTKDELFDKIIDGNLNHYKKIVYKL |
| | | | | | LPGANKMLPKVFFSGGRIDFFAPSNEVLKIRNTASHSKNGAPQKGFEKAEFNLKDCHTIIDFFKES |
| | | | | | IEKHPEWREFKFNFSATSEYNDLSDFYREVAHQGYKMDFHPIKDSYINQCIQEGKLFLFQIYNKD |
| | | | | | FSPFSSGKPNLHTLYWKALFDPENIKDVVAKLNGQAEIFYRKHSIKKDERTIHKANTSLQNKNEN |
| | | | | | NPKKDSNFNYDIIKDKRYTVDKFQFHVPITLNFKAEGVTRFNDKVNHNLATQKNTHVIGIDRGER |
| | | | | | HLLYYSVINPEGKIVEQGSLNRIDTDQNYSVDYQQKLDTKEKARDKARKAWTTVDNIKDLKAGY |
| | | | | | LSHVVHKLALLIVKYNAIICLEDLNFGFKRGRFKVEKQVYQKFEKALIDKLNYLVFKDAKPNDP |
| | | | | | GYVLNALQLSAPFESFKKLGKQTGLLYYVRADYTSKIDPVTGFINFLYPKYESLAKSKIFFESFDG |
| | | | | | IRFNSSRGYFEFKIDYKKMTPSRDLIGYQTKWVACSFGDKRFKNIRNANGNWESVEVNVTEELK |
| | | | | | LLLKNENIEFKSGLDLRYDISSIKNTKFYKKLFKLLQILLSLRHSKTGTDEDFILSPIADNNGVFFD |
| | | | | | SRNANKTQPKDADANGAYNIALKGLWNIDRIKNWDGKSKLNLAMKNVEWFEFALNKPFLK |
| MG29 | 6373 | MG29-50 effector | Protein | Unknown | FDSFTTYFTGFHENRKNIYSDKAQHSSIGYRVIHENLSIFLANKRAFELVQQNFPEIAQIAQDSLLE |
| effector | | | | | HLEGGVVEDMFELDYFSLTLTQKYIDIYNTMLGGKVLEDGTKVQGLNEHINLYRQKHNIEKRK |
| | | | | | LPNlKALHKQILSDREGMSWLPDAFENRDELNSTVESFYKESIVEFACCDGVVDITEKFVETLSD |
| | | | | | DSNYDLSKIFVKNDISLTAISQEIFKDYRVIKDALWQKHLADNPKAIKSKDITADEEKYFSRKNSY |
| | | | | | FSILEIEKALDEAGQAQKLLDFFKVREIESSKTVKESFADWQNNKEDKKLTKSLLDSMLNLQRAI |
| | | | | | KPLFVKAEIDKDIAFYASFDTYFESLSGIVKLYDKVRNFESKKPYSLEKFKLNFENSTLLDGWDLN |
| | | | | | KEPDNTSVLFKKDGLFYLGIMDKKHNKIFTKISENNSKDIYQKIEYKLLPGANKMLPKVFFSNKN |
| | | | | | IDYYAPSEALLANYRDGVHKKGDNFDLDFCHELIDFFKSSIEKHPDWRNFEFSFSDTSSYEDMSGF |
| | | | | | YREVEQQGYKISYKDIDSSYIDQLVDDGKLYLFQIYNKDFSPYSKGTPNMHTLYWKMLFDETNL |
| | | | | | KDVVYKLNGQAEIFYRKKSIEYSKEKMLQGHHYDKLKDKFAYPIIKDRRFAMDKFQFHVPITLN |
| | | | | | FKAVGSDRLNDDTNELIRTNRNNIKVIGIDRGERHLLYLSLIDSSGRIVEQYSLNQIINSHNGKKHI |
| | | | | | VDYHQKLADKEKERAEARENWGVVENIKELKEGYMSHVIH |
| MG29 | 6374 | MG29-51 effector | Protein | Unknown | LDQIYIRNDVSLTNISKKIFKDYNAIKFCLETYFEKDCGKKNSEKWVSNQNYISIKDIENSIIKYFIS |
| effector | | | | | DETISSNPICDFFNSFKVHDVDLFAAISESYQVLSKDKIDLKDKLNEKDVVKIKSFLDAVMDLMHF |
| | | | | | VKPLDFNLKGKSKDKLVGAFELDAGFYDNFNKIYNGIDKDQNPNVISFVSTIVSIYNKTRNFVTK |
| | | | | | KKFSSKKFKLNFRNATLLDGWDVNKETDNYSVILLKDNDYYLGVMTKDSNKIFKSLPNCDDNDY |
| | | | | | FEKINYKLLPGPNKMLPKVFFSKKFIDYYSPSEEILNIRNCSSHTKNGNPKEGYDKKEFNLDDCH |
| | | | | | KIIDFFKKSLAKHPEWSVFNFKFKDTSKYKDISEFYKDVETAGYTLDFIKVSSKYISDLVDDGKLY |
| | | | | | LFKIWNKDFSKFSKGKPNLHTMYWKSLFSKENLENIVYKLNGQAEIFYRRKSLDKKITHPKNVPI |
| | | | | | VNKDPINNKSSSIFEYDLFKDKRYTEDKFLFYCPLTINFKSRGNDKEISKFVNQKIMNLKEDISILG |
| | | | | | IDRGERNLIYCTLIDSKGKIIGKQSMSLNGITDEFNRTSDYHKKLDDLEGKRDVARKSWTKIENIK |
| | | | | | ELKEGYLSQVIHKISKLVIDNNAIVVLEDLNFGFKRGRFKIEKQVYQKFEKMLINKFNYLVFKDK |
| | | | | | DLSKSNILKGYQLTNKFTSFQKLGHQSGILYYIPASFTSKIDPKTGFVNLLYPHYR |
| MG29 | 6375 | MG29-52 effector | Protein | Unknown | MKNFTNIYPQSKTLKFELRPYGATLDNIHKSGLIDQDETLKADYHAVKKMIDEYHKVVIDESLTN |
| effector | | | | | FKLTDLPAYEELYYKSRTEVEDKEFEIIQSNLRKQIHKAFSENKRFKSIFKKELIQKDLPAFVKKE |
| | | | | | EEREQISRFYHFTTYFTGFHENRKNIYTAEAKATSVCNRLIHENLPKFLDNRKTYLNYISNFIDLD |
| | | | | | LSQVEEDLQEVLGDITVDDLFSLDSFNHTLTQRDIDIYNLALCGRSIEGEKKIQGINECINIYRQKN |
| | | | | | RLKARQLPNIKPLYKQILSESKSGSFLLDKFEKDEDLFDSLRNFYHCLNSFNYKGEQDKSTFIELM |
| | | | | | TLFGRFSESDMTRVYLRNDASLSRLSKKLFGDWSLIVSALKYYYDAEANPLMGKKATNKYIKEK |
| | | | | | ENWLNKSSNFSIDVINKSLLRYGTINETVNSQFTDDMIFEHFSSFMIEEKNLLNTVAENYMLVSEV |
| | | | | | LSRGSLDKNQNKKKKEIKTIKTFLDTVLDLLHFIKPLSVQYVGAEKDEGFYSDFDVLYDQLSQVI |
| | | | | | PVYNKTRYYLTKKPYSMEKFKMNFKNNTLLDGWDVNKETANKGILLQKEGLFYLAIMNKDHS |
| | | | | | KSFYNIMDTGDTTGYQKMNYKLLPGPNKMLPKVFFGVKNLNFFNPSDEVLRIRNTSSHSKNGNP |
| | | | | | QEGFEKADFSLSDCHSLIDFFKASLNKHTDWKKFAFDFSPTQSYDDISEFYREVENQGYKITYTNI |
| | | | | | SDAYIHELVKEGKIYFFQIYNKDFSPFSKGKPNLHTLYWRALFDEKNLADVVYKLNGQAEVFYR |
| | | | | | KKSIEYSEEKWIQGHHHEQLKDGFAYPIIKDKRFAFDKFQFHVPITMNFKALGAPVINMKVREY |
| | | | | | LKTNPDVKIIGLDRGERHLLYLTLIDQNGNIEEQYSLNEIVNSYNGKVYKKDYQQLLHVKEGDR |
| | | | | | KKAKKNWETIEAIKELKEGYLSHVVHKIVNMMVEHNAIVVMEDLNFGFKRGRFHIEKQIYQKF |
| | | | | | EKMLIDKLNYLVLKDTQDPKTPTGLLNALQLSNKFESFQKLGKQSGFIFYLPAYLTSKIDPTTGF |
| | | | | | VNQLRIKYDSIVKSQAYYRQFDTIVYNNTSDWFEFSFRYVNFANTTPSARKIPWTICTTHHPRYA |
| | | | | | WNINSNVGNGGTEEYNVTKELKKLFDQHKIAYEEGTDLIESIASNTAVDFFKRLNKLLYITASLR |
| | | | | | HNNGKKGKEEHDFILSPVANSEGGFFNSLEADETQPENADANGAYHIALKGLWALQSIRKTDTD |
| | | | | | RLSKLNLAVSNEEWLNFAQAKQYRSQNSS |
| MG29 | 6376 | MG29-53 effector | Protein | Unknown | MKEIFNLYRQQLHSSQQEFEKKKKSFPKLAILYKQILSDCSGHSSSIEAYKTDREMLYELERLRN |
| effector | | | | | QFANKNTGCPIYNLKKLLEDIDSFDRHTIFIKSESLAEISKFLFGTWNTTRIALGEYANFLFREATK |
| | | | | | DEKIILKKQKEDFLENSGQMVFLKASDLKKYRERFIKADTFSIHDLETALYRYSQTHQDDMEGV |
| | | | | | KETGLITSYFKSFTQKINNSKVNIFNQLNEARSKIKDIVELKELSTNRNKDAKGFEQVETIKFYLDS |
| | | | | | LMNIIHFARPLHLYKGQKKIDSEGVDSEFYADFDFFYNELLDVITTYNKTRNFLTKKPYSNTKFK |
| | | | | | VNFKNPTLLAGWDVNKEKDNSGILLKKDDLYYLGIMAQGHSKNFDTGTKNSSSKNDDYQKLIY |
| | | | | | KLLPGASKMLPKVFFSDKNIDVYNPSNKVLSIRNHASHTKNGTPQKGYKKKDFNLSDCHIMINFF |
| | | | | | KSSILKHPEWVNFGFKFKDTKDYEDISEFYKEVEHQGYSVRFQSISREYIQKKIASNQLYLFQIYN |
| | | | | | KDFSPHSRGRANLHTLYWRGLFDTENSKNTCLKLNGEAEVFYRHRSIKKADQVVHKANSPVQN |
| | | | | | KNPKNPKKESQFEYDIIKDKRFTQSKLFLHVPVTLNFKAEASGKYGQFNEAVNQKLKNDLSVNII |
| | | | | | GIDRGERNLLYYTVINQRGGILEQGSLNSIKTHYKNKKNEFVEVETSYHDLLDKKEKERDLARK |
| | | | | | SWSTIENIKELKSGYLAQIVHKLAQLMIQHNAIVVLEDLNFGFKKGRFKVEKQIYQKFEKALIDK |
| | | | | | LNYLVFKDRKGGVVGSFRKAYQLTAQFQSFKKLGKQSGFLFYMPAYHTSKIDPTTGFINLVNLK |
| | | | | | YQNKEHASDFISKLESIKYNKEADHFEFDMDYKKLSDRECGPKTRWLICTHGATRYRYVPQDQK |
| | | | | | MESVDVTGQLKDLLNGAGINYMDGENLASQVLKQNDAAFFKSLLSLLNLTMTLRHSNSQKGED |
| | | | | | FILSPIRNKTGGFYDSSVVENEHESSLPQNADANGAYHIALKGAWTLRQIHDRADGAKLKLAMS |
| | | | | | NKDWFKFVQRKEYLSTPLPEEQKLKVMKGSAKGLSTPSTIGNTPQSDIQPVLKMENKK |
| MG29 | 6377 | MG29-54 effector | Protein | Unknown | MKLNKFTIIQYPISKTLRFELKPVGETADYIEDFKSQYLKDIVIQDQQRAEDYESIKVIIDEYIIRIIY |
| effector | | | | | JEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSHEEQSTAISFRL |
| | | | | | MNENLPKYFNNCIQYHKAIESYDGLSFTVNPDLLNEMGVSILFEIFQQSYYIKLFTQSGIDKFTEL |
| | | | | | LGGFTKENGEKVQGLNETINLFRQQKAIKAKAFPNFISLYKQILSDRETSSFIPDQFENDNDLLKS |
| | | | | | LGKFIKEMVKEDGLFKKLEDSIKLITDADLHRTFIKNGVEITKISQSIFGNYSILKSAIYHHAESVL |
| | | | | | YPDPISGKISEALKEKRIKYVNKQVVFSIAELETILSNYASQMTDDNPDKEIIAQCENSEHPIRTYF |
| | | | | | LNAIENVKNDKDIEFGKAIENVLPLISLENLNKGKDGQAQTHKIQKMLDAFLAVTHAVKPLHLV |
| | | | | | KGRKPIDIPDMDMGFYAEFSKAFENFEQLVITLYNKTRNHLTKKTFSTDKIKINFENPTLLDGWD |
| | | | | | ANKEKDNSGVLFEKDGNYYLGIMHPKHKNIFNYIKGINDIESEKRSLSKDELFNKIVDGESEHYQ |
| | | | | | KIVYKLLPGVNKMLPKVFFSGRRIDFFAPSTEVLKIRNSASHSKNGSPQKGFEKEDFNLKDCHTII |
| | | | | | DFFKKSIEKHPEWKEFEFEFSPTSSYEDLSGFYREVEHQGYKMDFHPIKKSYIDQCIEEGKLFLFQ |
| | | | | | IYNKDFSPYSKGKPNLHTLYWKALFDTENLKDVVAKLNGQAEIFYRKHSIKKDERTIHRANKSL |
| | | | | | QNKNENNPKKTSLFEYDIIKDRRYTVDKFQFHVPITLNFKMEKMTQFAHNEKVNQMIVKSENTH |
| | | | | | VIGIDRGERHLLYYSVINPKGHIVEQGTLNTISTDKGYEVDYQQKLDSKEKARDTARKSWTTVE |
| | | | | | NIKELKAGYLSHVIHKIAFLIEKYNAIVCLEDLNFGFKRGRFKVEKQVYQKFEKALIDKLNYLVF |
| | | | | | KDAKPNEPGHVLNAFQLTAPFDSFKKLGKQTGLLYYVQASYTSKIDPVSGFINFLYPKYESLLKS |
| | | | | | KIFFESMDGIRYNADKDYFEFSFDYRKMTPNRNLEGYQTKWTACTFGEKRFKNIRNAHGNWES |
| | | | | | VEVNVTEALKKILKNEDVDFKSGHDLRFEISKVKSTKFYKKLFKLLQITLSLRHSKTGTDEDFILS |
| | | | | | PIVDENGKFFDSRNATKDQPMDADGNGAYHIALKGLWNLEQIRNWDGESRLNLAMKNVDWFSF |
| | | | | | AYQKPFKK |
| MG29 | 6378 | MG29-55 effector | Protein | Unknown | FDYYLGISSDPKLFRSHLQDEIEKEDISDFERLDYYQLKSATVFGNSYVGDSYSKDRDLLYQKILE |
| effector | | | | | FVENTEPLKADIDKYVSSQKGTNQPTPSGIISIVKEKYPELLQKLKDDQGFSEINKTVTDRLKKTI |
| | | | | | LSLHRIPRSQEYKNHNFSLFTEAIEVIEELSSEKSFAYFKVSKSELENALNRDSKPLFLFKITNKDLS |
| | | | | | FADSFIAGKRKSRGTDNLHTLYFKALMSGSQNVFDIGTGEVFYRKEDYKGKKIVHKANEPIENK |
| | | | | | NRLNDKKHSLFEYDIVKNRRYLVDKFQFHLSIVQNYIKPKKYPDFNTEVNQAIKGASDIKVIGVD |
| | | | | | RGERHLLYLSLIDSSGRIVEQYSLNQIINSHNGKKHIVDYHQKLADKEKERAEARENWGVVENIK |
| | | | | | ELKEGYMSHVIHRIATLMVKHNAIVALEDLNFGFKRGRFKVEKQVYQKFEKMLIDKLNYLVDK |
| | | | | | QKSPNELGGLLKAFQLTNKFVSFEKLGKQSGFLFYVPAWNTSKIDPVTGFVNLLDTRYQSIEKSK |
| | | | | | EFFSKFDAIRYNDQKGYFEFEFDYKNFTTKADGTRTKWTLCTYGTRIKTFRNRDKNHQWDNVE |
| | | | | | VDLTAEFKSLFGLHSGDLKELIISQDRKEFFETLLYLLWLTLQMRNSVTNSEIDYLISPVADKNGN |
| | | | | | FYDSRVASDDLPRDADANGAYNIARKGLMIIEKIAKSKSGEKLNLTISNKEWLAYAQR |
| MG29 | 6379 | MG29-56 effector | Protein | Unknown | MDFIEECYKPIKNILLSQNEFNEEEIKNLQNFLNPFIPNRPENSDSYNGKEKNEKLKQDQYNTERG |
| effector | | | | | SVGLIGLLRLIKPVYPEKDKKKIMDLEKDASFYNEFEKLYEELSNGFPLYNKIRNYITKNSHRTEK |
| | | | | | IKINFEDSTLLDGWDVNKETDNLSVILRKKDEVTGWKYYLGVMNKHEFKGANKIFDYHIESDES |
| | | | | | DKTRQEKKKLKQTILHKEDNENFYEKMNYKFLPDPSKMLPKVFFAKKNISFFSPSSEILEIKNKK |
| | | | | | TYTKNNGDTFSLNDCHKLIDFYKASIQKHKEWGVIFNFKFSHTKKYEDISEFFAEVASQGYRLSF |
| | | | | | DRMKSSYIVDKVKKGELLLFEIYNKDFSKNSKGKTNLIITSYFKLLFEEKNLKDIVYKLNGQAEIF |
| | | | | | YRKASKQKKISHKANVPVENKNPDNPKKTSQFKYDLIKDRRFTEDKYFFHVPIDLNFKTRKTNPS |
| | | | | | KFNQNVLKFLKDNKNINIIGIDRGERHLAYYTVINQRGAILEQGSENKITANYKDQNNKNVKITT |
| | | | | | NYHALLERKEIERDQNRKAWTKIENIKELKAGYLSHLAHQISHLMIKHNAIVIFEDLNRGFKRGR |
| | | | | | MKFEKQVYQKLEKALIDKLNYLVFKEKNHEQPGGLLNAYQLTAPFESFNKLGKQTGFIFYTPSY |
| | | | | | YTSKVCPLTGFVNLIYPKYENMKKSKIYFNNFKKIYFDSKKGYFVFEYQDGKVNPSRKSESNAQ |
| | | | | | WEVCTQGPERYKYNWKARKYEKHNVTDELKKLFKEYNINYKAEEDLRESIEKQDEKKFFEGLT |
| | | | | | QALYLTLQLRHINTEEKDSDKKDFILSPVADNEGRFFDSRRVKDSEPKNADANGAYHIALKGLK |
| | | | | | MFEDIRNTKQQKLSSLKNKEWFEFIRTKPFLFDSRKAS |
| MG29 | 6380 | MG29-57 effector | Protein | Unknown | ELYTQLAPWQAYFVPENYSSILAQSAIDNYNYCVIGRLADNTDAAGVNIVINQYRQKNNIDNKIL |
| effector | | | | | HPMVQLQKQILSDKPSFVPVIETNEEAVSLVKVNYCKLQELNDKLQALISEQITPETIGDFYFRSI |
| | | | | | KLTGISLDVYLDWNYINQAKDIYIQFLNEKQQKAFYEKEWGKVINISTLIRIITGFKRVRIGESSNV |
| | | | | | SEESTIKELLTYVNAAPDFTVDIEKISANNIANYKDVLDKMNANIQFYKMFSLYDGNKKINVTNK |
| | | | | | KDYFYNAFDSIYSEMKAISRDYDKIRNFATKRTKGENKVKLNFANAQLLSGWDMNTEKNYHSVL |
| | | | | | FAKDNKFYLGILGNKASGVFNFSNPDIVLKANEPGQKYQKMMYKQMGGANKMLPKYAKDLAT |
| | | | | | KHALPKHIQEIYEKKLYTKEANNPNAMYIWIEWCKEALKTNPNWANYYSFNLLDSQQYKNMNE |
| | | | | | FYTAVDNQAYFVEWADVSEAYIDELIDSGKLYFFQIYNKDFSDKKSKPGTDDLHTLYWKALFSPE |
| | | | | | NLSRKEGPIFKLCGNGEIFWRQPSVEYHVTHRANQPLQNKNPFNPKRESVFSYDLVKNKRFTEN |
| | | | | | KYFFHVPIKLNFRSGNTVFKYNEKVNRYVEGNKDINIISIDRGERHLLYYTVIDQQGKILEQNSLN |
| | | | | | VIYNTHLIENNTIWTKINYRDLLDKKEKQRADARLAWENIESIKELKTGYLSQVVHQVTKLMEK |
| | | | | | YNAVVVMENLNTK |
| MG29 | 6381 | MG29-58 effector | Protein | Unknown | MNFNNFTHQYSLSKTLRFELKPIGETADYIEDFKSQYLKDIVTQDQQRAEDYKVLKEIIDDYHRH |
| effector | | | | | YIEEKLSEPFDKKTGELFISEEDFENAFSYYQRFKEDPKDEKSKKDWLDTQSSLRKSVVKIFSDRK |
| | | | | | KRLFQKELITKELPAWLKEKGEWEEKKNVVENFNRFTTYFTGFNENRENMYSDEEKSTAISFRL |
| | | | | | MNENLPKFLNNCLQFGVIIEKHIDLDLKIESKLLQKMGVSNLNEVFQPSYFIQLFTQTGIDNYSEL |
| | | | | | LGGWTKENGEKIQGLNELINLHRQKYSIKAKGLPNFIGLYKQILSDRESSSFIPDQFENDKALLES |
| | | | | | LKIFVKEMAKTDGIFVNLKKAVTLLKEADLDKTFIKGGVEISKISQAIFGQYSIIRSAIYNYAETV |
| | | | | | MYPTPKTGKVSDVLEEKRKKYANNEDIFSIAELESMLTSYREQLEDEHPDKEIITHCENSEHPIRT |
| | | | | | YFLNIIDNVKNDKDIELGKSIENVLPLLSLENLNKGKAGQTQTHLIQKMLDAFLAVTHAVKPLHL |
| | | | | | VKGRKPIEVPDIDMGFYADFSSAFEIYQQMIIGLYNKTRNHLTKKPFSTDKIKINFENPTLLDGWD |
| | | | | | ANKENDNSGILFKKDGNYFLGIMHPKHKNIFNYIKGINDIESEKRSLSKDELFDKIVDGNSDHYQK |
| | | | | | IVYKLLPGVNKMLPKVFFSGRRIDYFAPSTEVLKIRNTASHSKNGKPQKGFEKADFNLKDCHTII |
| | | | | | DFFKQSLEKHPEWKEFEFDFSPTSSYEDLSGFYREVEHQGYKMDFHPIKKSYIDQCIEDGKLFLF |
| | | | | | QIYNKDFSPYSKGKPNLHTLYWKALFDPENLKNVVAKLNGQAEIFYRKHSIIKDDRTIHRSNKSL |
| | | | | | QNKNENNPKKTSLFEYDIIKDKRYTVDKFQFHVPITLNFKAEGVTRENDKINRELSKNSIQTFAW |
| | | | | | SK |
| MG29 | 6382 | MG29-59 effector | Protein | Unknown | MLSHFTNQYQLSKTLRFELKPVGETLKHIELKGLLAQDETRSQEYQEIKIIIDKYHKAFIEEALHD |
| effector | | | | | VSLSKLAEYETAFFDKNRDEKAFEKLQDALRKEIVMQFKKDAIIYATLFKKELIKADLQNWQEL |
| | | | | | SDAEKELVSHFDNFTTYFTGFHENRANMYTDEAKHSSIAYRIIHENLPIFLINKKLFEIISQKAPQL |
| | | | | | AQETQDALLEHLNGAIVADMFELSYFNRLLSQSHIDLYNQMIGGVKKDELKIQGFNEKINLYRQ |
| | | | | | ANGLSKRDLPNLKPLHKQILSDRETLSWLPEAFENYEELVQGVQAYFNSEVLTFECCDGKVNLL |
| | | | | | EKLPELLNQIQDYDLSKIYFKNDVALTSASQAIFKDYRIIKEALWEVNKPQKSKDLAADEEKFFN |
| | | | | | KKNSYFSIEQIDDALKEAKLFASMMAYFQSESSKTIEQIQVAYAQWKQNTSNKELLKAFLEVLLS |
| | | | | | YQRLLKPLNAPNDLDKDVAFYVYFDTYFASLSGIVKLYDKVRNFMTRKPYSLEKFKLNFENKGD |
| | | | | | FLGGWVDSHTENSDNGTQAGGYLFRKKNHIGEYDYYLGISKDTKLFRSHLQNEIQEEDKSEFER |
| | | | | | LDYYQLKSASVYGNSYIGGSYDEDKKMLLKSIYKFAENNHPLKADFDKYIASQKGDNQPTPNGM |
| | | | | | INIVVEKYPQLMQELVKNEEFEAANKMVIERLKQTILSLHRVPKSQDYRDKVFSLFIEPIQIIEELS |
| | | | | | KEKSFFYFHVSDNEFKSALARDEKPLLLFKITNKDLSFADSFASGKRKSRGTENLHTLYFKALMS |
| | | | | | GNQNVYDIGTGEIFFRKKSIEYSEERLKKGHHFDDEKKLKDKFAYPIIKDRRFAFDKFQFHMSIIQ |
| | | | | | NYMYPKQPKGFEEKVNLAIHDADNIKVIGIDRGERHLLYLSLIDSNGKIIFQETLNSIVNTFNNEPI |
| | | | | | DYHSKLDAKEKDRDKARKEWGVVENIKELKEGYLSHVVHKIATLIVEHGAVVAMEDLNFGFKR |
| | | | | | GRFKVEKQVYQKFEKALIDKLNYLVDKKKQPDELGGILNALQLTNKFQSFEKMGKQNGFLFYV |
| | | | | | PAWNTSKIDPVTGFVDLFDTRYASVEKSKAFFAKFKTIRYNEAKDYFEFAFDYNDFTDKAKDTRS |
| | | | | | EWTLCTYGERIVSFRNAEKNHQWDSKTLHLTTEFKNLFGSYTGDLKATILAHDDKEFFEKLLRL |
| | | | | | LRFTLQMRNSITGTDIDYLVSPVADEQGNFYDSRKADASLPKDADANGAYNIARKGLMLMRRIQ |
| | | | | | KAEDPKKVNLAISNRDWLRCAQGLEK |
| MG29 | 6383 | MG29-60 effector | Protein | Unknown | MQKNDTVLKFNKHSNKLLCSFTRKYPLSKTLRFELKPVDDTKKYLKDFVQSDQERAGDYKELK |
| effector | | | | | KIIDEYHKDYIEKSLSKNDILSLDDLNNLKEHIKKSGSLQPLEEKQKNEKSIKKELHTLNKTTKSK |
| | | | | | PKKNEESIIKKLQNTLRKQITQAFQLKENKDRLFGKKLINEVLPEWLDSCSLEDIEHKKEILKKF |
| | | | | | QKFTTYLTGFHENRKNIYSDKEQSVAVPHRIINENLPKFLSNLNTYEKIEKHFPGLKTHFESLKEQ |
| | | | | | LKAEFEYFEIQNIKDLFKINLFNRCLTQEGIDNYNAIIGGRVLGSGQKIRGINEKINRFRQIQPEKG |
| | | | | | DRETIRIISNKNLPIMQTLYKQILSDRESHSFYFEEFKSRKEVLDSVNLYWESIFKKRGNQSVVQRI |
| | | | | | KNLFTNLKKYELDKIYFKSSDLSHVSNKLFGDYSVIGSALNFHAEKKFSKKKEREQYVEKDFFSF |
| | | | | | DEIHKALSGYLKENKEIQVSKHFSGFKEIHKALSGDSQTGQNNILAVYFNWEFSKKKYNNKSLLD |
| | | | | | FIEECYKPIKNILLSQNEFNEEEIKNLQNFLNPFIPNRPENSDSYNGKEKNEKLKQDQYNTERGSV |
| | | | | | GLIGLLRLIKPVYPEKDKKKIMDLEKDASFYNEFEKLYEELSNGFPLYNKIRNYITKNSHRTEKIK |
| | | | | | INFEDSTLLDGWDVNKETDNLSVILRKKDEVTGWKYYLGVMNKHEFKGANKIFDYHIESDESDK |
| | | | | | TRQEKKKLKQTILHKEDNENFYEKMNYKFLPDPSKMLPKVFFAKKNISFFSPSSEILEIKNKKTY |
| | | | | | TKNNGDTFSLNDCHKLIDFYKASIQKHKEWGVIFNFKFSHTKKYEDISEFFAEVASQGYRLSFDRI |
| | | | | | KSSYIVDKVKKGEFLLFEIYNKDFSVASKGKPNLHTSYFKLLFEEKNLKDIVYKLNGQAEIFYRK |
| | | | | | ASKQKKISHKANVPVENKNPDNPKKTSQFKYDLIKDRRFTEDKYFFHVPIDLNFKTRKTNPSKEN |
| | | | | | QDVLKFLKDNKNINIIGIDRGERHLAYYTVINQRGAILEQGSENKITANYKDQNNKNVKITTNYH |
| | | | | | ALLERKEIERDQNRKAWTKIENIKELKAGYLSHLAHQISHLMIKHNAIVIFEDLNRGFKRGRMK |
| | | | | | FEKQVYQKLEKALIDKLNYLVFKEKNHEQPGGLLNAYQLTAPFESFNKLRKQTGFIFYTPSYYTS |
| | | | | | KVCPLTGFVNLIYPKYENMKKSKIYF |
| MG29 | 6384 | MG29-61 effector | Protein | Unknown | VEKDLKDILNSSNLKEFLSLENFNNCLNQKGIDKFNLLIGGKSEEGNKKIQGLNERINLFSQKLEK |
| effector | | | | | KESHLFRKLKMVPLFKQILSDRVYSSFVLEKFKDKKELFETINCFYNGSTSEGGDEFEGFKKISIK |
| | | | | | IKNLITTLKENDLDHVYVKKDSLSKISQELFGDYNKLNECLGEKFSKGLEGKLNTKGKLFTPAQI |
| | | | | | KKEVEKLLKNKFFSLKEIEDAIHFLDEEEQNKSVINDYFLLFKQKGLHESDLFKQIDQNYYEYSKI |
| | | | | | DKNSPYKLNERKSELDVEVIKKLLDAILDFYHFIKPFHTKIKKDDNDKGADALEKDAEFYGSFDS |
| | | | | | AFEDLEKIIPLYNKVRNFVTQKPFSTKKFKLNFDNSTFAAGWDVNKEKDNSTVILRKFNSKRNDF |
| | | | | | DYFLGIISGIDKKIFEKTSLVSTKSSFYEKLRYKQIADVSKDIQNLMVINGKTVSKKGRKNTQGIN |
| | | | | | QQLEDLKNKYLPKEINLIRKKKSYLKNESTFSKKDLALFIDYYKKRLDYWDFKFELKKSEEYTD |
| | | | | | FNDFTTHLGSQGYKLSFISLSEEYINNLINEGKLYLFQIWNKDFSEYSKGRPNLHTIYWRELFSEE |
| | | | | | NLRNIVYKLNGEAELFYRRASIKEKIFHKRGVKIAKKWFKDKPVPKEIVLKLNRYYEGKISKDDL |
| | | | | | NNDDLKFINNFVIKEQEEGKNDVIIKDKRYTEDKFLFHCPITLNFKAKGSEKNVHKLINKHINETR |
| | | | | | DEINIIGIDRGERNLAYYSLINSKGEILEQNSFNIISDDLQRKLDYQEKLDQIEGDRDKARKNWKKI |
| | | | | | ANIKEIKEGYLSQVIHKIAALAIEKNAIIVLENLNFGFKRGRFKIEKQVYQKFEKMLIDKLNYLVF |
| | | | | | KSNASEDKGGSLNAYQLTNKFETFQKLGTQSGILYYVDAYKTSKICPRTGFINLLFPKFETIKQSK |
| | | | | | EFFSKFNFIKYCSKEDLFEFNFDYSSFSGDKKTIYHKLVKDNWSVWSNGIKLINKRDKNSNNKWK |
| | | | | | TFEFDANKELKSLFEEYHIDYSSQENLINSILAVDNKSFFELLIFNLKSILQLRNSYLDFEVKSFKEK |
| | | | | | EGESYKESNYDYILSCVKDKNGLLFDSRKAKNFEIQDADANGAYHIALKGLMLIEKIKEEPDYNN |
| | | | | | KTKIDLKIDRTKFVNYVIKKNS |
| MG29 | 6385 | MG29-62 effector | Protein | Unknown | MKINKFNDFKNLYQLSKTLRFELIPQGKTLEHIEKNGLLQQDEQRAKDYQQVKKIIDDYHKHFI |
| effector | | | | | DEALANTKLENLQQYYDLYIKRDKSDDDKKQLENIKKELRIRLIEAVKSNTKFTTIFKKELIKDD |
| | | | | | LVAWLEKQAREQENEVNLIKKFSDFTTYFSGFHENRKNMYSDEEKSTSIAFRLIHQNLPKFVDNL |
| | | | | | QIFEKVKSSGLDFSSMSLLLGDVPLEDFFKLEYFNQTLTQVGIGLYNLMLGGQSSDDGFKLQGLN |
| | | | | | ELINLYNQRQDDKKNRLPKLKPLFKQILSDRDGYSFVLESFKNDGDVCNALSDYYVKKLSNSECI |
| | | | | | FERLPTIFNHINEYDTKQIYINKSALTNLSAKLYGSWDVISKALEFYYITKIDNDSNKNETAIKKKE |
| | | | | | KWLKQEYFTLCDLDEVIKFYVDNQQIDKCAVTQYLVNYFAQIFTDPNNLIAQITSQYNAVQALIQ |
| | | | | | NIQPDTKNLIQEKENVAEIKKLLDLIMELLHVCKLFVIKSDIAEKEHNFYSELEYCLALLNPVIAL |
| | | | | | YNQIRNYLTQKPYSLEKFKLNFENSTLLDGWDRNKEPSNTSILLRKNGNYYLGIMNKRHNRVFE |
| | | | | | VLPQILETNNSYEKVVYKLLPGANKMLPKVFLSTKGKSTFQPSQELLDNYKSETHKKGDNFNLD |
| | | | | | HCHKLIDYFKDSINKHSDWKKFNFNFSDTATYEDISRFYREVESQGYKITFDLISEQYINELVNDG |
| | | | | | KLYLFQIYNKDFSPHSKGTPNMHTMYWKALFSEENLANIVYKLNGQAEIFYRERSITKDKIIKHA |
| | | | | | ANQPINNKNIDNDKKQSTFAYDLIKDKRFTMDKFQFHVPITLNFKAQGRDDINLEVKEYLKTNPD |
| | | | | | THVIGIDRGERHLLYVSVVNPQGEIIKQFSLNEIINEYKGNTYKVDYHNLLNNKEGDRKKAREN |
| | | | | | WGVVENIKELKEGYLSQVIHKICQLVIEYQAIIIMEDLNSGFKNSRIKVEKQVYQKFEKMLIDKL |
| | | | | | QYLAFKNPRGNQPDIYNALQLASKFDSFTELEKRKQSGFIFYVPAWNTSKIDPATGFVDLLKPKY |
| | | | | | ETVAKAQEFIRKFDEIKYNQNKDWFEFSFNYSKFTEKVDGIRLNWTVCTTNVNRYSWNRKLNN |
| | | | | | GKGEQQLFEISKCLKALFDIYKIDYQTSENLIEQIARQSEKDFYVCLMRYLSITLNLRHNNGKSGI |
| | | | | | DEEDYIASPVADNSGAFFDSRVEVGKGKDKNGNWVSKLPVDADANGAYHIAKKGLWVLQQLK |
| | | | | | KADDLRKVKLAISNKEWLEFTQN |
| MG29 | 6386 | MG29-63 effector | Protein | Unknown | MLSNFTNQYQLSKTLRFELKPIGNTLEHIEQKGLLSQDEQRAENYTVIKEVIDTYHKAFIEESLAS |
| effector | | | | | VVFDNLERFEELYLKSNKDEKEQKEFEKLQENLRKEIVKNFKVHPKWNNLFKKELIKEDLLAFE |
| | | | | | QITDEQKEVVKEFTNFTTYFTGFHENRANMYTDKEQHSSIAYRIVHDNLPTFVNNKKAFESILQK |
| | | | | | YPQLISDAKSSIEEELLGAVFEDMFLLQYFNHLPSQTHIDLYNTMLGGVKRDDLKIQGFNEKINL |
| | | | | | YRQANGLNKKELPNLKPLYKQILSDKDTLSWLPEAFETQEELVGAVESFYQEKILAFECCDGRV |
| | | | | | NLLEKFKEIFSQTQLYDTSKIFIKSDKPLTDISQALFKNYGLLKEALWQKHLDDNPKLQKSKKIE |
| | | | | | ESEEKFFKQKYFTLSSLQEAIEFAKLSANVWNYFQENLDTYIKQIEENHTIWETDKTNTATTKSF |
| | | | | | LDSLINLQRFLKPLNVQTDSDKDIAFYSTFDSYFEALTQIVKLYDKVRNFKTKKPYSLEKFKLNFE |
| | | | | | NSTLLDGWDVNKEPDNTSILLRKNNLYYLAIMDKKYNKLFCNLEKSTQSDVYEKIEYKLLPGAN |
| | | | | | KMLPKVFFSNKNIDYYNPSKKLLENYKDGIHKKGDNFDIDFCHELIDFFKVSIQKHEDWKHFKF |
| | | | | | NFSPTKSYEDLSGFYREVEQQGYKISYKNIDTKLVESWVNDGKLYLFQIYNKDFSPYSKGTPNMH |
| | | | | | TLYWKALFDEQNLANVVYKLNGQAEIFYRKKSIEYTEDKLKKGHHHEELKDKFAYPIIKDRRFA |
| | | | | | FDKFQFHVPITLNFKAEGNENLNQKTIEYIKTNDIKIIGIDRGERHLLYLSLIDLNGRIVEQYSLNQ |
| | | | | | IINSYNGKEYPIDYHEKLAKKEDERALAREEWGVIENIKELKEGYMSHVIHSITTLMVEHNAIVV |
| | | | | | LEDLNFGFKQGRFKVEKQVYQKFEKALIDKLNYLVDKKKTPSDLGGVLNALQLTNKFVSFEKM |
| | | | | | GKQNGFLFYVPAWNTSKIDPVTGFVNLFDTRCSSVEKAKEFFGKFKSIRYNSVKEYFEFEFDYND |
| | | | | | FHNKALDTQTQWTICTYGERIKTFRNKDKNSQWDNETIHLTTAFKNHFGNYQGELKEYILVQD |
| | | | | | KKEFFKQLLDLFKLTLQMRNSITNSEVDYLISPVADKNGNFYDSRKADSSLPKDADANGAYNIAR |
| | | | | | KGLMLVERIKESTDVKKVDFKLTNKEWLQFAQRG |
| MG29 | 6387 | MG29-64 effector | Protein | Unknown | FGEFNTIKVDLRNEFIRNNNYNVDKLNKTQEKEIEKYLKKEYFTFQEIEDSILRIKNSKDDSSDIKS |
| effector | | | | | LFGYFKDFEFSVEHKKISLLETIEEKYKSFKSINFRDFEDSTQKKLTQNDYVEVVNTIKEFLDLLL |
| | | | | | HYYHFVKVLHYTGEDRDEHFYKEYDELLESISQITSLYNKVRNYISQKPFSTEKFKLNFEKSTLA |
| | | | | | DGWDLNKERQNLSVIFRKNSNYYLGIISKEDTKIFADIEEDLSGEYFEKMEYKLLPGASKMLPKV |
| | | | | | FFSKSNIDYYKPNDEVLRIRNTSSHTKGGDPQKGFSKFDFNIDDCRKMIDFYIISLNKHPEWKDEN |
| | | | | | FKFRKLDEYGSIDEFYREVEEQGFKLNFKKISQDYIKQLVDENKLYLFQIYNKDFSENKTKKENY |
| | | | | | SSKKNLHTMYFEELFSKENLEDVVFKLNGQAEIFYREKSMEYKPTHPKNLSTLNKDPINGKKESI |
| | | | | | FSYDIGKNKRYTQDKVLFHVPITLNFKSNSRVKINNEVNKIIQKNHNFVNILSLDRGERHLLYYTL |
| | | | | | LDNKGNILDKGTFNLVNDKFDRNVDYHQKLSVLEKERDEKRKSWQNISTIKELKEGFLSQIIHKI |
| | | | | | SKIAVDNNAVIVLEDLNYGFKKGRFKVEKQVYEKFEKMLISKLNFLMFKDKHNNEIGGSLKAYQ |
| | | | | | LAPQVNVLKDIGKQTGILFYVDPYLTSKICPKTSFVNRLHPKYENEKQARDFFKKFDSIKYLEDE |
| | | | | | DLFEFSFNYSSFGVKGLVKDDWKIYSNGIKLVQSRDKSQNNNWVTKEVNVNEELKKLFNDFNIEI |
| | | | | | SNSTNLIDDITNQNKFFLESLIRNLKLILQLRNSYTDNELNVKKISEKDGDYILSCVKDKEGNFFDS |
| | | | | | RKAQDNDVDNADCNGAYHIGMKGLMVLNKIKEFEDIEKLKFNDLKIERQEFLNEMIKRNWG |
| MG29 | 6388 | MG29-65 effector | Protein | Unknown | MFTNLYPTSKTLRFSLIPQGDTLNNIEKAGILTEDEKLAEDFKKVKKIADAWLKNFINESLTGVSL |
| effector | | | | | SLENLLIYEEKYNLFPRNEKDEEEFDGIKAKLRKEVVSYLAKNHKFKLLGSADFIRKELPEFAKT |
| | | | | | EEEKNLINKFKTFTTYFVNYYKTRENIYSAEEKIIASIIAYRVIIIENLPLFIINKKNFNAIKNSYPELI |
| | | | | | EDIKKSVEPLLNGEKVENMFSTEWFSKTLTQSGIDLYNKMIGGESLEDGKKIQGFNENVNLFRQ |
| | | | | | ANKLDGKSVPMIKQLRKQILGDKNTPEWITEGFKDKDSMNEAIVKFIKNIEHANDNLTGELFIRS |
| | | | | | KPYDYNKIFIKNRFITNISHELFKDWNLLKNNMLEQYKSKNPKSKNPEKEFAKIPYFSIAEIQESIP |
| | | | | | NQNKHFPDFIIDHFHDKIMTLIPTEKKIHELWKDNKDSIPALKNLMDYYLELYRISKPFDADCAD |
| | | | | | KDPVFYELFDEIFNDFSKVVKLYNEVRNFITKKPYSVEKIKLNFGNSTLLAGWDVNKESDNSSVL |
| | | | | | LRKGNDYYLAIMDKSHNKVFKNAPLVKNNEESYKKMEYKLLPKSYMMLPKVFFSRSNKHTYEP |
| | | | | | SDEIMRIYENGTFKAGDNFNADDLHALIDFYKDSIKKNPEWSCYNFNFRPTEEYQKINEFYNDVD |
| | | | | | SQGYDITFRNIKASYIDELVRDGKIYLFKIYNKDFSTYSKGTPNLHTLYFKMLFDERNLKDTVYK |
| | | | | | LNGGAEMFYRKKSLNYPEEIMKNGHHAEELKDKFDYAIIKDRRFAFDKFQFNVPITLNPNAQDR |
| | | | | | GNINDICKNFIKSNDINVIGVHRAENHLVYITVLDSAGKIIEQHSFNEIEGYNDKTINYMEKLAARS |
| | | | | | NERDEARVNWGVIGNIKELKEGYLSNVISKIAALMVKYNAVCVMEDLSYDFIRERSAIEKQIYQK |
| | | | | | FEKMLIDKLNFYVDKKKEPEELGGLLKPLQLANKFVSFERIGKESGMIFYVSPYKVTDIDPVTGF |
| | | | | | VNLFDTRYFNVNKSKDFFGKFKNIRYNEKTNLFEFTFNYDNFTDKDKIQSARTEWTVYTYGERIE |
| | | | | | RFNDNNQPKFRKIDLTKEFKNLFSDYSVDYKGDLKESILSLNEKDFFVRLLSLFRLTVQMRNGDF |
| | | | | | IISPVMDKTGKFFDSRKPNGKALPENAAANGAYNIARKGLILLNRIKQSENIRKVDLRLSGDEWL |
| | | | | | QFAQGGDL |
| MG29 | 6389 | MG29-66 effector | Protein | Unknown | MKDFNQFTNLYSLSKTLKFELKPIGKTLDYITEKSLLNEDEQRAESYKRVKKIIDEYHKKFIDSCL |
| effector | | | | | RGVKIGNLQEFESLYFSSNKDEKELTSLQKKMRKEIADRFTKTDAYKRLFGKELIKEDLAQIVQD |
| | | | | | ESILKDIEDFKNFTTYFTGFHENRKNMYSADEKATALAYRLIHENLPRFLSNKRIFEKITKEHPQII |
| | | | | | EQAKKSVEPHLFGISIADMFNPEYFNQTLRQEDIDIYNLMLGGKSDSNTKIQGLNESINLYRQRK |
| | | | | | GLTKRDIPNLTVLYKQILSDRESFSSVLEKFEKPQDLLEAVKEFYINRLTKWADGDNVYNVLEQL |
| | | | | | KFIVSDDGQYDTSKIFIKNDTSLTDISQQLFGSWSVISNALKQDYYLSNSKLKPSDENDAKFEKIKF |
| | | | | | YSIQKIEDVLKRYCAEMDDLKEKYTENIILKYFASFRRKDDTLTLIQKAENYYVEAADLLNAEYP |
| | | | | | PNKSLISDDDAIEKIKNLLDAVMDIIHFVKPLRAKGYEGEKDEAFYAEFLKFADQLDKLIPLYNKV |
| | | | | | RNYLTQKPYSVEKFKLNFENSTLLDGWDQNKESANTSVLFAKNGLFFLGVMDKKHNKLFEKTP |
| | | | | | PHKTEDCFEKINYKLLPGASKMLPKVFFSAKNITYFAPDENVIRIRNHGTHTKNGEPQKGFEKKD |
| | | | | | FSISDCRIMIDFFKSSIEKHPEWKNFKFKFNQTSSYNSIDEFYRQVENQGYNISYQKIDKEYIDELV |
| | | | | | SQGKLYLFQIYNKDFSEHSKGKPNLHTLYWKALFSPDNLKDVIYKLNGQAEIFFRKKSLQYSDET |
| | | | | | LKRGHHADKLKDKFAYPIISNKRFAFDKFQFHVPITMNFKASGRDNINQEVIEFLKSVPENEIHFI |
| | | | | | GLDRGERHLLYLSMINSKGEILKQFTLNDIVNEYNNQKYTTDYHKLLAEKEKNRAESRTQWKTI |
| | | | | | ETIKELKEGYISQVVHIVAKMMVENRAILLLEDLNFGFKRGRFKVEKQVYQKFEKMLIDKLNYY |
| | | | | | VDKSKSPTEPGGVLNALQLASKFTSFKDLGKQSGFIFYVPAWNTSKIDPVTGFVNLFNTKYENVA |
| | | | | | NSQKFFEKFQDIRWNEAQNHIEFHVKNYTDFNPKAEGTRQNWIICSHGTRLENFRNEKQNSRWE |
| | | | | | TREVPLTEEFLKHFHDHNIKPQTGLKEQIVARQDKQFFERLHHLLKLTLQMRNSSKETGEDYLI |
| | | | | | SPVADEKGVFFDSRHFAKQEISEMPKDADANGAYNIARKGLLLLNKIKESKSDSKVDLKQTNKD |
| | | | | | WLNFVQKSTLK |
| MG29 | 6390 | MG29-67 effector | Protein | Unknown | NSYFLAIMDKQNNRIFENVPKIRNNQVVFKKVNYKLLPGANKMLPKVFFSKSRIKEFGVSAKML |
| effector | | | | | ENYKAGTHTKGNNFDISDCHQLIDFFKSAIQKHDDWKQFDFNFSKTESYEDTSGFYREVEQQGY |
| | | | | | KITFSDVPEEYIYQLVEEGKLYLFQIYNKDFSEHSKGKPNLHTIYWRSLFSPENLSNVIYKLNGQA |
| | | | | | EIFYRKKSINPEKATVHKANLELENKKCRDGLLSVFTTPEPKKYSIFCYDIIKDRRYTIDKFQFHV |
| | | | | | PITMNFKSTGNDYINPIVNEYLRNNKDIKIIGLDRGERHLIYLTLIDLQGNIILQQTLNTIKDDQHDI |
| | | | | | ETPYHQLLDKKETERDKARKSWDTIENIKELKAGYISQVIHKIAQMMYQYNAIVVLEDLNFGFK |
| | | | | | QGRFKVEKQIYQKLEKMLIDKLNYLVVKENDANQPGGIYKALQLTNKFSSFKDMGKQTGFLFY |
| | | | | | VPAWNTSKIDPVTGFVDFLKPKYESIEQAKLFLSKFKKICFNTAKNYFEFSFDYSDFTTKAAETRT |
| | | | | | NWTICTFGDRIETFRNPEKQNKWDNRDVVLVKEFTGLFDKYNLNYKNGTDIKEQISKQTEKGFF |
| | | | | | EKLLYLLRLTLQMRNSKTGSDIDYLISPVADKNGNFYDSRNVETDKYLSLPEKKKNVQVVETYN |
| | | | | | YTPLPQNADANGAYNIARKGLWIIEQINKTDNLKKINLAISNKEWLQFVQK |
| MG29 | 6391 | MG29-68 effector | Protein | Unknown | MKSDFFKNFTSQYKLSKTLRFELKPIGKTLESIQSKGLLEKDEQRASSYKRVKKIIDEYHKYYIEL |
| effector | | | | | ALKNIQLSKLAEYYELFSQNKEVRNEDAFKNLKNELRKEIVKELTKGAYKEMFERLFSKELIKE |
| | | | | | DLKLWIKDRPEFQEELQFIKEFDNFTTYFTGFNENRRNMYTDQEQSTAIAYRIVHENLPKFIENIK |
| | | | | | MYESIKLKYPDLNFTPVLKDMEDLIQGKTLDEIFSLEYFNNVLSQHGIEFLNFIIGGRTREDGTKI |
| | | | | | KGLNEYINLYNQQQMEKNKRSPKFKQLYKQILSDRTSISLRFEAFENDSELLDAIEEFYQTELCE |
| | | | | | YESEGKTKNVFDEIKNLVTSIEAYDLEKIYLRNDTNLTNISQRIFGAFGVFKDAVSYYYDHVIDPQ |
| | | | | | FQTKIAKAKGEKALKKLNDLKAKWNKEYISIAVLQTALDKYMESVDDSLEIKKTYTLKVIADYF |
| | | | | | KNHFKVIDEEKKETDLVYNIKSQYLGVKGLLNIEKSENKILAQDKEKVHQLKSFLDSILELNHFV |
| | | | | | KPLNLIADALLEKDEVFYSQFAPLYEQLNKLTPLYNMVRNYLTQKPFSTDKIKLNFENSTLLDG |
| | | | | | WDVNKEPDNSSVILRKEGLYYLAIMDKDNKRIFLNVPGIENETGFYEKMNYKLLPGANKMLPKV |
| | | | | | FFAKSRIDFFNPTQEILENYKNETHKKGDTFNIDDCRALIDFFKSSLEKHEDWKQFNFKFSPTNTY |
| | | | | | QDLSGFYREVEHQGYKMSFENIAADYINKLVEEGKLYLFQIYNKDFSTFSKGKPNLHTMYWKAL |
| | | | | | FDEKNLANVVYKLNGEAEVFFRKSSIEEKNKVVHKANESIISKNPLTKGKLNTFEYDLIKDRRYT |
| | | | | | VDKFQFHVPITMNFKATGSEFINFQTNEFLKNNPEVKIIGLDRGERNLIYLTLIDQKGNILIQESLN |
| | | | | | TIKNKVRDIEINTPYQELLNKKEKERDEARKSWGTIENIKELKEGYISQVVNKIATMMVEHNAIV |
| | | | | | IMEDLNFGFKRGRFKVEKQVYQKLEKMLIDKLNFLVFKDKPSTDTGGLLNALQLTNKFESFQK |
| | | | | | MGKQSGFLFYVPAWNTSKIDPVTGFVDFLKPKYENIEQSQKFFSRFDMIKYNSSKDHFEFTFDYK |
| | | | | | NFTEKADGTRTQWTVCTAGNERYYYNPADKITSKINITEKMKELFEKENFNYKNGENIKQQITN |
| | | | | | SESKYFFIMLSKYLGITLALRYSNSADGRDFILSPVTNEKDEFYHSETADKLLPRDADANGAYHIA |
| | | | | | LKGLWVLQQLAKQEDLKKIKLAIGNKEWLSFVQNKNY |
| MG29 | 6392 | MG29-69 effector | Protein | Unknown | LLHFLKPLYLKTDAEVQRDDLFCSTFTPLYEQLGSITKLYDKVRNFVTRKPFSTEKIKLNFKCST |
| effector | | | | | LLDGWDVNKETQNLGILLKKENAYYLGIMNKSDNKAFSKLPEYSGPDAYQKVMYKLLPGPNK |
| | | | | | MLPKVFFSKSRIEEFAPDESIIEIYKKGSFKKGADFSVQDCRKLIDFYKASIEKHPDWKEFGFRFSP |
| | | | | | TEEYNDISEFYREVSDQGYKITFKAVPEIFIDSLVSEGKLYLFKIYNKDFSPYSKGKPNLHTLYWK |
| | | | | | ALFDGRNLENVVYKLNGQAEVFYRKRSIPEDNRTIHPAGQPLSAKNPELQGKTSSFGYDIIKDRR |
| | | | | | YTVDKFQFHVPVTMNFTAAGTPRINEEVCDYIKNNPDINIIGIDRGERHLLYISMIDRDGNVVKDS |
| | | | | | SGRYIQYSLNTITGEYKNTEGEKVAFSTPYHELLGNRESERERARENWSVIENIKELKSGYLSQV |
| | | | | | VHHIAGLMADYNAVLVLEDLNSGFKNSRKKVEKQVYQNFEKAIIEKLNYLVFKDLSPDETGGLY |
| | | | | | HALQLTDRFASFRNLTKQSGFIFYVPAWNTSKIDPVTGFVDLIKPKYKNIPEAKEFFKKFESIKYN |
| | | | | | KEKDYFEFSFDYSRFTDKAEGTRTGWTVCTYGALRYAYEKSLNNGRGGYAKWNVTEKLKELF |
| | | | | | VRYGIDYKEGDLIGAIAEQNSSGFYATLIKCLQVTLAMRYSSAEDNKDFILSPVADRDGNFYCSEG |
| | | | | | RSDGLPQDADANGAYNIARKGLYVLNQIDRAENYKDWTTKISNKQWLSFVQSLN |
| MG29 | 6393 | MG29-70 effector | Protein | Unknown | MKKTLFDFTNIYSLSKTLRFELIPQGKTLENIEIKGLLKQDEDRAEKYKKVKIIIDEYHKDFIEKSL |
| effector | | | | | NGLILDGLHDYMSLYLMTIKEDKDKKAFDKEKEKLRKQIANAFKNNEKFKTLFAKELIKEDLM |
| | | | | | NFANEQDYEYIKAFKDFTTYFTGFHQNRENMYVVDEKATAIAYRLINENLPKFIDNLKIFEKIKN |
| | | | | | EAPGLINQLNKVLSEMEEIVQGKTLEEIFSLNYFNQTLTQTGIDLYNIVIGGRTPEENKIKIKGLNE |
| | | | | | YINTDFNQKQTDKKKKQPKFKQLYKQILSDRHSVSFMPESFENDNQLLESIEKFYTNELLHYSTE |
| | | | | | GKSINIFEAIKNAVGNLSSFNLSKIYLRSDTSLTDISQKVFGDWGMINKALQDYYEKIYPLKPKEK |
| | | | | | QEKYEERKNKWLKQDIDIQTLQTAIDHYENETVKEKNNGKIITDYFAKFGISDESKIDLLQNVYQ |
| | | | | | NYNIIKDLLNTPFPESEKLGSNKELVSLIKTFLDSIMNVIHFVKPLSLKDSDKEKDESFYSLFAGLY |
| | | | | | DQLNHTISIYNQVRNYLTQKPYSTEKIKLNFENSTLMDGWDLNKEADNTTIILRKDNLFYIGIMD |
| | | | | | KKNNHVFQHIPERTDNEPHYDKMIYKYFPDASKMIPKCSTQLKTVVSHFESNITDKIIEGKSFDSA |
| | | | | | LKITKRIFELNNFVYDDISKTMVLSEDNEKRPKMFQKKYLEISKDIDGFKDALKDWINFCIDFLN |
| | | | | | KYESTKHYSFNFKNSELYNSLDEFYGDIDTQTYKITYKNIPVSFIESLVNEGKLYLFQIYNKDFSPF |
| | | | | | SKGKPNLHTLYWKMLFDDENLNDVVYKLNGQAEVFYRKSSIKESNKIIHKANEVLINKNPDNK |
| | | | | | KTTSKFDYDIIKDKRYTLDKFQFHVPITMNFKADGILNINPKVNEFLKNNPAVNIIGIDRGERHLL |
| | | | | | YYTLINQKGEILEQDTLNVIANEKQKVDYHNLLDKKEGIRAEARKDWGTIETIKELKEGYLSQVI |
| | | | | | HKLTDLMVKNNAIIVMEDLNFGFMRGRQKVEKQVYQKFEKMLIDKLNYLVDKNKNNNEQGGV |
| | | | | | LHALQLANKFESFKSMGKQNGFIFYVPAWNTSKMDPVTGFVNLFDTRYENLEKAKVFFNKFNSI |
| | | | | | HYNQTKGYFEFEFDYNNYTAKAEGTKTNWTLCTYQNRIETFRNPEKNNQWDNREIELTNEFIKL |
| | | | | | FEQHGIDYKNNNELKSAIVMQTEKAFFERLLYLLKLTLQMRNSITGTETDYLISPVVNDKGEFYD |
| | | | | | SRNANNLLPQNADANGAYNIARKGLWCLQQINKTDDLKKIKLAISNKEWLQFVQNNN |
| MG29 | 6394 | MG29-71 effector | Protein | Unknown | MNAQNSIWDKFTNMYSLQKTLRFELKPIGRTLKLIKEKNLIEEDEEREKEFNQIKKIMDDYYKEF |
| effector | | | | | IELCLSKLNIPNQEIEEFKKTYDTLKKDHKNEKLKEQYAKNQTALRKLIYNSIKKTNNFNYLFGK |
| | | | | | EFINQTLPNWLEEKNRLDDKKLVLKFKKWVTYFEGFFDNRKNVFSEREIPTSIIYRIVHDNLPKF |
| | | | | | LDNISKFDELEKLSDFDYKSIEDEFKSELNGKSLREFLSLNNFENCLNQSGIERFNLIVGGKTTENN |
| | | | | | VKIKGLNEKINEYSQKQKDNKEQKKIRTLKLSPLFKQILSNRESESFILEKIKDKKELFEKIDNFY |
| | | | | | KSFNEFSNKLKNSVEKLKNCNHDNVYVKNDKQLTKISQEMFGNWDEINSGLRAYYGSKPKKTIK |
| | | | | | SLMKSKYFSINEIEEGLKTLETDNKQSIVEHFLNFTKKYNNTNINLFADISEKATEFYKINRNEKE |
| | | | | | KLTEKNIETIKEFLDSIMALYHFLKPLHLDLRKTEKEKGSEALETDSDFYNDFNEVFEELSQIVPL |
| | | | | | YNKVRNYVTQKPFSTGKFKLNFENPTLANGWDLNKEKDNYAVILRKINQKTKKYDYYLGIMSG |
| | | | | | SDKKIFEKNKNKNNNNDYYEKIIYKLLPDPKKMLPKVFFCEKNKAFFKPSEEILRIKNSSSHTKH |
| | | | | | GSPKTGFEKKEFDLNDCHKMIDFYKTSLEKHEEWREFNFKFKETNKYEDLSEFYADVANQGYK |
| | | | | | LSFTNIDKNYIDELINQEKLYLFQIWNKDFSEYSKGRPNIHTIYWRELFSEQNLNDIVYKLDGKAE |
| | | | | | LFYRDSSIEKKITHPKNTPIKNKNPIKNKETSKFPYDLIKDKRYSEEKFFFHCPITLNFKAKDQSKR |
| | | | | | IHKIVNNYIQKLGEKINVLGIDRGERNLAYYSLIDSEGKIIEQHSFNIISDKLKRKFNYQERLDEIEG |
| | | | | | NRDKARKNWKKIENIKEMKTGYLSQVIYNIAKLTIEHNAIIVLEDLNFGFKRGRFKIEKQIYQKF |
| | | | | | EKMLIDKMNYLIFKDRKENQTGGCFKAYQLTNKFESFKKLGKQSGIIYYVDAYKTSKVCPKTGF |
| | | | | | VNLLYPRFKNIQKSKEYLKKFKYIKFSPEDNLFELNFNYSNFFSENKNKLIRDNWSIWSNGIKLVQ |
| | | | | | KRNKDKNNSWETKEINVTKELQELFHQNNIDYSSGENLIKQIINIENKSFYETLLNLIRLILKLRNS |
| | | | | | YSDYEIKNFKKNLGDQFNESNYDYILSCVKDKDGNFFDSRNAKDDEVKDADANGAYHIALKGLM |
| | | | | | LINKIKTADTSKKIDLRIDRNDYLNYIIKKAN |
| MG29 | 6395 | MG29-72 effector | Protein | Unknown | DETSANDVEVIKDFLDSIMNIYHFVKPLHIDFKKSEGDKGTDALDLNADFYNGFDEIFGKLGEIL |
| effector | | | | | LYNRVRNYVTQKPFCTKKFKLNFKSATLASGWDLNKEHDKNYSFIFKEGKSFFLGVINVEEDKN |
| | | | | | VLREDKHPEIFIKNSSFMKMIYKDLGNINKQISRLGFSEKAKKGVETVGWNPEIKSIKKEFEEFQ |
| | | | | | KSKEKDKSGRSSNFDKDKLFKLIEYYKSVLKNHSEKYEETYNLSYKPTKDYKNLGEFFDDISSQT |
| | | | | | HKMEFVGIDKNYVNKLIDEGKLYLFQIWNKDFSDFSIRNEMDPNKKSKPNLHTIYWREIFSEENI |
| | | | | | KDPVHKLNGGAELFFRKASSKRDITHPKNQEIKNKNPINGKEKSTFIYDLIKNERYTEDKFFFYCP |
| | | | | | VTLNFKVGEKNKVLNRLVNKYIHNTNEEINILGIDRGERNLAYYVLIDSRGNILEQNSFNIISDDLH |
| | | | | | RKLNYQEKLDFIEGERDKARKTWKNIVNIKEMKTGYLSHAIHKIAKLAIESNAIIVLEDLNFGFQ |
| | | | | | RGRFKIEKQIYQKFEKMLIDKLNYLVFKERNNNQSGGSFKAYQLTNKFDSFKKLGKQSGIIFYVD |
| | | | | | AYKTSNICPKTGFTNLLFPKFENVDNSRNFFKKFKFIRFKNDEDLFEFNFNLFDFSSENNKSRLLR |
| | | | | | DNWSVWSNGTKLIKTKDKENNYNWKTKEINVTKELKELFDYNEIKYTSAENIVDQIIRVEDRSFY |
| | | | | | EKLTELFKRVLQLRNSYTDYEIKEFKKKLGDGFRMSNYDFILSCVKDRKGHFFDSRNAQDHEVK |
| | | | | | DADANGAYHIALKGLMLIEKIKKSDTNEKLDLKIDRFDFINYAVTRAI |
| MG29 | 6396 | MG29-73 effector | Protein | Unknown | MKNFTNIYPQSKTLKFELRPQGATLDNIHKSGLIDQDETLKADYQAVKKMIDEYHKVVIDESLT |
| effector | | | | | NFKLTGLPAYEELYYKDRTEAEDKTFEKIQSNLRKQVHKAFGENKRYKSIFKKELIQKDLPDFV |
| | | | | | NKEEEREQISRFYHFTTYFTGFHENRKNIYTAEAKATSVCNRLIHENLPKFLDNRKTYLNYISNFG |
| | | | | | DLDMSQAEEDLQEVLGGIKVGELFSLDFFNHTLTQQDIDIYNIALGGRSVEGGKKIQGINECINLY |
| | | | | | RQNNQLKARQLPNIKPLYKQILSESESGSFLLDKFEKDEDLFDGLRNFYQGLNSFNYKGEQDKST |
| | | | | | FIELMNLFGRFSESDMTRVYLRNDASLSRLSKKLFGDWSLIVSALRYYYDAEVNPLMGKKATNK |
| | | | | | YIKEKENWLNKSRDFSIDIINKSLLRYGTINETVNSQFTDDIIFEHFSSFMIEEKNLLNTVAENFML |
| | | | | | VSEVLSRGSLDKDQNKKKKEIKTIKTFLDNVLDLLHFIKPLSVQHVGAEKDEGFYSDFDVLYDQL |
| | | | | | SQVIPLYSKTRYYLTKKPYSVAKFKMNFKNNTLLDGWDVNKETANKGVLLQKEGLFYLAIMNK |
| | | | | | DHSKSFYNIMDTGDTTGYQKMDYKLLPGPNKMLPKVFFSVKNLGFFNPSEKVLRIRNTSSHSKN |
| | | | | | GNPQEGFDKADFSLNDCHSLIDFFKASLDKHADWKKFAFDFSPTQSYNDISEFYREVENQGYKIT |
| | | | | | YTNISDEYIHELVNEGKIYLFQIYNKDFSPFSKGKPNLHTLYWRALFDEKNLEDVVYKLNGQAEV |
| | | | | | FYRKKSIEYSEEKWIQGHHYDQLKDRFAYPIIKDKRFAFDKFQFHVPITMNFKASGSPVINMKVR |
| | | | | | EYLKTNPDVKIIGLDRGERHLLYLTLIDQNGNIEEQYSLNEIVNSYNGKVHKKDYQQLLHEKEG |
| | | | | | DRKKARENWETIETIKELKEGYLSHVVHKIVNMMVEHNAIVVMEDLNFGFKRGRFHVEKQIYQ |
| | | | | | KFEKMLIDKLNYLVLKDTQDPKAPTGLLNALQLSNKFESFQKLGKQSGFVFYLPAYLTSKIDPAT |
| | | | | | GFVNQLRIKYDSVVKSQAYYRQFDRIVYNTTTDWFEFGFRYVNFGNTTPSVRQEPWTICTTHHP |
| | | | | | RYAWNKNSNMGKGGTEEYNITEELKKLFDHHNIAYEEGTDLIESIASNTGTDFFKRLNKLLNVT |
| | | | | | ASLRHNNGKKGKEERDFILSPVANSEGAFFNSLEADETQPENADANGAYHIALKGLWALQSIRK |
| | | | | | TDTDRMAKLNLVVSNEEWLNFAQAKQYRSQSSS |
| MG29 | 6397 | MG29-74 effector | Protein | Unknown | MGIGKIDNMLKEELKTSISKEGGNNNNSKSLTFYNVSNFEKSKESIWDEFTNLYSLQKTLRFELK |
| effector | | | | | PLGKTKEFIEKKGLVEEDEEREKEFNQVKKIMDDYYREFIEICLSEIKIETEDIEALERVYNGLKK |
| | | | | | DTKNQTLREEYTKYQKDLRAKIYDKIKKIKNFGVLFGKDFINKILPDWLDCKNRQQDKDLVLKF |
| | | | | | KRWVTYFNGFFKNRKNIFSEKEIPTSIIFRIVHDNMPKFLDNVSRFKEARKLIDFDYKKIEENFKSE |
| | | | | | LNGQSLEEFFSLDNISNCLNQAGIELHNLLIGGKSLEHNLKIKGVNEYIEELSKTLQEKTEQKKIR |
| | | | | | KLKLSPLFKQILSDRESASFVLEQFKDKNEVFQKIDEFYVHFNNISDKIQSVIAKLKECDVDSVYL |
| | | | | | KNDNNLTKISQNIFGDWNKIHEGLKEYFILESKSKDLTEKQCIKEVEKKMKSKYFSISEIEGGISLI |
| | | | | | NVEKKQSLIDYFLNFSGVIDNANMNLFEDLKEKYSAFKQIDRNKDGDLKDETSANDVEVIKDFLD |
| | | | | | SIMNIYHFVKPLHIDFKKSEGDKGTDALDLNADFYNGFDEIFGKLGEIILLYNRVRNYVTQKPFCT |
| | | | | | KKFKLNFKSATLASGWDLNKEHDKNYSFIFKEGKSFFLGVINVEEDKNVLREDKHPEIFIKNSSF |
| | | | | | MKMIYKDLGNINKQISRLGFSEKAKKGVETVGWNPEIKSIKKEFEEFQKSKEKDKSGRSSNFDK |
| | | | | | DKLFKLIEYYKSVLKNHSEKYEETYNLSYKPTKDYKNLGEFFDDISSQTHKMKFVGIDKNYVNK |
| | | | | | LIDEGKLYLFQIWNKDFSDFSIRNEMDPNKKSKPNLHTIYWREIFSEENIKDPVHKLNGGAELFFR |
| | | | | | KASSKRDITHPKNQEIKNKNPINGKEKSTFIYDLIKNERYTEDKFFFYCPVTLNFKVGEKNKVLN |
| | | | | | RLVNKYIHNTNEEINILGIDRGERNLAYYVLIDSRGNILEQNSFNIISDDLHRKLNYQEKLDFIEGE |
| | | | | | RDKARKTWKNIVNIKEMKTGYLSHAIHKIAKLAIESNAIIVLEDLNFGFQRGRFKIEKQIYQKFE |
| | | | | | KMLIDKLNYLVFKERNNNQSGGSFKAYQLTNKFDSFKKLGKQSGIIFYVDAYKTSNICPKTGFTN |
| | | | | | LLFPKFENVDNSRNFFKKFKFIRFKNDEDLFEFNFNLFDFSSENNKSRLLRDNWSVWSNGTKLIK |
| | | | | | TKDKENNYNWKTKEINVTKELKELFDYNEIKYTSAENIVDQIIRVEDRSFYEKLTELFKRVLQLR |
| | | | | | NSYTDYEIKEFKKKLGDGFRMSNYDFILSCVKDRKGHFFDSRNAQDHEVKDADANGAYHIALKG |
| | | | | | LMLIEKIKKSDTNEKLDLKIDRFDFINYATKRYI |
| MG29 | 6398 | MG29-75 effector | Protein | Unknown | MNNSIWNEMTNKYSLSKTLRFELKPIGKTEEFIEKNGLIIEDEQRNRDFLYGKELLNEYYSYLIEK |
| effector | | | | | RLGEIKIDLGILKDYYEKYKNFSKLKQKGKVVDSKELKKSEIELIDIQNHLRKALHKVFFFDKNL |
| | | | | | SDKRDEKDIIAAVKYHVENGLVKEKSDRLNNFITNRCTTYFTGFFNNRDNVFTSDEIPASVFYRTI |
| | | | | | NENLPFFIKNIEKFEKLKNLIPENEFIALEKNLKNELGEFSVKEVFSIKYENNCLNQKGIDKYDQI |
| | | | | | MGGIKKETIQIKGFNGLINEFSQKGDKNIRKLRMTVLYKQILSKSDSKSFKIESIKNGKELKDIIN |
| | | | | | GFYDSICSLNKDLDESIIISLENLLKADKDLDLSGIYINSKKLKDISNKVFGDWYLIESALKQQYKS |
| | | | | | QIKSKGKKEKTESKKDEETEEWFKKLKQFSISEINSSLSNIENDLVTKENNTIWTYFSTFEDSNGK |
| | | | | | NLVNEIKNSFKELKTIQFGEDKELLNDDNEENVRKIKKALDSVQELFWFISPLMYDKPKEEVFDL |
| | | | | | DLDAGFYEKFNVIYEGLRQIIPLYNKTRNFIAQKPFNVSKFKLNFENSTLLNGWDRNKEADNWSI |
| | | | | | LFRKDDNYYLGIIASGKGNNKIFEKIPEYKEGDYYEKMNYILWSNPSRMLPKVFFANTNFDLYNP |
| | | | | | SEEILNIRKNSSFTKNGEAQKGFDKNEFNLNDCHKMIDFYKNCFNKHPRWSNFGLVFKNTKDYL |
| | | | | | DISEFYKDVADQIYQLSFQKVSSKFVDELVNEGKLYLFKIWSKDFSEYSTGKPNLHTMYWRELFS |
| | | | | | DNNLNNVVYKLNGQAEIFFRKKSLPIKITHPKNINIKNKDPIKDKENSVFKYDLIKDKRYTENKYL |
| | | | | | FHCPITMNFKAKGNSFINNEINEFILKNHNKINILGIDRGERNLLYCSLLDPSGKILFQKSFNVMPD |
| | | | | | KFGRDVKYYDKLDAKEKERDKARKEWKNIKNIKELKEGYLSQIIHEIAKLVIRYNAIVILEDLNF |
| | | | | | GFKRGRFKFEKQVYQKFELKLIEKLNYLAFKDKKPTETGGLLHAYQLTNEFESFKKLGKQSGIL |
| | | | | | YYVGASYTSKIDPKTGFVNLLHPNYENLNKSKEFFEKFDSIKFNKNEDMFEFDFKYSNFNKESKL |
| | | | | | EKDQWEIFTNKERIIHERTSNNNYSPKNINLTEEIKKLFDNERIPFADGINLKEYIISSDSKNLHKG |
| | | | | | LINLLKYTLQLRNSNSETGEDYILSCVKYDKKNFFDSRNAKENEPKDADANGAYNIGLKGLMLIE |
| | | | | | KIITQNNNKKQINEKQKYDFKMSNEEYFNWVIRRNLTK |
| MG29 | 6399 | MG29-76 effector | Protein | Unknown | MDSNITSFSFKYSLSKTLRFELKPVGKTLENIQNKGLIAKDEERSKSYARMKKTIDAFHKYFIELA |
| effector | | | | | MKEVELTKLNEYVEHYYESAERKKEDSFIKSFDKVKADLRKEIVKGFNSDKVKEIFSKIDKKELI |
| | | | | | TELLEDWIQNQPNKEEIYFDEGFKTFTTYFGGFHENRKNMYSDKEQSTAIAYRLIHENLPKFLDN |
| | | | | | IKNFEDAVQKLGEEKIAEIEKTLEPILQGKTLGEVFILEYFNHTLTQTGIELYNTILGGYTQNEGR |
| | | | | | IKIQGLNEYFNLYNQTQQDKKNKAAKLKPLYKQILSDRGTTSFLIEKFENSQEVLDAINEFYLFNL |
| | | | | | IEYKPEDKDETENVLSKLKELLAELSQYDLSKIYLKNDRAITDISQTIFGDWNIIKVALEQHWLNA |
| | | | | | NTDKNKALTKKQEEAKSKFLSKSQFSIAEIEAALFAYKNEADVLKDLKESDHPIADYFKTHFKAK |
| | | | | | KEKETDKDFDLIANIDAKHSCIKGVLNSDYPEDKKLYQQQKDIDNIKIFLESIMELLHFVKPLALT |
| | | | | | KGNSLEKEDKFYGQFETWFEQLSLLTPLYDKVRNYATQKAYSIEKFKLNFESGYFFSGWGIDYD |
| | | | | | SKSCLVVIKDENYYLLIVDKKLQKSEIEFLKENVNKNPAQRVIYDFQKPDNKNVPRLFIRSKGDRF |
| | | | | | APAVHRYNLPIDSIIDIYDNGYFKTEYRAQNLSHFKECLSKLIDYFKLGFERHESYKHYQFNWKN |
| | | | | | STQYNDIAEFYKDVINSCYQLKLEEINFDNLLELINLQKGYLFQIYNKDFSEYSKGKPNMHTMYW |
| | | | | | RALFDEENLKDVVYKLNGEAELFYRKKSIDDDKKIIHKANEKLANKNPLNPKRESKFEYELIKDK |
| | | | | | RFTVDKFSFHVPITMNFKADGNDYINQDVLKFLKNNPDVNIIGLDRGERHLIYLTLINQKGEILDG |
| | | | | | LQFSLNEILNTYKNSKGENVELKTSYHTLLDAKEKERDEARKTWKSIENIKELKEGYISQVVHIIA |
| | | | | | KMMVEFNAIVVMEDLNFGFKRGRFKVEKQVYQKLEKMLIDKLNYLVFKDVEESKSGGLYNAL |
| | | | | | QLTNKFTSFKEMGKQSGFLFYVPAWNTSKIDPTTGFVNLFDTRYESIEKAQQFFKKFKSIQYNSQ |
| | | | | | KQYFEFAFDYSEFTNRAEGSKINWKVCTQGDRIITFRNPESNNQWINKKVNLSQEFEDFFGKHN |
| | | | | | VIYGNGENFQPQIEAQTTKEFFEKLLQLFKLTVQMRNSKSDEDYLISPVINKTGEFYDSREANDT |
| | | | | | LPKDADANGAYHIAKKGLWVLEQINETEDYKKLKLAISNKEWLQFVQKNN |
| MG29 | 6400 | MG29-77 effector | Protein | Unknown | MQKNVFDTFTCLYSLSKTLRFELRPIDENGRWITDQRFENFTSALQGIIGNDNQRAKYYKQAKPL |
| effector | | | | | IDEYHRYYIDEKLESPVDPKTDEAVLDEDNIVKAFECYRELRNAAGNRRQDTEAFQAAQKKWSK |
| | | | | | FQDVLRKSLVKCFGDKKDHLFSKTLIKSTLPEWLEKQGRWEENRACVEAFEKFTGYFIGYNQN |
| | | | | | RENMYVDEPQKTAIAFRLMNENLSKFFNNCLAYEKIRKNHSDLVFSADNEIPEKSHVADVLTILE |
| | | | | | PGFFLKLFTQSGIDAYNTLIGGINKQINLYRQKHDIKPRNLPNLTPLYKQILSDRESHSFILEEFEN |
| | | | | | DQNLLDTLRDFVNPARALIEKLKTTLETLPDSDLDRVYIRGSELRTISQWLFNDYSFIGSAQTLHA |
| | | | | | ENIFPSSPKGKVTKKLEKEREKYPKQDVFSIAELDDALKSYIELRKQESPDEEFLSGVDTNFPVRN |
| | | | | | YFLTEAKKWKNHQGLQDAIQKFETVCSSNKISADRRAPGSDNETDGGGEGYRQVQSVRNMLDE |
| | | | | | FMTFLQLTRPLHLVRKGKPIDIPDMDMSFYADFSAAYENYQDRLLPLYNKTRNYLTKKPYSPDK |
| | | | | | IKLNFDKPNFLEGWDLNKEKANLGVLLRRDDNYFLAVMRPEHNNVFRNYPTPEGDEEIYGKINY |
| | | | | | KQISGMNKMLPKVFFSKKWRKEHDVPGEIDTLYKNEEHIQNENFNQESLHKLIDFFKARINEYE |
| | | | | | KDGFPWRNFDFHFPDTKEYQNIADFYREVEEQAYKIWFIKVPVSHIENLVREEKIFLFQIYNKDFS |
| | | | | | QHSKGRPNLHTLYWRALFEKKNLNNVVIKLNGGAEVFYRPHSIKENERIIHKAHQSLNNKNKNN |
| | | | | | PRKTSCFAYEIVKDRRYTQDKFLFHVPLTLNFKAPKAKSLNDCVNNAIRNEPDVHIIGIDRGERH |
| | | | | | LLYLTVIDQKGNIVKQCSLNEIETDKNYNVQYREKLDQKEKERDKARKSWSSIENIKDLKSGYLS |
| | | | | | QIVHSLAKLIIEYNAIVCLEDLNFGFKRGRFMIEKQVYQKFEKALIDKLNYLVFKDAKPGQAGHY |
| | | | | | LKAYQLTERFRSFEKLGKQSGILYYVPAAYTSKADPATGFVNFLYPKYESLEKSKQFFSKMELIR |
| | | | | | YNTGDEYFEFTFKYSNFPIRQDLKNYPDEWTICTHGDARYWNKRENNRWISEEINVTQQLAALL |
| | | | | | DSKNIDYRDGRDIKQDIAAAKDTKFYKKLYRLLQVTLSLRYSKSGTDIDFILSPTRDKSGTFFDSR |
| | | | | | QAPSTMPENADANGAYHIALKGLWHLQQLRKQEEGQNLKLEKITNSEWFDFLIQQVKKS |
| MG29 | 6401 | MG29-78 effector | Protein | Unknown | FTKTTPEVHLAHHVIYAFPKPENKNVPRILIRSKGTSFAPAVQKYDLPVESIIHIYDEGLYKTDFK |
| effector | | | | | KENPAVFKKSLVQLIDYFKLGFSRHDSYKHYDFEWKESEEYENIADFYQDVINSCYELRDEAINF |
| | | | | | DHLHQLIDQGKLYLFQTYNKDFSKHSKGKPNLHTMYWKALFEKQNLKDVVYKLNGEAEMFYR |
| | | | | | KKSIQDKNRVVHKKNIKVARKFYKDDKKTERVPDETVLRLNKFYKGQIQESGLKKEDLKFKDN |
| | | | | | YSLFHEQGKDIDLIKDKRYTVDKFQFHVPITLNFKAKGNDYINNEVLDYLKDNPDVNIIGLDRGE |
| | | | | | RHLIYLTLIDQEGNIIEQETLNSIVNKKHAITTNYHQLLDDKERERDKARKNWGTVETIKELKEG |
| | | | | | YISQVVHKIAKMMVEHNAIVVMEDLNMGFKRGRFKVEKQVYQKLEKMLIDKLNYLVLKDKKP |
| | | | | | DEPAGIYNALQLTNKFESFQKIGKQSGFLFYVPAWNTSKIDPTTGFVNLFHVKYESVNKAKTFFS |
| | | | | | HFKSIRYNKNQGYFEFDFDYNDFTTRAEGTRSEWTVCTYGERIKTFRNPDKVNQWDNQEINLTE |
| | | | | | AFEDFFGKHEIVYGDGTDFKDQISVKDNKDFFAELIHLFRLTLQMRNSITNSEVDYLISPVKNSSG |
| | | | | | TFYDSRKADASLPKDADANGAYHIAKKGLQWVNQIQEFDGDDWKKLKLNKTNKGWLKFVQCN |
| | | | | | T |
| MG29 | 6402 | MG29-79 effector | Protein | Unknown | TKNLIQEKENVAEIKTLLDLIMELLHLCKLFVIKADIAEKEHNFYSELEYCLELLNPIIALYNQTR |
| effector | | | | | NYLTQKPYSLEKFKLNFENATLLDGWDRNKETSNTSILLRKNGNYYLGIMNKRHNRVFEALPQI |
| | | | | | LKTNNSYEKIVYKLLPGANKMLPKVFLSTKGKATFQPSQEILDNYKSETHKKGDNFNLDHCHKL |
| | | | | | IDYFKDSINKHSDWKNFNFNFSETSSYEDISGFYREVEAQGYKIIFDLISEQYIDELINEGKLYLFQI |
| | | | | | YNKDFSPHSKGTPNMHTMYWKALFSEENLANVVYKLNGQAEIFYRKKSITADKTIKHLANQPIN |
| | | | | | NKNINNAKKQSTFAYDLIKDKRFTMDKFQFHVPITLNFKAQGRDDINLEVKEYLKSNPHTHVIGI |
| | | | | | DRGERHLLYVSVVNPQGEIIKQFSLNEIINEYKGNTYKVDYHNLLNNKEGDRKKARENWGVVEN |
| | | | | | IKELKEGYLSQVIHKICQLVIEYQAIIVMEDLNSGFKNSRIKVEKQVYQKFEKMLIDKLQYLAFK |
| | | | | | NPQENQPSIYGALQLASKFESFQKLGTQSGFIFYVPAWNTSKIDPATGFVDLLKPKYETVAKAQE |
| | | | | | FIHKFDDIKYNQSKDWFEFNFNYSKFTEKADGTRLNWTICTTNINRYSWNRKLNNGKGGQQLFE |
| | | | | | ITKCLKALFDNYKINYQSGENLVVQIATQSEKDFYVCLLKYLSITLNLRHNNGKSGIDEEDYIASPI |
| | | | | | ADNAGAFFDSRNEVIKGKNEIGDWLSKLPVDADANGAYIIIAKKGLWVLIIQLEKADNLRKVKLA |
| | | | | | ITNKEWLEFTQN |
| MG29 | 6403 | MG29-80 effector | Protein | Unknown | MKLNKFTHQYPISKTLRFELKPVGETADYIEDFKSQYLKDIVIQDQQRAEDYESIKVIIDEYHRHY |
| effector | | | | | IEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSHEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNDLLKSL |
| | | | | | GKFIKEMVKEDGLFKKLEDSIKLITDADLHRTFIKNGVEITKISQSIFGNYSILKSAIYHHAESVLY |
| | | | | | PDPISGKISEALKEKRIKYVNKQVVFSIAELETILSNYASQMTDDNPDKEIIAQCENSEHPIRTYFL |
| | | | | | NAIENVKNDKDIEFGKAIENVLPLISLENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEEGKLFLFQIY |
| | | | | | NKDFSPYSKGKPNLHTLYWKALFDTENLKDVVAKLNGQAEIFYRKHSIKKDERTIHRANKSLQN |
| | | | | | KNENNPKKTSLFEYDIIKDRRYTVDKFQFHVPITLNFKMEKMTQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDSRNATKDHPMDADGNGAYHIVLKGL |
| MG29 | 6404 | MG29-81 effector | Protein | Unknown | NYPPTITYSDEVKKILKRLPDRNAGDDSVTICSTDDAMEPKKSIWDDCTNLYSLQKTLRFELKPIG |
| effector | | | | | RTKDFINQKRLIEDDEELAKKFNEAKKIMDDYYKFFIEDRLKKVRIEECDLKEFKKIYQDLKQSR |
| | | | | | GKDKKIRAEFSELQEKIRNDIYKKFFTETLEKFLFEKNFLTKILSEWLESNNRKEDSNTIEEFQRG |
| | | | | | TTYFTGFFDNRKNVFSKDDIHTSFIYRIVHDNLPKYIDNLERYDKLKQYSDFNYEQIGKDFSSELK |
| | | | | | SQTLSEFFTLENFNECLNQSGIERFNLIIGGKSLNNNKKIQGLNEAINLYSQKTNEENKRAVRKLF |
| | | | | | MRPLFKQILSDRTSASFILSVIEDDAAVIFSINELYSNISKDFDLLQNIFKGLFEFNTGQIYINKKLLN |
| | | | | | HVSKQIYDDWGILEISLKCFAKEKLNLTTEKKIEEWFKKSAYFSLEEIKQGIECLKSENAAWEKF |
| | | | | | VEYHRAITRDNKQLIDAIKEEYTAVKGLNLNKGERTLLTEQKEDDVTKIKQFLDSVMNIYHFLK |
| | | | | | PFIMVLDADSEKESFTVEIDTEFYKDENDIYDRLCEVVPLYNKVRNYVTQKPFRTNKFKLNFNES |
| | | | | | TLLGGWDKNKEHNKNYSFIFRENDSFFLGITNTDEDKNILREDKHPEIFVENSLFKKMICKDLGD |
| | | | | | IKMQLPRIGFSDKAKNGVEDVGWNQEIQEIKNEFDSFQEQKKNDKDLWTEKFNRDKLFRLIDYY |
| | | | | | KGVLKNHSERYEKTCNITYKSTSNYRNLGDFFNHVSFQTYNVKFVGIDKSYIESLVSEGKLYLFQI |
| MG29 | 6405 | MG29-82 effector | Protein | Unknown | MKNLSEFTNLYSLQKTLRFELIPVGKTQKHIEYNGIIDCDTKRAESYKKMKKTIDKYHKDFIESS |
| effector | | | | | LRKASLSGLDEFYVLYTSNTEEKKTDAYKKRFSAAKQSLRKEIVKRFKDGPKKDIFKNLDKKKLI |
| | | | | | QDELEKWLQEKDNELYFNSDFKSFTTYFTGYNTNRMNMYSDEEKSTAIAYRLINENLPKFIDNM |
| | | | | | LIFEEIKNTTIYNKFPQIYEEIKEYLYVTRIEEIFELEYFNNTLTQTMIENYNAILGGVKIGNRKIKG |
| | | | | | LNEYINEYNQKSKDKRIPNLKMLYKQILSERETFSYLPEEFESTQEMLDAIAKFYENQLCSYSDEY |
| | | | | | SGEEINIVDSLKSIVSSLREHKLNKVYIRNDSSVDTISNRLFGDFSVIKASLEYYYENVVAPGFQESY |
| | | | | | KKASDKKRKQLEKQKKDYISGPAYFPIGLIQDALGCYVEQLDKNEYANIHAKYSDSCVADYFSA |
| | | | | | FEAKDGVNLINELNQKYKKAQDLFNISFPDGYNLLDYEKGILKAFLDSILSLLHFVKPLYLTPDSE |
| | | | | | LSKDELFYSAFSPLFEQLQKITEIYDKTRNYLTKKPYSTEKVKLNFSNPTLLEGWDVNKEPDYGA |
| | | | | | VLFERDGLYYLGILNDRNLFKNIESRFDLHSSEKGSYRKIVYKQIPDAAKYISSKQILPQNPPQKII |
| | | | | | NILEKKKTNAQSLKREEIHSFIDYCKNDFLKNYSCLKDANGKDYFDFNLKPADEYETLKHFFDDV |
| | | | | | KKQAYSISFLGITEKFINEAVEEGKLYLFKIWNKDFSSFSKGKPNLHTMYWRALFADENLKNVSY |
| | | | | | KLNGNAELFHRKASISKDNMVIHKANQPIACKNPNLKQKTSVFEYDIIKDRRYTCDKYQFHVPIT |
| | | | | | LNFKANGQTNINERVLKYLKNNQDINVIGIDRGERHLLYISLLNREGNVIMDEDGNPLQYSLNDI |
| | | | | | VGVYRDNSGALVSIKTPYRELLDKREIKRKDARKNWETIERIKDLKEGYMSQVIHHIAKLMVKY |
| | | | | | NAIIVMEDLNSGFKNSRVKIEKQVYQKFEKLLINKLNYMIFKDLPEKEPGGLYKAFQMINKFESF |
| | | | | | KRMTRQNGFVFYVPPWNTSRIDPVTGFVDLLKPKYTNIPDARSFFSKFDSISFNSQSDYFEFEFDY |
| | | | | | SQFTEKANGTRTRWTVCTYGNERYTYNRSLNGGLGGYEKWNVTEKLKSLFDNVSIYYNSEENLI |
| | | | | | PQIISQENSAFFLELIKNLKITLALRYSDGNDRDFILSPVSNEGRFFYSEECEKTLPQDGDANGAFH |
| | | | | | IARKGLCLLKKIDLMEDLKKPDLKISNKEWLSFVQTRLV |
| MG29 | 6406 | MG29-83 effector | Protein | Unknown | MKLNKFTHQYPISKTLRFELKPVGETADYIEDFKSQYLKDIVIQDQQRAEDYESIKVIIDEYHRHY |
| effector | | | | | IEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSHEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNDLLKSL |
| | | | | | GKFIKEMVKEDGLFKKLEDSIKLITDADLHRTFIKNGVEITKISQSIFGNYSILKSAIYHHAESVLY |
| | | | | | PDPISGKISEALKEKRIKYVNKQVVFSIAELETILSNYASQMTDDNPDKEIIAQCENSEHPIRTYFL |
| | | | | | NAIENVKNDKDIEFGKAIENVLPLISLENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEEGKLFLFQIY |
| | | | | | NKDFSPYSKGKPNLHTLYWKALFDTENLKDVVAKLNGQAEIFYRKHSIKKDERTIHRANKSLQN |
| | | | | | KNENNPKKTSLFEYDIIKDRRYTVDKFQFHVPITLNFKMEKMTQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDSRNATKDQPMDADGNGAYHIALKGLWNLEQIRNWDGESRLNLAMKNVDWFSFAY |
| | | | | | QKPFKK |
| MG29 | 6407 | MG29-84 effector | Protein | Unknown | LSSFTRKYPLSKTLRFELKPVDDTKKYLKEFVQYDQKRAEDYKKLKTIIDEYHKDYIEKSLSKND |
| effector | | | | | ILSLDDLNSLKEHIEKSGSLQLLEEKQKKEKSIKELQNTLRGQIVKDFQIDTKQEKDRLFGAKLIK |
| | | | | | DVLPEWLDSGSLENIEYKKEIVKEFQKFNTYLTGFHENRKNIYSDKEQSTAVSHRIINENLPKFLS |
| | | | | | NLNTYEKIKTNFPELKAQFESLKKDLKAEFEYFKIQNIKDLFKIQFLNKCFTQTGIDNYNSIVGVR |
| | | | | | VLESGQKIQGINEKINLFRQAQSSKGNQETRKISNTSLPVMQTLYKQILSDRESHSFYFEEFKSRN |
| | | | | | EVLESINCYWESIFNKERQSEYIAKRQTRHKEGDQSVLQRMENLFTNLDKYELDKIYFKSNDLSF |
| | | | | | VSKKLFEDYSVIGSALNFHAENKFSKKKEKEQYVKKDFFSFDEIQTSLSDYLKENEEIKFSSRFPG |
| | | | | | LKEIEKDLSVDPKKEQSNVLVVYFNLEFSKKKYNNKSLLDFIEELYKHVKKEIPPSLQTQNEFNK |
| | | | | | KEVQIIQNYLKPFIPNCPENSDSYNGKEKNETLKQGQYNTERGSVGLMDLLHLIKPVYLEKDKK |
| | | | | | KIMDLEKDASFYNEFEELYNKLSQIIPLYNKVRNYITKNKRCTEKIKINFEKSTLLNGWDINSETD |
| | | | | | YLSVILRKKDKVIGWKYYLGVMNTHRFEKANRMFDYHIKSDKSDKKQQEELRQKILHKEDNEN |
| | | | | | FYEKMNYKLLPKPSKMLPKVFFSKRNLSFFSPSPEILEIKKSKRSYSKNNGDTFSLNDCHKLIDFY |
| | | | | | KKSIQKHKEWGAIFNFNFSHTRNYRDISEFFAEVASQGYRLSFDKIKSSYIEGKVKTGEFLLFEIY |
| | | | | | NKDFSTHSKGRPNLHTSYFKLLFAEENLKDIVYKLNGHAEIFYRKASKQKKISYKAQVPIKNRNP |
| | | | | | DNQKKTSQ |
| MG29 | 6408 | MG29-85 effector | Protein | Unknown | LRKEIVKGFSIGEAKEIFNKIDKKELITDLLEQWVKKQSDDFYFDENFKNFTTYFGGFHENRKNM |
| effector | | | | | YSDKAQSTAIAYRLIHENLPKFLENIKIFERIKAVPKVYEKCQTLYKEIEEYLNITQIDEAFELDYY |
| | | | | | NEVLTQTQIDVYNLIIGGRTAEEGKKKIQGLNEYINLYNQKQEKNKRLPKLKLLYKQILSDRISTS |
| | | | | | FMAESFSEDQEVIDAIEEYYKFHLLAFQAEDKDDTENILEKVKELLSNIKEYDLSKIYLRNDTKIT |
| | | | | | AISQKIFGNYGVFNTALEYYYATAVKPDFQKEYEKANQKKRDTLDKAQTQFVKQPYVSIELLQT |
| | | | | | ATDAYIATIDKGEEIYKRYSPTCIADYFKNNFKAEKKEKNDKEYGFIDNIKAKYSCIQGILGTPYPK |
| | | | | | DKKLIQQKNDIPNIKAFLDSLMELLHFTQPLSIVDEREVTKILTSDRLKIDKEWAEEANKSFFEKD |
| | | | | | KKFYEQFDLYFKELQKLIPLYNKVRNYATKKPYSTEKFKLNFENKGQFLGGWVDSHTENSDNA |
| | | | | | TQAGGYLFRKKNQIGEYDYFLGVSSDSKLFRSHLRNEIVDEDKSEFERLDYYQLKSASVYGNSYI |
| | | | | | GNQSYDKDKENLFDSIIEFANRNNPAAKEDFNKYISSQKGDNKPTPNGLLKILQEKHSKALEELM |
| | | | | | IDGDFIRINTIVTDNLKNTILSLNRIPKSQEYKNTIFTLFTEPIQVIEELSKEKSFSYFPVSAKELEDS |
| | | | | | LNRELKPLLLFKISNQDLSYADSFSQGKRKSRGRENMHTLYFRQLMSGSQNILDIGTGEVFFRKS |
| | | | | | SIENPTIHKANEAVTNKNPLAKKKDSAFEYDIIKDRRFTVDKFQFHLSIIMNYQKPQKASDENFEV |
| | | | | | LEFLQNNPNVNIIGLDRGERHLIYLTLINQKGEILLQESLNNISSENYPITTPYHDLLATKEKERDE |
| | | | | | ARKSWGTIENIKELKEGYISQVVHKIAKLMVEHNAIVVMEDLNFGFKRGRFKVEKQVYQKLEK |
| | | | | | MLIDKLNYLVFKDKDPNEIGGLYKALQLANKFESFQKMGKQSG |
| MG29 | 6409 | MG29-86 effector | Protein | Unknown | MKLNKFTHQYPISKTLRFELKPVGETADYIEDFKSQYLKDIVIQDQQRAEDYESIKVIIDEYHRHY |
| effector | | | | | IEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSHEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNDLLKSL |
| | | | | | GKFIKEMVKEDGLFKKLEDSIKLITDADLIIRTFIKNGVEITKISQSIFGNYSILKSAIYIIIIAESVLY |
| | | | | | PDPISGKISEALKEKRIKYVNKQVVFSIAELETILSNYASQMTDDNPDKEIIAQCENSEHPIRTYFL |
| | | | | | NAIENVKNDKDIEFGNAIENVLPRISVENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEDGKLFLFQIY |
| | | | | | NKDFSPYSKGKPNLHTLYWKALFDTENLKDVVAKLNGQAEIFYRKHSIKKDERTIHRANKSLQN |
| | | | | | KNENNPKKTSLFEYDIIKDRRYTVDKFQFHVPITLNFKMEKMTQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDRRNATKDQPMDTDGNGA |
| MG29 | 6410 | MG29-87 effector | Protein | Unknown | KYEHLFNRNRESRLFEWLDIRFENDHLTENEYDEIKDVLDKFDKFTTYFTGYKENRANLFVADE |
| effector | | | | | KATATAYRVVNENMPRFFENCIRMENIKKRHLDLYKLLDSFEGYFVPQAYANTICQPATTDYNKTI |
| | | | | | GRPTQNPDEKGVNSIINEYRQKNQIKNRELPMMAQLYKQLLSDRITVFLDPVINNDEEMQSIVAE |
| | | | | | TIEIARGLFSEVINLTAIHALADNSENIYINSSALANLSHRVYDDWNLIYRACEAKMIKLEGKQKK |
| | | | | | GLENKLKMAIPMSELQNIIEEYIATLDEELKLSYYKIPILCNYFQNPPLDDFESATLKFEQIVKTTT |
| | | | | | PRTDLIHAIKEVLDKAMEVVRFFKPLYLFKGRSPLEVPDRNEDFYNEFERLYAELNLISKIYDRVR |
| | | | | | NYATKKQFSQDKIKLNFNNPTLLDGWDLNKEQDNLCVILIRDGNYYLALMNRDYRRLFDLKND |
| | | | | | EVRNKALGKAGDHCYSKLEYKQVTGANKMLPKVLFAATNSDLFKPSQEILDIRKTGSHKKEAG |
| | | | | | NIEALHKWIDFCKQSIATHPEWNDHFDFKFRSTSEYSELTEFYNDFDRQAYKIKFVDIKVEYIDQL |
| | | | | | VKEGKLYLFQIYKKDFSPYSKGRPNLHTTYWRMLFGNENLANITMDTDRPIFKLNGEAEIFFRK |
| | | | | | ASLEKQITHAKGQPITNKSKKDNGKESESIFEYDLIKDKRYTEDKLFFHCPITINFRAPGTTVGSF |
| | | | | | NRKVNYFVERNPEVKIIGIDRGERHLLYYTVIDQKGNILEQGSLNQIHNSYTSAGRVVEHNINYRD |
| | | | | | LLHEKEKGREEARKNWETIENIKELKAGYLSQIVNLLSNLMIKYNAVLVLEDLNAGFKRSRIKVE |
| | | | | | KQVYQKFEKAMIDKLNYLVFKELPPGSSGHYLNGYQLTAPFTSFRDLGRQSGFLYYVYPSYTSHI |
| | | | | | CPKTGFVNLLNTRYESIEKAISFFEKFNSIKYNPGSDYFEFDFDYASFGKDVARSQWCVCTAGEK |
| | | | | | RYYYANHDKTSRECNATQQIKELLDKYNIEYIRGKDLLPEIIKKNDKGFFNGLMFLLGVVLQMR |
| | | | | | YTVSGTSNDDDFILSPVMDEQGQFFDSRSAATSEPQNADANGAYHIALKGLKIISSISDGKLKTVN |
| | | | | | KNERQDWFAYVQNKMYR |
| MG29 | 6411 | MG29-88 effector | Protein | Unknown | MKEDIKTKIDIWDDMINQYSLSKTLRFELKPIGRTLENIQYIIEEDKQRDKDFQEVKKIMDKYYS |
| effector | | | | | HFISRVLNNKIKISRDSLDEYKEIYFNLKKKIHDNDLKKQLELVQKKITKNISLMIKSDEDFKNIFG |
| | | | | | KEFVKEILKKYLEEKGNQSELELVSKFNDWTTYFTGFYDNRKNVFSDKDIPTSIIYRIVQDNLPKY |
| | | | | | LDNLYNLEQLDKYDIDFSYLNENFGGSIGNKDLKEYFGIENFNEFLSQDGIDKYNYLIGGYSKDM |
| | | | | | KDKVKGINNIIQEYSQQNKEDSDIKKLKFTSLYKQILSDKDEKISFRFFNFNSVDEMLKCIDIFYNK |
| | | | | | LNDENIFENLNFLFDDLNSEIYDKNKVFIKKNKLTFLSNELFGNYSVINYLLKEYVMKNYKDYNT |
| | | | | | EAKFEKWFKGKELLPLSLIEISIARVFDFFDSNSSEIKRFQNIVGVNFINPISFYLTQFKTEGFNLFE |
| | | | | | EIKSNYEAYRNLLSSNILDKQNLSQEEKEIKSKQVEILKSFLDSILKLNSFVDIFSTYNVKKDIVEKD |
| | | | | | NDFYNNYEELLLCISEVISIYNQVRNYITKKKTDIKKFKLNFEKSTLLDGWDVNKESANLSVVLRR |
| | | | | | NGKYFLGIMNKKDNKIFDKILPEFITSNNESFEKMEYKLLPGPNKMLPKVFFSEKNIKYFNPSQEI |
| | | | | | IDIRNYSSHSKNGEPQKGYSKKDFNLDDCHKMIDFYKESIEKHDEWKSFKFNFKPTQEYIDLSEF |
| | | | | | YSDVSSQGYKLDFLGISNKYVEKLVEDGKLYLFQIYNKDFSNYSKGKKNLHTIYWEELFSEENLK |
| | | | | | DVVYKLNGQAEIFFREKAFDAHITHPRNQDIENKDPIKNKLYSKFQYDLIKDKRYTYDKFLFHCP |
| | | | | | ITINFKAPANALRFNDRINEIIKNNSEDVKILSIDRGERHLAYYTLLNSKGEIEKQESFNLVQDKFG |
| | | | | | RNSDYHNKLNKIEGDRDKARKNWNTIENIKELKEGFLSQIIHKIAKIVVEENAIVVFEDLNFGFKR |
| | | | | | GRFKIEKQIYQKFEKMLIDKLNYLMFKDRDKSSFGGCLKAYQLTSKFDTFQKLGKQTGVIFYVN |
| | | | | | ASYTSKICPVTGFTNHIYPKYETLDKSRDLFKKFKSIKYNADKDYFEFEYMVSDFNKTLKLIKNE |
| | | | | | WKLCTFGERLITIKDKKTGYFKTESIDLTQRLKDLFESYDIDYENDFDLRDLIIKQTESDFFKSLIF |
| | | | | | YLKTLMSLRNSYTESEVKDFMRKEGDSFNLNNYDYILSPVEKDGVFFDSRNVDNLLPKDADANG |
| | | | | | AYNIGLKGLMIIDKIKNSPEVKSLNLKIEKQDYLNFVIGRNM |
| MG29 | 6412 | MG29-89 effector | Protein | Unknown | MKSTLDQFSHLYPMSKTIRFELIPQGATTANIESRGFLKKDEERAESYKKMKETIDRFHQDFIEK |
| effector | | | | | AMAHVRLSNLEDFENLYNAPNEEKKEDKYKKQLEKVQERLRKEIAKGFKSGEVKAIFLKIDKK |
| | | | | | DLVTKLLEKWIEENNLEDVHFDPEFKKFTTYFSGFHQNRKNMYTDKAQSTAIAYRLVHENLPKF |
| | | | | | IDNINIFKKVSEIPELKQNLEKLYKEIEEYLGIVSIEEAFELGYFNEVLSQKGIDVYNLILGGRSEKE |
| | | | | | NKKKIQGLNEHINLYNQKQDKKNKIPKLKVLYKQILSDRTSTSFLPDAFEDDDNSTASQKVLAAI |
| | | | | | HQFYHTQLLDYQPSDKAETINVLKSFQGLLADINNFDLDKVYLRNDKSISTIAQKMAGNYGVLR |
| | | | | | DALNYYYENKIDPEFQIKYDKATTDKKRENLDKEKSKFTRQSYISISTLQTALDTYVESFDETHDV |
| | | | | | KQVYSPTCIADYFKDHFKAEPKEGSDKEFDFVSNIEAKLSTIKGLLNTPYPENERLQQDKKKIDAI |
| | | | | | KLFLDSIMEYLHFIKPLALPEDFTLEKDEHFYTLFEEWYEQMQLLIPLYNKVRNYATQKPYSTEK |
| | | | | | FKLNFENSSFLSGWAPDYNTKGGLIIKKQDNFYLCIVEKKLKKEDVEFLKTSPEDHLAHRVIYDF |
| | | | | | QKPDNKNVPRIFIRSKGTSFAPAVQKYDLPVESIIHIYDEGLYKTDFKKENPAVFKKSLVQLIDYF |
| | | | | | KLGFSRHDSYKHYDFEWKESEEYENIADFYQDVINSSYELRDEAINFDHLHQLIDQGKLYLFQIY |
| | | | | | NKDFSKHSKGKPNLHTMYWKALFEKQNLKDVVYKLNGEAEMFYRKKSIQDKNRVVHKKNIKV |
| | | | | | ARKFYKDDKKTERVPDETVLRLNKFYKGQIQESGLKKEDLKFKDNYSLFHEQGKDIDLIKDKRY |
| | | | | | TVDKFQFHVPITLNFKAKGNDYINNEVLDYLKDNPDVNIIGLDRGERHLIYLTLIDQEGNIIEQET |
| | | | | | LNSIVNKKHAITTNYHQLLDDKERERDKARKNWGTVETIKELKEGYISQVVHKIAKMMVEHNAI |
| | | | | | VVMEDLNMGFKRGRFKVEKQVYQKLEKMLIDKLNYLVLKDKKPDEPAGIYNALQLTNKFESF |
| | | | | | QKIGKQSGFLFYVPAWNTSKIDPTTGFVNLFHVKYESVNKAKTFFSHFKSIRYNKNQGYFEFDFD |
| | | | | | YNDFTTRAEGTRSEWTVCTYGERIKTFRNPDKVNQWDNQEINLTEAFEDFFGKHEIVYGDGTDF |
| | | | | | KDQISVKDNKDFFAELIHLFRLTLQMRNSITNSEVDYLISPVKNSSGTFYDSRKADASLPKDADAN |
| | | | | | GAYHIAKKGLQWVNQIQEFDGDDWKKLKLNKTNKGWLKFVQCNT |
| MG29 | 6413 | MG29-90 effector | Protein | Unknown | MSKTLRFELKPIDDTKKIILEGFVISDQERAKDYKELKKIIDEYIIKYYIEKSLSKNNILSLYDLRNL |
| effector | | | | | ADCIKKLRSLQQLKKKQENEESIEKQQESLRKKIVQAFQLDTKEKRNRLFGKELIEEVLPEWLN |
| | | | | | SSSLKDIEHKKEIVKKFSKWTTYLTGFNENRKNMYSYKEQSTAISHRIINENLPQFLLNLNTYEKI |
| | | | | | EKNFPELETQLESLKEQLKEEFEYFNVRSIKDLFKIGFFNKCLTQKGIDNYNVIIGGKVLEGDQKI |
| | | | | | QGINEKINLFRQAQSNKNDRETIRKISNKNLPLMQTLYKQILSERESHSFYFEEFKSRNEVLESINQ |
| | | | | | YWESIFKKEGDKSILQKTENLFMNLKKYELDQIYFKNGDLPHVSNKIFEDYSVIKSALCFQAEKK |
| | | | | | FSTKKEREQYIKQDFFNFQEIQTALSGYLKENEEIQASEQFSGSEEIKKVLSADSNKGQNNILAVY |
| | | | | | FNLEFSEKQYNNKSLLGFMEELNRHFKEISLSKTQNEFDEKETQIIQNFLKPFIPIQFKKSCSYNG |
| | | | | | RETPKKQDQYNMGRGGVGLVDLLQIIKPVYLKKDRKKIMDLEKDASFYNEFEELYNKLSQVFP |
| | | | | | LYNKVRNYITTNKNQTKKIKINFEDSTLLDGWYVNKETDNLSVILRKKDKVIGWKDYLGVMNT |
| | | | | | HGMREANKIFDYHIKSGKSDKKQKEKNRLKQKVLHKEDNENFYEKMNYKQISNAAKDIQNLIKI |
| | | | | | DGRVCRKTKNLEDIKKKYLPSEIWKIKQNESYKGADKSGEDLKKFINYYKDIAVHYWKQFNLSF |
| | | | | | KDSGEYRDFKDFTDDINSQGYKVWFDKIKSSYIEDKVKAGEFLLFEIYSKDFSADSKGRPNLHTS |
| | | | | | YFKLLFEECNLKDIVYKLNGQAEIFYRKASKQKKISHRANVSIENKNPDNLKKTSKFKYDLIKDR |
| | | | | | RFTEDKYFFHVPIDLNFKARSTSPFNFNQDVLQFLKNKKDINIIGIDRGERHLAYWTVINQKGTIL |
| | | | | | EQGSFNKITTNYKGQNQKNGKMTTNYHALLEGREKERDENRKAWTKIENIKELKAGYLSHLVH |
| | | | | | QIAHLMIKHNAIVIFEDLNRGFKRGRMKFEKQVY |
| MG29 | 6414 | MG29-91 effector | Protein | Unknown | KVFSIADIETALDKEIDGANFAAANIKAEYYKKTDDIFLNYFSNRSKEILKNLLASWKNLEEYGVL |
| effector | | | | | ELKELDEKRGKEGEKGYEQIARLKTFLDNANEFLNFIKDWNVKTDKLPNNAIHDWYEKLNDFIN |
| | | | | | QFHIITLYNKVRNHVTKKPYSLDKVKINFENSTLLAGWDRNKESNNCGILLEKSGLYYLGIMTSL |
| | | | | | SNKLFDYEFSEKDTTKIRAQKLVLAEKVLDKNGTNSYRKVNYKLLPGPNKMLPKVFFAKSNSKL |
| | | | | | FNPSAKITKIKVNKLYSKENIEKYGIQNLHDYIDFCKDALCRHPEWSKAYGFTPASFRKTTQYKSI |
| | | | | | NQFYKDVETMGYSISFANVREAYINEKVENGELYLFQIYNKDFSQERKKERANRKENLHTYYWK |
| | | | | | ALFDERNLKNIVFKLNGQAEIFFRKASIKLSDEKKCKGDHCEKLKDKFNYPIIKNRRFTENKFLF |
| | | | | | HCPIGMNFKAPSIPGRENTKINTFLQNNPNINVIGIDRGEKHLLYYSVVNQDGDTIKQGSLNTIAN |
| | | | | | GFIPKGETVERCIDYHAKLDEKEKKRDLARKSWGVIENIKELKSGYLSQVVHKLAKLIIKYNAIV |
| | | | | | VLEELNQGLRRGRFKVEKQVYQKFEKALIEKLNYLVFKSEKNPLEAGHYLNAYQLINKFESFEK |
| | | | | | VGCQSGILFYTVATYTSGTDPVTGFLKNVYVKYESIEKSVKFWNSFDSIIYNVEKDRFEFTYTLGE |
| | | | | | ISSTKANTGKNESKIVKNQWTVCSSVERSRYIKPELTEEQKQILDYKGIGKLGTHELFKVTDKLK |
| | | | | | ALFDGEINYRENPKLKSRLCTIKDAAFHRKCLYYFNAIMNMRVTDSGANKGTNENDFILSPVEPF |
| | | | | | FDSRCGCKKLPENGDANGAYNIARKGICILKKLNAVPDISRPEILIDKKIWQDYAQSCETLKQQL |
| | | | | | EKMK |
| MG29 | 6415 | MG29-92 effector | Protein | Unknown | MNTSLQPNEKLVQQKDKTLLIKNYLDSIVNLLHFIKSLKPREELSQKDEAFYGRFDELYEALNVI |
| effector | | | | | TPLYNKVRNHLTKKPYSTEKYKLNFENSTLADGWDLNKEADNTTILLRKEGNYYVAVMDKKHN |
| | | | | | KIFRDIPASAKGEAVYEKMVYKLLPGANKMLPKVFFSKSRIEEFNPSTELLENYKNETHKKGESF |
| | | | | | NIQHCYNLINFFKSSICQHEDWKHFNFNFSETGTYEDLSGFYREVEHQGYKITFVNISESYIHKLV |
| | | | | | EEGKLYLFQLYNKDFSKYSKGKPNLHTLYWKMLFDETNLKDVVYKLNGEAEVFYRKKSIEDKN |
| | | | | | KIIIIKANQPISNKNPENVKKQSSFQYDITKDIIRFTKDKFQFIIVPITMNFKAKGILNVNNEANKYL |
| | | | | | RNNPDTHIIGIDRGERHLLYLTLINQQGEIIKQESLNIVANEKQKTDYHKLLEAKEGKRDEARRD |
| | | | | | WGTIENIKELKEGYLSQVVHKIAEMIVDYNAIVVMEDLNFGFKRGRQKVEKQVYQKFEKMLID |
| | | | | | KLNYLVFKTPKPNQPGLLNALQLANKFESFKKLGKQSGFIFYVPAWNTSKMDPVTGFVDFLKPK |
| | | | | | YESVDKAIAFIKQFDAIQYNKAKQYFEFVFDYSNFTEKAEGSKTKWTVCTTNTERYVWNKTLNN |
| | | | | | GKGGQEHNNVTEKLEVLFGNAGITYGNGENIISKITEQTGAEFYKALLKLLSITVSLRHNNGEEG |
| | | | | | EKEQDYILSPVAPFFDSRKATDTLPKNADANGAYHIAKKGLWVLEQINKCEDFKKLKLAISNKE |
| | | | | | WLEFVQKSSIKTNYPQPFA |
| MG29 | 6416 | MG29-93 effector | Protein | Unknown | MNTTVFTNQYPLSKTLKFELIPQGKTLENIQHEGLLEQDNNRAVSYQKIKRLIDEYHKVFIENSL |
| effector | | | | | KGMKLVELEVYSEWYQKKDKVDADKKAFEKVKEALRKQISLTFSSQDIYKTLFSKELIKEDLLA |
| | | | | | FVKQEDKSLVNEFKDFTTYFTGFHENRKNMYVADEKSTAIAYRLINENLPKFIDNLNIFLQIEDKS |
| | | | | | VSLIADFNKALQEMEEIAQGRTLEDIFTLSFFNETVTQHGIELYNIIIGGRTGENGKSKIKGINEYI |
| | | | | | NLYNQQQNNKLNRLPKFKQLYKQILSDRNSVSFVLDNFENDNHLLECIEQFYQSGICHFESDGNS |
| | | | | | IDLLTTIRQFLNGLDSFDQTKIYIRNDNSITDISQKIFGDWNVIRAALTAFYEKTNPIKPRERLDKF |
| | | | | | EERKESWVSKTGYFDINTIQTALDSYDSEAIIDKYSPDCIVKHLGNIGKSKESTIDLVDTIQQNYAV |
| | | | | | IKDLLNNPYPEDEKLGTAKELVAQIKTFLDSLLNLIHFLKPLNVGNDELEKDESFYSIFTPLYEQL |
| | | | | | SEVIPLYNKVRNYLTQKPYSINKVKLNFENSTLLNGWDVNKEVDNSGILFRKNGLYYLGIMDKN |
| | | | | | NNKIFDRNVPQCQDLTTSFEKINYKLLPGANKMLPKVFLSKKGVETYNPTLENIENYNNETHKKG |
| | | | | | DTFNIDDMRKLIDYFKESISLHPDWKHFEHKFSETKSYEDLSGFYREVEKQGYKISFKNIDEAYIN |
| | | | | | KLVEEGKLYLFQIYNKDFSPFSKGTPNMHTLYWKMLFDISNLKNVVYKLNGEAEVFYRKSSIKE |
| | | | | | KNMIVHKANEPLQSKNELNTKKESSFTYDIIKDKRFTLDKFQFHVPITMNFKATGTDNINPSVNQ |
| | | | | | FLQNNKDVNIIGLDRGERHLIYYTLVNQKGEILEQGSLNEISNEKQKVNYKDLLVKKEGDRTQA |
| | | | | | RKDWNTIENIKEIKEGYLSQVVHKIATLMVERNAIVVMEDLNMGFMRGRQKVERQVYQKLEK |
| | | | | | MMIDKLNYLVFKTKATDEPGGVLKALQLTSKFESFKSMGKQSGFLFYVPAWNTSKIDPTTGFVD |
| | | | | | FLKPKYESIDKAKSFFSSFKAIRFNASSNYFEFEFDYNDFNGKAEDTQTNWTVCTVGTERYSWNK |
| | | | | | KLNMGKGAIEKIDITQALQLLLDEANILYAAGNNLIQDINDQNNADFFKKLIKLLSVTLSLSHSNG |
| | | | | | LSGEAEKDFILSPVKNDQGQFFNSNDFDGRLPKDADANGAFHIALKGLWVLRQINEAEDMKKVK |
| | | | | | LSITNKEWLQFVQNKHYKS |
| MG29 | 6417 | MG29-94 effector | Protein | Unknown | MNENNSIWDNFINKYNLSKTIRFELKPVGKTIDFIKENGLIEEDKQREKDFNEVKKIMDEYYVEFI |
| effector | | | | | ENCLKNIKLDLSDLQEYYTIYFELKKDKYNSDLKKQFKNIQKKIANNMYQQIKDVDNFNNIFDEK |
| | | | | | FVNVVLPKWLKEKGREQDEMLLSKFKKWTTYFDGFFNNRQNVLSNDLIPTSIFYRIVVDNLPIFL |
| | | | | | DNIAKYDKLKKINGFPLESIEKNFAKYLNNVSLDYFFSLENFNNLLNQEGIDLENLILGGYVENNS |
| | | | | | KICGLNESINLYSQKPENKETSKQLKQLIMMPLYKQILTEKKSFSEKFGIIQDNQELVNLIDDIYT |
| | | | | | NNYVLNFDNLQKLIKNLNEYDLNQIYVNTLSLQKISKSIFKDLFVIRNGLKEFIKNKEHIKSDKKS |
| | | | | | EKKLEEILNQKYFSIFEIQEGVKLLKLSKEGNSFSDNFMIDYFLENINDKLFETITNNYNQFNSITV |
| | | | | | NLLNKIRLEDIPTIKDLLDSLKKLFDNIKPLYVNLNMGTNTKIQEAYNLDSLFYIEFNKIYIAFSVII |
| | | | | | PTYNKVRNYVTKKQKNVKKFKLNFNCPVLLKGWDVTKEPENHSVLFRKDGDYYLGIMPKGHT |
| | | | | | HMFSNIENIDNDGEYYEKMVYKQISDASKDIQNLFVKDNKTQRIVGRKEKEGDNKGKNVELENA |
| | | | | | RKKYLPEDIQEIKKKKSYLRSSPSFNEADKNRFIDYYKERVIDYENYNMFNFKFKESEEYLDFNDF |
| | | | | | IKDVDNQGYKIEFIKINEQFIMDSVNTGKLYLFQIYNKDFSKNKCESNKNSKPNLHTIYWEELFSE |
| | | | | | ENLKDVIYKLNGEGEIFYRECSKAIPKEVTHPKNIPITNKNPINNKQTSIFPYDLIKDKRYTEDKFL |
| | | | | | FHCPITINFKQNNMLKGFNQQIISYLKENNVTILSIDRGERHLLYYTLLDLQGNIIKSGSLNLVSDD |
| | | | | | VKRQWNYHDLLDQREKERDKARKDWMPIEAIKNLKEGYLSKIIHDISKMVIENNSIIILEDLNFG |
| | | | | | FKNGRFKIEKQVYQKFEKMLIDKLNYLVFKDKPKTEVGGSLKGYQLTDKFVTFNRLKKQSGILF |
| | | | | | YVDAKYTSAIDPTTGFFDMIYPSYTSVDNSIALFKKFNYIKYNTKEDLFEFNFNFSKFNTELKLYK |
| | | | | | DNWSIWSNGVKLVNYRNPNKNNEWETKEVIVNNQLKKLFGDYKINYDNDEDLIPQIISNTTADF |
| | | | | | HKQLIENLKLVLKLRNSRINSDDDYILSCVKNKDGVFFDSRESSNNLPKNGDENGAYNIGIKGIIL |
| | | | | | LNKIRENKEIKKITKEEYINYLIKRGK |
| MG29 | 6418 | MG29-95 effector | Protein | Unknown | MKLNKFTHQYPISKTLRFELKPVGETADYIEDFKSQYLKDIVIQDQQRAEDYESIKVIIDEYHRHY |
| effector | | | | | IEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSHEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNDLLKSL |
| | | | | | GKFIKEMVKEDGLFKKLEDSIKLITDADLHRTFIKNGVEITKISQSIFGNYSILKSAIYHHAESVLY |
| | | | | | PDPISGKISEALKEKRIKYVNKQVVFSIAELETILSNYASQMTDDNPDKEIIAQCENSEHPIRTYFL |
| | | | | | NAIENVKNDKDIEFGKAIENVLPLISLENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEEGKLFLFQIY |
| | | | | | NKDFSPYSKGKPNLHTLYWKALFDTENLKDVVAKLNGQAEIFYRKHSIKKDERTIHRANKSLQN |
| | | | | | KNENNSKKTSLFEYDIIKDRRYTVDKFQFHVPITLNFKMEKMTQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDSRNATKDQPMDADGNGAYHIALKGLWNLEQIRNWDGESRLNLAMKNVDWFSFAY |
| | | | | | QKPFKK |
| MG29 | 6419 | MG29-96 effector | Protein | Unknown | MLQNFTNQYQLSKTLRFELRPVGKTKEHIEAKGLIIQDEQRAEEYKEMKKIIDRYHKAFIEDALN |
| effector | | | | | GIAIEGLETYEKLYFAIKDEKGKKEFEKLQDTLRKRIVELFKKHPKWSTLFKKELIRNELLTFLD |
| | | | | | SEEIPEEQKINEKEIVLKFMDFTTYFTGFHENRANMYIDEALHTAVAYRIVHENLPVFLGNKKTF |
| | | | | | EQIATKYPELIADSKDAIESHLFGAVFEDMFTLAYFSHTLAQNHIDLYNTMIGGKVSNDGSKIQGF |
| | | | | | NEKINLYRQKHGLSKRDLPNLKPLYKQILSDRESLSWLPEAFEDKNELAEAIKTFYQNNIIAFECC |
| | | | | | DGKVNLLEKFPEIFKENQYYDLSKIFIKNDKSLTDIAQAIFGKYGVIKEALWEKHLRDNPKAAKS |
| | | | | | KDISADEERFFNKKDTYFSINDIHIALKAGEDRVQTGLDFSINDIHIALKEAQSPSDILTYFSNEIKP |
| | | | | | LTKAVQVAYKTWLDDQEKTERIKEVMDALLAWQRFLKPLSVKSDVDRDIAFYATFESYFESLSA |
| | | | | | VVKLYDKVRNFMTKKPYSLEKFKLNFENSTLLDGWDVNKETDNTAILLEKNGLFYLGIMDKKH |
| | | | | | NRVFKNTPESQDDSSYRKINYKLLPGANKMLPKVFFSNSRIDEFAPSSDIISNYKKGTHKKGEIFD |
| | | | | | LEHCHKLIDFFKSSIQKHEDWKNFEFKFSDTASYKDLSGFYREVEQQGYKITYKNISQSYIDTLVS |
| | | | | | EGKLYLFQIYNKDFSPYSKGTPNMHTLYWRALFDEKNLADVVYKLNGQAEVFYRKKSIIYSDEV |
| | | | | | MQKGHHAKELVGKFDYPIIKDRRFAFDKFQFHVPITLNFKAQGYTNLNAIVNEMIASGKEDIKII |
| | | | | | GIDRGERHLLYLSLIDAQGKIVEQYTLNQIINSYNGKDHVINYHEKLAKKEDERAKARVNWGTV |
| | | | | | ENIKELKEGYMSHVIHRIATLMVEHRAIVVLEDLNFGFKRGRFKVEKQVYQKFEKMLIDKLNYL |
| | | | | | VDKKKSPNELGGVLNALQLINKFESFEKMGKQNGFLFYVPAWNTSKIDPVTGFVNLFDTRYASV |
| | | | | | EKSKEFFGKFKSIRYNSEKDYYEFEIDNYTQFNPKAEGTRQNWTICTYGDRVLTYRNSEKLNQW |
| | | | | | DNKTVQLTDGFKTLLNGQADNLKEYIVAQNDKAFFEKLLGLFRLTLQMRNSITGTDIDYLVSPV |
| | | | | | VDENGVFYDSRVCDDTLPKDADANGAYNIARKGLMVVKKIKEAQDIKKPDLKITNKEWLQFAQ |
| | | | | | R |
| MG29 | 6420 | MG29-97 effector | Protein | Unknown | MDLKNNNLSEFSNQYQLSKTLRFELKPQGKTIEHIKKKGLISKDEARAKSYEKMKKTIDGFHKY |
| effector | | | | | FIEVAMAQVNLSFLDEFEQLYNAPTEEKKDDTYKKAFEKVQNNLRKEIVRGFKVGEAKDIFSKID |
| | | | | | KKELITKLLEEWISKQQNDDIYFDEKFKTFTTYFGGFHENRKNMYTDKAHTTAIAYRLIHENLP |
| | | | | | KFIDNIKIFEKVKTVPEVYKNCSTLYTNIEAYLNINSIDEAFELAYYNEVLSQKEIDVYNLIIGGQT |
| | | | | | KEEGKKKIQGLNEYINLYNQKQEKKNRIPKLKPLYKQILSDRENISFLPELFEESQDVLDAIESYY |
| | | | | | KANLVDYKPDDKDDTENILKELYSLLKGVNSFDTNKIYIRNDKSLTDISKAIFGDWAIIDAALEFE |
| | | | | | YIQDITIPKSGLTKKQEKEKERYLKQPYFTIQEIEQALKAYRNENEVLADFTEGMVGNYFYSHFK |
| | | | | | TKTDSDKEFDFVSNIEAKYSCIKGVLNTKYPKNQKLNQDKVTVNNIKVFLDSLMELLHFIKPLAL |
| | | | | | PNDVPFDKDETFYGHFETYYEQLQLLIPLYNKVRNYATQKAYSKEKFKLNFENSTLLDGWDVNK |
| | | | | | EEANSCVLFQKEGLYYLGIMDKNHNKVFRNLPSTSSKNTYNKINYKLLPGASKMLPKVFFSSKNI |
| | | | | | AYYNPDKEILRIRNHSTHTKGGSPQKGYEKQDFNVKDCRKMIDFFKTSIHKHPDWKKFGFEFSE |
| | | | | | TAHYNSIDTFYREVESQGYTITHTAIDNDYIDKLVNEGKLYLFQIYNKDFSPYSKGKPNMHTLYW |
| | | | | | KALFEPDNLNDVVYKLNGQAEVFYRKKSIKEKHKVIHKAQEPIINKNPLAQKKTSVEDYDIIKDK |
| | | | | | RYTVDKFQFHVPITLNFKAVGNEYLNTNVLDYLRDNSDVKIIGLDRGERHLIYLSVIDQEGNILEQ |
| | | | | | ESLNTIINKEHNIHTPYHNLLDKREKERDSSRKEWGTIATIKELKEGYISQVVHRITQLMVKHNAI |
| | | | | | VVMEDLNFGFKRGRFKVEKQVYQKLEKMLIDKLNY |
| MG29 | 6421 | MG29-98 effector | Protein | Unknown | MLKQFTNKYQLSKTLRFELIPVGKTKDILDKKGLVLEDEKRSEEYKIVKTLIDDYHRYFIQEALS |
| effector | | | | | RKNLLDLEKFENLFLKKDKNKDEEEDFNKIKTDLRKQIVSFFESNELFKNIDKKELIKVDLLNFL |
| | | | | | TDENDKNIVKKFSDFTTYFTGFHENRKNIYSADEKASSIAYRVIHENLPIFISNKVAFLNILKEYPEI |
| | | | | | IKNTQKNLENHLLGAMVEDMFNLENFSLTLTQIYIDIYNTILGGKTLEDGTKIQGLNELINLYRQ |
| | | | | | KNSIEKRKLPNLKPLHKQILSDKETMSWILEAFTKHEEIETAIEKLYHDNIVDFYCCDKRINILDE |
| | | | | | FENLFSKNDDYDLTKIFIKNDLSITSISQDIFKDYKIIKDALWQKYLNDNPKIAKSKDLSADEEKYF |
| | | | | | SRKNSYFSFFNILEALKFVEIKLDFDLFEYFKNIVKEKSDLIKVSYFEWEKDKNNKKSTKDLLDNI |
| | | | | | LNLQRAIKPIYVKSELDKDIVFYAMFDTYFESLNKIVKLYDMVRNFESKKPYSIEKFKLNFENSTL |
| | | | | | LNGWDLNKETDNTSLLFEKDGLYYLGIMNKKHNKSFLNKKESEENNCYKKIEYKLLPGANKML |
| | | | | | PKVFFSNKNIDFYSPSDNLLENYKNGIHKKGTTFDLNFCHELIDFFKSSINKHPDWKNFNFQFSNT |
| | | | | | LEYNDISDFYREVEQQGYKISFKNIEIKFINNLINEEKLYLFQIYNKDFSPYSKGTPNLHTLYWKM |
| | | | | | VFDKENLKNVVYKLNGQAEIFYRKKSIEYSDDKLNKGHHNEDLKDKFNYPIIKDRRFTIDKFQFH |
| | | | | | VPITINFKALGKNNINNDVNEFIKQNHKDIKIIGIDRGERHLLYLSLIDNNGKIIEQYSLNEIVNNYN |
| | | | | | NQEFKVDYQDLLDKKEKDRAFARENWGIIENIKELKEGYISQVIHKIAKLIVEHNAIVVLEDLNF |
| | | | | | GFKRGRFKVEKQVYQKFEKMLIDKLNYLVDKKITNDEIGGVLNALQLTSKFESFERLGKQSGFL |
| | | | | | FYVPAWNTSNIDPATGFVNFFDTKYQSVEKAKEFFSKFDYIKYNIKKDYFEFAFDYNNFTQKAKD |
| | | | | | TKTKWVLCTYGTRIKTFRNKEKNNQWDNIEVDLTTEFKKLLQATENKDLKEFITSQNTKDFFEQ |
| | | | | | LLYLFRLTLQMRNSITNSKIDYIVSPVADKNDIFYDSRNLSIALPKDADANGAYNIARKGLMIVEKI |
| | | | | | KKSDNNKIDMKITNKEWLQFVQGK |
| MG29 | 6422 | MG29-99 effector | Protein | Unknown | LRNHIANCFKDEKELTIVGKDFIKKILPQWLEDSGSLEYKKLIDEFKDRVTYFTGFDNTRKNIYT |
| effector | | | | | NQAIATSVAYRVVHDNLPIFIDNLQRFKKLQDYREQIDLVQIERDLKVNINEMMQLDYFNQCLTQ |
| | | | | | TGIDLYHQLLGGIEAKEGVEKVQGLNERVNLAQQQLNKNSIDANSKQTKIKLPQLVTLQKQILS |
| | | | | | EKRSGAFANPIDNDRELCSLLDKVVYKNDRLLVSIDKTNTDTGEIKQQQVDVNQLMTNTIDALN |
| | | | | | HADLEQVYIHSDMLNMLSQRKLGVWSFLKEEVWQPYIDSQYSKKTEREKVQKMPLISIADLHNS |
| | | | | | INYFLKQNEGHDAFNNENQQHTALAKLYHWYNDSGEKPLVTYLSNQWQLLNIGQGKEVKSLSQ |
| | | | | | LHQALTPILQKYGTSLEEKLKSNKEAVRQIKEYLDALTDLQHLVKTYQFKVTKKDAKNVAIKAL |
| | | | | | LQDSHFYNHYQQLLDAVSMIVPIYNRVRNYLTKKPYSIEKYKLNFENGTLADGWDVNKESANFT |
| | | | | | VILRKKNDRNKYDYFLAIMPTGKGLNRAFEAGNLATTKNKDGYEKLFYKYLPGPNKSLPKSFLS |
| | | | | | QKGRTNYKPSEELLENYKNKTHTKGDAFNLQHCRDLIDYFKKCIQKHPDWGVFNFTFSNTDSYE |
| | | | | | DISHFYREIEQQGYKVWFENIAQQYIDNLVSAGKLYLFQLYNKDFSPYSTGKPNLHTLYWLSLFS |
| | | | | | SQNIADPIYKLNGEAELFYRPKSIEKRNVTIHRKNQPIKHKIGVPQNSQFGHDIIKDKRFTENYYQ |
| | | | | | FHVPITINRASVTDKNLNDAINQYSCDSPNLTILGIDRGERHLAYFTLIDQNKKILEQGSFNKPNG |
| | | | | | GQDYQKLLDKKEKGRGEARKSWSTIENIKELKAGYLSQVIHKICQMVIEHNAIVILEDLNFGFKR |
| | | | | | GRFKIEKQVYQKLEKMLIDKLNHLVFKDKAVDEAGGLRNGYQLTAPFTTFEKLGKQTGILYYV |
| | | | | | PAYHTSKVCPKTGFVDLLYPKYENLTKAKDFFSKFDFIRWNQQNNYFEIGLDYNKFKAKKDITG |
| | | | | | LRTKWTICTYGDRIHQTKGKVSQQWESRDINLNKQWQTKLEESNIAYTHGNCIVSDIVAKDSIEF |
| | | | | | WRSLFWVLKMTLQMRNSKPNSTDAKDDYLLSPIAYQPNQFFDSRKASDNEPKDSDANGAYHIAL |
| | | | | | KGLMMLEQRKQQVKKLDFSNKAWYQYIQNQ |
| MG29 | 6423 | MG29-100 effector | Protein | Unknown | MDLKNNNLSEFSNQYQLSKTLRFELKPQGKTIEHIKKKGLISKDEARAKSYEKMKKTIDGFHKY |
| effector | | | | | FIEVAMAQVNLSFLDEFEQLYNAPTEEKKDDTYKKAFEKVQNNLRKEIVRGFKVGEAKDIFSKID |
| | | | | | KKELITKLLEEWISKQQNDDIYFDEKFKTFTTYFGGFHENRKNMYTDKAHTTAIAYRLIHENLP |
| | | | | | KFIDNIKIFEKVKTVPEVYKNCSTLYTNIEAYLNINSIDEAFELAYYNEVLSQKEIDVYNLIIGGQT |
| | | | | | KEEGKKKIQGLNEYINLYNQKQEKKNRIPKLKPLYKQILSDRENISFLPELFEESQDVLDAIESYY |
| | | | | | KANLVDYKPDDKDDTENILKELYSLLKGVNSFDTNKIYIRNDKSLTDISKAIFGDWALIDAALEFE |
| | | | | | YIQDITIPKSGLTKKQEKEKERYLKQPYFTIQEIEQALKAYRNENEVLADFTEGMVGNYFYSHFK |
| | | | | | TKTDSDKEFDFVSNIEAKYSCIKGVLNTKYPKNQKLNQDKVTVNNIKVFLDSLMELLHFIKPLAL |
| | | | | | PNDVPFDKDETFYGHFETYYEQLQLLIPLYNKVRNYATQKAYSKEKFKLNFENSTLLDGWDVNK |
| | | | | | EEANSCVLFQKEGLYYLGIMDKNHNKVFRNLPSTSSKNTYNKINYKLLPGASKMLPKVFFSSKNI |
| | | | | | AYYNPDKEILRIRNHSTHTKGGSPQKGYEKQDFNVKDCRKMIDFFKTSIHKHPDWKKFGFEFSE |
| | | | | | TAHYNSIDTFYREVESQGYTITHTAIDNDYIDKLVNEGKLYLFQIYNKDFSPYSKGKPNMHTLYW |
| | | | | | KALFEPDNLNDVVYKLNGQAEVFYRKKSIKEKHKVIHKAQEPIINKNPLAQKKTSVFDYDIIKDK |
| | | | | | RYTVDKFQFHVPITLNFKAVGNEYLNTNVLDYLRDNSDVKIIGLDRGERHLIYLSVIDQEGNILEQ |
| | | | | | ESLNTIINKEHNIHTPYHKLLDKREKERDSSRKEWGTIATIKELKEGYISQVVHRITQLMVKHNAI |
| | | | | | VVMEDLNFGFKRGRFKVEKQVYQKLEKMLIDKLNYLVFKDKLPNEAGGLYNALQLTN |
| MG29 | 6424 | MG29-101 effector | Protein | Unknown | FLKPLHIEDGNKKDNKLQQALELDADFYNNFNELYSKLKEVIPLYNKVRNYITQKPFSTKKFKLN |
| effector | | | | | FQNSTLLLGWDENKETSNWSVLFRKDGKYYLGIMAKGNNKIFENIGSKETNKSDGFYEKVRYK |
| | | | | | LLPGPNKMLPKVFFSKRNIESFEPSEEIKSIRNHSSHTKNGKPKEGFEKKEFNLRDCRKMIDFYKR |
| | | | | | SLKKHPEWNEFDFNFKETKKYEDLSEFYRDVSNQGYKITFEKISEPYIRKLVDEGKLYLFKIWNK |
| | | | | | DFSKYSKGKPNLHTIYWKMLFDENNLKDVVFKLNGEAELFFREKSISRKITHPKNKSIKNKDPFQ |
| | | | | | GKKESKFNYDIVKNRRYTEDKFLFHCPITINFKSKEQSWEINNQINKKIKKSSDDIYILGIDRGERH |
| | | | | | LAHFTLLNPKGDLVLQESFNIIYDDLKRKRNYHKKLDKLEGSRTEARKNWKKIANIKEMKQGY |
| | | | | | LSQVIYKIYNMAIDKNAVIILEDLNFGFKRGRFKIEKQIYQKFEKMLIDKLNFLVFKDMGKKNVG |
| | | | | | GPLRAYQLTRKFESFQKLGKQSGIIFYVPANYTSKICPMTGFVNLLYPKYENVRKSKNFFKNFKF |
| | | | | | IRYNPDENLFEFNFNYSDFKGKNKSEAELKRDNWSIYSFGKRLFNKKKKNSREFETQEIDPTAEL |
| | | | | | KKLFMDNGIDFEDGNNLRGKICSSDDAKFFKSLTFLLKRVLQLRNSRTGGGEDYILSCVKDKHG |
| | | | | | NFFDSRKAENGQPQDADANGAYHIGLKGLMLMKRIKDSDIDRKIDMRIKRNDFINFVIDRNK |
| MG29 | 6425 | MG29-102 effector | Protein | Unknown | MKQKSVWNNFTNQYSLSKTLRFELKPIGDTLQNIKEKGIIEEDKEREKRFNEIKQLMDEYYKDFI |
| effector | | | | | ERALKTVHIDQKDLKAYQEVYLTLKKSKQKKESYEKLEKKYLANQKLLRNEIYKKIKEFKEFN |
| | | | | | YLFKKEFVNKLLPEWLDEKKRFAEANKVREFGRWVTYFTGFFENRKNVFSNEPIATALIYRIVH |
| | | | | | DNFPKFLDNIERYEILKEHKVSFKEVEKNFKNELNGSLDAFFSISTFNDCLNQEGIDRENMIIGGKS |
| | | | | | TEDGHIIKGINQVVNEFSQKHDEKKIRKLKMMPLFKQILSDRESASFIPDKFKNDKELIDSIITYYK |
| | | | | | NQDFNKLKNVLSRLHECDLNQIYIRNDGRLNRLSKELLGDWEFINNGLKEYARSELNKNTDKKL |
| | | | | | DDWFKKEFFAIAEINEGIKRINPDISLKEYFASLKREDKNIVQDIKKNYKEFKKIEVTSDSSLLSKK |
| | | | | | REKDIETIKAFLDSIMDLFHFIKPLYMNSDNAHDIDSDFYIKENKSYEELQEIIPLYNKVRNYVTQK |
| | | | | | PFSTKKFKLNFQCSSLLGGWSSSFTTNAGLILRKDGNFFLAIVPRSLDKDTVNRLKQKGNTIDVL |
| | | | | | NYDFQKPDNKNIPRLFIRSKGENFAPAVQKYNLPINDVLKIYDKGYFRTEYREKDEKVYRESLKR |
| | | | | | LIDYFKEGFRKHESYKHYTFEWKKTEDYKDISEFYHDTEKSCYKPRFYQVNLNVLEEMVSAGKI |
| | | | | | YLFQIWNKDFSTYSKGRKNLHTLYWQELFSSENLKDVVYKLNGEAELFFREKSLEKKITHPKKK |
| | | | | | PIDKEPRKDDKGNLKEKSLFEYDLIKDRRYTEDKFFFHCPITMNFKAGNNKWIVNQRVRDAINS |
| | | | | | GVNVLSIDRGERHLAYYTLLDPKGKILEQGSFNRISDDLGRGKDYHDKLNKLEKSRDKARKNW |
| | | | | | KEIANIKELKEGYLSQIVHKITKMAIEKNAIIVLEDLNFGFKRGRFKIEKQVYQKFEKMLIDKLNY |
| | | | | | LVFKDQKKEEPGGLLKAYQLTSPLESFKKLGKQSGIIFYVPAQYTSKVCPRTGFVNLLKVKYENI |
| | | | | | EKTKNFFRRFETIYYDKNNDYFVFEFKYSDFIDEMKKKKLVRDKWTVCSYGKRLVNRQSSKHK |
| | | | | | GFETAEMDPTKELKTLFNQYELDFKDGNNLVSQICGYQDSKFFKDLIYFFRCILQLRNSRTNDSE |
| | | | | | DYLLSCVPDKKGYFFDSRNAKTAKTDEPHDADANGAYHIGIKGLMLIDRIRSGGKQDFKIDRND |
| | | | | | YFNYIMKNGGKH |
| MG29 | 6426 | MG29-103 effector | Protein | Unknown | MAENRCVLDNLTNMYSLSKTLRFELKPIGKTLEYIEKKRLISEDEKRAESYKKMKKIIDGYHKH |
| effector | | | | | FIEIAMSQVELTKLEEFAELYNASPERKKEDDFKTKFKKVQKDLREEVVAGFKNAKEEVHSSKN |
| | | | | | DKKDTKDKEEVEKSEKSDSSKNDKKDTKGKTFFEKLFMKQLFTELLDDWVKNPEDKKLVDEFK |
| | | | | | TFTTYFRGFFENRKNLYTNKEQSTAIAYRLIHENLPKFLDNIKTFEKIKSIPELYNKCDILYGEIKE |
| | | | | | YLGITCIDDVFELDYYNNILTQKQIDVYNLIIGGRVTEKGQNKIQGLNEYINLYNQKQDKKDKIP |
| | | | | | KLKQLYKQILSDKKSASFLPEAFEESQEVLDSIRDYYQSNLIDFKPDDKEDTENVLKEIRDLLAHL |
| | | | | | KNYDFNKVYIRNDQSLTNISKKLFDDWGIIKSALEFAFLQTLKIGKKGLSKKQEKEKKNYQDQS |
| | | | | | YFSIAEIESALLAYKDETEILKYLKEDTNPVANYFHTYFKATKKDAEKEFDLIANIEAKYSCIKGIL |
| | | | | | NTEYPKDKRLYQDKKLIDDIKAFLDSLMELLHFVKPLSLPSDSPLEKDELFYGQFQIWYDQLQLL |
| | | | | | IPLYNKVRNYATQKPYSTKKFKLNFENVQLLGGWDANKESDNTSVLFRKNRLYYLGIMDKKHN |
| | | | | | KIFKNIPDASPGELVYEKMVYKLLPGAKKMLPKVFFSTSRIKEFAPSEELQKKYHAGTHKKGEE |
| | | | | | FNINDCHNLIDFFKESIAKHEDWKNFEFKFSDTSSYENMAEFYREVEHQGYKITFQNISEDYINQL |
| | | | | | INEGKLYLFQIWNKDFSTHSNGKPNLHTMYWKALFDPKNLKDVVYKLNGQAEIFYRRRSISKER |
| | | | | | RIIHKANEPIENKNPNNPNKNSVFEYDIIKDKRFTVDKFKFHVPITLNFKASGKENINEEVYKYLK |
| | | | | | NNPNVNIIGIDRGERNLIYVSLINQKGEILEQFSLNDIITSYKNWENKDVEVKTQYHALLDKREGE |
| | | | | | RAKARENWGTIETIKELKEGYISQVIHKIAQMMVKHNAIVVMEDLNFGFKRGRFKVEKQIYQKL |
| | | | | | EKKLIDKLNYLVFKDKTDNEIGGLYHALQLTNKFVSFQKLGKQSGFLFYVPAWNTSKIDPTTGF |
| | | | | | VNLFNTRYENLEKAKTFFETFEDIRYNTTKDYFEFEVKEYSKFNSKADDTRQDWIICTYGERIKT |
| | | | | | FRNPDKNNQWDNKEIILSEEFKKLFESHHIDYKTNLKAQIISQENKKFFEELLHLFKLTVQLRNSI |
| | | | | | TKSEVDYLISPVMNSTGQFYDSRKAGKKLPKDADANGAYHIAKKGLWILQQINVHNEDRKKPNL |
| | | | | | AIKNKEWLQFVQSNR |
| MG29 | 6427 | MG29-104 effector | Protein | Unknown | MLSDVKTSSFVPDFFENDNELLTSLDEYIHYGNKRVKDLGDIIEKLSNFDSSEISVNGKHLSSLSLA |
| effector | | | | | MFSNYRAMSDILHAWAEENLPTVKARKNFCKKESFTLYELEAAILFYKLHMEDGDTLEESFGSL |
| | | | | | ESDKPLVSFMVRFFNEKRNAIEGLIPDAKTVIDNGSISKNRRSPKKNGKGGGKGFYQMEAIRGM |
| | | | | | LDAFSEVYRSVKWLHLIKGTDLIEGANTDSDFYEIFAPLYEEFSDTTIVLYNKTRSYLTQKPYSKD |
| | | | | | KMLVNFDAPQLLSGWDINKQKESLGVILKRDEEFYLAIMRKEHNTVFLEAPVAEEGEEFYERLG |
| | | | | | YKLLPSPSKMLPKVFFSRKWLEANNVPEDILDIKRSKSYHRASKDFSLEDCHRLIDFFKANINNYK |
| | | | | | VNESDEFGWDVFSFEFSPTSSYQDIDEFYQEVEQQGYKMWFDNISKEYVDECVKNGEIYLFRIMN |
| | | | | | KDFRPASKGKPNLHTLYWLAIFDDENLENPVIKLNGGAKMYFREHSIEEDDRIIHPANEPIANKTP |
| | | | | | ENPKKESKFGYDIVKDRRFTEDKFKLHVPVTINCKAANKITAELFNRNINKALALQEGMHVIGID |
| | | | | | RGERNLLYYVIIDWKGRIIKQGSLNCLETSQGYSIDYKAKLQRIEKDRDASRKGWATIDDIKNLK |
| | | | | | SGYMSHIVYTISRMMIEYNAIVCLEDLNMGFKHSRKSVERQVYQKFEEALINKLNYLVFKERGR |
| | | | | | SESGGYAAGYQLTAPFVSFDKLGKQSGVLFYVNPSYTSKTDPLTGFVNMLRPKYINMKKAKAFL |
| | | | | | KTFDHIKFNDEAGYFEFHADFKKFVKKVNGIRTKWVICTHGDERYVNRRDSSGRFRSAKVNVTE |
| | | | | | EMKALLHEYGIDYHDEESLQKSILNVDNSKFYRYFLHLVSVVLNMRYSVTGNDDIDFILSPVANA |
| | | | | | NGVFFDSRNADDDMPKDADANGAYHIAMKGLWSLQQIRNHDWDADDNKRLNLMMSNEEWVR |
| | | | | | FIQEAAMRR |
| MG29 | 6428 | MG29-105 effector | Protein | Unknown | LTQPGIDLYNKMIGGESLENGKKIQGFNEKVNLFRQANKLDGKSVPMIKQLRKQILGDKNTPE |
| effector | | | | | WITEGFKDKDSMNEAIVKFIKNIEHANDNLTGELFIRSKPYDYNKIFIKNRFITNISHELFKDWNL |
| | | | | | LKNNMLEQYKSKNPKSKNPEKEFAKIPYFSIAEIQESIPNQNKHFPDFIIDHFHDKIMTLIPTEKKI |
| | | | | | HELWKGNKDSIPALKNLMDYYLELYRISKPFDVDCADKDPVFYELFDEIFNDFSKVVKLYNEVR |
| | | | | | NFITKKPYSLEKIKLNFGNSTLLSGWDINKETDNTAVLLQKDGNYYLAIMNKDHNKIFKNTPVTK |
| | | | | | DEKNSYKKMEYKLLPKSYMMLPKVFFTKGNKDKYEPSEENRIYEEKTFKTGDNFNINDLHKLID |
| | | | | | FYKESVKKNPEWSCYNFNFKPTKEYQKINEFYEETDTQGYNITFKNIPAEYIDNLVKKGQLYLFK |
| | | | | | IYSKDFSSYSKGTPNLHTLYFKMLFDDRNLKNTVYKLNGGAEIFYRKKSVTYPKNIMEKGHHAE |
| | | | | | DLKGKFNYPIIKDRRFTFDKFQFNISITLNPNAQGHGNINDICRNLIKSNNTNIIGIHRAENHLIYIT |
| | | | | | VLNSSGDIIEQRSLNEIKSYGDKTVNYMEKLTERGKERNDARINWETIGNIKELKEGYLSNVISEI |
| | | | | | ANIMIKYNAICVMEDLNYDFIRERTAIEKQIYQKFEKMLIDKLNFYVDKKKSQEETSGILKPLQL |
| | | | | | ANKFTTFEKIGKESGMIFYVSPYKITDIDPVTGFVNLFDTRYFNIEKAVQFFDKFDDIYYNAKTDL |
| | | | | | FGFNFDYDNFINKEKIQNIKTKWTVYSYGERIERFNNNNIPKFKKIDLTKEFKNLFSEYSINYNSNL |
| | | | | | KKSIISLKDKNFFMKLLSLFRLTVQMRNGDFIISPVNDNLGNFFDSRNSDNKTLPENTAANGAYNI |
| | | | | | ARKGLILLNRIKQTSDIKKADLKISGDEWLQYAQDENSYI |
| MG29 | 6429 | MG29-106 effector | Protein | Unknown | MNNKLTAFTGKYQLSKTLRFELKPVGKTLEHIQAKGLISQDEARAASYQKMKKTIDGFHKHFIE |
| effector | | | | | LAMSTVKLTKLEAFQELFNASPERKKEESYKKELEKVQDSLRKEIVKGFSTGEAKEIFAKIDKKE |
| | | | | | LITELLEQWMQRQNEDDFYFDEKFKNFTTYFGGFHENRKNMYSDQAQSTAIAYRLIHENLPKFL |
| | | | | | ENIKIFEKIKAVPEVYEKCQVLYKEIEEYLNINSIDEAFELDYYNEVLTQKQIDVYNLIIGGRIPSEG |
| | | | | | KTKIQGLNEYINLYNQRQEKNKRIPKLKILYKQILSDRVSTSFTLDKFTKDQEVLNAVEEYYKFE |
| | | | | | LLDYKPKDKEDTENVLESVKEIVAHLTDYDLRKIYLRNDTKITTISQKLFGNYGVFSTALAYYYT |
| | | | | | TVIKPNFIEDHQKGNDKKREKLDNEQAQFLKQSYLSLDLLQIALDHYIPTLDQEEDVVKRYSSNC |
| | | | | | LANYFKDYFKAEKEEGKDKEFGLIDNIKAKYSCIQGMLGTDYPEDKKLVQQKEELFNIKAFLDS |
| | | | | | LMELLHFVKPLSLPNDSLLEKDQNFYGQFEPLMEQLNGLIPLYNKVRNYATQKPYSEEKVKLNF |
| | | | | | KNPELLGGWPVDREIATSSVLFRDGENYYLGILDKESKKDFKNLPKPDNPNDVLYKMLYLQAAD |
| | | | | | PSKDVQNLMVIDGKTVKKNGRKEKAGEFMGQNLILEDLKNTYLPDDINTIRKRKSYSKLSDNES |
| | | | | | KSDLIAFIDYYKQRAIEYFHNYQFQFKNSDEYRDFGEFTTHINEQAYQINFTEISWKFIDELVNEG |
| | | | | | KLYLFQIYNKDFSSYSKGKLNMHTMYWKALFEEENLKDVVYKLNGQAEVFYRKSSIKNPVVHK |
| | | | | | ANEAVANKNPLAQKKESVFEYDLIKDKRYTEDKFHFHVPITLNFKATGKEFINQDVIEFLHQNPD |
| | | | | | VNIIGLDRGERHLIYLTLINQKGEIILQESLNNITAENHPITTPYHELLNKKEKERDEARKSWGTIE |
| | | | | | NIKELKEGYISQVVHKIAKLMVQYNAIVVMEDLNFGFKRGRFKVEKQVYQKLEKMLIDKLNYL |
| | | | | | VFKDVEPNEPGGLYNALQLTNKFESFQKMGKQSGFLFYVPAWNTSKIDPTTGFVNLFDTRYENL |
| | | | | | EKAKAFFSKFEEICFNPIDKYFEFKVENYASFNAKAEGSQQDWTVCSFGERIKTFRNPHANNQW |
| | | | | | DNKVNQLTEEWQGLFKKYNIDYRTELKEQILQQTEAGFFKAALDLFKFTLQMRNSITNSEVDYL |
| | | | | | ISPVKNKQGQFFDSRQADATQPKDADANGAYHIAKKGLMWLAQINAFDGADWKKLDLDKSNK |
| | | | | | GWLQFIQNKK |
| MG29 | 6430 | MG29-107 effector | Protein | Unknown | MGMIGDQFIGQYSLQKTLRFELRPIGETQKLLQDFKEEVQGNLLEYDAERARAYPMVKKVLDD |
| effector | | | | | YYRYFIDQVLSGFAFDSQTINEVYEMYKKAKKDAEAAKEYAVHTKKLREQLSAAFKAPITYYML |
| | | | | | DKYEHLFNRNRESRLFEWLDIRFENDHLTENEYDEIKDVLDKFDKFTTYFTGYKENRANLFVAD |
| | | | | | EKATAIAYRVVNENMPRFFENCIRMENIKKRHLDLYKLLDSFEGYFVPQAYANIICQPAITDYNKI |
| | | | | | IGRPTQNPDEKGVNSIINEYRQKNQIKNRELPMMAQLYKQLLSDRITVFLDPVINNDEEMQSIVA |
| | | | | | ETIEIARGLFSEVINLTAIHALADNSENIYINSSALANLSHRVYDDWNLIYRACEAKMIKLEGKQK |
| | | | | | KGLENKLKMAIPMSELQNIJEEYIATLDEELKLSYYKIPILCNYFQNPPLDDFESATLKFEQIVKTT |
| | | | | | TPRTDLIHAIKEVLDKAMEVVRFFKPLYLFKGRSPLEVPDRNEDFYNEFERLYAELNLISKIYDRV |
| | | | | | RNYATKKQFSQDKIKLNFNNPTLLDGWDLNKEQDNLCVILIRDGNYYLALMNRDYRRLFDLKN |
| | | | | | DEVRNKALGKAGDHCYSKLEYKQVTGANKMLPKVLFAATNSDLFKPSQEILDIRKTGSHKKEA |
| | | | | | GNIEALHKWIDFCKQSIATHPEWNDHFDFKFRSTSEYSELTEFYNDFDRQAYKIKFVDIKVEYIDQ |
| | | | | | LVKEGKLYLFQIYNKDFSPYSKGRPNLHTTYWRMLFGNENLANITMDTDRPIFKLNGEAEIFFR |
| | | | | | KASLEKQITHAKGQPITNKSKKDNGKESESIFEYDLIKDKRYTEDKLFFHCPITINFRAPGTTVGS |
| | | | | | FNRKVNYFVERNPEVKIIGIDRGERHLLYYTVIDQKGNILEQGSLNQIHNSYTSAGRVVEHNINYR |
| | | | | | DLLHEKEKGREEARKNWETIENIKELKAGYLSQIVNLLSNLMIKYNAVLVLEDLNAGFKRSRIK |
| | | | | | VEKQVYQKFEKAMIDKLNYLVFKELPPGSSGHYLNGYQLTAPFTSFRDLGRQSGFLYYVYPSYT |
| | | | | | SHICPKTGFVNLLNTRYESIEKAISFFEKFNSIKYNPGSDYFEFDFDYASFGKDVARSQWCVCTAG |
| | | | | | EKRYYYANHDKTSRECNATQQIKELLDKYNIEYIRGKDLLPEIIKKNDKGFFNGLMFLLGVVLQ |
| | | | | | MRYTVSGTSNDDDFILSPVMDEQGQFFDSRSAATSEPQNADANGAYHIALKGLKIISSISDGKLKT |
| | | | | | VNKNERQDWFAYVQNKMYR |
| MG29 | 6431 | MG29-108 effector | Protein | Unknown | MEQKKSIWDECTNLYSLQKTLRFELVPVGRTREHINQKRLIEEDEELAKKENDAKKIMDDYYKF |
| effector | | | | | FIEDRLKKVRIEEANLKEFEKIYRDLKKDRSKDKKKRDEFSKIQKKIRNDIHKKLFEDTPEKSFG |
| | | | | | KDFLTKTLPEWLKSNNREKDSSTIKDFQRWTTYFKGFFKNRENVFSKDDIHTSFIYRIVHDNLPK |
| | | | | | YIDNLDRYDTLKRYSDFKDEQLCKSFSSELKGQTLSEFFIVENFNECLNQSGIERFNLIIGGKSLDD |
| | | | | | NKKIQGINETINLYSQRKTYEEKREVRRLFMLPLFKQILSDRTSSSFVLSAIKDDAEVISYINDFYSI |
| | | | | | ISNYFELLQTVFKQLFEFNTKQIYIHKASLNDVSRQIYDDWMILETGLKFFAKEKLKLTTEKKIEE |
| | | | | | WFKKKAYFSLDEIKQSIEYLQSENVDPKKVVEYHKTITRDNKPIIDAIKADYVAVKELNLNKGER |
| | | | | | KLLTEQREEDVTKIKQFLDSVMNLYHFLKPFSVGVDADSETGSFAVEVDAEFYKDFSEIYARLAE |
| | | | | | VVPLYNKVRNYITQKPFSRNKFKLNFNESALLDGWDKNKEHDKNYSFIFRENDSFFLGITSTDED |
| | | | | | TNILREDIHPEIFVEDSFFRKMVCKDLGDIKRQLPRMGFSNKAKTGVEVIGWNQDIEDIKKDFDE |
| | | | | | FQEKKKNDKKLWGENFDRNKLVKLIDYYKSVLKNHSEKYEETNNITYKPTADYRNLGDFFDHV |
| | | | | | QSQTYKVTFVGIDKDYIDALASEGKLYLFQIWNKDFSPLSKGRKNLHTLYWQELFSPKNLSDVR |
| | | | | | FKLNGKAEVFFRRKSIAPIITHPKNKPITNKNPIRGKKERTLSYDLIKDRRYTEDKFFFHCPITLNF |
| | | | | | KSSGSDNNKYINLKINDIIKKNPDVNILSIDRGERHLLYYTLLNSKGEIREQKSLNTVFDDVQREHS |
| | | | | | YHDKLNTLEVERQAARKAWKTIRNIKELKSGYLSQVVHKICKLSIDHNALIVLEDLNSGFKRERF |
| | | | | | KVEKQVYQKFEKTLIDKMNFLVFKERPHNIPGGVLYAYQLTSKFDTFKKMRKQTGLLYYVDAR |
| | | | | | YTSKICPKTGFVNLLYPKYTNVSAAQNFFRNFESITYNTKEKYFEFKFRYSKFISEQKPSARKNSD |
| | | | | | LPNNYYQKEWTVCSFGNRLVPKRSSKNIVEILDLNPTQ |
| MG29 | 6432 | MG29-109 effector | Protein | Unknown | MKLTDFTNLYSLSKTLRFELIPQGKTLQHIHEKRLIEQDKQRDIDYKKLKKIIDEYHKQFIDQSL |
| effector | | | | | KGIVLNDLDSYRTLYQKKEKDDQEKKNFDKVKSLLRKQISDAFKSQEKFKTLFAKELIKDDLLK |
| | | | | | FVQEEEKELVNEFKDFTTYFTGFHENRKNMYVADEKATSIAFRLINDNLPKFIDNLNIFEQIQKEN |
| | | | | | SNIIENFRTALLEMEDMTQGLELEELFSLNYFNETLSQRGIELYNTMVGGRFVEEGKDKIKGLNE |
| | | | | | YVNLYNQQQSDRKKRLPKFKQLYKQILSDRNSTSFVIDQFQNDSELLECIEQFYQSGICNYESDG |
| | | | | | NTINVLLSLQNSLRSLITFDLSRIYLRNDRSITDISQSVFGDWNVIRSALSDYYESTFPINAKEKIAEY |
| | | | | | EDRKEKWLSKSGYFNISTIQTALNGYKNDNITIKDRGNELINYLASCGFDLESNLNLVEKISKSYA |
| | | | | | DIRDLLNQPYPDQENLKNDKKNIGLIKQFLDGLMDFIHFIKPLNVNEEGLEKDQTFYSLFEPLFD |
| | | | | | QLSKTISLYNKVRNYLTQKPYSIEKIKLNFENKGQFLGGWVDSKTEDSDNGTQAGGYLFRKRNE |
| | | | | | IGEYDYYLGMSNDVKLLRTYLKHRIQEKDKSDYERMDYYQLKTASIYGNSYMGNLSYKEDKER |
| | | | | | LISAIVNYLKKNGEETAFLAINDYLNNNKLTESRTPSGCLNILSKDYQHTFKRIIKDESFLKVNNEV |
| | | | | | VQNLKETLKTLVRVPKGMEYANKQYTLFPEVIADIEELSKIKLFLYFPVCQKELDDALIRTHKPL |
| | | | | | FLFKIVNKDLSFAEEFIKGKRKSRGKDNMHTLYFKQLMSGAQDIIDIGTGEVFYREASIEGNITHP |
| | | | | | AHQAINSKNPLKKGEKRTFKYDIIKDKRFATDKYFLHLSITLNYQKPKQAKDYELIVKKYLRNNP |
| | | | | | DIKIIGIDRGERHLIYLTLIDQQGNIIKQESLNIIEDKKHNINTPYHTLLSEKEKGRADARVSWNTIE |
| | | | | | SIKELKEGYLSQAVHKMATMMVKHNAIVVMEDLNFGFKRGRFKVEKQIYQKLEKMLIDKLNY |
| | | | | | LVFKDIEPTAPGGLSNALQLANKFKSFEKINEQSGFLFYVPAWNTSKIDPATGFVDFLKPKYENIE |
| | | | | | KAKDFFNRFQKIRFNPQKNYFEFHFDYNDFTTKAEGTKTEWTVCTFGDERYAWNRKLNLGKGA |
| | | | | | MEKINVTESLQLLFHDFDISYATGNNLIQDILEQNNADFYKKFIRLLSISLNLRHSNGKTGNEQKD |
| | | | | | FILSPVCNNQGEFFNSENAGETMPKDADANGAYHIALKGLWALNQIDKADDLKKIKLAISNKEW |
| | | | | | LQFVQTKPYNKK |
| MG29 | 6433 | MG29-110 effector | Protein | Unknown | MQTVFDQFTGLYSLSKTLRFELKPIGQTKELLEDFYKHCEGNPIVVDEQRICQYPKMKDLLDDY |
| effector | | | | | YRLFIDKTLSRPIFSAEEITKAYELYCAAKQSHNSKQKESKYQKEYTAAKKSLRKKLAACFAEQK |
| | | | | | EEFGLDKYSHLFGSEQLPLNCWLQSRLQGGQITAAEYEEGLAALKAFERFTTYFTGFKENRDNL |
| | | | | | FAEVDKASAIANRVIEENMEKHFFNCRALADSTQKYPQLAEELGSFLSFFMPAYYGSCLSQQGID |
| | | | | | CYNQAIGKELNGDSTKGVNQIINEYRQKYSLKTKDLPTLITLHKQLLSKKPDCPVSETLTTDQEM |
| | | | | | LQLAKYCYTTAISRLNELQKIMSDYLNDENLQFIYLKTKDLNALSKKMFGEWDTIRNAYFYHCQ |
| | | | | | QLADKEQKRFAANTKEVISLGLLQRLLNSYLPGSENPPPLVDPALYFKSFDLTPLRQAYEAAAPV |
| | | | | | LALTQLDQDKAPPNEGNPKGGLGYRQTILVKNLLDTILRAKDFYKPFLLEQDGKPIAVADSNELF |
| | | | | | YTQFTAAFAKLNCLYKKYNLIRSYASQKPFSTDKFKLNENNSTLLSGWDLNKEEENTNILLRKDG |
| | | | | | QYYLGILKNTKLFADCSKYLCQDSVEHYEKMVYKQVSGVNKMFPKVFFAAKNLELYKPSAQIL |
| | | | | | QIKAQKSHLKEANNPDAKNAWIDFCKDSIAKSEWPQYFKFKFKPAEQYPDVNSFYREADAQMYS |
| | | | | | LTFQNVAGAFVKQAVENGELYLFEIYNKDFSAYSKGKPNLHTLYWQMLFDEQNLHNIADNAEQ |
| | | | | | PVFKLNGEAEIFYRKASLDDRVTHPAKKPICNKNPLNGKKTSTF |
| MG29 | 6434 | MG29-111 effector | Protein | Unknown | MKSFSNLYSLSKTLKFGLIPEGDTLSNIEKAGILAEDEKLAEDFKKVKKIADQWLKTFINDSLTGA |
| effector | | | | | YLNLEDLLIFEEKYSIFPRDEKDEEEFNDIKAKLRKEIVSYFIRNPKFKLIASADLIRKELPAFVVTD |
| | | | | | EEKNLVAKFKTFTTYFTNYHKTRENIYSAEEKHSSYAYRVINENLPLFIANKKNFETIKNSYPELIE |
| | | | | | DIKKSVEPILNGEKVEDMFSLKWFSKTLTQSGIDLYNKMIGGESLEDGKKIQGFNEKVNLFRQSN |
| | | | | | KLDGKSVPMLKQLRKQILGDKNVPSWVTEGFANKESMKNAVMEFHGKIKPVLFTVADIFAIEN |
| | | | | | CDYSNIFIKSRFLTDLSHELFKDFNFLKNILLEKYIVNNPKSKNQEKEFLGIAYFTVADIQSVLPNT |
| | | | | | DKDFIFEFFYSKIINTVAGIRQSYELWTANQESVPVLKSLMDNIIALHRTLKVFDVEGLGKDPVFY |
| | | | | | EPFDRNFEGMDGAVKFYNEVRNFITKKPYSLEKIKLNFGNSTLLAGWDANKELDNSSVLLRKGD |
| | | | | | YYYLGIMDKKHNKIFRNAPLAEKGEESYRKMEYKLLPNSYMMLPKVFFSKGNMSKYEPSEEILK |
| | | | | | IYETGAFKSGDNFNIDDLHLLIDFYKESIAKNEEWVCYNFNFKPTEDYQKINEFYNDIDSQGYNVN |
| | | | | | FRDIKASYIDELVKEGNLYLFKIYNKDFSPYSKGTPNLHTLYFKMLFDERNLKNTIYKLNGGAEIF |
| | | | | | FRKKSLNYSEETLKKGHHAEELKDKFDYPIIKDRRFAFDKFQFNVPITLNPNALGFGNINDAARD |
| | | | | | FIRSNDIKVIGIHRAESHLVYLVVLDSKGKIIERQSLNEIEGYNGKSINYMEKLDERGKERDEARV |
| | | | | | NWQEIGNIKELKEGYLSNIISKIAALMVRHNAVCVMEDLNYNEMRERSAIEKQIYQKFEKMLIDK |
| | | | | | LNFYVDKKKNPEELGGLLKPLQLANKFISFEKMGKESGMIFYVSPY |
| MG29 | 6435 | MG29-112 effector | Protein | Unknown | MKKTLSDFTNIYSLSKTLRFELIPQGKTLENIEIKGLLKQDEDRAEKYKKVKIIIDEYHKDFIEKSL |
| effector | | | | | NGLILDGLHDYMSLYLMTIKEDKDKKAFDKEKEKLRKQIANAFKNNEKFKTLFAKELIKEDLM |
| | | | | | NFANEQDYEYIKAFKDFTTYFTGFHQNRENMYVVDEKATAIAYRLINENLPKFIDNLKIFEKIKN |
| | | | | | EAPGLINQLNKVLSEMEEIVQGKTLEEIFSLNYFNQTLTQTGIDLYNIVIGGRTPEENKIKIKGLNE |
| | | | | | YINTDFNQKQTDKKKKQPKFKQLYKQILSDRHSVSFMPESFENDNQLLESIEKFYTNELLHYSTE |
| | | | | | GKSINIFEAIKNAVGNLSSFNLSKIYLRSDTSLTDISQKVFGDWGMINKALQDYYEKIYPLKPKEK |
| | | | | | QEKYEERKNKWLKQDIDIQTLQTAIDHYENETVKEKNNSKIITDYFANFGINNESKIDLLQNVYQ |
| | | | | | NYNIIKDLLNTPFPESEKLGSNKELVSLIKTFLDSIMNVIHFVKPLSLKDSDKEKDESFYSLFAGLY |
| | | | | | DQLNHTISIYNQVRNYLTQKPYSTEKIKLNFENSTLMDGWDLNKEADNTTIILRKDNLFYIGIMD |
| | | | | | KKNNHVFQHIPERTDNEPHYDKMIYKYFPDASKMIPKCSTQLKTVVSHFESNITDKIIEGKSFDSA |
| | | | | | LKITKRIFELNNFVYDDISKTMVLSEDNEKRPKMFQKKYLEISKDIDGFKDALKDWINFCIDFLN |
| | | | | | KYESTKHYSFNFKNSELYNSLDEFYGDIDTQTYKITYKNIPVSFIESLVNEGKLYLFQIYNKDFSPF |
| | | | | | SKGKPNLHTLYWKMLFDDENLNDVVYKLNGQAEVFYRKSSIKESNKIIHKANEVLTNKNP |
| MG29 | 6436 | MG29-113 effector | Protein | Unknown | ELEEAADALKPLLSLEQLNKKRIPPEGDSEEGSEGFEQVRRIHECLDAHMSLQQVLRPLHLVLG |
| effector | | | | | RKPIDVASKDLGFYARFDEAIEDYNAMTIALYNKTRNHLTKKSFSTDKVKINFESPTLLAGWDLN |
| | | | | | KETANKSIILRQNGKYYLGIMHPRHPKIFSKPPEAKVGDEAYEKVNYKLLTGANKMLPKVFFSK |
| | | | | | KGLETHNPSEQILALYKNGEHKKGDTFNIESCHKLIDFFKSRIPLYKRDPSDPYGWEIFDFKFSPT |
| | | | | | KTYKDLSGFYREVEEQGYKLWFTHVTKAYIDEQIEQGHLFLFEIYNKDFSPFSKGKPNLHTLYW |
| | | | | | KGLFEQNNLDDVVLKLNGEAEIFYRKIISIAANEQIVIISANKAIVNKNENNPKPESTFEYDLVKDR |
| | | | | | RYTKDKFFFHVPITLNHKAQKPVRENDQVNRALQKADDVHVIGIDRGERHLLYYTVVNQKGEII |
| | | | | | EQDTLNTISTDQDYVVDYHHKLDQQERTRDKARKAWTNIDNIKELKAGYLSHVVHKLAELIVK |
| | | | | | HNAVVCLEDLNFGFKRGRFKVEKQVYQKFERALIEKLNYLVFKDTTEGQPGHYLKAYQLTAPF |
| | | | | | ESFKLLGKQSGILFYVGASYTSKIDPATGFINFLKPHYESLAKSKTFFESMDSICFNAKRGYFEFSF |
| | | | | | DYANFSVPQTLDDYQTAWTVCTHGETRYHNQRNDKGIWETKAVNVTEKLKVLLNEAGVSYQN |
| | | | | | GEELKDAIAAVKSSKFYRSLYFLLRLTLSLRHSVTGTEEDFILSPVADEGGNFYDSRNASDAEPKD |
| | | | | | ADANGAYHIALKGLWNLEKIDQWDGESRLNLAMKNVEWFQFASEKPFKE |
| MG29 | 6437 | MG29-114 effector | Protein | Unknown | MMSNLLADFTNQYQLSKTLRFELKPVGKTAEWIEKHDIIGVKNDELFGKDAEKAKHYKYAKRL |
| effector | | | | | LDAMHRLFIEDALALFRNPEFSQPLESKFKVLQTQDELKIDDDLENIFKSVFDHTAIRWIEEYQQ |
| | | | | | QMPDFWQEDLAELEQKLATANDTKKRKGFQSAVKAIQKKLKDPSKVIKKEDIKVMYSNEDAIH |
| | | | | | LLEWKIRTEKVKITFKELEQGDNNNFIPVQILCEYLREFNQFYTYFSGFNENRQNVYDLSGEKST |
| | | | | | SIINRTINENLVFHFANLKKWQKVKKSLLDATNILAQKGFDAKASLAKIEADLQFNADAFFTVRT |
| | | | | | FCSSMCQSGIDRYNEIIGGQPALEGREKVQGINEFINLCRQQAGAKRQKFPPMQILYKQILSKSD |
| | | | | | KTFIPEFSDDKDLFNEIEKFHRDFFVEKNEKGRNLFENFIQASSELANDLTEEYDNIFLPCDKVNR |
| | | | | | LSNLLTGSWKNLKDEILEIIGEKEYTKRKNFSFTEITKALEEGKKMEHFSIDAKYSGQSLIHFFVT |
| | | | | | RFQEMLDTANTAWEDLLKKDVLNQEKLDDDRSEPGKKGFEQIAAIKAFLDAAIALAGFVRDWQ |
| | | | | | ADKDILKKDEPNRIWYDHLDDFTAKLQIIGLYNMTRNYVTKKPGATEKLKITFENATLLNGWDR |
| | | | | | NKETDNYGILLEKDHQFYLAVMTAKSNRLFDYAESSDDTEKKAQEKCEKRQLILASSNENCFRK |
| | | | | | INYKLLPGANKMLPKVFFAAGNEKLFNPSAEIVKIKQDKLYTKEKIEIHGKKNLYDYIDFCIKSLC |
| | | | | | KHPDWSKAFHFTPDSFKKPSEYESIDQFYRDVELQGYSISFDKIKESYINKKIESGELYLFQIYNKD |
| | | | | | FSQNKKKKGTDNLHTIYWKGLFEPENIKDTVLKLNGQAEIFFRKASIKYTPDKLAEGHHAKELK |
| | | | | | GKFSYPIIKDKRFTENKFFFHCPITLNFGAPSIPKKFDIKIRKFLKNNPKVNIIGIDRGEKHLLYYSI |
| | | | | | VNQDGKIMEQGSLNTISNGFIPNGTTESRPIDYHEKLDAVEKKRDIARKSWSMIENIKELKAGYL |
| | | | | | SQVVHKLAELIIKYNAIVVLEDLNMGFKRGRFGVEKQVYQKFEKALIDKLNYLVFKSEKDRSNA |
| | | | | | GHYLNAYQLTNKFESFQKMSQQTGILFYTTASYTSTTDPKTGFLKNIYNQYKSVEKSVEFWKSF |
| | | | | | DSIIYNQPKDRFEFTYTLGKVASKNMYREKDEKETQLKKRTWTVCSCVTRSRYKKAQESQTEE |
| | | | | | QKQNTSSEQIGKRGKHEIFFVTDKIKETLQNNGIDFTKNKDIQALLITKNARSDASFQYSMLYCF |
| | | | | | NAIMSMRVTDDDKEKGSKENDFILSPVEPFFDSRTAPSSFPKNGDANGAYNIARKGICILQKINAA |
| | | | | | DDVSKVSPGISKQDWQNYAQSH |
| MG29 | 6438 | MG29-115 effector | Protein | Unknown | IAYRIVHENLPKFIDNISIYENIKSNNKDLDFSPILNEMEDIIQGKTLDEIFTLDFFNNILSQNGIDFIN |
| effector | | | | | HIIGGRSGEAGEKKSKGLNEHINLYNQQQKDKKKRAPKFKKLYKQILSDRGSISWLPEAFEKDE |
| | | | | | EVLDAINNFYREGLENSVIDEKNVNILNEIESSFKSLLNYDDFSKIYIRNDTAITDISQTLFSDYSILG |
| | | | | | RALNYYYETFVNPKWITDYSKATETKREKLEKERDKFTKSTYISIDILQKSLAEYIKTLDTDSEIK |
| | | | | | QKYTPTLIANYFTHHFYAKDENGNETEKTLTYQIVSEYNGLKGFLNTEHSEDYKLIQDKERVHQ |
| | | | | | LKTFLDSIMNLLHFAKPLYLDKNASEEKNELFYTEFTPIYDELAKIVPLYNMVRNYLTKKPYSTE |
| | | | | | KFKLNFENSTLLDGWDVNKEKDNTGVILLKDDNYYLAIMNKQNNTVFEEIPKAINPQNTFKKMN |
| | | | | | YKLLPGPNKMLPKVFFSKSRTKEFGVSEKMLENYENGTHKKGDNFNLSDCHQLIDFFKASIQKH |
| | | | | | EDWKQFDFNFSETKMYEDISGFYREVEHQGYKITFSDVSEEYINQLVNEGKLYLFKIHNKDFSEY |
| | | | | | SKGKPNLHTLYWKTLFAPENLADVIYKLNGEAEIFYRKKSINIDKAVTHNAKEKLENKNPNATK |
| | | | | | KISVFDYDIIKDRRFTFNKFQFHVPVTMNFKSTGNDFINPHVNEFLKNNPDVKIIGLDRGERHLIY |
| | | | | | LTLIDQQGNIIRQESLNTIKDEKHNIETPYHLLLDNKEEERDKARKSWDTIENIKELKEGYISQVV |
| | | | | | HKIATMMIEHNAIVVMEDLNFGFKQGRFKVEKQVYQKLEKMLIDKLNYLVFKDKPANEAGGIY |
| | | | | | KALQLTNKFTSFRDMGKQTGFLFYVPAWNTSKIDPTTGFVDFLKPKYESIEQVKTFLRKFKNIYF |
| | | | | | NSTKNHFEFSFDYKDFTTKADDTQTEWTICTHGDRIEKFLNSINKWDERTVTLNDEFVTLFDKY |
| | | | | | HIDYKDSKILKEKILQQTEKTFFERLIYLLRLTLQMRNSKTGTEIDYLISPVADKNGNFYDSRKVS |
| | | | | | QNLPKDADANGAYHIALKGLWVLQQINKTEDLKKVKLAISNKEWLQFVQK |
| MG29 | 6439 | MG29-116 effector | Protein | Unknown | VISLYNKVRNYITKKRTSIKKFKLNFRNSTLLDGWDLNKEKDNFSIIFRKSKNYYLGIMNKENNKI |
| effector | | | | | FDNIPKEYLISNNNSYEKMIYKLLTNPNRMLPKVFFSKKKIKYFNPSGEILDIINHSSHTKNGKVK |
| | | | | | EGYSKKDFNLNDLYKIIDFFKKSIEKHPDWKNFKFNFKPTKDYKDISEFYNDISSQGYKLSFINISN |
| | | | | | EYIENLVNNCKLYLFQIYNKDFSKYSKGKKNLHTIYWKELFSKENLEDVVYKLNGKAEIFFRKK |
| | | | | | AFDAHITHPKNIEIKNKEFRKEYFKLGLKPTSKFKYNIIKDKRYTYDKFLFHCPITINFKAPSNNFY |
| | | | | | LDYKINEIIRNKSNSVKILSIDRGERHLAYYTLLNSKGEIEKQESFNLVNDKFGRKLDYHNKLDKL |
| | | | | | EGNRDQARKNWNTIENIKELKEGYLSQIIHKIAKIVVEENAIIVFEDLNSGFKRGRFKIEKQIYQK |
| | | | | | FEKMLIDKLNYLIFKDNDKYSIGGTLKAYQLTKKFESFQKLGKRSGIIFYVNASYTSKICPVTGFT |
| | | | | | NHIYPKFENIKKAQELFKKFKFIRYNVKKNYFEFNYDIKNFNKKLKLNRMNWTLCSFGDRLVTF |
| | | | | | KDKNGFFNTKKINLSDKLKDLFDTYNINYINERNLIDDIISQKDKKFFKNLIFFLKTLLSLRNSYTE |
| | | | | | SEVKEIMHKNSENFKLNDYDYILSPVEKDGVFFDSRKANNLQPKDADANGAYNIGLKGLMIIDRI |
| | | | | | KNTQESSNVNLNIKKEDFLNFVIEKNK |
| MG29 | 6440 | MG29-117 effector | Protein | Unknown | GGKGFQQVQQIKAMLEAFNNILHSLKPLHLVKGRKPIAPNDVDTSFYTLFSDRFEKYSNQLIPLY |
| effector | | | | | NKTRNHLTKKSYQTEKFKLTFSNPTLLDGWDVNKESDNLGVLLRKDGLYYLAIMHSEHRKIFE |
| | | | | | QAQEAGVGEDCYEKVNYKLLPGPNKMLPKVFFSKKGIEQYAPPAEILRLYKNEEHKKGETFKL |
| | | | | | DSCHKLIDFFKANISHYKKDANDVGWAVFEFQFSPTKNYEDISAFYKEIERQGYKIWFTNIASKYI |
| | | | | | SDTVTAGKLFLFKIYNKDFSLHSKGAKNLHTLYWQGLFEQNNLKDVVLKLNGEAELFYRKRSIE |
| | | | | | TSNQIIHLANQPIKNKTKQAKQGSSKFKYDIIKDKRYTQDKFQFHVPITLNFKSANVKQFNHNINT |
| | | | | | AIQHDESVNIIGIDRGEHHLLYYSVIDQNGTIIEQDTLNTIDTGGKGYIVDYKDKLHSKEQERDNA |
| | | | | | RKSWGMIENIKELKAGYLSQVVHKLAKLIIERNAIVCLEDLNSGFKRGRFGIEKQVYQKFERALI |
| | | | | | EKLNYLVFKDTERSDEAGHYLNAYQLTAPFDSFKKLGKQSGILYYVPAAYTSKIDPATGFINFLN |
| | | | | | THYESVKKSQTFFKNMDKICYNAKENYFEFHFDYNKFEVNQDLTGYQTKWIACTHGGDNRYHY |
| | | | | | NVKQRTLDKINVTDKLESLLQQYKIQYKHGEDIKEVIGKIDKADFFKTLLWLLRLTLTLRHSDR |
| | | | | | NNDEDYILSPVKDKNGVFFDSREQEKLPREQQTLPIDSDANGAYHIALKGLWNLQQINDWDSTG |
| | | | | | KLNLAMSYNDWFKFVRQLKQ |
| MG29 | 6441 | MG29-118 effector | Protein | Unknown | MKLNKFTHQYPISKTLRFELKPVGETADYIEDFKSQYLKDIVIQDQQRAEDYESIKVIIDEYHRHY |
| effector | | | | | IEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSIIEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNDLLKSL |
| | | | | | GKFIKEMVKEDGLFKKLEDSIKLITDADLHRTFIKNGVEITKISQSIFGNYSILKSAIYHHAESVLY |
| | | | | | PDPISGKISEALKEKRIKYVNKQVVFSIAELETILSNYASQMTDDNPDKEIIAQCENSEHPIRTYFL |
| | | | | | NAIENVKNDKDIEFGKAIENVLPLISLENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEEGKLFLFQIY |
| | | | | | NKDFSPYSKGKPNLHTLYWKALFDTENLKDVVAKLNGQAEIFYRKHSIKKDERTIHRANKSLQN |
| | | | | | KNENNPKKTSLFEYDIIKDRRYTVDKFQFHVPITLNFKMEKMTQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDSRNAPKAQPMDADGNGAYHIA |
| MG29 | 6442 | MG29-119 effector | Protein | Unknown | MLCSECVGGLKMTKFNEQFIGQYSLSKTLRFELKPIGKTAEMIKANGFLEHDTKRADNYVLVK |
| effector | | | | | RFLDDYYRYYIEETLKGKELDKDLVEKAYSAYIGNNADECEKINKELRERVSKIFVDKDKYGLE |
| | | | | | KYKELFSVKQNRKTNRTEGILLSWVRNNDKYSQADKEKYEELIVYFNKFTTYFKGFKQNRDNM |
| | | | | | FSAEAEVTAIANRIVNENMYRFFDNVKNFENIKEHYPDLYKEFVQVEQYFVPSAFGKILSQSAIDN |
| | | | | | YNQGVIGRFENDLDAEGVNQVINEYRQNCLKDGKEVKKLPTMITLYKQILSEDKSENILEKENID |
| | | | | | TDEKAINLVKESYKILTENVKEIKKLIDYSITEETLDTIYIKKIEISTVSNAIFGNREIIKDALNYKKE |
| | | | | | KYKEVVCVKTISDAIKEYKQVYDGEIFDWKDKNIDDLFVKYFSSFYTGYVTKSKNTKKQKDEIV |
| | | | | | NIIEYVDNIKKELDTIVSNDVRDYKNILDKMNAIVRFYKMFYLYDGMKKLDIRGKNEYFYNHFEL |
| | | | | | MYFDIREINKKYNEIRNYATKITQPNNGEKLKFTFDASTLLDGWDKNKEKDNLSILLVKNGKYY |
| | | | | | LGVMDNENKKCFDFENEDVAKKAGQVGETYQKMEYKQISFNYGGLGGFVRKCFNTAQQFRW |
| | | | | | TCPKDCLNEEGKIIITDEEARNNLVKIIDCYKDFLNKYEKNGFKYKDFSFQMLPSDQYSKLSEFTQ |
| | | | | | DIERQRYKLWFRNVSAKYIDELVEQGKLYLFQIYNKDFSDNKKQKGTDNLHTLYWKALFSPEN |
| | | | | | MNKTDGAIIKLNGEAEIFYRFKADGIPVIHKKGSILLNKISKYGETIPSKIYKRIFDCLNGRIEKQD |
| | | | | | LNEEERKWYDKAVWKEAKYDIVKDKRYYGDDGKYFFHCPITINFRCKEKVNGNTFNQEINQFV |
| | | | | | ANNPDIHIIGIDRGERHLLYYTVIDQQGNIIEQDSLNNISSDYVANNKLVPHKVNYHDLLDKREQE |
| | | | | | RADARLAWTAIENIKEIKSGYLSQVVHKLALLVEKYNAVIVLENLNVGFKRGRFKVEKQVYQNF |
| | | | | | EKALIQKFNYLVFKNRSYAENGSFANGYQLTAPFTSFKDIYRQTGILYYVDSSYTSHICPKTGFVN |
| | | | | | YINSYLKYTNVEIAKLTLAKFDGFKFNNAKGYFEFAIDYDKFINGKIPLGKWTICTVGQERYSYD |
| | | | | | AKNQTTIKYDVTKELQNLLVKYNVAYEDGKDLLGKIKDIDEKGFYSALLYWLRLTMQLRYTVK |
| | | | | | GTENEDDYILSPVADKNGNFFDSRKAQDNEPKNADANGAYHIALKGLQLIQNIDDGKLAKPEKN |
| | | | | | MENAKWFKFARERNS |
| MG29 | 6443 | MG29-120 effector | Protein | Unknown | MADNGRSLANFTNQYQLSKTLRFELKPVGQTAEWIKKHNIIDVDGDKLTGEDADRAVNYKYAK |
| effector | | | | | LLLDELHRQFIEDALKLAPEAEFTKKLKDKIIELYSASEIKDADLPGNIFKQILNNKADEWIKLYQ |
| | | | | | QEMPQYWQKEFDAKIANETSEKKIKYLRNSFDKLNKRCQEQPFKNSGVDVLYGSNEDPIKLLE |
| | | | | | WAVRKEKVRPTFGDIKHEGNPEAIMPKERIIEYIIRGFDNFCTYFAGFNENRANVYDVTGAKSTS |
| | | | | | LVHRIFMQNMQFHFNNIRKWEKIKASLEKYAKDLSEKNYNCHARLAECERELNFSANEIFSPEAF |
| | | | | | INFINQSGIDRYNEILGGLAQEGGKTKPRGINEFINLVRQQTGAKRNEFPPLQLFYKQILSKSDRT |
| | | | | | FISAFETDEEMFDRIKDFRQKCFIESEAGKLPVIQEFIKDITRLIAESLDEKTNIFISKDKLTRISQEL |
| | | | | | TGSYNTINLRMLSELGEKAFNQNGSFSIQQIDNALNAVVDGKKFSSQDQNIKAEYQSNSGNILFDF |
| | | | | | FSKRLDALFVSIEASWKILHESRVLTGKILDKNRENEGDKGFEQVAAIKGFLDNSIEILGFAKDW |
| | | | | | MLQEKKLSENINTVWYETLQLFCDQFPIIKLYNMVRNYVTQKAYSDKKLKLNFDNSTLLDGWD |
| | | | | | RNKESSNYGVLLEKDGLYFLGIMTPESNNIFDYEIADADSLNKKQEKQELANAIKSSGGEKLYRK |
| | | | | | VIYKQIADVSKDIFTLCWDENNNKAIRKTKGREAIWGENITRIKEAKSYQNNEADKKHYFGYLIK |
| | | | | | CANSYWKHFNIKLKPADEYEDFSSLIKDIDIQGYKINFDNIKKSYIDEKVAKGELYLFQIYSKDFSQ |
| | | | | | EKKGGGNDNLHTSYFKLLFDEENLKDTVLKLNGQAEIFFRKASVELTEEKKAKGHHYEELKDK |
| | | | | | FNYPIIKDRRFAEDKFFFHCPISLNFKAEKSIPQDKYKPSFNSNFNLQIKTFLQNNPEVNIIGIDRGE |
| | | | | | KHLLYYSVINRNGNVIEQGSLNTIAGFKGREIDYHANLDKKEANRDKARKSWSAIESIKELKAGY |
| | | | | | LSQVVHKLAQLIVEHNAIVVLEDLNYGFKRGRFKVEKQVYQKFEKALIDKLNYMVFKDKEHRL |
| | | | | | VSGHYLNAYQLSGSYHLDSLKSQKQSGILFYTAASYTSTTDPVTGFLKNVNVTYENVEESLKLWE |
| | | | | | SFDSIIYNPIKDRFEFIYTLGKIAGKSTDKEKDEEEISKKQWTVYSCVDRSRYIKPESTEEQKQASD |
| | | | | | RNSIGKLGKHEIFCVTNEMKKLFENANINYKEDKDIKKVLLEQNNASLHRSCLYFFNAIMAMRV |
| | | | | | TDSSKKSGTDENDFILSPVEPFFDSRKKYTKLPENGDANGAYNIARKGICMLNKIDAAENLPKINL |
| | | | | | LITKRDWQEYAQSDAVVKAQTAKLNK |
| MG29 | 6444 | MG29-121 effector | Protein | Unknown | MLQNFTNQYQLSKTLRFELRPVGKTKEHIEAKGLIIQDEQRAEEYKEMKKIIDRYHKAFIEDALN |
| effector | | | | | GIAIEGLETYEKLYFAIKDEKGKKEFEKLQDTLRKRIVELFKKHPKWSTLFKKELIRNELLTFLD |
| | | | | | SEEIPEEQKINEKEIVLKFVDFTTYFTGFHENRANMYIDEALHTAVAYRIVHENLPVFLGNKKTFE |
| | | | | | QIATKYPELIADSKDAIESHLFGAVFEDMFTLAYFSHTLAQNHIDLYNTMIGGKVSNDGSKIQGEN |
| | | | | | EKINLYRQKHGLSKRDLPNLKPLYKQILSDRESLSWLPEAFEDKNELAEAIKTFYQNNIIAFECCD |
| | | | | | GKVNLLEKFPEIFKENQYYDLSKIFIKNDKSLTDIAQAIFGKYGVIKEALWEKHLRDNPKAAKSK |
| | | | | | DISADEERFFNKKDTYFSINDIHIALKAGEDRVQTGLDFSINDIHIALKEAQSPSDILTYFSNEIKPL |
| | | | | | TKAVQVAYKTWLDDQEKTERIKEVMDALLAWQRFLKPLSVKSDVDRDIAFYATFESYFESLSAV |
| | | | | | VKLYDKVRNFMTKKPYSLEKFKLNFENSTLLDGWDVNKETDNTAILLEKNGLFYLGIMDKKHN |
| | | | | | RVFKNTPESQDDSSYRKINYKLLPGANKMLPKVFFSNSRIDEFAPSSDIISNYKKGTHKKGEIFDL |
| | | | | | EHCHKLIDFFKSSIQKHEDWKNFEFKFSDTASYKDLSGFYREVEQQGYKITYKNISQSYIDTLVSE |
| | | | | | GKLYLFQIYNKDFSPYSKGTPNMHTLYWRALFDEKNLADVVYKLNGQAEVFYRKKSIIYSDEVM |
| | | | | | QKGHHAKELVGKFDYPIIKDRRFAFDKFQFHVPITLNFKAQGYTNLNAIVNEMIASGKEDIKIIGI |
| | | | | | DRGERHLLYLSLIDAQGKIVEQYTLNQIINSYNGKDHVINYHEKLAKKEDERAKARVNWGTVEN |
| | | | | | IKELKEGYMSHVIHRIATLMVEHRAIVVLEDLNFGFKRGRFKVEKQVYQKFEKMLIDKLNYLVD |
| | | | | | KKKSPNELGGVLNALQLTNKFESFEKMGKQNGFLFYVPAWNTSKIDPVTGFVNLFDTRYASVEK |
| | | | | | SKEFFGKFKSIRYNSEKDYYEFEIDNYTQFNPKAEGTRQNWTICTYGDRVLTYRNSEKLNQWDN |
| | | | | | KTVQLTDGFKTLLNGQADNLKEYIVAQNDKAFFEKLLGLFRLTLQMRNSITGTDIDYLVSPVVD |
| | | | | | ENGVFYDSRVCDDTLPKDADANGAYNIARKGLMVVKKIKEAQDIKKPDLKITNKEWLQFAQR |
| MG29 | 6445 | MG29-122 effector | Protein | Unknown | GLKEYFIKNYNLSNKKIESRLNQKYFSISEIQEGVKLLNLDRINYNDFSDHFISDYFKNLINEKIIDE |
| effector | | | | | ITNHKLDFDKINYNNLNSFSDNEKQLIKILLDSILGFYNSIKPLYVNIKSSQEEKTQEAYELDSDFY |
| | | | | | NDFQIIIDSFKKIIPIYNKTRNYLTKKPYTTKKFKLNFDNSTLLDGWDINKEKDNYSLLFKKDNQY |
| | | | | | YLGICSKGNSTDISKYIQKKVFNSGDYFEKIDYKLLPGPYKMLPKVFFSKTNIEYFSPSEEIISIRNY |
| | | | | | ASYSKNGTPQKDFDKEEFNITDCHKLINFYKFSLNKHHEWKNFNFNFKPTDQYKDINEFYQDVE |
| | | | | | DQGYNLSFKNIDSKYILDLVDSGKLFLFKIYNKDFSKFSKRTPNLHTIYWNELFSEENLSKLIYKL |
| | | | | | NGKAEIFFREKSNIKNNTIHGKNQLIQNKNPINNKTESIFEYDIIKDKRYTQDKFLFHCPITINFKSR |
| | | | | | GNGKDIHKQINNYIKDFEGNINILSIDRGERHLLYYTLLNSDGKIISQNSFNNISDGFNRSFDYQDK |
| | | | | | LDQREKERDQSRKSWTAIENIKYLKEGYLSRVIHEIAKIAIENNAIIVLEDLNFGFKRGRFKIEKQI |
| | | | | | YQKFEKMLIDKFNFLIFKKRSKESIGGALNGYQLINKFESFSKLGKQSGILFYVPASYTSKIDPTT |
| | | | | | GFFDLIRPKYESVDKSIQLIKKFEYIKYNSDMDMFEFNYNYFNFNNELKLDRKNWCIYSNGSRLY |
| | | | | | NFRNKDKNNEWDTKEINLTKELKDLFESYSIDYNSTQNLIDRIILIDHKDFFEKLIYILKLMLQLR |
| | | | | | NSIPNSKEDYILSCV |
| MG29 | 6446 | MG29-123 effector | Protein | Unknown | MGSIFDDFTNKYPVNKTLRFALKPIGDTEENIKKKGILLKDETRAKDYKQAKKIIDEYHKDYINR |
| effector | | | | | KLKDFSFDEGKLEDFADTYRNIKRDTQDENLKRQLEKLRNHLRKEVANHLKNKKLFSNQFIKDI |
| | | | | | LPQWLERNSISTEGIEKPEEVIGKFQQWTTYFTGENDNRKNIYTDKDHGTSIGYRLIHENLPKFVD |
| | | | | | NIERYDKAKSLNVDFSDVERSLVDKLPTGKLENFFTLENFNRCLTQKGIDNYNLIVGGRSENDGT |
| | | | | | KKQGINEIINLHAQQLKTGEAKKVRSCQLEQLYKQILSDRNTSSFRFENIETDSQLCGEIIDVVNTI |
| | | | | | DNVKENFTRVFYTLENADPKHIYIKSNKGITDISQNIFRDWGLVTRCLEHYVEAKIYPVSDGKKE |
| | | | | | TKTLQTRREKWLKQPFFSIDDIHTALESYFAQYKNEDLQNGTSAEGEIEIAEQKAIAINKPLFDYF |
| | | | | | KKCTIRKNNENDNRFESKNLFEEVQRTRKEALKVLEEFQDIEDQKLKNDKEKVQHIKNYLDALL |
| | | | | | GLQYFLKPLHFQLRKSEEKNANAYEKDGSFYTKFDGLYEVVQQIQPLYNKARNYLTKKPYSEEK |
| | | | | | YKLNFENQTLADGWDKSRETSNSCVLLLKNGRYYLAVMDKEHKRLFEGDIPEHKDCYKKMVC |
| | | | | | KLLPGPNKMLPKVFFSDRRGELFDPPEEILHIRNVASHVKNGDPQPGYIKQEFNLDHMHKMIDF |
| | | | | | FKTSIEKYEKWRNFGFQFSSTKSYENISSFYKEVSDQGYKIAFQNVSKDYIDECVKKGHLYLFEIY |
| | | | | | SKDFSDKSKGKPNLQTLYWKALFDEKNLRNVVYKLDGKAELFHRKASIHYKDEIWEKGHHADD |
| | | | | | PKKKQPYPIIKDRRYARDTYHFHVPITCNFKAENSGNFNEEVTRHLKNNDEVRIIGIDRGERHLA |
| | | | | | YYTVIDQRGKIIEQCSLNNLNGKDYHDILDKRENARDKARKSWGTIEKIKDLKEGYLSQVVNNIA |
| | | | | | KLMIEHNAIVVFEDLNFGFKRGRFKIEKQVYQNLEKRLIDKLNYLVFKDKKPHKNGGTFKALQL |
| | | | | | TARFESFQKLGKQTGFIFYVPAYHTSKICPATGFVNLLYPKYETIKKAQELFGNFDKICYNKDED |
| | | | | | YFEFHLDYGKFTDKAEGGRRKWVICTHGTRLENFRDENNQWDTREVDLVEEMKKLFQNNTIDF |
| | | | | | KNGECLIQKIKTKDGADFFRSLIKLLRLTLQMRNSRIGKEEDWLISPVKDNGVFFDSRKADPSMP |
| | | | | | QDADANGAYHIALKGLLMLKQLKGAGSVTDFKPDLSNKAWYEFVQEKPRL |
| MG29 | 6447 | MG29-124 effector | Protein | Unknown | MENSKSIWNSFTNEYSLSKTLRFELKPVGRTLELIKENKLIEEDKEREKEFNRVKEIMDEYYKYFI |
| effector | | | | | DICLSDIEISGLKKHVEIYYKLKKANYKDEKLKSEYKESQHKITKEIYDQINKDDALNKRLGEEFI |
| | | | | | RQILPKWLETNGRIEDKKLVLKFQKWTTYFINFFSNRKNVFSKDEIPTSIIYRIVHDNLPKFLDDIA |
| | | | | | KFEELKNLKGFDYTKIEKDFSNELNDKSLEMFFSVNNFKNCINQSGIERFNLIIGGKSESNNIKIKG |
| | | | | | LNEYINEFSQKEKDNEKKIRQLKLVPLFKQILSDRNSPSFILEQINDKKEMLDKIEEFYTSFNNNLN |
| | | | | | KLKNSIKNLKKFDSEKIYLKNDESLNSISKNMFGYWGKIQEGLKLYFESKLKNKKDIEKAQNSEY |
| | | | | | FSISDIDEGIKILQLESEYPVIEYFLKFSEDYQSSNSSDKNLLSQISSKYDILKDKIKEIRENDTNKISE |
| | | | | | DDVVKIKDFLDSILSLCRFLKPLYLYSDKDKSEELELLDIDPDFYNEFNPVFQELERIVPLYNQIRN |
| | | | | | YITKKSFSTKKFKLNFDNSTLAVGWDLNKEKSNYAVLLRKKNTVTEQYDYYLGIMIKNFNKIFD |
| | | | | | NGKKSTNDQNSYEKMIYKLIPGPSKMIPKVVFSKANKDSLNPSKEILKIYNESSFTKNGSAQGGY |
| | | | | | KKKNFDIENLHKMIDFYKFCLKNYEGWSTFDFRFKSTNEYSDISEFYKDIESQGYKISWKYYDEA |
| | | | | | YINEIVDNGKLYLFQIWNKDFSEYSKGKPNLHTIYWKSLFSEENLRDIIYKLNGGAELFYREKSIP |
| | | | | | RSITHEKNKPIKNKNPFDGKEESTFEYDLIKDKRYTEDKFFFHCPITLNFVKNYQKRMISKSVNKF |
| | | | | | IHDEKDGINVLGIDRGERNLAYYSLINSNREIIEQGSFNVIFDDKKRKLDYRDKLDEIEGKRDEAR |
| | | | | | KNWKNIANIKEMKEGYLSQVIHRISKLAIEHNAIITLEDLNFGFKRGRFRFEKQVYQKFEKILIDK |
| | | | | | LNYLIFKDTTEKEAGSILKAYQLTDKFESFQKLGKQSGIIFYVNASYTSKICPKTGFVNMLNPKFE |
| | | | | | NVNKSKEFFAKFKYIKYNRAEDLFEFNFNYSDFTDKNEIKLKKDNWSVWSNGIRLKTRKDNNGN |
| | | | | | YWKTEEIDVTAELKKLFKDNKIDYNSEINLTGSISNYNNQRFHKDLIDLLKLILQLRNSYTSYEVE |
| | | | | | EFKKKYGDKFNMRDYDYILSCVKDNKGEFFDSRKSKNNEIKDADANGAYHIALKGLMVIKKIKE |
| | | | | | SDNTDKTDLRINKNDFINYIIDSYGGKHNNI |
| MG29 | 6448 | MG29-125 effector | Protein | Unknown | NYVTQKPYETSKVKLNFENAQLLNGWDKNKEADNTSILLRKDGLYYLAIMNKKNNKVFEKAPK |
| effector | | | | | SIIGDVSFEKVNYKLLPGANKMLPRVFFSNTNIDFFNPSEEILRIRNTATHSKNGEPQKGFEKADF |
| | | | | | NLKDCHTLIDFFKDSLTIHKDWKHFGFQFSVTSTYQDISGFYREVENQGYKITFDNIPTSYINEKV |
| | | | | | EHGELFLFQIYNKDFSPFSKGTPNMHTLYWKMLFDEQNLKDVVYKLNGQAEVFFRKSSIQPKNII |
| | | | | | THPKNQPIDAKNPLKPNKNTFAYDLIKDRRYTIDKFQFHVPITMNFKANGTDRVNEKVNTFLKN |
| | | | | | NPDVNIIGLDRGERHLIYLTLVNQKGEIIKQESLNNIKDEKHNITTPYHTLLDNKEKERAEARQE |
| | | | | | WGTIEGIKTLKEGYISQVVHKIATMMLEHNAIVVMEDLNFGFKRGRFHVEKQVYQKLEKMLID |
| | | | | | KLNYLVVKSADKNKPGGVMNALQLTSKFESFQKMGKQTGFMYYVPAWNTSKIDPATGFVDFL |
| | | | | | KPKYESVSKAQDFIANFNAICFNKNKEQFEFSFDYNNFHNKAEGTQTLWTLCANNHTRYWYNRT |
| | | | | | LNLNKGGQEAIKVCQVLELLFADNNITYGNGADIRDQIMAQSSTDFFKKLLKCMSVTLSLRHNN |
| | | | | | GLKGDAEQDFILSPVSNDKGEFFNSLTAPATEPQDADANGAYHIALKGLWVLQQLSQTVDLSKP |
| | | | | | KLAISNKEWLNFVQKREFIK |
| MG29 | 6449 | MG29-126 effector | Protein | Unknown | SRFEKIKEFNLDLKTLENDFKDVLDNRDLNEFFSINNENNFLNQSGIDKFNLVIGGKSLEDNKKIK |
| effector | | | | | GLNEYINEFSQKESDKAKRKNIRKLKFAVLFKQILSDSESSSFVIEKFKDKKEIFETIDMFYEKFNK |
| | | | | | YSSKIKESIKKLNDCDSKNVYIKNDTNLTQVSKGLFNDWNKIDIGLRHHFENELKIKKLTDKQRE |
| | | | | | KELDKFMKSKYFSLDEIEKGINSLELKDKKSIIDYFLNFSKSKNDSKVDLFENIKSKYSEFNKINRN |
| | | | | | KTIKLTEKSSENDVELIKTFLDAIMELYHFIKPLLLNFKKNEDKKGSNVLETDSDFYNYFNEIFDK |
| | | | | | LGEIPLYNKVRNYVTQKPFSTKKFKLNFENSTLAAGWDINKETANTTIILKKGTDFYLGIIDKNN |
| | | | | | TKIFLNQQNSNSSVVYEKLCYKLVSGANKMLPKVFLSEKGVKTFKPSKEILNLYKNEEHKKGNT |
| | | | | | FSIESCHKLIDYFKECIPRYKPNPNDKYGWDVFKFKFSDTKTYKDISDFYREVENQGYKIWFENID |
| | | | | | ESYLNKLVDEGKLYLFQIWNKDFSKYSKGKPNLHTMYWKELFSEENLKDVIYKLNGEAELFYR |
| | | | | | EASIKRQITHPKNISIDNKNPIKNKEKSTFNYDLIKNKRYSEDSFMFHCPITLNFKAKDQSKSIHKL |
| | | | | | VNKFIHDTDKKINIVGIDRGERNLAYYTLVNSDGNIIEQESFNIISDDLQRKFDYQEKLDQIEGDRD |
| | | | | | KARKNWKKIANIKEMKTGYLSQVIHKISKLVIEHDAIIVLEDLNYGFKRGRFKIEKQIYQKFEKM |
| | | | | | LVDKLNYLVFKGIDKNLPGGNLNAYQLTNKFESFQKLGKQSGIIYYVDAYKTSKICPRTGFVNLL |
| | | | | | YPKFENIIKSQEFIKKFKSIKYHKEEDLFEFNFNYSDFKKDQKEKLEQDNWSIWSNGTKLINLRD |
| | | | | | KENNNQWTTKEYDVTEKLKELFNKYKIEYNSEDYLLNEIVKIKDKSFYESFIYILKVILQLRNSYS |
| | | | | | DFEVKQFKKKLGDKFKESDYDYILSCVKDKKGNFFDSRHAKTDEVKDADANGAFHIALKGLMV |
| | | | | | IDKIKKLNDVDEKTKIDLKIPRNDFLNYVVKRNT |
| MG29 | 6450 | MG29-127 effector | Protein | Unknown | QTVQEALSQCSIEAVKEKNKQNAIVNYFADMGLNEEIKMNLFDKVIADYKNVKDLLNTSYPENK |
| effector | | | | | KLGNQKGKDSDIEKIKTFLDSVMNVIHFIKPLNLKDESKEKDETFYSLFLPLFDQLNKTILLYNQV |
| | | | | | RNYVTKKPYSTEKIKLNFENSTLLDGWDVNKEQDNTSVILRKDGLYYLAIMDKSNKKIFLNAPK |
| | | | | | ADDKKDSFKKMNYKLLPLVNQQLPRVFFAKSRIEYFNPSSKIVQNYKNNTHKKGDTFNINDCHA |
| | | | | | LIDFFKASLEKHEDWKHFNFKFSPTKTYQDLSGFYREVEQQGYKMTFENIPTDYINEMIEEGKLY |
| | | | | | LFQIYNKDFSPYSKGKPNMHTIYWKMLFDEQNLKDVVFKLNGQAEVFFREKSIKDNIIVHKSNNS |
| | | | | | IENKNPDNPKKQSKFNYDIIKDKRYTIDKFQFHVPITLNFKATGRDYINEDVNRFLKNNKEVNIIGI |
| | | | | | DRGERHLAYYMIINQKGEILEQTSFNIISSKHKENKYSTNYHALLEKKEMARDKARKSWDTIGTI |
| | | | | | KELKEGYISQVVHKIAQLMVKHNAIVVLEYLNPGFKDSRKKVEKQVYQKLEKMLIDKLNYLVF |
| | | | | | KDYEANTTGGVLKALQLANKFTSFDRLGKQSGFLFYVQAALTSKIDPATGFVNFLYPKYESIKK |
| | | | | | AKKFLDKFDKIIYNNSKNYFEFAFDYNNFTTKAEGTRTKWVVCTHGDTRYRYNPQTKTSEEVNI |
| | | | | | TQEIKYLLIKHNIQYENGNCFKNKLIQAEDKKFYSKLLHLLAITVSLRHAKSGTDIDFILSPVADK |
| | | | | | NGVFFDSRKANDTMPKDADANGAYHIALKGLLALNKISNTPDDKLNKVDLKITNKEWLAFAQK |
| | | | | | KN |
| MG29 | 6451 | MG29-128 effector | Protein | Unknown | MIDDLTNQYSLQKTLRFELKPIGKTLKYIEERGLIEEDYKRSQEYKRLKEIIDLYHKDFISEALDS |
| effector | | | | | VVFDDLEEYERLFFQNRDERDEKSFAKIQENIRKVIRKAFESHPNWKHLFKKELLNKVLPYWEH |
| | | | | | DNITDSDREIIKNFQKFTTYLNGFHKNRENIYSSEAKHTAVAYRVVHENLPVFLQNKRLFETIGQN |
| | | | | | YPEIIEAAKEELDTQERLMGATVEAVFNLDYYQYLMSQKHIDIYNTILGGFTLDDGTKIQGLNEK |
| | | | | | INKFRQDNKLSKRELPNMKPLYKQILSDREALSWLPDSFENIQDMSEAIASFYNKNILHFKCCDG |
| | | | | | EVNLLEKLDEVFEESNEYDLDKIYIRNDKSLTDISQAIFGDFGVIKDALWDYHLSKNPKLEKKKNI |
| | | | | | DDIQAKYFKKSYFSVKQIEEALKFYGKEESLIEYFKGFGAYKNSEKRDLREEIKSAYSNWSSDRE |
| | | | | | NKDKIKELLQALLNLQHFLKVLYVKDELEKDIAFYAYFDIYFEALSLVIPLFNKVRNYLTKKPYS |
| | | | | | TEKFKLNFQNSTLLYGWDVNKESANLGILFEKEGLYYLGIMDKKHNKIFQDIKESNSSNVYRKIE |
| | | | | | YKLLPGPNKMLPKVFFSKRRIDEFAPSEELIAKYKEGTIIKKGEKFNLEDCIINLIDFFKNSIKKIIP |
| | | | | | DWKEFNFNFSPTREYQDISEFYKEVELQGYKLTFKNIDTEIVEQFIEEGKLYLFQIYNKDFSPYSK |
| | | | | | GVPNLHTIYWRMVFDENNLKDITYKLNGDAEVFYRKHSIEYNEKIWKEGHHVKELAGKFDYPII |
| | | | | | KDRRFALDKFQFHVPITLNFKAPNITAKEHNNLIRQKIKENADSIKIIGIDRGERHLLYLSLIDSSG |
| | | | | | KILEQYSLNEIVNSYNGKEHRVDYHKKLDKIEQERKSARLNWEAVEGIKELK |
| MG29 | 6452 | MG29-129 effector | Protein | Unknown | MSAQSALSTLINKYSLSKTLRFELIPIGKTKESIDRKGLLSQDVKRAQSYKEVKKIIDEYHKEFIEK |
| effector | | | | | SLINAKLKGLEEFSKLYYKLQKEDKDKKNIKKMQDNLREQISDLFKNNKKDKWNILFKEDLIKK |
| | | | | | ELPLFAKDDKQKNLINEFNKFTTYFTGFHKNRKNMYAEEEKSTSIPYRIIHQNLPKFLDNIRIFEKI |
| | | | | | KKNKINTDVIEKELSLFLNGIKINDIFSINFFNDVLNQKGITFYNTILGGVSEKDRTKIKGINEYVNT |
| | | | | | EYNQKQLDKKSKIPKLKQLYKQILSDTETASFVLEQFENDNQLLEKIEQFYNTELINYETEGKTQ |
| | | | | | SVFLQFEQLFKNMQNYDASKIYISNLSIANISKIIFGDWSIICNALAEWYDKHNTKGKKINEYKKE |
| | | | | | NFLKQDFSIQQIEDAVLEYKNDTLNKEINFLLNYFASFLNEKSKKNIIQRIETEYSKVKDLLNTDYP |
| | | | | | EKKKLASDKDNVSKIKAFLDSLMDFLHFVKPFNIKKDTGLEKEENFYSIYVPLFEQIDKIIPLYNK |
| | | | | | VRNYLTKKPYSTEKIKLNFENSTLLDGWDLNKESDNTSVVLRKDDLYYLGIMDKKHNRIFKELP |
| | | | | | SQNGNESSYEKMIYKLLPGPNKMLPKVFFSKKGKKQFKPSKKLLKKYEDGTHLKGDNFNINDC |
| | | | | | HNLIDFFKESIAEHEDWKQFDFKFSSTSSYKDLSNFYKEVEKQGYKITFQNISENYINQLIDEGKL |
| | | | | | YLFQIYNIDVQNK |
| MG29 | 6453 | MG29-130 effector | Protein | Unknown | MKNFTNKYSLSKTLRFELIPQGKTSEHIENKGLINQDEKRAENYKRIKSIIDDYHKDFIRQAMSNV |
| effector | | | | | QLTKLEIFADLYNAYNEKKKEESFKKEFEKVQSDLRKEIVAGFSSDNVKEIFSEIFGEKLIKELLE |
| | | | | | DWMRKQDCYSDEHYELLKDFKTFTTYFTGFNDNRKNIYTSEAHSTAIAYRLIHENLPKFIDNIKA |
| | | | | | YKKVKESRININKLEENLDSLLPGVVLDQVFSLDYFKYVLTQTGIDNYNLVIGELNLVGNLFNQK |
| | | | | | QEIKNNRIPKLKPLYKQILSDRQSTSFSLEAFEDDSNSTASQQVLDTINTYYRNSLICFYPTENSEPE |
| | | | | | NVLEEIKKLLEDLKTYDLDKIYLRNDAKITAISKKIFGDYSVFGSALNYYYKTVENPKFEEEYKK |
| | | | | | AKSQKAKENLENKQEKFVDKPYIAISLLQKALDIYIESLDDNHKLKDKYKPNCVADYFHTYFISE |
| | | | | | QNSNGKKFDFVSNINAKHSCIQGLLNDAYPLDKKLHQEKDDIAKLKLFLDALMEMLHFVKPLAL |
| | | | | | AKDSNLPKNDSFYGQFTTWFEQLDLLIPLYNKVRNYATQKPYSVKKIKLNFENSYFLSGWATEY |
| | | | | | SSKGGLVIIKENDFYLAIVDKKLSDDDVAFLTENSNLNLAQRVVVDFQKPDNKNIPRLFIRSKGEN |
| | | | | | YSPAVEKYNLPIGEVIDIYDSGKFKTEYRKTNPQDFKQSLVKLIDYFKEGFTKHESYKHYQFLWK |
| | | | | | DSKEYLDISEFYKDVESSCYQLSFENINFDNLLNLVDQEKFYLFKIYNKDFSKHNKGKPNLHTIY |
| | | | | | WKALFDEQNLANVFYKLNGQAEIFYRRKSIKEPTIHKAGEAIANKNPNNQKKESIFDYDLIKDRR |
| | | | | | YTVDKFQFHTPITLNFKAKGGDYINYDVLSYIKNNPDIKIIGIDRGERHLIYISLIDQKGKILKQESF |
| | | | | | NIISNDGHETSYHNLLATKEKERADARENWGVIENIKELKEGYISQVVHKIAKMMVENNAIVVM |
| | | | | | EDLNFGFKRGRFKVEKQVYQKLEKKLIDKLNYLVFKDKQPNEIGGLYNALQLTNKFESFQKLG |
| | | | | | KQSGFLFYVPAWNTSKIDPTTGFVNLFYAKYESVEKAQEFFGKFEDIRFNPSHNYFEFEVKKYSD |
| | | | | | FNIKAEGTRQDWLICTYGDRISTFRNSQKNNSWDNKEVILTDEFISLFEKYAIDYKSENLKNLIISQ |
| | | | | | TDKSFFESLLRNFKLTLQMRNSITNSEVDYLISPVQNNKGEFYDSRKADETLPKNADANGAYNIA |
| | | | | | KKGLQCLEQINKFEGENWKKLELDKANNSWLRFAQNKN |
| MG29 | 6454 | MG29-131 effector | Protein | Unknown | MKNQIILFSGFTNQYAISKTLRFELKPVGRTLEIIIEKKGLITQDNQRAEDYKEVKILIDEYIIKNFI |
| effector | | | | | EKSLDGFALNGLQEYYDLFIKTNLEEIDKKALDKEKDNLRKQIANRFKKHDKFKSLFAKELIKED |
| | | | | | LIGFVSEENKELVNKFKNFTTYFTGFHENRKNMYVADDKATAIAYRLIHENLPKFIGNIIIFEKIK |
| | | | | | KDAPDLVSQLNNVLSEMEEIIQGKTLEEIFSLDYFNETLIQTGIDLYNIVLGGRSEDGKDKIKGLN |
| | | | | | EYINLYNQKQTEKKNRQPKLKQLYKQILSDRDSVSFIAEEFKEDTEVLEAIEKFYQGELCNYESD |
| | | | | | GQAINVFNVSKKNLIGNLSSFDLSKVYLRNDRAITYISQQMFSDRSIVGNALQEYYRAQNPQKGK |
| | | | | | EKTENFEKRIDKWVKKSDYFDIQTVQEALSQCSIEAVKEKNKQNAIVNYFADMGLNEEIKMNLF |
| | | | | | DKVIADYKNVKDLLNTSYPENKKLGNQKGKDSDIEKIKTFLDSVMNIIHFIKPLNLKDESKEKDE |
| | | | | | TFYSLFLPLFDQLNKTILLYNQVRNYVTKKPYSTEKIKLNFENSTLLDGWDVNKEQDNTSVILRK |
| | | | | | DGLYYLAIMDKSNKKIFLNAPKADDKKDSFKKMNYKLLPLVNQQLPRVFFAKSRIEYFNPSSKIV |
| | | | | | QNYKNNTHKKGDTFNINDCHALIDFFKASLEKHEDWKHFNFKFSPTKTYQDLSGFYREVEQQG |
| | | | | | YKMTFENIPTDYINEMIEEGKLYLFQIYNKDFSPYSKGKPNMHTIYWKMLFDEQNLKDVVFKLN |
| | | | | | GQAEVFFSEKSIKDNIIVHKSNNSIENKNPDNPKKQSKFNYDIIKDKRYTIDKFQFHVPITLNFKAT |
| | | | | | GRDYINEDVNRFLKNNKEVNIIGIDRGERHLAYYMIINQKGEILEQTSFNIISSKHKENKYSTNYH |
| | | | | | ALLEKKEMARDKARKSWDTIGTIKELKEGYISQVVHKIAQLMVKHNAIVVLEYLNPGFKDSRK |
| | | | | | KVEKQVYQKLEKMLIDKLNYLVFKDYEANTTGGVLKALQLANKFTSFDRLGKQS |
| MG29 | 6455 | MG29-132 effector | Protein | Unknown | NCIAYQKITQKYSDEGFTAELVKCEQFFTPENYGICLTQGGIDVYNQLIGKKSDDTYGKGINQQT |
| effector | | | | | NEFRQKNSLKRNDVPLMTVLFKQLMSESERTFAIETIESDEELFSVVKDAYNTCINLVDGLSSLCL |
| | | | | | SNLTDDNYSSILIRSDGLSNLSQKIFGRWDIIDSALNLHKESIGQKNFDAKYSKVISLFDLQTICDTY |
| | | | | | TSTLDDSVITRNITFLDYYKSFDSSLINNAYLETESVLNSGGLDKDKTMPEKDTDFGGKGYQQLQ |
| | | | | | KIKQLLDAVNDAVHFYRPFLLEKEGKAIEADENNKEFYNEFLLYYRDLSCFPKVYDKVRNYATK |
| | | | | | KPYSKDKFLLNFDKPTLLDGWDVNKEESNLAVMLFKGGKYYLGIMINNRLFSDYSTIGTVSKNE |
| | | | | | QAYEKLVYKQVSGACKMFPKVFFANSNRELYKPSSAIEKIRNEKSHLKGGSENSKNKWIQFCIDC |
| | | | | | IDKQPEWKTYFKFNFKKPEEYPDVNSFYKDADAQMYSISFAKIPCEYVDKAVTNGELFLFELYNK |
| | | | | | DFSEHSKGRPNLHTLYWKMLFDESNLNNIMNNPDKGIFKLNGEAEIFYRKASLPDKPTHPANKPI |
| | | | | | TNKNPLNRKMQSTFEYDIKKDRRYMSDKFMFHCPLTINFRKEKVGQGQFNTKVNTAIEQNLED |
| | | | | | VNIIGIDRGERHLLYYTMINSKGNIIKQGSLNSMTADCGVTTDYRSKLNDKEKSRTAGRENWGQI |
| | | | | | ETIKELKQGYLSQVVHTLSQMMIENNAVIVLEDLNTGFKNGRKKVEKQVYQNFEKALIDKLNYL |
| | | | | | VFKEKAVGEKGSCLEGYQLTAPFESFTNLGKQSGFLYYVIPAYTSKICPKTGFVDFLKPKYESVE |
| | | | | | KSVELFEQFDKICFNAEADYFEFSFDFKKTGVDKGGKTKWTVCTFGKERYVYQGTDKEPKCIDV |
| | | | | | TEELKNLFSFYNIALADKKDLLPQILTVKEKNFWSSLIYTLRATLQMRYTKPGTEDANDYILSPV |
| | | | | | MDENGVFFDSRNAVENEPKNADANGAYHIAVKGQKLIASITAGRTTDSGKLNEWLEYVQKRG |
| MG29 | 6456 | MG29-133 effector | Protein | Unknown | METNFFKSFTKQYSLSKTLRFELKPIGKTIANIETKGLLAQDNQRAESYKKVKKTIDEYHKYFIE |
| effector | | | | | LALKAVKLTKLDEYYELYSKNKEDRDDAAFKKVKEDLRKEIVAVFTKGGFKEMFIRLFSKELIK |
| | | | | | EDIAAWISEHPKFAEDLKFVKEFDNFTTYFTGFNENRKNIYSAEDKSTAIAFRIVHENLPKFIENIK |
| | | | | | LFKTIKEKHANLDFSPILKEMEEVIQGVTLDEIFTLDYFNHVLSQNGIEFINFIIGGKTLKNGDKIK |
| | | | | | GLNEYINLYNQQQKDKNKRAPKFKQLYKQILSDRTSISMRFEEFENDSDLLETVEVFYQSELCEY |
| | | | | | ETEGKTIINIFEEIKTLCESIGTYNLEKIYLRNDSNLTNISQRLFGSFSVFRDSISFYYDTVVDTNFQT |
| | | | | | KYANAKTENQIKHLNDTKNKWNSDFISLGLLQKTLAKYIETLDADSEIRKIYTPTIITDYFKRHIIK |
| | | | | | KEVEEAKNNETIKKTTDVELFYSITGQYLGVKGLLNIEKTDNKTLAQEKEKVHQLKSFLDSILEL |
| | | | | | NHFVKPLFLTDDSISDKDDAFYSQFAPLYEQLNKLIPLYNMVRNYLTQKLYCTDKIKLNFENSTL |
| | | | | | LDGWDVNKEPDNTSVILRKDGLYYLVIMDKAGKKVFMDVPKIAYSGTFYEKMNYKLLPLVNQQ |
| | | | | | LPRVFFAKSRIEFFKPSEAIQENYKKETHKKGDTFNIKDCHALIDFFKASLAKHEDWKHFNFKFS |
| | | | | | PTKSYQDLSGFYREVEHQGYKMSFENIPTDYIDKMIEEGKIYLFQIYNKDFSAFSKGLPNMHTLY |
| | | | | | WKALFDENNLADVVYKLNGQAEVFFRKSSIEEKNKVIHKAHELLKSKNPNTPNNNNTFDYDLIK |
| | | | | | DRRYTVDKFQFHVPININFKASGSEIINAQTNDFLKNNRDVKIIGLDRGERNLIYLTLIDQKGNIIIQ |
| | | | | | ESLNTISNKERKIETPYHTLLNIKEKERDAARKSWNTIENIKELKEGYISQVVHKIAEMMVKHHA |
| | | | | | IVVMEDLNFGFKRGRFKVEKQVYQKLEKMLIDKLNYLVFKDAQPTQPGGLLNALQLTNKFESF |
| | | | | | KKMGKQSGFLYYVPAWNTSKIDPATGFVDFLKPKYENVEKAKAFFSKFDSIKYNTVKDYFEFAF |
| | | | | | DYKNFTTKAEGSKTDWIVCTHGDLRFRYNAQTKESEAVNVSQEIKKALKKHEITFEQGKDFKNL |
| | | | | | LIAKGGKEIFSALLHLLALTLSLRQTKSGSEIDFILSPVTNRKGVFFDTRNADEKMPIDADANGAY |
| | | | | | HIALKGLWCLQQISQSDDMKKVKLAISNKEWLEFVQNLCWH |
| MG29 | 6457 | MG29-134 effector | Protein | Unknown | MNFNNFTHQYSLSKTLRFELKPIGETADYIEDFKSQYLKDIVTQDQQRAEDYKVLKEIIDDYHRH |
| effector | | | | | YIEEKLSEPFDKKTGELFISEEDFENAFSYYQRFKEDPKDEKSKKDWLDTQSSLRKSVVKIFSDRK |
| | | | | | KRLFQKELITKELPAWLKEKGEWEEKKNVVENFNRFTTYFTGFNENRENMYSDEEKSTAISFRL |
| | | | | | MNENLPKFLNNCLQFGVIIEKHIDLDLKIESKLLQKMGVSNLNEVFQPSYFIQLFTQTGIDNYSEL |
| | | | | | LGGWTKENGEKIQGLNELINLHRQKYSIKAKGLPNFIGLYKQILSDRESSSFIPDQFENDKALLES |
| | | | | | LKIFVKEMAKTDGIFVNLKKAVTLLKEADLDKTFIKGGVEISKISQAIFGQYSIIRSAIYNYAETV |
| | | | | | MYPTPKTGKVSDVLEEKRKKYANNEDIFSIAELESMLTSYREQLEDEHPDKEITHCENSEHPIRT |
| | | | | | YFLNIIDNVKNDKDIELGKSIENVLPLLSLENLNKGKAGQTQTHLIQKMLDAFLAVTHAVKPLHL |
| | | | | | VKGRKPIEVPDIDMGFYADFSSAFEIYQQMIIGLYNKTRNHLTKKPFSTDKIKINFENPTLLDGWD |
| | | | | | ANKENDNSGILFKKDGNYFLGIMHPKHKNIFNYIKGINDIESEKRSLSKDELFDKIVDGNSDHYQK |
| | | | | | IVYKLLPGVNKMLPKVFFSGRRIDYFAPSTEVLKIRNTASHSKNGKPQKGFEKADFNLKDCHTII |
| | | | | | DFFKQSLEKHPEWKEFEFDFSPTSSYEDLSGFYREVEHQGYKMDFHPIKKSYIDQCIEDGKLFLF |
| | | | | | QIYNKDFSPYSKGKPNLHTLYWKALFDPENLKNVVAKLNGQAEIFYRKHSIIKDDRTIHRSNKSL |
| | | | | | QNKNENNPKKTSLFEYDIIKDKRYTVDKFQFHVPITLNFKAEGVTRENDKINRELSKSDDTHIIGI |
| | | | | | DRGERHLLYYSVINHKGEIVEQETLNTISTDQGFAVDYQQKLDKKEKARDKARKSWSTVENIKE |
| | | | | | LKAGYLSHVVHKLALLIVKYDALICLEDLNFGFKRGRFKVEKQVYQKFEKALIDKLNYLVFKDA |
| | | | | | KPNEPGHVLNALQLTAPFDSFKKLGKQTGILYYVQAAYTSKIDPVTGFINFLYPKYESLIKSKTFF |
| | | | | | ESMEGIRYNPNKDYFEFSFDYRKMTPNRDLKGYQTKWTACTFGEKRFKNIRNANGNWESVEVN |
| | | | | | VTEALKEILKNEDVDFNSNQDLRLAISKVKSTKFYKKLFKLLQITLSLRHSKMGTDEDFILSPVAD |
| | | | | | ENEEFFDSRDATENQPKDADANGAYHIALKGLWNFEQIRNWDGESRLNLAMKNVEWFAYAFQ |
| | | | | | KPFIK |
| MG29 | 6458 | MG29-135 effector | Protein | Unknown | MKNFTNIYPQSKTLKFELRPQGATLDNIHKSGLIDQDETLKADYQAVKKMIDEYHKVVIDESLT |
| effector | | | | | NFKLTGLPAYEELYYKDRTEAEDKTFEKIQSNLRKQVIIKAFGENKRYKSIFKKELIQKDLPDFV |
| | | | | | NKEEEREQISRFYHFTTYFTGFHENRKNIYTAEAKATSVCNRLIHENLPKFLDNRKTYLNYISNFG |
| | | | | | DLDMSQAEEDLQEVLGGIKVGELFSLDFFNHTLTQQDIDIYNIALGGRSVEGGKKIQGINECINLY |
| | | | | | RQNNQLKARQLPNIKPLYKQILSESESGSFLLDKFEKDEDLFDGLRNFYQGLNSFNYKGEQDKST |
| | | | | | FIELMNLFGRFSESDMTRVYLRNDASLSRLSKKLFGDWSLIVSALRYYYDAEVNPLMGKKATNK |
| | | | | | YIKEKENWLNKSRDFSIDIINKSLLRYGTINETVNSQFTDDIIFEHFSSFMIEEKNLLNTVAENFML |
| | | | | | VSEVLSRGSLDKDQNKKKKEIKTIKTFLDNVLDLLHFIKPLSVQHVGAEKDEGFYSDFDVLYDQL |
| | | | | | SQVIPLYSKTRYYLTKKPYSVAKFKMNFKNNTLLDGWDVNKETANKGVLLQKEGLFYLAIMNK |
| | | | | | DHSKSFYNIMDTGDTTGYQKMDYKLLPGPNKMLPKVFFSVKNLGFFNPSEKVLRIRNTSSHSKN |
| | | | | | GNPQEGFDKADFSLNDCHSLIDFFKASLDKHADWKKFAFDFSPTQSYNDISEFYREVENQGYKIT |
| | | | | | YTNISDEYIHELVNEGKIYLFQIYNKDFSPFSKGKPNLHTLYWRALFDEKNLEDVVYKLNGQAEV |
| | | | | | FYRKKSIEYSEEKWIQGHHYDQLKDRFAYPIIKDKRFAFDKFQFHVPITMNFKASGSPVINMKVR |
| | | | | | EYLKTNPDVKIIGLDRGERHLLYLTLIDQNGNIEEQYSLNEIVNSYNGKVHKKDYQQLLHEKEG |
| | | | | | DRKKARENWETIETIKELKEGYLSHVVHKIVNMMVEHNAIVVMEDLNFGFKRGRFHVEKQIYQ |
| | | | | | KFEKMLIDKLNYLVLKDTQDPKAPTGLLNALQLSTKFESFQKLGKQSGFVFYLPAYLTSKIDPAT |
| | | | | | GFVNQLRIKYDSVVKSQAYYRQFDRIVYNTTTDWFEFGFRYVNFGNTTPSVRQEPWTICTTHHP |
| | | | | | RYAWNKNSNMGKGGTEEYNITEELKKLFDHHNIAYEEGTDLIESIASNTGTDFFKRLNKLLNVT |
| | | | | | ASLRHNNGKKGKEERDFILSPVANSEGAFFNSLEADETQPENADANGAYHIALKGLWALQSIRK |
| | | | | | TDTDRMAKLNLVVSNEEWLNFAQAKQYRSQSSS |
| MG29 | 6459 | MG29-136 effector | Protein | Unknown | MFQAFTNQYPVLKTLRFELKPINETTGYLEDFKSAYLKKVVGQDEKRASDYQAIKKIIDNYHRH |
| effector | | | | | YIDQCLVAPCDEKTGELFITPEQMEDAFSYFQAYREKPDEKGGKEGWMKTQKTLRANLVKAFK |
| | | | | | DNGSLFKEDLIKRLLPEWLQQQGTWEECREAVESFSKFTTYFTGFHENRKNIYSSEDHSSAISNR |
| | | | | | LMNENLPRFFNNCIQYTQLREKYPELGLNAPANILQALNASTLDKVFQPGFYLNLFTQRQISHYQ |
| | | | | | ELLGGKTTDTGEKQQGLNEQINLFRQKTGLRPRELPGFTPLYKQILSDVESHSFVPEAFASDRQL |
| | | | | | LDALAAFIMQMDEKMPALMEQLDGLEQADQTKTHIKAQSLGTVSQALFHSWGIIGVALARHAE |
| | | | | | SLFPANAKGVVTKAIEEQREKYGKQETYPLAEVDQVLTGYISTLEQDDPLHQQLKALSTPDKPL |
| | | | | | LAYFRQAAIQTQVSISPLKKQLAELLILPELSKDRQLPKDDQETGGQGFQQVQRIKALLDAYKAL |
| | | | | | VDVFKPLHLVKGRKAMEIPDLDNGFYSMFEETYATCEQPVVALYNKTRNYLSQKPFSADKIKLN |
| | | | | | FEDLTTTLLNGWDVNKETANGNILLEKDGLYYLGILHPKHKRLFDYVKDIDDFGNAKMEQKKD |
| | | | | | VLRTSLEAESGELGYRKIVYKLLPGANKMLPKVFFSASRIGFFQPSSDIQRIRDTASHSKNGTPQP |
| | | | | | GHKKADFSLSDCHAMIDFFKASIEKHPEWREFGFQFSPTHTYVDMSGFYREVEQQGYRIDFHRI |
| | | | | | KASYIDECVKDGKLFLFQIYNKDFSPHSKGAPNLHTLYWKGLFDPENLKDVVIKLNGEAEIFYRK |
| | | | | | HSIRQHDRIIHRAGQPVANKSGGNPKRHSLFEYDLIKDRRYTQDKFQFHVPITLNFKAAGVNAFN |
| | | | | | DRINTTLAGQQGTHVIGIDRGERHLLYYTVINPRGEIVEQGTLNTVEVNVPTTAERAGYQYGVD |
| | | | | | YQKMLDAKESERDKARTSWAGIENIKELKEGYLSHIVHKLAKLITRYNAIVCLENLNAGFKRGR |
| | | | | | FKVEKQVYQKFEKALIEKLNYLVFKDAKPGEAGHFLRAYQLTAPFEGENKLYQQTGILYYVRA |
| | | | | | DYTSKIDPATGFIDFLRPNYESVEKSAAFFRQFDAIRYNVAKDYFEFSFDYKKFQTRQRLESYPTR |
| | | | | | WTACTHGDTRYHNKRDKNGQWHTETINVTEQIKALLQQENIGFEQGQDIREAIAGSKDSPFFKS |
| | | | | | LLFLLRLTLSLRHSRTGTDDDFILSPVANELEEFFDSRKAGQNLPKDADANGAYHIALKGLWNLQ |
| | | | | | QIRQHDWSVAKPQPLKLNLSNEEWFGFVQGKPYLKG |
| MG29 | 6460 | MG29-137 effector | Protein | Unknown | RLFGSFSVFRDSISFYYDTVVDTNFQTKYANAKTENQIKHLNDTKNKWNSDFISLGLLQKTLAKY |
| effector | | | | | IETLDADSEIRKIYTPTIITDYFKRHIIKKEVEEAKNNETIKKTTDVELFYSITGQYLGVKGLLNIEK |
| | | | | | TDNKTLAQEKEKVHQLKSFLDSILELNHFVKPLFLTDDSILDKDDAFYSQFAPLYEQLNKLIPLYN |
| | | | | | MVRNYLTQKLYCTDKIKLNFENSTLLDGWDVNKEPDNTSVILRKDGLYYLVIMDKAGKKVFMD |
| | | | | | VPKIAYSGTFYEKMNYKLLPLVNQQLPRVFFAKSRIEFFKPSEAIQENYKKETHKKGDTFNIKDC |
| | | | | | HALIDFFKASLAKHEDWKHFNFKFSPTKSYQDLSGFYREVEHQGYKMSFENIPTDYIDKMIEEG |
| | | | | | KIYLFQIYNKDFSAFSKGLPNMHTLYWKALFDENNLADVVYKLNGQAEVFFRKSSIEEKNKVIH |
| | | | | | KAHELLKSKNPNTPNNNNTFDYDLIKDRRYTVDKFQFHVPININFKASGSEIINAQTNDFLKNNRD |
| | | | | | VKIIGLDRGERNLIYLTLIDQKGNIIIQESLNTISNKERKIETPYHTLLNIKEKERDAARKSWNTIEN |
| | | | | | IKELKEGYISQVVHKIAEMMVKHHAIVVMEDLNFGFKRGRFKVEKQVYQKLEKMLIDKLNYLV |
| | | | | | FKDAQPTQPGGLLNALQLTNKFESFKKMGKQSGFLYYVPAWNTSKIDPATGFVDFLKPKYENV |
| | | | | | EKAKAFFSKFDSIKYNTVKDYFEFAFDYKNFTTKAEGSKTDWIVCTHGDLRFRYNAQTKESEAV |
| | | | | | NVSQEIKKALKKHEITFEQGKDFKNLLIAKGGKEIFSALLHLLALTLSLRQTKSGSEIDFILSPVTN |
| | | | | | RKGVFFDTRNADEKMPIDADANGAYHIALKGLWCLKQISQSDDMKKVKLAISNKEWLEFVQNL |
| | | | | | C |
| MG29 | 6461 | MG29-138 effector | Protein | Unknown | AETIEIARGLFSEVINLTAIHALADNSENIYINSSALANLSHRFCDDWNLIYRACEAKMIKLSGKQK |
| effector | | | | | KGLENKLKMAIPMSELQNIIEEYIATLDEELKLSYNNIPVLCDYFQNPPLDDFESATLKFEQIVKT |
| | | | | | TMSRTDLIQAIKEVLDKAMEVVRFFKPLYLFKGRSPLEVPDRNEDFYNEFERLYAELNLISKIYD |
| | | | | | RVRNYATKKQFSQDKIKLNFNNPTLLDGWDLNKEQDNLCVILIRDGNYYLALMNRDYRRLFDL |
| | | | | | KNDEVRNKALGKAGDHCYSKLEYKQVTGASKMLPKVLFAATNSDLFKPSQEILDIRKAGSHKK |
| | | | | | EAGNIEALHKWIDFCKQSIATHPEWNDHFDFKERSTSEYSELTEFYNDFDRQAYKIKFVDIKVEYI |
| | | | | | DQLVKEGKLYLFQIYNKDFSPYSKGRPNLHTTYWRMLFGNENLANITMDPDRPIFKLNGEAEIFF |
| | | | | | RKASLEKQITHAKGQPITNKSKKDNGKESESIFEYDLIKDKRYTEDKLFFHCPITINFRAPGTTVG |
| | | | | | SFNRKVNYFVERNPEVKIIGIDRGERHLLYYTVIDQKGNILEQGSLNQIHNSYTSAGRVVEHNINY |
| | | | | | RDLLHEKEKGREEARKNWETIENIKELKAGYLSQIVNLLSNLMIKYNAVLVLEDLNAGFKRSRI |
| | | | | | KVEKQVYQKFEKAMIDKLNYLVFKELPPGSSGHYLNGYQLTAPFTSFRDLGRQSGFLYYVYPSY |
| | | | | | TSHICPKTGFVNLLNTRYESIEKAISFFEKFNSIKYNPGSDYFEFDFDYASFGKDVARSQWCVCTA |
| | | | | | GEKRYYYANHDKTSRECNATQQIKELLDKYNIEYIRGKDLLPELIKMNDKGFLNGLMFLLGVVL |
| | | | | | QMRYTVSGTSNDDDFILSPVMDEQGQFFDSRSAATSEPQNADANGAYHIALKGLKMISSISDGKL |
| | | | | | KTVNKNERQDWFAYVQNKMYR |
| MG29 | 6462 | MG29-139 effector | Protein | Unknown | MQTIFNDFQGLYSLSKTLRFELKPVGKTKELLEDFYNHCQDNPIAVDEQRIGHYPKMKDLIDDY |
| effector | | | | | YRFFIDKILSPTIYSAEEMQNAYELYCAAKSDNKNNQKDYLAAKKILRKKLAKCFTDQKTIYGLN |
| | | | | | EYSKLFGQEQMPLNLWLKCRLDNHQITEAEYQENMATIKAFERFTTYFTGFKENRENLFVEDD |
| | | | | | KASAIANRVIEENMEKHFFNCRTLTAMVEKQPQLAKELAEFLPLFTPEHYCKCINQQGIDSYNQ |
| | | | | | AIGRKIDCEGEKGVNQILNEYKQNSNLRAKDLPMLTTLFKQLLSKKSDCLFNETITTDQEMLQL |
| | | | | | TQYCYNKAKKQLNELQKIMADYLTDENLQHIYLKKASLNALSHRFLGEWDTVRNAYLNHYQQ |
| | | | | | LTKKEQNAFDKQTKEVIGLDLLQTIISNYLPNLESAPSVSQLAIYLGSIDLEPLNKAYAEAASVLAL |
| | | | | | EKLDDDKRLPENINDEGGLGYQQIMLVKCLLDKIIDAKDFYKPFLLEQGHKPLKVDNNNELFYA |
| | | | | | QFVSAFYELDSLCQKYNFIRNYATKKATSTDKFKLNFSKPTLLNGWDLNKEEANSNVLLRKNDK |
| | | | | | YFVGILINKKLFADPAKYICEDEAEHYQKMLYKQVSGVNKMFPKVFFAAKNQNIYKPSAQILQIK |
| | | | | | EAKSHLKEANNPSAKNAWIDFCKDSIKKSEWPQYFNFKFKPANEYPDVNSFYQEADAQMYSLAF |
| | | | | | QNVAADYVTQAVENDELYLFEIYNKDFSACSKGKPNLHTMYWQMLFDEQNLQNIADSAEKPVF |
| | | | | | KLNGEAEIFYRKASLVDTVTHPAGVPIANKNPLSSKMTSTFTYDIKKDRRYMQNKLYFHCPITIN |
| | | | | | FRKNTMPQTVFNYKVNTFLQDNPNVNIIGIDRGERHLLYYSLINQKGQILKQGSENTITAAGNTT |
| | | | | | DYHNLLDKKEHQRTQARQDWATIERIKDLKSGYLSHVIHELATLMIDNNAVVVLESLNKGFKTG |
| | | | | | RQKIEKQVYQKFEKALIDKLNYLVFKDSAPNQAGHCLSGYQLTAPFESFDKLYNQSGFLYYVVP |
| | | | | | AYTSKICPKTGFVNLFNSSYTNYQSVNASVEFLKKFETIKYNTQADYFEFRLDYKNFFEAKGKTT |
| | | | | | WTICTHGQERYHYNPKDKKYKCIDVTQQLKELFTNYGIDYQSENDLRQEICAQTEKSFFSGLLF |
| | | | | | YLKLTLQLRHTNGGTDDINDFILSPVADSNGCFFDSRQAQLTEPKNADANGAYHIALKGLKLLK |
| | | | | | GLDEGRTPSTKNEKAQWLEFTQQHQYLEA |
| MG29 | 6463 | MG29-140 effector | Protein | Unknown | MLFKKFTNQYPLSKTLRFELKPVGETADYIEDFKSQYLKDIVAQDEQRAEDYETIKETIDNYHQH |
| effector | | | | | YIEEKLSEPIDKKTGELFISGEDFENAFSYYQCFKENPKDEKSKKNWGDTQTSLRKNLVKSFYDR |
| | | | | | KKQLFQKELITKELPAWLKEKGEWEEKKHVVENFNRFTTYFTGFNENRKNMYSAEEQSTAISF |
| | | | | | RLMNENLPKFFNNCIQFGVIIEKHIDLDLKIESKLLQKMGVSNLDEVFQPSYFIKLLTQTGIDNYS |
| | | | | | ELLGGWTKENGEKIQGLNEIINLYRQKNAIKAKYLPNFISLYKQILSDRETSSFIPDQFESDKDLL |
| | | | | | QSLSVFINEMAKEDGLFKKLEESITLLNECDLHKTFIKNGIEITNISQSIFRNYSILKSAIFHHAETTI |
| | | | | | YPTPSNGKISEALKEKRKKYVNKQNVFSIAELEVMLSVYYNQLDDDNPIRETFKNGITSNNLLQS |
| | | | | | YFLGAINAVKNAKDTCLTEKMEKVLPLLSLDRLSKGEKGEEQIRHIQAMLDAFLAVSHVVKPLH |
| | | | | | LVKGRKPIDVPDVDMGFYTDFSTSFEAYDQTVIMLYNKTRNHLTQKPFSKDKIKINFENPTLLDG |
| | | | | | WDTNKEKDNSGVLFEKDGNYYLGIMHPRHKKIFDYNKGINDLESVKRSQTKDKLFDKIVDCSQN |
| | | | | | HYNKIVYKLLPGASKMLPKVFFSKGRIEFFSPSNDVIRIRNTASHTKNGKPQEGFKKAVFNLNDC |
| | | | | | HTIIDFFKTSIEKHPEWKEFGFDFSPTSSYEDLSGLYREVEHQGYKMDFHPIKESYINQCIEDGKL |
| | | | | | FLFQIYNKDFSQYSKGKPNLHTLYWKALFDPENLKDVVAKLNGQAEIFYRKHSIKKDERTIHFA |
| | | | | | NESLKNKNENNPKKTSSFEYDIIKDKRYTVDKFQFHVPITLNFKAEGVSRENDKINHELAKSNQT |
| | | | | | HVIGIDRGERHLLYYSVINPKGQIVEQGTLNTISTDQGYEVDYQQKLDEKEKARDKARKSWTTV |
| | | | | | ENIKELKAGYLSHVVHKLALLIVKYNAIVCLEDLNFGFKRGRFKVEKQVYQKFEKALIDKLNYL |
| | | | | | VFKDAKPNEAGHVINALQLTAPFDSFKKLGKQTGILYYVQASYTSKIDPVTGFVNFLNPKYESLL |
| | | | | | KSKIFFESMDGIRYNTDKDYFEFLFDYRKMTPNRELKGYQTQWTICTHGNKRFKNIRNEYGKW |
| | | | | | ESVEVNVTEELITLLTNEGIEFQSGKDLKNAITTAKRTKFYKSLFKLLQLTLSLRHSKTGTDEDFI |
| | | | | | LSPVANEAGQFFDSRNATEIQPKDADGNGAYHIALKGLWNLQQIKAWDGESKLKLAMKNVDW |
| | | | | | FTFAQKKPYLKN |
| MG29 | 6464 | MG29-141 effector | Protein | Unknown | MKLNKFTIIQYPISKTLRFELKPVGETADYIEDFKSQYLKDIVIQDQQRAEDYESIKVIIDEYIIRIIY |
| effector | | | | | JEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSHEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNDLLKSL |
| | | | | | GKFIKEMVKEDGLFKKLEDSIKLITDADLHRTFIKNGVEITKISQSIFGNYSILKSAIYHHAESVLY |
| | | | | | PDPISGKISEALKEKRIKYVNKQVVFSIAELETILSNYASQMTDDNPDKEIIAQCENSEHPIRTYFL |
| | | | | | NAIENVKNDKDIEFGKAIENVLPLISLENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEEGKLFLFQIY |
| | | | | | NKDFSPYSKGKPNLHTLYWKALFDTENLKDVVAKLNGQAEIFYRKHSIKKDERTIHRANKSLQN |
| | | | | | KNENNPKKTSLFEYDIIKDRRYTVDKFQFHVPITLNFKMEKMITQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDSRNATKAQPMDADGNGAYHIALKGLWNLEQIRNWDGESRLNLAMKNVDWFSFAY |
| | | | | | QKPFKK |
| MG29 | 6465 | MG29-142 effector | Protein | Unknown | MKLNKFTHQYPISKTLRFELKPVGETADYIEDFKSQYLKDIVIQDQQRAEDYESIKVIIDEYHRHY |
| effector | | | | | IEEKLSEPFDKKTGELFISGDEFENAFSYYQRFRENSKDEKARKEWIEVQNSLRKSLVKVFSDRK |
| | | | | | KRLFQKELITKELPSWLKEKGEWEDKKNVVENFNRFTTYFTGFNENRENMYSHEEQSTAISFRL |
| | | | | | MNENLPKYFNNCIQYHKAIESYDGLSFTVNPDLLNEMGVSILSEIFQPSYYIQLFTQSGIDKFTELL |
| | | | | | GGFTKENGEKVQGLNETINLFRQQKAIKAKAFPNFISLYKQILSDRETSSFIPDQFENDNELLKSL |
| | | | | | GKFIKEMVKEDGLFKKLEDSIKLITDADLHRTFIKNGVEITKISQSIFGNYSILKSAIYHHAESVLY |
| | | | | | PDPISGKISEALKEKRIKYVNKQVVFSIAELETILSNYASQMTDDNPDKENIAQCENSEHPIRTYFL |
| | | | | | NAIENVKNDKDIEFGKAIENVLPLISLENLNKGKDGQAQTHKIQKMLDAFLAVTHAVKPLHLVK |
| | | | | | GRKPIDIPDMDMGFYAEFSKAFENFEQLVITLYNKTRNHLTKKTFSTDKIKINFENPTLLDGWDA |
| | | | | | NKEKDNSGVLFEKDGNYYLGIMHPKHKNIFNYIKGINDIESEKRSLSKDELFNKIVDGESEHYQKI |
| | | | | | VYKLLPGVNKMLPKVFFSGRRIDFFAPSTEVLKIRNSASHSKNGSPQKGFEKEDFNLKDCHTIIDF |
| | | | | | FKKSIEKHPEWKEFEFEFSPTSSYEDLSGFYREVEHQGYKMDFHPIKKSYIDQCIEEGKLFLFQIY |
| | | | | | NKDFSPYSKGKPNLHTLYWKALFDTENLKDVVAKLNGQAEIFYRKHSIKKDERTIHRANKSLQN |
| | | | | | KNENNPKKTSLFEYDIIKDRRYTVDKFQFHVPITLNFKMEKMTQFAHNEKVNQMIVKSENTHVI |
| | | | | | GIDRGERHLLYYSVINPKGHIVEQGTLNTISTDKGYEVDYQQKLDSKEKARDTARKSWTTVENI |
| | | | | | KELKAGYLSHVIHKIAFLIEKYNAIVCLEDLNFGFKRGRFKVEKQVYQKFEKALIDKLNYLVFKD |
| | | | | | AKPNEPGHVLNAFQLTAPFDSFKKLGKQTGLLYYVQASYTSKIDPVSGFINFLYPKYESLLKSKIF |
| | | | | | FESMDGIRYNADKDYFEFSFDYRKMTPNRNLEGYQTKWTACTFGEKRFKNIRNAHGNWESVEV |
| | | | | | NVTEALKKILKNEDVDFKSGHDLRFEISKVKSTKFYKKLFKLLQITLSLRHSKTGTDEDFILSPIV |
| | | | | | DENGKFFDSRNATKDQPMDADGNGAYHIALKGLWNLEQIRNWDGESRLNLAMKNVDWFSFAY |
| | | | | | QKPFKK |
| MG29 | 6466 | MG29-143 effector | Protein | Unknown | MIYNVKLKKLTNSSDVFSLFSRKYSLSKTLKFELKPTAETKQHLQDFIVSDTKRAKEYKELKKIID |
| effector | | | | | EFHKDYIELTLSHKNILDKDKLTDFCKLWSDNNLLDEIKEKHNLKEKTKEEVIKKMEQQFRKDI |
| | | | | | VEQFKTLNPLLERFFKTADEEQLLGFLKKLDNKLWTKFKEKKEKALLDEYLQEPIKQKNIEKSI |
| | | | | | LFSSELIKYLLPVWLKNSQLTDKEDKQKIIKNFGRFTTYLTGFNENRKNMYSVEEQGTAIAHRIIN |
| | | | | | ENLMKFLGNLQAYEKIKSSHPELQQSFKNMKSDFKEEFDYFSLENIEDLFKPEFFNSCLSQKEIDF |
| | | | | | YNTLLGGKTVEDGKKLQGINEYINLYRQKKKSEDIDIKIKYSNKNLPTMELLYKQILSDRESHSFI |
| | | | | | LAEFENKKELLEALKSFWSVLFEEKTYQDYFHTKKKTSLWNNLHSLLTGRSYYDLEGAYFKSSE |
| | | | | | LNKLSHNLFKDYRIITEALNENYDKIKVTLESYKKVLKKEDKKEFEKNIKSLKGFQTEWKKILSF |
| | | | | | EKNKEELKFSDEILASYFEFPIDKKTKKKNQKDFYSLQEIKDHIELYSKESDELKEDLDNLKKKL |
| | | | | | KDYKKDNIISSWFKYQFESKQNLDFFLQRNHEKENEDKREQNKNSLFSHIENHYQKIQNLSLDEK |
| | | | | | EFKKEEVEDIKFFLDLILHVLHLMKPFYLEGKASNLLDKGINNEIEVLYKKLKPIEKLYNQTRNYI |
| | | | | | AKKKSRYNKVKINFEDSTLLDGWDLNKEKDNLAVLLRKKDAVIGWKYYLGVMNKTTKDLFDY |
| | | | | | HIKLDDSEKVKQKKEELQDLILHRENDGNFYEKMNYKLLPDPSKMLPKVFFSKKNLDSFKPSEEI |
| | | | | | VRIRDNKTYAKNGGQDFSKADCHKFIDFYKESLKKHKEWNDFFKFNFSPTSQYNDISDFFQEVK |
| | | | | | NQGYNLQFDKIKSSYIEEKVKTGELFLFEIYSKDFSTKSKNRKNSKDNLHTIYFKGLFEKENLKDT |
| | | | | | VLKLNGKAEIFYRKATKKFNITHKKNTELENKNKNNPNKHSIFNYDLIKDKRFTEDKFFFHFPISL |
| | | | | | NFNSKGMKSYLFNQEVLKCLKGNKEVNIIGIDRGERHLAYYTIINQKREVLSQGSFNKMESSYKD |
| | | | | | NSGKEVKIEKDYHELLESKEKERDKSRKEWNKIENIKELKSGYLSYLVHKISKLMIEHKAIVIFED |
| | | | | | LNLGFKRGRFKFEKQVYQKLEKALIDKLNYLVFKDKKSSELGGYLNAYQLTAPFESFQRMGKQ |
| | | | | | TGYLFYVPAYYTSKVCPLTGFINLIYPKYKNVKESQQFFEKFDRIYFDKNKNYFVFEYQDKKVNP |
| | | | | | SKKTESIETLWKVCTHGEERYKWDVKDRKMIEVNVTENLKKLFEEHKIEYRQIADLKSTITKQE |
| | | | | | KKDFFSKLIDGLKITLQLRHINPDSKDEKEKDFILSPVADESGRFFDSRKAKEGEPKNADANGAY |
| | | | | | HIALKGLRTLENITSDKKDKLKLQAITNKDWFSFLKENSNKKIPKVG |
| MG29 | 6467 | MG29-144 effector | Protein | Unknown | MGMIGDQFIGQYSLQKTLRFELRPIGETQKLLQDFKEEVQGNLLEYDAERARAYPMVKKVLDD |
| effector | | | | | YYRYFIDQVLSGFAFDSQTINEVYEMYKKAKKDAEAAKEYAVHTKKLREQLSAAFKAPITYYML |
| | | | | | DKYEHLFNRNRESRLFEWLDIRFENDHLTENEYDEIKDVLDKFDKFTTYFTGYKENRANLFVAD |
| | | | | | EKATALAYRVVNENMPRFFENCIRMENIKKRHLDLYKLLDSFEGYFVPQAYANIICQPAITDYNKI |
| | | | | | IGRPTQNPDEKGVNSIINEYRQKNQIKNRELPMMAQLYKQLLSDRITVFLDPVINNDEEMQSIVA |
| | | | | | ETIEIARGLFSEVINLTAIHALADNSENIYINSSALANLSHRFCDDWNLIYRACEAKMIKLSGKQKK |
| | | | | | GLENKLKMAIPMSELQNIIEEYIATLDEELKLSYNNIPVLCDYFQNPPLDDFESATLKFEQIVKTT |
| | | | | | MSRTDLIQAIKEVLDKAMEVVRFFKPLYLFKGRSPLEVPDRNEDFYNEFERLYAELNLISKIYDR |
| | | | | | VRNYATKKQFSQDKIKLNFNNPTLLDGWDLNKEQDNLCVILIRDGNYYLALMNRDYRRLFDLK |
| | | | | | NDEVRNKALGKAGDHCYSKLEYKQVTGANKMLPKVLFAATNSDLFKPSQEILDIRKTGSHKKE |
| | | | | | AGNIEALHKWIDFCKQSIATHPEWNDHFDFKERSTSEYSELTEFYNDFDRQAYKIKFVDIKVEYID |
| | | | | | QLVKEGKLYLFQIYNKDFSPYSKGRPNLHTTYWRMLFGNENLANITMDTDRPIFKLNGEAEIFF |
| | | | | | RKASLEKQITHAKGQPITNKSKKDNGKESESIFEYDLIKDKRYTEDKLFFHCPITINFRAPGTTVG |
| | | | | | SFNRKVNYFVERNPEVKIIGIDRGERHLLYYTVIDQKGNILEQGSLNQIHNSYTSAGRVVEHNINY |
| | | | | | RDLLHEKEKGREEARKNWETIENIKELKAGYLSQIVNLLSNLMIKYNAVLVLEDLNAGFKRSRI |
| | | | | | KVEKQVYQKFEKAMIDKLNYLVFKELPPGSSGHYLNGYQLTAPFTSFRDLGRQSGFLYYVYPSY |
| | | | | | TSHICPKTGFVNLLNTRYESIEKAISFFEKFNSIKYNPGSDYFEFDFDYASFGKDVARSQWCVCTA |
| | | | | | GEKRYYYANHDKTSRECNATQQIKELLDKYNIEYIRGKDLLPEIIKKNDKGFFNGLMFLLGVVL |
| | | | | | QMRYTVSGTSNDDDFILSPVMDEQGQFFDS |
| MG29 | 6468 | MG29-145 effector | Protein | Unknown | MKEFTNKYPSQKTLRFELQPQGKTRDYIDKNGILLRDKERNESYQEMKKTIDRFHKYFIDLALS |
| effector | | | | | DVKLSHLSQFKDLYLSVEMSEQQKKTIDEVKTKLRKEVAAAFRTGDAKSIFSLLDKKELITKLLK |
| | | | | | EWAEKERINNFYFDENFEKFTTYFKGFHQNRQNMYSDEAKATSIAYRSIHENLPRFIDNILIFQKL |
| | | | | | LQKEEIASHLHDICIGLEGYLNVNSIPEMFEINYYNTVLTQRQIEVYNAVIGGKTFEDGVQIQGVN |
| | | | | | QYINLYNQRCDHKDRVPPLKQLYKQILSDRESLSFLPDAFEEASQVFKSINQFFENNLIQFKVNDL |
| | | | | | EPVNILENVSKLMTELAFYDLKHIYVNNTFLSDISQKIFRNYAVIKEALNYQYESKIDPFFSEKYAK |
| | | | | | AKAENSREKLEKAKKNYTEKSYISLYEIQIALNNYMLTLEEDCIEKKSFTQTCLIDYYRSVFTHNT |
| | | | | | TEGEIDIITSIYRSYEELKEISSIDYPKNTPLSQDKSSIAKIKSLLDNVMELFHCIKPFAVPEEEISDK |
| | | | | | DGHFYNNFLVYYKSIKEIIPLRHKVQSYVTQKPYSLKKFKVNFDNSYFLTTWPFSYENKGGVIIR |
| | | | | | KEDLFYLAIINCSVKDLANYKLNDCSIANSAERIIIDTQKPDNKNIPRLFIRSKGDSFAPAVTEYDLP |
| | | | | | INDIIDIYDNGKFKTEHKKLDPEEFKSSLTALIDYFKLGLSRHNSYKHYKYQWKPSDCYNDISEFY |
| | | | | | NDTVNSCYQIKTEKINFDKILELVSEGKVYLFKIYNKDFSPNSKGQPNLHTMYWRALFDENNLKN |
| | | | | | VIYKLNGGAEIFFREKSISKENEIVHYANQPIKNKNPHAIKREVILPYDVIKDKRFCIDKFQLHIPIT |
| | | | | | LNFKATGKGQLNFDVLQYLKDTPQNQVKVIGIDRGERHLLYLTMIDHEGHIIMQESLNTVKSDN |
| | | | | | YPIETQYHDLLAQKEEDRNKARSNWDSIENIKELKEGYLSQVVHKLAKLIVENNAVLVMEDLNI |
| | | | | | GFKRGRFKVEKQVYQKFEKALIDKLNYLVFKDKKVEEEGGLFKALQLTEPYTDFLKYKKKQC |
| | | | | | GFLFYVQAWNTSKIDPTTGFIDMLKPKYKNIPEAQEFFRKFISIKYNSDKDYFEFHFDYRNFPRSI |
| | | | | | DSHKNDWTVCTYGKERYTWNRSLNQGKGDYEIWNVTEKIKELFNIEGIEYRSGHNLTEAIANSE |
| | | | | | SKSLLSMLMKSLSVVLAMRYSSSKDNRDFILSPVANDKGVFYYSEEADQYLPKDADANGAYNIAR |
| | | | | | KGLLLLDRIRSSADLKSLKLDISNKEWLMHAQK |
| MG29 | 6469 | MG29-146 effector | Protein | Unknown | AISQQLTGSWHTINDWIADVSDNKQLSQIKKQSVFSILELEQYFGIKIEGLNFYETHAKEEHKSDQ |
| effector | | | | | SLLKFFLKQFNEKFVEIDKTWHELSQSHVLELEQLDKKRYKAEQKGFQQVALIKAFLDSCNNLN |
| | | | | | YFIKDWTLKTSKDKYEDCYNFINSILDEFSIYHLYNKVRNYIAKKPFSTEKVKVTFEKSTLLDGW |
| | | | | | DRNKETQNLGILFTRNNQYYLGIMTSESNNLFNYDKLESDSIKKAELKSKTYNSCIASSSEDSYQK |
| | | | | | INYKLLPGPNKMLPKVFFAKSNIEFYNPSDEIITIKEKKLYSKAEIEKHGIANLHKYIQFCIDSLAK |
| | | | | | HPEWSVAYGFRKDSFKPVNTYQSIDEFYKDVEDLGYKITFDNIKKSYIDEKVENGELYLFKIHNK |
| | | | | | DFSKYSKGNKNLHTLYWQGLFEKDNLKDVVIKLNGEAEIFYRPSSIKLSETTIHKKGYEIKNKNV |
| | | | | | IKDKKISKFDYDIIKDKRYTQDKFFFHCPITLNAKANSNPGKFNDKINKFLKNNKEVNIIGIDRGE |
| | | | | | KHLLYYSVVDQQGNVISQDSFNTVNGVDYLAKLKQKEISRDEARKSWTIIENVKELKSGYLSHVI |
| | | | | | HKLAELIIEHNAIVVLEDLNIGFKRGRFKVERQIYQKFEKALIDKLNYLVFKDKTNRLQAGHYLN |
| | | | | | ALQLTNKFTSFQKLGKQSGILFYTTAAYTSITDPITGFVKNLYKNYASVKDSIAFWNSFESIHFNT |
| | | | | | QKDRFEFTYDTKKINSKNLNKDTKEDKEIQTKWMVCSCVIRSRYDKKSKTHELFDVNKKLKQL |
| | | | | | CEKFNIDYKNYNLKHEFSKIDKKDFHKSCLYYFNSILNLRVTDSSQASGTPENDFILSPVEPFFDSR |
| | | | | | KKYRKLPENGDANGAYNIARKGICMLNKIKQWDEQKRLDLLITKKDWQEYCQNNEIIKQQMN |
| | | | | | KMKHKA |
| MG29 | 6470 | MG29-147 effector | Protein | Unknown | MNENNSIWGNFINKYNLSKTIRFELKPVGKTIDFIKENGLIEEDKQREKDFNEVKKIMDEYYVEFI |
| effector | | | | | ENSLKNIKLDLSDLQEYYTIYFELKKDKYNSDLKKQFKNIQKKIANNMYQQIKDVDNFNNIFDEK |
| | | | | | FVNVVLPKWLKEKGREQDEMLLSKFKKWTTYFDGFFNNRQNVLSNDLIPTSIFYRIVVDNLPIFL |
| | | | | | DNIAKYDKLKKINGFPLESIEKNFAKYLNNVSLDYFFSLENFNNLLNQEGIDLFNLILGGYVENNS |
| | | | | | KICGLNESINLYSQKPENKETSKQLKQLIMMPLYKQILTEKKSFSEKFGIIQDNQELVNLIDDIYT |
| | | | | | NNYVLNFDNLQKLIKNLNEYDLNQIYVNTLSLQKISKSIFKDLFVIRNGLKEFIKNKEHIKSDKKS |
| | | | | | EKKLEEILNQKYFSIFEIQEGVKLLKLSKEGNSFSDNFMIDYFLENINDKLFENIKNNYNQFKSINV |
| | | | | | NHLTKITSEDIQTIKTLLDSLKELFDYIKPLYVNLNIGTNTKIQEAYNLDSLFYIEFNKIYNAFSVIIP |
| | | | | | TYNKVRNYVTKKQKNVKKFKLNFNCPVLLKGWDVTKEPENHSVLFRKDGDYYLGIMPKGHTH |
| | | | | | MFSNIENIDNDGEYYEKMVYKQISDASKDIQNLFVKDNKTQRIVGRKEKEGDNKGKNVELENAR |
| | | | | | KKYLPEDIQEIKKKKSYLRSSPSFNEADKNRFIDYYKERVIDYENYNMFNFKFKESEEYLDENDFI |
| | | | | | KDVDNQGYKIEFIKINEQFIMD |
| MG29 | 6471 | MG29-148 effector | Protein | Unknown | MPRTESIWDGFTNQYSLSKTLRFELKPIGKTQEYIKERGLITEDKEREKNFDVVKNMMDDYYRH |
| effector | | | | | FIEESLVNVAINTNHLKRYQEIYNSLKKDRTNQKLQKEFSQAQGYLRKQILKGINENPNFQHLFK |
| | | | | | QEFLTKILPTWLGDKDRTEDAKRVLEFNKWSTYFTGFFDNRKNVFSEKEIPTSVIYRIVNDNLPK |
| | | | | | FLDDISRFEELKKLNGFDYSEVESNLKQELEGKCLNDVFSLDNFNNCLNQSGIDLENLIIGGKSDE |
| | | | | | GGTHFKGLNNIINEFSQQQDNKAIRRLKLIPLFKQILSDRESFSFISEKFDNDKELMEELLDFYEKI |
| | | | | | RSIQIIDKLKDITLKLPEYDLERVYLKNDANLTQISQEIFDNWAYITSALQEYAVSVLGLKESKAK |
| | | | | | AWVQKQSYFSIYELEEAIRQKSKDAAKNPICTYLSRFDKGERNLFEKMENNSKPLFDITVSDDRK |
| | | | | | LLTRSREADMETIKAFLDSILGILHFVKPLHLEPKKKSEEGLQDAFETDPDFYLEFNAAYDALNKI |
| | | | | | SPLYDKVRNYITQKPFSTKKFKLNFQNSTLLAGWDKNKERDNWSVIFRKDGLYYLGIMSSEDNK |
| | | | | | IFSKTMPNLKDCSDYFEKMNYKLLPGPNKMLPKVIFSKKNIAFFNPSKEILSIRDHASYTKGGTPQ |
| | | | | | TDFDKKEFDLDDCHQMIDFYKTCLEKHPEWCEYGFRFKATDDYADISEFYNDIANQGYRIFFEKI |
| | | | | | DSDYITKLVDDGKLYLFKIWNKDFSKFSKGKKNLHTIYWEELFSEKNLSDVVYKLNGEAELFYR |
| | | | | | EASLEGCITHPKNEPIQNKDPIKGKKTSTFPYNLIKDKRYSKDKFLFHCPMTLNFKAGDKNWET |
| | | | | | NKKVNEHVAKNCDDICILGIDRGERNLASYVLIDSKGKIISQSSLNVTSDDFERTRDYQNKLDTLE |
| | | | | | GSRNEARKNWKKIANIKELKEGYLSQVVHQISNIAIDNNAIIALEDLNFGFKRGRFKIEKQVYEKF |
| | | | | | EKKLIDKLNYLVFKDRLEDEIGGALHAYQLTKKFESFKVLGKQSGILYYVPASYTSKIDPATGFV |
| | | | | | NLLYPRFENIEKAKAFFKKFDSIKFNSSKDYFEFSFRYSNFIEDMEKDAIIKDDWTVCSVGNRLVN |
| | | | | | KRLEKSRGYETIEKDLTEELKSLFSKNQIEFEEGNDLIEAISSQDNAEFFKNLIYLLKCTLQLRNSR |
| | | | | | TGTNEDYILSCVKDKNGRFFDSREANDNEPKDADANGAYNIALKGLMLVERLKNSDLESKKLDL |
| | | | | | RIDRNEFLNFVMKRAL |
| MG29 | 6472 | MG29-149 effector | Protein | Unknown | MDNSSTIWDDFTNMYSLSKTLRFELRPVGQTLEHIKEKGLIEEDKEREKEFNEVKKIIDEYYKYFI |
| effector | | | | | EESLREVNIERNDLEEYQGSYNKLKKPEGKYDENLKKKYSEMQTKLRKKIFSKIKEHENYKHLF |
| | | | | | GKELILSVLPEWLEKNNNLEKIDKVKRFSRWATYFKGFFDNRENVFSEKDISTSIIFRIVNDNLPK |
| | | | | | FLDDISRFQKLKEFEGFSYKEVEEGFSKELEGVDLESFFSLNNENNCLNQSGIERFNLLIGGRSNE |
| | | | | | NGMRTRGLNELINEFSPQRNENGKQIRRLKLMPLFKQILSDREKSSFIPEKFMNDKEVIDSMKEF |
| | | | | | YNDLDENIFNQIRNVVTQMHDKELKQIYFRNDRNLTDISNKMFDDWETIKNGLNEFAKLELGYT |
| | | | | | EKKAEEWIKKQKYFSVYEIEEGIKKLNIESVSSEHTLCDYFSKMNVDDKDIIEEIKTKYSVVNELSI |
| | | | | | PADKSLANESDEKNIEKIKDFLDSALILLHFLKPLYVESGRKEEEKLEQALELDTDFYHDFNQIYE |
| | | | | | KAKAVIPLHNKVRNYITQKSFSTNKFKLNFQNSTLIDGWDENKIKDNFTVILRKYSNPKKSYDYF |
| | | | | | LSIMTPEDKKIFDNTARSSSVFYERMIYRQIADASKDIQNLMVINNKTVCKKGRKDDDGINRRLE |
| | | | | | EIKNEHLPEEINEIRKKKSYLKSSENFSIEDKNKFIGYYIERAKSYWGFDFDFEDPSQYADENGFTS |
| | | | | | HLDSMGYNISFEMVDNNHINKLVNEGRLYLFQIWNKDFSEYSKGRPNLHTIYWRMLFDENNLK |
| | | | | | NVVFKLNGKAELFYREKSISRNVTHPKNKPIKNKDPLDDKKESKFEYDLIKDRRYTEDKFLFHCP |
| | | | | | ITINFKSSDKSWEINNQI |
| MG29 | 6473 | MG29-150 effector | Protein | Unknown | METSESIWDNFINKYSLEKTLRFELKPIGNTLQTIKEKGFIKEDKKREENFNKVKEIMDDYYKDFI |
| effector | | | | | EKCLSGIEIENLKDHEEIYKKLKQNYKDKYLKKEYSESEKRIRKSIYEQITKKDNFDKIFKEEFLK |
| | | | | | QILPDWLEKKGKEQEKELVLSFSKWVTYFTKFFSNRKNVFSEDEIPTSIIYRIVNDNLPKFLYDKS |
| | | | | | KFEELKDMGFDYSEIEKNFKNELNGKNLEEFFSLDNFKNCINQSGIDRFNLIIGGRSEADNVKIKG |
| | | | | | LNEIINEFAQKNKNSESDIRKLKLVPLFKQILSDSESPSFILERINNKKEMFEKIQNFYSSFNDISDK |
| | | | | | LISTIKNLNKFDVNQIYLKNDSYLNEISKNIFGDYDIIKEGLKLYFDDKSKNKKESEKAQKSEFIPIS |
| | | | | | YINEGIKKLELELNDNSSIVDYFSNLSEQPSNSDNNLIEAVKNKYKCLEPLKENIDNINQISEEGIVK |
| | | | | | IKNFLDSAMSLYHFLKPLYLNSNKNRSNELELLDIDLDFYNEFNKEFEELEKIVPLYNQVRNYITR |
| | | | | | KFITKKFKLNFDKSTLANGWDLNKEKDNLAVILRKRNPVTKKYDYYLGIMIKESTKIFKGIPEGR |
| | | | | | SVNKENSYEKMIYKFIPSPNKNLPRVFLSSKKGKDKFKPSNEIVKIYEEKSYIKDSEEKDKDKKKF |
| | | | | | NTKDLHKMIDFYKDCLKNYKKWSKYFNFNFKDTNEYHDISEFYTDVEQQGYKISWKYYDEEYV |
| | | | | | NNLIQEGKLLMFQIWNKDFSGYSKGKQNLHTIYWNLLFSEENINGLIKTYKLNGNAELFYREKSI |
| | | | | | EREITHSKNNPIKNKNPINEKKESVFKYDLIKDKRYTEDKFFFHCPITLNYGTKHNEPISKTVNKII |
| | | | | | QDSQSNINILGIDRGERNLLYYSLIDLNGKIIKQGSLNTISDDKKRELDYHSKLDQIEGRRDEARK |
| | | | | | NWKDIDNIKNMKEGYLSQVIHQIAKLAVEYNAIITLEDLNFGFKTSRFKIEKQVYQKFEKMLIDK |
| | | | | | FNYLAFKDRNEKEPGGMLKAYQLTDEFKSFKNLGKQSGIIYYVNAGYTSKICPKTGFVDMLRPK |
| | | | | | FENVKKSKEFFSKFKYIKYKKDEDLFEFNFEYSKFIVDKNKNPKLKRDNWSIWSNGIKLDTKRDN |
| | | | | | NGYWRTEKINVTEELKKLFNANEIDYTSENNLIEKIKESNDKNFHFSLIYLLRLILRLRNSYIDYEV |
| | | | | | EEIRKKNENKFNIRDYDYILSCVKDNNGEFFDSRKSSGNEVLDADANGAYHIALKGLML |
| MG29 | 6474 | MG29-151 effector | Protein | Unknown | YFTGFFDNRRNVFSKDEIHTSFIYRIVHDNLPKYIDNLERYDKLKKYSDFNCKQLVNDFSSELQGE |
| effector | | | | | DLSKFFTLEKFNECLSQPGIERFNLIIGGKSLEGNRKIPGLNEVINLYSQNLTNEENKRAVRKLFM |
| | | | | | LPLFKQILSDRTSASFILSAIEDDAEVISIVNELYSRISKDFELFQSTFKGLTKENTSQIYINKALLNE |
| | | | | | VSNQIYDDWSILENSLKCFAKEKLKLNTEKKIDEWLKKSVYFSLDEIKQGIEFLKSENVTWEKFI |
| | | | | | EHYNLLTYDNKPLIDAINENYNAVKGLDLKKNEKKLLTEQKEADVTKIKQFLDSVMNLYHFLKP |
| | | | | | FNMNQDFEKETIHVEVDTEFYKDENDIYSGFAEVIPVYNKVRNYVTQKTFSTNKFKLNFKNSTLL |
| | | | | | QGWDKNKESDNWSALFQKDGNYFLGIMSEGSGNNKLFKKKVYVANNVPTYRKIVYKLLQGPN |
| | | | | | KMLPKVFFSKKNIGKYNPSENILRIRNHSTYTKNGTPQDGYTKKEFSLADCHKMIDFYKDCIKK |
| | | | | | HKEWNEEFDFKFRKTSEYTDISEFYKDVADQGYKITYEEIPDKIINEYVDTGKLYLFQIWSKDFSP |
| | | | | | FSKGRKNLHTLYWQELFSPENLRDVRFKLNGEAEVFFRRKSIDPKITHPKNKPILNRNPIREKKE |
| | | | | | SNFSYDLIKNRRYTEDKFFFHCPVTLNFKSKGSDNSKSINSKINEIVKKNPDVNILSIDRGERHLLY |
| | | | | | CTLLNLKGEILKQKSFNMVFDDVQREHSYHDKLDTLEKERQAARKAWKTIHNIKELKSGYLSQ |
| | | | | | VVHQISKIAIEDNAVILEDLNEGEKKGRFKIEKQVY |
| MG29 | 6475 | MG29-152 effector | Protein | Unknown | MKTCQSLQIISTFIKKIKDEGIDLTEIETNFNNVDLKSIFSIEYFSKTLTQSGIDFYNELLGGKTESE |
| effector | | | | | GKKIKGINELVNLYNQKQNDRSKKIPFLKPLYKLPLFERTSVSFRYEPVKDDQDLLNRIHSFYYN |
| | | | | | DLRQFYDEDDGQTKDVLDELKKLLENIHNYREGLYVNGGLTLTQISQKIFGRWNYINEALSDYY |
| | | | | | DTVINPPKPDKNGRSIVRTKKEEKEKERWLKQKQFLIITIEEALSQFKQKETNEELKNKITETTLC |
| | | | | | DFFKRCGANEEGKDNLFERIEQNLKMKNEHGESVKDLLHTNSKKSLLEDKSSTLLIKNFLDVLQ |
| | | | | | GDKEDITSGLLHFIKALIPREEVGLKNEDFYSQFEKYYRQLSEITPLYNKARNYLTQKPYSIEKVK |
| | | | | | LNFENSTLLDGWDRNKEKDNTCVLLRKKMNDQYFYFLGILNKKHKKIFENYPTADKEPYYEKM |
| | | | | | VYKLVANPTKDLPNLVVIDGKTSMVKGRKNHEGENKVREQKLNKYLPPVINRIRKSKTYSSNNL |
| | | | | | VKDDLTKFIQYYQERVREYFTDLNFHFKEASEYNSWADFLDEVKQQGYSINFKTISEDYINNLVN |
| | | | | | EGKLYLFKIHNKDFSENKKSKGKDNLHTLYWKMLFDERNLKDVVLQLNGKAEVFYRPKSVNYS |
| | | | | | EDIWQKGYHYDELKEKFNYPIIKDKRYAEHKFFFHVPITLNFKSVGKNNINEKVNQWLMNNPNI |
| | | | | | HIIGIDRGERHLLYVSVINQKGEIVEQCSLNEISEYKGHSFSKNYHQLLDKREGEREKARKDWQ |
| | | | | | TIENIKELKEGYLSHVIHKITQLILKYNAIVVMEDLNVGFKRGRQKVEKQVYQNFEKMLIEKLNF |
| | | | | | LAIKDKKPEEPGGVLKAFQLSNKFESFKKLGKQSGIIFYVPAGYTSSIDPLTGYVHYLTPLKLADS |
| | | | | | IEKARNFYKKFKSIRFNTVNEWFEFSFDYNDFERVRYEGKSDWVICTSNSERYVWNKALNNGKG |
| | | | | | GAEQVKVTERLEVLFGEYEIDYGSGKCIIDQIVNVNDLDKERTAKKFYSTLNFLLNTTLKLRHNN |
| | | | | | GMKGKEEQDYILSPVSPFFDSRKENAKSASEQKLPTDADANGAYHIALKGLLLMKRLKEIGVEA |
| | | | | | FEKSKNSKDGKSQWLPNDEWLVFVQNKNSVSEPV |
| MG29 | 6476 | MG29-153 effector | Protein | Unknown | MNSIFDRFTQQYPLSKTLRFELKPVGKTAKLISEFNDDFPESTIAKDEEKSKAYPFAKKILDDYYR |
| effector | | | | | HFISEVLSKSELDATKIKQAFICYKECQSKNATADADAKKSEYKIQKSILRKVIADFFSSEGLELSA |
| | | | | | MFKGAKKNCAIYEYGELFKEASPLFVWLQNRLVNEVITQEEFDTQTGIINKFNGFSTYFTKYKV |
| | | | | | NRENLFANEEKASSIAYRVVDENMEKFFDNCITYQKIMQKYPNEEFAAELKKCEQFFTPENFGIC |
| | | | | | LTQSGIDVYNQIIGKKSDDTYGKGINQQINEFRQKNALRRNDAPLMTVLFKQLMSESERVFVIETI |
| | | | | | DSDEELFSVVKDAYNTCIALVEGLSILCDSSLTDENLSDIFIRPDGLSNLSQKVFGKWDIIDSALNM |
| | | | | | KKESIGQKKFDAKYSKVISLLDLQTMCDAYISVIDDSEIRRNCTFSDYFKTFESSLIKSAYLEAEEV |
| | | | | | LNSIGLDKDKSMPTNDTDLGGKGFRQLQKIKQLLDSINEAVHFYKPFLLEKEGKAIEAEENNKEF |
| | | | | | YNEFLLNYRDLSVFPKIYDKVRNYATKKPYSKDKFLLNFDKPTLLDGWDVNKEESNLALLFIKD |
| | | | | | GKYYLGIMKNPRLFSNLPVKGIANENEPAYEKVIYKQVSGACKMFPKVFFADLNRELYKPGSEI |
| | | | | | QRIREEKSHLKGGSEDSKNKWIQFCIDCIDKQPEWKTYFKFNFKEPQEYPDVNSFYKDADAQM |
| MG29 | 6477 | MG29-154 effector | Protein | Unknown | MSLAAFTNQYQLSKTLRFELIPQGRTLQHIQQKGLLSRDQQRADSYQQMKKTIDAFHKHFIDLA |
| effector | | | | | MQQVHLSKLAEYQDLYEASGERKKTDEYKKELEKIQAELRKEIVAGFKAEGVSEIFGKLDKKEL |
| | | | | | ITDLLEKWIREQNDKDIYFDAGFKNFTTYFGGFHENRKNMYTDKAQSTAIAYRLIHENLPKFLDN |
| | | | | | CRIFDSLKARPELAEKLPLLYTEIAEYLNVASIDEAFALDYFNEVLTQKQIDVYNLIIGGRTPEEGK |
| | | | | | KKVQGLNEYINLYNQQQKDKNNRIPKLKILYKQILSDRESTSFMADAFESDADVLDAINAYYHNE |
| | | | | | LISFFSADSEEAIHVLHEIRSLLKHIASYDTERIHVRNDTQLTHISQKLFGSYAVLGDALSYYHDVV |
| | | | | | LAPSHQEDYQKAKTESKRGKLEKEKEKFTKQPYISLALLQKALDYYVLSLDDTHEVRKRYSTNC |
| | | | | | ISDYFHIHFTAQKRADSDKEFDLIANIEAKYSCVKGVLEHYPSDRKLHQDKKTIDDIKLFLDSLME |
| | | | | | LLHFVKPLVLPPDSTLEKDNVFYGQLEPLYTQLELIIPLYNKVRNYATQKPYSTAKFKLNFENAQ |
| | | | | | LLNGWDENKESDYLTSIMRKDGNYYLAIMDKQHNKVLKKAPKAEYGRGAYEKMVYKLLPGVN |
| | | | | | KMLPKVFFSKKNIDYFAPSTQLLENYKKETHKKGENFSLHDCHTLIDFFKASIEKHEDWRNFGF |
| | | | | | QFSETATYDDLSDFYREVSHQGYKVTFQPIAENYIHELVDEGKLYLFQIYNKDFSTYSKGRPNLH |
| | | | | | TLYWKALFDPDNLQDVVYKLNGQAELFYRTASIRAEDRVIHTRGQAIDNKNPLTIKQQSTFEYD |
| | | | | | LIKDKRYTVDKFQFHVPITMNFKAGGRDNINQEVLAFLKNNPHVNIIGIDRGERHLIYLTLIDQQ |
| | | | | | GNILKQETLNTIVSERYPNETPYHTLLATKEKERDAARKNWGTIENIKELKEGYLSQVVHKIAK |
| | | | | | MMVEHNAIVVMEDLNFGFKRGRFKVEKQVYQKLEKMLIDKLNYLVFKDKDAHEPGGLYKALQ |
| | | | | | LTSKFISFKDLGKQSGFLFYVPAWNTSKIDPTTGFVNLFDTRYESIGKAQDFFGKFDTIHFNGAKG |
| | | | | | YFEFVFDYDAFTTRAEGTRTKWTVCTYGSRIMTFRNPDANNQWDNKEIDVTQEMEDLFGTYGI |
| | | | | | TYGDGSDIRGELLRQTDKGFYERLLHLFKLTLQMRNSKTGTDIDYLISPVMNARGEFYDSRRAD |
| | | | | | STQPKDADANGAYHIAKKGLWLLEQINQADDLKKVKLAVSNKEWLRFVQK |
| MG29 | 6478 | MG29-155 effector | Protein | Unknown | MKTFIDKFTGLYPVSKTLRFEARPVPATKEWLESDDCHVLENDITRDEYYPVLKQLLDDYYRYYI |
| effector | | | | | EEALAGFRLDEDLVHSAFIFYTEKDFEKLESGLEKLRKNLVKVFDSRKDYLLGSGQLKDLIRLNF |
| | | | | | KKKKEVLDSHLVSWIKDNPQYTAKQREDFLAAIGSFEGFVTYLGDYKQTRDSLFTDEKIASSIAY |
| | | | | | RVIHENMLLYFSNIRIYERIKETYPDLYKQINKFEEFFIPESFSGILSQSQIDRYNYNCIGRPVDDID |
| | | | | | YKGVNILINEYRQKMDISSNELPCMAVLYKQILSDRESFMPESIKNEAEAIEIARKGYDVAFDNLC |
| | | | | | NLEELFKQNIKNNNHDNIYIKCSAINDFSKIVLGDWKILGETLKNAKYKGDVISLGQLLELNSFPI |
| | | | | | LEFFNDTLESVIGSLSVEKVNELKSDLDKMLSEIQKYKPFFLFKGTKPLDVPDNGISFANEFKTIY |
| | | | | | EQLTEFIGVYNRIRNFATKKPYSMDKIKLNFDNPSLLAGWDLNRESTNGSYLFLKDGKYYLGIAN |
| | | | | | NESRDLFTDIRKKNVASHDTNDVYNKVEYKQISGAAKMLSKVFFADKNVELYGHLLTDRIIDIKA |
| | | | | | NKLYTTAANDKAALAEWIDFLKAAIAIHPEWNNYFNFKFRDSSEYANINEFYSDVDNQAYTLTTI |
| | | | | | PVSAEYIDELVDSHKMFLFQLYNKDFSEHSKGKENLHTMYWKGIFSKENLEAINDGTMPFIKLN |
| | | | | | GEAEMFMREASIPRKITHPREVPIATKNPVYKPMDSVYHYDIIKDKRFTERKFFFHCPITLNYRAA |
| | | | | | IKGNFNVKINEFVTGNPDINIIGIDRGERNLLYYTVINQKGNILAQGSLNRIGHKYTAKNKDFTAIT |
| | | | | | DYRLLLDIKEQERNNARQAWGVIKNIKDLKAGYLSQAVHEICQLMLQYNAVVILENLDIGFKRS |
| | | | | | RAKVEKQIYQKFEKAMIDKLNYLVFKDRGYHENGSFGKGLQLSAPFESFSRIGKQTGCIYYVSPS |
| | | | | | YTSQIDPKTGFVNLLGSKLKYESIAKAQDILKRFDSISYNASNEYFEFAFDYKNFGVEMEKTNWV |
| | | | | | VCTCGSRRPEYSPEEKKIKNYHVTTELKALFDSQGIEYADGKDILSAIVSVKDKSFLEGVLFHLRL |
| | | | | | ALKMRYIGEAPEDDFFLSPVEFAPGNFFDTREASQNEPQTADANDAYHIALKGLMTIQSIKDGKL |
| | | | | | AKYKVGGEKAAWFKYMQSQEFRKA |
| MG29 | 6479 | MG29-156 effector | Protein | Unknown | MSAQSALSTLINKYSLSKTLRFELIPIGKTKESIDRKGLLSQDVKRAQSYKEVKKIIDEYHKEFIEK |
| effector | | | | | SLINAKLKGLEEFSKLYYKLQKEDKDKKNIKKMQDNLREQISDLFKNNKKDKWNILFKEDLIKK |
| | | | | | ELPLFAKDDKQKNLINEFNKFTTYFTGFHKNRKNMYAEEEKSTSIPYRIIHQNLPKFLDNIRIFEKI |
| | | | | | KKNKINTDVIEKELSLFLNGIKINDIFSINFFNDVLNQKGITFYNTILGGVSEKDRTKIKGINEYVNT |
| | | | | | EYNQKQLDKKSKIPKLKQLYKQILSDTETASFVLEQFENDNQLLEKIEQFYNTELINYETEGKTQ |
| | | | | | SVFLQFEQLFKNMQNYDASKIYISNLSIANISKIIFGDWSIICNALAEWYDKHNTKGKKINEYKKE |
| | | | | | NFLKQDFSIQQIEDAVLEYKNDTLNKEINFLLNYFASFLNEKSKKNIIQRIETEYSKVKDLLNTDYP |
| | | | | | EKKKLASDKDNVSKIKAFLDSLMDFLHFVKPFNIKKDTGLEKEENFYSIYVPLFEQIDKIIPLYNK |
| | | | | | VRNYLTKKPYSTEKIKLNFENSTLLDGWDLNKESDNTSVVLRKDDLYYLGIMDKKHNRIFKELP |
| | | | | | SQNGNESSYEKMIYKLLPGPNKMLPKVFFSKKGKKQFKPSKKLLKKYEDGTHLKGDNFNINDC |
| | | | | | HNLIDFFKESIAEHEDWKQFDFKFSSTSSYKDLSNFYKEVEKQGYKITFQNISENYINQLIDEGKL |
| | | | | | YLFQIYNKDFSKYSKGTPNLHTLYWKMLFDNDNLKNIVYKLNGKAEVFYRKSSLILGDNIVHKA |
| | | | | | GEAIINKNPDNEKKHSTFDYDLIKDKRFTLDKFQFHVPITLNFKSEGRQNLNEDVRKFLKNNPDI |
| | | | | | NIIGIDRGERHLLYLTLINQKGKILFQKSLNEITNEYNNKNGKSQIKSTNYHSLLDKKEKKRDEAR |
| | | | | | KNWGIIENIKELKEGYMSQIVHYISKLMIEKNAILSLEDLNFGFKRGRQKVEKQVYQKFEKMMI |
| | | | | | DKLNYLVFKDKKANETGGLLNALQLTNKFESFAKLYNQSGFIFYVPAWNTSKIDPITGFVNLLKP |
| | | | | | YYENLNKSQEFFKKFNNIKYNPKQEYFEFNFDYKNFTNKAEGSKNVWEICTTNNERFMWDKTL |
| | | | | | NSGKGAQKAVDVTQELKKLFDSSKINYLNGNDIKEDIINQNSADFFRKLMKLLSVVLSLRHNNGL |
| | | | | | KGKDEKDFILSPVEPFFNSLNAKMEEPKDADANGAYNIALKGLLILKQINESEDLRKIKENLSNKE |
| | | | | | WLKFAQSKSF |
| MG29 | 6480 | MG29-157 effector | Protein | Unknown | MLSNFTNQYQLSKTLRFELKPIGNTLEHIEQKGLLSQDEQRAENYTVIKEVIDTYHKAFIEESLAS |
| effector | | | | | VVFDNLERFEELYLKSNKDEKEQKEFEKLQENLRKEIVKNFKVHPKWNNLFKKELIKEDLLAFE |
| | | | | | QITDEQKEVVKEFTNFTTYFTGFHENRANMYTDKEQHSSIAYRIVHDNLPTFVNNKKAFESILQK |
| | | | | | YPQLISDAKSSIEEELLGAVFEDMFLLQYFNHLPSQTHIDLYNTMLGGVKRDDLKIQGFNEKINL |
| | | | | | YRQANGLNKKELPNLKPLYKQILSDKDTLSWLPEAFETQEELVGAVESFYQEKILAFECCDGRV |
| | | | | | NLLEKFKEIFSQTQLYDTSKIFIKSDKPLTDISQALFKNYGLLKEALWQKHLDDNPKLQKSKKIE |
| | | | | | ESEEKFFKQKYFTLSSLQEAIEFAKLSANVWNYFQENLDTYIKQIEENHTIWETDKTNTATTKSF |
| | | | | | LDSLINLQRFLKPLNVQTDSDKDIAFYSTFDSYFEALTQIVKLYDKVRNFKTKKPYSLEKFKLNFE |
| | | | | | NSTLLDGWDVNKEPDNTSILLRKNNLYYLAIMDKKYNKLFCNLEKSTQSDVYEKIEYKLLPGAN |
| | | | | | KMLPKVFFSNKNIDYYNPSKKLLENYKDGIHKKGDNFDIDFCHELIDFFKVSIQKHEDWKHFKF |
| | | | | | NFSPTKSYEDLSGFYREVEQQGYKISYKNIDTKLVESWVNDGKLYLFQIYNKDFSPYSKGTPNMH |
| | | | | | TLYWKALFDEQNLANVVYKLNGQAEIFYRKKSIEYTEDKLKKGHHHEELKDKFAYPIIKDRRFA |
| | | | | | FDKFQFHVPITLNFKAEGNENLNQKTIEYIKINDIKIIGIDRGERHLLYLSLIDLNGRIVEQYSLNQ |
| | | | | | IINSYNGKEHTIDYHEKLAKKEDERALAREEWGVIENIKELKEGYMSHVIHRITTLIVEHNAIVVL |
| | | | | | EDLNFGFKQGRFKVEKQVYQKFEKALIDKLNYLVDKKKTPSDLGGVLNALQLTNKFVSFEKMG |
| | | | | | KQNGFLFYVPAWNTSKIDPVTGFVNLFDTRYSSVEKAKEFFGKFKSIRYNSVKEYFEFEFDYNDF |
| | | | | | HNKALDTQTQWTICTYGERIKTFRNKDKNSQWDNETIHLTTAFKNHFGNYQGELKEYILAQDK |
| | | | | | KEFFEQLLDLFKLTLQMRNSITNSEVDYLISPVADKNGNFYDSRKADSSLPKDADANGAYNIARK |
| | | | | | GLMLVERIKESTDVKKVDFKLTNKEWLQFAQRG |
| MG29 | 6481 | MG29-158 effector | Protein | Unknown | METNFFKSFTKQYSLSKTLRFELKPIGKTIANIETKGLLAQDNQRAESYKKVKKTIDEYHKYFIE |
| effector | | | | | LALKAVKLTKLDEYYELYSKNKEDRDDAAFKKVKEDLRKEIVAVFTKGGFKEMFIRLFSKELIK |
| | | | | | EDIAAWISEHPKFAEDLKFVKEFDNFTTYFTGFNENRKNIYSAEDKSTAIAFRIVHENLPKFIENIK |
| | | | | | LFKTIKEKHANLDFSPILKEMEEVIQGVTLDEIFTLDYFNHVLSQNGIEFINFIIGGKTLKNGDKIK |
| | | | | | GLNEYINLYNQQQKDKNKRAPKFKQLYKQILSDRTSISMRFEEFENDSDLLETVEVFYQSELCEY |
| | | | | | ETEGKTHNIFEEIKTLCESIGTYNLEKIYLRNDSNLTNISQRLFGSFSVFRDSISFYYDTVVDTNFQT |
| | | | | | KYANAKTENQIKHLNDTKNKWNSDFISLGLLQKTLAKYIETLDADSEIRKIYTPTIITDYFKRHIIK |
| | | | | | KEVEEAKNNETIKKTTDVELFYSITGQYLGVKGLLNIEKTDNKTLAQEKEKVHQLKSFLDSILEL |
| | | | | | NHFVKPLFLTDDSISDKDDAFYSQFAPLYEQLNKLIPLYNMVRNYLTQKLYCTDKIKLNFENSTL |
| | | | | | LDGWDVNKEPDNTSVILRKDGLYYLVIMDKAGKKVFMDVPKIAYSGTFYEKMNYKLLPLVNQQ |
| | | | | | LPRVFFAKSRIEFFKPSEAIQENYKKETHKKGDTFNIKDCHALIDFFKASLAKHEDWKHFNFKFS |
| | | | | | PTKSYQDLSGFYREVEHQGYKMSFENIPTDYIDKMIEEGKIYLFQIYNKDFSVFSKGLPNMHTLY |
| | | | | | WKALFDENNLADVVYKLNGQAEVFFRKSSIEEKNKVIHKAHELLKSKNPNTPNNNNTFDYDLIK |
| | | | | | DRRYTVDKFQFHVPININFKASGSEIINAQTNDFLKNNRDVKIIGLDRGERNLIYLTLIDQKGNIIQ |
| | | | | | ESLNTISNKERKIETPYHTLLNIKEKERDAARKSWNTIENIKELKEGYISQVVHKIAEMMVKHHA |
| | | | | | IVVMEDLNFGFKRGRFKVEKQVYQKLEKMLIDKLNYLVFKDAQPTQPGGLLNALQLTNKFESF |
| | | | | | KKMGKQSGFLYYVPAWNTSKIDPATGFVDFLKPKYENVEKAKAFFSKFDSIKYNTVKDYFEFAF |
| | | | | | DYK |
| MG29 | 6482 | MG29-159 effector | Protein | Unknown | MSIMVENIYKNFNNIYSVSKTLRFELKPQGNTLENIRKINLIEEDEQRFKDFQEVKKIIDKYFKYFI |
| effector | | | | | EKNLDGAKLEDHQIKQFVDSYDKLKKNKNDDKLKKEFISYQDELRKSLHEQIKGKGFYKYFFK |
| | | | | | KDFIKEVLVKYLQNKGENENVYLVNKFNDRTTYFTGFNKNRENIFSTKEIPTSIYYRIVNDNLPKY |
| | | | | | IENIKNYEKLKEYFKDGLDLSQLEKDFEKELNGMSLDLFFSYYNFNSFLNQKGIDKFNEIVGGKT |
| | | | | | LENGKKIQGLNEYINLYSQEKKEKSIRKLKFVPLFKQILSDRETSSFILDTFENEQDILESVDNFYN |
| | | | | | KILNEKVEKEINDLFSNLEKYDRTQLFIKKDKTITNLSQKIFGDWSIIENALKKYALNKINVKEKD |
| | | | | | LDKWYKKNKYFSIYEIEEGIELLNIVNDMDKSVYDIFVRKVNDKPKYSICGFYSLFKPEENSLFEE |
| | | | | | IKINYGEFEKLLNNGFNKEIEENTIIIKKFLDSIMNLYHFINPLYLNQKKDSEDNEPVAFEFDSEFYS |
| | | | | | DYNKIIETLSEHIPLYNKTRNFITKKNFSTSKFKLNFQNSTLLDGWDINKERDNWAVLFRKKGNYY |
| | | | | | LGIMSKGNNKVFTNVTESDSNEFFEKIEYKQISDASKDIQNLMIIDGKTVSKKGRKEKGGYNDGK |
| | | | | | NLILEELKNKYLPPEINEIRIKKSYLKSNSNFSEDDKNKFIDYYIQRLDYWNFDFNLKKSSEYVDFN |
| | | | | | EFTSHISNQGYQIKFKKISDDYINKLVDEGKLFLFQIYNKDFSKFHKGRKNMHTLYWEELFSEDN |
| | | | | | LNDVVYKLNGQAEIFFRKASLKDPVIHPKNQDIKNKDPLNNKKTSNFKYDLIKDRRYTVDKFLFH |
| | | | | | CPITMNFKAKGSSYNLNYMINEKIKENRYNFKILSIDRGERHLAYYTLINSKGEIEKQGTYNLVVD |
| | | | | | DENRERNYHQKLDKLEGERDQARKSWKQITNIKELKEGYLSQIVHQITKLAIEENALIVFEDLNT |
| | | | | | GFKRGRFKIEKQVYQKFEKMLIEKLNYLVFKDKNNNELGGLLKAYQLTPKFESFQKMGKQTGI |
| | | | | | IYYVNADYTSKIDFSTGFVNLLHPKYENIIKSKEFLNKFQLIKYNSKEDLFEFNFNYSDFGYPNFKK |
| | | | | | DNWSIWSNGNKIINFRNPEKNNQFDTKKFNPTDELKNLFDEFSIKYDGGENIISEILKIDDIKLFKT |
| | | | | | LTYILKIILQLRNSSNKTGDDYILSCVKDKDGKFFDSNNKNPIEPKDADANGAYHIGVKGLILMDR |
| | | | | | IKDAQDLKKVDKAIRRDDFINYHIERRWKTI |
| MG29 | 6483 | MG29-160 effector | Protein | Unknown | MEKNIFEDFTNLYSLSKTLRFELIPQGKTLEHIERNGLLFQDEERAKEYQNAKKIIDKYHQHFIEE |
| effector | | | | | VLKSVEIEKSLLEEYETLYFLMKKGEEEKILKELENIKSKIQKNISKQIEKHPRKKNLFSKELIDG |
| | | | | | GKKEEADIIEWLFSCKENNLTLFEDTEIGNVENAIEIIKQFKGWTTYFGGFHTNRENVYSDKDIPT |
| | | | | | SIIFRIVHDNLPKFLEDRDKFKQIQMNYPNLLQQKEIEENFMAELIFENKDCSTKLFSIVEVFQLEN |
| | | | | | FNLFLNQSGIDKYNTLIGGKFIEGEVQKRKGINEYINLYSQQQNEKQVIKSIKKLKMVPLFKQILS |
| | | | | | DRSSNSFVLDKFSNDGQVVSAIDYYFQTLEEELEKHSFSEIIELLFSSDHFMDYDFSKLYIKNGQGL |
| | | | | | SALSQVVFGNYSVIGAALREFALNSVNPKDRVRLSKSEESKLEKLMKTSYFSLKSIQVALMMYVE |
| | | | | | GEYKFSKIVEYFCEMPKKYTQLMGQYMENRKLFENYFLSVEKKLLKTESQDVESIKALLDSCQQ |
| | | | | | FLSSIDVLSLAKGNMVMEKDEHFYYFFEPFFLRIKEIIPLYNKVRNYITQKPYSVEKYKLNFENSL |
| | | | | | LAKGWDRNKEESNTALLFKKENNFYLGIMKKSSNKIFNDTEMIKNQGEGYQKVTYKLLPGANK |
| | | | | | MLPKVFFSEKSIGYYSPSEEILNIRNHSSHTKNGTPQVGYEKNEFSLTDCKKIIDFFKGSIEKHPDW |
| | | | | | KEFNFRFSETEKYQSIDEFYREVENQGYKITFNNISENYIHQLVEEGKLFLFQLYNKDFSKFSKGK |
| | | | | | PNLHTLYWKALFDDENLKDVVYKLNGEAELFYRKASLDKIITHPKNLPIINKNTKTVKKESLFEY |
| | | | | | DLIKNRRFTENKFFFHCPITMNFKSNGKNKFNDFTNEILKKNIDDIHILSIDRGERHLAYYTLLDG |
| | | | | | KGNIVKQNTFNIISNEKTTVDYHEKLEGLEVERDKARKEWKKIENIKEMKEGYLSQVIHEIAKM |
| | | | | | VVEYNAIVVFEDLNAGFKRGRFKVEKQVYQKLEKMLIEKLNYLVLKDKQPSELGGVFNAYQFT |
| | | | | | DKFTTFKDMRNQTGIIYYVRADFTSKICPVTGFVNLLYPKYETVEKSQEFFGLFDEIRYNKEKKY |
| | | | | | FEFKFSYKEFGKECPGDWILCTHGTRLENFRNSEKNSSWDTREINITQLFENILKSYGIEYENGTC |
| | | | | | LKNSIIVQKEKKFFVDLVRVLNLTLQMRNSKTGTQIDYLISPIADSCGNFFDSRNSDCRLPENADA |
| | | | | | NGAYHIGIKGLMIMERIKQLSEEKKLTLGIKNSEYFEFVQRRLSE |
| MG29 | 6484 | MG29-161 effector | Protein | Unknown | RKFEDDAEMIQANRQYFEEVLGNNTLFETGETPTEAMNQLFLSIENYDLSKIFIESPLLVTSISQKI |
| effector | | | | | YGSYAVIPQALEYYHDNHVNPSYAAKFNKAKSDKSRETMEKAKAAWVKGIHAVSVIHQAVIAYN |
| | | | | | DVLPDDAKLTDTQPVISYYKDIQYSEKTGESQQIFDALMRRYHQAKGMLNTDYPKGSKQILNNK |
| | | | | | SSFAIVKNLLDVSKAYVNAARDLTIKKPEGLDLDPLFYERLAKTYTYLQDLHALYDTTRNYVTQ |
| | | | | | KPFSTDKIKLNFDCAQLLAGWDFNVIDAKRGVFLVKNGRYYLVIIDNKHKKAMNNLPAPITNNC |
| | | | | | YDKYNMRLSKDAHMALPKKLFTKDNLKIPAIAEMERRCRDKNGGHHLRKSPDFDKDFMHQMI |
| | | | | | DTFKDIIKKDKDFDVFGFQFKPTHQYEDINEFYADFNEQALVTWYDKVDSDVIDSLVAEGKIYLF |
| | | | | | EVYSKDFSDKSTGTPNQQSLILQYLFSQDNLAKHHFKLNGEAEVFYRKASIDKDKAVVHKKGSL |
| | | | | | LENKNPARPNSKIAKFDIVKDRHYTEDKLFLHIPITLNNNAADMKSYAMNSKVLNTLKTNGGVN |
| | | | | | VIGIDRGERNLLNITVIDSAGEILHQESLNKIASGQDMVTDYHELLDKKEQSRAESRLNWQEVESI |
| | | | | | KEIKQGYLSQVVYRLSQLMLQYEAIVVLEDLNIGFKRGRFKIEKQVYQNFEKALINKLNYLVLK |
| | | | | | QLEATEVGGTAHGYQLTAPFESFQKLGKQSGWLFYVPAWNTSHIDPTTGFVNLHHFKYESVAQ |
| | | | | | ATDIIDKLSNIRYNPEKDYFEFAIDYNEFTFKGGDSQKYWVVCSTPYKRYVFDKKANMGRGGTK |
| | | | | | AIDINAELKALFAAHGVDYASGEDLRPQIKAKANKELLSQLLFLLKTLTAMRYTNASSYEDYILS |
| | | | | | PVANKAGEFFDSRKGDAALPLDADSNGSYHIALKGLCLLQRVYDWRGEEFKWLDLFISNNDWL |
| | | | | | KFA |
| MG29 | 6485 | MG29-162 effector | Protein | Unknown | MEPLDYNNFTNLYSLSKTLRFELKPIGETADYIEDFKSQNLKDFVTEDQQRAEDYKAIKELIDDY |
| effector | | | | | HRAYIEDKLSSPVDPKTGEMWINPQDLEYAYSHYENLKQDQKDPKNRKLWEYTQTALRKKLVK |
| | | | | | SFAGNSDLFKKELINRDLPNWLEQKGKWEEHKATIESFKKFTTYFSGFHENRKNMYSNEEQPTA |
| | | | | | IAYRLINENLPRFFNNYIQYKKMVDEYKDLKLTATPALLEKMRVESLDEIFKPRYFINLFTQTGID |
| | | | | | NYQQLLGGKTEETGEKEQGLNEQVNLFKQQIQKQAKEHAKKSGEKATKIYDLSGFTGLYKQIL |
| | | | | | SDRETTSFIPEAFDDDKRLLITLAEHIENVTKNNGLLNKLEEAIAQLASTDLQRVYIKSVGLTAISQ |
| | | | | | NLFHRYQIINLALKYYAKKELSAKDCEIYLGQKAFSIAELDEKLISYIQSLEDNDPLHEQLKKLTM |
| | | | | | PQHPLQGYLLEAVKLAQTKQDNRPELNDAISEVKQLLSLDELSKNRKTPVKEGENGGVGFRQV |
| | | | | | QKIKGMLDGFMAVSDAVKPLHLVDGRNHIDMPDMDAGFYGGFSEAYNNYTEATITLYNKTRNH |
| | | | | | LTKKPFSTDKIKVNFGTPTLLNGWDVNKETDNSSILFEKDGLYYLGIMHPKHKHLFNYTKGIDD |
| | | | | | AGNDKKLKTKDTLNKKVTETDIGGYRKIVYKLLPGANKMLPKVFFSNKRIDYFSPSSEVRKIRNT |
| | | | | | ASHSINGTPRPGFNKEDFSLEDCHTMIDFFKVSIKKHPEWTEFNFQFSDTNSYEDLSGFYREVESQ |
| | | | | | GYRMDFHKIKESYINECIEAGKLFLFQIYNKDFSPYSKGNPNLHTLYWKGLFEAENLKDVVLKL |
| | | | | | NGEAEIFYRKHSIKRKDIITHTANEAIANKNSENPKKESTFAYDIVKDKRYTKDKFQLHVPITLNF |
| | | | | | KAQSVSRFNDHVNKALQGNSGTHIIGIDRGERHLLYYTVINDKGEHIEQNTLNSISTDQGYAVDYQ |
| | | | | | QKLHTKEKQRDVARKSWGSVEDIKELKEGYLSHVVHKLAELIIKYNAIICLEDLNFGFKRGRFH |
| | | | | | VEKQVYQKFEKALIVKLNYLVFKQNEATKPGGYLNAYQLTAPFISFDKLGKQSGILFYVQAAYT |
| | | | | | SKIDPATGFINFLYTQYQSLAKSKQFFEAFESINFNSNKNYFEFSFDYNNFNVRQNLKDYQTKWIA |
| | | | | | CTHGDVRYSNKRNQQGQWETISINVTEQLKALFNEASISYQNGQDLKQALASAKNTRFYKSLYW |
| | | | | | LIKLTLSLRHSVTGTDEDFILSPIADKNGVFFDSRNATKQQPKDADANGAYNIALKGLWNLQQIK |
| | | | | | QWDGEENLKLAMKNEDWFKFINDWHNK |
| MG29 | 6486 | MG29-163 effector | Protein | Unknown | TAEAERGEAADPLKPLLVLDQLNKKRIPPEGDGEEGSEGFEQVRRIHECLDAHMSLQQVLRPLH |
| effector | | | | | LVLGRKPIDVASKDLGFYARFDEAIEDYNAMTIALYNKTRNHLTKKSFSTDKVKINFESPTLLAG |
| | | | | | WDLNKETANKSIILRQNGKYYLGIMHPRHPKIFSKPPEAKVGDEAYEKVNYKLLTGANKMLPK |
| | | | | | VFFSKKGLETHNPSEQILALYKNGEHKKGDTFNIESCHKLIDFFKSRIPLYKRDPSDPYGWEIFDF |
| | | | | | KFSPTKTYKDLSGFYREVEEQGYKLWFTHVTKAYIDEQIEQGHLFLFEIYNKDFSPFSKGKPNLH |
| | | | | | TLYWKGLFEQNNLDDVVLKLNGEAEIFYRKHSIAANEQIVHSANKAIVNKNENNPKPESTFEYDL |
| | | | | | VKDRRYTKDKFFFHVPITLNHKAQKPVRFNDQVNRALQKADDVHVIGIDRGERHLLYYTVVNQ |
| | | | | | KGEITEQDTLNTISTDQDYVVDYHHKLDQQERTRDKARKAWTNIDNIKELKAGYLSHVVHKLAE |
| | | | | | LIVKHNAVVCLEDLNFGFKRGRFKVEKQVYQKFERALIEKLNYLVFKDTTEGQPGHYLKAYQL |
| | | | | | TAPFESFKLLGKQSGILFYVGASYTSKIDPATGFINFLKPHYESLAKSKTFFESMDSICFNAKRGYF |
| | | | | | EFSFDYANFSVPQTLDDYQTAWTVCTHGETRYHNQRNDKGIWETKAVNVTEKLKVLLNEAGVS |
| | | | | | YQNGEELKDAIAAVKSSKFYRSLYFLLRLTLSLRHSVTGTEEDFILSPVADEGGNFYDSRNASDAE |
| | | | | | PKDADANGAYHIALKGLWNLEKIDQWDGESRLNLAMKNVEWFQFASEKPFKE |
| MG29 | 6487 | MG29-164 effector | Protein | Unknown | MVAYFKKHDTFKILDKKELIQKELDKWIKKYHPDLYFDKDFYNFTTYFTGYNENRMNMYSEEE |
| effector | | | | | KATAVSHRLIDENLPKFIDNIRAFQLVKDSPVAENFAVICADMEPWVNVTALEDLFTLQSYNHTL |
| | | | | | TQAQIEAYNVIIGGRSEESGKVKGLNEYINLYNQKNPDRKLPKFKMLYKQILSDRSSASWLPENF |
| | | | | | DKADEMLDAVNRFYADYTDHMIGVLQQALRTLPECDLERIYIRNDGAITDISNRLFGYYGVLKD |
| | | | | | ALGIDPEDKKKQEYYSIVQVQTALDAYIAESDAESNPQLFAAYSTDCIAGYFADGICRESSDICAKI |
| | | | | | EDSYQKAKDLLNTAHSGEYQLAQAEKDSLKAFLDSLMSLLHFVKPLYLKPGVELPKDDVFYGTF |
| | | | | | TPVFEQMQPLTKLYDKVRNFVTKKPYSTEKVKLNFDCSTLLNGWDVNKETQNLGVLLLKDGK |
| | | | | | YYLGIMDKANNKAFSEAESSEETDCYQKVMYKLLPGPNKMLPKVFFSRSRIDEFAPSEEVLRIRE |
| | | | | | KETFKKGPSFSLADCHTLIDFYKASIEKHPDWSQFNFHFSPTEQYEDISEFYREISEQGYKLTFKPI |
| | | | | | STSYIDSLVNDGKLYLFQIYNKDFSPYAKGKPNLHTLYWKALFDEKNLSDVVYKLNGEAEMFYR |
| | | | | | KASIAEKSKIVHQANLPIEKKNPNLKGQYSTFAYDITKDHRYTVDKFQFHVPITMNFKAGASPRI |
| | | | | | NESVCAYLKNNPD |
| MG29 | 6488 | MG29-165 effector | Protein | Unknown | MFDIFTKQYQLSKTLRFELKPIGKTKDYIEQKGLLSQDEERAEDYKRVKKIIDDYHKDFIHKSLS |
| effector | | | | | GVKLEALEEFEKLYFLPSKDDKTQKEFKKLQESLRKQIASAFRVHPTFKNLFAKELIKDELTSFV |
| | | | | | TDESDRALIEKFKNFTTYFTGFHKNRANIYTHEPKHSAVVYRIVHENLPIFLSNKKAFESVRRDYP |
| | | | | | QLLEQTQSSLLEHLEGGIVEYMFSLEYFSFTLAQKYIDLYNTMLGGKTLEDGTKIQGFNEKINLY |
| | | | | | RQANGIDKRKLPNFKALNKQILSDRESLSWLPEAFKTKEERTEAVRVFYAEHIAKFQCCDGVVD |
| | | | | | LLERIPELFTAKNMYEPSKIFVKNDLSLTAISQSLFSDYRTIREALWQKHLSQNPKAQKSKDLAG |
| | | | | | DEERFFNRKNSFFSVAEIEAALSEYASEKSMFDYFYKGAEQAASEIKAAYASWVNNPNDTKTTKK |
| | | | | | LLDAILSMQRHLKPLYVKTDTDKDIAFYAMFDTYFESLNGISKLYDKVRNFETKKPYSLEKFKLN |
| | | | | | FENSTLLDGWDVNKEPDNTAILFLKDGLYYLGIMDKKHNRVLKNIQPTTKEGGYKKVEYKLLP |
| | | | | | GANKMLPKVFFSNSRIGEFAPSAQLLENYKNDTHKKGDKFSLADCHALIDFFKASIQKHEDWKH |
| | | | | | FGFNFSPTSSYEDLSGFYREVEQQGYKIAYKNIEAEYIDTLVDEGKLYLFQIYNKDFSPYSKGTPN |
| | | | | | MHTLYWRALFDEQNLADVVYKLNGQAEIFYRKKSLDYSEEKLKVGHHPELKTFAYPIIKDRRFA |
| | | | | | FDKFQFHVPITLNFKATGGDNINALVNGFVAKNSESIKIIGIDRGERHLLYVSLIDAKGCMVEQYS |
| | | | | | LNQIINSYNGKEHIIDYHDKLTKREDERAKARVEWGNVENIKELKEGYVAKNSESIKIIGIDRGER |
| | | | | | HLLYVSLIDA |
| MG29 | 6489 | MG29-166 effector | Protein | Unknown | MQNDLVWDDLVNKYSLSKTLRFELKPVGKTLDFIKESKLIEEDKQREADFNDVKKIMDDYYKD |
| effector | | | | | FIERVLNSVIIEPSDLEYFRLVYENLKKDKYNVDLQKKYFNIQKKLREQIYDQIKTNPDFTNLFKK |
| | | | | | ELISELLPSWLKENEKLDQLENVLKFSGWTTYFTGFFENRKNVFSEKDIPTSITYRVINDNLPKYID |
| | | | | | NLSKIDKILALNDFDYSEIETKYSVELSNKKLIDFFSLNNFNCFLNQKGIDIYNLIIGGKSVENKKIQ |
| | | | | | GLNELIYLHSQKAENKEIQKSIKHLKLIPLFKQILSDRVSFSDKYEQLNNNKEVIENIGVFYNLLIK |
| | | | | | EEIFNKLKNIINSIDQSDFSQIYIKNDSNLTTISQGLFKDYSKIKLGLREYAITEKNLKTDKKIDLFL |
| | | | | | KQKYFSIAEIEQGLKLIGLNKSISEYFKSFKLDDKEIFSEITDAYDKFKSIEYDPNGDRFDIGEETEK |
| | | | | | GDLIKQFLDSIMDLLHFIKPLYVSLKNKTIDDEDEKKSEAYELDASFYSEFNEIYEKLSELIKIYNQ |
| | | | | | TRNYITKKPFVTKKFKLNFNCSSFLTGWGTGFTTNSAIIFRKSDNDKFFYYLGIVPQNLSFEDKEK |
| | | | | | LFLVSNCENVIKWVYYDFQKPDNKNIPRLFIRSKGDSFAPAVEKYNLPIEDILEIYDNGYFKTEYK |
| | | | | | KTNFVKFKNSLIKLIDYFKLGFENHESYKHFNFKWKESKEYNDISEFYLDVIGTCYNLKFIDVNFN |
| | | | | | NLIELVENKKIYLFQIYNKDYSKYSKGKKNLHTIYWEELFSKENLKNVVYKLNGEAELFYREKSL |
| | | | | | PVKITHPKNQNIDNKDPINNKKTSCFDYDLIKDKRYTEDKFLFHCPITLNFKKKDIKTINNEVNKII |
| | | | | | KSSEVNILSIDRGERNLLYYTLLDSNGNILLQNSFNSVFDDLNRKSDYKDKLDSIEGERKAARKN |
| | | | | | WHKIINIKELKEGYLSQVIYKISKIAVENNAVIVLEDLNYGFKKGRFKIEKQVYQKFEKMLIDKL |
| | | | | | NYLVFKEKDPEEIGGSLKAYQLTSKFDSFKKLGKQSGIIFYVPASYTSKICPKTGFINFIYPKFENI |
| | | | | | KQAQDLLSKFKYIKFHLNEDVFEFNFNYSDFKSKNDKTKLIKDNWSIWSNGVKLVQFRNSEKNN |
| | | | | | MWDTKEVNVTEELKKLFDNQRINYKDINNLKDQIVNVDSKEFLEKLIANLKLILQLRNSRSNSDE |
| | | | | | DYILSCVKGKDGKFFDSRSAKDNEPKDADANGAYHIGLKGLMIIDKIKEQESDKKIDLKIQNNDFI |
| | | | | | NERIKRCN |
| MG29 | 6490 | MG29-167 effector | Protein | Unknown | MEKFSIKYQLSKTLRFELIPQGKTLEHIQNKGLLTQDEQRAESYKKMKKTIDGFHKHFIELAMQ |
| effector | | | | | NVCLTKLEAFRDLYFAPTEKKKNDAFKKDLEKVQETLRKEIAKGFQTGAAKDIFSKIDKKELITE |
| | | | | | LLENWIQTQQKEDIYFDENFKNFTTYFGGFHENRKNMYSDKEQSTAIAYRLIHENLPKFLDNIRT |
| | | | | | FERIKAIPDLHEKCSVLYKEIEEYLNGITIDEVFELNYFNEVLTQKQIDVYNLVIGGRSPKEGIKKI |
| | | | | | QGLNEYINRYNQQQQDKNQRIPKLKILYKQILSDRESTSFVAEKFENSQEVLEAINGFYHHKLISF |
| | | | | | QPEGKEDTENVLTELQEILASLKEYDLSKIYIRNDKSLTDISQALFGDWSILKNALEFAYLQTIEIG |
| | | | | | KKGLSKKQETEKEKYLKQSYFSIAEIEKALFAYRNEVELLNNLKENDLPITNYFKTHFKTKKEND |
| | | | | | TDKEFDFISNITAKHSCIKGILENYPEDKALHQDKTAIDNIKLFLDTMMELLHFVKPLILPKDSTL |
| | | | | | EKDDVFYGQLNVWYEQLNELISLYNKVRNYATQKPYSTKKIKLNFENATLLDGWDVNKEEANT |
| | | | | | AVLFKKGNLFYLGIMDKNHNKIFREFPKNKSRNNYSKINYKLLPGASKMLPKVFFSEKNIAYYAP |
| | | | | | SEEILNIRNRSSHTKGGKPQEGFGKADFNILDCRKMIDFFKSSIDKHQDWKNFGFQFSDTQSYNSI |
| | | | | | DEFYREVEAQGYTISYTNISEDYINQLVDEGKLYLFQIYNKDFSPFSKGTPNMHTMYWKALFDEE |
| | | | | | NLKDVVYKLNGQAEIFFRKKSIKDDKKIIHPKQIKIAKKIFKNSDKTTERVPDETIKRLNEFYQGK |
| | | | | | IQAKDLKNEYKKYLDNFSLFNKDKNRQDIDLIKDKRFTVDKFQFHVPITLNFKATGGDFINQDVL |
| | | | | | AYLKNNPDVNIIGIDRGERHLIYFTLINQKGEILLQESLNTIKEDQHKIETPYHTLLDRKEKDRDK |
| | | | | | ARKNWGTIENIKELKEGYLSQVVHKIAKMMVEHNAIVVMEDLNFGFKRGRFKVEKQVYQKLE |
| | | | | | KMLIEKLNYLVFKDKVPNETGGLYKALQLTSKFNSFKDLGKQSGFLFYVPAWNTSKIDPTTGFV |
| | | | | | NLFNTKYENIEKARKFFDSFKSIRFNTEKNYFEFVFDYNDFTTRAEGTKTYWTVCTYGERILTFR |
| | | | | | NPKTNNQWDNEAINLTEQLEDLFGKYNIVYGKGNCIKEQIVAQTDKVFFEKLLQLFKLTLQMRN |
| | | | | | SITNSEVDYLISPIMNEKGEFYDSREEAKKGNSNLPKDADANGAYHIAKKGLWVLEQINNFKGDN |
| | | | | | WKQLKLAISNKEW |
| MG29 | 6491 | MG29-168 effector | Protein | Unknown | KLNFKTSTLADGWDLNKEKDNYAVILRKLNPITEQRDYYLGIMTSADKKIFEIKNNPSLGQNNYE |
| effector | | | | | KIIYKLLPGPSKMLPKVFFSDKNIAFFNPSEEILAIRNRGSIITKNGKTQKGFEKEEFNLEDCIIKIID |
| | | | | | FYKLSLAKHEEWSNFNFIFKQTNKYTDVSEFYAEVANQGYKISFLNFDEKYIKQLVIEGKLYLFQI |
| | | | | | WNKDFSVNKSEKNKNSRKNLHTEYWLNLFSEENLKDVVYKLNGEAELFYRKKSILKHITHLKN |
| | | | | | NAISNKNPIRDKETGELKEKSTFKYDLIKNRRYTEDKFFFHCPITLNFKAGNKSKVIHKSVNEFIR |
| | | | | | EARGQINVLGIDRGERNLAYYTLIDPERNIIEQNSFNIISDDLQRKLDYQEKLDIIEGDRDAARKN |
| | | | | | WRKINNIKEMKTGYLSQVIHKVSKLAVEHNAIIVLEDLNFGFKRGRFKIEKQIYQKFEKMLIDKL |
| | | | | | NYLVFKDRDNNQTGGVFKAYQLTNEFESFKKLGKQSGIIYYVDAYKTSKICPKTGFVDLLYPKF |
| | | | | | ENIKKSQEFIRQFEYVRYHKDENLFEFNFNYSKFASDNNKIKLARDNWSIWSNGIKLIQSKINNY |
| | | | | | WRTKEIDVTAELKELFGKNNIDYGSGNNLIEQVIKIEEKYFYESLMKALKVIIQLRNSYTDYEIKD |
| | | | | | LKKKLGDNFKGHECDYILSCVKDTQGKFFDSRNAKDVEIQDADGNGAYHIALKGLMLLDKIKK |
| | | | | | ADDAKIDLKIDRADFLNYTIERNR |
| MG29 | 6492 | MG29-169 effector | Protein | Unknown | MLNNFNHLYSLSKTLRFELKPVGETADYIEDFKSEYLKTVVAQDEQRAEDYLAVKALIDDYHRA |
| effector | | | | | YIEQSLSNVDSETGELLIAPEDFEDAYSYFQAFKAPGADRKARDEWLAELDKLRKALVRLFTGQ |
| | | | | | KELFGKGLITDELPEWLEQQGRWEENKELVGRFNKFTTYFTGFHENRKNMYTAEAIPTAIAHR |
| | | | | | LMNENLPRFFANCDNYKSLVAKYTDLELSLDEALLSKFGVSSAGDIFQPRFFINLFTQSSIEDFEEV |
| | | | | | LGGRVDEDRGKIQGLNELLNLYRQKNNLKAREAPGFTHLYKQILSDTESRSFVLEAFETDREML |
| | | | | | DAVGSYLELKSEPLSKLSSVMSALPEVDAEKVYVKGNDLTRVSQGAFGTFALIPAALTHYAETVV |
| | | | | | FPLEPDKKATKKLLENRESYGKQELYSLKELEEALSAFNESLDDQDGFSEKLKKAENHPLISYFA |
| | | | | | TATKTAEAELEEAADALKPLLSLEQLNKKRIPPEGDSEEGSEGFEQVRRIHECLDAHMSLQQVL |
| | | | | | RPLHLVLGRKPIDVASKDLGFYARFDEAIEDYNAMTIALYNKTRNHLTKKSFSTDKVKINFESPT |
| | | | | | LLAGWDLNKETANKSIILRQNGKYYLGIMHPRHPKIFSKPPEAKVGDEAYEKVNYKLLTGANK |
| | | | | | MLPKVFFSKKGLETHNPSEQILALYKNGEHKKGDTFNIESCHKLIDFFKSRIPLYKRDPSDPYGW |
| | | | | | EIFDFKFSPTKTYKDISGFYREFEEQGYKLWFTHVTKAYIDEQIEQGHLFLFEIYNKDFSPFSKGK |
| | | | | | PNLHTLYWKGLFEQNNLDDVVLKLNGEAEIFYRKHSIAANEQIVHSANKAIVNKNENNPKPESTF |
| | | | | | EYDLVKDRRYTKDKFFFHVPITLNHKAQKPVRFNDQVNRALQKADDIHVIGIDRGERHLLYYTV |
| | | | | | VNQKGEIIEQDTLNTISTDQDYVVDYHHKLDQQERTRDKARKAWTNIDNIKELKAGYLSHVVHK |
| | | | | | LAELIVKHNAVVCLEDLNFGFKRGRFKVEKQVYQKFERALIEKLNYLVFKDTTEGQPGHYLKA |
| | | | | | YQLTAPFESFKLLGKQSGILFYVGASYTSKIDPATGFINFLKPHYESLAKSKTFFESMDSICFNAKR |
| | | | | | GYFEFSFDYANFSVPQTLDDYQTAWTVCTHGETRYHNQRNDKGIWETKAVNVTEKLKVLLNEA |
| | | | | | GVSYQNGEELKDAIAAVKSSKFYRSLYFLLRLTLSLRHSVTGTEEDFILSPVADEGGNFYDSRNAS |
| | | | | | DAEPKDADANGAYHIALKGLWNLEKIDQWDGESRLNLAMKNVEWFQFASEKPFKE |
| MG29 | 6493 | MG29-170 effector | Protein | Unknown | MQTVFDQFTGLYSLSKTLRFELKPIGQTKELLEDFYKHCEGNPIVVDEQRICQYPKMKALLDDY |
| effector | | | | | YRLFIDKTLSRPIFSAEEITKAYELYCAAKQSHNSKQKESKYQKEYTAAKKSLRKKLAACFAEQK |
| | | | | | EEFGLDKYSHLFGSEQLPLNCWLQSRLQGGQITAAEYEEGLAALKAFERFTTYFTGFKENRDNL |
| | | | | | FAEVDKASAIANRVIEENMEKHFFNCRALADSTQKYPQLAEELGSFLSFFMPAYYGSCLSQQGID |
| | | | | | CYNQAIGKELNGDSTKGVNQIINEYRQKYSLKTKDLPTLITLHKQLLSKKPDCPVSETLTTDQEM |
| | | | | | LQLAKYCYTTAISRLNELQKIMSDYLNDENLQFIYLKTKDLNALSKKMFGEWDTIRNAYFYHCQ |
| | | | | | QLTDKEQKRFAANTKEVISLGLLQRLLNSYLPGSENPPPLVDPALYFKSFDLTPLRQAYEAAAPV |
| | | | | | LALTQLDQDKAPPNEGNPKGGLGYRQTILVKNLLDTILRAKDFYKPFLLEQDGKPIAVADSNELF |
| | | | | | YTQFTAAFAKLNCLYKKYNLIRSYASQKPFSTDKFKLNENNSTLLSGWDLNKEEENTNILLRKDG |
| | | | | | QYYLGILKNTKLFADCSKYLCQDSVEHYEKMVYKQVSGVNKMFPKVFFAAKNLELYKPSAQIL |
| | | | | | QIKAQKSHLKEANNPDAKNAWIDFCKDSIAKSEWPQYFKFKFKPAEQYPDVNSFYREADAQMYS |
| | | | | | LTFQNVAGAFVKQAVENGELYLFEIYNKDFSAYSKGKPNLHTLYWQMLFDEHNLCNIADNAEQ |
| | | | | | PVFKLNGEAEIFYRKASLDDKVTHPAKKPIDNKNPLNGKKTSTFAYDLKKDRRYMQNKLYFHCP |
| | | | | | ITINFRRGTLPQAVFNYQVNSFLQNNPQINLLGIDRGEKNLLYYTLINQRGQILKQGSFN |
| MG29 | 6494 | MG29-171 effector | Protein | Unknown | MGMIGDQFIGQYSLQKTLRFELRPIGETQKLLQDFKEEVQGNLLEYDAERARAYPMVKKVLDD |
| effector | | | | | YYRYFIDQVLSGFAFDSQTINEVYEMYKKAKKDAEAAKEYAVHTKKLREQLSAAFKAPITYYML |
| | | | | | DKYEHLFNRNRESRLFEWLDIRFENDHLTENEYDEIKDVLDKFDKFTTYFTGYKENRANLFVAD |
| | | | | | EKATALAYRVVNENMPRFFENCIRMENIKKRHSDLYKLLVSFEGYFVPQAYANIICQPAITDYNKI |
| | | | | | IGRPTQNPDEKGVNSIINEYRQKNQIKNRELPMMAQLYKQLLSDRITVFLDPVINNDEEMQSIVA |
| | | | | | ETIEIARGLFSEVINLTAIHALADNSENIYINSSALANLSHRVYDDWNLIYRACEAKMIKLEGKQK |
| | | | | | KGLENKLKMAIPMSELQNIIEEYIATLDEELKLSYYKIPILCNYFQNPPLDDFESATLKFEQIVKTT |
| | | | | | TPRTDLIHAIKEVLDKAMEVVRFFKPLYLFKGRSPLEVPDRNEDFYNEFERLYAELNLISKIYDRV |
| | | | | | RNYATKKQFSQDKIKLNFNNPTLLDGWDLNKEQDNLCVILIRDGNYYLALMNRDYRRLFDLKN |
| | | | | | DEVRNKALGKAGDHCYSKLEYKQVTGANKMLPKVLFAATNSDLFKPSQEILDIRKTGSHKKEA |
| | | | | | GNIEALHKWIDFCKQSIATHPEWNDHFDFKFRSTSEYSELTEFYNDFDRQAYKIKFVDIKVEYIDQ |
| | | | | | LVKEGKLYLFQIYNKDFSPYSKGRPNLHTTYWRMLFGNENLANITMDTDRPIFKLNGEAEIFFR |
| | | | | | KASLEKQITHAKGQPITNKSKKDNGKESESIFEYDLIKDKRYTEDKLFFHCPITINFRAPGTTVGS |
| | | | | | FNRKVNYFVERNPEVKIIGIDRGERHLLYYTVIDQKGNILEQGSLNQIHNSYTSAGRVVEHNINYR |
| | | | | | DLLHEKEKGREEARKNWETIENIKELKAGYLSQIVNLLSNLMIKYNAVLVLEDLNAGFKRSRIK |
| | | | | | VEKQVYQKFEKAMIDKLNYLVFKELPPGSSGHYLNGYQLTAPFTSFRDLGRQSGFLYYVYPSYT |
| | | | | | SHICPKTGFVNLLNTRYESIEKAISFFEKFNSIKYNPGSDYFEFDFDYASFGKDVARSQWCVCTAG |
| | | | | | EKRYYYANHDKTSRECNATQQIKELLDKYNIEYIRGKDLLPEIIKKNDKGFFNGLMFLLGVVLQ |
| | | | | | MRYTVSGTSNDDDFILSPVMDEQGQFFDS |
| MG29 | 6495 | MG29-172 effector | Protein | Unknown | VKDLLNTSLQPNEKLVQQKDKTLLIKNYLDSIVNLLHFIKSLKPREELSQKDEAFYGRFDELYEA |
| effector | | | | | LNVITPLYNKVRNHLTKKPYSTEKYKLNFENSTLADGWDLNKEADNTTILLRKEGNYYVAVMD |
| | | | | | KKHNKIFRDIPASAKGEAVYEKMVYKLLPGANKMLPKVFFSKSRIEEFNPSTELLENYKNETHK |
| | | | | | KGESFNIQHCYNLINFFKSSICQHEDWKHFNFNFSETGTYEDLSGFYREVEHQGYKITFVNISESYI |
| | | | | | HKLVEEGKLYLFQLYNKDFSKYSKGKPNLHTLYWKMLFDETNLKDVVYKLNGEAEVFYRKKSI |
| | | | | | EDKNKIIHKANQPISNKNPENVKKQSSFQYDITKDHRFTKDKFQFHVPITMNFKAKGILNVNNEA |
| | | | | | NKYLRNNPDTHIIGIDRGERHLLYLTLINQQGEIIKQESLNIVANEKQKTDYHKLLEAKEGKRDE |
| | | | | | ARRDWGTIENIKELKEGYLSQVVHKIAEMIVDYNAIVVMEDLNFGFKRGRQKVEKQVYQKFEK |
| | | | | | MLIDKLNYLVFKTPKPNQPGLLNALQLANKFESFKKLGKQSGFIFYVPAWNTSKMDPVTGFVDF |
| | | | | | LKPKYESVDKAIAFIKQFDAIQYNKAKQYFEFVFDYSNFTEKAEGSKTKWTVCTTNTERYVWNK |
| | | | | | TLNNGKGGQEHNNVTEKLEVLFGNAGITYGNGENIISKITEQTGAEFYKALLKLLSITVSLRHNN |
| | | | | | GEEGEKEQDYILSPVAPFFDSRKATDTLPKNADANGAYHIAKKGLWVLEQINKCEDFKKLKLAI |
| | | | | | SNKEWLEFVQKSSIKTNYPQPFA |
| MG29 | 6496 | MG29-173 effector | Protein | Unknown | MQVQKCIWDDFTNKYSLSKTLRFELRPVGKTLEYIKERGLIEVDKQREEEFNKIKKIMDEYYKE |
| effector | | | | | FIEIVLSNIEINLSDLKEYQTIYNKLIKDKSNLMLKKQYKHIQDKIRKQIYELIKKTDNYNKLFGKE |
| | | | | | LINDILPNWLGSKDRLADKELVMKFSKWATYFSGFFKNRENVFSEKPIPTSIIYRIVNDNLPKYLD |
| | | | | | NLIKFEAIKQLNNFSYSYMETELSTKLNNSSIDSFFILNNFNKHLTQKGVDLENLVIGGYSENGIKK |
| | | | | | QGLNELIYLYSQKPENKEYSKMIRKLKMMPLFKQILSDKESFSDKFDTFINNEEVIKEITDYYTKI |
| | | | | | YSQWLPELKKLITDINNFDLTQIYINSSSISSISKKLCGDWETINKGLIEYAVNKLNLKTKAKKEAF |
| | | | | | LKNKYFSLYEIDESIKLLNLENPISICNYFSKFGESKTEQADLFKTVVNNYDSFKKIVLIDKHKFSE |
| | | | | | MDIEIIKSFLDSILGVIHFIKPIYINLNIKEEDNSQDAYETDADFYVRFNEIYMNNFNRIISIYNKTRN |
| | | | | | YTTKKPYSTKKFKLNFENSQLLNGWDANKEKDYYSLLFRKDDYYYLGIMAKGHNKIFENINNN |
| | | | | | QDIDSFEKIKCKLLPDPNKMLPKVFFSNSNIDYYKPSQEIKNIINYSTHTKGGIPKDGFEKKDFNLS |
| | | | | | DCHKLIDFYKSSLYKHPEWNCFNFKFKDTNLYHDISEFYDDISEQGYKLEFSKIRENYISDLVDSG |
| | | | | | KLYLFKIYNKDFSKNKAKKNKNSNPNLHTIYWREIFSKENLDNVRYQLNGGGEVFFREKSITSNK |
| | | | | | TIHPKKTTIDAKNPIKGKFKCTFDYDLIKDKRYTEDKFLFYCPITINFKAKGSGKDIHKQINEFISN |
| | | | | | TDKPVNVIGIDRGERHLVYYTLLDPNGDIIKSGSFNLVSDDLDRKFDYQDRLDTREKERDAARKS |
| | | | | | WQSIEPIKNLKEGYLSKVVHNISKLAIENNAIIVLEDLNFGFKKGRFKIEKQIYQKFEKMLIDKLN |
| | | | | | YLVFKDIPSTKIGGSLNGYQLTNKFESFSRLRKQSGILFYVNASYTSKIDPCSGFFNMILPNYESVE |
| | | | | | KSKELLKKFEYVRYNKNEDLFEFNFNFANFNKDIKLKKDNWSIYSNGTKLVNFRNKNKNNEWN |
| | | | | | TEEVNINEQLKVLFKIYNLDYMNSKNLMNSFNRIDSKDFFEQLIGLLKLILQLRNSRINTFNDYILS |
| | | | | | CVKDKNGHFFDSRTSGNNLPKNGDENGAYNIGLKGLIILDKIKKKEELKISKEEFVNYLIDKNM |
| MG29 | 6497 | MG29-174 effector | Protein | Unknown | MLEGFSNKYPVSKTLRFELKPVGKTLEYIEKEGFIEEDESLAQSYKKMKKTIDAYHKDFISRALS |
| effector | | | | | TIALKGLSDFQDAYDQSKAFSDEATKKRLIKAQDDMRKQITSVFTNADFKDEFGNLFKKELLTTT |
| | | | | | LEKFVIEYNTNNKEKLFFVDDFKSFTGYFLGFHENRKNIYSSDAKHSTLAFRLVHENLPKFIENIN |
| | | | | | SLQAIKNNHPQLFDTLAKQVNHSLDFIFSSGFSIDDMLNIEFYDNMLSQQGIDRYNQVLGGVVLES |
| | | | | | ASKKLQGLNELINLYNQKLPKGGGVRKAPKLRPMYKQILSDRSSLSFLPEKFADDGELYDAVNE |
| | | | | | YYIGHLLAQPDNTKQATLLESLKDMVTRLNNYDLTKIYLKNDKALTHISNTLFSDYSVLPTALSY |
| | | | | | YYETQVNPSYSESLEGTKTQRKKESLIKEKDAFLKGFHSVSTIERAYREYVVSIDEDSDLRAFGIS |
| | | | | | TPVQHYFSQHFVSKPVDAEKPAVDLIQDIANKYTTIKGKLNSEVEPGTRELMQKGAEVANIKHL |
| | | | | | LDAIMSFVHFIKPLSVGGGEAGEGLDIDPSFYSEFLPLYEELGLFFPLYNKVRDYISQKPFSEEKFK |
| | | | | | LNFKNTNLLTGWDANKEKDRLSLIFRKGGHYYLGVMNKAHKKSFEDLPQATECSDDCYQKMH |
| | | | | | YKLLPGPNKMLPKVFFAASNIDYYNPSEALLENYKKGTHKKGADFNLKHCHELIDFFKQSIAKH |
| | | | | | EDWKQFGFKFSDTSSYEDLSGFYNEISNQGYKVSFIDVDSSYIDDLVESGRLYLFEIYNKDFSAKST |
| | | | | | GRPNLHTIYFKSLFSDENLADTVYKLNGDGEMFYRRSSIKPENTITHKKGERVHSKNPNTPDASR |
| | | | | | VLDYETIKDKRYTQDKFQLHLSMTMNFKASGIGPGQFNNLVKATIRDNKTKDMHVIGIDRGERH |
| | | | | | LLYLTVIDKNGKLVHQESLNNIVSGENSITPYHKLLDEREKDRGNARKNWGAVEGIKDLKKGYL |
| | | | | | SQVVHKLATLICEYNAVVALEDLNFGFKRGRFKVEKQVYQNFEKALIDKLNYLVFKDREPDDIG |
| | | | | | GARKALQLTSPFSSFKELGKQTGFLFYVPAWNTSKIDPVTGFVDLLKPKYENLDKAKDFIDRFDA |
| | | | | | IRYNKDKDYFEFVFDYDNFNNKATGTRTHWTVCSYGNKRFVYDKKANNNRGGQKTVDVNAEL |
| | | | | | KALFDGYDIDYEKLAGSDLRPFIRNKADKALYSTLMHLLKVLLAMRYSKANTSGGADEDFILSP |
| | | | | | VADENGVFFDTRDYLPANKGEEKGAYGAMPQDSDANGAYHIALKGLWVMRQIQNHEGEDFKG |
| | | | | | LDLKITNKDWLNFAQTKPY |
| MG29 | 6498 | MG29-175 effector | Protein | Unknown | MAENKAIFDGFANKYSLQKTLRFGLIPDSESKKWIEKNLVIEKDEKLAEEYKKAKKIIDKVHKAF |
| effector | | | | | IESALEKLKLNEKALTAFEKETAKTKKERDKKTIEKIQASLRNEVADSFNKDKEEFKAMFSEKMI |
| | | | | | KENAPRFCNTAEEKATMKLFDNFTTYFKEFHKNRKNIYSNEAKATSIAFRIVHENLVTFVDNLRI |
| | | | | | FAKIREGGLDLDKAEYELKSILGKEKIEKLFSIEYFNKVLSQGGIDFYNRIIGGEFEEGSRKKIRGL |
| | | | | | NELINLHNQKERGKLPRFKQLKKQILSDRKKSLDFGFLDDSELLQAIEEFYLKELSDENGKRTPE |
| | | | | | MLRALFERAEEFDIEKIHLRNDSTLRELSNKLFGDWSAIENALSEHYEKENPSKDTKKYEKDKEK |
| | | | | | WLKQDQFPIATIETALSLYEHEKVDKGKCKGLFFRQFSSFKKEEDDKENLLERLEISYNRAKPIL |
| | | | | | ENKSPGNRLASDEQAKGKLKALLDALIDILHFVKSLRLKDAAISDKDYSFYGEFDPLFERLDGIV |
| | | | | | GIYNKTRNYLTKKPYSTEKIKLNFENSVLLGGWDRNIEDTKGGVIFRKDGQFYLGIINKNNKTIL |
| | | | | | KNPPKAKDGEVAFEKMFYKLIPNPARDLHHTILSKKRSAKYKPTKTLLEKYEQKKHIKGDNFDK |
| | | | | | KFCHELIDFFKESIKKNPDWETFNFKFSETSSYEDISGFYREVGRQGYKVVFERIPASYIETLVKE |
| | | | | | GKLYLFKIWSKDFSKDSKGTPNMHTLYWRALFDEKNLKEPIYKLNGEAEMFFRKRSVEPKITHP |
| | | | | | AGKPIPNKNPDNLKKESVFKYDLIKDRRYSLDKFQFHVPITTNFGSEGQEFIDYDVRDAIKKSPVR |
| | | | | | IIGIDRGERNLLYLSMIDENGKILLQESLNKITSLYGGGKKTTDYNSLLARSEAGRDEARRDWKK |
| | | | | | IENIKEIKEGYLSQAVHKIATLMVENEAIVVMEDLNSGFKRGRTKVEKQVYQKFERMLIEKLNY |
| | | | | | LVFKKRSPEEAGGLRNALQLTSKFKSFEKLGKQSGFLFYIPAAMTSKIDPVTGFANELNPKYESV |
| | | | | | GKSKEFFSKFERISYNSKKNWFEFSFDYSNFKTKDGLEGKWVVCSTPHERFYRNGGAKGGAKG |
| | | | | | ETLPMNANEELKKLFGEFGIEYSTGDCLKKEIVSKDSKEFFKKLTRVLASILSLRQNNGKTGQDE |
| | | | | | QDYILSPVEPFFCSLDGKDGLPKDADANGAYNIARKGLLAVRQIRSAEDPKKARLAIRNKEWLEF |
| | | | | | AQAMK |
| MG29 | 6499 | MG29-176 effector | Protein | Unknown | MLNNFNHLYSLSKTLRFELKPVGETADYIEDFKSEYLKTVVAQDEQRAEDYLAVKALIDDYHRA |
| effector | | | | | YIEQSLSNVDSETGELLIAPEDFEDAYSYFQAFKAPGADRKARDEWLAELDKLRKALVRLFTGQ |
| | | | | | KELFGKGLITDELPEWLEQQGRWEENKELVGRFNKFTTYFTGFHENRKNMYTAEAIPTAIAHR |
| | | | | | LMNENLPRFFANCDNYKSLVAKYTDLELSLDEALLSKFGVSSAGDIFQPRFFINLFTQSSIEDFEEV |
| | | | | | LGGRVDEDRGKIQGLNELLNLYRQKNNLKAREAPGFTHLYKQILSDTESRSFVLEAFETDREML |
| | | | | | DAVGSYLELKSEPLSKLSSVMSALPEVDAEKVYVKGNDLTRVSQGAFGTFALIPAALTHYAETVV |
| | | | | | FPLEPDKKATKKLLENRESYGKQELYSLKELEEALSAFNESLDDQDGFSEKLKKAENHPLISYFA |
| | | | | | TATKTAEAELEEAADALKPLLVLDQLNKKRIPPEGDGEEGSEGFEQVRRIHECLDAHMSLQQVL |
| | | | | | RPLHLVLGRKPIDVASKDLGFYARFDEAIEDYNAMTIALYNKTRNHLTKKSFSTDKVKINFESPT |
| | | | | | LLAGWDLNKETANKSIILRQNGKYYLGIMHPRHPKIFSKPPEAKVGDEAYEKVNYKLLTGANK |
| | | | | | MLPKVFFSKKGLETHNPSEQILALYKNGEHKKGDTFNIESCHKLIDFFKSRIPLYKRDPSDPYGW |
| | | | | | EIFDFKFSPTKTYKDLSGFYREVEEQGYKLWFTHVTKAYIDEQIEQGHLFLFEIYNKDFSPFSKG |
| | | | | | KPNLHTLYWKGLFEQNNLDDVVLKLNGEAEIFYRKHSIAANEQIVHSANKAIVNKNENNPKPES |
| | | | | | TFEYDLVKDRRYTKDKFFFHVPITLNHKAQKPVRENDQVNRALQKADDVHVIGIDRGERHLLYY |
| | | | | | TVVNQKGEIIEQDTLNTISTDQDYVVDYHHKLDQQERTRDKARKAWTNIDNIKELKAGYLSHVV |
| | | | | | HKLAELIVKHNAVVCLEDLNFGFKRGRFKVEKQVYQKFERALIEKLNYLVFKDTTEGQPGHYL |
| | | | | | KAYQLTAPFESFKLLGKQSGILFYVGASYTSKIDPATGFINFLKPHYESLAKSKTFFESMDSICFNA |
| | | | | | KRGYFEFSFDYANFSVPQTLDDYQTAWTVCTHGETRYHNQRNDKGIWETKAVNVTEKLKVLLN |
| | | | | | EAGVSYQNGEELKDAIAAVKSSKFYRSLYFLLRLTLSLRHSVTGTEEDFILSPVADEGGNFYDSRN |
| | | | | | ASDAEPKDADANGAYHIALKGLWNLEKIDQWDGESRLNLAMKNVEWFQFASEKPFKE |
| MG29 | 6500 | MG29-177 effector | Protein | Unknown | EKTLTYQIVSEYNGLKGFLNTEHSEDYKLIQDKERVHQLKTFLDSIMNLLHFAKPLYLDKNASEE |
| effector | | | | | KNELFYTEFTPIYDELAKIVPLYNMVRNYLTKKQYSTEKFKLNFENSTLLDGWDVNKEKDNTGV |
| | | | | | ILLKDDNYYLAIMNKQNNTVFEEIPKAINPQNTFKKMNYKLLPGPNKMLPKVFFSKSRTKEFGVS |
| | | | | | EKMLENYENGTHKKGDNFNLSDCHQLIDFFKASIQKHEDWKQFDFNFSETKTYEDISGFYREVE |
| | | | | | HQGYKITFSDVSKEYINQLVDEGKLYLFKIYSKDFSEYSKGKPNIHTLYWKALFAPENLADVIYK |
| | | | | | LNGEAEIFYRKKSINIDKAVTHNAKEKLENKNPNATKKISVFDYDIIKDRRFTFNKFQFHVPVTM |
| | | | | | NFKSTGNDFINPHVNEFLKNNPDVKIIGLDRGERHLIYLTLIDQQGNIIRQESLNTIKDEKHNIETP |
| | | | | | YHLLLDNKEEERDKARKSWDTIENIKELKEGYISQVVHKIATMMIEHNAIVVMEDLNFGFKQGR |
| | | | | | FKVEKQVYQKLEKMLIDKLNYLVFKDKPANEAGGIYKALQLTNKFTSFRDMGKQTGFLFYVPA |
| | | | | | WNTSKIDPTTGFVDFLKPKYESIEQVKTFLRKFKNIYFNSTKNHFEFSFDYKDFTTKADDTQTEW |
| | | | | | TICTHGDRIEKFLNSINKWDERTVTLNDEFVTLFDKYHIDYKDSETLKEKILQQTEKAFFERLIYL |
| | | | | | LRLTLQMRNSKTGTEIDYLISPVADKNGNFYDSRKVSQNLPKDADANGAYHIALKGLWVLQQIN |
| | | | | | KTEDLKKVKLAISNKEWLQFVQK |
| MG29 | 6501 | MG29-178 effector | Protein | Unknown | MKSDFFKNFTSQYKLSKTLRFELKPIGKTLESIQSKGLLEKDEQRASSYKRVKKIIDEYHKYYIEL |
| effector | | | | | ALKNIQLSKLAEYYELFSQNKEVRNEDAFKNLKNELRKEIVKELTKGAYKEMFERLFSKELIKE |
| | | | | | DLKLWIKDRPEFQEELQFIKEFDNFTTYFTGFNENRRNMYTDQEQSTAIAYRIVHENLPKFIENIK |
| | | | | | MYESIKLKYPDLNFTPVLKDMEDLIQGKTLDEIFSLEYFNNVLSQHGIEFLNFIIGGRTREDGTKI |
| | | | | | KGLNEYINLYNQQQMEKNKRSPKFKQLYKQILSDRTSISLRFEAFENDSELLDAIEEFYQTELCE |
| | | | | | YESEGKTKNVFDEIKNLVTSIEAYDLEKIYLRNDTNLTNISQRIFGAFGVFKDAVSYYYDHVIDPQ |
| | | | | | FQTKIAKAKGEKALKKLNDLKAKWNKEYISIAVLQTALDKYMESVDDSLEIKKTYTLKVIADYF |
| | | | | | KNHFKVIDEEKKETDLVYNIKSQYLGVKGLLNIEKSENKILAQDKEKVHQLKSFLDSILELNHFV |
| | | | | | KPLNLIADALLEKDEVFYSQFAPLYEQLNKLTPLYNMVRNYLTQKPYSTDKIKLNFENSTLLGG |
| | | | | | WDVNKEIDNSSVILRKEGLYYLAIMDKDNKRIFLNVPGIENETGFYEKMNYKLLPGANKMLPKV |
| | | | | | FFAKSRIDFFNPTQEILENYKNETHKKGDTFNIDDCRALIDFFKSSLEKHEDWKQFNFKFSPTNTY |
| | | | | | QDLSGFYREVEHQGYKMSFENIAADYINKLVEEGKLYLFQIYNKDFSTFSKGKPNLHTMYWKAL |
| | | | | | FDEKNLANVVYKLNGEAEVFFRKSSIEEKNKVVHKANESIISKNPLTKGKLNTFEYDLIKDRRYT |
| | | | | | VDKFQFHVPITMNFKATGSEFINFQTNEFLKNNPEVKIIGLDRGERNLIYLTLIDQKGNITIQESLN |
| | | | | | TIKNKVRDIEINTPYQELLNRKEKERDEARKSWGTIENIKELKEGYISQVVNKIATMMVEHNAIVI |
| | | | | | MEDLNFGFKR |
| MG29 | 6502 | MG29-179 effector | Protein | Unknown | MPKKSLDQFTFQYSVNKTLRFALTDPQGDMEKFLANMREGELKRILVEDKQRAEDYKQVKKII |
| effector | | | | | DAYHREFIEEVLGQKGVLTEEDMNEYVTIYEEFKGLSRDSKNREKVIKKKRDIEKRLREAIVKKF |
| | | | | | KKNAKYKKLFNAKLITELLPQWLEERRNQKEIYDEAKYSEEKHLVEKFNRFATYFTDFHQNRA |
| | | | | | NMYVEKDQNTAIPYRIVNVNLPKFLDNYLNYEKLIQNHSGIDFSSIEKDLKGELRDLKLSEFMKP |
| | | | | | SNFLACLNQSGIDSYNTIVGGKTLEAGKKIQGINEILNQYRMKLDKSEAKKIPLMTSLYKQILSDR |
| | | | | | ESHSFLPEQFTSDQEMLKAIREFYESISETKEGEKKSLLNNIKEFLDSFPSENTDRIYIKATEITRIS |
| | | | | | HTLFAGDWALIHRALEYSKLDSQLKKEHIISIEEIETALQKYKEDIDEEDEAIKKKLGNPHPVIDF |
| | | | | | FKSAEKVEKVEETKNESNSNPTPYKKFNIFATINQHYQAAKEILKLEELHKDRLSPQKEGDKGG |
| | | | | | KGFQQVTKIKNLLDAIKDLLDLISPLYLEYKRQKIDVSDKDDRFYVELDILYDELFSIVPLYNKVR |
| | | | | | NHVTKKNRNEERFKINFDKTTLLDGWDVNKETANLGVILRKDNNYYLAIVHKKHSSVFNYVKK |
| | | | | | RGDSNNKLKIKDGLRRDIIAQNGEGCYEKMLYKLIKEPARDLPHAVFPEKKKNNFNPPSEEIQRI |
| | | | | | YNKYKKEKQFVNRAQMHQLIDFYKESIKRNIDWNGFNFEFSFTKEYNDIQEFYNEVKRQSYKID |
| | | | | | FDKIKSSYIEDKIKKGELFLFKIYNKDFSPHSKGSPNLHTSFWRLLFDEKNLKDTVAKLDGQAEIF |
| | | | | | FRPASIKKSERKIHKKDVPIENKNLNNAKKESKFKYDLIKDRRYTQDKFLFHVPITLNFSTQNKTA |
| | | | | | KQFNTEVNHFLQHNTNVNIIGIDRGERNLLYYTVIDQEGKILEQESLNIIANRIPNQNKVDSHLKC |
| | | | | | NTFY |
| MG29 | 6503 | MG29-180 effector | Protein | Unknown | MWKLGRNVRMARKKSIFDNFTNMYRLSKTLRFELKPIGKTLENIERKGLISQDEERAKSYERM |
| effector | | | | | KETIDGFHKSFIELAMSRVKLTHLSAFADLYNASPEKKKEDAFKKKFKKVQADLRTEIAKGFRT |
| | | | | | GEAKEIFSKIDKKELFTKLLEEWIKSQQEEYYFDEEFKKFTTYFTGFHTNRKNMYTEEEQSTAIA |
| | | | | | YRLIHENLPKFLDNIKTFQKIKSTPELYEKCMILFNEIAQYLDIKRIDEAFEFNYYNNVLTQKQIDA |
| | | | | | YNLILGGRTLEEGQKKIQGLNEYINLYNQKKDKKDKIPKDKIPKLKQLYKQILSDRERISFLPEVF |
| | | | | | EDGFNETASQKVLNAIEEYYKANLLAFQQDGKGETENVLEKIKSLVAQLKSYDLNKIYIRNDRGL |
| | | | | | TNISKALFDDWNMIKSALEFSILQTRKKASTKKLKKSHFSIAEIEKALFMYRNEHEALKNLKEKD |
| | | | | | HPVADYFQTYFKAAKKDGSDKEFELIANIEAKYSCIKGILNTEYPKDKRLHQDEETIDNIKAFLDS |
| | | | | | LKEFLHFVKPLSLPSDTPLEKDEMFYTHFQVYYDQLQLLIPLYNKVRNYATQKAYSTEKFKLNF |
| | | | | | QNSTLLDGWDVNKEPDNTSVLLRLNGLYYLGIMDKGNNNIFRNIPQASPGELVYQKMVYKLLP |
| | | | | | GANKMLPKVFFSKSRIHEFAPSEELQKKYQAGTHKKGEKFNLEDCHNLIDFFKESIAKHEDWKK |
| | | | | | FRFQFSDTSSYEDLSGFYREVEQQGYKITFQDVPKSYINQLVDEGKLFLFKIWNKDFSEYSKGKP |
| | | | | | NLHTMYWKALFDTENLKNVVYKLNGRAEIFYRKKSVPTDKKVVHKANEPIENKNPNNAKKQSV |
| | | | | | FEYDIVKDRRFTVDRFQFHVPITLNFKAIGNEYINSDVLEYLKNNTDVNIIGIDRGERHLIYVSLIN |
| | | | | | KKGEILEQFSLNDIITSYKNENNKDVEVKTPYRTLLDKREDERARARENWGTIETIKELKEGYIS |
| | | | | | QVIHKIVELMVKHNAIVVMEDLNSGFKRGRSKVEKQVYQKFEKKLINKLNYLVFKDKANNKSG |
| | | | | | GLYHALQLTNKFVSFQKLGKQSGFLFYVPAWNTSKIDPTTGFVNLENTRYENLEKAKWFFRNFD |
| | | | | | DIRYNTTKGYFEFEVKKYSKFNAKADGTRQAWIICTHGDRIKTFKNPEKNNQWDNKEIILSDEL |
| | | | | | KKLFDTHGIDYKNNLRDQIISQTEKKFFERLFELFKLTVQMRNSISKSTESGDDYLISPVKNKNNE |
| | | | | | FYDSRKAGDGLPVDADANGAFHIAKKGLWALQQIRSHEGEWKKLKLAISNKEWLQFVQVGAE |
| | | | | | WNRT |
| MG29 | 6504 | MG29-181 effector | Protein | Unknown | IDEYHKDFIEKALSGIRLTKLDDFYSQYILSKEQRDDNFFDKIKEELRKEIVAAFSKGELKIQFAN |
| effector | | | | | MFKKELIKEDLLNWIGDEKRNSVKEFENFTTYFTGFHENRKNMYSAEEKSTAIAYRIIHENLPKF |
| | | | | | IDNIRIYDTIKFKHKNLDFSPILNELKDIIQGKSLDEIFTLDYFNNLISQNGIDFLNSIIGGRSGKSSE |
| | | | | | KKIKGLNEYINQYNQKQNDKKDRIPKFKQLYKQILSDRSSISWMPQAFEKDTEVFDAINDFYHVE |
| | | | | | LGNAEIDGRSVNILNAVKTIVKSLSDYEELDKIYLRNDLSITTISQTIFSDYGVLGRALNHYYETFV |
| | | | | | SPQWLVDYAKAKETKRKKLEAEKEKFIKSTYISIAVLQTALAEYVKTLDDDSSIKQKYSATLIAD |
| | | | | | YFTKYFYAKDENGNEAKNTLTDQITIEYGDFKNVLDNKRSVDYKLIQDKKHVHQIKIFLDSIMNL |
| | | | | | LHFVKPLYVDKSASEEKNELFYGEFTPVYEELAKIVPLYNKTRNYLTQKPYSIEKFKLNFENSTLL |
| | | | | | DGWDANKERDNTAVILIKDDRYYLGIMDKRHNTIFEKIPETNNRNAVFKKINYKLLPGPNKMLP |
| | | | | | KVFFSEKRMPEFGVPEEIYEKYNAGTHKKGDNFNLSDCHQLIDFFKASIQKHEDWKQFDFNFSE |
| | | | | | TKMYEDISGFYREVEHQGYKITFSDVSEEYINQLVNEGKLYLFK |
| MG29 | 6505 | MG29-182 effector | Protein | Unknown | MKTLFENFTNQYSVSKTLRFELIAQGKTQEHIEKQNFLNDDEELAEKYKKAKKIIDEYHKNFISD |
| effector | | | | | KLFSFAFTPEDLRAYEHAYQAMKKDKKNIKLMNDFASRQDDLRKRIAKELQEDNYLYAKEGEK |
| | | | | | KKFIKQNLIKWLENNSISISGIPDPKTIINDFENWTNYFSGENDNRKNIYSEKRIGTSIGYRLVHDN |
| | | | | | LPKFLENIKRYNKAKTAGVNFSNVEKTYGVKLDEVFSLEHFNKCLTQKGIDEYNLIRGGQSKKG |
| | | | | | NNKEQGINETINLHVQQLHRRLERATDDDKKELADKIKYIKSCLLEELYKQILSDRSQLSFRLDSI |
| | | | | | KNDGDLCRQIEHIFHMDNAGNLWGKKENVDRDTGEKTESDFNISEAIKKVLQAFKEADPERLYI |
| | | | | | RNKSSNAITEISQYLFGNWSLITRSLDYYVKKIIFPNPEKKKETAKQKVDREKKCEGWLKDTTYF |
| | | | | | SFAEIHTALELFFQQYSDEELLDEKKENNDEEQGITKAMKEIALSKPLFSYFKELKISKKNEETNK |
| | | | | | FEKKELLKQIKESYPATQKVFEQYRDVEAELLKNKKDGEVQVVKNYLNALMDLHHYLKPLYV |
| | | | | | MLSPKDEKNQEEIFEKDNGFYDDFDPLFNVLKLIVPLYNQARNYLTKKPFSVEKYKLNFGHHNL |
| | | | | | LGGFVDSYTNSDNATQYGAYLFRKKHQRYDAYEYFLGISTDAKLFRCHLQNDISENDKSKYERL |
| | | | | | EYYQPKSTTFFSDEYSSKNKKKIMDCLLKKVEAKIEKTLKGNSDFDEFLEKLREADTPGKQIEIIK |
| | | | | | KQKEFINILDEKEVEEIFNEAIAEMKNFIIKYVERNPQLIKLQETRYSGHEGFTAIIKDLQKIAKEN |
| | | | | | KVFNYFHVSENEFNEACNRETKPLLFFKITNKDLNYYEKAAQGLRKQEIGTKNLHTLYFEQLLS |
| | | | | | GKQQVIDIGKGEVFYRRKTPFYKPIIHEENKSILCRTYKEDGKLKTLTDDIYKELSLFYKGNIPEE |
| | | | | | KLSEASRKLKDLVKVNKFKYKIMKDKRYTEDKYLFHLSINLNFDQPPKLEKFNDSVNDSLKRND |
| | | | | | NVKILGIDRGERHLVYYTLIDDKGNIVKDDNGNYLQGSLNNPTGKKDYHDLLDKREDERDKAR |
| | | | | | KAWNTIDRIKDIKEGYLSQIVHNIASLMIKYNAIIALEDLNFGFKKGRMKVEKQVYQKLEEKLID |
| | | | | | KLNYLVFKNECPEKPGGLLNAFQLTAPFKSFKAMGKQTGFIFYVPAYHTSKICPATGFADLLYP |
| | | | | | RYKTVDKSKKYFAKFEAIRFNRQENYFEFEIKNYNAFNPKAEDTKQDWTICTYGLRLDTYRNPK |
| | | | | | KNNNWDTQEVDLTKELQNLMQSQGIQFIDGKCIKEEICRQNDSQFFKRLMKLLRLTLQIRNSRV |
| | | | | | NSDEDWIISPVRVQGEIFDSREADESMSQNADANGAYHIALKGLWCLRQIREAKDLKKVNLAISN |
| | | | | | KEWLQFVQDRPFAK |
| MG29 | 6506 | MG29-183 effector | Protein | Unknown | MPEVFQTDKEVLEAINNFYCVGLENFEIDGKSINLLDTVETMIKSLSSYKDLAKIYLRNDTAITSIS |
| effector | | | | | QTLFADYSVLGQALNYYHETFINPKYKTDYAKASETKRKKLDKEKDNFTKSAYLSIVTLQTALA |
| | | | | | EYIKTLDDDSDIKQKYSSTLIADYFINHFYAKDENDNKTKKTLTRQIANEYSILKDFLNTEQSEDY |
| | | | | | KLIQDKEHIHQLKTFLDSIMNLLHFIKPLYVDKNASEEKNELFYAEFTPVYEELAKIVPLYNMVR |
| | | | | | NYLTKKPYSTEKFKLNFENSTLLNGWDVNKEKDNTAVILLKNNSYYLAVMNKQYNTVFEKIPKI |
| | | | | | ENTQTIFRKMNYKLLPGANKMLPKVFFSKSRMEEFGVSEKMLKNYKAETHTKGNNENLSDCHQ |
| | | | | | LIDFFKASIQKHDDWKQFDFNFSDTKTYEDISGFYREIEQQGYKITFSDVSEAYINQLVDDGKLYL |
| | | | | | FQIYNKDFSEYSKGKASLHTLYWKALFAPENLADVVYKLNGQAEIFFRKKSINAEKTIVHKAKEE |
| | | | | | LENKNPLASKKNSTFDYNITKNRRYTVDKFQFHVPITMNFKATGNDYINPQVNEFLRNNPNVKII |
| | | | | | GLDRGERHLIYLTLIDQQGNIIQQESLNTIKDEQHKIETPYHKLLEKKEDERDKARKSWNIIENIK |
| | | | | | ELKDGYISQVVHKIAKMMIEQNAIVVLEDLNFGFKQGRFKVEKQVYQKLEKMLIDKLNYLVIKD |
| | | | | | NKVNEAGGIYNALQLTSKFNSFKDMGKQTGFLFYVPAWNTSKIDPTTGFVDFLKPKYESIEQAK |
| | | | | | TFFDKFKNICFNSTKDYFEFSFDYKDFTTKADGTRTEWTVCTHDERIEIFRNPNKLNQWDTRNIV |
| | | | | | LSKEFTEFFDKYHINYRKGENLKEQILKQNEKVFFEKLIHLLRLTLQMRNSKIKSEVDYLISPVVD |
| | | | | | KSGNFYDSRKVAKNLPQDADANGAYHIALKGLWILQKGL |
| MG29 | 6507 | MG29-184 effector | Protein | Unknown | MSAQSALSTLINKYSLSKTLRFELIPIGKTKESIDRKGLLSQDVKRAQSYKEVKKIIDEYHKEFIEK |
| effector | | | | | SLINAKLKGLEEFSKLYYKLQKEDKDKKNIKKMQDNLREQISDLFKNNKKDKWNILFKEDLIKK |
| | | | | | ELPLFAKDDKQKNLINEFNKFTTYFTGFHKNRKNMYAEEEKSTSIPYRIIHQNLPKFLDNIRIFEKI |
| | | | | | KKNKINTDVIEKELSLFLNGIKINDIFSINFFNDVLNQKGITFYNTILGGVSEKDRTKIKGINEYVNT |
| | | | | | EYNQKQLDKRSKIPKLKQLYKQILSDTETASFVLEQFENDNQLLEKIEQFYNTELINYETEGKTQ |
| | | | | | SVFLQFEQLFKNMQNYDASKIYISNLSIANISKIIFGDWSIICNALAEWYDKHNTKGKKINEYKKE |
| | | | | | NFLKQDFSIQQIEDAVLEYKNDTLNKEINFLLNYFASFLNEKSKKNIIQRIETEYSKVKDLLNTDYP |
| | | | | | EKKKLASDKDNVSKIKAFLDSLMDFLHFVKPFNIKKDTGLEKEENFYSIYVPLFEQIDKIIPLYNK |
| | | | | | VRNYLTKKPYSTEKIKLNFENSTLLDGWDLNKESDNTSVVLRKDDLYYLGIMDKKHNRIFKELP |
| | | | | | SQNGNESSYEKMIYKLLPGPNKMLPKVFFSKKGKKQFKPSKKLLKKYEDGTHLKGDNFNINDC |
| | | | | | HNLIDFFKESIAEHEDWKQFDFKFSSTSSYKDLSNFYKEVEKQGYKITFQNISENYINQLIDEGKL |
| | | | | | YLFQIYNKDFSKYNKGTPNLHTLYWKMLFDNDNLKNIVYKLNGKAEVFYRKSSLILGDNIVHK |
| MG29 | 6508 | MG29-185 effector | Protein | Unknown | MEALHMNNFYSEFVNKYSLSKTLRFELIPQGKTLENIKTKGFLEEDEERAKRYEKVKKIIDEYHK |
| effector | | | | | DFIEEVLSAVNLSQEELKELAKAYEGAKNKDENSKKALVDLQNKARKKIAEFFTKQEKFKTLFA |
| | | | | | KELIKNDLDSWLDEKYAEKEASEKKEVLKDFKTFTTYFTGFHENRKNIYKDKEQSTSIAYRIIHD |
| | | | | | NFPKYLDNAKKIEILLKEFPDINLKQIERELNDILQDKNLKDFFALENFSAFLSQSGINKYNSIIGG |
| | | | | | KTLEDGTKLQGINEAVNLYRQSKDHLLKTVAETEAQNLKSEIKKLRSLKLVSLYKQILSDQDSLS |
| | | | | | FLPEKFEDDKTIIDAINNFYEENIINIKIEESKEPQNLFNELQIILDHLKDADLDKIYIKNDKAITDIS |
| | | | | | QYLFADFDLIKKALEYYVKNELIKAKKQDLSTKKEEESWDKWVNKTSYFSISTLNKALIYYKEN |
| | | | | | LEDGNEDKKKFSDFKTISQYFLNTIKEKINACIENHKKTVSVLNAEYGSNSHDLITDKKIGGRVEF |
| | | | | | IKNFLDSLQTLLHFLKPLYIAPIKNEDKKAADILEKDSSFYTDFDIYYEKLNDLVPLYNKVRNYLT |
| | | | | | RKPYSLEKYKLNFKNSLLADGWDQNMESAKTSILFIKNGIYYLGIMDKNHNKSFQNIPSCSDINK |
| | | | | | PYQKIVYKLLPGANKMLPKVFFSDKNIKDYQPSEELLNNYNNGTIIKKGEYFNKAIICIIALINFFK |
| | | | | | ESIKNHPDWKHFDFKFSDTDTYSDISQFYREVESQGYKISFENVAETYINTLVDEGKLYLFQIYNK |
| | | | | | DFSKYSKGKPNLHTLYWKALFDEHNLEDVVFQLNGGAELFYRKLSIKTENRTIHKANKILENKN |
| | | | | | NLNNKKTSSFPYPLIKDKRFTEDKFSFHVPIKINFKSVNTGRENDQINKYLKSNPNIRILGIDRGER |
| | | | | | HLAYYTLIDQKGNIKKQASFNIITDYYQSKEYKKDYQVKLTEAEIARDKARKSWGSIENIKNLKE |
| | | | | | GYLSQVVHQICTIVIENNAIVI |
| MG29 | 6509 | MG29-186 effector | Protein | Unknown | MAENSIWAEMTNQYSLSKTLRFELKPSDKTEEFIEKNGLINEDEQRNKDFLYSKELLNEYYSHFI |
| effector | | | | | EKRLGEIQIDVGVLKDYFETYKEFNKLKQKGKEVDSKELKKAEARLLASQNHLRKALHRVFFM |
| | | | | | NKNISDKRDEKDIIAAVKYHADNGVVNEKVDRLNRFLIDKCTTYFTGFFNNRDNVFTSEEIPTSVF |
| | | | | | YRTINENLPFFVKNIQKYEKLKDIIPEKELITRENDLKKELGEFSVKEVFSIKYFNSCLNQKGIDKY |
| | | | | | NQILGGVKKENIQIKGLNELINELSQNKDKSIRKLKMTPLYKQILSKSESKSFKIDTIKNGKELNY |
| | | | | | KINEFYELVSTSNKDQKEALISLLEKLFKNERELDFDGIYVNSKGLRELSNKVFGDWYVIESALK |
| | | | | | QQYKSKILSTGKKEKDDSKKDKETSEWFKKTKQFSINDINSSLEAAQNDLITNKNKNIWAYFKRL |
| | | | | | ENQKELNLITEINESFKDLESVQFGEDKELLNDSNEENVKKIKKALDSVQELLWFISPLMYDKPK |
| | | | | | EEVFDLNLDPEFYGQFNIIYEGVRQIVPLYNKTRNFIAQKPFNESKFKLNFENSTLLDGWDKNKE |
| | | | | | PDNWSILFRKDNNYYLGIIASGKGNNRIFEKIPEYKQGDCYEKMNYKLLPDPSKMLPKVFFADK |
| | | | | | NIEYYSPPKEILSIRNHSSFTKGGTPQEGFEKKEFNLKDCHKMIEFYKECISKHPEWNEFGFKFKK |
| | | | | | TSEYSDTSEFYKEVSDQGYKLTFQKVSTNYLDEMVNTGKLYLFNIWNKDFSKFSKGKPNLHTLY |
| | | | | | WKEIFSENNLKDVVYKLNGQAEIFYRKKSLSGKVTHLKNIPIKNKDSINGKETSVFKYDLIKDKR |
| | | | | | YYQNKYLFHCPITLNFKAKGSSFINNKINEFIIKNCNKINILGIDRGERNLLYCSLLNSNGKIILQKS |
| | | | | | FNVMQDKFGRNVDYYNKLDAKEKERDKSRKEWKNIENIKELKEGYLSQIIHELAKIIIEHNAIVV |
| | | | | | LEDLNFGFKRGRFKFEKQVYQKFELKLIEKLNYLVFKDKNSTLAGGLLHAYQLTNEFDSFKKLG |
| | | | | | KQSGVLYYVGANYTSKIDPKTGFANLLHPSYENVEQSKEFFGKFDSIKFNKTEGLFEFDFDYSNF |
| | | | | | NKDSKLKKNQWKVFTNGERIINERTKNNTYESKKINLTDELKKLFEEEKIPFTETNNLKGHIISSN |
| | | | | | SKNLHKGLTNLLKYTLQLRNSNNKTNEDYILSCVKYDEKNFFDSRNAKESEPKDADANGAYNIG |
| | | | | | LKGLMLIEKMKEQSKKQRGEKQKYDLKISNEDYFNWVTNRNQS |
| MG29 | 6510 | MG29-187 effector | Protein | Unknown | TKDLLDALLDLHRLFKGLKIDDAEKDLVFYSYFDTYVDALSAIQPLYNKVRNFKTKKPYSQEKF |
| effector | | | | | KFTIGKSNLLNGFVESKTDKSYNGTQYGGYLFRKKRNDGRYNYYLGISSKANLFSFYGEELSPED |
| | | | | | VCEFERLHYYQVKTNSIYGSSYDGDFSQDKETLPEYEVIEKLQNVLKIKYQHVKSLKEIANRSYPS |
| | | | | | LKSLQDDIEIAVEEKSFIYKPVSQSEMEQVLSEGSMLFFWIYNKDFSEKKKNKNSRDNLHTMYFK |
| | | | | | ALMRENSNNDVFDLGKGEIFFRRKSINYDDYIWENGHHHKDLKDNFEYPLIKDRRYAVDKFLLH |
| | | | | | LSIFINYKKPNNMNINSKVNDFISKQSDKIKIIGIDRGERHLLYLSLIDAKGKIIEQYSLNQIVNSYN |
| | | | | | GKEFHLDYHGKLTEKEKKRDEERKSWKSIENIKELKEGYLSQVIHKIATLILEHQAIVVLEDLNF |
| | | | | | GFKRGRFKVEKQVYQKFEKMLIDKLNCLVDKKREWSEPAGVLNALQLTNKFTSFEKLGKQSGI |
| | | | | | LFYVPAWNTSKMDPVTGFVNLFNTKYQSVEKSREFFSKFRSIRYNEEKGYYEFEFDYNDFHDRA |
| | | | | | QGTKSHWTVCSYGKRIVNFRNKEKNNQWDWKEVDPTQELKELFGSAKGDLKEFIESHDKKDFF |
| | | | | | EKFLKIFSRILQMRNSATGTDIDYMLSPVANEKGNFFDSRKSDGSLPLDADANGAYNIARKGLML |
| | | | | | VEKIKNADDKSKVKLTVTN |
| MG29 | 6511 | MG29-188 effector | Protein | Unknown | MNQLLKRFTNQYQLSKTLRFELIPQGKTLDFIKEKGLLSQDEQRAQSYKEMKGTIDEFHKYFID |
| effector | | | | | LALSKVKLSFLSDYDCLYNQSVESKKDPKFKDELKKVQDALRKEIVKSFSEGDAKGIFAILDKKE |
| | | | | | LITVELEQWFANQDKKEIYFDDKFKTFTTYFTGFHQNRKNMYSTEANSTAIAYRLIHENLPKFLE |
| | | | | | NAKSFAKINQVLELHPQISNVYKEFEKYLNVKSIDEMFELDYFNEVLSQKGITVYNNIIGGRTAEE |
| | | | | | GKSKVQGLNEIVNLYNQNKDKKDRLPKLKQLYKQILSDRISLSFLPDAFKDGKQVLQAVFDFYK |
| | | | | | VNLLSYTIEGQDTSQNLLQLIKQLLQNLSSFDIDKIYLKNDNNLTAISQQLFGDFSVFTSALNYRYE |
| | | | | | TLINPKFELEYLKANEKKRESLDKAKAIYTKQDYFSIAFLQEVVSDYMNSLDDNSDLSKKFTPNCI |
| | | | | | ADYFTNYFIAKNEEDSDKTFDLLANISAKYQCIQGILENASEYEDELKQDQILIDNLKFFLDSIMEL |
| | | | | | VHFIKPLQLKSESITEKDTAFYDVFDNYYETLNLLIPLYNMVRNYVTQKPYSTEKIKLNFENKNNF |
| | | | | | LGGWVDSKTENSDNATQGGGYLFRKKNKIGEYDFFLGISNDVKLFRSHLKSEITENDLSEFERM |
| | | | | | DYYQLKSASVFGNSYVGNYEDDKNNLFEVIYNFAKEKNKNILNEFDIYINKQNGDIKPTPSGLFKI |
| | | | | | VNEKFSFFVNELLNFEDFNNANNLIINNLKQTILSLHRVPKSQTYKDINFKIFTEPIKVIEDLTNEKS |
| | | | | | FSYFPISKIEFEDVTNRSNNPLFLFKISNKDLSFSDSLDNGLRKLQKRGKDNLHTLYFKALMQGNQ |
| | | | | | NIYDIGTAEIFYRKASISPKNIITHKVNRPINAKNPLTPEAKNTYEYDLIKDKRFTEDKFQFHLSIIQ |
| | | | | | NYKEPKKPNKDINQTVLEYLKNNPEVKIIGLDRGERHLVYLTLIDQKGNILKQESLNVIKVDKHE |
| | | | | | THYHTLLHNKELERDKARKNWGTIENIKDLKEGYISQVVHKIATMMVVENAIVVMEDLNFGFK |
| | | | | | RGRFKVEKQIYQKLEKMLIDKLNYLVLKNKQAQELGGLYSALQLTNKFESFQKMGKQSGFIFY |
| | | | | | VPAWNTSKIDPTTGFVNYFFTKYENVEKAKSFFEKFDSIRFNGTYFEFEVKNYSNFNPKAEGTQQ |
| | | | | | NWTICTHGERIITFRNTEKNSNWDSKTVLLTELFEDFMGKHQIIYGNGECIKTQLDSKNDKLFFE |
| | | | | | ELLNLFKLTLQMRNSVTGTDEDYLISPVKNAQGIFYDSRKADDNLPKDADANGAYHIAKKGLWI |
| | | | | | LEQVNKTQSAEELKKLNLAISNKEWLQYTQQ |
| MG29 | 6512 | MG29-189 effector | Protein | Unknown | VKNASLSDISSRVFKSHGLIKAALTHHAESVYVPTAPSAKVTSKLQKQRDDYLAQDVYSLAELDQ |
| effector | | | | | VVSSYLSGLEEEASWDGVDKQKPISMYFVSAIKKAIEEKDESGTTFNMHLEAAKPLFALDALSKQ |
| | | | | | RNMPKNESDFGGEGHQQIQKIHALLDAMMVILHVAKPLHLVNNKKPIEIFDIDSEFYGEFIEVYE |
| | | | | | KYSYELVDLYNQTRNYLTKKSFSTDKIKINFDAPTFMDGWDLNKEIDNKAFILKDGGNYYLAVV |
| | | | | | CKDGRDISFDFDEKFDGDFFEKLNYKLLSGANKMLPKLFFSKKGVDKFKPSTEIIEIYKSGSFKKS |
| | | | | | EASFDIRKCHKLIDFFKENLPSYKVNDSDKYGYEVFDFKFSKTSDYKDISEFYKEVERFGYKTWF |
| | | | | | SKVPKSYLDDCVRTGKIAMFQIYNKDFSEHSSGSPNLHTMYWRGIFCQENMDNPTIKLNGEAEM |
| | | | | | FYRKHSIQKNERTIHRSGENIANKNSNNHKKESLFEYDLIKDRRYTKDKFFFHVPVTLNFGASSPG |
| | | | | | WFNDKINKAIKPEKDIHIIGIDRGERHLLYYTVINQKGEIIKQGSLNEIETDQGYSVDYQLKLKAK |
| | | | | | EDQRDAARKSWSTVENIKELKEGYLSQVVHKLSKLIVEHNAIVCLEDLNFGFKRGRFAVERQVY |
| | | | | | QKFERALIQKLNYLVLKDRKHGEPGHYANAYQLTAPFESFEKLGKQTGILFYVPAAYTSKIDPM |
| | | | | | TGFVNLLDTRYESQEKSKDFFGRENSILYNKAKDYFEFCFDYKKFPARVDFTGCRTAWTACTYG |
| | | | | | DVRYANQKNNHGKWETKSVNVTEELKKLFDSASIKWDVGCNIKDDICKADDKNFYSTLSWLLK |
| | | | | | LTLSLRYSKTGTDDDFVLSPIAGADGNFF |
| MG29 | 6513 | MG29-190 effector | Protein | Unknown | IENNLQCSFEKIMTINSFVDFMSQEGIDKYNTFIGGSPALDGGEKVQGINEFINLTRQQSGAKRAQ |
| effector | | | | | FPPLQEFYKQILSKSDISFISDFTNDKDMFEAIKIFIIELYFNEKDESGISFFNRVIKDSRELEKELSF |
| | | | | | EKENLYLPKDKVTFVSKKITGHWNNINNELLEQLGEKEFNSRKYFSFSEIEKAVNDGGQAERFV |
| | | | | | VASNSSSLITLFNEELVKNLRDSNQAWVELIETGVLDCGKLDSNRSRESDKGFIQIAAIKRFLDSVL |
| | | | | | GLSGFVRDWQASKDILKIETRNKHWYDHINDFISQFQVIDLYNMVRNHATKKPASKEKLKINFE |
| | | | | | NSTLLAGWDRNKESDNYCALFQKKGLYYLGVMTPTSNKIFDYDKKEDLRNAVLAKKGEECYR |
| | | | | | KINYKLLPGPNKMLPKVFFAKSNKELFNPSDEIIKIKENKLYSKSATAVNGLDDLYKYIDFCKKSL |
| | | | | | VKHPDWSKSFGFKENSFTETYEYVSVDQFYNEVETKGYSITFDNIKEDYINKRVESGELYLFQIYS |
| | | | | | KDFSQAKKKKGTDNIHTIYWKGLFATENLEKPILKLNGQAEIFFRKASIKYDPDVMQKGHHHEK |
| | | | | | LRGRFDYPIIKNKRFTMNKFFFHCPITLNFGSPSSPYRFNDMIRSFVRNNSDVNIIGIDRGEKHLLY |
| | | | | | YSVVNQKGKIVEQGSLNKISNGFKPKGEDKERLIDYHSKLDKMESDRDKARKSWSTIENIKELK |
| | | | | | AGYLSQVVHKLSELIVKHNAIVVL |
| MG29 | 6514 | MG29-191 effector | Protein | Unknown | TSEYNGLKGFLNTEQTEDYKLIQDKERVHQLKTFLDSIMNLLHFVKPLYVDKNANEEKNELFYA |
| effector | | | | | EFTPVYEELAKIIPLYNKVRNYLTQKPYKIEKFKLNFENSTLLAGWDVNKERDNTAVILIKDNYY |
| | | | | | YLGIMDKQNNTVFEKIPAIKNTNTVFKKVNYKLLPGANKMLPKVFFSESRIAEFGVSEKMYENY |
| | | | | | KIGTHKKGDNFNLSECHQLIDFFKSSIQKHEDWKHFDFKFSPTKNYEDISGFYREVEQQGYKITF |
| | | | | | SDISEEYVNQLVDEGKLYLFQIYNKDFSPYSKGKPNLHTLYWKALFDPKNLADVIYKLNGEAEM |
| | | | | | FFRKKSINPQKSVVHKAKKNLQNKNPLATKKTSIFDYDIVKDRRYTVDKFQFHVPITMNFKATG |
| | | | | | NDYINPKVNAFLRNNPDVNIIGLDRGERHLIYLTLINQQGHILQQESLNTIKDEQHHIETPYHKLL |
| | | | | | DGKEIERDKARKSWDTIEPIKELKEGYISQVVHKIAAMMIKHNAIVVLEDLNFGFKRGRFKVEK |
| | | | | | QVYQKLEKMLIDKLNYLVIKDNKATEAGGLYKAFQLTNKFSSFKNIGKQTGFLFYVPAWNTSKI |
| | | | | | DPTTGFVDFLRPKYESIEQAKTFLSKFKNICFNSTENYFEFSFDYKDFTSKADDTQTEWTICTHGN |
| | | | | | RIEKFRNPINKWDAKTITLNDKFEELFNNYHIDYKKEGSFKETILKQTEKTFFKQLIDLLRLTLQ |
| | | | | | MRNSKTEKDETGKDIDYLISPVTNKSGNFYDSSKASKNLPQDADANGAYNIARKGLLILQQIKET |
| | | | | | KTDGDLKKLKLAISNKEWLQFAQNQILQIDS |
| MG29 | 6515 | MG29-192 effector | Protein | Unknown | MSIFKDFTKQFSLSKTLRFELKPVGKTEEWIKKHGVISVEEDKLEGKDAKRAEHYKYVKILLDN |
| effector | | | | | MHRLFIEEAFERVENDFLIQHLKSISQENDYSFTDDRTLKKIFQEILDKTAADWMKEYSIEMPEI |
| | | | | | WQKELAGLNEKINTESDKKRKKGFESAAKALKNKIENPLKDFKKTDSSILYSNVESIQLLEWKII |
| | | | | | KGEVTATKNEFNPENNDNSQIPADKLCEYTRSFRQFYTYFTGFNENRQNVYDISGKKSTSILNRIF |
| | | | | | NDNFFFHLDNITKWEKVKENLKNENISLVLKEMSYDWKKELSEIEKNLNCSFDEALSVESFVKF |
| | | | | | MNQSGIERYNILIGGNPALDGSRKIQGLNEFINITRQKAGAKRAQFPPMQELYKQILSKSDKTFIP |
| | | | | | EFSDDKDMFVSIKSFNENSFSGSSFIDEFIKINSELQNELQNERSNLYLSKEKITSISNKITGNWNNL |
| | | | | | NYELLEQLGENIFNSRKWFSFEEIEKAVTDGKIGERFILSNTYSNSLLSFFDNEFKTLAAKAQTFW |
| | | | | | TELEKTGLLNEEKIDSNRSNEGDKGFEQIATIKKFLDSLIELSGFVRDWQTSKEILNIETRNKDWY |
| | | | | | EHLDNFVNQFRIIPLYNMVRNHLTKKPVSKEKMKINFENSTLLAGWDKNKESDNYCILLEKDGL |
| | | | | | YFLGVMTPESNRIFDYEKNERTRKIVLAENSERCYRKINYKQIASVSKDIFTLCWDENSQSAIRKT |
| | | | | | KGREEVWGERITKIKDEKLYNESLEDKEYYFSYLIKCAKSYWKYFNLSLKKPSEYKNLSELFAEI |
| | | | | | ERQGYSITFDNIKEKYIHEKIENGELYFFQIYSKDFSQNKKQKGTDNIHTVYWKGLFSPENLKNP |
| | | | | | VLKLNGQAEIFFRKASLKYDPEKMEKGHHHEKLKGKFSYPVIKDKRFTQNKFFFHCPVTLNFGA |
| | | | | | PSSPYKFNDSVRSFLKNNPDINIIGIDRGEKHLLYYSVVNQRGKIIEQGSINSIDNGFVPKGEAEVR |
| | | | | | KIDYHSKLDKIESNRDKARKSWSTIENIKELKAGYLSQVVHKLSELIIEHNAVVVLEDLNYGFKR |
| | | | | | GRFGVEKQIYQKFEKALIDKLNHLVFKKESSEKPGGYLNAYQLTNKFESFQKMGKQSGILFYTT |
| | | | | | ASYTSTTDPVTGFLKNLYPSYQNVEKSIEFWKSFDSVKYNSAKDRFEFTYTIGKVPTKNMYKEKD |
| | | | | | EDKVDKKQWTVCSCVTRSRYVKALENQTDEQKQNTSSEQIGNKGRHETFFVTDEIKKVLDGSGI |
| | | | | | DYKSSSDLKAKFLEQDTKKFHSSMIYLFNAIMTMRVTDSGKEKGTKENDFIQSPVEPFFDSRYAK |
| | | | | | EDQPENGDANGAYNIARKGLCILQNINKADDVSKADIAISKQQWQNFVQKH |
| MG29 | 6516 | MG29-193 effector | Protein | Unknown | MESLTNLYPVSKTLRFSLIPQNETLHNIEKAGILTEDEKLAEDFKKVKKIADDWLKNFINESLTGA |
| effector | | | | | SLNLEDLLIFEEKYNLFPRNEKDEEEFINIKTKLRKEIVSYLTKNPKFKLLGTADLIRKELPEFVKT |
| | | | | | EEEKDIINKFKNFTTYFTNYNQTRKNIYSDEEKHASYAYRIIHENLPLFIINKKNFNTIKTSYPELIN |
| | | | | | DIKKTTEPLLDNTKIEDMFSIEWFSNTLTQPGIDLYNKMIGGESLENGKKIQGFNEKVNLFRQAN |
| | | | | | KLDGKSVPMIKQLRKQILGDKNTPEWITEGFKDKNSMNEAIVKFIKNTEHAKNNLINKLFIKNKP |
| | | | | | YDYNKIFIKNRFIANISHELFKDWNLLKNNMLEQYKSKNPKSKNPEKEFAKIPYFSIAEIQKSIPNQ |
| | | | | | NKHFPDFIIDHFHDKIMTLIPTEKKIHELWKDNKDSIPALKNLMDYYLELYRISKPFDVDCADKD |
| | | | | | PVFYELFDEIFNDFSKVVKLYNEVRNFITKKPYSLEKIKLNFGNSTLLSGWDVNKETDNTAVLLQ |
| | | | | | KNGNYYLAIMNKDHNKIFKNTPVTKNEKNSYKKMEYKLLPKSYMMLPKVFFTKGNKDKYEPSE |
| | | | | | EIIRIYEEKTFKTGNNFNINDLHKLIDFYKESIKKNPEWSCYNFNFKPTKEYQKINEFYEETDTQG |
| | | | | | YNITFKNIPAEYIDNLVKKGQLYLFKIYSKDFSSYSKGTPNLHTLYFKMLFDDRNLKNTVYKLNG |
| | | | | | GAEIFYRKKSVTYPKNIMEKGHHAEDLKGKFNYPIIKDRRFTFDKFQFNISITLNPNAQGHGNIND |
| | | | | | ICRNLIKSNNTNIIGIHRAENHLIYITVLNSSGDIIEQRSLNEIKSYGDKTVNYMEKLTERGKERND |
| | | | | | ARINWETIGNIKELKEGYLSNVISEIANMMIKYDAICVMEDLNYDFIRERSAIEKQIYQKFEKMLI |
| | | | | | DKLNFYVDKKKSPEETSGILKPLQLANKFTTFEKIGKESGMIFYVSPYKITDIDPVTGFVNLFDTR |
| | | | | | YFNIEKAIQFFDKFDDIYYNAKTDLFGFNFDYDNFINKEKIQNTKTKWTVYSYGERIERFNTNNIP |
| | | | | | KFKKIDLTKEFKNLFSEYSINYNSNLKKSIISLKDKNFFMKLLSLFRLTVQMRNGDFIISPVNDNLG |
| | | | | | NFFDSRNSDNKTLPENTAANGAYNIARKGLILLNRIKQTSDIRKTNLKISGDEWLQYTQNKNSYI |
| MG29 | 6517 | MG29-194 effector | Protein | Unknown | MKNFTNIYPQSKTLKFELRPYGATLDNIHKSGLIDQDETLKADYHAVKKMIDEYHKVVIDESLTN |
| effector | | | | | FKLTDLPAYEELYYKSRTEVEDKEFEIIQSNLRKQIHKAFSENKRFKSIFKKELIQKDLPAFVKKE |
| | | | | | EEREQISRFYHFTTYFTGFHENRKNIYTAEAKATSVCNRLIHENLPKFLDNRKTYLNYISNFIDLD |
| | | | | | LSQVEEDLQEVLGDITVDDLESLDSFNHTLTQRDIDIYNLALCGRSIEGEKKIQGINECINIYRQKN |
| | | | | | RLKARQLPNIKPLYKQILSESKSGSFLLDKFEKDEDLFDSLRNFYHCLNSFNYKGEQDKSTFIELM |
| | | | | | TLFGRFSESDMTRVYLRNDASLSRLSKKLFGDWSLIVSALKYYYDAEANPLMGKKATNKYIKEK |
| | | | | | ENWLNKSSNFSIDVINKSLLRYGTINETVNSQFTDDMIFEHFSSFMIEEKNLLNTVAENYMLVSEV |
| | | | | | LSRGSLDKNQNKKKKEIKTIKTFLDTVLDLLHFIKPLSVQYVGAEKDEGFYSDFDVLYDQLSQVI |
| | | | | | PVYNKTRYYLTKKPYSMEKFKMNFKNNTLLDGWDVNKETANKGILLQKEGLFYLAIMNKDHS |
| | | | | | KSFYNIMDTGDTTGYQKMNYKLLPGPNKMLPKVFFGVKNLNFFNPSDEVLRIRNTSSHSKNGNP |
| | | | | | QEGFEKADFSLSDCHSLIDFFKASLNKHKDWKKFAFDFSPTQSYNDISEFYREVENQGYKITYTNI |
| | | | | | SDAYIHELVKEGKIYFFQIYNKDFSPFSKGKPNLHTLYWRALFDEKNLADVVYKLNGQAEVFYR |
| | | | | | KKSIEYSEEKWIQGHHHEQLKDGFAYPIIKDKRFAFDKFQFHVPITMNFKALGAPVINMKVREY |
| | | | | | LKTNPDVKIIGLDRGERHLLYLTLIDQNGNIEEQYSLNEIVNSYNGKVYKKDYQQLLHVKEGDR |
| | | | | | KKAKKNWETIEAIKELKEGYLSHVVHKIVNMMVEHNAIVVMEDLNFGFKRGRFHIEKQIYQKF |
| | | | | | EKMLIDKLNYLVLKDTQDPKTPTGLLNALQLSNKFESFQKLGKQSGFIFYLPAYLTSKIDPTTGF |
| | | | | | VNQLRIKYDSIVKSQAYYRQFDTIVYNNTSDWFEFSFRYVNFANTTPSARKIPWTICTTHHPRYA |
| | | | | | WNINSNVGNGGTEEYNVTKELKKLFDQHKIAYEEGTDLIESIASNTAVDFFKRLNKLLYITASLR |
| | | | | | HNNGKKGKEEHDFILSPVANSEGGFFNSLEADETQPENADANGAYHIALKGLWALQSIRKTDTD |
| | | | | | RLSKLNLAVSNEEWLNFAQAKQYRSQNSS |
| MG29 | 6518 | MG29-195 effector | Protein | Unknown | MFTNDYFSFTLIQTYIDIYNTMLGGKTLADGTKIQGLNELINLYRQKHNIDKRELPNLKPLYKQI |
| effector | | | | | LSDRDGMSWIPEAFECREDLNLAIQTFYNKNIVAFECCDGVVDITEKFLEVLTQTSSYDREKIFIK |
| | | | | | NDLSLTAISHVLCEDYRVIKDALWQKHLQENPKAIKSKDIAGDEERFFGRKNSYFSISEIAKALDL |
| | | | | | IEKPSDLFGYFKTEVEKQSKQVKSSFKEWELDPNNKKLTKEFLDSTLDLQRTLKPLYVRSDIDKD |
| | | | | | IAFYALFDGYFDSLSAIVKLYDKVRNFESKKPYSTEKFKLNFENKGAFLGGWVDSYTDKSDNGT |
| | | | | | QSGGYLFRKKNAIGEYDYYLGISKDTKLFRSHLQNDIDENDISEYERLDYYQLKTASVFGNSYVD |
| | | | | | GSYSEDKIEIKNSIYNFVKSTDLGKELEDYISSKEAKEATPNGMINYIKEKNPTLFEELLEDEEFSK |
| | | | | | INKTVTKKLKETILSLHRVPKSQEYKEASFNLFTEPIEAIEKLSEEKTFVYFSISSREFENALANKD |
| | | | | | KPLLLFKITNKDLSYAETFLNGKRKSRGLDNLHTLYFKALMSGEQAVFDIGTGEVFYRKKSIEYS |
| | | | | | EEKMQKGHHYDKLKEKFSYPIIKDRRYTIDKFKFHLSIIQNYKQPDKYPMFNDTVNSFVKSHHK |
| | | | | | DIKIIGIDRGERHLLYLSLIDGKGAIVEQFSLNTILNTHSNKPIDYHDKLDKKEKARAQARENWGV |
| | | | | | VENIKELKEGYMSHVIHLIAKLIVEHNAIVVLEDLNFGFKRGRFKVEKQVYQKFEKMLIDKLNFL |
| | | | | | VDKQKSPHEIGGVLNALQLTNKFVSFEKLGKQSGFLYYV |
| MG29 | 6519 | MG29-196 effector | Protein | Unknown | MEEIFTEITNKNAFSLQKTLRFELKPMVLNEEENQLQPISESDSYLKNFNSGYLEKLKQIIKHDEE |
| effector | | | | | RAEDYQEIKVYIDELHKQFIDRVLPEIKSLEIDFKKAFEMYELTKKRYAKSSSDKEEEEQSKEKK |
| | | | | | NNLKAWQDFQKEARKKISNFLKKQPEYENLFEKELFSDLIPKSNYSKQLGEKSPNDLAKSFSGFT |
| | | | | | TYFQGFHENRKNIYADEGSTSLAHRIINENLPKFFTNILQYVILNKDHNLLVGQFKENYSDEELTE |
| | | | | | LFNPNSFVSFLNQSGVDRYNEIIEAKKGIETAKSKDGLKQLANKYKQAKQIKNLPNFTPLYDQIL |
| | | | | | GKRGLDQNDESILRAGVTDDKNLLNSLKDFNKNIQPSVFELINICSELNKANAEEIFIMGSSLESLS |
| | | | | | SSVFGDYSVLSRVMKHHYIESRISASRTTEKKLEKDSEAYLKQETYSLQEIQSAIDYYIEKGNELES |
| | | | | | KSLFDYFTACKYNSNHTLSEEIKIAWDNLQPILELEQIDKDRAIPKTQEEQGGKGFQQVEKIKLF |
| | | | | | LDSYMQLLHFAKPLHLVKKRNPVTISKKDEAFYAIFDKNYTNLEAELIPVYNQTRNYLTKKPYSL |
| | | | | | EKFKINFEKGTLLNGWDLNKEKDNLGVLFLKNNNYYLGIMSNNKIFDFQKQNIKKEALSLSGQQ |
| | | | | | DGYHKVIYKYLAGPNKMLPKVFFAKSNLEKFSASEEILRIRNTSSFTKNGEPQPSYKKAEFNLND |
| | | | | | CHAMIDFYKQSLASHEDWSQFGFQFLETSQYSDISQFYEDVAKGGYKISFVNISDNYINEKVKAA |
| | | | | | DLFLFQIYNKDFSEQKKGKDGKPNLHTMYWNAAFRPWLDRSESNVKLNGEAEIFFREHSIERKI |
| | | | | | THRSGESIDKKNPNNSGKSLFSYDLIKDKRFTSDKFFFHVPITINYKNKKERNNKQFNDVVNSVIK |
| | | | | | NNRDVNIIGIDRGERNLLYYTVIDQDGRILEQNSFNEISSQCSTSEKSSTFDYHKKLDEKEDERKQ |
| | | | | | ARKSWGTIENIKELKSGYLSHVIHKLAKLILKYNAVVCLEDLNAGFKRGRMKIEKQVYQKFELA |
| | | | | | LIHKLNCLVLKDREEGEFGSYTNPYQLSSKITSYQDIFSQTGIVFYVNPAYTSKICPKTGFVNFLDL |
| | | | | | RYENLEKAQSLMGSFNSIRFNNHENYFEFDLDYKKIPQTQNKECGEKTQWTVGTYGNERFSYNP |
| | | | | | KTKGYDTYNVTEKLAHLFKKHNIGYEDGSDLREKILASTQDAVSFFKELLFLLRLTMSLRHVNE |
| | | | | | NHDCILSPIKHPELGFFDSRDVKDSIEAEEPRDADANGAYHIAMKGLQIFAEKISSENPKLSIKKED |
| | | | | | WFRFIQNHHESKWKEKSLSPI |
| MG29 | 6520 | MG29-197 effector | Protein | Unknown | GFTFCLNQKGIDTYNLILGGKSLEDGTKIQGLNEFINLYRQQKNLERKSLPNVKVLFKQILSDRE |
| effector | | | | | KKSFVPEAFRSDQHLLNSILDYSNAGIFNWENDDKKKNIVKEIQKFLSNIKSNELDKIYITNDLSLT |
| | | | | | NISSHLLEDWNFIKRSLSYYYDENIGNKAKKEKSPTKYEEEREKWLKQKYFSIELLNNSISLYSTF |
| | | | | | LGEKKFNLTIESYFSKFTSKDEMKKEFNLLERLESTYQELKPLLENEYKKENLKANKGDVEKIK |
| | | | | | NYLDSIKSLQFFMKPLLPKNIQDEKDNDFYNQLEEYLEVINEITPLYNKVRNYLIGKVYSEEKFK |
| | | | | | LNFENSTLLDGWDENKESANLCVMFRDEDKFYLGIMNKEHNKIFSDIPKLKSGETYYEKMVYKL |
| | | | | | LPGPNKMLPKVFFSKSKIDFFKPSKTILKIKETESFIKGDNFKIQDCHALIDFYKDSISKHEDWSKF |
| | | | | | DFQFSKTSSYEDISGFYREVESQGYKITFNKIAKSYVDTLVAEGKLYLFQIYNKDFSKFSKGKPNL |
| | | | | | HTIYFKSLFDKENLKDVVMKLNGEAEIFYRKKSINYDEKTKKEGHHAKELKGKFNYPILKDKRY |
| | | | | | SEDKFQFHFPITLNFKAKEKFNFNQKVREFLKGNKDVNIIGIDRGERHLLYLVMINQEGKILKQV |
| | | | | | SLNSIQTEKKFPEVNYKDKLTEKEIERDKARKSWGTVENIKELKEGYLSQVVHQISKLMVENNAI |
| | | | | | VVLEDLNMGFKRGRQKVERQVYQKFEKMLIDKLNFLIFKENKSKEAGGVLNAYQLTDKFESFD |
| | | | | | SIGKQTGFLFYFPAWNTSKIDPKTGFVNFLHPVYESLDQAKKFISKI |
| MG29 | 6521 | MG29-198 effector | Protein | Unknown | MNENRKNMYSSEDQSTAISYRLMNENLPRFFKSCVSYDKMVAKCPNLNLHIEPTLLKELKELKI |
| effector | | | | | NRIDEVFQPRFFMNLLTQQGIADFNELRGGHTTKDRDKPQGFNEQINLYRQQNPEQAKGVPFFE |
| | | | | | KLYKQILSKEETSSFVLDAFIDDQQLLNALAEFIKLAMNEKGFIKDLQEALQRLGAAHFEKTYVL |
| | | | | | GSGLTNVSKVLFAGDWNLINRALEAQDRALEAQAEKEHPQKGKKISDTLTKKREKFCNQDVFS |
| | | | | | LAELDSLLANYLEQQEKINNEGTAQTQKEWQDTKKNWLVKESITPLSDYFKKSVINAETTATLT |
| | | | | | KQITATKALFKLEALDQNRSTPKTDQDTGGKGFQQVQQIKAMLEAFNNILHSLKPLHLVKGRNP |
| | | | | | IAPNDVDTSFYSVFEETFESYSNNLVPLYNKTRNHLTKKPYQTEKFKLTFSNPTLLDGWDVNKET |
| | | | | | DNLSVILRKDGLYYLAIMHSEHRKIFEQAQEAGVGEDCYEKVNYKLLPGPNKMLPKVFFSKKGI |
| | | | | | EQYAPPAEILRLYKNEEHKKGETFKLDSCHKLIDFFKANISHYKKDANDVGWAVFEFQFSPTKN |
| | | | | | YEDISAFYKEIERQGYKIWFTNIASKYISDAVTAGKLFLFKIYNKDFSLHSKGAKNLHTLYWQGL |
| | | | | | FEQNNLKDVVLKLNGEAELFYRKRSIETGNQIIHLANQPIKNKTKQAKQGSSKFKYDIIKDKRYT |
| | | | | | QDKFQFHVPITLNFKSTNVKQFNHNINTAIQHDESVNIIGIDRGEHHLLYYSVIDQNGTIIEQDTLN |
| | | | | | TIDTGGKGYIVDYKDKLHSKEQERDNARKSWGMIENIKELKAGYLSQVVHKLAKLIIERNAIVC |
| | | | | | LEDLNFGFKRGRFGIEKQVYQKFERALIEKLNYLVFKDTERSDEAGHYLNAYQLTAPFDSFKKL |
| | | | | | GKQSGILYYVPAAYTSKIDPATGFINFLNTRYETIQKAQDFFSKMDKICYNAKENYFEFHFDYNK |
| | | | | | FEVNQDLTAYQTKWIACTHGGDNRYHYNVKQRTLDKINVTDKLESLLQQYKIQYKHGEDIKEV |
| | | | | | IGKIDKADFFKTLLWLLRLTLTLRHSDRNNDEDYILSPVKDKNGVFFDSREQATLPSDKQTLPIDS |
| | | | | | DANGAYHIALKGLWNLQQINDWDSTGKLNLAMSYNDWFKFVRQLKQ |
| MG29 | 6522 | MG29-199 effector | Protein | Unknown | MLSNFTNQYQLSKTLRFELKPIGNTLEHIKQKGLLSQDEQRAENYTVIKEVIDTYHKAFIEESLAS |
| effector | | | | | VVFDNLERFEELYLKSNKDEKEQKEFEKLQENLRKEIVKNFKVHPKWNNLFKKELIKEDLLAFE |
| | | | | | QITDEQKEVVKEFTNFTTYFTGFHENRANMYTDKEQHSSIAYRIVHDNLPTFVNNKKAFESILQK |
| | | | | | YPQLISDAKSSIEEELLGAVFEDMFLLQYFNHLPSQTHIDLYNTMLGGVKRDDLKIQGFNEKINL |
| | | | | | YRQANGLNKKELPNLKPLYKQILSDKDTLSWLPEAFETQEELVGAVESFYQEKILAFECCDGRV |
| | | | | | NLLEKFKEIFSQTQLYDTSKIFIKSDKPLTDISQALFKNYGLLKEALWQKHLDDNPKLQKSKKIE |
| | | | | | ESEEKFFKQKYFTLSSLQEAIEFAKLSANVWNYFQENLDTYIKQIEENHTIWETDKTNTATTKSF |
| | | | | | LDSLINLQRFLKPLNVQTDSDKDIAFYSTFDSYFEALTQIVKLYDKVRNFKTKKPYSLEKFKLNFE |
| | | | | | NSTLLDGWDVNKEPDNTSILLRKNNLYYLAIMDKKYNKLFCNLEKSTQSDVYEKIEYKLLPGAN |
| | | | | | KMLPKVFFSNKNIDYYNPSKKLLENYKDGIHKKGDNFDIDFCHELIDFFKVSIQKHEDWKHFKF |
| | | | | | NFSPTKSYEDLSGFYREVEQQGYKISYKNIDTKLVESWVNDGKLYLFQIYNKDFSPYSKGTPNMH |
| | | | | | TLYWKALFDEQNLANVVYKLNGQAEIFYRKKSIEYTEDKLKKGHHHEELKDKFAYPIIKDRRFA |
| | | | | | FDKFQFHVPITLNFKAEGNENLNQKTIEYIKTNDIKIIGIDRGERHLLYLSLIDLNGRIVEQYSLNQ |
| | | | | | IINSYNGKEYPIDYHEKLAKKEDERALAREEWGVIENIKELKEGYMSHVIHRITTLMVEHNAIVV |
| | | | | | LEDLNFGFKQGRFKVEKQVYQKFEKALIDKLNYLVDKKKTPSDLGGVLNALQLTNKFVSFEKM |
| | | | | | GKQNGFLFYVPAWNTSKIDPVTGFVNLFDTRCSSVEKAKEFFGKFKSIRYNSVKEYFEFEFDYND |
| | | | | | FHNKALDTQTQWTICTYGERIKTFRNKDKNSQWDNETIHLTTAFKNHFGNYQGELKEYILAQD |
| | | | | | KKEFFEQLLDLFKLTLQMRNSITNSEVDYLISPVADKNGNFYDSRKADSSLPKDADANGAYNIAR |
| | | | | | KGLMLVERIKESTDVKKVDFKLTNKEWLQFAQRG |
| MG29 | 6523 | MG29-200 effector | Protein | Unknown | TSLNEIFSLGYFNKILNQTGIDLYNFVLGGKSEKDQKKIKGINEYINLYNQQQTDRKQRIPKLKPL |
| effector | | | | | YKQILSDRNSLSFLAEQFENDNELLEAIEQFRQTGLLHYKTDLKEINVLNEWKTLIENLNDFQRE |
| | | | | | GVYVNNGIVLTDISQQLFGSWEYLKKALVYYYENVLFTDEKEKKKKKYNEAKEKWLKQKQFSI |
| | | | | | AVLEDALAAFKKQETNPEILEKFTEHPVIDYFKKAGNDGNGDLPEKIDATYNTVKDLLNTSLQPN |
| | | | | | EKLVQQKDKTLLIKNYLDSIVNLLHFIKSLKPREELSQKDEAFYGRFDELYEALNVITPLYNKVR |
| | | | | | NHLTKKPYSTEKYKLNFENSTLADGWDLNKEADNTTILLRKEGNYYVAVMDKKHNKIFRDIPAS |
| | | | | | AKGEAVYEKMVYKLLPGANKMLPKVFFSKSRIEEFNPSTELLENYKNETHKKGESFNIQHCYNL |
| | | | | | INFFKSSICQHEDWKHFNFNFSETGTYEDLSGFYREVEHQGYKITFVNISESYIHKLVEEGKLYLF |
| | | | | | QLYNKDFSKYSKGKPNLHTLYWKMLFDETNLKDVVYKLNGEAEVFYRKKSIEDKNKIIHKANQ |
| | | | | | PISNKNPENVKKQSSFQYDITKDHRFTKDKFQFHVPITMNFKAKGILNVNNEANKYLRNNPDTHI |
| | | | | | IGIDRGERHLLYLTLINQQGEIIKQESLNIVANEKQKTDYHKLLEAKEGKRDEARRDWGTIENIK |
| | | | | | ELKEGYLSQVVHKIAEMIVDYNAIVVMEDLNFGFKRGRQKVEK |
| MG29 | 6524 | MG29-201 effector | Protein | Unknown | MHLQLTNQFSVQKTLRFELKPIGKTLEHIEAKGLIKEDEQRAQEYERLKKIIDFYHKDFISEALSS |
| effector | | | | | VRLKGLRCYERLFFDSARDEKVFEKIQTKMRKQMRKAFEEHPNWKYLFKKELLTKVLPNWEH |
| | | | | | PEVTSEDKNIIKNFQKFTTYLTGFHENRKNIYSNEAKHTAIAYRVVHENLPVFLQNKRLFELIKER |
| | | | | | YSKIIEDAKKELNTQDALKGATVEDVFSLDYYQYLMSQTHIDIYNAIIGGIVLDDGTKVQGLNEKI |
| | | | | | NLHRKNGLSKRDLPNMKPLYKQILSDREALSWLPESFEDTQEMSEAIAAYYDANILRFKCCDGE |
| | | | | | VNLLEKFDKLFQESNEYDLNTVYIKNDKSLTDISQAIFGNYSTIKDALWEIIIILRENPRIANASDID |
| | | | | | EKRRKFFDKKKSYFSIAQIEEALKEYGEEISLFDYFKEFGTYGNDEARDLRVEIESAYEAWNNDR |
| | | | | | ENKDKIKALLQSLLNLQHFLKPLYVKDELEKDIAFYAYFDIYYEALSLVIPLFNKVRNFLTKKPYS |
| | | | | | KEKFKLNFENSTLLDGWDVNKERDNLGVLFEKDGLFYLGIMDKKHNKIFQNIKEGGTHNVYRK |
| | | | | | IEYKLLPGPNKMLPKVFFSKRRIDEFAPSESLLAKYKEGTHKKGETFNLDDCHNLIDFFKASIKK |
| | | | | | HPDWREFEFDFTDTKKYQDISEFYKEVELQGYKIAFKNIDAQVIEQFVDEGKLYLFQIYNKDFSP |
| | | | | | YSKGTPNLHTIYWRMVFDDANLKDVVYKLNGEAEIFYRKRSIEYGEKTWKEGHHAKELKDKFN |
| | | | | | YPIIKDRRFALDKFQFHVPITLNFKASNLSNKEHNNLIRQKIKEHTDDIKIIGIDRGERALLYLALI |
| | | | | | DGKGRIVEQYSLNTIVNSYNGKKHKVDYHQKLDAKEKEREEARLNWESIERIKELKEGYMSHVI |
| | | | | | HKIATLIVEHNAIVVLEDLNFGFKRGRFKVEKQVYQKFEKMLIDKLNYLVDKKKEPTELGGALK |
| | | | | | ALQLTNKFISFEKMGKQNGILFYVPAWNTSKIDPTTGFVNLFDAKYYNKEKSREFFSKFKSINYN |
| | | | | | KQEDYFEFSFDYNDFHRKAEGTRTEWTLCSFGERIRTFRNPSKNNQWDDETIDLTKAFKELENG |
| | | | | | QTENLQAYILMQDSVEFFKKLHYLFKLMLQMRNSSSKTGDDYLLSPVKNSSGEFFDSRKSDNSLP |
| | | | | | KDADANGAYNIARKGLMLVQRFKEADDVAKADLTITNKEWLRFAQGIE |
| MG29 | 6525 | MG29-202 effector | Protein | Unknown | NMQRVINEDRKRAEDFSKVKKIMDDFCRYLIQKHLSNVNLDINNLKLFEDSFLELKRKKNAAKK |
| effector | | | | | NDIANARTNLNKIIKELSNELYKKSNLDKIKFKVKEKKDDTESIYFVTALKEWLKENNREDDLAL |
| | | | | | VKKFDKFNSYFYGFFENRKNIFNNTSSRNKSTSILYRAINENLPSFVNNCLKLEELIKLGVDFSEVE |
| | | | | | RNFSKELQGKKLIDFIKLENYGSFLTQEGIDLFNVIVGGKTTENGEKLKGINEIINLESQNRADKN |
| | | | | | IRKLKLDVLKKQILSDSESKSYVPEKLKDDLEFVQNIQNAYMIDDENRIKELYSNLDNYDLNEIYI |
| | | | | | KNDAIITKLSQELFGEFDLLQELIFDYVLKDENFKNKDQKYLKRKKEYFKKQDYFSIFDLESAIK |
| | | | | | YGFNNNDFFNNSEKKNKFLENINNDIDHPILGYFKRFYFENKNLFKEIDDSYKELNNILNSNNLVN |
| | | | | | KKDFLTQGKEEDVRKIKCFLDNCLNLLHFVKILNYKKGDPLDYETLIVDNSFYEKYFEIYDRLVE |
| | | | | | INKIYEKARNYATQKPYSKNKFKLNFDNSKLADGWDRNKETDYFTILLRKNNNFYLAILNRNNK |
| | | | | | KLFKKINYNENEDYYEKMEYKQIPFSKGGLGGFVRKCFNSAKKKGWVCPKDCLNSNGEIIINDN |
| | | | | | DVKNNLIQLIDCYKNFLNIYENSGFKYKDFNFEFKDSKDYSSLDEFFNDVEKKRYFIKFVKVSKKF |
| | | | | | IDDSVDAGKLYLFHIYNKDFSENKCDKNKNSIRNMHTLYWNATFSEENLKNPIFKLNGRAELFFR |
| | | | | | DKSTANNLIIHPKLKPIKNKDPINN |
| MG29 | 6526 | MG29-203 effector | Protein | Unknown | MKSTLDQFSHLYPMSKTLRFELIPQGATTANIESRGFLKKDEERAESYKKMKETIDRFHQDFIEK |
| effector | | | | | AMAHVRLSNLEDFENLYNAPNEEKKEDKYKKQLEKVQERLRKEIAKGFKSGEVKAIFLKIDKK |
| | | | | | DLVTKLLEKWIEENNLEDVHFDPEFKKFTTYFSGFHQNRKNMYTDKAQSTAIAYRLVHENLPKF |
| | | | | | IDNINIFKKVSEIPELKQNLEKLYKEIEEYLGIVSIEEAFELGYFNEVLSQKGIDVYNLILGGRSEKE |
| | | | | | NKKKIQGLNEHINLYNQKQDKKNKIPKLKVLYKQILSDRTSTSFLPDAFEDDDNSTASQKVLAAI |
| | | | | | HQFYHTQLLDYQPSDKAETINVLKSFQGLLADINNFDLDKVYLRNDKSISTIAQKMAGNYGVLR |
| | | | | | DALNYYYENKIDPEFQIKYDKATTDKKRENLDKEKSKFTRQSYISISTLQTALDTYVESFDETHDV |
| | | | | | KQVYSPTCIADYFKDHFKAEPKEGSDKEFDFVSNIEAKLSTIKGLLNTPYPENERLQQDKKKIDAI |
| | | | | | KLFLDSIMEYLHFIKPLALPEDFTLEKDEHFYTLFEEWYEQMQLLIPLYNKVRNYATQKPYSTEK |
| | | | | | FKLNFENSSFLSGWAPDYNTKGGLIIKKQDNFYLCIVEKKLKKEDVEFLKTSPEDHLAHRVIYDF |
| | | | | | QKPDNKNVPRIFIRSKGTSFAPAVQKYDLPVESIIIIIYDEGLYKTDFKKENPAVFKKSLVQLIDYF |
| | | | | | KLGFSRHDSYKHYDFEWKESEEYETIADFYQDVINSCYELRDEAINFDHLHQLIDQGKLYLFQIY |
| | | | | | NKDFSKHSKGKPNLHTMYWKALFEKQNLKDVVYKLNGEAEMFYRKKSIQDKNRVVHKKNIKV |
| | | | | | ARKFYKDDKKTERVPDETVLRLNKFYKGQIQESGLKKEDLKFKDNYSLFHEQGKDIDLIKDKRY |
| | | | | | TVDKFQ |
| MG29 | 6527 | MG29-204 effector | Protein | Unknown | ISDKDGHFYNNFLVYYKSIKEIIPLRHKVQSYVTQKPYSLKKFKVNFDNSYFLTTWPFSYENKGG |
| effector | | | | | VIIRKEDLFYLAIINCSVKDLANYKINDCSIANSAERIIIDTQKPDNKNIPRLFIRSKGDSFAPAVTE |
| | | | | | YDLPINDIIDIYDNGKFKTEHKKLDPEEFKSSLTALIEYFKLGLSRHNSYKHYKYQWKPSDCYNDI |
| | | | | | SEFYNDTVNSCYQIKTEKINFDKILELVSEGKVYLFKIYNKDFSPNSKGQPNLHTMYWRALFDEN |
| | | | | | NLKNVIYKLHGGAEIFFREKSISKENEIVHYANQPIKNKNPHAIKREVILPYDVIKDKRFCIDKFQL |
| | | | | | HIPITLNFKATGKGQLNSDVLQYLKDTPQNQVKVIGIDRGERHLLYLTMIDHEGHIIMQESLNTV |
| | | | | | KSDNYPIETQYHDLLAQKEEDRNKARSNWDSIENIKELKEGYLSQVVHKLAKLIVENNAVLVME |
| | | | | | DLNIGFKRGRFKVEKQVYQKFEKALIDKLNYLVFKDKKVEEEGGLFKALQLTEPYTDFLKYKK |
| | | | | | KQCGFLFYVQAWNTSKIDPTTGFIDMLKPKYKNIPEAQEFFRKFISIKYNSDKDYFEFYFDYRNFP |
| | | | | | RSIDSHKNDWTVCTYGKERYAWNRSLNQGKGDYEIWNVTEKIKELFNIEGIEYRSGRNLTEAIA |
| | | | | | NSESKSLLSMLMKSLSVVLAMRYSSSKDNRDFILSPVANDKGVFYYSEEADQYLPKDADANGAY |
| | | | | | NIARKGLLLLDRIRSSADLKS |
| MG29 | 6528 | MG29-205 effector | Protein | Unknown | DRETSSFIPDQFENDKELLESLGKFINEMAKKGGILENLQNSVRSLKDADMRKTFVKGGVEITRIS |
| effector | | | | | EKIFGNYSILKSAIYHHAETVVYPTPKSGKVSETLEDKRKKYVNKQDVFSITELESMLSIYANHLD |
| | | | | | DDSPDKETIIKYTNVKNIISNHFLSTIDDLINDEAIGFRTAVEEVLSLLSLDRLNKGKQGQEQTHKI |
| | | | | | QKMLDAFLAMSHSVKPLHLVSRRKPIDVPDMDMGFYNNFSKTFEAFDQMVIALYNKTRNHLTK |
| | | | | | KPYSKDKIKVNFENPTLLDGWDINKAKANNSTILRKDGLYYLAIMHSNYNSIFDEIPDVKNHERV |
| | | | | | YEKVNYKLISGINKMLPKVFFSKKGIETFNPPRSILDLYKNSEHKNGDTFNLDSCHRLIDFFKENV |
| | | | | | GKYKVNPTDEFGWDVFNFKFSPTKSYQDISYFYREIEMQAYKIWFADISETYINQCIEEGKLFLFQ |
| | | | | | IYNKDFSPYSTGTPNLHTLYWKALFESENLKDVVAKLNGQAEIFYRKHSIKKDERTIHNANQVIQ |
| | | | | | NKNENNPKKTSSFEYDIIKDKRYTVDKFHFHVPITLNFKAPGVTRENNKINDTLAKSDDTYIIGID |
| | | | | | RGERHLLYYTVLSSKCEIVEQGTLNTVSTNHGYKVDYQKKLDKKEKERDKARKAWTSIENIKEL |
| | | | | | KAGDLSHVIHKLSQLFIKYNAIVCLEDLNFGFKRGRFKVEKQVYQKFEKALIDKLNYLVFKDVK |
| | | | | | FGKPGHYLNAYQLTAPFDSFKKLGKQTGILYYVQASYTSKIDPVTGFINFLYPRYESFLKSKMFF |
| | | | | | ENMNGIRYNAEKDYFEFMIDYSIMTPSRDLSGYKNKWTICTFGDKRFKNFRNANENWESVVVNV |
| | | | | | TAELKQLLEQAGITFQLGQDLRESITQIKDTKFYRKLFKLLQVTLSLRHSKTGTDEDFILSPVADE |
| | | | | | NGKFFDSRKATEKQPKDADANGAFHIALKGLWNIQKIKEWDGTSNLNLAMKNVEWFSFAYNKP |
| | | | | | FLD |
| MG29 | 6529 | MG29-206 effector | Protein | Unknown | MFSYGLYKEEGKKKIQGLNEYINLYNQKQEKKNRIPKLKPLYKQILSDRENISFLPELFEESQDV |
| effector | | | | | LDAIESYYKANLVDYKPDDKDDTENILKELYSLLKGVNSFDTNKIYIRNDKSLTDISKAIFGDWAIT |
| | | | | | DAALEFEYIQDITIPKSGLTKKQEKEKERYLKQPYFTIQEIEQALKAYRNENEVLADFTEGMVGN |
| | | | | | YFYSHFKTKTDSDKEFDFVSNIEAKYSCIKGVLNTKYPKNQKLNQDKVTVNNIKVFLDSLMELLH |
| | | | | | FIKPLALPNDVPFDKDETFYGIIFETYYEQLQLLIPLYNKVRNYATQKAYSKEKFKLNFENSTLLD |
| | | | | | GWDVNKEEANSCVLFQKEGLYYLGIMDKNHNKVFRNLPSTSSKNTYNKINYKLLPGASKMLPK |
| | | | | | VFFSSKNIAYYNPDKEILRIRNHSTHTKGGSPQKGYEKQDFNVKDCRKMIDFFKTSIHKHPDWK |
| | | | | | KFGFEFSETAHYNSIDTFYREVESQGYTITHTAIDNDYIDKLVNEGKLYLFQIYNKYFSPYSKGKP |
| | | | | | NMHTLYWKALFEPDNLNDVVYKLNGQAEVFYRKKSIKEKHKVIHKAQEPIINKNPLAQKKTSVF |
| | | | | | DYDIIKDKRYTVDKFQFHVPITLNFKAVGNEYLNTNVLDYLRDNSDVKIIGLDRGERHLIYLSVID |
| | | | | | QEGNILEQESLNTIINKEHNIHTPYHKLLDKREKERDSSRKEWGTIATIKELKEGYISQVVHRITQ |
| | | | | | LMVKHNAIVVMEDLNFGFKRGRFKVEKQVYQKLEKMLIEKLNYLVFKDKLPNEAGGLYNALQ |
| | | | | | LTNKFKSFKEMGKQNGFLFYVPAWNTSKIDPTTGFVNLFYTRYESVEKAQQFFSSFDSIKYNTKK |
| | | | | | GYFEFAFDYNNFTTRGEGTKTKWTVCTYGDRIITFRNPNANNQWDNKEVNLTQQFEDFLGKHQ |
| | | | | | IVYGDGQCIKDQIVGQVDKTFFKDLLYLIKLTVQMRNSITNSDVDYLISPVTNHSGTFYDSRKASN |
| | | | | | SLPKDADANGAYHIAKKGLQWVQQVQQFEGDDWKNLNLDKTNKGWLRFVQNQ |
| MG29 | 6530 | MG29-207 effector | Protein | Unknown | METKKSIFDEFTNLYSLRKTLRFELIPQGRTLEYINKKGLISKDEQLAQSYQRMKKTIDGFHKYFI |
| effector | | | | | DLALSKTKLTRLKEFEELYSADAEKKKEESFKKEFGKVKEILRKEIVSSFESGEAGKTFSKLTKA |
| | | | | | DLLKGKNKNSDETEDGDGSDENTGNKHTELKGLIEEWMEKQPNKDEIYFDKEFKKFTTYFSGF |
| | | | | | HTNRKNMYSDEDNSTAIAYRLIHENLPKFIDNIRTFGIIKNKIGLYEDCNKLHNELSKYLNIENIDE |
| | | | | | VFKLDHYNTVLTQKQIDGYNLIISGISVKEGKKIQGLNEYINLYNQKQTDKKNKVPKIKLLYKQI |
| | | | | | LSDKESVSFLPDNFEDDSNRTASQKVLDAINEYYHSNIITFQQIGASHPENVLIKLKELLQELKEYD |
| | | | | | LNKIYIKNDSNLTLISNKLFGNRNVFDLALNYYYEFVERPNFNNEYQKAKESKREKLNKDKEKFV |
| | | | | | KASNVSFLLLQSALNEYVQRLDSSHEIKKKYSDSCITNWFIDYFKTESKDCVEKELIKNVEEKYNA |
| | | | | | IKGILNIKYPSDKKLYQEKDNISVIKTFLDSLMEFLHFVKPIVLDKDSSLEKDNSFYAQFDLYYEEL |
| | | | | | NKIIPLYNKVRNYATQKSYNVGKIKLNFENGTLAAGWDKNKETDNTTVIFRKDSLYYLGILDVN |
| | | | | | NKQVFKKQFLKSNGESVFEKMEYKLLPGANKMLPKVFFSKKNLKKFNPTKDLLIKYKNGIHKK |
| | | | | | GDNFNLDFCHELIDFFKKSISIHNDWKHFNHKFSPTFEYKDISDFYREVEQQGYKITFQNIPEAYIS |
| | | | | | SLVSEGKLYLFQIYNKDFSQNKKAKGTDNLHTLYWKALFDEGNLKNVVYKLNGEAELFYRKRS |
| | | | | | LNYSEEIMKKGHHSEMLKNKFNYPIISNKRFASDKFQFHVPITLNFKSSGFKNINLSVLNFLKNNP |
| | | | | | KINIIGIDRGERHLIYITIIDQEGNIKLQKSFNELFYNVKGESQSRKVDYREKLDKREDERKEARE |
| | | | | | KWGVIESIKELKQGYLSIVVHEIVKLMVKYNAILVMEDLNFGFKRGRFKIEKQVYQKLEKALID |
| | | | | | KLNYLVFKDNDAFEFGGIYKALQLTSRFESFKKLGKQSGFLFYVPAWNTSKIDPNTGFVNLFKTT |
| | | | | | YESIEKAKEFFNSFDAIRFNKTNRYFEFVVEDYTKFNIKAEGTRNNWIICTKGDRIHIFKNKDKKD |
| | | | | | NWDSKIVNLTDRFKSIFKEQNIPFMDEENLIEEIISKDDISFFKNLLDLFRQTLQMRNSIPHKEMDY |
| | | | | | LVSPIADRNGKFYDSREATDEEPKNADANGAYNIARKGIILLNKIKDLENLKKPNLTISEKEWLKF |
| | | | | | AQKIGVK |
| MG29 | 6531 | MG29-208 effector | Protein | Unknown | MNQNKSIWNQFTNMYSISKTLRFELIPQGRTLENVKNKGLIQEDKEREKEFNKVKKVMDRYYK |
| effector | | | | | YFIEKALAQVSIDTEDLRKFQEYYEDLKKDRNNESLKKSYAEVQKKIRKSIYEKIKNMNEFDHIF |
| | | | | | KREFIQKILPKWLKENNEHDDACLVANFNKWATYFTGFFKNRKNVFSADEIATSIIFRIVNDNLP |
| | | | | | KYIDNLSRFNKLKSYNGASFEKLENDFKEELDERNLEDFFALKNFNHCLNQKGIDHYNLIVGGKT |
| | | | | | LEDGTKIKGLNEYINELSQKAENGREVRKLKMMPLFKQILSDRESASFLPEKFENDREVINSINEY |
| | | | | | YEHVLAQFPRLKKLLSEMPKKDINQIYMRNDRSLSEISQEIFKDYQIIKSGIREYGLKTLGIKDTKI |
| | | | | | DEWLKKQSYFSIAEIERGLTLLELENFSQENPILSYLIGLHKEGKDLIKEISEAAEAFKKIEGSEKK |
| | | | | | LLLKKDKEVEVIKGFLDSLKNLVHFMKPLYYDSRHNIDEKGKQALELDMDFYFHFNQIYEDLSK |
| | | | | | IIPLYNKVRNYVTQKPFSTKKFKLNFQCSSLLTGWFSDFRTNAGLILKKNNAYYLAIVPKKLDKV |
| | | | | | DRESLSILDNSESIKVLKYDFQKPDYKNIPRLFVRSKGSNFATAVKKYSLPINEIIEIYDNGYYKTE |
| | | | | | YREKDPQKFRESLKKLIDYFKEGFQKHESYKHYTFIWRDSEEYNSINEFYEDVINSCYSLSFYKVN |
| | | | | | YGYLNELVNQGKLYLFRI |
| MG29 | 6532 | MG29-209 effector | Protein | Unknown | MKNQHLFSGFTNQYAVSKTLKFALKPTDKTKEHINNKNDKGKNLLDVDKELAEKYKQAKKIID |
| effector | | | | | EYHKSFIHEKLSNFSFGKKELQDFNNAYEDLKKDKKNDSLKKDLILKQDELRKQVAQELQKNN |
| | | | | | YLYKKKGGKAKFVKQRLPEWAVENANQIKKNHENIDNPEAIIKDFKNWTNYFGGFNDNRKNIY |
| | | | | | TEKSHSTSIGYRLIHENLPKFLENIKRYKEAKKLDIDFSEVEQKFDVNPDNIFTLNYFNQCLTQEDI |
| | | | | | EQYNLIRGGQSKVDNKKERGINEIINLYSHELQSKLTDAQDEDKKELKAQIKQVRSCKLEELYK |
| | | | | | QILSNRSQISFPLENIKNDSELCQQIEHLFYLNNRGSLIGKQEETGEIAEVIFDISEAIKKAIQAISDA |
| | | | | | DPEQLYIKNKSSRAITDISQHLFGDWNLITKSLNYYAEQTIFSAPKDKKETAKQQEVREKKLENW |
| | | | | | MKNIPYFSFAEIHAALEYYFKQYSNKELQNEKTVKNEQSEQGITKEMKETALKKPLFTYFKELII |
| | | | | | TKKNEKDNTFEKIFLSEQIKTTYPIAKKVFEQYKDVKKELLKSKKDEVHNIKAYLDSLMDLLHFL |
| | | | | | KPLYVQFSKKEEKKQVEIFEKDNSFYGDFDVLFKVLNQIIPLYNQTRNYLTKKPFSIEKYKLNFE |
| | | | | | NSQLADGWSKNKETNNTCVLLMKNEQYYLGVMNKKHNKLFSDELPNKGSCYQKIIYKQVSDA |
| | | | | | AKDIHTLVNIEGKVSRFTKGLNEKRSKYCQEIQNIKSQESYKGKGLIKSDLYKFIDYYKKCAKSY |
| | | | | | WNWCDFEFEKTEKYQNIKEFTDDINRQGYKITFCDISESYINQCVNEGKLYLFHIYSKDFSIHSKG |
| | | | | | RKNLQTLYWKALFDENNLKDVVYKLNGEAELFFRKASIQYSDDIWQKGHHYEELKGKFQKEG |
| | | | | | KHLPIIKDRRYAKDTYLFHVPITCNFKAEGVTKFNDYVNKEFLQKNSNVNILGIDRGERHLAYYT |
| | | | | | LINQNNEILKDKDGKYLQGSFNNPTGRKDYHELLNKREQERDKARKSWGTIEKIKDIKEGYLSQ |
| | | | | | VVHKIATLMIEHNAIVVFEDLNFGFKKGRFKVEKQVYQKFEKMLIDKLNYLVFKDKSHDEPGGL |
| | | | | | LNAFQLTAPFESFQKLGKQTGFIFYVPAYHTSKICPATGFVNLLYPRYETVKKSQEFFDKFNKICF |
| | | | | | NKEENYFEFHFNYAKFTDKAKGSEQNWIVCSYGKRLENFKNPQQNNQWDTREIDLTQDLQKLF |
| | | | | | QKHNIQFDVDVCIINSICQQNDRQFFKDLIRLLRLTLQMRNSRINSDEDWMISPVKKNQDKFFDS |
| | | | | | RKVDDSMPQNADANGAYHIALKGLWCLQQINETDNTKNLKLAISNKEWLAFAQNK |
| MG29 | 6533 | MG29-210 effector | Protein | Unknown | SIIRAAIYNYAETVMYPTPKTGKVSDVLEEKRKKYANNEDIFSIAELESMLTSYREQLEDEHPDKE |
| effector | | | | | IITHCENSEHPIRTYFLNIIDNVKNDKDIELGKSIENVLPLLSLENLNKGKAGQTQTHLIQKMLDA |
| | | | | | FLAVTHAVKPLHLVKGRKPIEVPDIDMGFYADFSSAFEIYQQMIIGLYNKTRNHLTKKPFSTDKI |
| | | | | | KINFENPTLLDGWDANKENDNSGILFKKDGNYFLGIMHPKHKNIFNYIKGINDIESEKRSLSKDEL |
| | | | | | FDKIVDGNSDHYQKIVYKLLPGVNKMLPKVFFSGRRIDYFAPSTEVLKIRNTASHSKNGKPQKGF |
| | | | | | EKADFNLKDCHTIIDFFKQSLEKHPEWKEFEFDFSPTSSYEDLSGFYREVEHQGYKMDFHPIKKS |
| | | | | | YIDQCIEDGKLFLFQIYNKDFSPYSKGKPNLHTLYWKALFDPENLKNVVAKLNGQAEIFYRKHSI |
| | | | | | IKDDRTIHRSNKSLQNKNENNPKKTSLFEYDIIKDKRYTVDKFQFHVPITLNFKAEGVTRENDKIN |
| | | | | | RELSKSDDTHIIGIDRGERHLLYYSVINHKGEIVEQETLNTISTDQGFAVDYQQKLDKKEKARDK |
| | | | | | ARKSWSTVENIKELKAGYLSHVVHKLALLIVKYDAIICLEDLNFGFKRGRFKVEKQVYQKFEKA |
| | | | | | LIDKLNYLVFKDAKPNEPGHVLNALQLTAPFDSFKKLGKQTGILYYVQAAYTSKIDPVTGFINFL |
| | | | | | YPKYESLIKSKTFFESMEGIRYNPNKDYFEFSFDYRKMTP |
| MG29 | 6534 | MG29-211 effector | Protein | Unknown | MIKKTLPEWLEEKNPEIEGVEDPQAIIKTFEKWTIYFRGFNENRQNFYTDQAKSTSISYRLIHENL |
| effector | | | | | PRFIDNIKRYKDAKALNVDFSEVEKSFAAKLDEVFSLEYYNHCLTQKGIDVYNQITGGRSEKGNV |
| | | | | | KKQGVNEKINLHAQKLKSKISNASDEGKKELEEKSKKVRSCKLEKLHKQILSDSSERSFRLDEID |
| | | | | | SDSSLCEAIVSTFEIDDDENLIGKTKDGDFNITKNVEESLRSLKNSNPEKVYVNNRSITVISQNLFKS |
| | | | | | WNTINNCLEYYAENKRYPVPEGKKETKTLLDQREKWLKQSYFSFADIHKALETYFEQYAEDEF |
| | | | | | KNEQKHEEINDRLNIKEQKEIAKSKPLFGYFEGLIIRKKDEKNKIFEAKHLLEEIQRTFKEVPTIL |
| | | | | | EEYRNVDEEKLKNEKGKVQKVKLYLDALMDLQNFLKPLFIQLNKKEEEKAIELYEKDTGFYEG |
| | | | | | FDSLFEVVKQIVPLYNRTRNYLTKKPYSVEKYKLNFENSTLADGWDKNKEKDNTCVLLMKDGQ |
| | | | | | YFLGIMDMDKKHKKIFEGDLPDEGQCYKKIIYKQISDASKDIQNLICINGKFQRKTKKLDELKEK |
| | | | | | HIPDIAKIRSCQSYKTTNKNFNKEDLITFINYYKKAATKYWDWCDFSFRESDKYDSFKDFTDHVN |
| | | | | | SQGYKIDFQNISETYIDDCVKKGKLYLFQIYSKDFSPKSRGKPNLQTLYWKALFDEKNRRDIIYK |
| | | | | | LNGKAELFHRKKSIEYSQLIWDKGHHAGKIRQKYPIIKDRRYAQDTYLFHVSIQCNFKAGAKKP |
| | | | | | DDFNREVRDSLKDNSEVNIIGIDRGERHLAYYTIINQKGEILKNANGEYLQSSLNKPLGEKDYQAL |
| | | | | | LHKREKERDDARKSWGTIERIKDLKEGYLSQVVHKIAKLMVEHKAVVVFEDLNFGFKRERFKV |
| | | | | | EKQVYQKLEKMLIDKLNYLVFKDKEPDETGGLLNALQLTAPVGSFRELGKQTGFVFYVPAYHT |
| | | | | | SKVCPATGFVNLLYPKYETVKKAREFFKKENKICFNKNCFEFHFDYEKFTNKAEGSRQDWVLCS |
| | | | | | YGVRLENFKNKQNNQWDTREINLNDEIEKLFKDNSIDCNNGQCLIEKIVQQDKSEFFKSLLRLLR |
| | | | | | LTLQMRNSKIGTDEDWLISPVKGSKGNFFDSRKADVSMPQNADANGAYHIALKGLLMLEQLRE |
| | | | | | REDVETFKPNLSNEKWYEFLAKRYENKSGN |
| MG29 | 6535 | MG29-212 effector | Protein | Unknown | KNFYNLYEKIKEIKRGKKERESELKKLNKEFSKKQKAFRKDLYDQIKRQENFQDLFKKELLTKV |
| effector | | | | | LIKWLNEKYADDPKEKENLYLVKKFTNWTTYFTGFNENRKNIYSDKEIHTSIIHRIVHVNLLKFL |
| | | | | | DNLSKFRFLKKQYLTDNKKLESVFGNIEDDLKEEFDGKKLDEIFSLSYFNFCLSQKGIDKYNTIIG |
| | | | | | GKKLENEIRKRQGVNESINLFSQNPEKYIRGEVQDRNTVKKGIKRLKMESLFKQILSDRESSSFVL |
| | | | | | DKFEDDKDVINSLNRFYKGICGGNAKEETNVFEQIREILSKLDEYYLENIYVKNKNSIRHLSKYLY |
| | | | | | GFYDSLTDALKCYAEKKLFPFKKKEIPTKKELKQIEDWIKKTPCFSIKQLQESIELYSKFFDKRAE |
| | | | | | GTVLSYFSDFKKHSGEQKKDILEEVQKKYEEVKNILDEDQIDGKKILIEKEDYIERIKAFLDSIFDV |
| | | | | | FNFIKPFQVSAEDKDGNFYSEFETIFERLEKIIPLYNKVRNYITQKPYSTEKFKLNFENSTLAKGW |
| | | | | | DLNKEKKNTTVLFKKHDLFYVGVMDKDHRKILENPPFSKENDSYYEKIVYKLLPGANKMLPKV |
| | | | | | FFSKKHLDYYSPSEEIIRIRNHSTYTKNGDPQEGYEKKDFILNDCHKMIDFFKKQIEKHPEWRNY |
| | | | | | NFKFSKTTSYGYIDEFYREVEIQGYKIDFKRIDEEYIEELINEGKLYLFKIWNKDFSKYSKGKKNL |
| | | | | | HTLYWNAVFDEKNLNDVVYKLN |
| MG29 | 6536 | MG29-213 effector | Protein | Unknown | MVPWKQYVVYGLKESGKRGIRKIGGKMAKMKSTWDEFTNMYSLSKTLRFELKPVGKTLEHIK |
| effector | | | | | EKRLISQDKERAENYKKMKKTIDEFHKDFIEIAMSQVKLTKLKEFANLYNASPERKKEDSFKKA |
| | | | | | FEKVRAELRKDIAKGFKTGEAKEIFSKIDKKDLITKLLEDWMKKRQENLYFDENFKTFTTYFGG |
| | | | | | FHENRKNMYTDKDQSTAIAYRLIHENLPKFLNNINTFKKLRDKPELHEECKILFKEIKDYLDISSI |
| | | | | | DDVFKLDYYNKVLTQKQIGVYNLIIGGRTAEEGKKKIQGMNEYINLYNQKQDKKDRIPKLKPLY |
| | | | | | KQILSDRESVSFLPEKFEDDPDSTASQKVLDAIREYYETNLIDFKPDGKDETENVLKKVKELLADI |
| | | | | | KAYDLSRIYVRNGKAISDISKALFGDWSIIKSALEFTYTRTIKVGKKGLSKKQEKDRERYLKQSH |
| | | | | | FSIDEIEKALFKYKNENEALKDLRENTTPIAVYFHTNFKAEKKEESDKEFDLIANIEAKYSCVKGI |
| | | | | | LNTDYPKDKRLHQEKKTIDDIKAFLDSLMEFLHFVKPLSLPSDNPLEKDEIFYSQFQLWYDQLRL |
| | | | | | LIPLYNKVRNYATQKPYSIEKFKLNFENSTLLAGWDLNKEADNTSVLFRKNGLYYLGIMDKKHN |
| | | | | | RVFKNIPDANPGESAYEKMVYKLLPGANKMLPKVFFSNIRNEMFAPPEELKKKYEAETHKKSGK |
| | | | | | FNIEDCHTLIDFFKESIAKHEDWKHFGFQFSDTSSYEDLSGFYREVEHQGYKITFQNVPESYINRL |
| | | | | | VDEGKLFLFKIWNKDFSTYNKGKPNLHTMYWKALFDPKNLKDVIYKLNGRAEMFYRRKSISKD |
| | | | | | RKFVHKANKSIENKNPNNTKKESVFEYEIIKDKRYTMDKFQFHVPITLNFKAKRNEHINASVLEY |
| MG29 | 6537 | MG29-214 effector | Protein | Unknown | MVKNLYSEFINIYNISKTLRFELIPQGKTEDNIQKKGLIDNDLERDNEFLEIKKVMDKYFKYFIEV |
| effector | | | | | SLDKIEINKLDLMEYNRIYCELKKDKTNENLKEEYSKIQNKLRKYIYSEVKNKPNYNELFKKDFV |
| | | | | | QKILPAWLSGISEDLDNKIKDELIKVKKFSDWITYFQGFFENRKNIFTDAEIPTSMIYRIVNDNLPK |
| | | | | | FLDNLSKYEELKKYENKGFDFNYINNHFKSELNHLDINNFFTLSNFNVCLNQKGINNFNTIIGGKS |
| | | | | | LENGEKIKGLNELINLYSQQNNEEKSIKKLKLIPLFKQILSDRESFSFILEKFKDDKDVINTINTFYV |
| | | | | | ELNNQNVLLNLKNEILSLGDKDLNLIYIKNDKNLPLISQRLFGDWEKIHQGLRLYAKYNLPNLKG |
| | | | | | DKKIESYINNTSYFSIYELEKAIELLNITNEITNENLKVYLEKINGNIKNPICGYLSLFKNKDENLFE |
| | | | | | NINNNYLKFQKVLNKNFKENQKKLLTLDFESDSEIIKIFLDSILSVFHFMSSLNIRLNKKDEDKGQ |
| | | | | | EAFEIDTEFYLKENENYEKLKEIINIYNKTRNYITQKPFSTKKFKLNFENSTLLAGWDKNKEPDNF |
| | | | | | SVIFRKENNYYLGIMAKGNNKIFSKIENLNINSNYFEKMEYKLLPGPEKMLPKVFFSSKSIKFFNP |
| | | | | | SSEILAIRNYSTHTKGGNSQKDFDKKDFNLNDCHKMIDFFKESLNKHEEWRNYNFKFEETNTYK |
| | | | | | DISEFYTDISNQAYSINFIKIPQKTIYDLVEDGKLYLFQIYNKDFSSFSKGKKNLHTIYWEELFSKD |
| | | | | | NLNDIVYKLNGQAEIFFRKKSIDAKITHPKNQEILNKDPIKNKNKSNFSYDIIKDKKYSSDKYLFH |
| | | | | | CPITLNFKCKDKNKELNKKINAVISKNSNEINVLSIDRGERHLAYYTLLNSNGEILKQDSFNIITDE |
| | | | | | FSRNVNYHTKLDKLESSRSDARKNWKNIENIKELKEGYLSQVIHNIAKIAIEHNAIIIFEDLNFGFK |
| | | | | | RGRFKIEKQVYQKFEKMLIEKFNYLMFKDIDKNQVGGSLKAYQLTPKFESFKKMGKQTGIIYYV |
| | | | | | DANFTSKICPKTGFINLLYPKFENIEKTKDFISKFKYIKYNKSENLFEFNFNYSNFKLAKEESKVIQ |
| | | | | | DNWSIYSNGKKLVSFKNSDKNNNWDIKEVEVNLELKKLFEEFNIDFKSGENLISKITKQTEQKFF |
| | | | | | KSLIYYLRLILQLRNSKINSDEDYILSCVKDKEGIFFDSRNASKNEPKNADANGAYNIGIKGLMLV |
| | | | | | EKIKKLKPNEKLDPRIIRDDFINFVIKKNK |
| MG29 | 6538 | MG29-215 effector | Protein | Unknown | MALIADEFLGQYSLSKTLKFELVPQGKTKDLINNLDDSILAIDAKRAAEYKNVKKILDDYYRFFIE |
| effector | | | | | QVLEKNILDEKDVKEAHVAFQQRAKDSKAFEKTQDNMRKKIAKALKDGRSGSQLDAYEKLFKN |
| | | | | | DDKSGLYKWLNYRKDRKELTEELYESYKKSLQQFDKFTTYFTGYKDNRENLFSAEEKSSAISYRI |
| | | | | | VNENMVRFFENCQRFEDIQKKHEGLYEQLKDNQAIFQFNKFTELLGQSKIDEYNRMIGFSIENSD |
| | | | | | TKGINSLINEYRQKNHIKNRELPMMVQLYKQLLSDREKSFVIDEITSDEEMEEKATECCLEVREI |
| | | | | | EKKIALLVKEYVTGDNTGRIYLRGSKLTDLSQNIFGQWDIINKALQMKLETLATKKHKEEFDKR |
| | | | | | SKKAININELNDILQEYFIGLDSGEYKIMQEKPALSELIIENIPVVEYSPVLNGLGFGTKEERINKIK |
| | | | | | GVFDQIISMLHYYKIFYLYEGNKQLEVAEKDAFFYSEFDGLYNDLSLATKVYDHVRNYVTKKPY |
| | | | | | SENKIKVNFNAPTLLNGWDINKEESNLSVLLEKNGLYYLAIMDINHRRCFDLKDIEVAKAALCDV |
| | | | | | DKPCFNKIEYKQVTGANKMLPKVFFAESNVDYYAPSSEIMTIREKGLYKKDANNIKAMWQWIDF |
| | | | | | CKQSTEKHPEWNKYFKFNFKPTKNYMDVNGFYRDFDNQAYAIKKVRISEKYISDLVAEGCLYLF |
| | | | | | QIYNKDFSQYSKGKQNLHTMYWRMLFDSQNLKNIELNANAKIFKLNGEAEIFFRRQSLEKNITH |
| | | | | | AKDMPIENKNPHNPKKQSTFEYDLIKDKRYTENKLFFHCPITINFRAASLPVQFNKKVNKFVANN |
| | | | | | PDINIIGIDRGERHLLYFTIINQKGEILKQGSLNHIKDNYISNGKEVPVDTDYHELLDRKEKERDA |
| | | | | | ARRNWTTIENIKELKSGYLSQVVHQLAELMIEYNAIVVLENLNAGFKNGRVKVEKQVYQNFEKA |
| | | | | | LINKLNYLVFKDCSLNQPGGVLKGYQLTAPFDSFRSLGSQSGFLYYVYPSYTSHICPKTGFVDLL |
| | | | | | HPKYQSVAEAQRFFERFEFIRFNQDEGYFEFGLDYDRFGKKMNKSKWIVCTYGEERYGFDGKD |
| | | | | | MTAKKYNVTDEISALLDKVKIVYGGGRDIKNDISTQDDKAFFKSLLYFLCLTMQMRNTNGGTND |
| | | | | | DNDYILSPVQDKKGNFFDSREANDTEPKNADANGAYHIALKGLKLISSIDEEGKIVLKKTETQDW |
| | | | | | FNFAQEKSYLK |
| MG29 | 6539 | MG29-216 effector | Protein | Unknown | IMEDFVRKYALSKTLRFELKPVGETAEKINDFKNQAIAAVVAQDKQRAEDYKAIKKIIDDYHRFF |
| effector | | | | | TEQVLGQKILTDDDILKAFKAYDVFRGSKDDTKKKQYQTVQKKLRDKIAKAFKSRFSEYNLFDS |
| | | | | | GLLNEKGSGKNKTKGKLWQWLKKRYDNREITEKQFEEAEKLIKNFDRFSTYFTGFNQNRQNIY |
| | | | | | SAEEHQTAISYRIVSENMTKHFNNCLNFQAIQRRRELSGLKQQLKSCKNVFTPAYFKKCLSQSGI |
| | | | | | NSYNEKIGYKSDDINAKGVNQLINEYRQKNKIDNRQLPFMTVLYKQILSDTEERFKIDVFEDDKQ |
| | | | | | MLKTIDQFYLKIENDLDNLKTCLKKYLTKENLCNIYIKNDVSLTGISQNLENDWALMKKALEEY |
| | | | | | AATFGLNKAKKEKWLKQDCLSMEDIQNALDNYSGSLEEKKNHRTLAEYFTDFKNGEQDLIEKV |
| | | | | | QQAYENAGAVLTLEKLDKDRHEPKNGREGGRGFLQIEKIKNLLDSIMEIVHFVKPLHLVKGGSII |
| | | | | | EVGDKYTDFYDTFDGLYKNISGVIAIYNKTRNYVTKKPYSTKQFKINFENSTLLSGWDVNKETAN |
| | | | | | LSVLLFKDNKYYLAIMKKDAGEIFNYRSAPNDSEKKRKAKERLRKQIIVDRGEHYKKMVYKQIG |
| | | | | | QIHTQLPRIVFAKRNKNIFNPSSKIVKLYQSDSFKKGDGFSINDLHLLVDFYKSCLKKYPGWQIFN |
| | | | | | FDLSPTKSYENISQFYKEVAEQGYNISFNKIKANYIDEKIKDGELYLFEIYSKDFSPYSKGRPNLHT |
| | | | | | SYWKLLFSPENLNDLVLKLNGQAEIFCRPASLKKNETTVHRANQPIDNKNPLNPKKQSKFGYDII |
| | | | | | KDRRFTRDKFFFHCPVTLNFKSPVTGRFNDDVNMYLKNNSDINIIGIDRGERNLLYYSIINQKGQI |
| | | | | | LEQGDFNIVSNSYKNGGKIETDYRGLLDEREKQRDAARKSWSAIENIKELKSGYLSHIVHKIALL |
| | | | | | MIKHNAIVVLEDLSIGFKRTRFFREKQVYQKFEKTLIDKLNYLVFKNAEAGGAGHYLKAYQLTS |
| | | | | | PFVSFQKLGKQSGFLFYVPSFYTSKTDPVTGFVNLLNTRYASVEKSKRFFEKFYSISYNKSNDYFE |
| | | | | | FSFNYA |
| MG29 | 6540 | MG29-217 effector | Protein | Unknown | MKNILKPFTNKYSLSKTLRFELKPVGATLTNIEKKGLVSEDENLAVSYKKLKKVIDEYHKDFIGL |
| effector | | | | | ALKDLKLNILDDYSDLYYKTIKDEIDKKRFIELQLNLRKQIVDSFSKNASDEIKNKFDRLFKKELI |
| | | | | | QIDLIEWLKSKNDFETIELVEKFKTFTTYFNGFNENRKNMYSVDEHSTAIAYRLIHENLPKFLDNL |
| | | | | | KAYRFIKKSYLDFDFNKIEKELELISVSFDSIFDVNGFNQTLTQNGIDFYNTMLGGLTEGHGKKKI |
| | | | | | KGLNEFINLYKQEKIILKSKEIPSLKVLFKQILSDRESVSFLQDEFIDDSDVLNSIEVFYREEIKEKVI |
| | | | | | DGNTINILETIDSVLKEIESFDTSKIYLRNDTSLTDISQRLYGSWSVVKNALSHYFEEIVKPLNGKK |
| | | | | | RTEKYDKELEQWLGKQNQQFSIKFLQDVCTSYFSSQDEKPLNVNGKEWLEYFKNTGSISNDVNS |
| | | | | | ISFIKRIETAYSAIESFLNVELNSSNRKLVQEQVKVDLLKLFLDEIVTFLHFIKPITLKDSSIEKDDVF |
| | | | | | YSVLEGLYNQLDFVTPLYNKTRNYLTKKAYSLEKVKLNFQNAQLLNGWDVNKETDNTSILFRK |
| | | | | | EGLYYLCVMDKKHINKVFKSPNDFPKNEEEYYEKVNYKLLPGANKMLPKVFFSNKSIEYYAPSFE |
| | | | | | LLEKYKNETHKKGETFNLNDCHDLIDFFKESINKHPDWKNFNYQFSETSSYEDLSGFYREVEHQ |
| | | | | | GYKITFQNIATSYIDDLINEGKIYLFQIYNKDFSPFSKGKPNMHTLYWRALFDENNLKDVIYKLNG |
| | | | | | EAEIFYRKKSLEYSDDIWLKGHHANDLKGKFDYPIVKDKRFALDSFHFHVPITMNFKANEGNNF |
| | | | | | NGQVNEFLKNNKDINIIGIDRGERHLLYLTLINQRGEIIIQKSLNTITNKVKDELVSVDYHKRLDD |
| | | | | | REKNRNNARKTWGTIETIKELKEGYLSLVIHEVAKMMVENNAVVVLEDLNFGFKRGRQKVEKQ |
| | | | | | VYQKFEKMLIDKLNYLIFKDRKDDEIGGVFNALQLTSKFESFQKLGKQSGFLFYIPAALTSKIDPA |
| | | | | | TGFVNFMDTKYYSVEKSKEFFGKFSNIQYNIDKDYFEFEFDYNSFTTKAEGTKTKWKVCTSGDE |
| | | | | | RWRYNPTTKNSERVNVTAELKTLFEKNQIIFKNGNDLKSYIIEKDEKGIYSTLLHLLGLTLSLRHS |
| | | | | | ESGTENDFILSPVFNDSEVFFDSRKATEKSPKDSDANGAYHIALKGLWALRQIDSCDDWKKLKLA |
| | | | | | ISNKEWLDFVQKRPFEK |
| MG29 | 6541 | MG29-218 effector | Protein | Unknown | MKLNNFTNQFPLSKTLRFELIPQGNTLEFINKKGLLEKDNNRADSYKKLKKITDEYHKLFISESIN |
| effector | | | | | GFRLFDLDTYAELYQKKEKDDKDKLTFEKVKGVLRKQISDTFKVQNKYQNLFAKELIKEDLLAF |
| | | | | | VGIEDKPLVNEFKDFTTYFTGFHENRKNMYVADEKATSIAFRLINENLPKFIDNLNISSQILGRIN |
| | | | | | GLAEEFKLALLEMEELVQDTKLHEIFNIDYFNETLTQRGIELYNTVIGGKVSEDGNKKIKGFNEY |
| | | | | | INLYNQLQQDKKNRLPKFKQLYKQILSDRNSTSFVIDNFESDSQLLESIEQFYQSGICHFESDGNSI |
| | | | | | NILIAIQALLSSLANFDLSKIFISNGSSLTEISQKLFGDWSAIGAALGEYYDKEYPSKPKERPDKFQE |
| | | | | | RKEHWLSKTSYFDIATIQLSLDCYQNENVKEKNKPNVLIDFLGTMGTDSATKLNLINIVEQNYAII |
| | | | | | KDLLNNNYPEEEKLGSSKEQVAQLKIFLDGLMNVLHFIKPLNIKSEGLEKDETFYSVFIQLYEQLS |
| | | | | | HVIPLYNKVRNYLTQKPYSITKVKLNFENSTLLNGWDVNKESDNSCVILRKNGLYYLGVMDKLN |
| | | | | | NKIFERNVPKCLVESSAFEKMNYKLLPGANKMLPKVFLSKKGLETYSPSFEIVENYESESHKKGE |
| | | | | | TFNIDHMRGLIDYFKNSISRHPDWKEFKHKFSPTESYIDLSGFYREVEQQGYKITYTNIDESYINH |
| | | | | | LIDDGKLYLFQVYNKDFSPFSKGTPNMHTLYWKMLFDKNNLTDTVYKLNGEAEVFFRKSSISQK |
| | | | | | NRVVHKANESIKSKNDLNLKRQSNFNYDIVKDKRFTLDKFQFHVPITMNFKATGNDNINQNVNQ |
| | | | | | FLKNNQDVNIIGLDRGERHLIYLTLINQKGQILKQESLNVISNEKQEVNYKEILQKKEGDRTQAR |
| | | | | | KDWNTIENIKEIKEGYLSQVVHKIALMMVEQNAIVVMEDLNQGFIRGRQKVERQVYQKLEKML |
| | | | | | IDKLNYLVFKNKSPGESGGLLKALQLTSKFESFKSMGKQSGFLFYVPAWNTSKIDPTTGFVDFLK |
| | | | | | PKYENIDKAKAFFKKFDTISFNNEKNYFEFKFDYKDFTTKAEGTQTNWTVCSHGKERYTWNRN |
| | | | | | LNLGKGGIEIVDITQELQLLFGNVNISYAHGCNLIDDLIIQTNVDFFKKLLRLLSITLSLRHSNGLS |
| | | | | | GVEEKDYILSPIKGKDGVFFDSMKADCTLPKDADANGAYHIALKGLWVLDKINDADDLKKVKM |
| | | | | | AITNKEWLQFVQTKPYRN |
| MG29 | 6542 | MG29-219 effector | Protein | Unknown | MKNQHLFSGFTNQYAISKTLRFELKPVGRTLEHIEKKGLITQDNQRAEDYKEVKILIDEYHKNFI |
| effector | | | | | EKSLDGFALNGLQEYYDLFIKTNLEEIDKKALDKEKDNLRKQIANRFKKIIDKFKSLFAKELIKED |
| | | | | | LIGFVSEENKELVNKFKNFTTYFTGFHENRKNMYVADDKATALAYRLIHENLPKFIGNILIFEKIK |
| | | | | | KDAPDLVSQLNNVLSEMEEIIQGKTLEEIFSLDYFNETLIQTGIDLYNIVLGGRSEDGKDKIKGLN |
| | | | | | EYINLYNQKQTEKKNRQPKLKQLYKQILSDRDSVSFIAEEFKEDTEVLEAIEKFYQGELCNYESD |
| | | | | | GQAINVFNVSKKNLIGNLSSFDLSKVYLRNDRAITYISQQMFSDRSIVGNALQEYYRAQNPQKGK |
| | | | | | EKTENFEKRIDKWVKKSDYFDIQTVQEALSQCSIEAVKEKNKQNAIVNYFADMGLNEEIKMNLF |
| | | | | | DKVIADYKNVKDLLNTSYPENKKLGNQKGKDSDIEKIKTFLDSVMNIIHFIKPLNLKDESKEKDE |
| | | | | | TFYSLFLPLFDQLNKTILLYNQVRNYVTKKPYSTEKIKLNFENSTLLDGWDVNKEQDNTSVILRK |
| | | | | | DGLYYLAIMDKSNKKIFLNAPKADDKKDSFKKMNYKLLPLVNQQLPRVFFAKSRIEYFNPSSKIV |
| | | | | | QNYKNNTHKKGDTFNINDCHALIDFFKASLEKHEDWKHFNFKFSPTKTYQDLSGFYREVEQQG |
| | | | | | YKMTFENIPTDYINEMIEEGKLYLFQIYNKDFSPYSKGKPNMHTIYWKMLFDEQNLKDVVFKLN |
| | | | | | GQAEVFFREKSIKDNIIVHKSNNSIENKNPDNPKKQSKFNYDIIKDKRYTIDKFQFHVPITLNFKAT |
| | | | | | GRDYINEDVNRFLKNNKEVNIIGIDRGERHLAYYMIINQKGEILEQTSFNIISSKHKENKYSTNYH |
| | | | | | ALLEKKEMARDKARKSWDTIGTIKELKEGYISQVVHKIAQLMVKHNAIVVLEYLNPGFKDSRK |
| | | | | | KVEKQVYQKLEKMLIDKLNYLVFKDYEANTTGGVLKALQLANKFTSFDRLGKQSGFLFYVQAA |
| | | | | | LTSKIDPATGFVNFLYPKYESIKKAKKFLDKFDKIIYNNSKNYFEFAFDYNNFTTKAEGTRTKWV |
| | | | | | VCTHGDTRYRYNPQTKTSEEVNITQEIKYLLIKHNIQYENGNCFKNKLIQAEDKKFYSKLLHLLA |
| | | | | | ITVSLRHAKSGTDIDFILSPVADKNGVFFDSRKANDTMPKDADANGAYHIALKGLLALNKISNTP |
| | | | | | DDKLNKVDLKITNKEWLAFAQKKN |
| MG29 | 6543 | MG29-220 effector | Protein | Unknown | MFNHFTHQYSLSKTLRFELKPVGETADYLEDFKSQHLKDFIEKDQQRAEDYKIIKELIDDYHRQY |
| effector | | | | | IEEKLSAPVDPKTGELWLNPEDFEEAYSYYQKLKADQSDPKNRKQWEDIQTELRKKLVKAFTN |
| | | | | | KAGLFKKELITRDLPEFLKRKGQWEENKKVVENFSRFTTYFSGFHENRKNMYTHEDHSTAIAYR |
| | | | | | LMNENLPRFFNNCLAYKHIKDKYNDLPLKINSELQSQLGVTNINDFFQPRYFINLFSQTGIDNYEL |
| | | | | | LIGGKTEENGTKIQGLNEQINLYRQQLQHKMKDEAKADDKKSKRIYDLSGFTHLYKQILSDREK |
| | | | | | ISFIPEAFEDDKQLLASLEEYIATVTDEKEGAIKKLEQSLQKLANADLSKVYIKGAGLTDLSRNMF |
| | | | | | GAYGMITASLSYYADEKLSTKKKPEDYLKQKVYSLDELNHHLIAYIDSKEKDDVLHEQLAKLPN |
| | | | | | PKQPISSYLVSIIKQTQQDVELQTTIDAVKPLLKLESLSKARKAPELDNENDEGAVGFQQVQKIQ |
| | | | | | KMLDGFKSILHAVKLLHLVDGRKPIDMPDKDDGFYNDFAEVYEAYTNLTVTLYNKARNHLTKK |
| | | | | | LYSNDKIKINFDKANLLNGFVESQTDKSYNGTQYGGYLFRKRRDDGGFNYYLGISSKNNLFRFD |
| | | | | | QEISDELSKYERLNYYQAKTNSIYGSSYIGNYGDDKSRLINSVISFLEKNNMEEADNIINGENELVD |
| | | | | | QQKTPTRLFLMILESSQVLLDQVRADKEVNLFERTLMTNLKETLRNFKDKAPALFALRKRDFQS |
| | | | | | IKEFQLAIEEAVTNKSFVYKNISKSEMDEVLINNTMQLFWIYSKDFSEKRIKRRKRIDNLHTLYF |
| | | | | | KALMSGTNDRYDLGKGDVFFRKHSIEKHATHPKGEAIKNKTSNAKNKTSKFNYNLYKDKRYAV |
| | | | | | DKFLFHLSIEINYQKSKSPSSKIFNDQINKTLAQQKETCVIGIDRGERHLLYYTVVNSKGEI |
| MG29 | 6544 | MG29-221 effector | Protein | Unknown | MYFAIRISPWFKALLKLLQIIKYGNYTLLIYFSFIFIGIFIKILTMKNLIDFTGLYSLSKTLRFELIPQ |
| effector | | | | | GKTLENIEKKGLLKQDEARAEKYKKVKKIIDEYHKDFIEKSLNTIKLEGLEYYYDLYLKTNKED |
| | | | | | KEIKEFSKQKERLRKQIANAFKANEKFKTLFLKELIKEDLLSFVSNEEDKADIKEFKDFTTYFIGF |
| | | | | | IIQNRENMYVAEEKATAIAYRLINENLPKFIDNIKIFEKIKNEAPELINQLNQVLSEMEEIVQGKTL |
| | | | | | EEIFSLNYFNQTLTQTGIDLYNIVIGGRTPEENKTKIKGLNEYINTDFNQKQTDKKKRQPKFKQL |
| | | | | | YKQILSDRHSVSFMPESFENDNQLLESIENFYTNELLHYSTEGKSISILEAIKNAVGNLSSFNLSKIY |
| | | | | | LRNDTSLTDISQKVFGDWGVISKALQDYYEKTNPLKPKEKQEKYEERKDKWLKQDIDIQTLQTA |
| | | | | | IDHYENETVKEKNNGNVINDYFAKFGMNNESKIDLLQNVYQNYNIIKDLLNTPFPESEKLGSNKE |
| | | | | | LVNLIKAFLDSIMNVIHFVKPLSLKDSDKEKDESFYSLYTGLYDQLNHTISIYNQVRNYLTQKPYS |
| | | | | | TEKFKLNFENSTLMDGWDLNKEADNTTIILRKDNLFYIGIMDKKNNHIFKHIPEMIDYEPHYEKII |
| | | | | | YKYFPDASKMIPKCSTQLKTVVSHFESNITDKIIEGKSFDSALKITKRIFELNNFVYDDISKTMVLS |
| | | | | | EDNEKRPKMFQKKYLEISKDIDGFKDALKDWINFCIDFLNKYESTKHYSFNFKNSELYNSLDEFY |
| | | | | | GDIDTQTYKITYNNIPVSFIESLVNEGKLYLFQIYNKDFSPFSKGKPNLHTLYWKMLFDEENLNDV |
| | | | | | VYKLNGQAEVFYRKSSIKESNKVIHNANETLTNKNPDNEKATSKFDYDIIKDKRYTLDKFQFHVP |
| | | | | | ITMNFKADGILNINPKVNEFLKNNHDVNIIGIDRGERHLLYYTLINQQGEILEQDTLNIIENEKQK |
| | | | | | VDYHNLLDKKEGTRAEARKDWGTIETIKELKEGYLSQVIHKLTTLMVKHNAIIVMEDLNMGFM |
| | | | | | RGRQKVEKQVYQKFEKMLIDKLNYLVDKSKKANESGGVLRALQLANKFESFKSMGKQNGFIFY |
| | | | | | VPAWNTSKMDPVTGFVNLFDTRYENLEKAKAFFNKFNSICYNQTKDYFEFEFNYANYTAKAEGT |
| | | | | | KTNWILCTYQNRIETFRNTAKNSQWDNREIILTNEFVKLFEQHGIDYKNNNELKSAIVMQTEKAF |
| | | | | | FERLLYLLKLTLQMRNSITGTETDYLISPVANDEGEFYNSRNANNKLPQNADANGAYNIARKGL |
| | | | | | WCLQQINKTDDLKKIKLAISNKEWLQFVQNNN |
| MG29 | 6545 | MG29-222 effector | Protein | Unknown | MEDNNKCIWNEFTNKYKLSKTLRFELKPIGKTLENIKEKGLIEEDKKREKDENKIKKILDNYYK |
| effector | | | | | DFIEISFSKIELNIKDLEEYSEIYNKLKKEKINIELQKKYKKIQDKIRKDTYNKIKTIEPYNKLFKGE |
| | | | | | ILKNILPKWLEKNNYSEDDINLVKSFEKKWKTYFTGFFENRKNVFSEKPIPTSIIYRAINDNLPKFL |
| | | | | | DNLDKFKKIQELDDFSYNSVEEQLSEKLNNTKLNNFFTLDNYNNLLNQKGIDLENLIIGGKSENE |
| | | | | | QKIQGLNELINLYSQKITDKNESRKIKKLKMLPLFKQILSDKESFSDKLEPFKNNKEVIDSINEYYT |
| | | | | | NIYLKNYNKLIELINNINKYDIKQIYIKNDSSLKTISKQILGDWEKIHIGLKEFARKELNINNNTKIN |
| | | | | | SFLKQKYFSVYEIEESIKLLKIDNKQSIYDYLSNFYKKINKPEDNKKNKLITDIEEKYNLFKNIEYN |
| | | | | | NLNRFSNQNIEIIKDFLDSIMDLIHFIKPLYIKNNVKEDDKHQEAYEVDSDFYNQFNDIYKEQLSQI |
| | | | | | IPLYNKTRNFISKKLSSTKKFKLNFDNSNFLKGWSSKFETNSALILRKKNNYYLAIVPKKLNEEEI |
| | | | | | DKLKDFNSLNKIEVLNYDFQKADNKNMARTFIRSKGDNFAPSVEKYNLPVEKILDIYDNGYFRTN |
| | | | | | YREKNIVKFKESLIKLIDYFKEGLKKHESYKHFDFKWKESGEYKDISEFYHDVEVSCYNPSFYEV |
| | | | | | DYNYIENLMEEKKIYLFQIYNKDFSKYSKGKPNLHTIYWRELFSKENFNNIVYKLNGEAEIFFRE |
| | | | | | KAIDKNKTTIHNKNELINNKNPINNKKQSIFEYDLIKDKRYTENKFLFHCPITINFKAKETGTNLH |
| | | | | | KQVN |
| MG29 | 6546 | MG29-223 effector | Protein | Unknown | VEKIRLFLESLLELFHFIKPLKIEEPRKGDKELQGAYEIDSDFYNEFDEIFEKVKGIIPLYNRVRNYI |
| effector | | | | | TQKPFSTKKFKLNFENSTLLNGWDENKIKDNFSVIFRKRNERGQYEYYLGIITPKDKTVENNLEP |
| | | | | | CDSGDCFEMVKYKLLPGPNKMLPKVFFSAKNIEFFNPSEEILSIRNHASHTKNGNPQEGYSKNDF |
| | | | | | NLEDCHKFIDFLKDSLNKHEEWKLYNFRFKETVKYNDVSEFYKDVADQGYKIEFINVDKSYIAK |
| | | | | | LISEGKLYFFKIWNKDFSEYSHGKKNLHTIYWEQLFSDENLKDVVYKLNGEAEIFYRRKSIPLQIT |
| | | | | | IIPKNNPIINKNPINDKETSEFNYDIIKDRRYAVDKFLFIICPITLNFKAEGNGRDMIIRRINMIIIQES |
| | | | | | NEDINILSIDRGERNLAYYTLLNSRGEILVQESFNIISDELRRRFNYLELLSKREGEREEARKQWK |
| | | | | | NMSNIKELKEGYLSHIVHKIAKIAIENNAIIVLEDLNYGFKRERSKIEKQIYQKFEKALIDKFNYL |
| | | | | | MFKERNANETGGALRAYQLTNKFESFKKLGKQSGILFYVPAANTSKICPRTGFVNLLWPKFENI |
| | | | | | KKSQDFFSRFKHIKYIADEDLFEFKFSYSDFNMRSYTGKVKLDEKADSWIVYSNGTRLVNYRNKD |
| | | | | | KNSNWDTKEVYLTKEFKELFEKEGINYEGREDIKAEIANKDSAEFFKRAIELLRYTVQLRNSRIG |
| | | | | | SDEDYILSCVKDKNGEFFDSRNAKEGEPKDADANGAYHIGLKGLMLLERIKKASNPGRINLKIER |
| | | | | | NEFINYVVNKRS |
| MG29 | 6547 | MG29-224 effector | Protein | Unknown | MSKIYQQFTRLYKIQKTLRFGLKPVGETANAIDDFKSQYLQDVVQEDGQRAEDYKVVKDLIDDY |
| effector | | | | | HRVYIEEKLSQPVDRATGEMWVTPEHLEAAYYDYQNLKNNDPKDNKIKKAWAETQKSLRKKL |
| | | | | | VKSFSDNSDLFRKKLITRDLPAFLKVQGKWEENEKAVKSFNKFTTYFKGFHENRKNMYSDEDQS |
| | | | | | TAIAYRVMNENLPKFFNNYLSYQKIKDKLKFSVEKELFTKMGISGIGDVFQPRYFIKLFTQSGLD |
| | | | | | NHQELLGGKTNEDGRKIQGLNEQINLYNQQQSDRQNKLPRFTSLYKQILSDREAHSFIPEVFKDD |
| | | | | | QELLKTLQGYIEKATKKEGLLDNLEKSIALLSTADNEKVYVKTVGLTDISSALFGSYDIIGAALSH |
| | | | | | HAENTAHQNHTKKPASKTLIKKRESFCKQDVFSTAKLDEMITAYIAQLEKTDPLHQQLKKLKTP |
| | | | | | KRPIQIYFLEAFQQAKKEYGFDACIKNITPLLSLESLSKKRQAPTSEGEQGDKGYQQLHSIQKML |
| | | | | | DAFMAISYKLKPLHLVKGRKAIDMPDMDMRFYTKFSESYEDYSDTIINLYNKVRNHLTKKPFSK |
| | | | | | DKIKLNFGNPTLLDGWDANKETDNSSLIFEKDGFYYLGIMHPKHKDLLNYITGIDDIGNDKKTK |
| | | | | | KKELLKKNIEANKNEQHYRKIVYKLLPGANKMLPKVFFSGKRQDYFSPSSEILRIRNSASHSKNG |
| | | | | | NPQEGHAKAEFNIDDCRKIIDFFKVSISKHPEWRAFDFQFSPTQNYQDLSDFYREVEQQAYRVDF |
| | | | | | DLIKQSYIDECIIKGKLFLFQIYNKDFSPYSKGKPNLHTLYWKGLFDPENLKDVVLKLNGEAEVF |
| | | | | | YRPSSINVADRTIHRANEAIDNKNKEFHRKSTSTFAYDIIKDRRYTQDKFQFHVPITLNFKAQGKP |
| | | | | | HFNDTVNLELRNKKDTHVIGIDRGERHLLYYTVVNSKGEHIEQDTLNNISTDQGYAIDYQNKLHK |
| | | | | | REKERDAARKSWSHRKYQRT |
| MG29 | 6548 | MG29-225 effector | Protein | Unknown | MKEDIKTKIDIWDDMTNQYSLSKTLRFELKPIGRTLENIQYIIEEDKQRDKDFQEVKKIMDKYYS |
| effector | | | | | HFISRVLNNKIKISRDSLDEYKEIYFNLKKKIHDNDLKKQLELVQKKITKNISLMIKSDEDFKNIFG |
| | | | | | KEFVKEILKKYLEEKGNQSELELVSKFNDWTTYFTGFYDNRKNVFSDKDIPTSIIYRIVQDNLPKY |
| | | | | | LDNLYNLEQLDKYDIDFSYLNENFGGSIGNKDLKEYFGIENFNEFLSQDGIDKYNYLIGGYSKDM |
| | | | | | KDKVKGINNIIQEYSQQNKEDSDIKKLKFTSLYKQILSDKDEKISFRFFNFNSVDEMLKCIDIFYNK |
| | | | | | LNDENIFENLNFLFDDLNSEIYDKNKVFIKKNKLTFLSNELFGNYSVINYLLKEYVMKNYKDYNT |
| | | | | | EAKFEKWFKGKELFPLSLIEISIARVFDFFDSNSSEIKRFQNIVGVNFINPISFYLTQFKTEGENLFE |
| | | | | | EIKSNYEAYRNLLSSNILDKQNLSQEEKEIKSKQVEILKSFLDSILKLNSFVDIFSTYNVKKDIVEKD |
| | | | | | NDFYNNYEELLLCISEVISIYNQVRNYITKKKTDIKKFKLNFEKSTLLDGWDVNKESANLSVVLRR |
| | | | | | NGKYFLGIMNKKDNKIFDKILPEFITSNNESFEKMEYKLLPGPNKMLPKVFFSEKNIKYFNPSQEI |
| | | | | | IDIRNYSSHSKNGEPQKGYSKKDFNLDDCHKMIDFYKESIEKHDEWKSFKFNFKPTQEYIDLSEF |
| | | | | | YSDVSSQGYKLDFLGISNKYVEKLVEDGKLYLFQIYNKDFSNYSKGKKNLH |
| MG29 | 6549 | MG29-226 effector | Protein | Unknown | MLQNFTNQYQLSKTLRFELRPVGKTKEHIEAKGLIIQDEQRAEEYKEMKKIIDRYHKAFIEDALN |
| effector | | | | | GIAIEGLETYEKLYFAIKDEKGKKEFEKLQDTLRKRIVELFKKIIPKWSTLFKKELIRNELLTFLD |
| | | | | | SEEIPEEQKINEKEIVLKFVDFTTYFTGFHENRANMYIDEALHTAVAYRIVHENLPVFLGNKKTFE |
| | | | | | QIATKYPELIADSKDAIESHLFGAVFEDMFTLAYFSHTLAQNHIDLYNTMIGGKVSNDGSKIQGFN |
| | | | | | EKINLYRQKHGLSKRDLPNLKPLYKQILSDRESLSWLPEAFEDKNELAEAIKTFYQNNIIAFECCD |
| | | | | | GKVNLLEKFPEIFKENQYYDLSKIFIKNDKSLTDIAQAIFGKYGVIKEALWEKHLRDNPKAAKSK |
| | | | | | DISADEERFFNKKDTYFSINDIHIALKEAQSPSDILTYFSNEIKPLTKAVQVAYKTWLDDQEKTERI |
| | | | | | KEVMDALLAWQRFLKPLSVKSDVDRDIAFYATFESYFESLSAVVKLYDKVRNFMTKKPYSLEKF |
| | | | | | KLNFENSTLLDGWDVNKETDNTAILLEKNGLFYLGIMDKKHNRVFKNTPESQDDSSYRKINYKL |
| | | | | | LPGANKMLPKVFFSNSRIDEFAPSSDIISNYKKGTHKKGEIFDLEHCHKLIDFFKSSIQKHEDWKN |
| | | | | | FEFKFSDTASYKDLSGFYREVEQQGYKITYKNISQSYIDTLVSEGKLYLFQIYNKDFSPYSKGTPN |
| | | | | | MHTLYWRALFDEKNLADVVYKLNGQAEVFYRKKSIIYSDEVMQKGHHAKELVGKFDYPIIKDR |
| | | | | | RFAFDKFQFHVPITLNFKAQGYTNLNAIVNEMIASGKEDIKIIGIDRGERHLLYLSLIDAQGKIVE |
| | | | | | QYTLNQIINSYNGKDHVINYHEKLAKKEDERAKARVNWGTVENIKELKEGYMSHVIHRIATLMV |
| | | | | | EHRAIVVLEDLNFGFKRGRFKVEKQVYQKFEKMLIDKLNYLVDKKKSPNELGGVLNALQLINK |
| | | | | | FESFEKMGKQNGFLFYVPAWNTSKIDPVTGFVNLFDTRYASVEKSKEFFGKFKSIRYNSEKDYYE |
| | | | | | FEIDNYTQFNPKAEGTRQNWTICTYGDRVLTYRNSEKLNQWDNKTVQLTDGFKTLLNGQADNL |
| | | | | | KEYIVAQNDKAFFEKLLGLFRLTLQMRNSITGTDIDYLVSPVVDENGVFYDSRVCDDTLPKDADA |
| | | | | | NGAYNIARKGLMVVKKIKEAQDIKKPDLKITNKEWLQFAQR |
| MG29 | 6550 | MG29-227 effector | Protein | Unknown | MGVNTVWNEMTGIYPVSKTIRFELKPIGRTLEYINSKRLLEEDEQRAKDFLEVKKIMNDYFRELI |
| effector | | | | | KTYLANVELDKNLLEQFKEAFIRLKGNKSDSKLKKEYKEFANKLCKELHDNAKLGELEKIKEK |
| | | | | | NFFKPLKEWLKKNNRDEDLKLVISFDGFLIYFRGFFENRKNIFKVPKKDNEDAISTSIVYRTINEN |
| | | | | | LVLFLDDLLKFEELSKLGIDFSEVEENFKEELGGKKLTEFIALVNYSSFLTQEGIDKENLIIGGKTT |
| | | | | | ENGIKLKGINEIINLYSQQKGDKSIRKKKLKQLYKQILSKSNKPSFIPEKLKDDREFIDVIRSADKR |
| | | | | | SDFREIRELFNNLNNYDLNEIYIKRDDLTKLSKDLFSNEEYLNKLIIEYRFAEKTDKQKETEFKKQ |
| | | | | | DYFSIKEIEDAINNEKGLFDLDEDKENFKDKISNNSEHPIVGYFQRFYFKGEDLLKNIEDSYKAVE |
| | | | | | EILERDYSNGTKEFLQQNKEREVEKIKLFLDNSLNILHFIKLLNYKKGKVGSYATLDVDSDFYEG |
| | | | | | YKVDEKEIKGFFWFYNSIKEIVNIYDKARNYVTQKPFSKNKFKLNENNSTLAAGWDKNKEVDNW |
| | | | | | TALFRKDGRYFLGIMDKDSNRLFKEISKNENDDFYEKVEYKLLPSPSKMLPKVFFSDKWKQEHK |
| | | | | | ISEEILKIYENRTFMKGDNFRLEDCHKLVGFYKESIKQHHEWDLYFNFKFKDANKYSDIGEFYRD |
| | | | | | VQNQGYSISFANIDSEVINNYVDKGKLYLFQIYNKDFSENRSEKNKNSKKNLHTLYWNALFSEEN |
| | | | | | LKDVVFKLNGEAELFFRESSIKKEDKVVHKKNEFIKNKDPINGKIESKFSYDLIKNKRYTEDKFFF |
| | | | | | YCPITINFKANGNEKTLNKKVSETIKKNFDGIKVLGIDRGERNLAYFSLINQNGEIEKQDSFNIVLD |
| | | | | | KFNRKNNYLEKLDKKEGQRNEARKNWTKIENIKELKEGYLSQVVHQVVKMAIENNAIIVFEDLN |
| | | | | | SVFKRGRFKIEKQIYQKFEKMLIEKLNYLMFKDIENNKSGGLMKAYQLTPKFYSFKKLGKQTGI |
| | | | | | LFYVNANYTSKIDPKTGFVNLLYPNYENEKQAKEFFKKFDSIKYNKEKDYFEFKFDYKNFKVEE |
| | | | | | KNLPKKSKWVVCSYGERLHQEKDPKSKAWNTVKINLTERLKELFNNSKLDYYEGEDIKEEILKQ |
| | | | | | NNAKFFKELVFLLNKILQIRNSYTSGELRKKIEKRELNFESEGDYILSCVHDKNGEFFDSRAAKEG |
| | | | | | EPKDADANGALNIARKGLIILERIKESREKPDLKIENKKFFDFVNERFN |
| MG29 | 6551 | MG29-228 effector | Protein | Unknown | IKSALEFYYENEIDPEFEEKLKNAKSINKSKKISKEKDSWMKDYVTIATVQKALDVYIESLDTKHD |
| effector | | | | | LRDVYSESLITKYFTNYFKTRLTSGEEVDYIYNIGQKYKAISGILKTKDEDVLKKESVVDQLKDFL |
| | | | | | DSMIDLNRFVQPLNLPSEDLESLSCKDEIFYSDFAPLMEQLNKIIRLYNMARNFITKKKSDEKNIKI |
| | | | | | NFENATLMFGFDINKQYDNSAFILRKNGLYYMGIISKTCKKFIFDDMPRIKDESNAYEKMNYKLI |
| | | | | | NNAYMSLPHVFFSKSRAQEFKPSEEIERIHLTKTFKKGDNFNIKDCHKMIDFFKESIKKNHDWNV |
| | | | | | FNFKFSPTESYNDISEFYREVDIQAYKVWFDEKISDSYINTLVEEGDVYLFKIYGKDFSEHSKGMP |
| | | | | | NLHTMYFKSLFTEENLKDVVYKLCGNGEIFYRKVLIKKEDTITHPANEEIKNKNPLSENKVAIFN |
| | | | | | YEMKKDKRFTVEKYFLNIPIELNFKADGHENINNKINNIIRNNEYANKLSMDRGERNLIHIIVRNQ |
| | | | | | KGETLESIRLNIINNADYKRLLKERENERKTARKGWKTIENIRNIKKGYLSQAVNVVVSLVRKHN |
| | | | | | AVIFMEDLSFGFKNGRKHIETQIYQGFEKALIDKLSYLVFKDKGMDSKEPGGTLNALQLCNKFK |
| | | | | | TFKEMGKQNGIIFFVPAGYTSKICPATGFVNLLKTKYESNKKAKFFFSNFDYIKYNKDKDYFEFS |
| | | | | | FDYNNFIKSNDENSLRNTKGAITKWVACTYGDLRYKYNRNTKASDEINVTQNIKSILDKFNIPYK |
| | | | | | NGKCFKSSLIEKDEKDLFCEILQMLSLTLSLRYSKTGTDIDFILSPVANKNGVFFDSRNATNAMPK |
| | | | | | DGDENGTYHINLKGDWVFEQINKTEDSKKPNLYLRNNEWFAYAQSKCNEIDNAPNWNYRIKKE |
| | | | | | VSKKKTSKKNKKEVVVKK |

mN: 2′-O-methyl modified base N; fN: 2′-Fluoro modified base N; *: phosphorothioate linkage; N: standard ribonucleotide base; AltR1 and AltR2 refer to IDT technologies′ proprietary 5′ and 3′ AltR modifications

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of disrupting a CD38 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said CD38 locus,
wherein said engineered guide RNA is configured to hybridize to a sequence having at least 20-22 consecutive nucleotides having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4466-4503 and 5686; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOS:
4428-4465 and 5685.

2. The method of claim 1, wherein said class 2, type V Cas endonuclease comprises an endonuclease having at least 75% sequence identity to any one of SEQ ID NOs: 1-3470 or a variant thereof.

3. The method of claim 1, wherein said class 2, type V Cas endonuclease comprises an endonuclease having at least 75% sequence identity to any one of SEQ ID NOs: 141, 215, 229, 261, or 1711-1722 or a variant thereof.

4. The method of any one of claims 1-3, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036.

5. The method of any one of claims 1-4, wherein said guide RNA comprises a sequence with at least 80% sequence identity to the non-degenerate nucleotides of SEQ ID NO: 3609.

6. The method of any one of claims 1-5, wherein said engineered guide RNA is configured to hybridize to a sequence having at least 20-22 consecutive nucleotides having at least 80% identity to any one of SEQ ID NOs: 4466, 4467, 4468, 4479, 4484, 4490, 4492, 4493, 4495, 4498.

7. The method of any one of claims 1-6, wherein said engineered guide RNA comprises a nucleotide sequence having at least 80% identity to any one of SEQ ID NOs: 4428, 4429, 4430, 4436, 4441, 4446, 4452, 4454, 4455, 4460, or 4461.

8. The method of any one of claims 1-7, wherein said cell is a eukaryotic cell, T-cell, hematopoietic stem cell, or precursor thereof.

9. A method of disrupting a TIGIT locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said TIGIT locus,
wherein said engineered guide RNA is configured to hybridize to a sequence having at least 20-22 consecutive nucleotides having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4521-4537; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4504-4520.

10. The method of claim 9, wherein said class 2, type V Cas endonuclease comprises an endonuclease having at least 75% sequence identity to any one of SEQ ID NOs: 1-3470 or a variant thereof.

11. The method of claim 9, wherein said class 2, type V Cas endonuclease comprises an endonuclease having at least 75% sequence identity to any one of SEQ ID NOs: 141, 215, 229, 261, or 1711-1722 or a variant thereof.

12. The method of any one of claims 9-11, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036.

13. The method of any one of claims 9-12, wherein said guide RNA comprises a sequence with at least 80% sequence identity to the non-degenerate nucleotides of SEQ ID NO: 3609.

14. The method of any one of claims 9-13, wherein said engineered guide RNA is configured to hybridize to a sequence having at least 20-22 consecutive nucleotides having at least 80% identity to any one of SEQ ID NOs: 4521, 4527, 4528, 4535, or 4536.

15. The method of any one of claims 9-13, wherein said engineered guide RNA comprises a nucleotide sequence having at least 80% identity to any one of SEQ ID NOs: 4504, 4510, 4511, 4518, or 4519.

16. The method of any one of claims 9-15, wherein said cell is a eukaryotic cell, T-cell, hematopoietic stem cell, or precursor thereof.

17. A method of disrupting an AAVS1 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said AAVS1 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4569-4599; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4538-4568.

18. The method of claim 17, wherein said class 2, type V Cas endonuclease comprises an endonuclease having at least 75% sequence identity to any one of SEQ ID NOs: 1-3470 or a variant thereof.

19. The method of claim 18, wherein said class 2, type V Cas endonuclease comprises an endonuclease having at least 75% sequence identity to any one of SEQ ID NOs: 141, 215, 229, 261, or 1711-1722 or a variant thereof.

20. The method of any one of claims 17-19, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, 6033-6036.

21. The method of any one of claims 17-20, wherein said guide RNA comprises a sequence with at least 80% sequence identity to the non-degenerate nucleotides of SEQ ID NO: 3609.

22. The method of any one of claims 17-21, wherein said engineered guide RNA is configured to hybridize to a sequence having at least 20-22 consecutive nucleotides having at least 80% identity to any one of SEQ ID NOs: 4574, 4577, 4578, 4579, 4582, 4584, 4585, 4586, 4587, 4589, 4590, 4591, 4592, 4593, 4595, 4596, or 4598.

23. The method of any one of claims 17-21, wherein said engineered guide RNA comprises a nucleotide sequence having at least 80% identity to any one of SEQ ID NOs: 4543, 4546, 4547, 4548, 4551, 4553, 4554, 4555, 4556, 4558, 4559, 4560, 4561, 4562, 4565, or 4567.

24. The method of any one of claims 17-23, wherein said cell is a eukaryotic cell, T-cell, hematopoietic stem cell, hepatocyte, or precursor thereof.

25. A method of disrupting a B2M locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said B2M locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4676-4751; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4600-4675.

26. The method of claim 25, wherein said class 2, type V Cas endonuclease comprises an endonuclease having at least 75% sequence identity to any one of SEQ ID NOs: 1-3470 or a variant thereof.

27. The method of claim 26, wherein said class 2, type V Cas endonuclease comprises an endonuclease having at least 75% sequence identity to any one of SEQ ID NOs: 141, 215, 229, 261, or 1711-1722 or a variant thereof.

28. The method of any one of claims 25-27, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857 and 6033-6036.

29. The method of any one of claims 25-28, wherein said guide RNA comprises a sequence with at least 80% sequence identity to the non-degenerate nucleotides of SEQ ID NO: 3609.

30. The method of any one of claims 25-29, wherein said engineered guide RNA is configured to hybridize to a sequence having at least 20-22 consecutive nucleotides having at least 80% identity to any one of SEQ ID NOs: 4676, 4678-4687, 4690, 4692, 4698-4707, 4720-4723, 4725-4726, 4732-4733, 4736-4737, 4741, or 4750-4751.

31. The method of any one of claims 25-30, wherein said engineered guide RNA comprises a nucleotide sequence having at least 80% identity to any one of SEQ ID NOS: 4600, 4602-4611, 4614, 4616, 4622-4631, 4644-4647, 4649-4650, 4656-4657, 4660-4661, 4665, or 4674-4675.

32. The method of any one of claims 25-31, wherein said cell is a eukaryotic cell, T-cell, hematopoietic stem cell, or precursor thereof.

33. A method of disrupting a CD2 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said CD2 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4837-4921; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4752-4836.

34. The method of claim 33, wherein said class 2, type V Cas endonuclease comprises an endonuclease having at least 75% sequence identity to any one of SEQ ID NOs: 1-3470 or a variant thereof.

35. The method of claim 34, wherein said class 2, type V Cas endonuclease comprises an endonuclease having at least 75% sequence identity to any one of SEQ ID NOs: 141, 215, 229, 261, or 1711-1722 or a variant thereof.

36. The method of any one of claims 33-35, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036.

37. The method of claim 36, wherein said guide RNA comprises a sequence with at least 80% sequence identity to the non-degenerate nucleotides of SEQ ID NO: 3609.

38. The method of any one of claims 33-37, wherein said engineered guide RNA is configured to hybridize to a sequence having at least 20-22 consecutive nucleotides having at least 80% identity to any one of SEQ ID NOs: 4837, 4844, 4845, 4848, 4857-4858, 4883, 4887, 4892-4893, 4904-4909, 4914, 4916, or 4918.

39. The method of claim 38, wherein said engineered guide RNA has at least 80% sequence identity to any one of SEQ ID NOs: 4752-4836 that target any one of SEQ ID NOs: 4837, 4844, 4845, 4848, 4857-4858, 4883, 4887, 4892-4893, 4904-4909, 4914, 4916, or 4918.

40. The method of any one of claims 33-39, wherein said cell is a eukaryotic cell, T-cell, hematopoietic stem cell, or precursor thereof.

41. A method of disrupting a CD5 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said CD5 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4946-4969; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOS: 4922-4945.

42. The method of claim 41, wherein said class 2, type V Cas endonuclease comprises an endonuclease having at least 75% sequence identity to any one of SEQ ID NOs: 1-3470 or a variant thereof.

43. The method of claim 42, wherein said class 2, type V Cas endonuclease comprises an endonuclease having at least 75% sequence identity to any one of SEQ ID NOs: 141, 215, 229, 261, or 1711-1722 or a variant thereof.

44. The method of any one of claims 41-43, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, and 6033-6036.

45. The method of claim 44, wherein said guide RNA comprises a sequence with at least 80% sequence identity to the non-degenerate nucleotides of SEQ ID NO: 3609.

46. The method of any one of claims 41-45, wherein said engineered guide RNA is configured to hybridize to a sequence having at least 20-22 consecutive nucleotides having at least 80% identity to any one of SEQ ID NOs: 4946-4947, 4949, 4951, 4957-4960, 4963, 4967, or 4969.

47. The method of any one of claims 41-45, wherein said engineered guide RNA has at least 80% sequence identity to any one of SEQ ID NOs: 4922-4945 that target any one of SEQ ID NOs: 4946-4947, 4949, 4951, 4957-4960, 4963, 4967, or 4969.

48. The method of any one of claims 41-47, wherein said cell is a eukaryotic cell, T-cell, hematopoietic stem cell, or precursor thereof.

49. A method of disrupting a mouse TRAC locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said mouse TRAC locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5126-5195, 5682, or 5684; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5056-5125, 5681, or 5683.

50. The method of claim 49, wherein said class 2, type V Cas endonuclease comprises an endonuclease having at least 75% sequence identity to any one of SEQ ID NOs: 1-3470 or a variant thereof.

51. The method of claim 50, wherein said class 2, type V Cas endonuclease comprises an endonuclease having at least 75% sequence identity to any one of SEQ ID NOs: 141, 215, 229, 261, or 1711-1722 or a variant thereof.

52. The method of any one of claims 49-51, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, 3851-3857, or 6033-6036.

53. The method of claim 52, wherein said guide RNA comprises a sequence with at least 80% sequence identity to the non-degenerate nucleotides of SEQ ID NO: 3609.

54. The method of any one of claims 49-53, wherein said engineered guide RNA is configured to hybridize to a sequence having at least 20-22 consecutive nucleotides having at least 80% identity to any one of SEQ ID NOs: 5126-5130, 5133-5143, 5147-5150, 5172-5173, 5184-5189, or 5192-5194.

55. The method of any one of claims 49-53, wherein said engineered guide RNA has at least 80% sequence identity to any one of SEQ ID NOs: 5056-5125 that target any one of SEQ ID NOs: 5126-5130, 5133-5143, 5147-5150, 5172-5173, 5184-5189, or 5192-5194.

56. The method of any one of claims 49-55, wherein said cell is a eukaryotic cell, T-cell, hematopoietic stem cell, or precursor thereof.

57. A method of disrupting a mouse TRBC1 or TRBC2 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said mouse TRBC1 or TRBC2 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5211-5225 or 5247-5267; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5196-5210 or 5226-5246.

58. The method of claim 57, wherein said class 2, type V Cas endonuclease comprises an endonuclease having at least 75% sequence identity to any one of SEQ ID NOs: 1-3470 or a variant thereof.

59. The method of claim 58, wherein said class 2, type V Cas endonuclease comprises an endonuclease having at least 75% sequence identity to any one of SEQ ID NOs: 141, 215, 229, 261, or 1711-1722 or a variant thereof.

60. The method of any one of claims 57-59, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3677-3678, 3695-3696, 3729-3730, 3734-3735, 3851-3857, or 6033-6036.

61. The method of any one of claims 57-60, wherein said guide RNA comprises a sequence with at least 80% sequence identity to the non-degenerate nucleotides of SEQ ID NO: 3609.

62. The method of any one of claims 57-61, wherein said engineered guide RNA is configured to hybridize to a sequence having at least 20-22 consecutive nucleotides having at least 80% identity to any one of SEQ ID NOs: 5211, 5213-5215, 5217, 5221, 5223, 5247, 5249-5250, 5252-5253, 5258-5259, or 5264.

63. The method of any one of claims 57-61, wherein said engineered guide RNA has at least 80% sequence identity to any one of SEQ ID NOs: 5196-5210 or 5226-5246 that target any one of SEQ ID NOs: 5211, 5213-5215, 5217, 5221, 5223, 5247, 5249-5250, 5252-5253, 5258-5259, or 5264.

64. The method of any one of claims 57-63, wherein said cell is a eukaryotic cell, T-cell, hematopoietic stem cell, or precursor thereof.

65. A method of disrupting a human TRBC1 or TRBC2 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said human TRBC1 or TRBC2 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5661-5679; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5642-5660.

66. The method of claim 65, wherein said class 2, type V Cas endonuclease comprises an endonuclease having at least 75% sequence identity to any one of SEQ ID NOs: 1-3470 or a variant thereof.

67. The method of claim 66, wherein said class 2, type V Cas endonuclease comprises an endonuclease having at least 75% sequence identity to any one of SEQ ID NOs: 141, 215, 229, 261, or 1711-1722 or a variant thereof.

68. The method of any one of claims 65-67, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

69. The method of any one of claims 65-68, wherein said guide RNA comprises a sequence with at least 80% sequence identity to the non-degenerate nucleotides of SEQ ID NO: 3609.

70. The method of any one of claims 65-69, wherein said engineered guide RNA is configured to hybridize to a sequence having at least 20-22 consecutive nucleotides having at least 80% identity to any one of SEQ ID NOs: 5661-5663, 5672-5675, or 5678.

71. The method of any one of claims 65-69, wherein said engineered guide RNA has at least 80% sequence identity to any one of SEQ ID NOs: 5642-5660 that target any one of SEQ ID NOs: 5661-5663, 5672-5675, or 5678.

72. The method of any one of claims 65-71, wherein said cell is a eukaryotic cell, T-cell, hematopoietic stem cell, or precursor thereof.

73. A method of disrupting an HPRT locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said HPRT locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5562-5641; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOS: 5482-5561.

74. The method of claim 73, wherein said class 2, type V Cas endonuclease comprises an endonuclease having at least 75% sequence identity to any one of SEQ ID NOs: 1-3470 or a variant thereof.

75. The method of claim 74, wherein said class 2, type V Cas endonuclease comprises an endonuclease having at least 75% sequence identity to any one of SEQ ID NOs: 141, 215, 229, 261, or 1711-1722 or a variant thereof.

76. The method of any one of claims 73-75, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

77. The method of any one of claims 73-76, wherein said guide RNA comprises a sequence with at least 80% sequence identity to the non-degenerate nucleotides of SEQ ID NO: 3609.

78. The method of any one of claims 73-77, wherein said engineered guide RNA is configured to hybridize to a sequence having at least 20-22 consecutive nucleotides having at least 80% identity to any one of SEQ ID NOs: 5562-5564 or 5568.

79. The method of any one of claims 73-77, wherein said engineered guide RNA has at least 80% sequence identity to any one of SEQ ID NOs: 5482-5561 that target any one of SEQ ID NOs: 5562-5564 or 5568.

80. The method of any one of claims 73-80, wherein said cell is a eukaryotic cell, hepatocyte, T-cell, hematopoietic stem cell, or precursor thereof.

81. A method of disrupting an APO-A1 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said APO-A1 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5861-5874; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5847-5860.

82. The method of claim 81, wherein said class 2, type V Cas endonuclease comprises an endonuclease having at least 75% sequence identity to any one of SEQ ID NOs: 1-3470 or a variant thereof.

83. The method of claim 82, wherein said class 2, type V Cas endonuclease comprises an endonuclease having at least 75% sequence identity to any one of SEQ ID NOs: 141, 215, 229, 261, or 1711-1722 or a variant thereof.

84. The method of any one of claims 81-83, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

85. The method of any one of claims 81-84, wherein said guide RNA comprises a sequence with at least 80% sequence identity to the non-degenerate nucleotides of SEQ ID NO: 3609.

86. The method of any one of claims 81-85, wherein said engineered guide RNA is configured to hybridize to a sequence having at least 20-22 consecutive nucleotides having at least 80% identity to any one of SEQ ID NOs: 5861-5866 or 5868-5869.

87. The method of any one of claims 81-85, wherein said engineered guide RNA has at least 80% sequence identity to any one of SEQ ID NOs: 5847-5860 that target any one of SEQ ID NOs: 5861-5866 or 5868-5869.

88. The method of any one of claims 81-87, wherein said cell is a eukaryotic cell, hepatocyte, T-cell, hematopoietic stem cell, or precursor thereof.

89. A method of disrupting an ANGPTL3 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said ANGPTL3 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5953-6030; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5875-5952.

90. The method of claim 89, wherein said class 2, type V Cas endonuclease comprises an endonuclease having at least 75% sequence identity to any one of SEQ ID NOs: 1-3470 or a variant thereof.

91. The method of claim 90, wherein said class 2, type V Cas endonuclease comprises an endonuclease having at least 75% sequence identity to any one of SEQ ID NOs: 141, 215, 229, 261, or 1711-1722 or a variant thereof.

92. The method of any one of claims 89-91, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

93. The method of any one of claims 89-92, wherein said guide RNA comprises a sequence with at least 80% sequence identity to the non-degenerate nucleotides of SEQ ID NO: 3609.

94. The method of any one of claims 89-93, wherein said engineered guide RNA is configured to hybridize to a sequence having at least 20-22 consecutive nucleotides having at least 80% identity to any one of SEQ ID NOs: 5955-5963, 5968-5975, 5979-5987, 5989-5993, 5997, 5999, 6003-6010, 6014-6016, 6024-6025, or 6027-6030.

95. The method of any one of claims 89-93, wherein said engineered guide RNA has at least 80% sequence identity to any one of SEQ ID NOs: 5875-5952 that target any one of SEQ ID NOs: 5955-5963, 5968-5975, 5979-5987, 5989-5993, 5997, 5999, 6003-6010, 6014-6016, 6024-6025, or 6027-6030.

96. The method of any one of claims 89-95, wherein said cell is a eukaryotic cell, hepatocyte, T-cell, hematopoietic stem cell, or precursor thereof.

97. A method of disrupting a human Rosa26 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said human Rosa26 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5013-5055; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 4970-5012.

98. The method of claim 97, wherein said class 2, type V Cas endonuclease comprises an endonuclease having at least 75% sequence identity to any one of SEQ ID NOs: 1-3470 or a variant thereof.

99. The method of claim 98, wherein said class 2, type V Cas endonuclease comprises an endonuclease having at least 75% sequence identity to any one of SEQ ID NOs: 141, 215, 229, 261, or 1711-1722 or a variant thereof.

100. The method of any one of claims 97-99, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

101. The method of any one of claims 97-100, wherein said guide RNA comprises a sequence with at least 80% sequence identity to the non-degenerate nucleotides of SEQ ID NO: 3609.

102. The method of any one of claims 97-101, wherein said cell is a eukaryotic cell, hepatocyte, T-cell, hematopoietic stem cell, or precursor thereof.

103. A method of disrupting a FAS locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said FAS locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5367-5465; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5268-5366.

104. The method of claim 103, wherein said class 2, type V Cas endonuclease comprises an endonuclease having at least 75% sequence identity to any one of SEQ ID NOs: 1-3470 or a variant thereof.

105. The method of claim 104, wherein said class 2, type V Cas endonuclease comprises an endonuclease having at least 75% sequence identity to any one of SEQ ID NOs: 141, 215, 229, 261, or 1711-1722 or a variant thereof.

106. The method of any one of claims 103-105, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

107. The method of any one of claims 103-106, wherein said guide RNA comprises a sequence with at least 80% sequence identity to the non-degenerate nucleotides of SEQ ID NO: 3609.

108. The method of any one of claims 103-107, wherein said cell is a eukaryotic cell, hepatocyte, T-cell, hematopoietic stem cell, or precursor thereof.

109. A method of disrupting a PD-1 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said PD-1 locus,
wherein said engineered guide RNA is configured to hybridize to a sequence having at least 20-22 consecutive nucleotides complementary to a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5474-5481; or
wherein said engineered guide RNA comprises a nucleotide sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 5466-5473.

110. The method of claim 109, wherein said class 2, type V Cas endonuclease comprises an endonuclease having at least 75% sequence identity to any one of SEQ ID NOs: 1-3470 or a variant thereof.

111. The method of claim 110, wherein said class 2, type V Cas endonuclease comprises an endonuclease having at least 75% sequence identity to any one of SEQ ID NOs: 141, 215, 229, 261, or 1711-1722 or a variant thereof.

112. The method of any one of claims 109-111, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

113. The method of any one of claims 109-112, wherein said guide RNA comprises a sequence with at least 80% sequence identity to the non-degenerate nucleotides of SEQ ID NO: 3609.

114. The method of any one of claims 109-113, wherein said cell is a eukaryotic cell, hepatocyte, T-cell, hematopoietic stem cell, or precursor thereof.

115. An engineered nuclease system comprising:
(a) an endonuclease having at least 75% sequence identity to any one of SEQ ID NOs: 215 or a variant thereof; and
(b) an engineered guide RNA, wherein said engineered guide RNA is configured to form a complex with said endonuclease and said engineered guide RNA comprises a spacer sequence configured to hybridize to a target nucleic acid sequence,
wherein said system has reduced immunogenicity when administered to a human subject compared to an equivalent system comprising a Cas9 enzyme.

116. The system of claim 115, wherein said Cas9 enzyme is an SpCas9 enzyme.

117. The system of claim 115-116, wherein said guide RNA comprises a sequence with at least 80% sequence identity to the non-degenerate nucleotides of SEQ ID NO: 3609.

118. The system of any one of claims 115-117, wherein said immunogenicity is antibody immunogenicity.

119. A method of disrupting a mouse HAO-1 locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said mouse HAO-1 locus,
wherein said engineered guide RNA comprises the nucleotides of guide RNAs mH29-1_37, mH29-15_37, mH29-29_37 (SEQ ID NOs: 5779-5781) comprising the nucleotide modifications described in SEQ ID NOs: 5779-5781;
or wherein said engineered guide RNA comprises any one of SEQ ID NOs: 4184-4225.

120. The method of claim 119, wherein said class 2, type V Cas endonuclease comprises an endonuclease having at least 75% sequence identity to any one of SEQ ID NOs: 1-3470 or a variant thereof.

121. The method of claim 120, wherein said class 2, type V Cas endonuclease comprises an endonuclease having at least 75% sequence identity to any one of SEQ ID NOs: 141, 215, 229, 261, or 1711-1722 or a variant thereof.

122. The method of any one of claims 119-121, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

123. The method of any one of claims 119-122, wherein said guide RNA comprises a sequence with at least 80% sequence identity to the non-degenerate nucleotides of SEQ ID NO: 3609.

124. The method of any one of claims 119-123, wherein said cell is a eukaryotic cell, hepatocyte, T-cell, hematopoietic stem cell, or precursor thereof.

125. The method of any one of claims 119-124, wherein said engineered guide RNA comprises the nucleotides of guide RNAs mH29-15_37 or mH29-29_37 (SEQ ID NOs: 5780-5781) comprising the nucleotide modifications described in SEQ ID NOs: 5780-5781.

126. The method of any one of claims 119-125, wherein said method further comprises disrupting expression of glycolate oxidase from said HAO-1 locus.

127. A method of disrupting a human TRAC locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said human TRAC locus,
wherein said engineered guide RNA comprises the nucleotides of MG29-1-TRAC-sgRNA-35 (SEQ ID NOs: 5681 or 5683) comprising the nucleotide modifications described in SEQ ID NOs: 5681 or 5683.

128. The method of claim 127, wherein said class 2, type V Cas endonuclease comprises an endonuclease having at least 75% sequence identity to any one of SEQ ID NOs: 1-3470 or a variant thereof.

129. The method of claim 128, wherein said class 2, type V Cas endonuclease comprises an endonuclease having at least 75% sequence identity to any one of SEQ ID NOs: 141, 215, 229, 261, or 1711-1722 or a variant thereof.

130. The method of any one of claims 127-129, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

131. The method of any one of claims 127-130, wherein said guide RNA comprises a sequence with at least 80% sequence identity to the non-degenerate nucleotides of SEQ ID NO: 3609.

132. The method of any one of claims 127-131, wherein said cell is a eukaryotic cell, hepatocyte, T-cell, hematopoietic stem cell, or precursor thereof.

133. A method of disrupting an albumin locus in a cell, comprising introducing to said cell:
(a) a class 2, type V Cas endonuclease; and
(b) an engineered guide RNA, wherein said engineered guide RNA is configured to form a complex with said endonuclease and said engineered guide RNA comprises a spacer sequence configured to hybridize to a region of said albumin locus,
Wherein said engineered guide RNA comprises the nucleotides of mAlb298-37, mAlb2912-37, mAlb2918-37, or mAlb298-34 (SEQ ID NOs: 5756-5759) comprising the nucleotide modifications described in SEQ ID NOs: 5756-5759; or
wherein said engineered guide RNA comprises the nucleotides of mAlb29-8-44, mAlb29-8-50, mAlb29-8-50b, mAlb29-8-51b, mAlb29-8-52b, mAlb29-8-53b, or mAlb29-8-54b comprising the nucleotide modifications described in Table 5.

134. The method of claim 133, wherein said class 2, type V Cas endonuclease comprises an endonuclease having at least 75% sequence identity to any one of SEQ ID NOs: 1-3470 or a variant thereof.

135. The method of claim 134, wherein said class 2, type V Cas endonuclease comprises an endonuclease having at least 75% sequence identity to any one of SEQ ID NOs: 141, 215, 229, 261, or 1711-1722 or a variant thereof.

136. The method of any one of claims 133-135, wherein said engineered guide RNA comprises a sequence with at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to the non-degenerate nucleotides of any one of SEQ ID NOs: 3471, 3539, 3551-3559, 3608-3609, 3612, 3636-3637, 3640-3641, 3644-3645, 3648-3649, 3652-3653, 3656-3657, 3660-3661, 3664-3667, 3671-3672, 3678, 3695-3696, 3729-3730, 3734-3735, 3851-3857, or 6033-6036.

137. The method of any one of claims 133-136, wherein said guide RNA comprises a sequence with at least 80% sequence identity to the non-degenerate nucleotides of SEQ ID NO: 3609.

138. The method of any one of claims 133-137, wherein said cell is a eukaryotic cell, hepatocyte, T-cell, hematopoietic stem cell, or precursor thereof.

139. The method of any one of claims 133-138, wherein said engineered guide RNA comprises the nucleotides of mAlb298-37, mAlb2912-37, mAlb2918-37, or mAlb298-34 (SEQ ID NOs: 5756-5759) comprising the nucleotide modifications described in SEQ ID NOs: 5756-5759.

140. An engineered guide RNA comprising:
a) a DNA-targeting segment comprising a nucleotide sequence that is complementary to a target sequence in a target DNA molecule; and
b) a protein-binding segment configured to bind to a class 2, type V Cas endonuclease, and
wherein said guide RNA comprises a nucleotide modification pattern depicted in any one of SEQ ID NOs: 5695-5701.

141. The engineered guide RNA of claim 140, wherein said guide RNA comprises mAlb29-8-44, mAlb29-8-50, mAlb29-8-37, or mAlb29-12-44.

142. The engineered guide RNA of claim 140, wherein said guide RNA comprises hH29-4_50, hH29-21_50, hH29-23_50, hH29-41_50, hH29-4_50b, hH29-21_50b, hH29-23_50b, or hH29-41_50b, mH29-1-50, mH29-15-50, mH29-29-50, mH29-1-50b, mH29-15-50b, or mH29-29-50b.

143. The engineered guide RNA of claim 140, wherein said DNA-targeting segment is configured to hybridize to an HAO-1 gene or an albumin gene.

144. The engineered guide RNA of any one of claims 140-142, wherein said class 2, type V Cas endonuclease comprises an endonuclease having at least 75% sequence identity to any one of SEQ ID NOs: 141, 215, 229, 261, or 1711-1722 or a variant thereof.

145. The engineered guide RNA of any one of claims 140-144, wherein said class 2, type V Cas endonuclease comprises an endonuclease having at least 75% sequence identity to SEQ ID NO: 215.

146. An engineered nuclease system comprising:
(a) an endonuclease having at least 75% sequence identity to any one of SEQ ID NOs: 1-3470 or a variant thereof, or a nucleotide sequence encoding said endonuclease; and
(b) a polynucleotide sequence encoding a CRISPR array, wherein said CRISPR array is configured to be processed by said endonuclease to an engineered guide RNA, wherein said engineered guide RNA is configured to form a complex with said endonuclease and said engineered guide RNA comprises a spacer sequence configured to hybridize to a target nucleic acid sequence,
wherein said spacer sequence is configured to hybridize to an albumin gene.

147. The system of claim 146, wherein said polynucleotide sequence comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 5712.

148. The system of claim 146 or 147, wherein said endonuclease comprises an endonuclease having at least 75% sequence identity to any one of SEQ ID NOs: 141, 215, 229, 261, or 1711-1722 or a variant thereof.

149. The system of claim 148, wherein said endonuclease comprises an endonuclease having at least 75% sequence identity to SEQ ID NO: 215.

150. An engineered nuclease system comprising:
(a) an endonuclease having at least 75% sequence identity to SEQ ID NOs: 470 or a variant thereof; and
(b) an engineered guide RNA, wherein said engineered guide RNA is configured to form a complex with said endonuclease and said engineered guide RNA comprises a spacer sequence configured to hybridize to a target nucleic acid sequence.

151. The engineered nuclease system of claim 150, wherein said engineered guide RNA comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 6031.

152. The engineered nuclease system of claim 151 or 152, wherein said endonuclease is configured to be selective for a 5′ PAM sequence comprising SEQ ID NO: 6032.

153. An engineered nuclease system comprising:
(a) an endonuclease having at least at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 2824, 2841, or 2896, or a variant thereof; and
(b) an engineered guide RNA, wherein said engineered guide RNA is configured to form a complex with said endonuclease and said engineered guide RNA comprises a spacer sequence configured to hybridize to a target nucleic acid sequence,
wherein said engineered guide RNA comprises a sequence having at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 6033, 6034, or 6035.

154. The engineered nuclease system of claim 153, wherein said endonuclease has at least 80% sequence identity to SEQ ID NO: 2824 and said engineered guide RNA has at least 80% sequence identity to SEQ ID NO: 6033.

155. The engineered nuclease system of claim 153, wherein said endonuclease has at least 80% sequence identity to SEQ ID NO: 2841 and said engineered guide RNA has at least 80% sequence identity to SEQ ID NO: 6034.

156. The engineered nuclease system of claim 153, wherein said endonuclease has at least 80% sequence identity to SEQ ID NO: 2896 and said engineered guide RNA has at least 80% sequence identity to SEQ ID NO: 6035.

157. The engineered nuclease system of any one of claims 153-156, wherein said endonuclease is configured to be selective for a 5′ PAM sequence comprising any one of SEQ ID NOs: 6037-6039.

158. A lipid nanoparticle comprising:
(a) any of the endonucleases described herein;
(b) any of the engineered guide RNAs described herein:
(c) a cationic lipid;
(d) a sterol;
(e) a neutral lipid; and
(f) a PEG-modified lipid.

159. The lipid nanoparticle of claim 158, wherein said cationic lipid comprises C12-200, said sterol comprises cholesterol, said neutral lipid comprises DOPE, or said PEG-modified lipid comprises DMG-PEG2000.

160. The lipid nanoparticle of claim 158, wherein said cationic lipid comprises 98N12-5 (TETA5-LAP), DLin DMA, DLin-K-DMA (2,2-Dilinoleyl-4-dimethylaminomethyl-[1,3]-dioxolane), DLin-KC2-DMA, DLin-MC3-DMA, or C12-200.

161. An engineered nuclease system comprising:
(a) an endonuclease comprising a sequence having at least 80% sequence identity to any one of SEQ ID NOs: 6274-6281 or 6340-6551, or a variant thereof, wherein said endonuclease is a class 2, type V endonuclease, or a nucleotide sequence encoding said endonuclease; and
(b) an engineered guide RNA, wherein said engineered guide RNA is configured to form a complex with said endonuclease and said engineered guide RNA comprises a spacer sequence configured to hybridize to a target nucleic acid sequence.

162. The engineered nuclease system of claim 161, wherein said endonuclease comprises a sequence having at least 80% sequence identity to any one of SEQ ID NOs: 6274-6281.

163. The engineered nuclease system of claim 161 or 162, wherein said endonuclease is configured to bind to a protospacer adjacent motif (PAM) sequence having at least 80% sequence identity to any one of SEQ ID NOs: 6332-6339.

164. An engineered nuclease system comprising:
(a) an endonuclease comprising a PI (PAM interacting) domain having at least 80% sequence identity to a PI domain of any one of SEQ ID NOs: 2811, 2819, 2878, 2916, 2963, 3009, 6274, 6275, 6276, 6279, 6280, or 6281, or a variant thereof, or a nucleotide sequence encoding said endonuclease, wherein said endonuclease is a class 2, type V endonuclease and said endonuclease is configured to be selective for a 5′ PAM of any one of SEQ ID NOs: 6326-6339; and
(b) an engineered guide RNA, wherein said engineered guide RNA is configured to form a complex with said endonuclease and said engineered guide RNA comprises a spacer sequence configured to hybridize to a target nucleic acid sequence.

165. The engineered nuclease system of claim 164, wherein said endonuclease comprises a sequence having at least 80% sequence identity to any one of SEQ ID NOs: 2811, 2819, 2878, 2916, 2963, 3009, 6274, 6275, 6276, 6279, 6280, or 6281, or a variant thereof.

166. The engineered nuclease system of any one of claims 161-165, wherein said guide RNA comprises a sequence with at least 80% sequence identity to non-degenerate nucleotides of any one of SEQ ID NOs: 6284-6325.

167. The engineered nuclease system of any one of claims 161-166, wherein said guide RNA spacer sequence comprises a sequence complementary to a eukaryotic, fungal, plant, mammalian, or human genomic polynucleotide sequence.

168. The engineered nuclease system of any one of claims 161-167, wherein said endonuclease comprises at least one of a S168R, E172R, N577R, or Y170R mutation when a sequence of said endonuclease is optimally aligned to SEQ ID NO: 215.

169. The engineered nuclease system of any one of claims 161-168, further comprising a single- or double-stranded DNA repair template comprising from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.

170. The engineered nuclease system of claim 169, wherein said first or second homology arm comprises a sequence of at least 40, 80, 120, 150, 200, 300, 500, or 1,000 nucleotides.

171. The engineered nuclease system of claim 169 or 170, wherein said first and second homology arms are homologous to a genomic sequence of a prokaryote, bacteria, fungus, or eukaryote.
